(19) United States
(12) Patent Application Publication
(10) Pub. No.: US 2024/0207263 A1
(43) Pub. Date: Jun. 27, 2024

(54) SELECTIVE MODULATORS OF ATAXIA TELANGIECTASIA MUTATED (ATM) KINASE AND USES THEREOF

(71) Applicant: SUZHOU ZANRONG PHARMA LIMITED, Suzhou, Jiangsu (CN)

(72) Inventors: Ding ZHOU, Shanghai (CN); Ziqiang CHENG, Shanghai (CN); Zheng WANG, Shanghai (CN)

(21) Appl. No.: 18/551,188

(22) PCT Filed: Mar. 15, 2022
(86) PCT No.: PCT/CN2022/080905

§ 371 Date: Sep. 18, 2023

(30) Foreign Application Priority Data
Mar. 17, 2021 (WO) PCT/CN2021/081254

(51) Int. Cl. A61K 31/5025; A61K 31/5377; A61K 45/06; A61N 5/10; A61P 35/00; C07D 487/04; C07D 519/00

(52) U.S. Cl. CPC A61K 31/5025; A61K 31/5377; A61K 45/06; A61N 5/10; A61P 35/00; C07D 487/04; C07D 519/00

(57) ABSTRACT

The specification relates to imidazo [4,5-c] cinnolin-2-one compounds and pharmaceutically acceptable salts thereof, which selectively modulate ataxia telangiectasia mutated (“ATM”) kinase. The specification also relates to pharmaceutical compositions comprising one or more of the compounds and salts thereof as an active ingredient, and to the use of the compounds and salts thereof in the treatment of ATM-associated diseases or conditions, including cancers.

### FIELD OF THE DISCLOSURE

The present application relates to novel substituted imidazo[4,5-c]cinnolin-2-one compounds and pharmaceutically acceptable salts thereof, which selectively modulate ataxia telangiectasia mutated (“ATM”) kinase. The present application also relates to pharmaceutical compositions comprising one or more of the compounds and salts thereof as an active ingredient, and to the use of the compounds and salts thereof in the treatment of ATM-associated diseases or conditions, including cancers.

### BACKGROUND OF THE DISCLOSURE

ATM kinase, a serine/threonine kinase, is named after the autosomal recessive disorder ataxia-telangiectasia (A-T) (Paul, T. T, Annu Rev Biochem 2015, 711-38). ATM plays a central role in the repair of DNA double-strand breaks (DSB), which is very cytotoxic if not timely repaired. DSBs can be repaired by two major pathways: Non-Homologous End Joining (NHEJ) or Homologous Recombination (HR). NHEJ operates throughout the cell cycle, directly resealing the two broken ends with minimal processing. In contrast, HR takes place during the S and G2 phases of the cell cycle and necessitates extensive end processing (or resection). This generates single-stranded DNA that invades the homologous copy of the broken locus which is then used as a template for DNA synthesis (Clouaire, T. et al, DNA Repair (Amst) 2017, 84-91). In comparison, NHEJ is a fast process but error prone; whereas HR is a slower process than NHEJ, but error free. ATM fixes DSBs through HR.

Following DNA DSBs, ATM is recruited by the MRE11-RAD50-NBS1 (MRN) complex which senses and initiates DNA repair. As ATM is brought to the site of DNA damage, it dissociates from inactive homodimers into active monomers and is catalytically activated by autophosphorylation at Ser1981 and other sites, as well as acetylation at Lys3016. ATM then binds to the C terminus of NBS1, a component of the MRN complex, and serves as a transducer and phosphorylates and activates other protein kinases, for example the histone H2A.X (γH2A.X).

ATM is activated by DSBs which can be induced by ionizing radiation, chemotherapy drugs and PARP inhibition. Topoisomerase-I inhibitor (such as irinotecan, topotecan) and PARP inhibitor (such as Olaparib) cause single strand DNA breaks which are converted to DSBs during replication (Choi M. et al, Mol Cancer Ther, 2016, 1781-91). Other anti-cancer treatments such as ionizing radiation (IR), Platinum drugs (Cisplatin), topoisomerase-II inhibitors (doxorubicin, etoposide) directly induces DSBs. Combination of ATM inhibitor with chemotherapy, radiation and PARP inhibitors make cancer cells nearly impossible to repair DSBs which are very cytotoxic. Given the crucial role of ATM played during DSBs, ATM kinase inhibitors are expected to synergize with PARP or Topoisomerase inhibitors or ionizing radiation in the treatment of cancer.

A number of structurally distinct compounds have been reported by showing activity against ATM kinase. WO2015/170081, WO2017/046216 and WO2017/076895 (AstrazenecaAB) reported imidazo[4,5-c]quinolin-2-one compounds as selective modulators of ATM kinase, among which AZD0156 and AZD1390 are potent ATM inhibitors in phase 1 clinical trial:

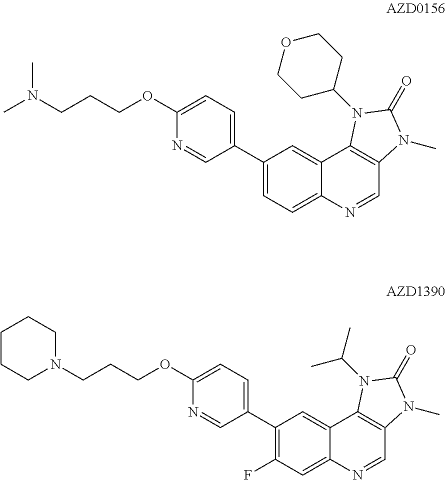

However, both compounds are aldehyde oxidase (AO) substrates with high activity. AO is highly expressed in humans and monkeys, not in dogs and has low expression level in rodents. Compounds metabolized by AO showed high clearance, high PK variability and low oral bioavailability in humans (Garattini, E. et al., Expert Opin Drug Discovery, 2013, 641-54; Zientek, M. et al., Drug Metab Dispos 2010, 1322-7). AO liability can be evaluated in human liver cytosol system. AZD0156's human PK is unexpectedly lower than prediction (Chen et al., AACR, 2018) and a phase 0 clinical PK study was conducted for AZD1390 before making commitment to phase 1 clinical study (NCT03215381 and NCT03423628), further alluding that both AZD0156 and AZD1390 suffer from AO mediated metabolism.

Accordingly, there remains a need to develop new compounds that act against ATM kinase, preferably without AO liability.

### SUMMARY OF THE DISCLOSURE

Disclosed herein are novel substituted imidazo[4,5-c]cinnolin-2-one compounds that possess potent ATM kinase inhibitory activity, do not show AO liability in human liver cytosol and thus have good human pharmacokinetics (PK), low dose and low PK variability. As a result, the compounds of the present application are particularly useful in the treatment of ATM-associated diseases or conditions.

In one aspect, the present disclosure provides compounds of Formula (I):

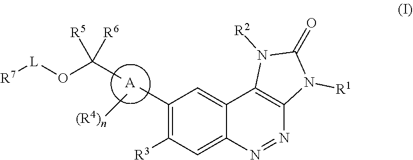

or a pharmaceutically acceptable salt thereof,
wherein:
   Ring A is aryl or heteroaryl;
   R¹ is hydrogen or methyl optionally substituted with 1, 2, or 3 halogens;
   R² is selected from the group consisting of alkyl, alkenyl, alkynyl, cycloalkyl, heterocyclyl, aryl, and heteroaryl, wherein said alkyl, alkenyl, alkynyl, cycloalkyl, heterocyclyl, aryl, and heteroaryl are optionally substituted with one or more R⁸;
   R³ is selected from the group consisting of hydrogen, halogen, hydroxyl, cyano, alkyl, alkenyl, alkynyl, haloalkyl and cycloalkyl;
   each R⁴ is independently selected from hydrogen, halogen, alkyl, alkenyl, alkynyl, and cycloalkyl;
   R⁵ and R⁶ are each independently selected from the group consisting of hydrogen, halogen, cyano, alkyl, alkenyl, alkynyl, haloalkyl and cycloalkyl; or
   R⁵ and R⁶ together with the carbon atom to which they are attached form cycloalkyl, or heterocyclyl;
   L is selected from a direct bond, alkyl or cycloalkyl, wherein said alkyl and cycloalkyl are optionally substituted with one or more R⁹;
   R⁷ is selected from the group consisting of —NR¹⁰R¹¹, —OR¹², —COOH, a saturated or unsaturated heterocyclyl optionally substituted with one or more R¹³
   R⁸ is selected from the group consisting of hydrogen, hydroxyl, halogen, cyano, alkyl, alkenyl, alkynyl, haloalkyl, and alkoxyl;
   R⁹ is selected from the group consisting of hydrogen, halogen, alkyl, haloalkyl, alkenyl, alkynyl, cycloalkyl, and heterocyclyl;
   R¹⁰ and R¹¹ are each independently selected from the group consisting of hydrogen, alkyl, alkenyl, alkynyl, haloalkyl and cycloalkyl; or
   R¹⁰ and R¹¹ together with the nitrogen atom to which they are attached form heterocyclyl optionally containing one or more additional heteroatoms selected from N, O and S and optionally substituted with one or more R¹⁴;
   R¹² and R¹³ are each independently selected from the group consisting of alkyl, alkenyl, alkynyl, haloalkyl, cycloalkyl, and heterocyclyl;
   R¹⁴ is selected from the group consisting of halogen, cyano, sulfonyl, alkyl, alkenyl, alkynyl, haloalkyl, alkylalkoxyl, heterocyclyl, —NR¹⁵R¹⁶, and —OR¹⁷,
   R¹⁵ and R¹⁶ are each independently hydrogen, alkyl, alkenyl, or alkynyl; and
   R¹⁷ is selected from the group consisting of alkyl, alkenyl, alkynyl, haloalkyl, and cycloalkyl, and
   n is 0, 1, 2 or 3.

In another aspect, the present disclosure provides compounds of Formula (II):

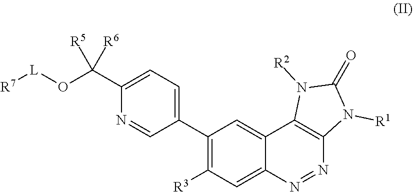

or a pharmaceutically acceptable salt thereof.

In a further aspect, the present disclosure provides compounds selected from the group consisting of:

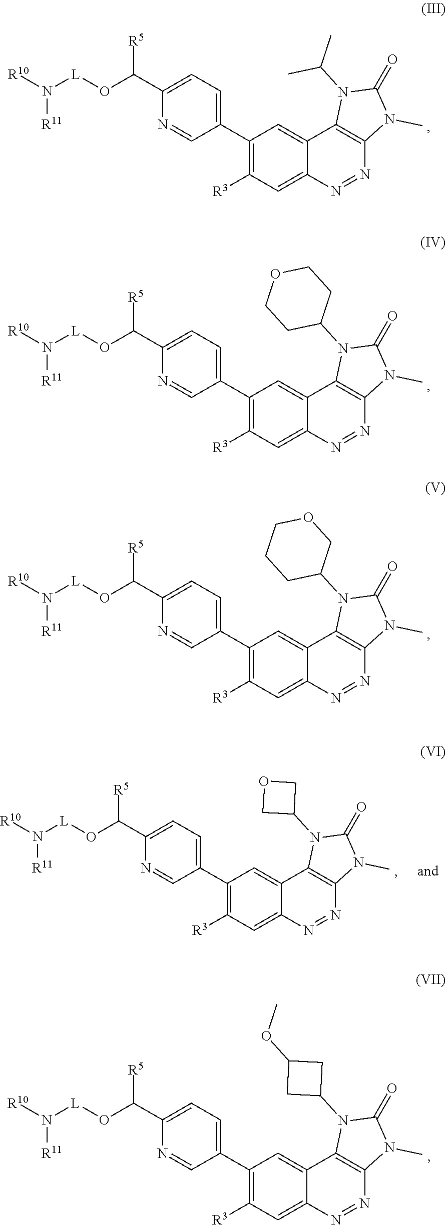

or a pharmaceutically acceptable salt thereof.

In another aspect, there is provided a pharmaceutical composition comprising the compound of Formula (I) or a pharmaceutically acceptable salt thereof, and at least one pharmaceutically acceptable excipient.

In a further aspect, there is provided a method of treating ATM-associated diseases or conditions in a subject in need thereof, comprising administering to the subject a therapeutically effective amount of the compound of Formula (I) or a pharmaceutically acceptable salt thereof.

In a further aspect, there is provided a compound of Formula (I) or a pharmaceutically acceptable salt thereof, for use in the treatment of ATM-associated diseases or conditions.

In a further aspect, there is provided use of a compound of Formula (I) or a pharmaceutically acceptable salt thereof, in the manufacture of a medicament for the treatment of ATM-associated diseases or conditions.

In a further aspect, there is provided a compound of Formula (I) or a pharmaceutically acceptable salt thereof for use in the treatment of ATM-associated diseases or conditions, wherein the compound of Formula (I) is administered simultaneously, separately or sequentially with radiotherapy.

In a further aspect, there is provided a compound of Formula (I) or a pharmaceutically acceptable salt thereof, administered simultaneously, separately or sequentially with at least one additional anti-tumor agent.

In a further aspect, there is provided a compound of Formula (I) or a pharmaceutically acceptable salt thereof, administered simultaneously, separately or sequentially with a PARP inhibitor.

### DETAILED DESCRIPTION OF THE DISCLOSURE

Reference will now be made in detail to certain embodiments of the present disclosure, examples of which are illustrated in the accompanying structures and formulas. While the present disclosure will be described in conjunction with the enumerated embodiments, it will be understood that they are not intended to limit the present disclosure to those embodiments. On the contrary, the present disclosure is intended to cover all alternatives, modifications, and equivalents, which may be included within the scope of the present disclosure as defined by the claims. One skilled in the art will recognize many methods and materials similar or equivalent to those described herein, which could be used in the practice of the present disclosure. The present disclosure is in no way limited to the methods and materials described. In the event that one or more of the incorporated references and similar materials differs from or contradicts this application, including but not limited to defined terms, term usage, described techniques, or the like, the present disclosure controls. All references, patents, patent applications cited in the present disclosure are hereby incorporated by reference in their entireties.

It is appreciated that certain features of the present disclosure, which are, for clarity, described in the context of separate embodiments, can also be provided in combination in a single embodiment. Conversely, various features of the present disclosure, which are, for brevity, described in the context of a single embodiment, can also be provided separately or in any suitable sub-combination. It must be noted that, as used in the specification and the appended claims, the singular forms “a,” “an,” and “the” include plural forms of the same unless the context clearly dictates otherwise. Thus, for example, reference to “a compound” includes a plurality of compounds.

### Definitions

Definitions of specific functional groups and chemical terms are described in more detail below. For purposes of this disclosure, the chemical elements are identified in accordance with the Periodic Table of the Elements, CAS version, Handbook of Chemistry and Physics, 75^{th} Ed., inside cover, and specific functional groups are generally defined as described therein. Additionally, general principles of organic chemistry, as well as specific functional moieties and reactivity, are described in Organic Chemistry, Thomas Sorrell, 2^{nd} Edition, University Science Books, Sausalito, 2006; Smith and March March's Advanced Organic Chemistry, 6^{th} Edition, John Wiley & Sons, Inc., New York, 2007; Larock, Comprehensive Organic Transformations, 3^{rd} Edition, VCH Publishers, Inc., New York, 2018; Carruthers, Some Modern Methods of Organic Synthesis, 4^{th} Edition, Cambridge University Press, Cambridge, 2004; the entire contents of each of which are incorporated herein by reference.

At various places in the present disclosure, linking substituents are described. Where the structure clearly requires a linking group, the Markush variables listed for that group are understood to be linking groups. For example, if the structure requires a linking group and the Markush group definition for that variable lists “alkyl”, then it is understood that the “alkyl” represents a linking alkylene group.

When a bond to a substituent is shown to cross a bond connecting two atoms in a ring, then such substituent may be bonded to any atom in the ring. When a substituent is listed without indicating the atom via which such substituent is bonded to the rest of the compound of a given formula, then such substituent may be bonded via any atom in such formula. Combinations of substituents and/or variables are permissible, but only if such combinations result in stable compounds.

When any variable (e.g., Rⁱ) occurs more than one time in any constituent or formula for a compound, its definition at each occurrence is independent of its definition at every other occurrence. Thus, for example, if a group is shown to be substituted with 0-2 Rⁱ moieties, then the group may optionally be substituted with up to two Rⁱ moieties and Rⁱ at each occurrence is selected independently from the definition of Rⁱ. Also, combinations of substituents and/or variables are permissible, but only if such combinations result in stable compounds.

As used herein, the term “Cᵢ₋ⱼ” indicates a range of the carbon atoms numbers, wherein i and j are integers and the range of the carbon atoms numbers includes the endpoints (i.e. i and j) and each integer point in between, and wherein j is greater than i. For examples, C₁₋₆ indicates a range of one to six carbon atoms, including one carbon atom, two carbon atoms, three carbon atoms, four carbon atoms, five carbon atoms and six carbon atoms. In some embodiments, the term “C₁₋₁₂” indicates 1 to 12, particularly 1 to 10, particularly 1 to 8, particularly 1 to 6, particularly 1 to 5, particularly 1 to 4, particularly 1 to 3 or particularly 1 to 2 carbon atoms.

As used herein, the term “alkyl”, whether as part of another term or used independently, refers to a saturated linear or branched-chain hydrocarbon radical, which may be optionally substituted independently with one or more substituents described below. The term “Cᵢ₋ⱼ alkyl” refers to an alkyl having i to j carbon atoms. In some embodiments, alkyl groups contain 1 to 10 carbon atoms. In some embodiments, alkyl groups contain 1 to 9 carbon atoms. In some embodiments, alkyl groups contain 1 to 8 carbon atoms, 1 to 7 carbon atoms, 1 to 6 carbon atoms, 1 to 5 carbon atoms, 1 to 4 carbon atoms, 1 to 3 carbon atoms, or 1 to 2 carbon atoms. Examples of “C₁₋₁₀ alkyl” include, but are not limited to, methyl, ethyl, propyl, butyl, pentyl, hexyl, heptyl, octyl, nonyl, and decyl. Examples of “C₁₋₆ alkyl” are methyl, ethyl, propyl, isopropyl, n-butyl, i-butyl, s-butyl, t-butyl, n-pentyl, 2-pentyl, 3-pentyl, 2-methyl-2-butyl, 3-methyl-2-butyl, 3-methyl-1-butyl, 2-methyl-1-butyl, 1-hexyl, 2-hexyl, 3-hexyl, 2-methyl-2-pentyl, 3-methyl-2-pentyl, 4-methyl-2-pentyl, 3-methyl-3-pentyl, 2-methyl-3-pentyl, 2,3-dimethyl-2-butyl, 3,3-dimethyl-2-butyl, and the like.

The alkyl groups can be further substituted by substituents which independently replace one or more hydrogen atoms on one or more carbons of the alkyl groups. Examples of such substituents can include, but are not limited to, acyl, alkyl, alkenyl, alkynyl, halogen, hydroxyl, alkoxyl, haloalkyl, haloalkoxyl, alkylcarbonyloxy, arylcarbonyloxy, alkoxycarbonyloxy, aryloxycarbonyloxy, carboxylate, alkylcarbonyl, arylcarbonyl, alkoxycarbonyl, aminocarbonyl, alkylaminocarbonyl, dialkylaminocarbonyl, alkylthiocarbonyl, phosphate, phosphonato, phosphinato, amino (including alkylamino, dialkylamino, arylamino, diarylamino and alkylarylamino), acylamino (including alkylcarbonylamino, arylcarbonylamino, carbamoyl and ureido), amidino, imino, sulfhydryl, alkylthio, arylthio, thiocarboxylate, sulfates, alkylsulfmyl, sulfonate, sulfamoyl, sulfonamido, nitro, trifluoromethyl, cyano, nitro, azido, heterocyclyl, alkylaryl, or an aromatic or heteroaromatic moiety. Alkenyl, alkynyl, cycloalkyl, heterocyclyl, aryl and heteroaryl groups as described below may also be similarly substituted.

As used herein, the term “alkenyl”, whether as part of another term or used independently, refers to linear or branched-chain hydrocarbon radical having at least one carbon-carbon double bond, which may be optionally substituted independently with one or more substituents described herein, and includes radicals having “cis” and “trans” orientations, or alternatively, “E” and “Z” orientations. In some embodiments, alkenyl groups contain 2 to 12 carbon atoms. In some embodiments, alkenyl groups contain 2 to 11 carbon atoms. In some embodiments, alkenyl groups contain 2 to 11 carbon atoms, 2 to 10 carbon atoms, 2 to 9 carbon atoms, 2 to 8 carbon atoms, 2 to 7 carbon atoms, 2 to 6 carbon atoms, 2 to 5 carbon atoms, 2 to 4 carbon atoms, 2 to 3 carbon atoms, and in some embodiments, alkenyl groups contain 2 carbon atoms. Examples of alkenyl group include, but are not limited to, ethylenyl (or vinyl), propenyl, butenyl, pentenyl, 1-methyl-2 buten-1-yl, 5-hexenyl, and the like.

As used herein, the term “alkynyl”, whether as part of another term or used independently, refers to a linear or branched hydrocarbon radical having at least one carbon-carbon triple bond, which may be optionally substituted independently with one or more substituents described herein. In some embodiments, alkenyl groups contain 2 to 12 carbon atoms. In some embodiments, alkynyl groups contain 2 to 11 carbon atoms. In some embodiments, alkynyl groups contain 2 to 11 carbon atoms, 2 to 10 carbon atoms, 2 to 9 carbon atoms, 2 to 8 carbon atoms, 2 to 7 carbon atoms, 2 to 6 carbon atoms, 2 to 5 carbon atoms, 2 to 4 carbon atoms, 2 to 3 carbon atoms, and in some embodiments, alkynyl groups contain 2 carbon atoms. Examples of alkynyl group include, but are not limited to, ethynyl, 1-propynyl, 2-propynyl, and the like.

As used herein, the term “alkoxyl”, whether as part of another term or used independently, refers to an alkyl group, as previously defined, attached to the parent molecule through an oxygen atom. The term “Cᵢ₋ⱼ alkoxy” means that the alkyl moiety of the alkoxy group has i to j carbon atoms. In some embodiments, alkoxy groups contain 1 to 10 carbon atoms. In some embodiments, alkoxy groups contain 1 to 9 carbon atoms. In some embodiments, alkoxy groups contain 1 to 8 carbon atoms, 1 to 7 carbon atoms, 1 to 6 carbon atoms, 1 to 5 carbon atoms, 1 to 4 carbon atoms, 1 to 3 carbon atoms, or 1 to 2 carbon atoms. Examples of “C₁₋₆ alkoxyl” include, but are not limited to, methoxy, ethoxy, propoxy (e.g. n-propoxy and isopropoxy), t-butoxy, neopentoxy, n-hexoxy, and the like.

As used herein, the term “alkylalkoxyl”, whether as part of another term or used independently, refers to an alkyl moiety substituted with one or more alkoxyl moiety. The “alkylalkoxyl” can be bonded to the parent molecular structure through the alkyl group or the alkoxyl group.

As used herein, the term “alkylcycloalkyl”, whether as part of another term or used independently, refers to an alkyl moiety substituted with one or more cycloalkyl moiety. The “alkylcycloalkyl” can be bonded to the parent molecular structure through the alkyl group or the cycloalkyl group.

As used herein, the term “aryl”, whether as part of another term or used independently, refers to monocyclic and polycyclic ring systems having a total of 5 to 20 ring members, wherein at least one ring in the system is aromatic and wherein each ring in the system contains 3 to 12 ring members. Examples of “aryl” include, but are not limited to, phenyl, biphenyl, naphthyl, anthracyl and the like, which may bear one or more substituents. Also included within the scope of the term “aryl”, as it is used herein, is a group in which an aromatic ring is fused to one or more additional rings. In the case of polycyclic ring system, only one of the rings needs to be aromatic (e.g., 2,3-dihydroindole), although all of the rings may be aromatic (e.g., quinoline). The second ring can also be fused or bridged. Examples of polycyclic aryl include, but are not limited to, benzofuranyl, indanyl, phthalimidyl, naphthimidyl, phenanthridinyl, or tetrahydronaphthyl, and the like. Aryl groups can be substituted at one or more ring positions with substituents as described above.

As used herein, the term “cycloalkyl”, whether as part of another term or used independently, refer to a monovalent non-aromatic, saturated or partially unsaturated monocyclic and polycyclic ring system, in which all the ring atoms are carbon and which contains at least three ring forming carbon atoms. In some embodiments, the cycloalkyl may contain 3 to 12 ring forming carbon atoms, 3 to 10 ring forming carbon atoms, 3 to 9 ring forming carbon atoms, 3 to 8 ring forming carbon atoms, 3 to 7 ring forming carbon atoms, 3 to 6 ring forming carbon atoms, 3 to 5 ring forming carbon atoms, 4 to 12 ring forming carbon atoms, 4 to 10 ring forming carbon atoms, 4 to 9 ring forming carbon atoms, 4 to 8 ring forming carbon atoms, 4 to 7 ring forming carbon atoms, 4 to 6 ring forming carbon atoms, 4 to 5 ring forming carbon atoms. Cycloalkyl groups may be saturated or partially unsaturated. Cycloalkyl groups may be substituted. In some embodiments, the cycloalkyl group may be a saturated cyclic alkyl group. In some embodiments, the cycloalkyl group may be a partially unsaturated cyclic alkyl group that contains at least one double bond or triple bond in its ring system.

In some embodiments, the cycloalkyl group may be monocyclic or polycyclic. Examples of monocyclic cycloalkyl group include, but are not limited to, cyclopropyl, cyclobutyl, cyclopentyl, 1-cyclopent-1-enyl, 1-cyclopent-2-enyl, 1-cyclopent-3-enyl, cyclohexyl, 1-cyclohex-1-enyl, 1-cyclohex-2-enyl, 1-cyclohex-3-enyl, cyclohexadienyl, cycloheptyl, cyclooctyl, cyclononyl, cyclodecyl, cycloundecyl and cyclododecyl.

In some embodiments, the cycloalkyl group may be saturated or partially unsaturated polycyclic (e.g., bicyclic and tricyclic) carbocyclic ring system, which can be arranged as a fused, spiro or bridged ring system. As used herein, the term “fused ring” refers to a ring system having two rings sharing two adjacent atoms, the term “spiro ring” refers to a ring systems having two rings connected through one single common atom, and the term “bridged ring” refers to a ring system with two rings sharing three or more atoms. Examples of fused carbocyclyl include, but are not limited to, naphthyl, benzopyrenyl, anthracenyl, acenaphthenyl, fluorenyl and the like. Examples of spiro carbocyclyl include, but are not limited to, spiro[5.5]undecanyl, spiro-pentadienyl, spiro[3.6]-decanyl, and the like. Examples of bridged carbocyclyl include, but are not limited to bicyclo[1,1,1]pentenyl, bicyclo[2,2,1]heptenyl, bicyclo[2.2.1]heptanyl, bicyclo[2.2.2]octanyl, bicyclo[3.3.1]nonanyl, bicyclo[3.3.3]undecanyl, and the like.

As used herein, the term “cyano” refers to —CN.

As used herein, the term “halogen” refers to an atom selected from fluorine (or fluoro), chlorine (or chloro), bromine (or bromo) and iodine (or iodo).

As used herein, the term “haloalkyl” refers to an alkyl group having one or more halogen substituents. Examples of haloalkyl group include, but are not limited to, trifluoromethyl (—CF₃), pentafluoroethyl (—C₂F₅), difluoromethyl (—CHF₂), trichloromethyl (—CCl₃), dichloromethyl (—CHCl₂), pentachloroethyl (—C₂Cl₅), and the like.

As used herein, the term “heteroatom” refers to nitrogen, oxygen, or sulfur, and includes any oxidized form of nitrogen or sulfur, and any quaternized form of a basic nitrogen (including N-oxides).

As used herein, the term “heteroaryl”, whether as part of another term or used independently, refers to an aryl group having, in addition to carbon atoms, one or more heteroatoms. The heteroaryl group can be monocyclic. Examples of monocyclic heteroaryl include, but are not limited to, thienyl, furanyl, pyrrolyl, imidazolyl, pyrazolyl, triazolyl, tetrazolyl, oxazolyl, isoxazolyl, oxadiazolyl, thiazolyl, isothiazolyl, thiadiazolyl, pyridyl, pyridazinyl, pyrimidinyl, pyrazinyl, indolizinyl, purinyl, naphthyridinyl, benzofuranyl and pteridinyl. The heteroaryl group also includes polycyclic groups in which a heteroaromatic ring is fused to one or more aryl, heteroaryl, cycloaliphatic, or heterocyclyl rings, where the radical or point of attachment is on the heteroaromatic ring. Examples of polycyclic heteroaryl include, but are not limited to, indolyl, isoindolyl, benzothienyl, benzofuranyl, benzo[1,3]dioxolyl, dibenzofuranyl, indazolyl, benzimidazolyl, benzthiazolyl, quinolyl, isoquinolyl, dihydroquinolinyl, dihydroisoquinolinyl, tetrahydroquinolinyl, tetrahydroisoquinolinyl, cinnolinyl, phthalazinyl, quinazolinyl, quinoxalinyl, 4H-quinolizinyl, carbazolyl, acridinyl, phenazinyl, phenothiazinyl, phenoxazinyl, tetrahydroquinolinyl, tetrahydroisoquinolinyl, and the like.

As used herein, the term “heterocyclyl” refers to a saturated or partially unsaturated carbocyclyl group in which one or more ring atoms are heteroatoms independently selected from oxygen, sulfur, nitrogen, phosphorus, and the like, the remaining ring atoms being carbon, wherein one or more ring atoms may be optionally substituted independently with one or more substituents. In some embodiments, the heterocyclyl is a saturated heterocyclyl. In some embodiments, the heterocyclyl is a partially unsaturated heterocyclyl having one or more double bonds in its ring system. In some embodiments, the heterocyclyl may contains any oxidized form of carbon, nitrogen or sulfur, and any quaternized form of a basic nitrogen. The heterocyclyl radical may be carbon linked or nitrogen linked where such is possible. In some embodiments, the heterocycle is carbon linked. In some embodiments, the heterocycle is nitrogen linked. For example, a group derived from pyrrole may be pyrrol-1-yl (nitrogen linked) or pyrrol-3-yl (carbon linked). Further, a group derived from imidazole may be imidazol-1-yl (nitrogen linked) or imidazol-3-yl (carbon linked).

Heterocyclyl group may be monocyclic. Examples of monocyclic heterocyclyl include, but are not limited to oxetanyl, 1,1-dioxothietanylpyrrolidyl, tetrahydrofuryl, tetrahydropyranyl, tetrahydrothienyl, azetidinyl, pyrrolyl, furanyl, thienyl, pyrazolyl, imidazolyl, triazolyl, oxazolyl, thiazolyl, piperidyl, piperazinyl, morpholinyl, pyridinyl, pyrazinyl, pyrimidinyl, pyridazinyl, triazinyl, pyridonyl, pyrimidonyl, pyrazinonyl, pyrimidonyl, pyridazonyl, pyrrolidinyl, triazinonyl, and the like.

Heterocyclyl group may be polycyclic, including the fused, spiro and bridged ring systems. The fused heterocyclyl group includes radicals wherein the heterocyclyl radicals are fused with a saturated, partially unsaturated, or fully unsaturated (i.e., aromatic) carbocyclic or heterocyclic ring. Examples of fused heterocyclyl include, but are not limited to, phenyl fused ring or pyridinyl fused ring, such as quinolinyl, isoquinolinyl, quinoxalinyl, quinolizinyl, quinazolinyl, azaindolizinyl, pteridinyl, chromenyl, isochromenyl, indolyl, isoindolyl, indolizinyl, indazolyl, purinyl, benzofuranyl, isobenzofuranyl, benzimidazolyl, benzothienyl, benzothiazolyl, carbazolyl, phenazinyl, phenothiazinyl, phenanthridinyl, imidazo[1,2-a]pyridinyl, [1,2,4]triazolo[4,3-a]pyridinyl, [1,2,3]triazolo[4,3-a]pyridinyl groups, and the like. Examples of spiro heterocyclyl include, but are not limited to, spiropyranyl, spirooxazinyl, 5-aza-spiro[2.4]heptanyl, 6-aza-spiro[2.5]octanyl, 6-aza-spiro[3.4]octanyl, 2-oxa-6-aza-spiro[3.3]heptanyl, 2-oxa-6-aza-spiro[3.4]octanyl, 6-aza-spiro[3.5]nonanyl, 7-aza-spiro[3.5]nonanyl, 1-oxa-7-aza-spiro[3.5]nonanyl and the like. Examples of bridged heterocyclyl include, but are not limited to, 3-aza-bicyclo[3.1.0]hexanyl, 8-aza-bicyclo[3.2.1]octanyl, 1-aza-bicyclo[2.2.2]octanyl, 2-aza-bicyclo[2.2.1]heptanyl, 1,4-diazabicyclo[2.2.2]octanyl, and the like.

As used herein, the term “hydroxyl” refers to —OH.

As used herein, the term “sulfonyl” refers to —SO₂R′, wherein R′ is selected from hydrogen, alkyl, alkenyl, alkynyl, haloalkyl, cycloalkyl, heterocyclyl, aryl, or heteroaryl.

As used herein, the term “partially unsaturated” refers to a radical that includes at least one double or triple bond. The term “partially unsaturated” is intended to encompass rings having multiple sites of unsaturation, but is not intended to include aromatic (i.e., fully unsaturated) moieties.

As used herein, the term “substituted”, whether preceded by the term “optionally” or not, means that one or more hydrogens of the designated moiety are replaced with a suitable substituent. It will be understood that “substitution” or “substituted with” includes the implicit proviso that such substitution is in accordance with permitted valence of the substituted atom and that the substitution results in a stable or chemically feasible compound, e.g., which does not spontaneously undergo transformation such as by rearrangement, cyclization, elimination, etc. Unless otherwise indicated, an “optionally substituted” group may have a suitable substituent at each substitutable position of the group, and when more than one position in any given structure may be substituted with more than one substituent selected from a specified group, the substituent may be either the same or different at every position. It will be understood by those skilled in the art that substituents can themselves be substituted, if appropriate. Unless specifically stated as “unsubstituted”, references to chemical moieties herein are understood to include substituted variants. For example, reference to an “aryl” group or moiety implicitly includes both substituted and unsubstituted variants.

### Compound

The present disclosure provides novel substituted imidazo[4,5-c]cinnolin-2-one compounds or pharmaceutically acceptable salts thereof, synthetic methods for making the compounds, pharmaceutical compositions containing them and various uses of the disclosed compounds.

In one aspect, the present disclosure provides a compound of Formula (I):

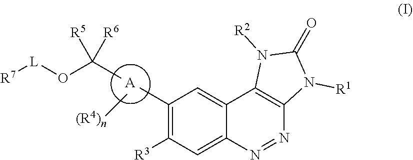

or a pharmaceutically acceptable salt thereof,
wherein:
   Ring A is aryl or heteroaryl;
   R¹ is hydrogen or methyl optionally substituted with 1, 2, or 3 halogens;
   R² is selected from the group consisting of alkyl, alkenyl, alkynyl, cycloalkyl, heterocyclyl, aryl, and heteroaryl, wherein said alkyl, alkenyl, alkynyl, cycloalkyl, heterocyclyl, aryl, and heteroaryl are optionally substituted with one or more R⁸;
   R³ is selected from the group consisting of hydrogen, halogen, hydroxyl, cyano, alkyl, alkenyl, alkynyl, haloalkyl and cycloalkyl;
   each R⁴ is independently selected from hydrogen, halogen, alkyl, alkenyl, alkynyl, and cycloalkyl;
   R⁵ and R⁶ are each independently selected from the group consisting of hydrogen, halogen, cyano, alkyl, alkenyl, alkynyl, haloalkyl and cycloalkyl; or
   R⁵ and R⁶ together with the carbon atom to which they are attached form cycloalkyl, or heterocyclyl;
   L is selected from a direct bond, alkyl, cycloalkyl or cycloalkylalkyl, wherein said alkyl, cycloalkyl and cycloalkylalkyl are optionally substituted with one or more R⁹;
   R⁷ is selected from the group consisting of —NR¹⁰R¹¹, —OR¹², —COOH, a saturated or unsaturated heterocyclyl optionally substituted with one or more R¹³;
   R⁸ is selected from the group consisting of hydrogen, hydroxyl, halogen, cyano, alkyl, alkenyl, alkynyl, haloalkyl, and alkoxyl;
   R⁹ is selected from the group consisting of hydrogen, halogen, alkyl, haloalkyl, alkenyl, alkynyl, cycloalkyl, and heterocyclyl;
   R¹⁰ and R¹¹ are each independently selected from the group consisting of hydrogen, alkyl, alkenyl, alkynyl, haloalkyl and cycloalkyl; or
   R¹⁰ and R¹¹ together with the nitrogen atom to which they are attached form heterocyclyl optionally containing one or more additional heteroatoms selected from N, O and S and optionally substituted with one or more R¹⁴;
   R¹² and R¹³ are each independently selected from the group consisting of alkyl, alkenyl, alkynyl, haloalkyl, cycloalkyl, and heterocyclyl;
   R¹⁴ is selected from the group consisting of halogen, cyano, sulfonyl, alkyl, alkenyl, alkynyl, haloalkyl, alkylalkoxyl, heterocyclyl, —NR¹⁵R¹⁶, and —OR¹⁷,
   R¹⁵ and R¹⁶ are each independently hydrogen, alkyl, alkenyl, or alkynyl; and
   R¹⁷ is selected from the group consisting of alkyl, alkenyl, alkynyl, haloalkyl, and cycloalkyl, and
   n is 0, 1, 2 or 3.

In some embodiments, Ring A is aryl.

In some embodiments, Ring A is heteroaryl. In certain embodiments, Ring A is 5- to 12-membered heteroaryl. In certain embodiments, Ring A is 5- to 10-membered heteroaryl. In certain embodiments, Ring A is 5- to 8-membered heteroaryl. In certain embodiments, Ring A is 5- to 6-membered heteroaryl.

In certain embodiments, Ring A is selected from the group consisting of thienyl, furanyl, pyrrolyl, imidazolyl, pyrazolyl, oxazolyl, isoxazolyl, thiazolyl, isothiazolyl, pyridyl, pyridazinyl, pyrimidinyl and pyrazinyl.

In certain embodiments, Ring A is selected from pyridyl, pyridazinyl, pyrimidinyl or pyrazinyl. In certain embodiments, Ring A is pyridyl.

In some embodiments, R¹ is hydrogen.

In some embodiments, R¹ is methyl optionally substituted with 1, 2, or 3 halogens. In certain embodiments, R¹ is methyl optionally substituted with 1, 2, or 3 fluoro. R¹ is methyl or trifluoromethyl.

In some embodiments, R² is alkyl optionally substituted with one or more R⁸, and R⁸ is selected from the group consisting of hydrogen, hydroxyl, halogen, cyano, and alkoxyl. In certain embodiments, R² is C₁₋₆ alkyl, C₁₋₅ alkyl, C₁₋₄ alkyl, or C₁₋₃ alkyl, which is optionally substituted with one or more R⁸, and R⁸ is selected from the group consisting of hydrogen, hydroxyl, halogen, cyano, and alkoxyl.

In certain embodiments, R² is n-propyl or iso-propyl.

In some embodiments, R² is cycloalkyl optionally substituted with one or more R⁸, and R⁸ is selected from the group consisting of hydrogen, hydroxyl, halogen, cyano, alkyl, haloalkyl, and alkoxyl. In certain embodiments, R² is C₃₋₁₂ cycloalkyl, C₃₋₁₀ cycloalkyl, C₃₋₈ cycloalkyl, C₃₋₆ cycloalkyl, or C₃₋₅ cycloalkyl, which is optionally substituted with one or more R⁸, and R⁸ is selected from the group consisting of hydrogen, hydroxyl, halogen, cyano, alkyl, haloalkyl, and alkoxyl.

In certain embodiments, R² is selected from the group consisting of cyclopropyl, cyclobutyl, cyclopentyl and cyclohexyl, each of which is optionally substituted with one or more R⁸, and R⁸ is selected from hydroxyl, methyl, trifluoromethyl or methoxy.

In certain embodiments, R² is

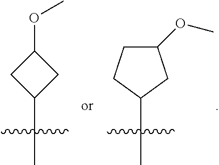

In some embodiments, R² is heterocyclyl optionally substituted with one or more R⁸, and R⁸ is selected from the group consisting of hydrogen, hydroxyl, halogen, cyano, alkyl, haloalkyl and alkoxyl. In certain embodiments, R² is C₃₋₁₂ heterocyclyl, C₃₋₁₀ heterocyclyl, C₃₋₈ heterocyclyl, C₃₋₆ heterocyclyl, or C₃₋₅ heterocyclyl, which is optionally substituted with one or more R⁸, and R⁸ is selected from the group consisting of hydrogen, hydroxyl, halogen, cyano, alkyl, haloalkyl, and alkoxyl.

In certain embodiments, R² is selected from the group consisting of oxetanyl, tetrahydrofuranyl and tertrahydropyranyl, each of which is optionally substituted with one or more R⁸, and R⁸ is selected from the group consisting of hydroxyl, methyl, trifluoromethyl and methoxy.

In certain embodiments, R² is

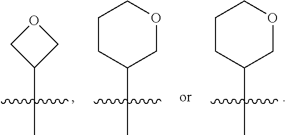

In some embodiments, R² is heteroaryl optionally substituted with one or more R⁸, and R⁸ is selected from the group consisting of hydrogen, hydroxyl, halogen, cyano, alkyl, haloalkyl and alkoxyl. In certain embodiments, R² is 5- to 12-membered heteroaryl, 5- to 10-membered heteroaryl, 5- to 8-membered heteroaryl, 5- to 7-membered heteroaryl, or 5- to 6-membered heteroaryl, which is optionally substituted with one or more R⁸, and R⁸ is selected from the group consisting of hydrogen, hydroxyl, halogen, cyano, alkyl, haloalkyl and alkoxyl.

In certain embodiments, R² is pyridine or pyrazole, each of which is optionally substituted with one or more R⁸, and R⁸ is selected from the group consisting of hydroxyl, methyl, trifluoromethyl and methoxy.

In some embodiments, R³ is hydrogen.

In some embodiments, R³ is halogen. In certain embodiments, R³ is fluoro, choro or bromo. In certain embodiments, R³ is fluoro.

In some embodiments, R⁴ is hydrogen.

In some embodiments, R⁴ is alkyl. In certain embodiments, R⁴ is C₁₋₆ alkyl. In certain embodiments, R⁴ is C₁₋₅ alkyl, C₁₋₄ alkyl, C₁₋₃ alkyl or C₁₋₂ alkyl.

In some embodiments, n is 0. In some embodiments, n is 1. In some embodiments, n is 2. In some embodiments, n is 3.

In some embodiments, one of R⁵ and R⁶ is hydrogen, and the other is alkyl. In certain embodiments, one of R⁵ and R⁶ is hydrogen, and the other is C₁₋₆ alkyl, C₁₋₅ alkyl, C₁₋₄ alkyl, or C₁₋₃ alkyl. In certain embodiments, one of R⁵ and R⁶ is hydrogen, and the other is selected from methyl, ethyl, n-propyl, i-propyl, or 3-methyl-1-butyl.

In some embodiments, one of R⁵ and R⁶ is hydrogen, and the other is haloalkyl. In certain embodiments, one of R⁵ and R⁶ is hydrogen, and the other is C₁₋₆ haloalkyl, C₁₋₅ haloalkyl, C₁₋₄ haloalkyl, or C₁₋₃ haloalkyl. In certain embodiments, one of R⁵ and R⁶ is hydrogen, and the other is methyl optionally substituted with 1, 2, or 3 halogens. In certain embodiments, one of R⁵ and R⁶ is hydrogen, and the other is methyl optionally substituted with 1, 2, or 3 fluoro atoms. In certain embodiments, one of R⁵ and R⁶ is hydrogen, and the other is trifluoromethyl.

In some embodiments, both of R⁵ and R⁶ are hydrogen.

In some embodiments, both of R⁵ and R⁶ are alkyl. In certain embodiments, R⁵ and R⁶ are independently C₁₋₆ alkyl, C₁₋₅ alkyl, C₁₋₄ alkyl, or C₁₋₃ alkyl. In certain embodiments, both R⁵ and R⁶ are methyl.

In some embodiments, R⁵ and R⁶ together with the carbon atom to which they are attached form cycloalkyl optionally substituted with one or more R⁹. In certain embodiments, R⁵ and R⁶ together with the carbon atom to which they are attached form C₃₋₁₂ cycloalkyl, C₃₋₁₀ cycloalkyl, C₃₋₈ cycloalkyl, C₃₋₆ cycloalkyl, or C₃₋₅ cycloalkyl, each of which is optionally substituted with one or more R⁹. In certain embodiments, R⁵ and R⁶ together with the carbon atom to which they are attached form cyclopropyl, cyclobutyl, cyclopentyl or cyclohexyl.

In some embodiments, R⁵ and R⁶ together with the carbon atom to which they are attached form heterocyclyl optionally substituted with one or more R⁹. In certain embodiments, R⁵ and R⁶ together with the carbon atom to which they are attached form 3- to 12-membered heterocyclyl, 3- to 10-membered heterocyclyl, 3- to 8-membered heterocyclyl, 3- to 6-membered heterocyclyl, or 3- to 5-membered heterocyclyl, each of which is optionally substituted with one or more R⁹.

In certain embodiments, R⁵ and R⁶ together with the carbon atom to which they are attached form a group selected from the group consisting of:

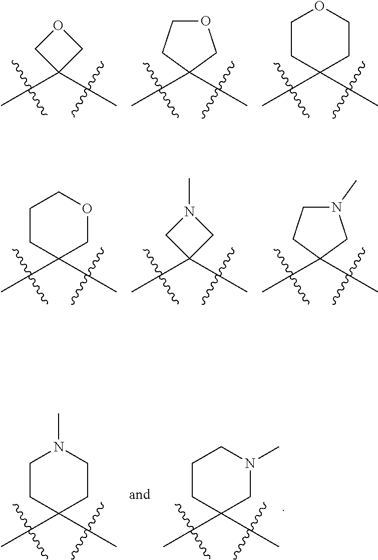

In some embodiments, L is a direct bond.

In some embodiments, L is alkyl optionally substituted with one or more R⁹. In certain embodiments, L is C₁₋₆ alkyl, C₁₋₅ alkyl, C₁₋₄ alkyl, or C₁₋₃ alkyl, each of which is optionally substituted with one or more R⁹. In certain embodiments, L is methyl, ethyl, propyl or butyl optionally substituted with one or more R⁹. In some embodiments, each R⁹ is independently selected from the group consisting of hydrogen, halogen, and cycloalkyl. In certain embodiments, R⁹ is halogen. In certain embodiments, R⁹ is fluoro. In certain embodiments, R⁹ is selected from C₃₋₁₂ cycloalkyl, C₃₋₁₀ cycloalkyl, C₃₋₈ cycloalkyl, C₃₋₆ cycloalkyl, or C₃₋₅ cycloalkyl. In certain embodiments, R⁹ is cyclopropyl.

In certain embodiments, L is selected from methyl, ethyl, n-propyl, isopropyl, or isobutyl.

In some embodiments, L is cycloalkyl optionally substituted with one or more R⁹. In certain embodiments, L is C₃₋₁₂ cycloalkyl, C₃₋₁₀ cycloalkyl, C₃₋₈ cycloalkyl, C₃₋₆ cycloalkyl, or C₃₋₅ cycloalkyl, each of which is optionally substituted with one or more R⁹. In some embodiments, each R⁹ is selected from the group consisting of hydrogen, halogen, alkyl, haloalkyl and cycloalkyl.

In certain embodiments, L is cyclopropyl or cyclobutyl.

In some embodiments, L is cycloalkylalkyl optionally substituted with one or more R⁹. In certain embodiments, L is selected from (C₃₋₆ cycloalkyl)(C₁₋₆ alkyl), (C₃₋₅ cycloalkyl)(C₁₋₅ alkyl), or (C₃₋₄ cycloalkyl)(C₁₋₄ alkyl), each of which is optionally substituted with one or more R⁹. In some embodiments, each R⁹ is selected from the group consisting of hydrogen, halogen, alkyl, haloalkyl and cycloalkyl.

In certain embodiments, L is cyclopropylmethyl. In certain embodiments, L is

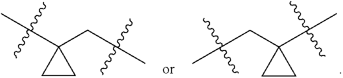 [SMILES: *CC1(*)CC1 ; *CC1(*)CC1]

In some embodiments, R⁷ is —NR¹⁰R¹¹.

In certain embodiments, R¹⁰ and R¹¹ are each independently alkyl. In certain embodiments, R¹⁰ and R¹¹ are each independently C₁₋₆ alkyl, C₁₋₅ alkyl, C₁₋₄ alkyl, or C₁₋₃ alkyl. In certain embodiments, R¹⁰ and R¹¹ are both C₁₋₃ alkyl. In certain embodiments, R¹⁰ and R¹¹ are both methyl.

In certain embodiments, one of R¹⁰ and R¹¹ is hydrogen, and the other is alkyl. In certain embodiments, one of R¹⁰ and R¹¹ is hydrogen, and the other is C₁₋₆ alkyl, C₁₋₅ alkyl, C₁₋₄ alkyl, or C₁₋₃ alkyl. In certain embodiments, one of R¹⁰ and R¹¹ is hydrogen, and the other is methyl.

In some embodiments, R¹⁰ and R¹¹ together with the nitrogen atom to which they are attached form a saturated heterocyclyl optionally containing one or more additional heteroatoms selected from N, O and S and optionally substituted with one or more R¹⁴.

In certain embodiments, R¹⁰ and R¹¹ together with the nitrogen atom to which they are attached form a saturated heterocyclyl selected from the group consisting of:

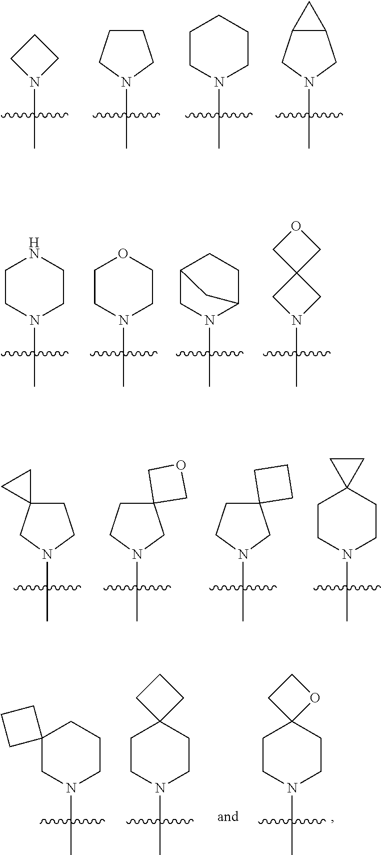

each of which is optionally substituted with one or more R¹⁴.

In certain embodiments, each R¹⁴ is independently selected from the group consisting of halogen, cyano, sulfonyl, alkyl, haloalkyl, alkylalkoxyl, —NR¹⁵R¹⁶, and OR¹⁷.

In certain embodiments, R¹⁴ is halogen. In certain embodiments, R¹⁴ is fluoro.

In certain embodiments, R¹⁴ is cyano.

In certain embodiments, R¹⁴ is sulfonyl. In certain embodiments, R¹⁴ is —SO₂(CH₃).

In certain embodiments, R¹⁴ is alkyl. In certain embodiments, R¹⁴ is C₁₋₆ alkyl, C₁₋₅ alkyl, C₁₋₄ alkyl, or C₁₋₃ alkyl. In certain embodiments, R¹⁴ is methyl.

In certain embodiments, R¹⁴ is haloalkyl. In certain embodiments, R¹⁴ is methyl substituted with 1, 2 or 3 halogens. In certain embodiments, R¹⁴ is trifluoromethyl.

In certain embodiments, R¹⁴ is alkylalkoxyl. In certain embodiments, R¹⁴ is selected from (C₁₋₆ alkyl)(C₁₋₆ alkoxyl), (C₁₋₅ alkyl)(C₁₋₅ alkoxyl), (C₁₋₄ alkyl)(C₁₋₄ alkoxyl), or (C₁₋₃ alkyl)(C₁₋₃ alkoxyl). In certain embodiments, R¹⁴ is methylmethoxyl.

In certain embodiments, R¹⁴ is —NR¹⁵R¹⁶.

In certain embodiments, R¹⁵ and R′⁶ are each independently alkyl. In certain embodiments, R¹⁵ and R¹⁶ are each independently C₁₋₆ alkyl, C₁₋₅ alkyl, C₁₋₄ alkyl, or C₁₋₃ alkyl. In certain embodiments, R¹⁵ and R¹⁶ are both C₁₋₃ alkyl. In certain embodiments, R¹⁵ and R¹⁶ are both methyl.

In certain embodiments, one of R¹⁵ and R¹⁶ is hydrogen, and the other is alkyl. In certain embodiments, one of R¹⁵ and R¹⁶ is hydrogen, and the other is C₁₋₆ alkyl, C₁₋₅ alkyl, C₁₋₄ alkyl, or C₁₋₃ alkyl. In certain embodiments, one of R¹⁵ and R¹⁶ is hydrogen, and the other is methyl.

In some embodiments, R⁷ is —OR¹⁷.

In certain embodiments, R¹⁷ is selected from alkyl or haloalkyl.

In certain embodiments, R¹⁷ is alkyl, such as C₁₋₆ alkyl, C₁₋₅ alkyl, C₁₋₄ alkyl, or C₁₋₃ alkyl. In certain embodiments, R¹⁷ is methyl, ethyl, or isopropyl.

In certain embodiments, R¹⁷ is haloalkyl. In certain embodiments, R¹⁷ is methyl substituted with 1, 2 or 3 halogens. In certain embodiments, R¹⁷ is monofluoromethyl, difluoromethyl or trifluoromethyl.

In some embodiments, R⁷ is —COOH.

In some embodiments, R⁷ is heterocyclyl optionally substituted with R¹³.

In certain embodiments, R⁷ is selected from the group consisting of:

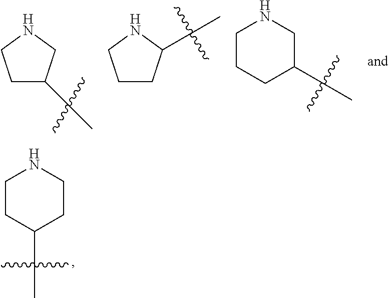

each of which is optionally substituted with one or more R¹³.

In certain embodiments, R¹³ is selected from the group consisting of alkyl, haloalkyl, cycloalkyl, and heterocyclyl. In certain embodiments, R¹³ is alkyl, such as C₁₋₆ alkyl, C₁₋₅ alkyl, C₁₋₄ alkyl, or C₁₋₃ alkyl. In certain embodiments, R¹³ is methyl.

In a further aspect, the present disclosure provides compounds of Formula (II):

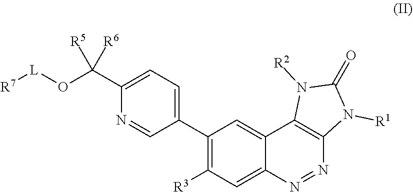

wherein R¹, R², R³, R⁵, R⁶, R⁷ and L are as defined as supra.

In some embodiments, in Formula (II), one of R⁵ and R⁶ is hydrogen, and the other is alkyl or haloalkyl.

In certain embodiments, one of R⁵ and R⁶ is hydrogen, and the other is alkyl. In certain embodiments, one of R⁵ and R⁶ is hydrogen, and the other is C₁₋₆ alkyl, C₁₋₅ alkyl, C₁₋₄ alkyl, or C₁₋₃ alkyl. In certain embodiments, one of R⁵ and R⁶ is hydrogen, and the other is selected from methyl, ethyl, n-propyl, i-propyl, or 3-methyl-1-butyl.

In certain embodiments, one of R⁵ and R⁶ is hydrogen, and the other is haloalkyl. In certain embodiments, one of R⁵ and R⁶ is hydrogen, and the other is C₁₋₆ haloalkyl, C₁₋₅ haloalkyl, C₁₋₄ haloalkyl, or C₁₋₃ haloalkyl. In certain embodiments, one of R⁵ and R⁶ is hydrogen, and the other is methyl optionally substituted with 1, 2, or 3 halogens. In certain embodiments, one of R⁵ and R⁶ is hydrogen, and the other is methyl optionally substituted with 1, 2, or 3 fluoro atoms. In certain embodiments, one of R⁵ and R⁶ is hydrogen, and the other is trifluoromethyl.

In another aspect, the present disclosure provides compounds selected from the group consisting of:

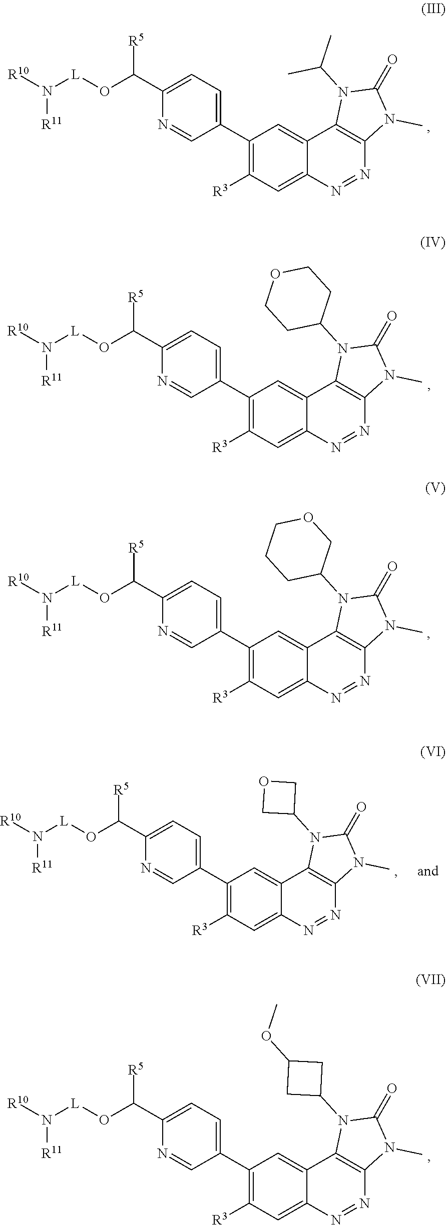

wherein R³, R⁵, L, R¹¹ and R¹² are as defined as supra.

In certain embodiments, in any of Formula (III) to (VII),
R³ is hydrogen or fluoro;
R⁵ is hydrogen, alkyl or haloalkyl;
L is alkyl optionally substituted with one or more R⁹;
R⁹ is selected from the group consisting of hydrogen, halogen, and cycloalkyl;
R¹⁰ and R¹¹ are each independently alkyl, or one of R¹⁰ and R¹¹ is hydrogen, and the other is alkyl;
R¹⁰ and R¹¹ together with the nitrogen atom to which they are attached form a saturated heterocyclyl optionally containing one or more additional heteroatoms selected from N, O and S and optionally substituted with one or more R¹⁴;
R¹⁴ is selected from the group consisting of halogen, cyano, sulfonyl, alkyl, haloalkyl, alkylalkoxyl, a saturated or unsaturated heterocyclyl, —NR¹⁵R¹⁶, and —OR¹⁷;
R¹⁵ and R¹⁶ are each independently alkyl, or one of R¹⁵ and R¹⁶ is hydrogen, and the other is alkyl, and
R¹⁷ is selected from alkyl or haloalkyl.

In certain embodiments, L is ethyl or propyl.

In certain embodiments, R⁵ is hydrogen, methyl or trifluoromethyl.

In certain embodiments, R¹⁰ and R¹¹ together with the nitrogen atom to which they are attached form a saturated heterocyclyl selected from the group consisting of:

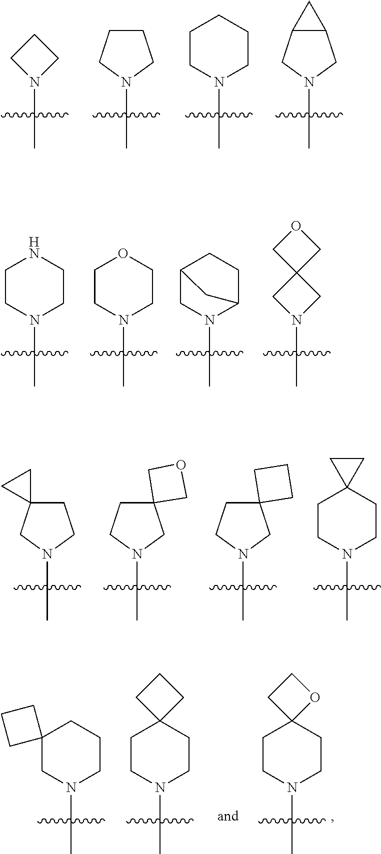

each of which is optionally substituted with one or more R¹⁴.

In certain embodiments, each R¹⁴ is independently selected from the group consisting of halogen, cyano, sulfonyl, alkyl, haloalkyl, alkylalkoxyl, —NR¹⁵R¹⁶, and OR¹⁷.

In certain embodiments, R¹⁵ and R¹⁶ are each independently alkyl. In certain embodiments, R¹⁵ and R¹⁶ are each independently C₁₋₆ alkyl, C₁₋₅ alkyl, C₁₋₄ alkyl, or C₁₋₃ alkyl. In certain embodiments, R¹⁵ and R¹⁶ are both C₁₋₃ alkyl. In certain embodiments, R¹⁵ and R¹⁶ are both methyl.

In certain embodiments, one of R¹⁵ and R¹⁶ is hydrogen, and the other is alkyl. In certain embodiments, one of R¹⁵ and R¹⁶ is hydrogen, and the other is C₁₋₆ alkyl, C₁₋₅ alkyl, C₁₋₄ alkyl, or C₁₋₃ alkyl. In certain embodiments, one of R¹⁵ and R¹⁶ is hydrogen, and the other is methyl.

In certain embodiments, R¹⁷ is selected from alkyl or haloalkyl.

In another aspect, the present disclosure provides compounds selected from the group consisting of:

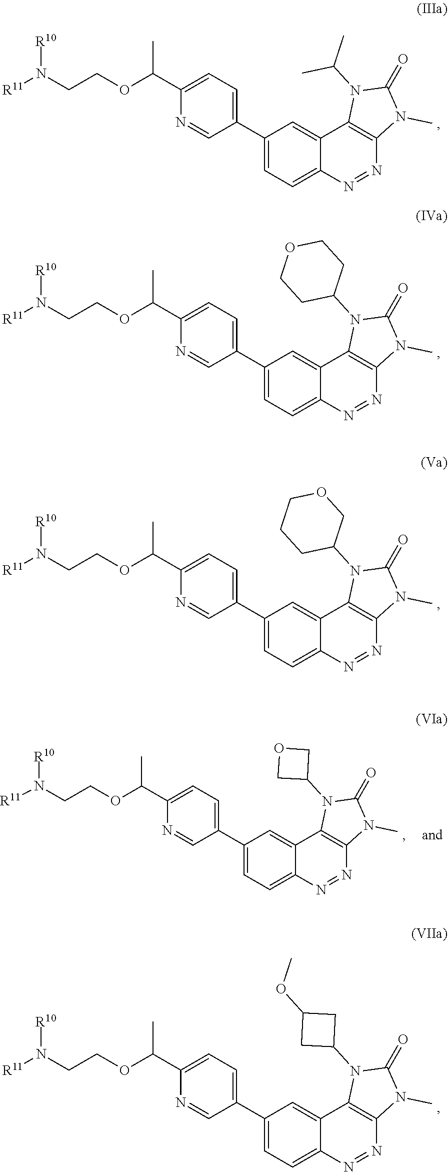

wherein R¹⁰ and R¹¹ are as defined as supra.

In certain embodiments, in any of Formula (IIIa) to (VIIa), R¹⁰ and R¹¹ together with the nitrogen atom to which they are attached form a saturated heterocyclyl selected from the group consisting of:

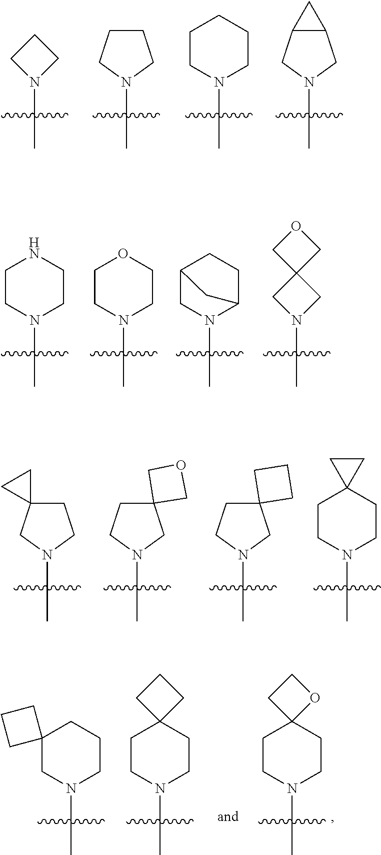

each of which is optionally substituted with one or more R¹⁴.

In a further aspect, the present disclosure provides compounds selected from the group consisting of:

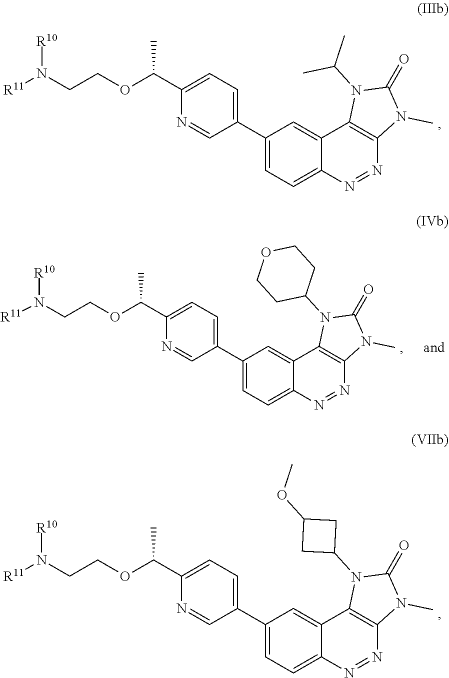

wherein R¹⁰ and R¹¹ are as defined as supra.

In certain embodiments, in any of Formula (IIIb), (IVb) and (VIIb), R¹⁰ and R¹¹ together with the nitrogen atom to which they are attached form a saturated heterocyclyl selected from the group consisting of:

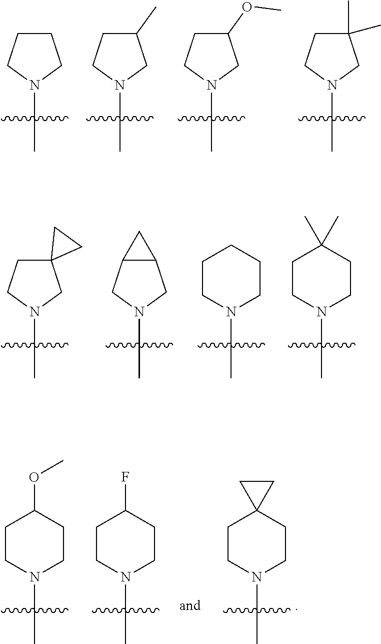

In one aspect, the present disclosure provides a compound of Formula (I) or a pharmaceutically acceptable salt thereof selected from the group consisting of:
8-(6-((2-(3-azabicyclo[3.1.0]hexan-3-yl)ethoxy)methyl)pyridin-3-yl)-1-isopropyl-3-methyl-1H-imidazo[4,5-c]cinnolin-2(3H)-one
8-(6-((2-(dimethylamino)ethoxy)methyl)pyridin-3-yl)-1-isopropyl-3-methyl-1H-imidazo[4,5-c]cinnolin-2(3H)-one
1-isopropyl-3-methyl-8-(6-((2-(piperidin-1-yl)ethoxy)methyl) pyridin-3-yl)-1H-imidazo[4,5-c]cinnolin-2(3H)-one
1-isopropyl-3-methyl-8-(6-((2-(pyrrolidin-1-yl)ethoxy)methyl)pyridin-3-yl)-1H-imidazo[4,5-c]cinnolin-2(3H)-one
8-(6-((2-(4-fluoropiperidin-1-yl)ethoxy)methyl)pyridin-3-yl)-1-isopropyl-3-methyl-1H-imidazo[4,5-c]cinnolin-2(3H)-one
1-isopropyl-8-(6-((2-methoxyethoxy)methyl)pyridin-3-yl)-3-methyl-1H-imidazo[4,5-c]cinnolin-2(3H)-one
1-isopropyl-3-methyl-8-(6-(((1-methylpiperidin-4-yl)oxy)methyl) pyridin-3-yl)-1H-imidazo[4,5-c]cinnolin-2(3H)-one
8-(6-((3-(dimethylamino)propoxy)methyl)pyridin-3-yl)-1-isopropyl-3-methyl-1H-imidazo[4,5-c]cinnolin-2(3H)-one
1-isopropyl-3-methyl-8-(6-(((1-methylpiperidin-4-yl)methoxy)methyl)pyridin-3-yl)-1H-imidazo[4,5-c]cinnolin-2(3H)-one
1-isopropyl-3-methyl-8-(6-(((1-methylpyrrolidin-2-yl)methoxy) methyl)pyridin-3-yl)-1H-imidazo[4,5-c]cinnolin-2(3H)-one
1-isopropyl-3-methyl-8-(6-(((1-methylpyrrolidin-3-yl)oxy) methyl)pyridin-3-yl)-1H-imidazo[4,5-c]cinnolin-2(3H)-one
8-(6-((3-(3-azabicyclo[3.1.0]hexan-3-yl)propoxy)methyl)pyridin-3-yl)-1-isopropyl-3-methyl-1H-imidazo[4,5-c]cinnolin-2(3H)-one
1-isopropyl-3-methyl-8-(6-(((1-methylpiperidin-3-yl)oxy)methyl) pyridin-3-yl)-1H-imidazo[4,5-c]cinnolin-2(3H)-one
8-(6-((3-(dimethylamino)cyclobutoxy)methyl)pyridin-3-yl)-1-isopropyl-3-methyl-1H-imidazo[4,5-c]cinnolin-2(3H)-one
1-isopropyl-3-methyl-8-(6-(((1-methylazetidin-3-yl)oxy)methyl) pyridin-3-yl)-1H-imidazo[4,5-c]cinnolin-2(3H)-one
8-(6-((2-(3-azabicyclo[3.1.0]hexan-3-yl)propoxy)methyl)pyridin-3-yl)-1-isopropyl-3-methyl-1H-imidazo[4,5-c]cinnolin-2(3H)-one
8-(6-((2-(3-azabicyclo[3.1.0]hexan-3-yl)-2-methylpropoxy) methyl)pyridin-3-yl)-1-isopropyl-3-methyl-1H-imidazo[4,5-c]cinnolin-2(3H)-one
8-(6-(((1-(3-azabicyclo[3.1.0]hexan-3-yl)cyclopropyl) methoxy)methyl)pyridin-3-yl)-1-isopropyl-3-methyl-1H-imidazo[4,5-c]cinnolin-2(3H)-one
1-isopropyl-3-methyl-8-(6-(((1-methylpiperidin-3-yl)methoxy)methyl)pyridin-3-yl)-1H-imidazo[4,5-c]cinnolin-2(3H)-one
1-isopropyl-3-methyl-8-(6-(((1-methylpyrrolidin-3-yl)methoxy)methyl)pyridin-3-yl)-1H-imidazo[4,5-c]cinnolin-2(3H)-one
8-(6-(((1-(3-azabicyclo[3.1.0]hexan-3-yl)propan-2-yl)oxy)methyl)pyridin-3-yl)-1-isopropyl-3-methyl-1H-imidazo[4,5-c]cinnolin-2(3H)-one
8-(6-(((1-(3-azabicyclo[3.1.0]hexan-3-yl)-2-methylpropan-2-yl)oxy)methyl)pyridin-3-yl)-1-isopropyl-3-methyl-1H-imidazo[4,5-c]cinnolin-2(3H)-one
8-(6-((1-(3-azabicyclo[3.1.0]hexan-3-ylmethyl)cyclopropoxy) methyl)pyridin-3-yl)-1-isopropyl-3-methyl-1H-imidazo[4,5-c]cinnolin-2(3H)-one
1-(2-((5-(1-isopropyl-3-methyl-2-oxo-2,3-dihydro-1H-imidazo[4,5-c]cinnolin-8-yl)pyridin-2-yl)methoxy)ethyl)piperidine-4-carbonitrile
1-(2-((R)-1-(5-(1-isopropyl-3-methyl-2-oxo-2,3-dihydro-1H-imidazo[4,5-c]cinnolin-8-yl)pyridin-2-yl)ethoxy)ethyl)-3-methylpyrrolidine-3-carbonitrile
8-(6-((2-(5-azaspiro[2.4]heptan-5-yl)ethoxy)methyl)pyridin-3-yl)-1-isopropyl-3-methyl-1H-imidazo[4,5-c]cinnolin-2(3H)-one
1-isopropyl-8-(6-((2-(4-methoxypiperidin-1-yl)ethoxy)methyl) pyridin-3-yl)-3-methyl-1H-imidazo[4,5-c]cinnolin-2(3H)-one
(R)-8-(6-((2-(3-fluoropyrrolidin-1-yl)ethoxy)methyl)pyridin-3-yl)-1-isopropyl-3-methyl-1H-imidazo[4,5-c]cinnolin-2(3H)-one
(S)-1-isopropyl-3-methyl-8-(6-((2-(2-methylpyrrolidin-1-yl)ethoxy)methyl)pyridin-3-yl)-1H-imidazo[4,5-c]cinnolin-2(3H)-one
8-(6-((2-(4,4-difluoropiperidin-1-yl)ethoxy)methyl)pyridin-3-yl)-1-isopropyl-3-methyl-1H-imidazo[4,5-c]cinnolin-2(3H)-one
8-(6-((2-(4,4-dimethylpiperidin-1-yl)ethoxy)methyl)pyridin-3-yl)-1-isopropyl-3-methyl-1H-imidazo[4,5-c]cinnolin-2(3H)-one
1-isopropyl-3-methyl-8-(6-((2-(4-methylpiperazin-1-yl)ethoxy) methyl)pyridin-3-yl)-1H-imidazo[4,5-c]cinnolin-2(3H)-one
1-isopropyl-3-methyl-8-(6-((2-morpholinoethoxy)methyl) pyridin-3-yl)-1H-imidazo[4,5-c]cinnolin-2(3H)-one
(S)-8-(6-((2-(3-fluoropyrrolidin-1-yl)ethoxy)methyl)pyridin-3-yl)-1-isopropyl-3-methyl-1H-imidazo[4,5-c]cinnolin-2(3H)-one
(R)-1-isopropyl-3-methyl-8-(6-((2-(2-methylpyrrolidin-1-yl)ethoxy)methyl)pyridin-3-yl)-1H-imidazo[4,5-c]cinnolin-2(3H)-one
8-(6-((2-(3,3-dimethylpyrrolidin-1-yl)ethoxy)methyl)pyridin-3-yl)-1-isopropyl-3-methyl-1H-imidazo[4,5-c]cinnolin-2(3H)-one
8-(6-((2-(3-(dimethylamino)azetidin-1-yl)ethoxy)methyl)pyridin-3-yl)-1-isopropyl-3-methyl-1H-imidazo[4,5-c]cinnolin-2(3H)-one
8-(6-((2-(3-fluoropiperidin-1-yl)ethoxy)methyl)pyridin-3-yl)-1-isopropyl-3-methyl-1H-imidazo[4,5-c]cinnolin-2(3H)-one
8-(6-((2-(2-azabicyclo[2.2.1]heptan-2-yl)ethoxy)methyl)pyridin-3-yl)-1-isopropyl-3-methyl-1H-imidazo[4,5-c]cinnolin-2(3H)-one
8-(6-((2-(6-azaspiro[3.5]nonan-6-yl)ethoxy)methyl)pyridin-3-yl)-1-isopropyl-3-methyl-1H-imidazo[4,5-c]cinnolin-2(3H)-one
1-isopropyl-8-(6-((2-(3-methoxypiperidin-1-yl)ethoxy)methyl) pyridin-3-yl)-3-methyl-1H-imidazo[4,5-c]cinnolin-2(3H)-one
(S)-8-(6-((2-(3-(dimethylamino)pyrrolidin-1-yl)ethoxy)methyl) pyridin-3-yl)-1-isopropyl-3-methyl-1H-imidazo[4,5-c]cinnolin-2(3H)-one
(S)-1-isopropyl-8-(6-((2-(3-methoxypyrrolidin-1-yl)ethoxy)methyl)pyridin-3-yl)-3-methyl-1H-imidazo[4,5-c]cinnolin-2(3H)-one
1-isopropyl-8-(6-((2-(4-methoxypiperidin-1-yl)ethoxy)methyl) pyridin-3-yl)-3-methyl-1H-imidazo[4,5-c]cinnolin-2(3H)-one
8-(6-((2-((2R,5S)-2,5-dimethylpyrrolidin-1-yl)ethoxy)methyl) pyridin-3-yl)-1-isopropyl-3-methyl-1H-imidazo[4,5-c]cinnolin-2(3H)-one
(R)-8-(6-((2-(3-(dimethylamino)pyrrolidin-1-yl)ethoxy)methyl) pyridin-3-yl)-1-isopropyl-3-methyl-1H-imidazo[4,5-c]cinnolin-2(3H)-one
1-isopropyl-3-methyl-8-(6-((2-(4-(trifluoromethoxy)piperidin-1-yl)ethoxy)methyl)pyridin-3-yl)-1H-imidazo[4,5-c]cinnolin-2(3H)-one
8-(6-((2-(6,6-difluoro-3-azabicyclo[3.1.0]hexan-3-yl)ethoxy) methyl)pyridin-3-yl)-1-isopropyl-3-methyl-1H-imidazo[4,5-c]cinnolin-2(3H)-one
8-(6-((2-(7-azaspiro[3.5]nonan-7-yl)ethoxy)methyl)pyridin-3-yl)-1-isopropyl-3-methyl-1H-imidazo[4,5-c]cinnolin-2(3H)-one
(R)-8-(6-((2-(3-(difluoromethoxy)pyrrolidin-1-yl)ethoxy) methyl)pyridin-3-yl)-1-isopropyl-3-methyl-1H-imidazo[4,5-c]cinnolin-2(3H)-one
(S)-8-(6-((2-(3-(difluoromethoxy)pyrrolidin-1-yl)ethoxy) methyl)pyridin-3-yl)-1-isopropyl-3-methyl-1H-imidazo[4,5-c]cinnolin-2(3H)-one
1-isopropyl-3-methyl-8-(6-((2-(3-(trifluoromethoxy)pyrrolidin-1-yl)ethoxy)methyl)pyridin-3-yl)-1,3-dihydro-2H-imidazo[4,5-c]cinnolin-2-one
(R)-8-(6-(1-(2-(5-azaspiro[2.4]heptan-5-yl)ethoxy)ethyl)pyridin-3-yl)-1-isopropyl-3-methyl-1H-imidazo[4,5-c]cinnolin-2(3H)-one
(R)-8-(6-(1-(2-(6-azaspiro[2.5]octan-6-yl)ethoxy)ethyl)pyridin-3-yl)-1-isopropyl-3-methyl-1H-imidazo[4,5-c]cinnolin-2(3H)-one
(R)-8-(6-(1-(2-(6-azaspiro[2.5]octan-6-yl)ethoxy)ethyl)pyridin-3-yl)-1-isopropyl-3-methyl-1H-imidazo[4,5-c]cinnolin-2(3H)-one
(R)-1-isopropyl-8-(6-(1-(2-(4-methoxypiperidin-1-yl)ethoxy)ethyl)pyridin-3-yl)-3-methyl-1H-imidazo[4,5-c]cinnolin-2(3H)-one
1-isopropyl-8-(6-((R)-1-(2-((R)-3-methoxypyrrolidin-1-yl)ethoxy)ethyl)pyridin-3-yl)-3-methyl-1H-imidazo[4,5-c]cinnolin-2(3H)-one
(R)-1-isopropyl-3-methyl-8-(6-(1-(2-(4-(trifluoromethoxy)piperidin-1-yl)ethoxy)ethyl)pyridin-3-yl)-1H-imidazo[4,5-c]cinnolin-2(3H)-one
1-isopropyl-8-(6-((R)-1-(2-((S)-3-methoxypyrrolidin-1-yl)ethoxy)ethyl)pyridin-3-yl)-3-methyl-1H-imidazo[4,5-c]cinnolin-2(3H)-one
8-(6-((2-(3,3-dimethylpyrrolidin-1-yl)ethoxy)methyl)pyridin-3-yl)-7-fluoro-1-isopropyl-3-methyl-1H-imidazo[4,5-c]cinnolin-2(3H)-one
(R)-8-(6-(1-(2-(3,3-dimethylpyrrolidin-1-yl)ethoxy)ethyl)pyridin-3-yl)-1-isopropyl-3-methyl-1H-imidazo[4,5-c]cinnolin-2(3H)-one
(R)-1-isopropyl-3-methyl-8-(6-(1-(2-(pyrrolidin-1-yl)ethoxy)ethyl)pyridin-3-yl)-1,3-dihydro-2H-imidazo[4,5-c]cinnolin-2-one
8-(6-((R)-1-(2-((S)-3-(difluoromethoxy)pyrrolidin-1-yl)ethoxy)ethyl)pyridin-3-yl)-1-isopropyl-3-methyl-1H-imidazo[4,5-c]cinnolin-2(3H)-one
1-isopropyl-8-(6-((S)-1-(2-((R)-3-methoxypyrrolidin-1-yl)ethoxy)ethyl)pyridin-3-yl)-3-methyl-1H-imidazo[4,5-c]cinnolin-2(3H)-one
1-isopropyl-8-(6-((S)-1-(2-((S)-3-methoxypyrrolidin-1-yl)ethoxy)ethyl)pyridin-3-yl)-3-methyl-1H-imidazo[4,5-c]cinnolin-2(3H)-one
8-(6-((1S)-1-(2-(3-azabicyclo[3.1.0]hexan-3-yl)ethoxy)ethyl) pyridin-3-yl)-1-isopropyl-3-methyl-1H-imidazo[4,5-c]cinnolin-2(3H)-one
8-(6-((1R)-1-(2-(3-azabicyclo[3.1.0]hexan-3-yl)ethoxy)ethyl) pyridin-3-yl)-1-isopropyl-3-methyl-1H-imidazo[4,5-c]cinnolin-2(3H)-one
(S)-8-(6-(1-(2-(4-fluoropiperidin-1-yl)ethoxy)ethyl)pyridin-3-yl)-1-isopropyl-3-methyl-1H-imidazo[4,5-c]cinnolin-2(3H)-one
(R)-8-(6-(1-(2-(4-fluoropiperidin-1-yl)ethoxy)ethyl)pyridin-3-yl)-1-isopropyl-3-methyl-1H-imidazo[4,5-c]cinnolin-2(3H)-one
8-(6-((1S)-1-(2-(6,6-difluoro-3-azabicyclo[3.1.0]hexan-3-yl)ethoxy)ethyl)pyridin-3-yl)-1-isopropyl-3-methyl-1H-imidazo[4,5-c]cinnolin-2(3H)-one
8-(6-((1R)-1-(2-(6,6-difluoro-3-azabicyclo[3.1.0]hexan-3-yl)ethoxy)ethyl)pyridin-3-yl)-1-isopropyl-3-methyl-1H-imidazo[4,5-c]cinnolin-2(3H)-one
(S)-8-(6-(1-(2-(4,4-dimethylpiperidin-1-yl)ethoxy)ethyl)pyridin-3-yl)-1-isopropyl-3-methyl-1H-imidazo[4,5-c]cinnolin-2(3H)-one
(R)-8-(6-(1-(2-(4,4-dimethylpiperidin-1-yl)ethoxy)ethyl)pyridin-3-yl)-1-isopropyl-3-methyl-1H-imidazo[4,5-c]cinnolin-2(3H)-one
(S)-8-(6-(1-(2-(4,4-difluoropiperidin-1-yl)ethoxy)ethyl)pyridin-3-yl)-1-isopropyl-3-methyl-1H-imidazo[4,5-c]cinnolin-2(3H)-one
(R)-8-(6-(1-(2-(4,4-difluoropiperidin-1-yl)ethoxy)ethyl)pyridin-3-yl)-1-isopropyl-3-methyl-1H-imidazo[4,5-c]cinnolin-2(3H)-one
8-(6-((R)-1-(2-((R)-3-cyclopropoxypyrrolidin-1-yl)ethoxy)ethyl) pyridin-3-yl)-1-isopropyl-3-methyl-1H-imidazo[4,5-c]cinnolin-2(3H)-one
1-isopropyl-3-methyl-8-(6-((R)-1-(2-((R)-3-methylpyrrolidin-1-yl)ethoxy)ethyl)pyridin-3-yl)-1H-imidazo[4,5-c]cinnolin-2(3H)-one
1-isopropyl-3-methyl-8-(6-((R)-1-(2-((S)-3-methylpyrrolidin-1-yl)ethoxy)ethyl)pyridin-3-yl)-1H-imidazo[4,5-c]cinnolin-2(3H)-one
1-isopropyl-3-methyl-8-(6-((R)-1-(2-((R)-3-(methylsulfonyl)pyrrolidin-1-yl)ethoxy)ethyl)pyridin-3-yl)-1H-imidazo[4,5-c]cinnolin-2(3H)-one
8-(6-((R)-1-(2-((R)-3-cyclopropoxypyrrolidin-1-yl)ethoxy)ethyl) pyridin-3-yl)-3-methyl-1-(tetrahydro-2H-pyran-4-yl)-1H-imidazo[4,5-c]cinnolin-2(3H)-one
(R)-8-(6-(1-(2-(2-oxa-6-azaspiro[3.3]heptan-6-yl)ethoxy)ethyl) pyridin-3-yl)-1-isopropyl-3-methyl-1H-imidazo[4,5-c]cinnolin-2(3H)-one
(R)-8-(6-(1-(2-(1-oxa-7-azaspiro[3.5]nonan-7-yl)ethoxy)ethyl) pyridin-3-yl)-1-isopropyl-3-methyl-1H-imidazo[4,5-c]cinnolin-2(3H)-one
(R)-1-isopropyl-3-methyl-8-(6-(1-(2-(4-(oxetan-3-yl)piperidin-1-yl)ethoxy)ethyl)pyridin-3-yl)-1H-imidazo[4,5-c]cinnolin-2(3H)-one
(R)-1-isopropyl-3-methyl-8-(6-(1-(2-(4-(trifluoromethyl) piperidin-1-yl)ethoxy)ethyl)pyridin-3-yl)-1H-imidazo[4,5-c]cinnolin-2(3H)-one
1-isopropyl-8-(6-((R)-1-(2-((R)-3-(methoxymethyl)pyrrolidin-1-yl)ethoxy)ethyl)pyridin-3-yl)-3-methyl-1H-imidazo[4,5-c]cinnolin-2(3H)-one
(R)-8-(6-(1-(2-(2-oxa-6-azaspiro[3.4]octan-6-yl)ethoxy)ethyl) pyridin-3-yl)-1-isopropyl-3-methyl-1H-imidazo[4,5-c]cinnolin-2(3H)-one
(R)-1-isopropyl-3-methyl-8-(6-(1-(2-(piperidin-1-yl)ethoxy)ethyl) pyridin-3-yl)-1H-imidazo[4,5-c]cinnolin-2(3H)-one
1-isopropyl-3-methyl-8-(6-((R)-1-(2-((R)-3-(trifluoromethoxy) pyrrolidin-1-yl)ethoxy)ethyl)pyridin-3-yl)-1H-imidazo[4,5-c]cinnolin-2(3H)-one
(R)-8-(6-(1-(2-(6-azaspiro[3.4]octan-6-yl)ethoxy)ethyl)pyridin-3-yl)-1-isopropyl-3-methyl-1H-imidazo[4,5-c]cinnolin-2(3H)-one
1-(2-((R)-1-(5-(1-isopropyl-3-methyl-2-oxo-2,3-dihydro-1H-imidazo[4,5-c]cinnolin-8-yl)pyridin-2-yl)ethoxy)ethyl)-3-methylpyrrolidine-3-carbonitrile
(R)-1-(2-(1-(5-(1-isopropyl-3-methyl-2-oxo-2,3-dihydro-1H-imidazo[4,5-c]cinnolin-8-yl)pyridin-2-yl)ethoxy)ethyl)piperidine-4-carbonitrile
(R)-8-(6-(1-(2-(4-isopropoxypiperidin-1-yl)ethoxy)ethyl)pyridin-3-yl)-1-isopropyl-3-methyl-1H-imidazo[4,5-c]cinnolin-2(3H)-one
8-(6-((R)-1-(2-((R)-3-isopropoxypyrrolidin-1-yl)ethoxy) ethyl)pyridin-3-yl)-1-isopropyl-3-methyl-1H-imidazo[4,5-c]cinnolin-2(3H)-one
8-(6-((2-(5-azaspiro[2.4]heptan-5-yl)ethoxy)methyl)pyridin-3-yl)-3-methyl-1-(tetrahydro-2H-pyran-4-yl)-1H-imidazo[4,5-c]cinnolin-2(3H)-one
8-(6-((2-(4-fluoropiperidin-1-yl)ethoxy)methyl)pyridin-3-yl)-3-methyl-1-(tetrahydro-2H-pyran-4-yl)-1H-imidazo[4,5-c]cinnolin-2(3H)-one
8-(6-((2-(4,4-dimethylpiperidin-1-yl)ethoxy)methyl)pyridin-3-yl)-3-methyl-1-(tetrahydro-2H-pyran-4-yl)-1H-imidazo[4,5-c]cinnolin-2(3H)-one
8-(6-((2-(3,3-dimethylpyrrolidin-1-yl)ethoxy)methyl)pyridin-3-yl)-3-methyl-1-(tetrahydro-2H-pyran-4-yl)-1H-imidazo[4,5-c]cinnolin-2(3H)-one
8-(6-((2-(3-azabicyclo[3.1.0]hexan-3-yl)ethoxy)methyl)pyridin-3-yl)-3-methyl-1-(tetrahydro-2H-pyran-4-yl)-1H-imidazo[4,5-c]cinnolin-2(3H)-one
(R)-8-(6-((2-(3-(difluoromethoxy)pyrrolidin-1-yl)ethoxy)methyl) pyridin-3-yl)-3-methyl-1-(tetrahydro-2H-pyran-4-yl)-1H-imidazo[4,5-c]cinnolin-2(3H)-one
(S)-8-(6-((2-(3-(difluoromethoxy)pyrrolidin-1-yl)ethoxy)methyl)pyridin-3-yl)-3-methyl-1-(tetrahydro-2H-pyran-4-yl)-1H-imidazo[4,5-c]cinnolin-2(3H)-one
3-methyl-1-(tetrahydro-2H-pyran-4-yl)-8-(6-((2-(4-(trifluoromethoxy)piperidin-1-yl)ethoxy)methyl)pyridin-3-yl)-1H-imidazo[4,5-c]cinnolin-2(3H)-one
(R)-8-(6-(1-(2-(5-azaspiro[2.4]heptan-5-yl)ethoxy)ethyl)pyridin-3-yl)-3-methyl-1-(tetrahydro-2H-pyran-4-yl)-1H-imidazo[4,5-c]cinnolin-2(3H)-one
(R)-8-(6-(1-(2-(4,4-dimethylpiperidin-1-yl)ethoxy)ethyl)pyridin-3-yl)-3-methyl-1-(tetrahydro-2H-pyran-4-yl)-1H-imidazo[4,5-c]cinnolin-2(3H)-one
8-(6-((1R)-1-(2-(3-azabicyclo[3.1.0]hexan-3-yl)ethoxy)ethyl)pyridin-3-yl)-3-methyl-1-(tetrahydro-2H-pyran-4-yl)-1H-imidazo[4,5-c]cinnolin-2(3H)-one
8-(6-((R)-1-(2-((S)-3-methoxypyrrolidin-1-yl)ethoxy)ethyl)pyridin-3-yl)-3-methyl-1-(tetrahydro-2H-pyran-4-yl)-1H-imidazo[4,5-c]cinnolin-2(3H)-one
3-methyl-1-(tetrahydro-2H-pyran-4-yl)-8-(6-((1R)-1-(2-(3-(trifluoromethoxy)pyrrolidin-1-yl)ethoxy)ethyl)pyridin-3-yl)-1H-imidazo[4,5-c]cinnolin-2(3H)-one
(R)-8-(6-(1-(2-(6-azaspiro[2.5]octan-6-yl)ethoxy)ethyl)pyridin-3-yl)-3-methyl-1-(tetrahydro-2H-pyran-4-yl)-1H-imidazo[4,5-c]cinnolin-2(3H)-one
8-(6-((R)-1-(2-(4-fluoropiperidin-1-yl)ethoxy)ethyl)pyridin-3-yl)-3-methyl-1-(tetrahydro-2H-pyran-4-yl)-1,3,3 a,9b-tetrahydro-2H-imidazo[4,5-c]cinnolin-2-one
(R)-3-methyl-1-(tetrahydro-2H-pyran-4-yl)-8-(6-(1-(2-(4-(trifluoromethoxy)piperidin-1-yl)ethoxy)ethyl)pyridin-3-yl)-1H-imidazo[4,5-c]cinnolin-2(3H)-one
(R)-8-(6-(1-(2-(4-methoxypiperidin-1-yl)ethoxy)ethyl)pyridin-3-yl)-3-methyl-1-(tetrahydro-2H-pyran-4-yl)-1H-imidazo[4,5-c]cinnolin-2(3H)-one
(R)-8-(6-(1-(2-(3,3-dimethylpyrrolidin-1-yl)ethoxy)ethyl)pyridin-3-yl)-3-methyl-1-(tetrahydro-2H-pyran-4-yl)-1H-imidazo[4,5-c]cinnolin-2(3H)-one
8-(6-((R)-1-(2-((R)-3-methoxypyrrolidin-1-yl)ethoxy)ethyl) pyridin-3-yl)-3-methyl-1-(tetrahydro-2H-pyran-4-yl)-1H-imidazo[4,5-c]cinnolin-2(3H)-one
8-(6-((R)-1-(2-((R)-3-isopropoxypyrrolidin-1-yl)ethoxy)ethyl)pyridin-3-yl)-3-methyl-1-(tetrahydro-2H-pyran-4-yl)-1H-imidazo[4,5-c]cinnolin-2(3H)-one
(R)-1-(2-(1-(5-(3-methyl-2-oxo-1-(tetrahydro-2H-pyran-4-yl)-2,3-dihydro-1H-imidazo[4,5-c]cinnolin-8-yl)pyridin-2-yl)ethoxy)ethyl)piperidine-4-carbonitrile
8-(6-((R)-1-(2-((S)-3-(difluoromethoxy)pyrrolidin-1-yl)ethoxy)ethyl)pyridin-3-yl)-3-methyl-1-(tetrahydro-2H-pyran-4-yl)-1,3-dihydro-2H-imidazo[4,5-c]cinnolin-2-one (R)-8-(6-(1-(2-(2-oxa-6-azaspiro[3.4]octan-6-yl)ethoxy)ethyl) pyridin-3-yl)-3-methyl-1-(tetrahydro-2H-pyran-4-yl)-1H-imidazo[4,5-c]cinnolin-2(3H)-one
(R)-3-methyl-1-(tetrahydro-2H-pyran-4-yl)-8-(6-(1-(2-(4-(trifluoromethyl)piperidin-1-yl)ethoxy)ethyl)pyridin-3-yl)-1H-imidazo[4,5-c]cinnolin-2(3H)-one
(R)-8-(6-(1-(2-(6-azaspiro[3.4]octan-6-yl)ethoxy)ethyl)pyridin-3-yl)-3-methyl-1-(tetrahydro-2H-pyran-4-yl)-1H-imidazo[4,5-c]cinnolin-2(3H)-one
8-(6-((R)-1-(2-((R)-3-(methoxymethyl)pyrrolidin-1-yl)ethoxy) ethyl)pyridin-3-yl)-3-methyl-1-(tetrahydro-2H-pyran-4-yl)-1H-imidazo[4,5-c]cinnolin-2(3H)-one
(R)-3-methyl-8-(6-(1-(2-(piperidin-1-yl)ethoxy)ethyl)pyridin-3-yl)-1-(tetrahydro-2H-pyran-4-yl)-1H-imidazo[4,5-c]cinnolin-2(3H)-one
(R)-3-methyl-8-(6-(1-(2-(pyrrolidin-1-yl)ethoxy)ethyl)pyridin-3-yl)-1-(tetrahydro-2H-pyran-4-yl)-1H-imidazo[4,5-c]cinnolin-2(3H)-one
8-(6-((R)-1-(2-((R)-3-cyclopropoxypyrrolidin-1-yl)ethoxy)ethyl) pyridin-3-yl)-3-methyl-1-(tetrahydro-2H-pyran-4-yl)-1H-imidazo[4,5-c]cinnolin-2(3H)-one
3-methyl-8-(6-((R)-1-(2-((R)-3-methylpyrrolidin-1-yl)ethoxy)ethyl)pyridin-3-yl)-1-(tetrahydro-2H-pyran-4-yl)-1H-imidazo[4,5-c]cinnolin-2(3H)-one
(R)-8-(6-(1-(2-(7-azaspiro[3.5]nonan-7-yl)ethoxy)ethyl)pyridin-3-yl)-3-methyl-1-(tetrahydro-2H-pyran-4-yl)-1H-imidazo[4,5-c]cinnolin-2(3H)-one
(R)-8-(6-(1-(2-(1-oxa-7-azaspiro[3.5]nonan-7-yl)ethoxy)ethyl)pyridin-3-yl)-3-methyl-1-(tetrahydro-2H-pyran-4-yl)-1H-imidazo[4,5-c]cinnolin-2(3H)-one
3-methyl-8-(6-((R)-1-(2-((S)-3-methylpyrrolidin-1-yl)ethoxy)ethyl)pyridin-3-yl)-1-(tetrahydro-2H-pyran-4-yl)-1H-imidazo[4,5-c]cinnolin-2(3H)-one
(R)-8-(6-(1-(2-(2-oxa-6-azaspiro[3.3]heptan-6-yl)ethoxy)ethyl)pyridin-3-yl)-3-methyl-1-(tetrahydro-2H-pyran-4-yl)-1H-imidazo[4,5-c]cinnolin-2(3H)-one
(R)-3-methyl-8-(6-(1-(2-(4-(oxetan-3-yl)piperidin-1-yl)ethoxy)ethyl)pyridin-3-yl)-1-(tetrahydro-2H-pyran-4-yl)-1H-imidazo[4,5-c]cinnolin-2(3H)-one
3-methyl-8-(6-((R)-1-(2-((R)-3-(methylsulfonyl)pyrrolidin-1-yl)ethoxy)ethyl)pyridin-3-yl)-1-(tetrahydro-2H-pyran-4-yl)-1H-imidazo[4,5-c]cinnolin-2(3H)-one
3-methyl-1-(2-((R)-1-(5-(3-methyl-2-oxo-1-(tetrahydro-2H-pyran-4-yl)-2,3-dihydro-1H-imidazo[4,5-c]cinnolin-8-yl)pyridin-2-yl)ethoxy)ethyl) pyrrolidine-3-carbonitrile
8-(6-((2-(5-azaspiro[2.4]heptan-5-yl)ethoxy)methyl)pyridin-3-yl)-7-fluoro-1-isopropyl-3-methyl-1H-imidazo[4,5-c]cinnolin-2(3H)-one
8-(6-((2-(3,3-dimethylpyrrolidin-1-yl)ethoxy)methyl)pyridin-3-yl)-7-fluoro-1-isopropyl-3-methyl-1H-imidazo[4,5-c]cinnolin-2(3H)-one
8-(6-((2-(4,4-dimethylpiperidin-1-yl)ethoxy)methyl)pyridin-3-yl)-7-fluoro-1-isopropyl-3-methyl-1H-imidazo[4,5-c]cinnolin-2(3H)-one
7-fluoro-1-isopropyl-3-methyl-8-(6-((2-(3-(trifluoromethoxy)pyrrolidin-1-yl)ethoxy)methyl)pyridin-3-yl)-1,3-dihydro-2H-imidazo[4,5-c]cinnolin-2-one
(R)-8-(6-(1-(2-(5-azaspiro[2.4]heptan-5-yl)ethoxy)ethyl)pyridin-3-yl)-7-fluoro-3-methyl-1-(tetrahydro-2H-pyran-4-yl)-1H-imidazo[4,5-c]cinnolin-2(3H)-one
(R)-7-fluoro-8-(6-(1-(2-(4-fluoropiperidin-1-yl)ethoxy)ethyl)pyridin-3-yl)-1-isopropyl-3-methyl-1,3-dihydro-2H-imidazo[4,5-c]cinnolin-2-one
(R)-8-(6-(1-(2-(3,3-dimethylpyrrolidin-1-yl)ethoxy)ethyl)pyridin-3-yl)-7-fluoro-1-isopropyl-3-methyl-1H-imidazo[4,5-c]cinnolin-2(3H)-one
7-fluoro-1-isopropyl-8-(6-((R)-1-(2-((S)-3-methoxypyrrolidin-1-yl)ethoxy)ethyl)pyridin-3-yl)-3-methyl-1H-imidazo[4,5-c]cinnolin-2(3H)-one
(R)-8-(6-(1-(2-(4,4-dimethylpiperidin-1-yl)ethoxy)ethyl)pyridin-3-yl)-7-fluoro-1-isopropyl-3-methyl-1H-imidazo[4,5-c]cinnolin-2(3H)-one
(R)-8-(6-(1-(2-(6-azaspiro[2.5]octan-6-yl)ethoxy)ethyl)pyridin-3-yl)-7-fluoro-1-isopropyl-3-methyl-1H-imidazo[4,5-c]cinnolin-2(3H)-one
7-fluoro-1-isopropyl-3-methyl-8-(6-((1R)-1-(2-(3-(trifluoromethoxy)pyrrolidin-1-yl)ethoxy)ethyl)pyridin-3-yl)-1H-imidazo[4,5-c]cinnolin-2(3H)-one
(R)-7-fluoro-1-isopropyl-8-(6-(1-(2-(4-methoxypiperidin-1-yl)ethoxy)ethyl)pyridin-3-yl)-3-methyl-1H-imidazo[4,5-c]cinnolin-2(3H)-one
8-(6-((1R)-1-(2-(3-azabicyclo[3.1.0]hexan-3-yl)ethoxy)ethyl)pyridin-3-yl)-7-fluoro-1-isopropyl-3-methyl-1H-imidazo[4,5-c]cinnolin-2(3H)-one
(R)-7-fluoro-1-isopropyl-3-methyl-8-(6-(1-(2-(4-(trifluoromethoxy)piperidin-1-yl)ethoxy)ethyl)pyridin-3-yl)-1H-imidazo[4,5-c]cinnolin-2(3H)-one
(R)-1-(2-(1-(5-(7-fluoro-1-isopropyl-3-methyl-2-oxo-2,3-dihydro-1H-imidazo[4,5-c]cinnolin-8-yl)pyridin-2-yl)ethoxy)ethyl)piperidine-4-carbonitrile
8-(6-((R)-1-(2-((R)-3-(difluoromethoxy)pyrrolidin-1-yl)ethoxy)ethyl)pyridin-3-yl)-7-fluoro-1-isopropyl-3-methyl-1H-imidazo[4,5-c]cinnolin-2(3H)-one
1-(2-((R)-1-(5-(7-fluoro-1-isopropyl-3-methyl-2-oxo-2,3-dihydro-1H-imidazo[4,5-c]cinnolin-8-yl)pyridin-2-yl)ethoxy)ethyl)-3-methylpyrrolidine-3-carbonitrile
(R)-8-(6-(1-(2-(5-azaspiro[2.4]heptan-5-yl)ethoxy)ethyl)pyridin-3-yl)-7-fluoro-3-methyl-1-(tetrahydro-2H-pyran-4-yl)-1H-imidazo[4,5-c]cinnolin-2(3H)-one
(R)-8-(6-(1-(2-(4,4-dimethylpiperidin-1-yl)ethoxy)ethyl)pyridin-3-yl)-7-fluoro-3-methyl-1-(tetrahydro-2H-pyran-4-yl)-1H-imidazo[4,5-c]cinnolin-2(3H)-one
(R)-7-fluoro-8-(6-(1-(2-(4-fluoropiperidin-1-yl)ethoxy)ethyl)pyridin-3-yl)-3-methyl-1-(tetrahydro-2H-pyran-4-yl)-1H-imidazo[4,5-c]cinnolin-2(3H)-one
7-fluoro-3-methyl-1-(tetrahydro-2H-pyran-4-yl)-8-(6-((R)-1-(2-((R)-3-(trifluoromethoxy)pyrrolidin-1-yl)ethoxy)ethyl)pyridin-3-yl)-1,3-dihydro-2H-imidazo[4,5-c]cinnolin-2-one
7-fluoro-3-methyl-1-(tetrahydro-2H-pyran-4-yl)-8-(6-((R)-1-(2-((S)-3-(trifluoromethoxy)pyrrolidin-1-yl)ethoxy)ethyl)pyridin-3-yl)-1,3-dihydro-2H-imidazo[4,5-c]cinnolin-2-one
(R)-8-(6-(1-(2-(3,3-dimethylpyrrolidin-1-yl)ethoxy)ethyl)pyridin-3-yl)-7-fluoro-3-methyl-1-(tetrahydro-2H-pyran-4-yl)-1H-imidazo[4,5-c]cinnolin-2(3H)-one
(R)-8-(6-(1-(2-(6-azaspiro[2.5]octan-6-yl)ethoxy)ethyl)pyridin-3-yl)-7-fluoro-3-methyl-1-(tetrahydro-2H-pyran-4-yl)-1H-imidazo[4,5-c]cinnolin-2(3H)-one
8-(6-((R)-1-(2-((R)-3-(difluoromethoxy)pyrrolidin-1-yl)ethoxy)ethyl)pyridin-3-yl)-7-fluoro-3-methyl-1-(tetrahydro-2H-pyran-4-yl)-1H-imidazo[4,5-c]cinnolin-2(3H)-one
(R)-8-(6-(1-(2-(7-azaspiro[3.5]nonan-7-yl)ethoxy)ethyl)pyridin-3-yl)-7-fluoro-3-methyl-1-(tetrahydro-2H-pyran-4-yl)-1H-imidazo[4,5-c]cinnolin-2(3H)-one
(R)-7-fluoro-3-methyl-1-(tetrahydro-2H-pyran-4-yl)-8-(6-(1-(2-(4-(trifluoromethoxy)piperidin-1-yl)ethoxy)ethyl)pyridin-3-yl)-1H-imidazo[4,5-c]cinnolin-2(3H)-one
8-(6-((R)-1-(2-((S)-3-(difluoromethoxy)pyrrolidin-1-yl)ethoxy)ethyl)pyridin-3-yl)-7-fluoro-3-methyl-1-(tetrahydro-2H-pyran-4-yl)-1,3-dihydro-2H-imidazo[4,5-c]cinnolin-2-one 8-(6-((2-(5-azaspiro[2.4]heptan-5-yl)ethoxy)methyl)pyridin-3-yl)-1-(trans-3-methoxycyclobutyl)-3-methyl-1H-imidazo[4,5-c]cinnolin-2(3H)-one
8-(6-((2-(3,3-dimethylpyrrolidin-1-yl)ethoxy)methyl)pyridin-3-yl)-1-(trans-3-methoxycyclobutyl)-3-methyl-1H-imidazo[4,5-c]cinnolin-2(3H)-one
8-(6-((R)-1-(2-(5-azaspiro[2.4]heptan-5-yl)ethoxy)ethyl)pyridin-3-yl)-1-(trans-3-methoxycyclobutyl)-3-methyl-1H-imidazo[4,5-c]cinnolin-2(3H)-one
1-(trans-3-methoxycyclobutyl)-3-methyl-8-(6-((R)-1-(2-(pyrrolidin-1-yl)ethoxy)ethyl)pyridin-3-yl)-1,3-dihydro-2H-imidazo[4,5-c]cinnolin-2-one
1-(trans-3-methoxycyclobutyl)-8-(6-((R)-1-(2-((R)-3-methoxypyrrolidin-1-yl)ethoxy)ethyl)pyridin-3-yl)-3-methyl-1H-imidazo[4,5-c]cinnolin-2(3H)-one
8-(6-((R)-1-(2-(6-azaspiro[2.5]octan-6-yl)ethoxy)ethyl)pyridin-3-yl)-1-(trans-3-methoxycyclobutyl)-3-methyl-1H-imidazo[4,5-c]cinnolin-2(3H)-one
8-(6-((2-(dimethylamino)ethoxy)methyl)pyridin-3-yl)-1-(trans-3-methoxycyclobutyl)-3-methyl-1H-imidazo[4,5-c]cinnolin-2(3H)-one
1-(trans-3-methoxycyclobutyl)-3-methyl-8-(6-((2-(pyrrolidin-1-yl)ethoxy)methyl)pyridin-3-yl)-1H-imidazo[4,5-c]cinnolin-2(3H)-one
1-(trans-3-methoxycyclobutyl)-3-methyl-8-(6-((2-(piperidin-1-yl)ethoxy)methyl)pyridin-3-yl)-1H-imidazo[4,5-c]cinnolin-2(3H)-one
8-(6-((2-(4-fluoropiperidin-1-yl)ethoxy)methyl)pyridin-3-yl)-1-(trans-3-methoxycyclobutyl)-3-methyl-1H-imidazo[4,5-c]cinnolin-2(3H)-one
8-(6-((2-(3-azabicyclo[3.1.0]hexan-3-yl)ethoxy)methyl)pyridin-3-yl)-1-(trans-3-methoxycyclobutyl)-3-methyl-1H-imidazo[4,5-c]cinnolin-2(3H)-one
8-(6-((R)-1-(2-(6-azaspiro[2.5]octan-6-yl)ethoxy)ethyl)pyridin-3-yl)-1-(cis-3-methoxycyclobutyl)-3-methyl-1H-imidazo[4,5-c]cinnolin-2(3H)-one
8-(6-((R)-1-(2-(5-azaspiro[2.4]heptan-5-yl)ethoxy)ethyl)pyridin-3-yl)-1-(cis-3-methoxycyclobutyl)-3-methyl-1H-imidazo[4,5-c]cinnolin-2(3H)-one
1-(cis-3-methoxycyclobutyl)-3-methyl-8-(6-((2-(pyrrolidin-1-yl)ethoxy)methyl)pyridin-3-yl)-1H-imidazo[4,5-c]cinnolin-2(3H)-one
1-(cis-3-methoxycyclobutyl)-3-methyl-8-(6-((2-(piperidin-1-yl)ethoxy)methyl)pyridin-3-yl)-1H-imidazo[4,5-c]cinnolin-2(3H)-one
8-(6-((2-(4-fluoropiperidin-1-yl)ethoxy)methyl)pyridin-3-yl)-1-(cis-3-methoxycyclobutyl)-3-methyl-1H-imidazo[4,5-c]cinnolin-2(3H)-one
8-(6-((2-(3-azabicyclo[3.1.0]hexan-3-yl)ethoxy)methyl)pyridin-3-yl)-1-(cis-3-methoxycyclobutyl)-3-methyl-1H-imidazo[4,5-c]cinnolin-2(3H)-one
8-(6-((2-(dimethylamino)ethoxy)methyl)pyridin-3-yl)-1-(cis-3-methoxycyclobutyl)-3-methyl-1H-imidazo[4,5-c]cinnolin-2(3H)-one
8-(6-((2-(dimethylamino)ethoxy)methyl)pyridin-3-yl)-3-methyl-1-(oxetan-3-yl)-1H-imidazo[4,5-c]cinnolin-2(3H)-one
3-methyl-1-(oxetan-3-yl)-8-(6-((2-(piperidin-1-yl)ethoxy)methyl)pyridin-3-yl)-1H-imidazo[4,5-c]cinnolin-2(3H)-one
8-(6-((2-(4-fluoropiperidin-1-yl)ethoxy)methyl)pyridin-3-yl)-3-methyl-1-(oxetan-3-yl)-1H-imidazo[4,5-c]cinnolin-2(3H)-one
8-(6-((2-(4-fluoropiperidin-1-yl)ethoxy)methyl)pyridin-3-yl)-1-((1s,3s)-3-methoxycyclobutyl)-3-methyl-1H-imidazo[4,5-c]cinnolin-2(3H)-one
3-methyl-1-(oxetan-3-yl)-8-(6-((2-(pyrrolidin-1-yl)ethoxy) methyl)pyridin-3-yl)-1H-imidazo[4,5-c]cinnolin-2(3H)-one
(S)-8-(6-((2-(4-fluoropiperidin-1-yl)ethoxy)methyl)pyridin-3-yl)-3-methyl-1-(tetrahydro-2H-pyran-3-yl)-1H-imidazo[4,5-c]cinnolin-2(3H)-one
8-(6-((2-(3-azabicyclo[3.1.0]hexan-3-yl)ethoxy)methyl)pyridin-3-yl)-3-methyl-1-((S)-tetrahydro-2H-pyran-3-yl)-1H-imidazo[4,5-c]cinnolin-2(3H)-one
(S)-1-isopropyl-3-methyl-8-(6-(2,2,2-trifluoro-1-(2-(pyrrolidin-1-yl)ethoxy)ethyl)pyridin-3-yl)-1,3-dihydro-2H-imidazo[4,5-c]cinnolin-2-one
(R)-1-isopropyl-3-methyl-8-(6-(2,2,2-trifluoro-1-(2-(pyrrolidin-1-yl)ethoxy)ethyl)pyridin-3-yl)-1,3-dihydro-2H-imidazo[4,5-c]cinnolin-2-one
1-isopropyl-3-methyl-8-(6-(2,2,2-trifluoro-1-(2-(pyrrolidin-1-yl)ethoxy)ethyl)pyridin-3-yl)-1,3-dihydro-2H-imidazo[4,5-c]cinnolin-2-one
1-isopropyl-3-methyl-8-(6-(2,2,2-trifluoro-1-(2-(3-methylpyrrolidin-1-yl)ethoxy)ethyl)pyridin-3-yl)-1,3-dihydro-2H-imidazo[4,5-c]cinnolin-2-one
1-isopropyl-3-methyl-8-(6-(2,2,2-trifluoro-1-(2-(3-methoxypyrrolidin-1-yl)ethoxy)ethyl)pyridin-3-yl)-1,3-dihydro-2H-imidazo[4,5-c]cinnolin-2-one
8-(6-(1-(2-(3,3-dimethylpyrrolidin-1-yl)ethoxy)-2,2,2-trifluoroethyl)pyridin-3-yl)-1-isopropyl-3-methyl-1,3-dihydro-2H-imidazo[4,5-c]cinnolin-2-one
8-(6-(1-(2-(5-azaspiro[2.4]heptan-5-yl)ethoxy)-2,2,2-trifluoroethyl)pyridin-3-yl)-1-isopropyl-3-methyl-1,3-dihydro-2H-imidazo[4,5-c]cinnolin-2-one
8-(6-(1-(2-(3-azabicyclo[3.1.0]hexan-3-yl)ethoxy)-2,2,2-trifluoroethyl)pyridin-3-yl)-1-isopropyl-3-methyl-1,3-dihydro-2H-imidazo[4,5-c]cinnolin-2-one
1-isopropyl-3-methyl-8-(6-(2,2,2-trifluoro-1-(2-(piperidin-1-yl)ethoxy)ethyl)pyridin-3-yl)-1,3-dihydro-2H-imidazo[4,5-c]cinnolin-2-one
8-(6-(1-(2-(4,4-dimethylpiperidin-1-yl)ethoxy)-2,2,2-trifluoroethyl)pyridin-3-yl)-1-isopropyl-3-methyl-1,3-dihydro-2H-imidazo[4,5-c]cinnolin-2-one
1-isopropyl-3-methyl-8-(6-(2,2,2-trifluoro-1-(2-(4-methoxypiperidin-1-yl)ethoxy)ethyl)pyridin-3-yl)-1,3-dihydro-2H-imidazo[4,5-c]cinnolin-2-one
8-(6-(1-(2-(6-azaspiro[2.5]octan-6-yl)ethoxy)-2,2,2-trifluoroethyl)pyridin-3-yl)-1-isopropyl-3-methyl-1,3-dihydro-2H-imidazo[4,5-c]cinnolin-2-one
3-methyl-1-(tetrahydro-2H-pyran-4-yl)-8-(6-(2,2,2-trifluoro-1-(2-(pyrrolidin-1-yl)ethoxy)ethyl)pyridin-3-yl)-1,3-dihydro-2H-imidazo[4,5-c]cinnolin-2-one
3-methyl-1-(tetrahydro-2H-pyran-4-yl)-8-(6-(2,2,2-trifluoro-1-(2-(3-methylpyrrolidin-1-yl)ethoxy)ethyl)pyridin-3-yl)-1,3-dihydro-2H-imidazo[4,5-c]cinnolin-2-one
3-methyl-1-(tetrahydro-2H-pyran-4-yl)-8-(6-(2,2,2-trifluoro-1-(2-(3-methoxypyrrolidin-1-yl)ethoxy)ethyl)pyridin-3-yl)-1,3-dihydro-2H-imidazo[4,5-c]cinnolin-2-one
8-(6-(1-(2-(3,3-dimethylpyrrolidin-1-yl)ethoxy)-2,2,2-trifluoroethyl)pyridin-3-yl)-3-methyl-1-(tetrahydro-2H-pyran-4-yl)-1,3-dihydro-2H-imidazo[4,5-c]cinnolin-2-one
8-(6-(1-(2-(5-azaspiro[2.4]heptan-5-yl)ethoxy)-2,2,2-trifluoroethyl)pyridin-3-yl)-3-methyl-1-(tetrahydro-2H-pyran-4-yl)-1,3-dihydro-2H-imidazo[4,5-c]cinnolin-2-one
8-(6-(1-(2-(3-azabicyclo[3.1.0]hexan-3-yl)ethoxy)-2,2,2-trifluoroethyl)pyridin-3-yl)-3-methyl-1-(tetrahydro-2H-pyran-4-yl)-1,3-dihydro-2H-imidazo[4,5-c]cinnolin-2-one
3-methyl-1-(tetrahydro-2H-pyran-4-yl)-8-(6-(2,2,2-trifluoro-1-(2-(piperidin-1-yl)ethoxy)ethyl)pyridin-3-yl)-1,3-dihydro-2H-imidazo[4,5-c]cinnolin-2-one
8-(6-(1-(2-(4,4-dimethylpiperidin-1-yl)ethoxy)-2,2,2-trifluoroethyl)pyridin-3-yl)-3-methyl-1-(tetrahydro-2H-pyran-4-yl)-1,3-dihydro-2H-imidazo[4,5-c]cinnolin-2-one
3-methyl-1-(tetrahydro-2H-pyran-4-yl)-8-(6-(2,2,2-trifluoro-1-(2-(4-methoxypiperidin-1-yl)ethoxy)ethyl)pyridin-3-yl)-1,3-dihydro-2H-imidazo[4,5-c]cinnolin-2-one
8-(6-(1-(2-(6-azaspiro[2.5]octan-6-yl)ethoxy)-2,2,2-trifluoroethyl)pyridin-3-yl)-3-methyl-1-(tetrahydro-2H-pyran-4-yl)-1,3-dihydro-2H-imidazo[4,5-c]cinnolin-2-one
1-(3-methoxycyclobutyl)-3-methyl-8-(6-(2,2,2-trifluoro-1-(2-(pyrrolidin-1-yl)ethoxy)ethyl)pyridin-3-yl)-1,3-dihydro-2H-imidazo[4,5-c]cinnolin-2-one
1-(3-methoxycyclobutyl)-3-methyl-8-(6-(2,2,2-trifluoro-1-(2-(3-methylpyrrolidin-1-yl)ethoxy)ethyl)pyridin-3-yl)-1,3-dihydro-2H-imidazo[4,5-c]cinnolin-2-one
1-(3-methoxycyclobutyl)-3-methyl-8-(6-(2,2,2-trifluoro-1-(2-(3-methoxypyrrolidin-1-yl)ethoxy)ethyl)pyridin-3-yl)-1,3-dihydro-2H-imidazo[4,5-c]cinnolin-2-one
8-(6-(1-(2-(3,3-dimethylpyrrolidin-1-yl)ethoxy)-2,2,2-trifluoroethyl)pyridin-3-yl)-1-(3-methoxycyclobutyl)-3-methyl-1,3-dihydro-2H-imidazo[4,5-c]cinnolin-2-one
8-(6-(1-(2-(5-azaspiro[2.4]heptan-5-yl)ethoxy)-2,2,2-trifluoroethyl)pyridin-3-yl)-1-(3-methoxycyclobutyl)-3-methyl-1,3-dihydro-2H-imidazo[4,5-c]cinnolin-2-one
8-(6-(1-(2-(3-azabicyclo[3.1.0]hexan-3-yl)ethoxy)-2,2,2-trifluoroethyl)pyridin-3-yl)-1-(3-methoxycyclobutyl)-3-methyl-1,3-dihydro-2H-imidazo[4,5-c]cinnolin-2-one
1-(3-methoxycyclobutyl)-3-methyl-8-(6-(2,2,2-trifluoro-1-(2-(piperidin-1-yl)ethoxy)ethyl)pyridin-3-yl)-1,3-dihydro-2H-imidazo[4,5-c]cinnolin-2-one
8-(6-(1-(2-(4,4-dimethylpiperidin-1-yl)ethoxy)-2,2,2-trifluoroethyl)pyridin-3-yl)-1-(3-methoxycyclobutyl)-3-methyl-1,3-dihydro-2H-imidazo[4,5-c]cinnolin-2-one
1-(3-methoxycyclobutyl)-3-methyl-8-(6-(2,2,2-trifluoro-1-(2-(4-methoxypiperidin-1-yl)ethoxy)ethyl)pyridin-3-yl)-1,3-dihydro-2H-imidazo[4,5-c]cinnolin-2-one
8-(6-(1-(2-(6-azaspiro[2.5]octan-6-yl)ethoxy)-2,2,2-trifluoroethyl)pyridin-3-yl)-1-(3-methoxycyclobutyl)-3-methyl-1,3-dihydro-2H-imidazo[4,5-c]cinnolin-2-one
(R)-2-(1-(5-(1-isopropyl-3-methyl-2-oxo-2,3-dihydro-1H-imidazo[4,5-c]cinnolin-8-yl)pyridin-2-yl)ethoxy)acetic acid
8-(6-(2-(2-(3-azabicyclo[3.1.0]hexan-3-yl)ethoxy)propan-2-yl)pyridin-3-yl)-1-isopropyl-3-methyl-1H-imidazo[4,5-c]cinnolin-2(3H)-one
8-(6-((2-(3-azabicyclo[3.1.0]hexan-3-yl)-1,1-difluoroethoxy)methyl)pyridin-3-yl)-1-isopropyl-3-methyl-1H-imidazo[4,5-c]cinnolin-2(3H)-one
1-isopropyl-3-methyl-8-(6-(1-(2-(pyrrolidin-1-yl)ethoxy)propyl)pyridin-3-yl)-1H-imidazo[4,5-c]cinnolin-2(3H)-one
1-isopropyl-3-methyl-8-(6-(1-(2-(pyrrolidin-1-yl)ethoxy)butyl)pyridin-3-yl)-1H-imidazo[4,5-c]cinnolin-2(3H)-one
1-isopropyl-3-methyl-8-(6-(2-methyl-1-(2-(pyrrolidin-1-yl)ethoxy)propyl)pyridin-3-yl)-1H-imidazo[4,5-c]cinnolin-2(3H)-one, and
1-isopropyl-3-methyl-8-(6-(4-methyl-1-(2-(pyrrolidin-1-yl)ethoxy)pentyl)pyridin-3-yl)-1H-imidazo[4,5-c]cinnolin-2(3H)-one.

Exemplary compounds of Formula (I) are set forth in Table 1 below.

TABLE 1

| Cmpd | |
|---|---|
| No. | Compound Structure and Name |
| 1 | 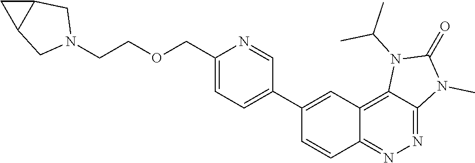 [SMILES: CC(C)n1c(=O)n(C)c2nnc3ccc(-c4ccc(COCCN5CC6CC6C5)nc4)cc3c21] |
| | 8-(6-((2-(3-azabicyclo[3.1.0]hexan-3-yl)ethoxy)methyl)pyridin-3-yl)-1- |
| | isopropyl-3-methyl-1H-imidazo[4,5-c]cinnolin-2(3H)-one |
| 2 | 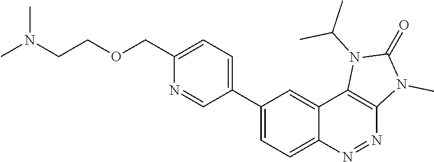 |
| | 8-(6-((2-(dimethylamino)ethoxy)methyl)pyridin-3-yl)-1-isopropyl- |
| | 3-methyl-1H-imidazo[4,5-c]cinnolin-2(3H)-one |
| 3 | 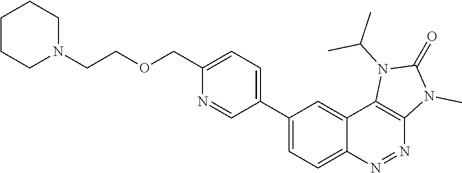 [SMILES: CC(C)n1c(=O)n(C)c2nnc3ccc(-c4ccc(COCCN5CCCCC5)nc4)cc3c21] |
| | 1-isopropyl-3-methyl-8-(6-((2-(piperidin-1-yl)ethoxy)methyl)pyridin-3- |
| | yl)-1H-imidazo[4,5-c]cinnolin-2(3H)-one |
| 4 | 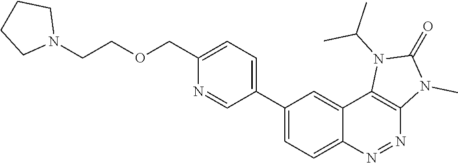 [SMILES: CC(C)n1c(=O)n(C)c2nnc3ccc(-c4ccc(COCCN5CCCC5)nc4)cc3c21] |
| | 1-isopropyl-3-methyl-8-(6-((2-(pyrrolidin-1-yl)ethoxy)methyl)pyridin- |
| | 3-yl)-1H-imidazo[4,5-c]cinnolin-2(3H)-one |
| 5 | 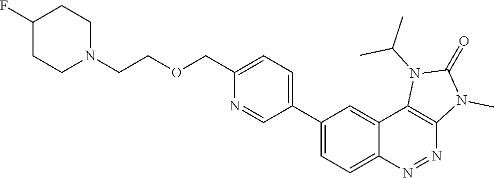 [SMILES: CC(C)n1c(=O)n(C)c2nnc3ccc(-c4ccc(COCCN5CCC(F)CC5)nc4)cc3c21] |
| | 8-(6-((2-(4-fluoropiperidin-1-yl)ethoxy)methyl)pyridin-3-yl)-1-isopropyl- |
| | 3-methyl-1H-imidazo[4,5-c]cinnolin-2(3H)-one |
| 6 | 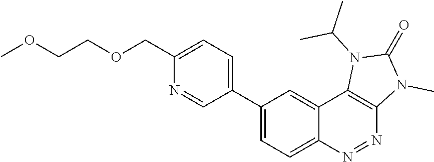 |
| | 1-isopropyl-8-(6-((2-methoxyethoxy)methyl)pyridin-3-yl)-3-methyl- |
| | 1H-imidazo[4,5-c]cinnolin-2(3H)-one |
| 7 | 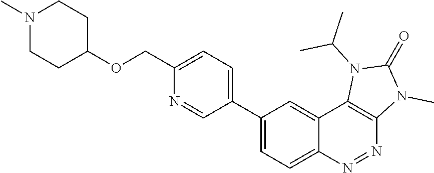 |
| | 1-isopropyl-3-methyl-8-(6-(((1-methylpiperidin-4-yl)oxy)methyl)- |
| | pyridin-3-yl)-1H-imidazo[4,5-c]cinnolin-2(3H)-one |
| 8 | 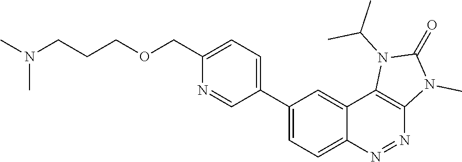 |
| | 8-(6-((3-(dimethylamino)propoxy)methyl)pyridin-3-yl)-1-isopropyl-3- |
| | methyl-1H-imidazo[4,5-c]cinnolin-2(3H)-one |
| 9 | 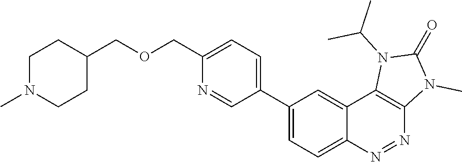 |
| | 1-isopropyl-3-methyl-8-(6-(((1-methylpiperidin-4-yl)methoxy)methyl)- |
| | pyridin-3-yl)-1H-imidazo[4,5-c]cinnolin-2(3H)-one |
| 10 | 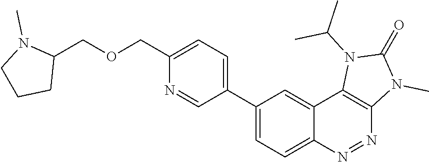 [SMILES: CC(C)n1c(=O)n(C)c2nnc3ccc(-c4ccc(COCC5CCCN5C)nc4)cc3c21] |
| | 1-isopropyl-3-methyl-8-(6-(((1-methylpyrrolidin-2-yl)methoxy)- |
| | methyl)pyridin-3-yl)-1H-imidazo[4,5-c]cinnolin-2(3H)-one |
| 11 | 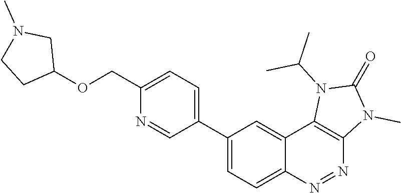 [SMILES: CC(C)n1c(=O)n(C)c2nnc3ccc(-c4ccc(COC5CCN(C)C5)nc4)cc3c21] |
| | 1-isopropyl-3-methyl-8-(6-(((1-methylpyrrolidin-3-yl)oxy)- |
| | methyl)pyridin-3-yl)-1H-imidazo[4,5-c]cinnolin-2(3H)-one |
| 12 | 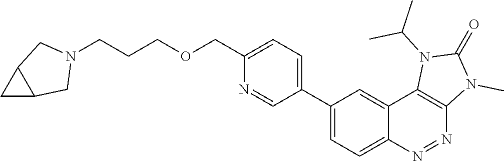 [SMILES: CC(C)n1c(=O)n(C)c2nnc3ccc(-c4ccc(COCCCN5CC6CC6C5)nc4)cc3c21] |
| | 8-(6-((3-(3-azabicyclo[3.1.0]hexan-3-yl)propoxy)methyl)pyridin-3-yl)- |
| | 1-isopropyl-3-methyl-1H-imidazo[4,5-c]cinnolin-2(3H)-one |
| 13 | 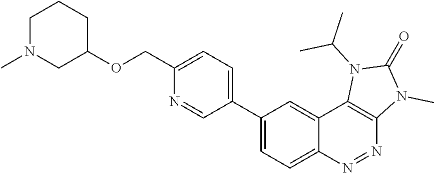 |
| | 1-isopropyl-3-methyl-8-(6-(((1-methylpiperidin-3-yl)oxy)methyl)- |
| | pyridin-3-yl)-1H-imidazo[4,5-c]cinnolin-2(3H)-one |
| 14 | 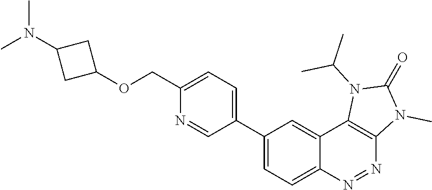 |
| | 8-(6-((3-(dimethylamino)cyclobutoxy)methyl)pyridin-3-yl)-1- |
| | isopropyl-3-methyl-1H-imidazo[4,5-c]cinnolin-2(3H)-one |
| 15 | 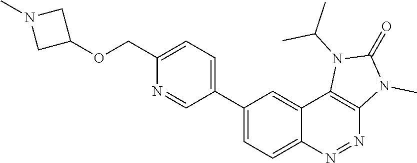 |
| | 1-isopropyl-3-methyl-8-(6-(((1-methylazetidin-3-yl)oxy)methyl)- |
| | pyridin-3-yl)-1H-imidazo[4,5-c]cinnolin-2(3H)-one |
| 16 | 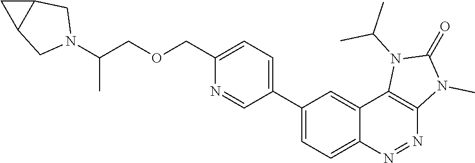 [SMILES: CC(COCc1ccc(-c2ccc3nnc4c(c3c2)n(C(C)C)c(=O)n4C)cn1)N1CC2CC2C1] |
| | 8-(6-((2-(3-azabicyclo[3.1.0]hexan-3-yl)propoxy)methyl)pyridin-3-yl)- |
| | 1-isopropyl-3-methyl-1H-imidazo[4,5-c]cinnolin-2(3H)-one |
| 17 | 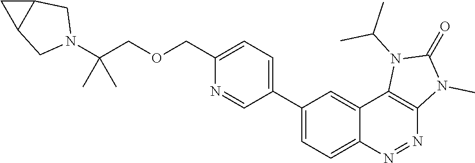 [SMILES: CC(C)n1c(=O)n(C)c2nnc3ccc(-c4ccc(COCC(C)(C)N5CC6CC6C5)nc4)cc3c21] |
| | 8-(6-((2-(3-azabicyclo[3.1.0]hexan-3-yl)-2-methylpropoxy)methyl)pyridin- |
| | 3-yl)-1-isopropyl-3-methyl-1H-imidazo[4,5-c]cinnolin-2(3H)-one |
| 18 | 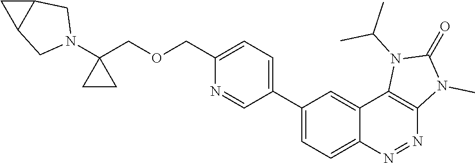 [SMILES: CC(C)n1c(=O)n(C)c2nnc3ccc(-c4ccc(COCC5(N6CC7CC7C6)CC5)nc4)cc3c21] |
| | 8-(6-(((1-(3-azabicyclo[3.1.0]hexan-3-yl)cyclopropyl)methoxy)methyl)- |
| | pyridin-3-yl)-1-isopropyl-3-methyl-1H-imidazo[4,5-c]cinnolin- |
| | 2(3H)-one |
| 19 | 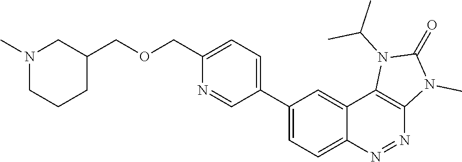 |
| | 1-isopropyl-3-methyl-8-(6-(((1-methylpiperidin-3-yl)methoxy)methyl)- |
| | pyridin-3-yl)-1H-imidazo[4,5-c]cinnolin-2(3H)-one |
| 20 | 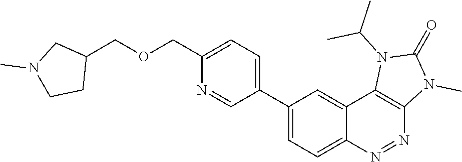 |
| | 1-isopropyl-3-methyl-8-(6-(((1-methylpyrrolidin-3-yl)methoxy)- |
| | methyl)pyridin-3-yl)-1H-imidazo[4,5-c]cinnolin-2(3H)-one |
| 21 | 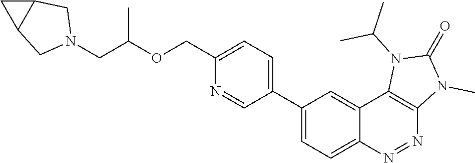 [SMILES: CC(CN1CC2CC2C1)OCc1ccc(-c2ccc3nnc4c(c3c2)n(C(C)C)c(=O)n4C)cn1] |
| | 8-(6-(((1-(3-azabicyclo[3.1.0]hexan-3-yl)propan-2-yl)oxy)methyl)pyridin- |
| | 3-yl)-1-isopropyl-3-methyl-1H-imidazo[4,5-c]cinnolin-2(3H)-one |
| 22 | 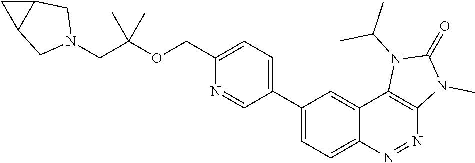 [SMILES: CC(C)n1c(=O)n(C)c2nnc3ccc(-c4ccc(COC(C)(C)CN5CC6CC6C5)nc4)cc3c21] |
| | 8-(6-(((1-(3-azabicyclo[3.1.0]hexan-3-yl)-2-methylpropan-2-yl)oxy)- |
| | methyl)pyridin-3-yl)-1-isopropyl-3-methyl-1H-imidazo[4,5-c]- |
| | cinnolin-2(3H)-one |
| 23 | 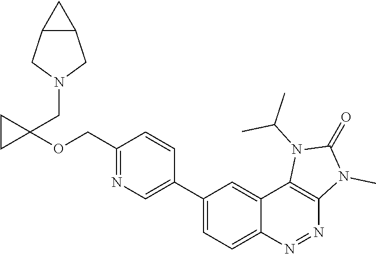 [SMILES: CC(C)n1c(=O)n(C)c2nnc3ccc(-c4ccc(COC5(CN6CC7CC7C6)CC5)nc4)cc3c21] |
| | 8-(6-((1-(3-azabicyclo[3.1.0]hexan-3-ylmethyl)cyclo- |
| | propoxy)methyl)pyridin-3-yl)-1-isopropyl-3-methyl- |
| | 1H-imidazo[4,5-c]cinnolin-2(3H)-one |
| 24 | 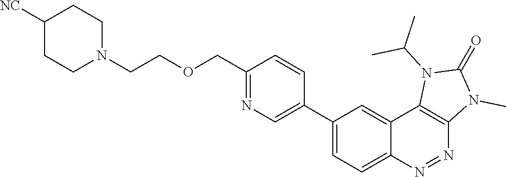 [SMILES: CC(C)n1c(=O)n(C)c2nnc3ccc(-c4ccc(COCCN5CCC(C#N)CC5)nc4)cc3c21] |
| | 1-(2-((5-(1-isopropyl-3-methyl-2-oxo-2,3-dihydro-1H-imidazo[4,5-c]- |
| | cinnolin-8-yl)pyridin-2-yl)methoxy)ethyl)piperidine-4-carbonitrile |
| 25 | 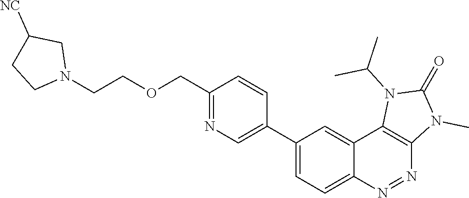 [SMILES: CC(C)n1c(=O)n(C)c2nnc3ccc(-c4ccc(COCCN5CCC(C#N)C5)nc4)cc3c21] |
| | 1-(2-((R)-1-(5-(1-isopropyl-3-methyl-2-oxo-2,3-dihydro-1H-imidazo[4,5-c]- |
| | cinnolin-8-yl)pyridin-2-yl)ethoxy)ethyl)-3-methylpyrrolidine-3-carbonitrile |
| 26 | 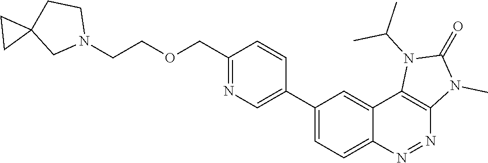 [SMILES: CC(C)n1c(=O)n(C)c2nnc3ccc(-c4ccc(COCCN5CCC6(CC6)C5)nc4)cc3c21] |
| | 8-(6-((2-(5-azaspiro[2.4]heptan-5-yl)ethoxy)methyl)pyridin-3-yl)-1-isopropyl- |
| | 3-methyl-1H-imidazo[4,5-c]cinnolin-2(3H)-one |
| 27 | 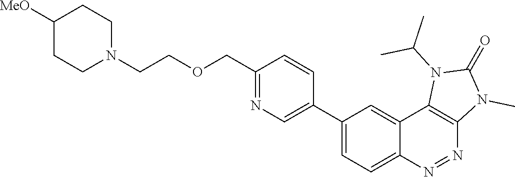 [SMILES: COC1CCN(CCOCc2ccc(-c3ccc4nnc5c(c4c3)n(C(C)C)c(=O)n5C)cn2)CC1] |
| | 1-isopropyl-8-(6-((2-(4-methoxypiperidin-1-yl)ethoxy)methyl) pyridin-3- |
| | yl)-3-methyl-1H-imidazo[4,5-c]cinnolin-2(3H)-one |
| 28 | 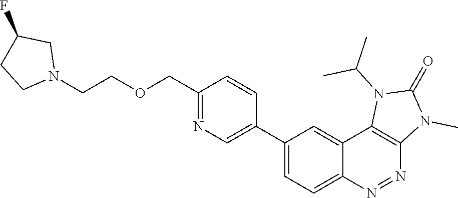 [SMILES: CC(C)n1c(=O)n(C)c2nnc3ccc(-c4ccc(COCCN5CC[C@@H](F)C5)nc4)cc3c21] |
| | (R)-8-(6-((2-(3-fluoropyrrolidin-1-yl)ethoxy)methyl)pyridin-3-yl)-1- |
| | isopropyl-3-methyl-1H-imidazo[4,5-c]cinnolin-2(3H)-one |
| 29 | 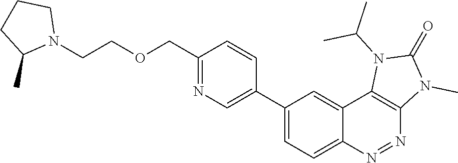 [SMILES: CC(C)n1c(=O)n(C)c2nnc3ccc(-c4ccc(COCCN5CCC[C@@H]5C)nc4)cc3c21] |
| | (S)-1-isopropyl-3-methyl-8-(6-((2-(2-methylpyrrolidin-1-yl)ethoxy)- |
| | methyl)pyridin-3-yl)-1H-imidazo[4,5-c]cinnolin-2(3H)-one |
| 30 | 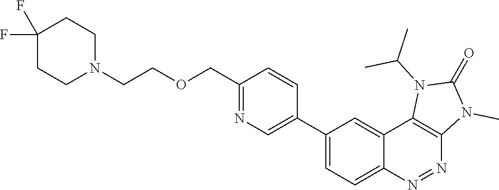 [SMILES: CC(C)n1c(=O)n(C)c2nnc3ccc(-c4ccc(COCCN5CCC(F)(F)CC5)nc4)cc3c21] |
| | 8-(6-((2-(4,4-difluoropiperidin-1-yl)ethoxy)methyl)pyridin-3-yl)-1-isopropyl- |
| | 3-methyl-1H-imidazo[4,5-c]cinnolin-2(3H)-one |
| 31 | 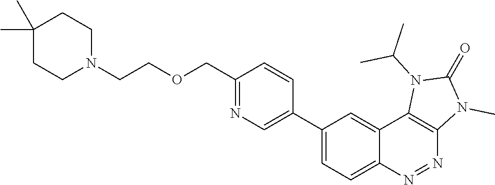 |
| | 8-(6-((2-(4,4-dimethylpiperidin-1-yl)ethoxy)methyl)pyridin-3-yl)-1-isopropyl- |
| | 3-methyl-1H-imidazo[4,5-c]cinnolin-2(3H)-one |
| 32 | 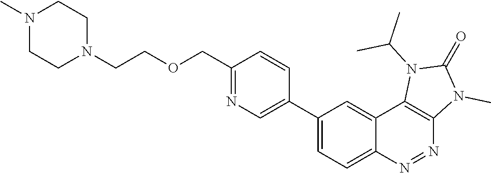 |
| | 1-isopropyl-3-methyl-8-(6-((2-(4-methylpiperazin-1-yl)ethoxy)methyl)pyridin- |
| | 3-yl)-1H-imidazo[4,5-c]cinnolin-2(3H)-one |
| 33 | 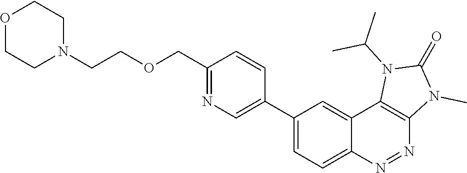 [SMILES: CC(C)n1c(=O)n(C)c2nnc3ccc(-c4ccc(COCCN5CCOCC5)nc4)cc3c21] |
| | 1-isopropyl-3-methyl-8-(6-((2-morpholinoethoxy)methyl) pyridin-3-yl)-1H- |
| | imidazo[4,5-c]cinnolin-2(3H)-one |
| 34 | 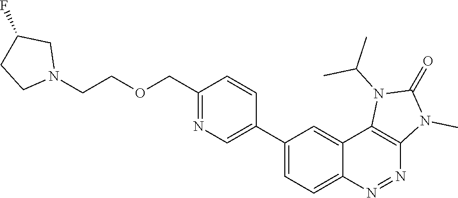 [SMILES: CC(C)n1c(=O)n(C)c2nnc3ccc(-c4ccc(COCCN5CC[C@H](F)C5)nc4)cc3c21] |
| | (S)-8-(6-((2-(3-fluoropyrrolidin-1-yl)ethoxy)methyl)pyridin-3-yl)-1- |
| | isopropyl-3-methyl-1H-imidazo[4,5-c]cinnolin-2(3H)-one |
| 35 | 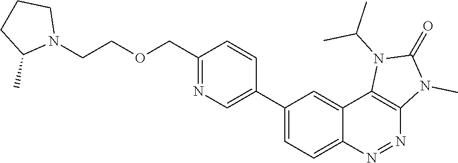 [SMILES: CC(C)n1c(=O)n(C)c2nnc3ccc(-c4ccc(COCCN5CCC[C@H]5C)nc4)cc3c21] |
| | (R)-1-isopropyl-3-methyl-8-(6-((2-(2-methylpyrrolidin-1-yl)ethoxy)- |
| | methyl)pyridin-3-yl)-1H-imidazo[4,5-c]cinnolin-2(3H)-one |
| 36 | 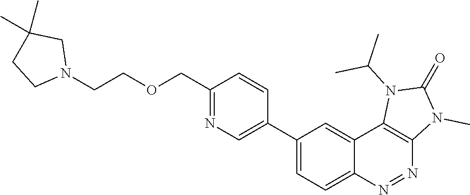 |
| | 8-(6-((2-(3,3-dimethylpyrrolidin-1-yl)ethoxy)methyl)pyridin-3-yl)-1- |
| | isopropyl-3-methyl-1H-imidazo[4,5-c]cinnolin-2(3H)-one |
| 37 | 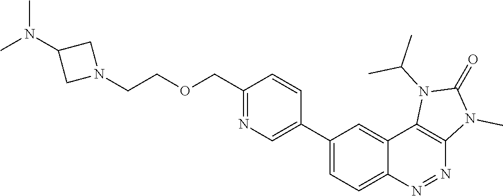 |
| | 8-(6-((2-(3-(dimethylamino)azetidin-1-yl)ethoxy)methyl)pyridin-3-yl)-1- |
| | isopropyl-3-methyl-1H-imidazo[4,5-c]cinnolin-2(3H)-one |
| 38 | 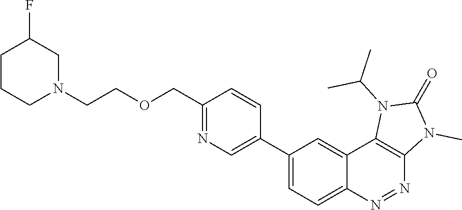 [SMILES: CC(C)n1c(=O)n(C)c2nnc3ccc(-c4ccc(COCCN5CCCC(F)C5)nc4)cc3c21] |
| | 8-(6-((2-(3-fluoropiperidin-1-yl)ethoxy)methyl)pyridin-3-yl)-1- |
| | isopropyl-3-methyl-1H-imidazo[4,5-c]cinnolin-2(3H)-one |
| 39 | 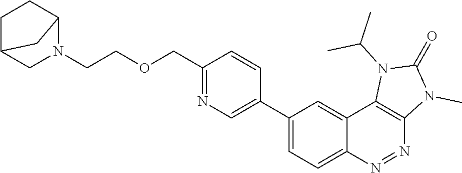 [SMILES: CC(C)n1c(=O)n(C)c2nnc3ccc(-c4ccc(COCCN5CC6CCC5C6)nc4)cc3c21] |
| | 8-(6-((2-(2-azabicyclo[2.2.1]heptan-2-yl)ethoxy)methyl)pyridin-3-yl)-1- |
| | isopropyl-3-methyl-1H-imidazo[4,5-c]cinnolin-2(3H)-one |
| 40 | 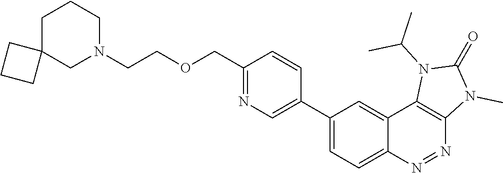 [SMILES: CC(C)n1c(=O)n(C)c2nnc3ccc(-c4ccc(COCCN5CCCC6(CCC6)C5)nc4)cc3c21] |
| | 8-(6-((2-(6-azaspiro[3.5]nonan-6-yl)ethoxy)methyl)pyridin-3-yl)-1- |
| | isopropyl-3-methyl-1H-imidazo[4,5-c]cinnolin-2(3H)-one |
| 41 | 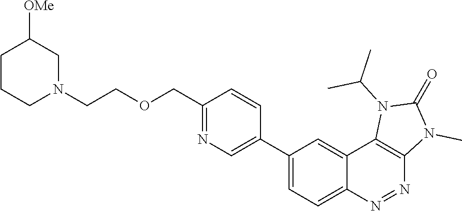 [SMILES: COC1CCCN(CCOCc2ccc(-c3ccc4nnc5c(c4c3)n(C(C)C)c(=O)n5C)cn2)C1] |
| | 1-isopropyl-8-(6-((2-(3-methoxypiperidin-1-yl)ethoxy)methyl)pyridin- |
| | 3-yl)-3-methyl-1H-imidazo[4,5-c]cinnolin-2(3H)-one |
| 42 | 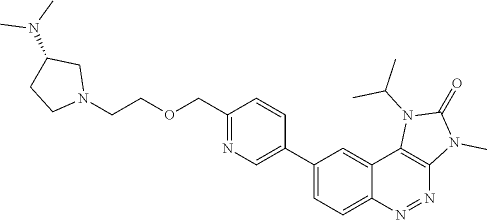 |
| | (S)-8-(6-((2-(3-(dimethylamino)pyrrolidin-1-yl)ethoxy)methyl) pyridin-3- |
| | yl)-1-isopropyl-3-methyl-1H-imidazo[4,5-c]cinnolin-2(3H)-one |
| 43 | 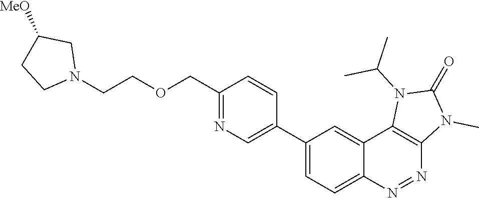 [SMILES: CO[C@H]1CCN(CCOCc2ccc(-c3ccc4nnc5c(c4c3)n(C(C)C)c(=O)n5C)cn2)C1] |
| | (S)-1-isopropyl-8-(6-((2-(3-methoxypyrrolidin-1-yl)ethoxy)methyl)pyridin- |
| | 3-yl)-3-methyl-1H-imidazo[4,5-c]cinnolin-2(3H)-one |
| 44 | 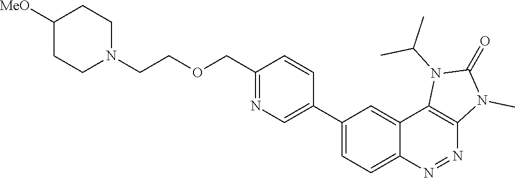 [SMILES: COC1CCN(CCOCc2ccc(-c3ccc4nnc5c(c4c3)n(C(C)C)c(=O)n5C)cn2)CC1] |
| | 1-isopropyl-8-(6-((2-(4-methoxypiperidin-1-yl)ethoxy)methyl)pyridin-3-yl)- |
| | 3-methyl-1H-imidazo[4,5-c]cinnolin-2(3H)-one |
| 45 | 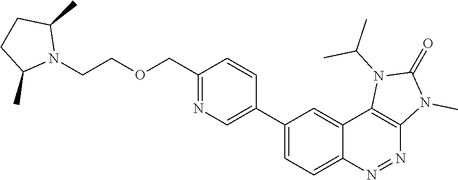 [SMILES: CC(C)n1c(=O)n(C)c2nnc3ccc(-c4ccc(COCCN5[C@H](C)CC[C@@H]5C)nc4)cc3c21] |
| | 8-(6-((2-((2R,5S)-2,5-dimethylpyrrolidin-1-yl)ethoxy)methyl)pyridin-3- |
| | yl)-1-isopropyl-3-methyl-1H-imidazo[4,5-c]cinnolin-2(3H)-one |
| 46 | 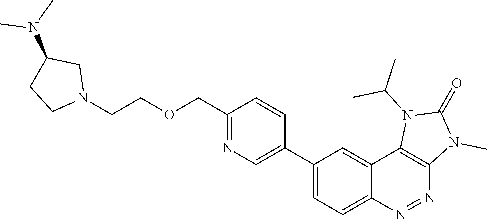 |
| | (R)-8-(6-((2-(3-(dimethylamino)pyrrolidin-1-yl)ethoxy)methyl)pyridin-3- |
| | yl)-1-isopropyl-3-methyl-1H-imidazo[4,5-c]cinnolin-2(3H)-one |
| 47 | 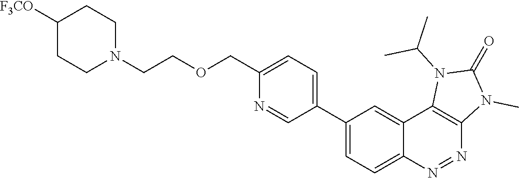 [SMILES: CC(C)n1c(=O)n(C)c2nnc3ccc(-c4ccc(COCCN5CCC(OC(F)(F)F)CC5)nc4)cc3c21] |
| | 1-isopropyl-3-methyl-8-(6-((2-(4-(trifluoromethoxy)piperidin-1-yl)ethoxy)- |
| | methyl)pyridin-3-yl)-1H-imidazo[4,5-c]cinnolin-2(3H)-one |
| 48 | 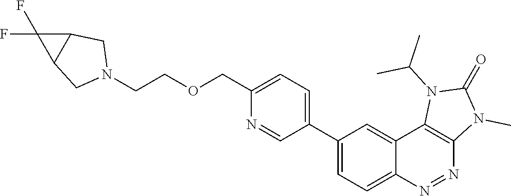 [SMILES: CC(C)n1c(=O)n(C)c2nnc3ccc(-c4ccc(COCCN5CC6C(C5)C6(F)F)nc4)cc3c21] |
| | 8-(6-((2-(6,6-difluoro-3-azabicyclo[3.1.0]hexan-3-yl)ethoxy)methyl)pyridin- |
| | 3-yl)-1-isopropyl-3-methyl-1H-imidazo[4,5-c]cinnolin-2(3H)-one |
| 49 | 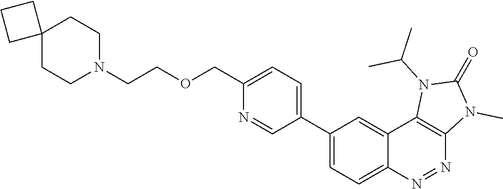 [SMILES: CC(C)n1c(=O)n(C)c2nnc3ccc(-c4ccc(COCCN5CCC6(CCC6)CC5)nc4)cc3c21] |
| | 8-(6-((2-(7-azaspiro[3.5]nonan-7-yl)ethoxy)methyl)pyridin-3-yl)-1-isopropyl- |
| | 3-methyl-1H-imidazo[4,5-c]cinnolin-2(3H)-one |
| 50 | 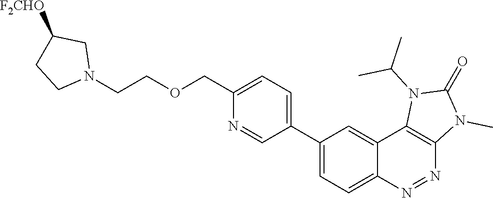 |
| | (R)-8-(6-((2-(3-(difluoromethoxy)pyrrolidin-1-yl)ethoxy)methyl)pyridin-3- |
| | yl)-1-isopropyl-3-methyl-1H-imidazo[4,5-c]cinnolin-2(3H)-one |
| 51 | 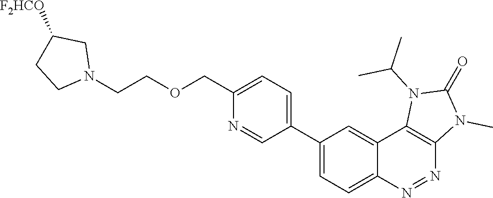 [SMILES: CC(C)n1c(=O)n(C)c2nnc3ccc(-c4ccc(COCCN5CC[C@H](OC(F)F)C5)nc4)cc3c21] |
| | (S)-8-(6-((2-(3-(difluoromethoxy)pyrrolidin-1-yl)ethoxy)methyl)pyridin-3- |
| | yl)-1-isopropyl-3-methyl-1H-imidazo[4,5-c]cinnolin-2(3H)-one |
| 52 | 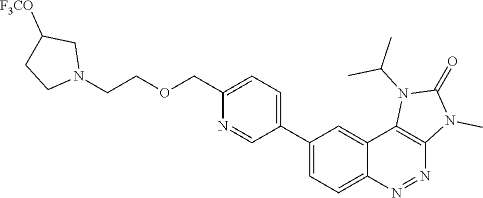 [SMILES: CC(C)n1c(=O)n(C)c2nnc3ccc(-c4ccc(COCCN5CCC(OC(F)(F)F)C5)nc4)cc3c21] |
| | 1-isopropyl-3-methyl-8-(6-((2-(3-(trifluoromethoxy)pyrrolidin-1-yl)ethoxy)- |
| | methyl)pyridin-3-yl)-1,3-dihydro-2H-imidazo[4,5-c]cinnolin-2-one |
| 53 | 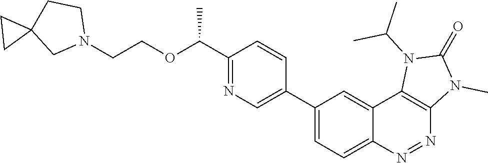 [SMILES: CC(C)n1c(=O)n(C)c2nnc3ccc(-c4ccc([C@@H](C)OCCN5CCC6(CC6)C5)nc4)cc3c21] |
| | (R)-8-(6-(1-(2-(5-azaspiro[2.4]heptan-5-yl)ethoxy)ethyl)pyridin-3-yl)-1- |
| | isopropyl-3-methyl-1H-imidazo[4,5-c]cinnolin-2(3H)-one |
| 54 | 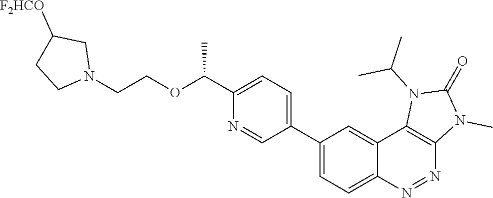 [SMILES: CC(C)n1c(=O)n(C)c2nnc3ccc(-c4ccc([C@@H](C)OCCN5CCC(OC(F)F)C5)nc4)cc3c21] |
| | (R)-8-(6-(1-(2-(6-azaspiro[2.5]octan-6-yl)ethoxy)ethyl)pyridin-3-yl)-1- |
| | isopropyl-3-methyl-1H-imidazo[4,5-c]cinnolin-2(3H)-one |
| 55 | 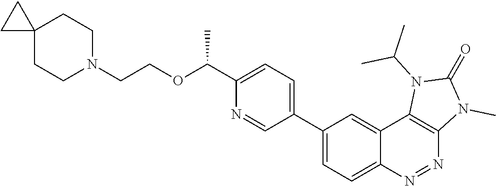 [SMILES: CC(C)n1c(=O)n(C)c2nnc3ccc(-c4ccc([C@@H](C)OCCN5CCC6(CC5)CC6)nc4)cc3c21] |
| | (R)-8-(6-(1-(2-(6-azaspiro[2.5]octan-6-yl)ethoxy)ethyl)pyridin-3-yl)-1- |
| | isopropyl-3-methyl-1H-imidazo[4,5-c]cinnolin-2(3H)-one |
| 56 | 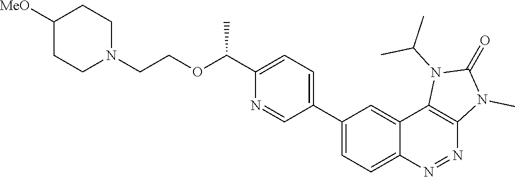 [SMILES: COC1CCN(CCO[C@H](C)c2ccc(-c3ccc4nnc5c(c4c3)n(C(C)C)c(=O)n5C)cn2)CC1] |
| | (R)-1-isopropyl-8-(6-(1-(2-(4-methoxypiperidin-1-yl)ethoxy)ethyl)pyridin- |
| | 3-yl)-3-methyl-1H-imidazo[4,5-c]cinnolin-2(3H)-one |
| 57 | 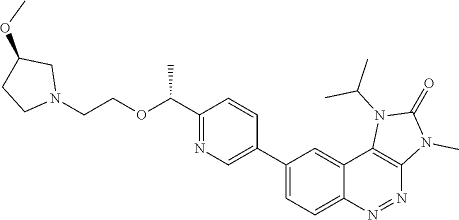 [SMILES: CO[C@@H]1CCN(CCO[C@H](C)c2ccc(-c3ccc4nnc5c(c4c3)n(C(C)C)c(=O)n5C)cn2)C1] |
| | 1-isopropyl-8-(6-((R)-1-(2-((R)-3-methoxypyrrolidin-1-yl)ethoxy)- |
| | ethyl)pyridin-3-yl)-3-methyl-1H-imidazo[4,5-c]cinnolin-2(3H)-one |
| 58 | 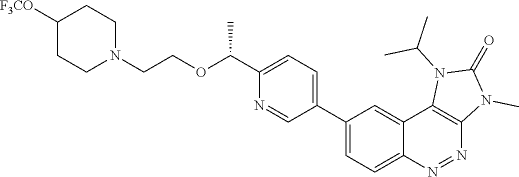 [SMILES: CC(C)n1c(=O)n(C)c2nnc3ccc(-c4ccc([C@@H](C)OCCN5CCC(OC(F)(F)F)CC5)nc4)cc3c21] |
| | (R)-1-isopropyl-3-methyl-8-(6-(1-(2-(4-(trifluoromethoxy)piperidin-1-yl)- |
| | ethoxy)ethyl)pyridin-3-yl)-1H-imidazo[4,5-c]cinnolin-2(3H)-one |
| 59 | 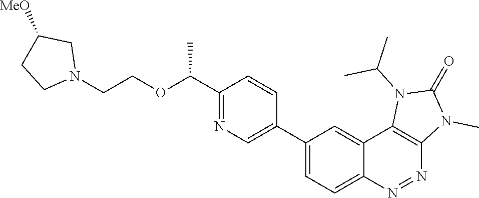 [SMILES: CO[C@H]1CCN(CCO[C@H](C)c2ccc(-c3ccc4nnc5c(c4c3)n(C(C)C)c(=O)n5C)cn2)C1] |
| | 1-isopropyl-8-(6-((R)-1-(2-((S)-3-methoxypyrrolidin-1-yl)ethoxy)ethyl)- |
| | pyridin-3-yl)-3-methyl-1H-imidazo[4,5-c]cinnolin-2(3H)-one |
| 60 | 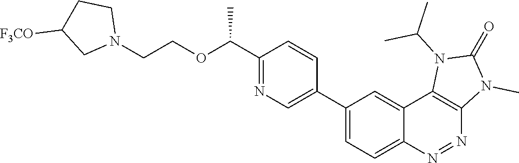 [SMILES: CC(C)n1c(=O)n(C)c2nnc3ccc(-c4ccc([C@@H](C)OCCN5CCC(OC(F)(F)F)C5)nc4)cc3c21] |
| | 8-(6-((2-(3,3-dimethylpyrrolidin-1-yl)ethoxy)methyl)pyridin-3-yl)-7-fluoro-1- |
| | isopropyl-3-methyl-1H-imidazo[4,5-c]cinnolin-2(3H)-one |
| 61 | 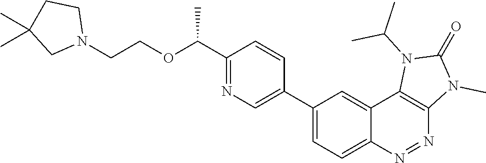 |
| | (R)-8-(6-(1-(2-(3,3-dimethylpyrrolidin-1-yl)ethoxy)ethyl)pyridin-3-yl)- |
| | 1-isopropyl-3-methyl-1H-imidazo[4,5-c]cinnolin-2(3H)-one |
| 62 | 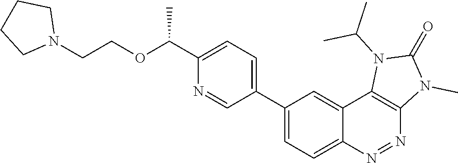 [SMILES: CC(C)n1c(=O)n(C)c2nnc3ccc(-c4ccc([C@@H](C)OCCN5CCCC5)nc4)cc3c21] |
| | (R)-1-isopropyl-3-methyl-8-(6-(1-(2-(pyrrolidin-1-yl)ethoxy)ethyl)- |
| | pyridin-3-yl)-1,3-dihydro-2H-imidazo[4,5-c]cinnolin-2-one |
| 63 | 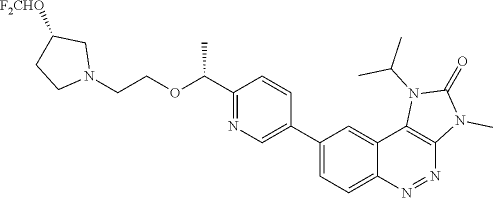 |
| | 8-(6-((R)-1-(2-((S)-3-(difluoromethoxy)pyrrolidin-1-yl)ethoxy)ethyl)pyridin- |
| | 3-yl)-1-isopropyl-3-methyl-1H-imidazo[4,5-c]cinnolin-2(3H)-one |
| 64 | 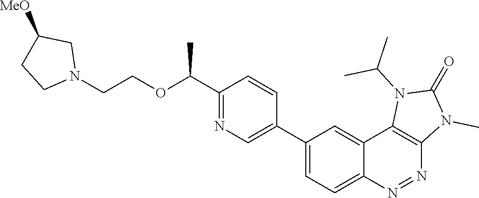 [SMILES: CO[C@@H]1CCN(CCO[C@@H](C)c2ccc(-c3ccc4nnc5c(c4c3)n(C(C)C)c(=O)n5C)cn2)C1] |
| | 1-isopropyl-8-(6-((S)-1-(2-((R)-3-methoxypyrrolidin-1-yl)ethoxy)ethyl)- |
| | pyridin-3-yl)-3-methyl-1H-imidazo[4,5-c]cinnolin-2(3H)-one |
| 65 | 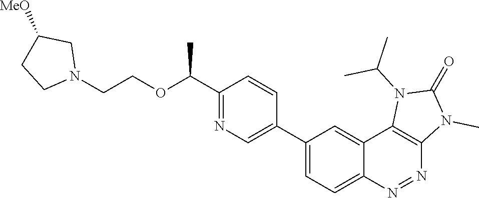 [SMILES: CO[C@H]1CCN(CCO[C@@H](C)c2ccc(-c3ccc4nnc5c(c4c3)n(C(C)C)c(=O)n5C)cn2)C1] |
| | 1-isopropyl-8-(6-((S)-1-(2-((S)-3-methoxypyrrolidin-1-yl)ethoxy)ethyl)- |
| | pyridin-3-yl)-3-methyl-1H-imidazo[4,5-c]cinnolin-2(3H)-one |
| 66 | 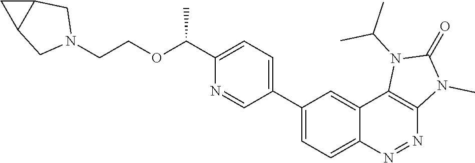 [SMILES: CC(C)n1c(=O)n(C)c2nnc3ccc(-c4ccc([C@@H](C)OCCN5CC6CC6C5)nc4)cc3c21] |
| | 8-(6-((1R)-1-(2-(3-azabicyclo[3.1.0]hexan-3-yl)ethoxy)ethyl)pyridin-3- |
| | yl)-1-isopropyl-3-methyl-1H-imidazo[4,5-c]cinnolin-2(3H)-one |
| 67 | 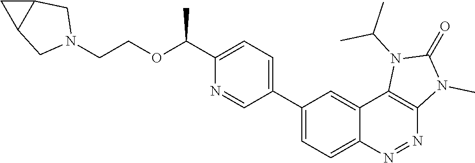 [SMILES: CC(C)n1c(=O)n(C)c2nnc3ccc(-c4ccc([C@H](C)OCCN5CC6CC6C5)nc4)cc3c21] |
| | 8-(6-((1S)-1-(2-(3-azabicyclo[3.1.0]hexan-3-yl)ethoxy)ethyl)pyridin-3- |
| | yl)-1-isopropyl-3-methyl-1H-imidazo[4,5-c]cinnolin-2(3H)-one |
| 68 | 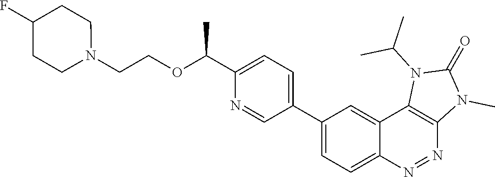 [SMILES: CC(C)n1c(=O)n(C)c2nnc3ccc(-c4ccc([C@H](C)OCCN5CCC(F)CC5)nc4)cc3c21] |
| | (S)-8-(6-(1-(2-(4-fluoropiperidin-1-yl)ethoxy)ethyl)pyridin-3-yl)-1-isopropyl- |
| | 3-methyl-1H-imidazo[4,5-c]cinnolin-2(3H)-one |
| 69 | 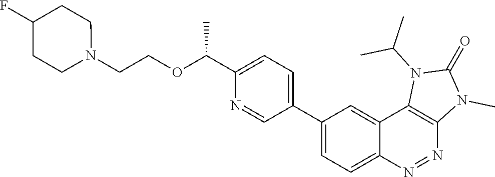 [SMILES: CC(C)n1c(=O)n(C)c2nnc3ccc(-c4ccc([C@@H](C)OCCN5CCC(F)CC5)nc4)cc3c21] |
| | (R)-8-(6-(1-(2-(4-fluoropiperidin-1-yl)ethoxy)ethyl)pyridin-3-yl)-1-isopropyl- |
| | 3-methyl-1H-imidazo[4,5-c]cinnolin-2(3H)-one |
| 70 | 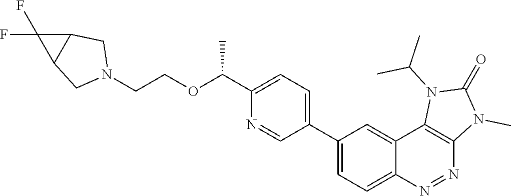 [SMILES: CC(C)n1c(=O)n(C)c2nnc3ccc(-c4ccc([C@@H](C)OCCN5CC6C(C5)C6(F)F)nc4)cc3c21] |
| | 8-(6-((1S)-1-(2-(6,6-difluoro-3-azabicyclo[3.1.0]hexan-3-yl)ethoxy)ethyl)- |
| | pyridin-3-yl)-1-isopropyl-3-methyl-1H-imidazo[4,5-c]cinnolin-2(3H)-one |
| 71 | 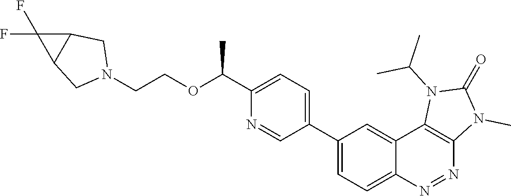 [SMILES: CC(C)n1c(=O)n(C)c2nnc3ccc(-c4ccc([C@H](C)OCCN5CC6C(C5)C6(F)F)nc4)cc3c21] |
| | 8-(6-((1R)-1-(2-(6,6-difluoro-3-azabicyclo[3.1.0]hexan-3-yl)ethoxy)ethyl)- |
| | pyridin-3-yl)-1-isopropyl-3-methyl-1H-imidazo[4,5-c]cinnolin-2(3H)-one |
| 72 | 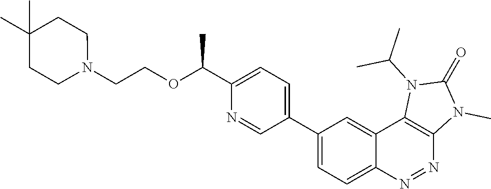 |
| | (S)-8-(6-(1-(2-(4,4-dimethylpiperidin-1-yl)ethoxy)ethyl)pyridin-3-yl)-1- |
| | isopropyl-3-methyl-1H-imidazo[4,5-c]cinnolin-2(3H)-one |
| 73 | 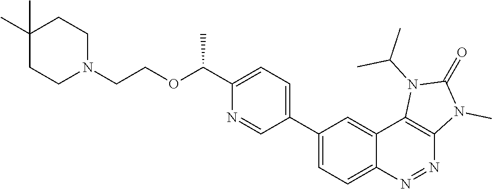 |
| | (R)-8-(6-(1-(2-(4,4-dimethylpiperidin-1-yl)ethoxy)ethyl)pyridin-3-yl)-1- |
| | isopropyl-3-methyl-1H-imidazo[4,5-c]cinnolin-2(3H)-one |
| 74 | 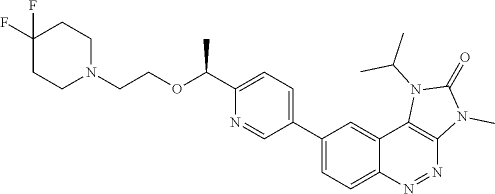 [SMILES: CC(C)n1c(=O)n(C)c2nnc3ccc(-c4ccc([C@H](C)OCCN5CCC(F)(F)CC5)nc4)cc3c21] |
| | (S)-8-(6-(1-(2-(4,4-difluoropiperidin-1-yl)ethoxy)ethyl)pyridin-3-yl)-1- |
| | isopropyl-3-methyl-1H-imidazo[4,5-c]cinnolin-2(3H)-one |
| 75 | 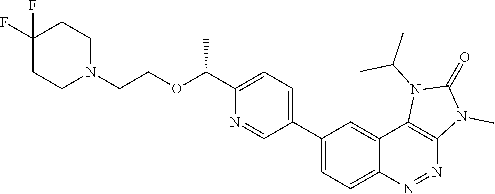 [SMILES: CC(C)n1c(=O)n(C)c2nnc3ccc(-c4ccc([C@@H](C)OCCN5CCC(F)(F)CC5)nc4)cc3c21] |
| | (R)-8-(6-(1-(2-(4,4-difluoropiperidin-1-yl)ethoxy)ethyl)pyridin-3-yl)-1- |
| | isopropyl-3-methyl-1H-imidazo[4,5-c]cinnolin-2(3H)-one |
| 76 | 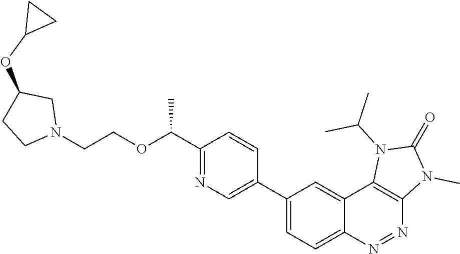 [SMILES: CC(C)n1c(=O)n(C)c2nnc3ccc(-c4ccc([C@@H](C)OCCN5CC[C@@H](OC6CC6)C5)nc4)cc3c21] |
| | 8-(6-((R)-1-(2-((R)-3-cyclopropoxypyrrolidin-1-yl)ethoxy)ethyl)pyridin- |
| | 3-yl)-1-isopropyl-3-methyl-1H-imidazo[4,5-c]cinnolin-2(3H)-one |
| 77 | 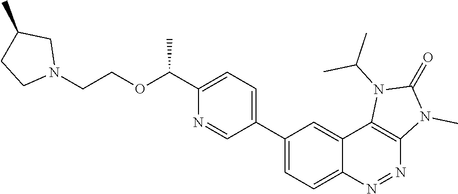 [SMILES: CC(C)n1c(=O)n(C)c2nnc3ccc(-c4ccc([C@@H](C)OCCN5CC[C@@H](C)C5)nc4)cc3c21] |
| | 1-isopropyl-3-methyl-8-(6-((R)-1-(2-((R)-3-methylpyrrolidin-1-yl)- |
| | ethoxy)ethyl)pyridin-3-yl)-1H-imidazo[4,5-c]cinnolin-2(3H)-one |
| 78 | 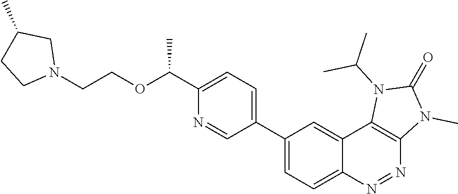 [SMILES: CC(C)n1c(=O)n(C)c2nnc3ccc(-c4ccc([C@@H](C)OCCN5CC[C@H](C)C5)nc4)cc3c21] |
| | 1-isopropyl-3-methyl-8-(6-((R)-1-(2-((S)-3-methylpyrrolidin-1-yl)- |
| | ethoxy)ethyl)pyridin-3-yl)-1H-imidazo[4,5-c]cinnolin-2(3H)-one |
| 79 | 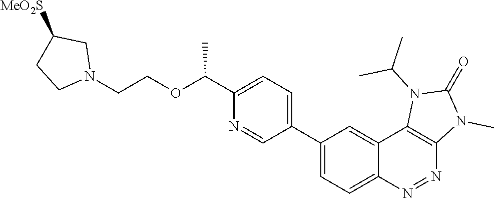 [SMILES: CC(C)n1c(=O)n(C)c2nnc3ccc(-c4ccc([C@@H](C)OCCN5CC[C@@H](S(C)(=O)=O)C5)nc4)cc3c21] |
| | 1-isopropyl-3-methyl-8-(6-((R)-1-(2-((R)-3-(methylsulfonyl)pyrrolidin-1- |
| | yl)ethoxy)ethyl)pyridin-3-yl)-1H-imidazo[4,5-c]cinnolin-2(3H)-one |
| 80 | 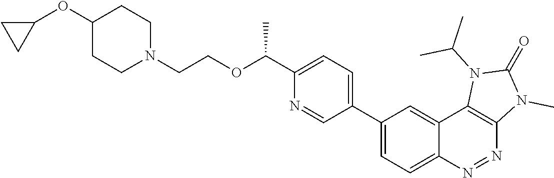 [SMILES: CC(C)n1c(=O)n(C)c2nnc3ccc(-c4ccc([C@@H](C)OCCN5CCC(OC6CC6)CC5)nc4)cc3c21] |
| | 8-(6-((R)-1-(2-((R)-3-cyclopropoxypyrrolidin-1-yl)ethoxy)ethyl)pyridin-3-yl)-3- |
| | methyl-1-(tetrahydro-2H-pyran-4-yl)-1H-imidazo[4,5-c]cinnolin-2(3H)-one |
| 81 | 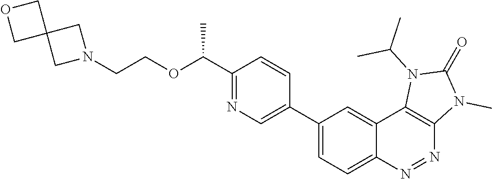 [SMILES: CC(C)n1c(=O)n(C)c2nnc3ccc(-c4ccc([C@@H](C)OCCN5CC6(COC6)C5)nc4)cc3c21] |
| | (R)-8-(6-(1-(2-(2-oxa-6-azaspiro[3.3]heptan-6-yl)ethoxy)ethyl)pyridin-3- |
| | yl)-1-isopropyl-3-methyl-1H-imidazo[4,5-c]cinnolin-2(3H)-one |
| 82 | 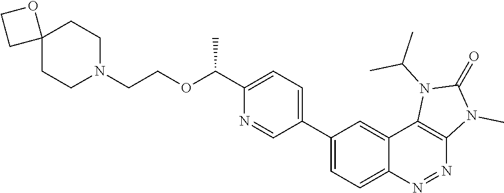 [SMILES: CC(C)n1c(=O)n(C)c2nnc3ccc(-c4ccc([C@@H](C)OCCN5CCC6(CCO6)CC5)nc4)cc3c21] |
| | (R)-8-(6-(1-(2-(1-oxa-7-azaspiro[3.5]nonan-7-yl)ethoxy)ethyl)pyridin-3-yl)- |
| | 1-isopropyl-3-methyl-1H-imidazo[4,5-c]cinnolin-2(3H)-one |
| 83 | 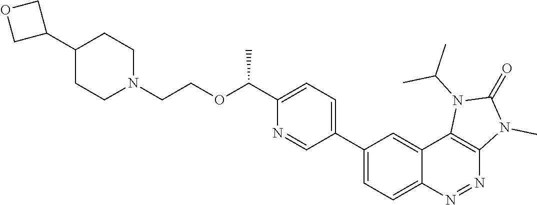 [SMILES: CC(C)n1c(=O)n(C)c2nnc3ccc(-c4ccc([C@@H](C)OCCN5CCC(C6COC6)CC5)nc4)cc3c21] |
| | (R)-1-isopropyl-3-methyl-8-(6-(1-(2-(4-(oxetan-3-yl)piperidin-1-yl)ethoxy)ethyl)- |
| | pyridin-3-yl)-1H-imidazo[4,5-c]cinnolin-2(3H)-one |
| 84 | 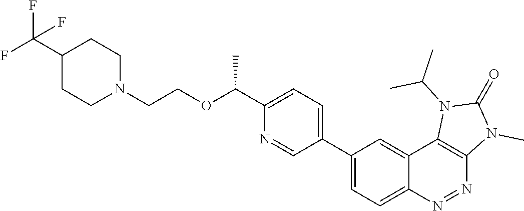 [SMILES: CC(C)n1c(=O)n(C)c2nnc3ccc(-c4ccc([C@@H](C)OCCN5CCC(C(F)(F)F)CC5)nc4)cc3c21] |
| | (R)-1-isopropyl-3-methyl-8-(6-(1-(2-(4-(trifluoromethyl)piperidin-1-yl)ethoxy)- |
| | ethyl)pyridin-3-yl)-1H-imidazo[4,5-c]cinnolin-2(3H)-one |
| 85 | 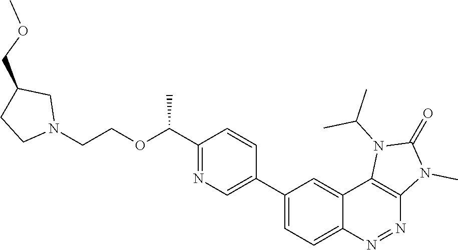 [SMILES: COC[C@@H]1CCN(CCO[C@H](C)c2ccc(-c3ccc4nnc5c(c4c3)n(C(C)C)c(=O)n5C)cn2)C1] |
| | 1-isopropyl-8-(6-((R)-1-(2-((R)-3-(methoxymethyl)pyrrolidin-1-yl)- |
| | ethoxy)ethyl)pyridin-3-yl)-3-methyl-1H-imidazo[4,5-c]cinnolin- |
| | 2(3H)-one |
| 86 | 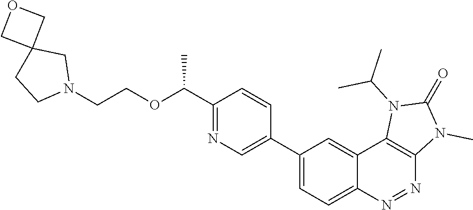 [SMILES: CC(C)n1c(=O)n(C)c2nnc3ccc(-c4ccc([C@@H](C)OCCN5CCC6(COC6)C5)nc4)cc3c21] |
| | (R)-8-(6-(1-(2-(2-oxa-6-azaspiro[3.4]octan-6-yl)ethoxy)ethyl)pyridin-3- |
| | yl)-1-isopropyl-3-methyl-1H-imidazo[4,5-c]cinnolin-2(3H)-one |
| 87 | 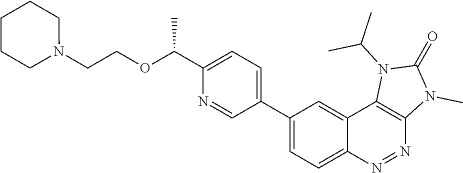 [SMILES: CC(C)n1c(=O)n(C)c2nnc3ccc(-c4ccc([C@@H](C)OCCN5CCCCC5)nc4)cc3c21] |
| | (R)-1-isopropyl-3-methyl-8-(6-(1-(2-(piperidin-1-yl)ethoxy)ethyl)pyridin- |
| | 3-yl)-1H-imidazo[4,5-c]cinnolin-2(3H)-one |
| 88 | 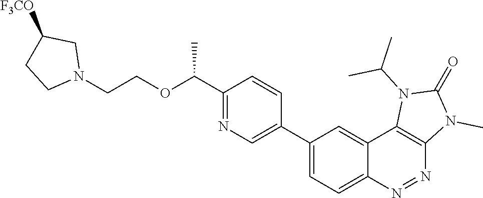 [SMILES: CC(C)n1c(=O)n(C)c2nnc3ccc(-c4ccc([C@@H](C)OCCN5CC[C@@H](OC(F)(F)F)C5)nc4)cc3c21] |
| | 1-isopropyl-3-methyl-8-(6-((R)-1-(2-((R)-3-(trifluoromethoxy)pyrrolidin- |
| | 1-yl)ethoxy)ethyl)pyridin-3-yl)-1H-imidazo[4,5-c]cinnolin-2(3H)-one |
| 89 | 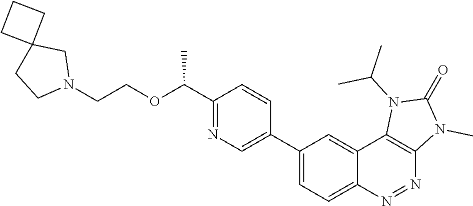 [SMILES: CC(C)n1c(=O)n(C)c2nnc3ccc(-c4ccc([C@@H](C)OCCN5CCC6(CCC6)C5)nc4)cc3c21] |
| | (R)-8-(6-(1-(2-(6-azaspiro[3.4]octan-6-yl)ethoxy)ethyl)pyridin-3-yl)-1- |
| | isopropyl-3-methyl-1H-imidazo[4,5-c]cinnolin-2(3H)-one |
| 90 | 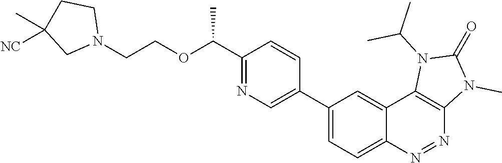 |
| | 1-(2-((R)-1-(5-(1-isopropyl-3-methyl-2-oxo-2,3-dihydro-1H-imidazo[4,5- |
| | c]cinnolin-8-yl)pyridin-2-yl)ethoxy)ethyl)-3-methylpyrrolidine- |
| | 3-carbonitrile |
| 91 | 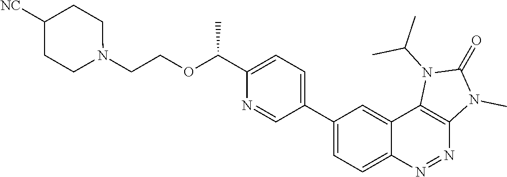 [SMILES: CC(C)n1c(=O)n(C)c2nnc3ccc(-c4ccc([C@@H](C)OCCN5CCC(C#N)CC5)nc4)cc3c21] |
| | (R)-1-(2-(1-(5-(1-isopropyl-3-methyl-2-oxo-2,3-dihydro-1H-imidazo[4,5- |
| | c]cinnolin-8-yl)pyridin-2-yl)ethoxy)ethyl)piperidine-4-carbonitrile |
| 92 | 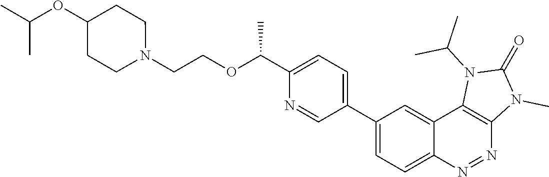 [SMILES: CC(C)OC1CCN(CCO[C@H](C)c2ccc(-c3ccc4nnc5c(c4c3)n(C(C)C)c(=O)n5C)cn2)CC1] |
| | (R)-8-(6-(1-(2-(4-isopropoxypiperidin-1-yl)ethoxy)ethyl)pyridin-3-yl)-1-isopropyl- |
| | 3-methyl-1H-imidazo[4,5-c]cinnolin-2(3H)-one |
| 93 | 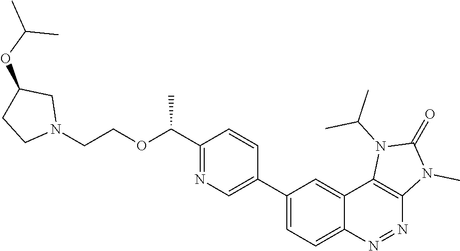 [SMILES: CC(C)O[C@@H]1CCN(CCO[C@H](C)c2ccc(-c3ccc4nnc5c(c4c3)n(C(C)C)c(=O)n5C)cn2)C1] |
| | 8-(6-((R)-1-(2-((R)-3-isopropoxypyrrolidin-1-yl)ethoxy) ethyl)pyridin-3- |
| | yl)-1-isopropyl-3-methyl-1H-imidazo[4,5-c]cinnolin-2(3H)-one |
| 94 | 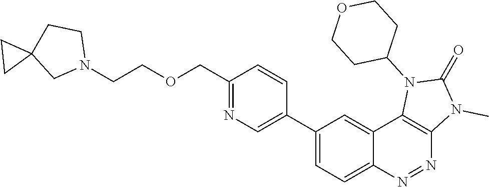 [SMILES: Cn1c(=O)n(C2CCOCC2)c2c3cc(-c4ccc(COCCN5CCC6(CC6)C5)nc4)ccc3nnc21] |
| | 8-(6-((2-(5-azaspiro[2.4]heptan-5-yl)ethoxy)methyl)pyridin-3-yl)-3-methyl- |
| | 1-(tetrahydro-2H-pyran-4-yl)-1H-imidazo[4,5-c]cinnolin-2(3H)-one |
| 95 | 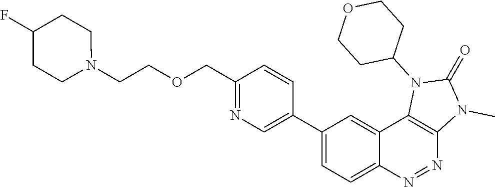 [SMILES: Cn1c(=O)n(C2CCOCC2)c2c3cc(-c4ccc(COCCN5CCC(F)CC5)nc4)ccc3nnc21] |
| | 8-(6-((2-(4-fluoropiperidin-1-yl)ethoxy)methyl)pyridin-3-yl)-3-methyl-1- |
| | (tetrahydro-2H-pyran-4-yl)-1H-imidazo[4,5-c]cinnolin-2(3H)-one |
| 96 | 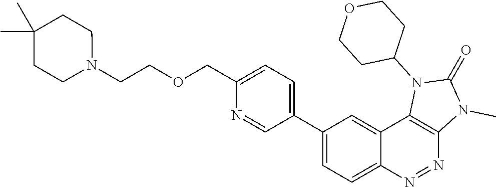 |
| | 8-(6-((2-(4,4-dimethylpiperidin-1-yl)ethoxy)methyl)pyridin-3-yl)-3-methyl-1- |
| | (tetrahydro-2H-pyran-4-yl)-1H-imidazo[4,5-c]cinnolin-2(3H)-one |
| 97 | 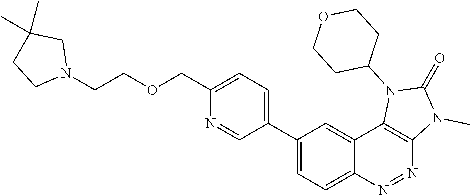 |
| | 8-(6-((2-(3,3-dimethylpyrrolidin-1-yl)ethoxy)methyl)pyridin-3-yl)-3-meth- |
| | yl-1-(tetrahydro-2H-pyran-4-yl)-1H-imidazo[4,5-c]cinnolin-2(3H)-one |
| 98 | 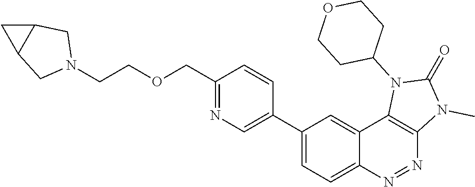 [SMILES: Cn1c(=O)n(C2CCOCC2)c2c3cc(-c4ccc(COCCN5CC6CC6C5)nc4)ccc3nnc21] |
| | 8-(6-((2-(3-azabicyclo[3.1.0]hexan-3-yl)ethoxy)methyl)pyridin-3-yl)-3-meth- |
| | yl-1-(tetrahydro-2H-pyran-4-yl)-1H-imidazo[4,5-c]cinnolin-2(3H)-one |
| 99 | 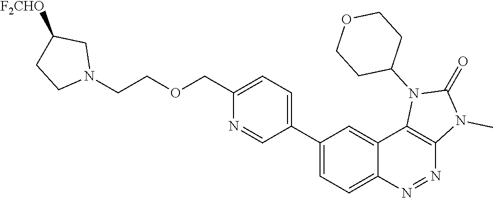 |
| | (R)-8-(6-((2-(3-(difluoromethoxy)pyrrolidin-1-yl)ethoxy)methyl)pyridin-3- |
| | yl)-3-methyl-1-(tetrahydro-2H-pyran-4-yl)-1H-imidazo[4,5-c]cinnolin- |
| | 2(3H)-one |
| 100 | 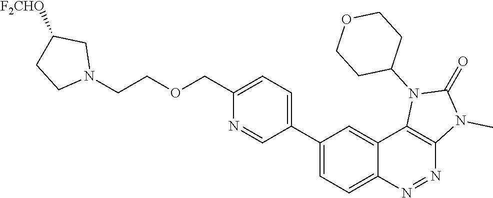 |
| | (S)-8-(6-((2-(3-(difluoromethoxy)pyrrolidin-1-yl)ethoxy)methyl)pyridin- |
| | 3-yl)-3-methyl-1-(tetrahydro-2H-pyran-4-yl)-1H-imidazo[4,5-c]- |
| | cinnolin-2(3H)-one |
| 101 | 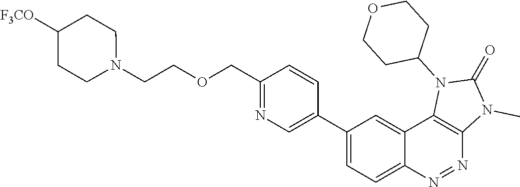 [SMILES: Cn1c(=O)n(C2CCOCC2)c2c3cc(-c4ccc(COCCN5CCC(OC(F)(F)F)CC5)nc4)ccc3nnc21] |
| | 3-methyl-1-(tetrahydro-2H-pyran-4-yl)-8-(6-((2-(4-(trifluoromethoxy)- |
| | piperidin-1-yl)ethoxy)methyl)pyridin-3-yl)-1H-imidazo[4,5-c]- |
| | cinnolin-2(3H)-one |
| 102 | 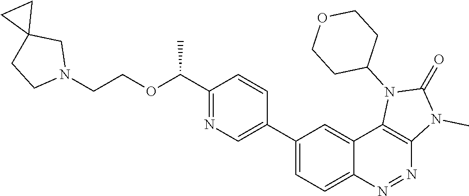 [SMILES: C[C@@H](OCCN1CCC2(CC2)C1)c1ccc(-c2ccc3nnc4c(c3c2)n(C2CCOCC2)c(=O)n4C)cn1] |
| | (R)-8-(6-(1-(2-(5-azaspiro[2.4]heptan-5-yl)ethoxy)ethyl)pyridin-3-yl)-3- |
| | methyl-1-(tetrahydro-2H-pyran-4-yl)-1H-imidazo[4,5-c]cinnolin- |
| | 2(3H)-one |
| 103 | 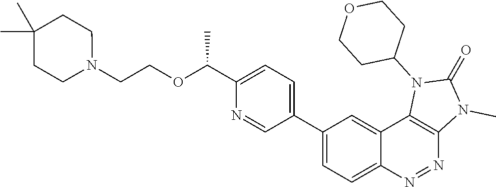 |
| | (R)-8-(6-(1-(2-(4,4-dimethylpiperidin-1-yl)ethoxy)ethyl)pyridin-3-yl)-3- |
| | methyl-1-(tetrahydro-2H-pyran-4-yl)-1H-imidazo[4,5-c]cinnolin- |
| | 2(3H)-one |
| 104 | 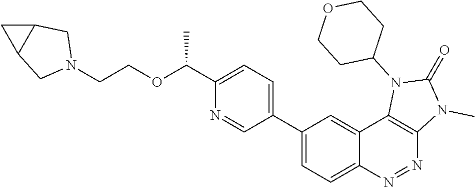 [SMILES: C[C@@H](OCCN1CC2CC2C1)c1ccc(-c2ccc3nnc4c(c3c2)n(C2CCOCC2)c(=O)n4C)cn1] |
| | 8-(6-((1R)-1-(2-(3-azabicyclo[3.1.0]hexan-3-yl)ethoxy)ethyl)pyridin-3- |
| | yl)-3-methyl-1-(tetrahydro-2H-pyran-4-yl)-1H-imidazo[4,5-c]cinnolin- |
| | 2(3H)-one |
| 105 | 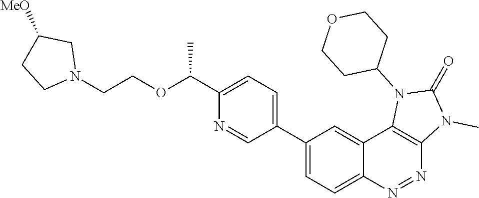 [SMILES: CO[C@H]1CCN(CCO[C@H](C)c2ccc(-c3ccc4nnc5c(c4c3)n(C3CCOCC3)c(=O)n5C)cn2)C1] |
| | 8-(6-((R)-1-(2-((S)-3-methoxypyrrolidin-1-yl)ethoxy)ethyl)pyridin-3-yl)- |
| | 3-methyl-1-(tetrahydro-2H-pyran-4-yl)-1H-imidazo[4,5-c]cinnolin- |
| | 2(3H)-one |
| 106 | 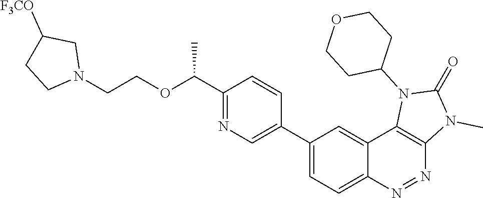 [SMILES: C[C@@H](OCCN1CCC(OC(F)(F)F)C1)c1ccc(-c2ccc3nnc4c(c3c2)n(C2CCOCC2)c(=O)n4C)cn1] |
| | 3-methyl-1-(tetrahydro-2H-pyran-4-yl)-8-(6-((1R)-1-(2-(3-(trifluoro- |
| | methoxy)pyrrolidin-1-yl)ethoxy)ethyl)pyridin-3-yl)-1H-imidazo- |
| | [4,5-c]cinnolin-2(3H)-one |
| 107 | 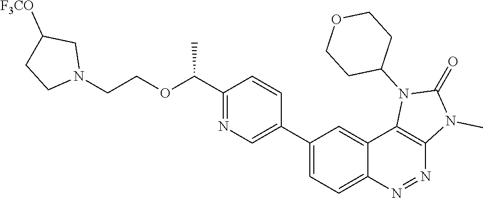 [SMILES: C[C@@H](OCCN1CCC(OC(F)(F)F)C1)c1ccc(-c2ccc3nnc4c(c3c2)n(C2CCOCC2)c(=O)n4C)cn1] |
| | (R)-8-(6-(1-(2-(6-azaspiro[2.5]octan-6-yl)ethoxy)ethyl)pyridin-3-yl)-3- |
| | methyl-1-(tetrahydro-2H-pyran-4-yl)-1H-imidazo[4,5-c]cinnolin- |
| | 2(3H)-one |
| 108 | 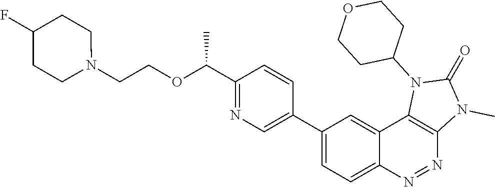 [SMILES: C[C@@H](OCCN1CCC(F)CC1)c1ccc(-c2ccc3nnc4c(c3c2)n(C2CCOCC2)c(=O)n4C)cn1] |
| | 8-(6-((R)-1-(2-(4-fluoropiperidin-1-yl)ethoxy)ethyl)pyridin-3-yl)-3-methyl- |
| | 1-(tetrahydro-2H-pyran-4-yl)-1,3,3a,9b-tetrahydro-2H-imidazo[4,5- |
| | c]cinnolin-2-one |
| 109 | 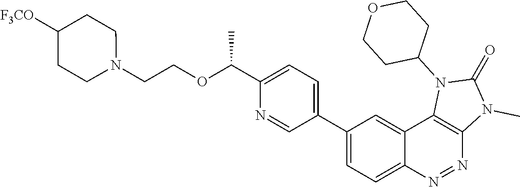 [SMILES: C[C@@H](OCCN1CCC(OC(F)(F)F)CC1)c1ccc(-c2ccc3nnc4c(c3c2)n(C2CCOCC2)c(=O)n4C)cn1] |
| | (R)-3-methyl-1-(tetrahydro-2H-pyran-4-yl)-8-(6-(1-(2-(4-(trifluoro- |
| | methoxy)piperidin-1-yl)ethoxy)ethyl)pyridin-3-yl)-1H-imidazo[4,5- |
| | c]cinnolin-2(3H)-one |
| 110 | 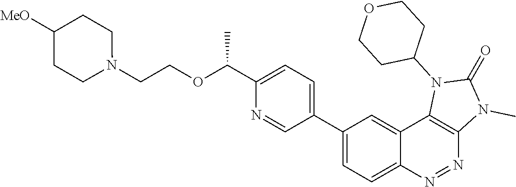 [SMILES: COC1CCN(CCO[C@H](C)c2ccc(-c3ccc4nnc5c(c4c3)n(C3CCOCC3)c(=O)n5C)cn2)CC1] |
| | (R)-8-(6-(1-(2-(4-methoxypiperidin-1-yl)ethoxy)ethyl)pyridin-3-yl)-3-methyl- |
| | 1-(tetrahydro-2H-pyran-4-yl)-1H-imidazo[4,5-c]cinnolin-2(3H)-one |
| 111 | 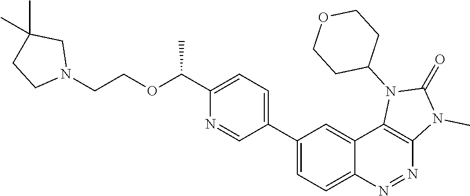 |
| | (R)-8-(6-(1-(2-(3,3-dimethylpyrrolidin-1-yl)ethoxy)ethyl)pyridin-3-yl)- |
| | 3-methyl-1-(tetrahydro-2H-pyran-4-yl)-1H-imidazo[4,5-c]cinnolin- |
| | 2(3H)-one |
| 112 | 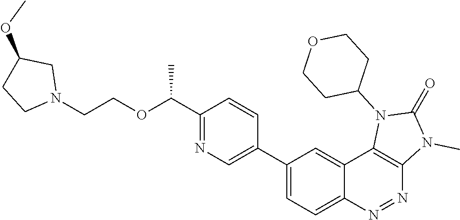 [SMILES: CO[C@@H]1CCN(CCO[C@H](C)c2ccc(-c3ccc4nnc5c(c4c3)n(C3CCOCC3)c(=O)n5C)cn2)C1] |
| | 8-(6-((R)-1-(2-((R)-3-methoxypyrrolidin-1-yl)ethoxy)ethyl)pyridin-3-yl)- |
| | 3-methyl-1-(tetrahydro-2H-pyran-4-yl)-1H-imidazo[4,5-c]cinnolin- |
| | 2(3H)-one |
| 113 | 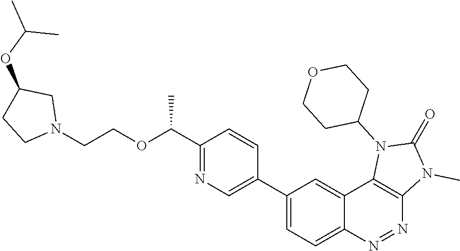 [SMILES: CC(C)O[C@@H]1CCN(CCO[C@H](C)c2ccc(-c3ccc4nnc5c(c4c3)n(C3CCOCC3)c(=O)n5C)cn2)C1] |
| | 8-(6-((R)-1-(2-((R)-3-isopropoxypyrrolidin-1-yl)ethoxy)ethyl)pyridin-3-yl)- |
| | 3-methyl-1-(tetrahydro-2H-pyran-4-yl)-1H-imidazo[4,5-c]cinnolin- |
| | 2(3H)-one |
| 114 | 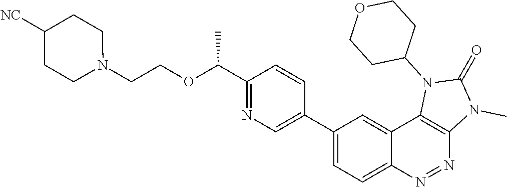 [SMILES: C[C@@H](OCCN1CCC(C#N)CC1)c1ccc(-c2ccc3nnc4c(c3c2)n(C2CCOCC2)c(=O)n4C)cn1] |
| | (R)-1-(2-(1-(5-(3-methyl-2-oxo-1-(tetrahydro-2H-pyran-4-yl)-2,3-dihydro-1H- |
| | imidazo[4,5-c]cinnolin-8-yl)pyridin-2-yl)ethoxy)ethyl)piperidine- |
| | 4-carbonitrile |
| 115 | 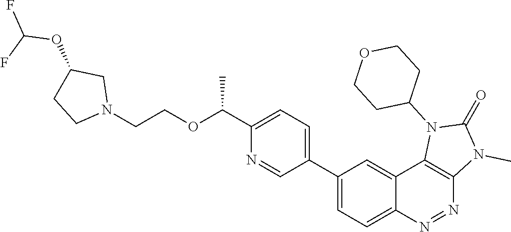 [SMILES: C[C@@H](OCCN1CC[C@H](OC(F)F)C1)c1ccc(-c2ccc3nnc4c(c3c2)n(C2CCOCC2)c(=O)n4C)cn1] |
| | 8-(6-((R)-1-(2-((S)-3-(difluoromethoxy)pyrrolidin-1-yl)ethoxy)ethyl)pyridin- |
| | 3-yl)-3-methyl-1-(tetrahydro-2H-pyran-4-yl)-1,3-dihydro-2H-imidazo- |
| | [4,5-c]cinnolin-2-one |
| 116 | 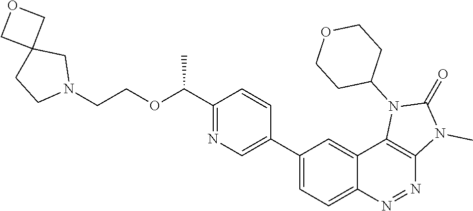 [SMILES: C[C@@H](OCCN1CCC2(COC2)C1)c1ccc(-c2ccc3nnc4c(c3c2)n(C2CCOCC2)c(=O)n4C)cn1] |
| | (R)-8-(6-(1-(2-(2-oxa-6-azaspiro[3.4]octan-6-yl)ethoxy)ethyl)pyridin-3- |
| | yl)-3-methyl-1-(tetrahydro-2H-pyran-4-yl)-1H-imidazo[4,5-c]cinnolin- |
| | 2(3H)-one |
| 117 | 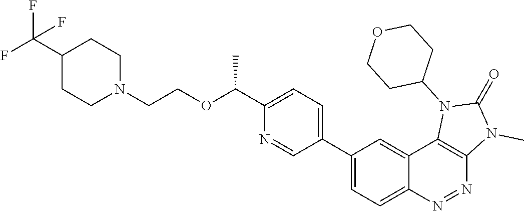 [SMILES: C[C@@H](OCCN1CCC(C(F)(F)F)CC1)c1ccc(-c2ccc3nnc4c(c3c2)n(C2CCOCC2)c(=O)n4C)cn1] |
| | (R)-3-methyl-1-(tetrahydro-2H-pyran-4-yl)-8-(6-(1-(2-(4-(trifluoromethyl)- |
| | piperidin-1-yl)ethoxy)ethyl)pyridin-3-yl)-1H-imidazo[4,5-c]cinnolin- |
| | 2(3H)-one |
| 118 | 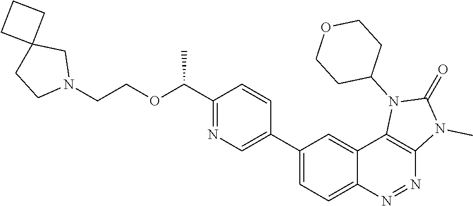 [SMILES: C[C@@H](OCCN1CCC2(CCC2)C1)c1ccc(-c2ccc3nnc4c(c3c2)n(C2CCOCC2)c(=O)n4C)cn1] |
| | (R)-8-(6-(1-(2-(6-azaspiro[3.4]octan-6-yl)ethoxy)ethyl)pyridin-3-yl)-3-meth- |
| | yl-1-(tetrahydro-2H-pyran-4-yl)-1H-imidazo[4,5-c]cinnolin-2(3H)-one |
| 119 | 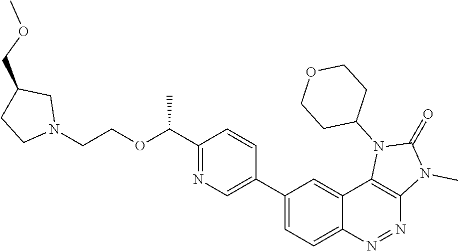 [SMILES: COC[C@@H]1CCN(CCO[C@H](C)c2ccc(-c3ccc4nnc5c(c4c3)n(C3CCOCC3)c(=O)n5C)cn2)C1] |
| | 8-(6-((R)-1-(2-((R)-3-(methoxymethyl)pyrrolidin-1-yl)ethoxy)ethyl)- |
| | pyridin-3-yl)-3-methyl-1-(tetrahydro-2H-pyran-4-yl)-1H-imidazo- |
| | [4,5-c]cinnolin-2(3H)-one |
| 120 | 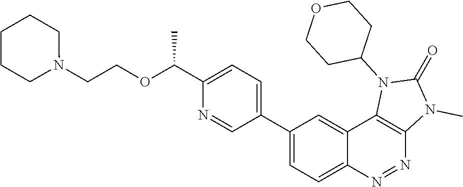 [SMILES: C[C@@H](OCCN1CCCCC1)c1ccc(-c2ccc3nnc4c(c3c2)n(C2CCOCC2)c(=O)n4C)cn1] |
| | (R)-3-methyl-8-(6-(1-(2-(piperidin-1-yl)ethoxy)ethyl)pyridin-3-yl)-1- |
| | (tetrahydro-2H-pyran-4-yl)-1H-imidazo[4,5-c]cinnolin-2(3H)-one |
| 121 | 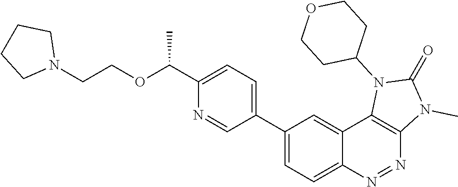 [SMILES: C[C@@H](OCCN1CCCC1)c1ccc(-c2ccc3nnc4c(c3c2)n(C2CCOCC2)c(=O)n4C)cn1] |
| | (R)-3-methyl-8-(6-(1-(2-(pyrrolidin-1-yl)ethoxy)ethyl)pyridin-3-yl)-1- |
| | (tetrahydro-2H-pyran-4-yl)-1H-imidazo[4,5-c]cinnolin-2(3H)-one |
| 122 | 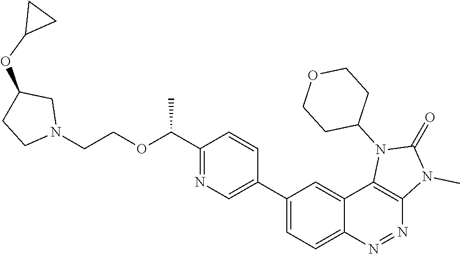 [SMILES: C[C@@H](OCCN1CC[C@@H](OC2CC2)C1)c1ccc(-c2ccc3nnc4c(c3c2)n(C2CCOCC2)c(=O)n4C)cn1] |
| | 8-(6-((R)-1-(2-((R)-3-cyclopropoxypyrrolidin-1-yl)ethoxy)ethyl)- |
| | pyridin-3-yl)-3-methyl-1-(tetrahydro-2H-pyran-4-yl)-1H- |
| | imidazo[4,5-c]cinnolin-2(3H)-one |
| 123 | 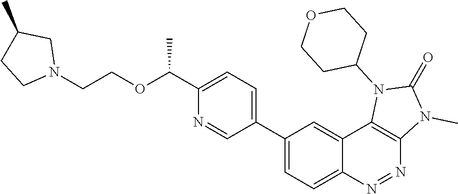 [SMILES: C[C@@H]1CCN(CCO[C@H](C)c2ccc(-c3ccc4nnc5c(c4c3)n(C3CCOCC3)c(=O)n5C)cn2)C1] |
| | 3-methyl-8-(6-((R)-1-(2-((R)-3-methylpyrrolidin-1-yl)ethoxy)ethyl)- |
| | pyridin-3-yl)-1-(tetrahydro-2H-pyran-4-yl)-1H-imidazo[4,5- |
| | c]cinnolin-2(3H)-one |
| 124 | 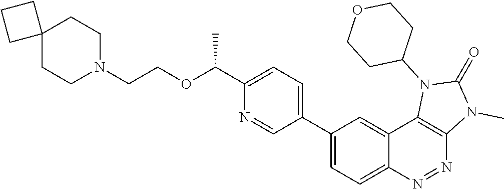 [SMILES: C[C@@H](OCCN1CCC2(CCC2)CC1)c1ccc(-c2ccc3nnc4c(c3c2)n(C2CCOCC2)c(=O)n4C)cn1] |
| | (R)-8-(6-(1-(2-(7-azaspiro[3.5]nonan-7-yl)ethoxy)ethyl)pyridin-3-yl)-3-meth- |
| | yl-1-(tetrahydro-2H-pyran-4-yl)-1H-imidazo[4,5-c]cinnolin-2(3H)-one |
| 125 | 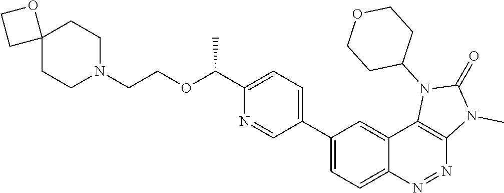 [SMILES: C[C@@H](OCCN1CCC2(CCO2)CC1)c1ccc(-c2ccc3nnc4c(c3c2)n(C2CCOCC2)c(=O)n4C)cn1] |
| | (R)-8-(6-(1-(2-(1-oxa-7-azaspiro[3.5]nonan-7-yl)ethoxy)ethyl)pyridin-3-yl)- |
| | 3-methyl-1-(tetrahydro-2H-pyran-4-yl)-1H-imidazo[4,5-c]cinnolin- |
| | 2(3H)-one |
| 126 | 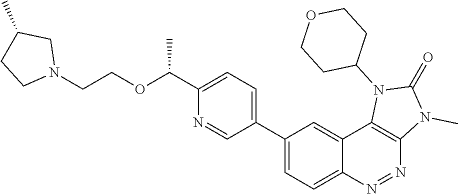 [SMILES: C[C@H]1CCN(CCO[C@H](C)c2ccc(-c3ccc4nnc5c(c4c3)n(C3CCOCC3)c(=O)n5C)cn2)C1] |
| | 3-methyl-8-(6-((R)-1-(2-((S)-3-methylpyrrolidin-1-yl)ethoxy)ethyl)- |
| | pyridin-3-yl)-1-(tetrahydro-2H-pyran-4-yl)-1H-imidazo[4,5- |
| | c]cinnolin-2(3H)-one |
| 127 | 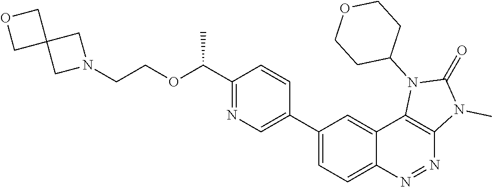 [SMILES: C[C@@H](OCCN1CC2(COC2)C1)c1ccc(-c2ccc3nnc4c(c3c2)n(C2CCOCC2)c(=O)n4C)cn1] |
| | (R)-8-(6-(1-(2-(2-oxa-6-azaspiro[3.3]heptan-6-yl)ethoxy)ethyl)pyridin-3- |
| | yl)-3-methyl-1-(tetrahydro-2H-pyran-4-yl)-1H-imidazo[4,5-c]cinnolin- |
| | 2(3H)-one |
| 128 | 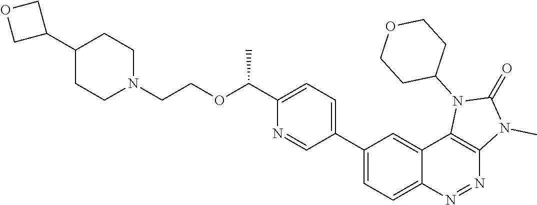 [SMILES: C[C@@H](OCCN1CCC(C2COC2)CC1)c1ccc(-c2ccc3nnc4c(c3c2)n(C2CCOCC2)c(=O)n4C)cn1] |
| | (R)-3-methyl-8-(6-(1-(2-(4-(oxetan-3-yl)piperidin-1-yl)ethoxy)ethyl)pyridin- |
| | 3-yl)-1-(tetrahydro-2H-pyran-4-yl)-1H-imidazo[4,5-c]cinnolin- |
| | 2(3H)-one |
| 129 | 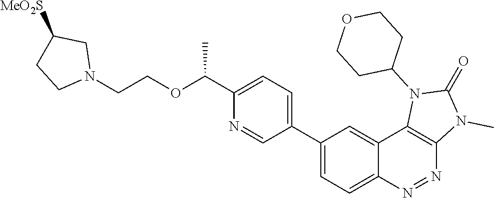 [SMILES: C[C@@H](OCCN1CC[C@@H](S(C)(=O)=O)C1)c1ccc(-c2ccc3nnc4c(c3c2)n(C2CCOCC2)c(=O)n4C)cn1] |
| | 3-methyl-8-(6-((R)-1-(2-((R)-3-(methylsulfonyl)pyrrolidin-1-yl)ethoxy)ethyl)- |
| | pyridin-3-yl)-1-(tetrahydro-2H-pyran-4-yl)-1H-imidazo[4,5-c]cinnolin- |
| | 2(3H)-one |
| 130 | 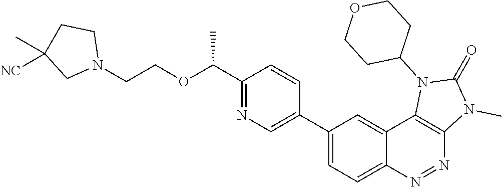 |
| | 3-methyl-1-(2-((R)-1-(5-(3-methyl-2-oxo-1-(tetrahydro-2H-pyran-4-yl)-2,3- |
| | dihydro-1H-imidazo[4,5-c]cinnolin-8-yl)pyridin-2-yl)ethoxy)ethyl) |
| | pyrrolidine-3-carbonitrile |
| 131 | 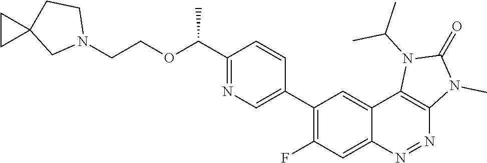 [SMILES: CC(C)n1c(=O)n(C)c2nnc3cc(F)c(-c4ccc([C@@H](C)OCCN5CCC6(CC6)C5)nc4)cc3c21] |
| | 8-(6-((2-(5-azaspiro[2.4]heptan-5-yl)ethoxy)methyl)pyridin-3-yl)-7-fluoro- |
| | 1-isopropyl-3-methyl-1H-imidazo[4,5-c]cinnolin-2(3H)-one |
| 132 | 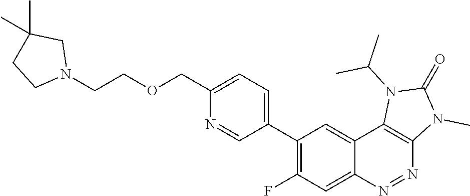 |
| | 8-(6-((2-(3,3-dimethylpyrrolidin-1-yl)ethoxy)methyl)pyridin-3-yl)-7-fluoro- |
| | 1-isopropyl-3-methyl-1H-imidazo[4,5-c]cinnolin-2(3H)-one |
| 133 | 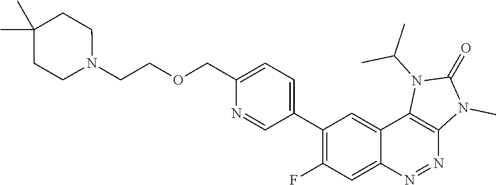 |
| | 8-(6-((2-(4,4-dimethylpiperidin-1-yl)ethoxy)methyl)pyridin-3-yl)-7-fluoro- |
| | 1-isopropyl-3-methyl-1H-imidazo[4,5-c]cinnolin-2(3H)-one |
| 134 | 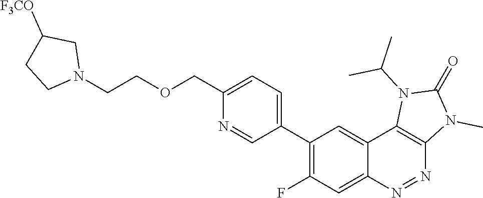 [SMILES: CC(C)n1c(=O)n(C)c2nnc3cc(F)c(-c4ccc(COCCN5CCC(OC(F)(F)F)C5)nc4)cc3c21] |
| | 7-fluoro-1-isopropyl-3-methyl-8-(6-((2-(3-(trifluoromethoxy)pyrrolidin-1- |
| | yl)ethoxy)methyl)pyridin-3-yl)-1,3-dihydro-2H-imidazo[4,5-c]cinnolin- |
| | 2-one |
| 135 | 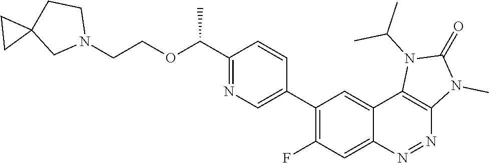 [SMILES: CC(C)n1c(=O)n(C)c2nnc3cc(F)c(-c4ccc([C@@H](C)OCCN5CCC6(CC6)C5)nc4)cc3c21] |
| | (R)-8-(6-(1-(2-(5-azaspiro[2.4]heptan-5-yl)ethoxy)ethyl)pyridin-3-yl)-7-fluoro- |
| | 3-methyl-1-(tetrahydro-2H-pyran-4-yl)-1H-imidazo[4,5-c]cinnolin- |
| | 2(3H)-one |
| 136 | 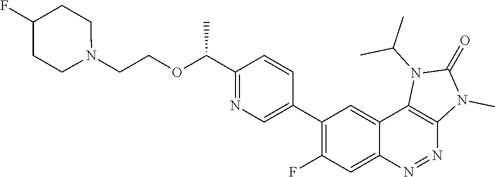 [SMILES: CC(C)n1c(=O)n(C)c2nnc3cc(F)c(-c4ccc([C@@H](C)OCCN5CCC(F)CC5)nc4)cc3c21] |
| | (R)-7-fluoro-8-(6-(1-(2-(4-fluoropiperidin-1-yl)ethoxy)ethyl)pyridin-3-yl)-1- |
| | isopropyl-3-methyl-1,3-dihydro-2H-imidazo[4,5-c]cinnolin-2-one |
| 137 | 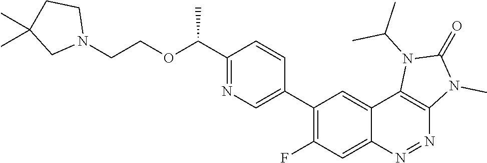 |
| | (R)-8-(6-(1-(2-(3,3-dimethylpyrrolidin-1-yl)ethoxy)ethyl)pyridin-3-yl)-7- |
| | fluoro-1-isopropyl-3-methyl-1H-imidazo[4,5-c]cinnolin-2(3H)-one |
| 138 | 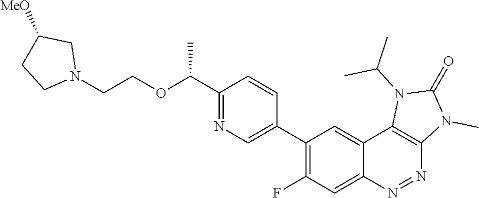 [SMILES: CO[C@H]1CCN(CCO[C@H](C)c2ccc(-c3cc4c(cc3F)nnc3c4n(C(C)C)c(=O)n3C)cn2)C1] |
| | 7-fluoro-1-isopropyl-8-(6-((R)-1-(2-((S)-3-methoxypyrrolidin-1-yl)ethoxy)- |
| | ethyl)pyridin-3-yl)-3-methyl-1H-imidazo[4,5-c]cinnolin-2(3H)-one |
| 139 | 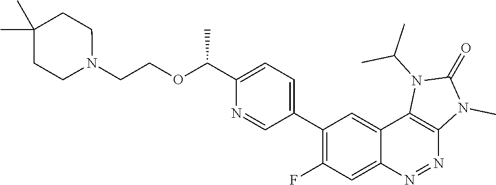 |
| | (R)-8-(6-(1-(2-(4,4-dimethylpiperidin-1-yl)ethoxy)ethyl)pyridin-3-yl)-7-fluoro- |
| | 1-isopropyl-3-methyl-1H-imidazo[4,5-c]cinnolin-2(3H)-one |
| 140 | 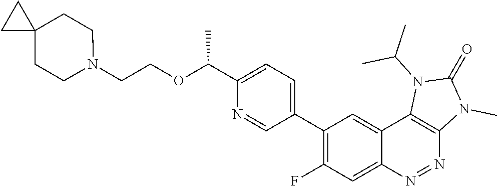 [SMILES: CC(C)n1c(=O)n(C)c2nnc3cc(F)c(-c4ccc([C@@H](C)OCCN5CCC6(CC5)CC6)nc4)cc3c21] |
| | (R)-8-(6-(1-(2-(6-azaspiro[2.5]octan-6-yl)ethoxy)ethyl)pyridin-3-yl)-7-fluoro- |
| | 1-isopropyl-3-methyl-1H-imidazo[4,5-c]cinnolin-2(3H)-one |
| 141 | 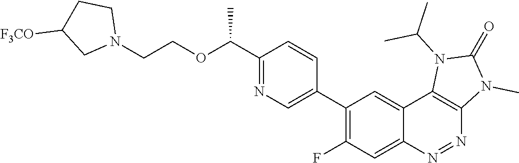 [SMILES: CC(C)n1c(=O)n(C)c2nnc3cc(F)c(-c4ccc([C@@H](C)OCCN5CCC(OC(F)(F)F)C5)nc4)cc3c21] |
| | 7-fluoro-1-isopropyl-3-methyl-8-(6-((1R)-1-(2-(3-(trifluoromethoxy)- |
| | pyrrolidin-1-yl)ethoxy)ethyl)pyridin-3-yl)-1H-imidazo[4,5-c]cinnolin- |
| | 2(3H)-one |
| 142 | 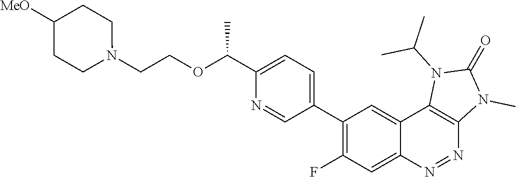 [SMILES: COC1CCN(CCO[C@H](C)c2ccc(-c3cc4c(cc3F)nnc3c4n(C(C)C)c(=O)n3C)cn2)CC1] |
| | (R)-7-fluoro-1-isopropyl-8-(6-(1-(2-(4-methoxypiperidin-1-yl)ethoxy)ethyl)- |
| | pyridin-3-yl)-3-methyl-1H-imidazo[4,5-c]cinnolin-2(3H)-one |
| 143 | 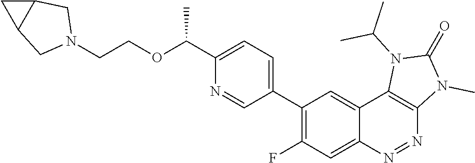 [SMILES: CC(C)n1c(=O)n(C)c2nnc3cc(F)c(-c4ccc([C@@H](C)OCCN5CC6CC6C5)nc4)cc3c21] |
| | 8-(6-((1R)-1-(2-(3-azabicyclo[3.1.0]hexan-3-yl)ethoxy)ethyl)pyridin-3-yl)- |
| | 7-fluoro-1-isopropyl-3-methyl-1H-imidazo[4,5-c]cinnolin-2(3H)-one |
| 144 | 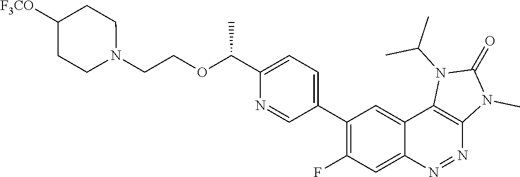 [SMILES: CC(C)n1c(=O)n(C)c2nnc3cc(F)c(-c4ccc([C@@H](C)OCCN5CCC(OC(F)(F)F)CC5)nc4)cc3c21] |
| | (R)-7-fluoro-1-isopropyl-3-methyl-8-(6-(1-(2-(4-(trifluoromethoxy)- |
| | piperidin-1-yl)ethoxy)ethyl)pyridin-3-yl)-1H-imidazo[4,5-c]- |
| | cinnolin-2(3H)-one |
| 145 | 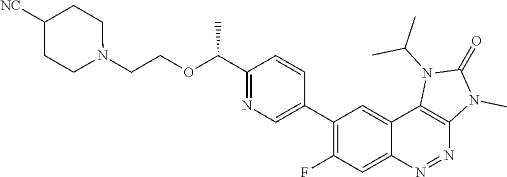 [SMILES: CC(C)n1c(=O)n(C)c2nnc3cc(F)c(-c4ccc([C@@H](C)OCCN5CCC(C#N)CC5)nc4)cc3c21] |
| | (R)-1-(2-(1-(5-(7-fluoro-1-isopropyl-3-methyl-2-oxo-2,3-dihydro-1H-imidazo- |
| | [4,5-c]cinnolin-8-yl)pyridin-2-yl)ethoxy)ethyl)piperidine-4-carbonitrile |
| 146 | 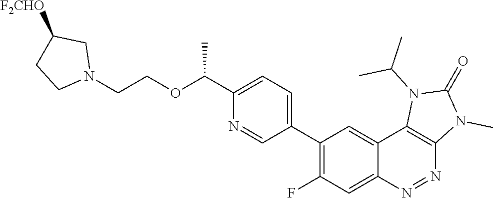 |
| | 8-(6-((R)-1-(2-((R)-3-(difluoromethoxy)pyrrolidin-1-yl)ethoxy)ethyl)- |
| | pyridin-3-yl)-7-fluoro-1-isopropyl-3-methyl-1H-imidazo[4,5-c]- |
| | cinnolin-2(3H)-one |
| 147 | 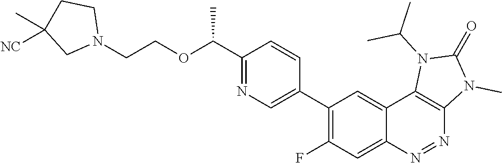 |
| | 1-(2-((R)-1-(5-(7-fluoro-1-isopropyl-3-methyl-2-oxo-2,3-dihydro-1H-imidazo[4,5- |
| | c]cinnolin-8-yl)pyridin-2-yl)ethoxy)ethyl)-3-methylpyrrolidine-3-carbonitrile |
| 148 | 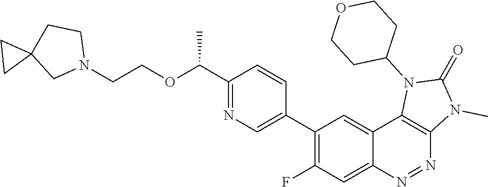 [SMILES: C[C@@H](OCCN1CCC2(CC2)C1)c1ccc(-c2cc3c(cc2F)nnc2c3n(C3CCOCC3)c(=O)n2C)cn1] |
| | (R)-8-(6-(1-(2-(5-azaspiro[2.4]heptan-5-yl)ethoxy)ethyl)pyridin-3-yl)-7-fluoro- |
| | 3-methyl-1-(tetrahydro-2H-pyran-4-yl)-1H-imidazo[4,5-c]cinnolin- |
| | 2(3H)-one |
| 149 | 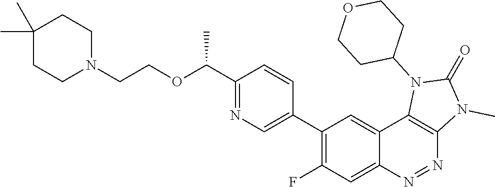 |
| | (R)-8-(6-(1-(2-(4,4-dimethylpiperidin-1-yl)ethoxy)ethyl)pyridin-3-yl)-7-fluoro- |
| | 3-methyl-1-(tetrahydro-2H-pyran-4-yl)-1H-imidazo[4,5-c]cinnolin- |
| | 2(3H)-one |
| 150 | 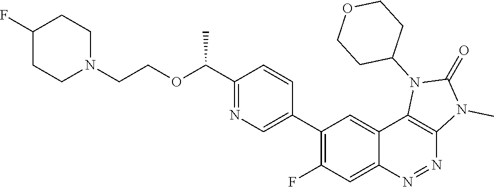 [SMILES: C[C@@H](OCCN1CCC(F)CC1)c1ccc(-c2cc3c(cc2F)nnc2c3n(C3CCOCC3)c(=O)n2C)cn1] |
| | (R)-7-fluoro-8-(6-(1-(2-(4-fluoropiperidin-1-yl)ethoxy)ethyl)pyridin-3-yl)-3- |
| | methyl-1-(tetrahydro-2H-pyran-4-yl)-1H-imidazo[4,5-c]cinnolin-2(3H)-one |
| 151 | 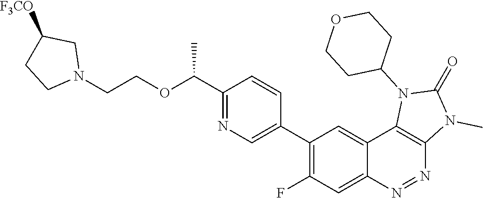 [SMILES: C[C@@H](OCCN1CC[C@@H](OC(F)(F)F)C1)c1ccc(-c2cc3c(cc2F)nnc2c3n(C3CCOCC3)c(=O)n2C)cn1] |
| | 7-fluoro-3-methyl-1-(tetrahydro-2H-pyran-4-yl)-8-(6-((R)-1-(2-((R)-3- |
| | (trifluoromethoxy)pyrrolidin-1-yl)ethoxy)ethyl)pyridin-3-yl)-1,3- |
| | dihydro-2H-imidazo[4,5-c]cinnolin-2-one |
| 152 | 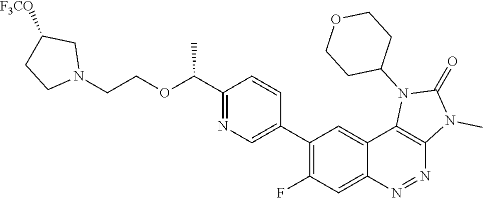 [SMILES: C[C@@H](OCCN1CC[C@H](OC(F)(F)F)C1)c1ccc(-c2cc3c(cc2F)nnc2c3n(C3CCOCC3)c(=O)n2C)cn1] |
| | 7-fluoro-3-methyl-1-(tetrahydro-2H-pyran-4-yl)-8-(6-((R)-1-(2-((S)-3- |
| | (trifluoromethoxy)pyrrolidin-1-yl)ethoxy)ethyl)pyridin-3-yl)-1,3- |
| | dihydro-2H-imidazo[4,5-c]cinnolin-2-one |
| 153 | 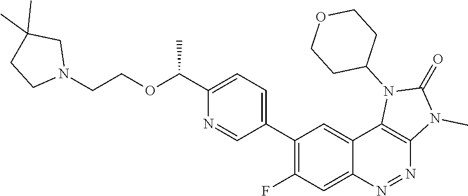 |
| | (R)-8-(6-(1-(2-(3,3-dimethylpyrrolidin-1-yl)ethoxy)ethyl)pyridin-3-yl)-7- |
| | fluoro-3-methyl-1-(tetrahydro-2H-pyran-4-yl)-1H-imidazo[4,5-c]- |
| | cinnolin-2(3H)-one |
| 154 | 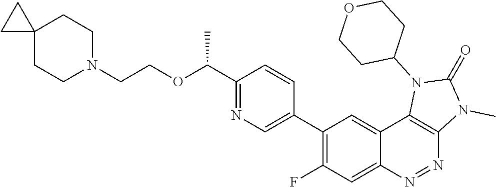 [SMILES: C[C@@H](OCCN1CCC2(CC1)CC2)c1ccc(-c2cc3c(cc2F)nnc2c3n(C3CCOCC3)c(=O)n2C)cn1] |
| | (R)-8-(6-(1-(2-(6-azaspiro[2.5]octan-6-yl)ethoxy)ethyl)pyridin-3-yl)-7-fluoro- |
| | 3-methyl-1-(tetrahydro-2H-pyran-4-yl)-1H-imidazo[4,5-c]cinnolin- |
| | 2(3H)-one |
| 155 | 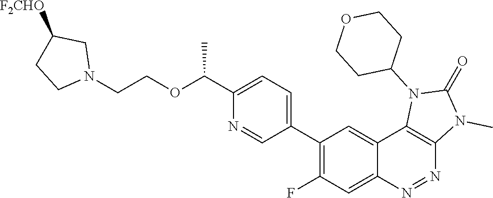 |
| | 8-(6-((R)-1-(2-((R)-3-(difluoromethoxy)pyrrolidin-1-yl)ethoxy)ethyl)- |
| | pyridin-3-yl)-7-fluoro-3-methyl-1-(tetrahydro-2H-pyran-4-yl)-1H- |
| | imidazo[4,5-c]cinnolin-2(3H)-one |
| 156 | 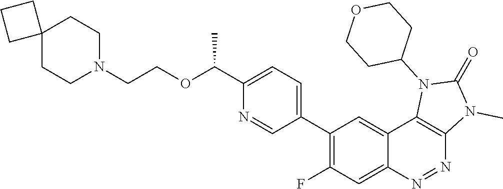 [SMILES: C[C@@H](OCCN1CCC2(CCC2)CC1)c1ccc(-c2cc3c(cc2F)nnc2c3n(C3CCOCC3)c(=O)n2C)cn1] |
| | (R)-8-(6-(1-(2-(7-azaspiro[3.5]nonan-7-yl)ethoxy)ethyl)pyridin-3-yl)-7-fluoro- |
| | 3-methyl-1-(tetrahydro-2H-pyran-4-yl)-1H-imidazo[4,5-c]cinnolin- |
| | 2(3H)-one |
| 157 | 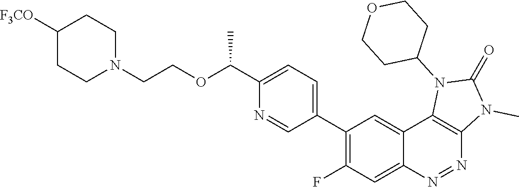 [SMILES: C[C@@H](OCCN1CCC(OC(F)(F)F)CC1)c1ccc(-c2cc3c(cc2F)nnc2c3n(C3CCOCC3)c(=O)n2C)cn1] |
| | (R)-7-fluoro-3-methyl-1-(tetrahydro-2H-pyran-4-yl)-8-(6-(1-(2-(4-(trifluoro- |
| | methoxy)piperidin-1-yl)ethoxy)ethyl)pyridin-3-yl)-1H-imidazo[4,5- |
| | c]cinnolin-2(3H)-one |
| 158 | 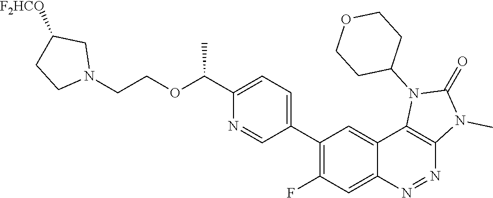 [SMILES: C[C@@H](OCCN1CC[C@H](OC(F)F)C1)c1ccc(-c2cc3c(cc2F)nnc2c3n(C3CCOCC3)c(=O)n2C)cn1] |
| | 8-(6-((R)-1-(2-((S)-3-(difluoromethoxy)pyrrolidin-1-yl)ethoxy)ethyl)- |
| | pyridin-3-yl)-7-fluoro-3-methyl-1-(tetrahydro-2H-pyran-4-yl)- |
| | 1,3-dihydro-2H-imidazo[4,5-c]cinnolin-2-one |
| 159 | 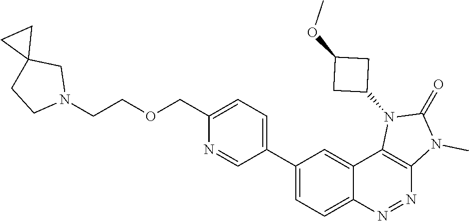 |
| | 8-(6-((2-(5-azaspiro[2.4]heptan-5-yl)ethoxy)methyl)pyridin-3-yl)-1- |
| | (trans-3-methoxycyclobutyl)-3-methyl-1H-imidazo[4,5-c]cinnolin- |
| | 2(3H)-one |
| 160 | 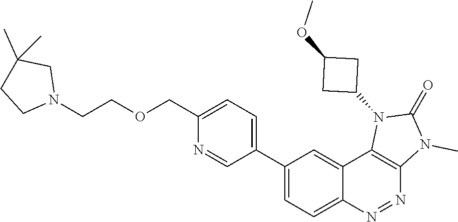 |
| | 8-(6-((2-(3,3-dimethylpyrrolidin-1-yl)ethoxy)methyl)pyridin-3-yl)-1- |
| | (trans-3-methoxycyclobutyl)-3-methyl-1H-imidazo[4,5-c]cinnolin- |
| | 2(3H)-one |
| 161 | 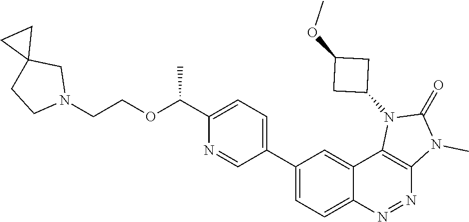 [SMILES: CO[C@H]1C[C@H](n2c(=O)n(C)c3nnc4ccc(-c5ccc([C@@H](C)OCCN6CCC7(CC7)C6)nc5)cc4c32)C1] |
| | 8-(6-((R)-1-(2-(5-azaspiro[2.4]heptan-5-yl)ethoxy)ethyl)pyridin-3-yl)-1- |
| | (trans-3-methoxycyclobutyl)-3-methyl-1H-imidazo[4,5-c]cinnolin- |
| | 2(3H)-one |
| 162 | 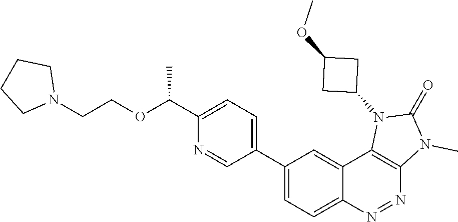 [SMILES: CO[C@H]1C[C@H](n2c(=O)n(C)c3nnc4ccc(-c5ccc([C@@H](C)OCCN6CCCC6)nc5)cc4c32)C1] |
| | 1-(trans-3-methoxycyclobutyl)-3-methyl-8-(6-((R)-1-(2-(pyrrolidin-1- |
| | yl)ethoxy)ethyl)pyridin-3-yl)-1,3-dihydro-2H-imidazo[4,5-c]cinnolin- |
| | 2-one |
| 163 | 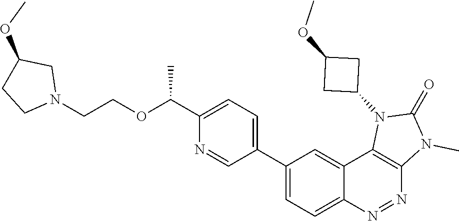 [SMILES: CO[C@@H]1CCN(CCO[C@H](C)c2ccc(-c3ccc4nnc5c(c4c3)n([C@H]3C[C@H](OC)C3)c(=O)n5C)cn2)C1] |
| | 1-(trans 3-methoxycyclobutyl)-8-(6-((R)-1-(2-((R)-3-methoxypyrrolidin- |
| | 1-yl)ethoxy)ethyl)pyridin-3-yl)-3-methyl-1H-imidazo[4,5-c]cinnolin- |
| | 2(3H)-one |
| 164 | 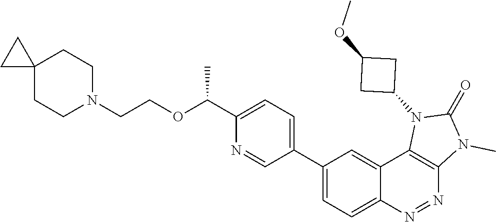 [SMILES: CO[C@H]1C[C@H](n2c(=O)n(C)c3nnc4ccc(-c5ccc([C@@H](C)OCCN6CCC7(CC6)CC7)nc5)cc4c32)C1] |
| | 8-(6-((R)-1-(2-(6-azaspiro[2.5]octan-6-yl)ethoxy)ethyl)pyridin-3-yl)-1- |
| | (trans-3-methoxycyclobutyl)-3-methyl-1H-imidazo[4,5-c]cinnolin- |
| | 2(3H)-one |
| 165 | 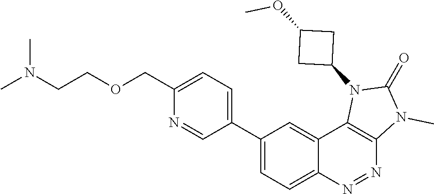 |
| | 8-(6-((2-(dimethylamino)ethoxy)methyl)pyridin-3-yl)-1-(trans-3- |
| | methoxycyclobutyl)-3-methyl-1H-imidazo[4,5-c]cinnolin-2(3H)-one |
| 166 | 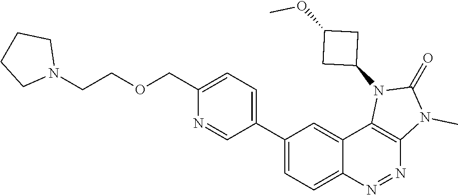 [SMILES: CO[C@H]1C[C@H](n2c(=O)n(C)c3nnc4ccc(-c5ccc(COCCN6CCCC6)nc5)cc4c32)C1] |
| | 1-(trans-3-methoxycyclobutyl)-3-methyl-8-(6-((2-(pyrrolidin-1-yl)- |
| | ethoxy)methyl)pyridin-3-yl)-1H-imidazo[4,5-c]cinnolin-2(3H)-one |
| 167 | 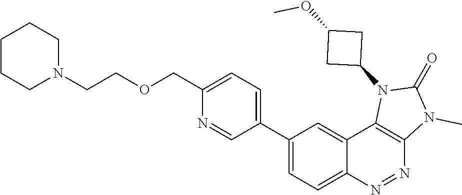 [SMILES: CO[C@H]1C[C@H](n2c(=O)n(C)c3nnc4ccc(-c5ccc(COCCN6CCCCC6)nc5)cc4c32)C1] |
| | 1-(trans-3-methoxycyclobutyl)-3-methyl-8-(6-((2-(piperidin-1-yl)- |
| | ethoxy)methyl)pyridin-3-yl)-1H-imidazo[4,5-c]cinnolin-2(3H)-one |
| 168 | 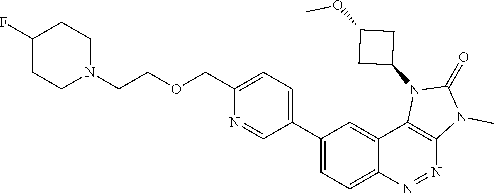 [SMILES: CO[C@H]1C[C@H](n2c(=O)n(C)c3nnc4ccc(-c5ccc(COCCN6CCC(F)CC6)nc5)cc4c32)C1] |
| | 8-(6-((2-(4-fluoropiperidin-1-yl)ethoxy)methyl)pyridin-3-yl)-1-(trans-3- |
| | methoxycyclobutyl)-3-methyl-1H-imidazo[4,5-c]cinnolin-2(3H)-one |
| 169 | 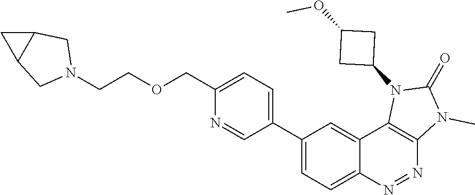 [SMILES: CO[C@H]1C[C@H](n2c(=O)n(C)c3nnc4ccc(-c5ccc(COCCN6CC7CC7C6)nc5)cc4c32)C1] |
| | 8-(6-((2-(3-azabicyclo[3.1.0]hexan-3-yl)ethoxy)methyl)pyridin-3-yl)-1- |
| | (trans-3-methoxycyclobutyl)-3-methyl-1H-imidazo[4,5-c]cinnolin- |
| | 2(3H)-one |
| 170 | 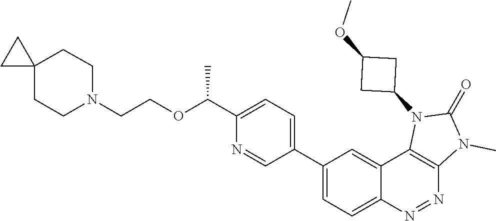 [SMILES: CO[C@H]1C[C@@H](n2c(=O)n(C)c3nnc4ccc(-c5ccc([C@@H](C)OCCN6CCC7(CC6)CC7)nc5)cc4c32)C1] |
| | 8-(6-((R)-1-(2-(6-azaspiro[2.5]octan-6-yl)ethoxy)ethyl)pyridin-3-yl)-1- |
| | ((cis)-3-methoxycyclobutyl)-3-methyl-1H-imidazo[4,5-c]cinnolin- |
| | 2(3H)-one |
| 171 | 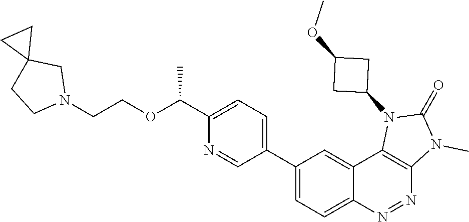 [SMILES: CO[C@H]1C[C@@H](n2c(=O)n(C)c3nnc4ccc(-c5ccc([C@@H](C)OCCN6CCC7(CC7)C6)nc5)cc4c32)C1] |
| | 8-(6-((R)-1-(2-(5-azaspiro[2.4]heptan-5-yl)ethoxy)ethyl)pyridin-3-yl)-1-(cis- |
| | 3-methoxycyclobutyl)-3-methyl-1H-imidazo[4,5-c]cinnolin-2(3H)-one |
| 172 | 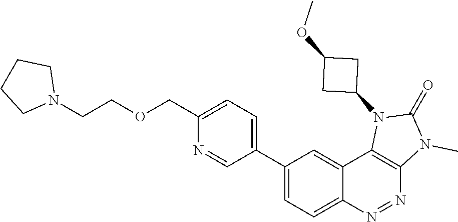 [SMILES: CO[C@H]1C[C@@H](n2c(=O)n(C)c3nnc4ccc(-c5ccc(COCCN6CCCC6)nc5)cc4c32)C1] |
| | 1-(cis-3-methoxycyclobutyl)-3-methyl-8-(6-((2-(pyrrolidin-1-yl)- |
| | ethoxy)methyl)pyridin-3-yl)-1H-imidazo[4,5-c]cinnolin-2(3H)-one |
| 173 | 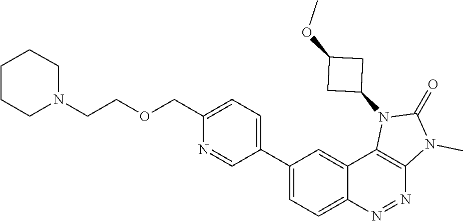 [SMILES: CO[C@H]1C[C@@H](n2c(=O)n(C)c3nnc4ccc(-c5ccc(COCCN6CCCCC6)nc5)cc4c32)C1] |
| | 1-(cis-3-methoxycyclobutyl)-3-methyl-8-(6-((2-(piperidin-1-yl)- |
| | ethoxy)methyl)pyridin-3-yl)-1H-imidazo[4,5-c]cinnolin-2(3H)-one |
| 174 | 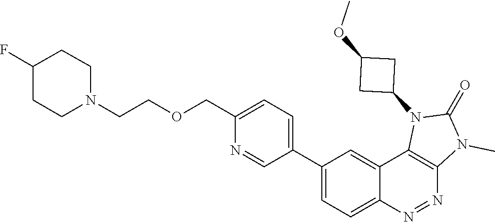 [SMILES: CO[C@H]1C[C@@H](n2c(=O)n(C)c3nnc4ccc(-c5ccc(COCCN6CCC(F)CC6)nc5)cc4c32)C1] |
| | 8-(6-((2-(4-fluoropiperidin-1-yl)ethoxy)methyl)pyridin-3-yl)-1-(cis-3- |
| | methoxycyclobutyl)-3-methyl-1H-imidazo[4,5-c]cinnolin-2(3H)-one |
| 175 | 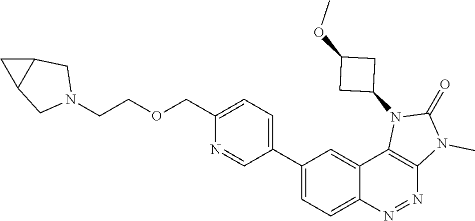 [SMILES: CO[C@H]1C[C@@H](n2c(=O)n(C)c3nnc4ccc(-c5ccc(COCCN6CC7CC7C6)nc5)cc4c32)C1] |
| | 8-(6-((2-(3-azabicyclo[3.1.0]hexan-3-yl)ethoxy)methyl)pyridin-3-yl)-1-(cis- |
| | 3-methoxycyclobutyl)-3-methyl-1H-imidazo[4,5-c]cinnolin-2(3H)-one |
| 176 | 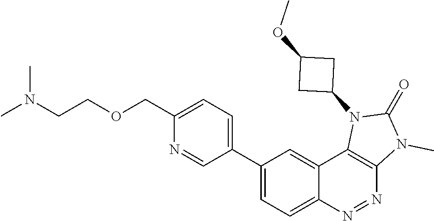 |
| | 8-(6-((2-(dimethylamino)ethoxy)methyl)pyridin-3-yl)-1-(cis-3-meth- |
| | oxycyclobutyl)-3-methyl-1H-imidazo[4,5-c]cinnolin-2(3H)-one |
| 177 | 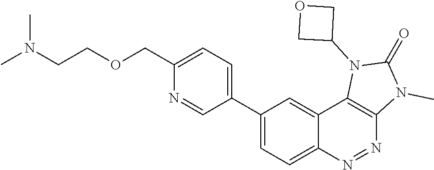 |
| | 8-(6-((2-(dimethylamino)ethoxy)methyl)pyridin-3-yl)-3-methyl-1- |
| | (oxetan-3-yl)-1H-imidazo[4,5-c]cinnolin-2(3H)-one |
| 178 | 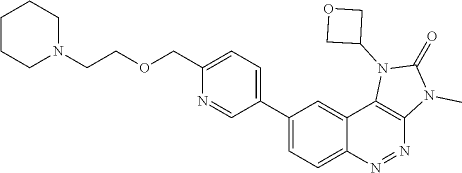 [SMILES: Cn1c(=O)n(C2COC2)c2c3cc(-c4ccc(COCCN5CCCCC5)nc4)ccc3nnc21] |
| | 3-methyl-1-(oxetan-3-yl)-8-(6-((2-(piperidin-1-yl)ethoxy)methyl)- |
| | pyridin-3-yl)-1H-imidazo[4,5-c]cinnolin-2(3H)-one |
| 179 | 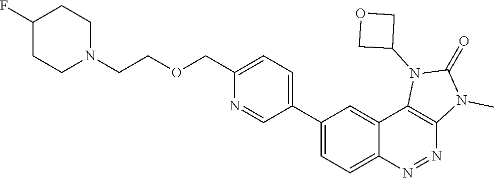 [SMILES: Cn1c(=O)n(C2COC2)c2c3cc(-c4ccc(COCCN5CCC(F)CC5)nc4)ccc3nnc21] |
| | 8-(6-((2-(4-fluoropiperidin-1-yl)ethoxy)methyl)pyridin-3-yl)-3-methyl- |
| | 1-(oxetan-3-yl)-1H-imidazo[4,5-c]cinnolin-2(3H)-one |
| 180 | 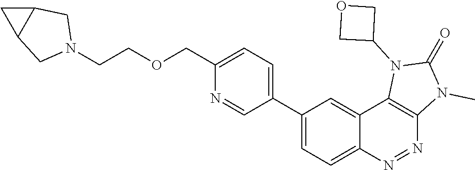 [SMILES: Cn1c(=O)n(C2COC2)c2c3cc(-c4ccc(COCCN5CC6CC6C5)nc4)ccc3nnc21] |
| | 8-(6-((2-(4-fluoropiperidin-1-yl)ethoxy)methyl)pyridin-3-yl)-1-((1s,3s)-3- |
| | methoxycyclobutyl)-3-methyl-1H-imidazo[4,5-c]cinnolin-2(3H)-one |
| 181 | 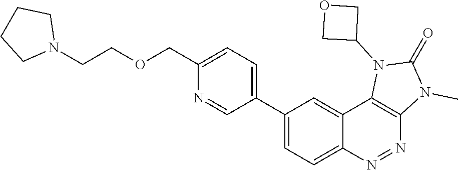 [SMILES: Cn1c(=O)n(C2COC2)c2c3cc(-c4ccc(COCCN5CCCC5)nc4)ccc3nnc21] |
| | 3-methyl-1-(oxetan-3-yl)-8-(6-((2-(pyrrolidin-1-yl)ethoxy)methyl)pyridin- |
| | 3-yl)-1H-imidazo[4,5-c]cinnolin-2(3H)-one |
| 182 | 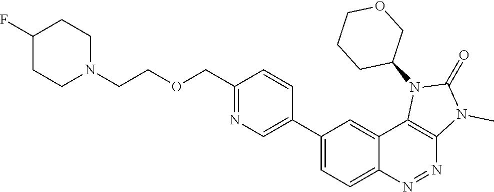 [SMILES: Cn1c(=O)n([C@H]2CCCOC2)c2c3cc(-c4ccc(COCCN5CCC(F)CC5)nc4)ccc3nnc21] |
| | (S)-8-(6-((2-(4-fluoropiperidin-1-yl)ethoxy)methyl)pyridin-3-yl)-3-methyl- |
| | 1-(tetrahydro-2H-pyran-3-yl)-1H-imidazo[4,5-c]cinnolin-2(3H)-one |
| 183 | 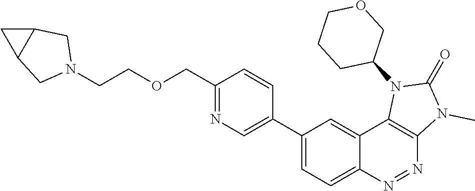 [SMILES: Cn1c(=O)n([C@H]2CCCOC2)c2c3cc(-c4ccc(COCCN5CC6CC6C5)nc4)ccc3nnc21] |
| | 8-(6-((2-(3-azabicyclo[3.1.0]hexan-3-yl)ethoxy)methyl)pyridin-3-yl)-3- |
| | methyl-1-((S)-tetrahydro-2H-pyran-3-yl)-1H-imidazo[4,5-c]cinnolin- |
| | 2(3H)-one |
| 184 | 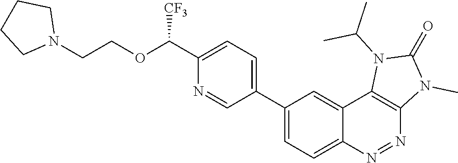 [SMILES: CC(C)n1c(=O)n(C)c2nnc3ccc(-c4ccc([C@H](OCCN5CCCC5)C(F)(F)F)nc4)cc3c21] |
| | (S)-1-isopropyl-3-methyl-8-(6-(2,2,2-trifluoro-1-(2-(pyrrolidin-1-yl)ethoxy)- |
| | ethyl)pyridin-3-yl)-1,3-dihydro-2H-imidazo[4,5-c]cinnolin-2-one |
| 185 | 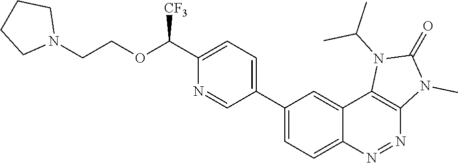 [SMILES: CC(C)n1c(=O)n(C)c2nnc3ccc(-c4ccc([C@@H](OCCN5CCCC5)C(F)(F)F)nc4)cc3c21] |
| | (R)-1-isopropyl-3-methyl-8-(6-(2,2,2-trifluoro-1-(2-(pyrrolidin-1-yl)ethoxy)- |
| | ethyl)pyridin-3-yl)-1,3-dihydro-2H-imidazo[4,5-c]cinnolin-2-one |
| 186 | 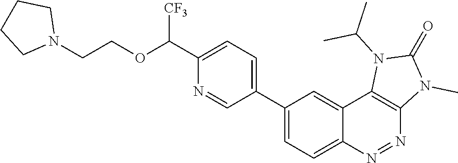 [SMILES: CC(C)n1c(=O)n(C)c2nnc3ccc(-c4ccc(C(OCCN5CCCC5)C(F)(F)F)nc4)cc3c21] |
| | 1-isopropyl-3-methyl-8-(6-(2,2,2-trifluoro-1-(2-(pyrrolidin-1-yl)ethoxy)- |
| | ethyl)pyridin-3-yl)-1,3-dihydro-2H-imidazo[4,5-c]cinnolin-2-one |
| 187 | 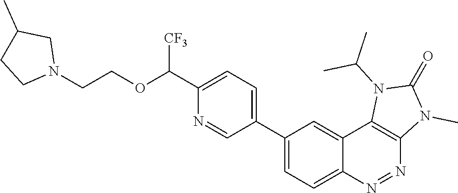 [SMILES: CC1CCN(CCOC(c2ccc(-c3ccc4nnc5c(c4c3)n(C(C)C)c(=O)n5C)cn2)C(F)(F)F)C1] |
| | 1-isopropyl-3-methyl-8-(6-(2,2,2-trifluoro-1-(2-(3-methylpyrrolidin-1-yl) |
| | ethoxy)ethyl)pyridin-3-yl)-1,3-dihydro-2H-imidazo[4,5-c]cinnolin-2-one |
| 188 | 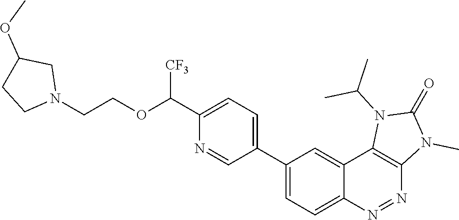 [SMILES: COC1CCN(CCOC(c2ccc(-c3ccc4nnc5c(c4c3)n(C(C)C)c(=O)n5C)cn2)C(F)(F)F)C1] |
| | 1-isopropyl-3-methyl-8-(6-(2,2,2-trifluoro-1-(2-(3-methoxypyrrolidin-1-yl)- |
| | ethoxy)ethyl)pyridin-3-yl)-1,3-dihydro-2H-imidazo[4,5-c]cinnolin-2-one |
| 189 | 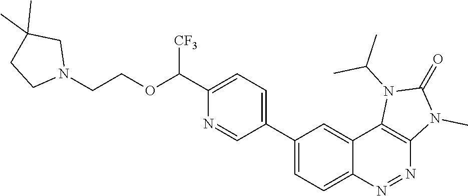 |
| | 8-(6-(1-(2-(3,3-dimethylpyrrolidin-1-yl)ethoxy)-2,2,2-trifluoroethyl)- |
| | pyridin-3-yl)-1-isopropyl-3-methyl-1,3-dihydro-2H-imidazo[4,5-c]- |
| | cinnolin-2-one |
| 190 | 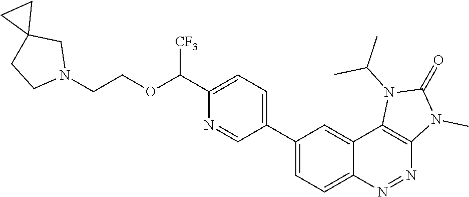 [SMILES: CC(C)n1c(=O)n(C)c2nnc3ccc(-c4ccc(C(OCCN5CCC6(CC6)C5)C(F)(F)F)nc4)cc3c21] |
| | 8-(6-(1-(2-(5-azaspiro[2.4]heptan-5-yl)ethoxy)-2,2,2-trifluoroethyl)- |
| | pyridin-3-yl)-1-isopropyl-3-methyl-1,3-dihydro-2H-imidazo[4,5- |
| | c]cinnolin-2-one |
| 191 | 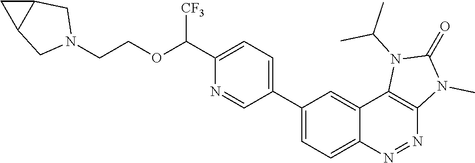 [SMILES: CC(C)n1c(=O)n(C)c2nnc3ccc(-c4ccc(C(OCCN5CC6CC6C5)C(F)(F)F)nc4)cc3c21] |
| | 8-(6-(1-(2-(3-azabicyclo[3.1.0]hexan-3-yl)ethoxy)-2,2,2-trifluoroethyl)- |
| | pyridin-3-yl)-1-isopropyl-3-methyl-1,3-dihydro-2H-imidazo[4,5- |
| | c]cinnolin-2-one |
| 192 | 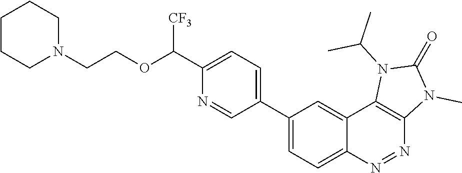 [SMILES: CC(C)n1c(=O)n(C)c2nnc3ccc(-c4ccc(C(OCCN5CCCCC5)C(F)(F)F)nc4)cc3c21] |
| | 1-isopropyl-3-methyl-8-(6-(2,2,2-trifluoro-1-(2-(piperidin-1-yl)ethoxy)- |
| | ethyl)pyridin-3-yl)-1,3-dihydro-2H-imidazo[4,5-c]cinnolin-2-one |
| 193 | 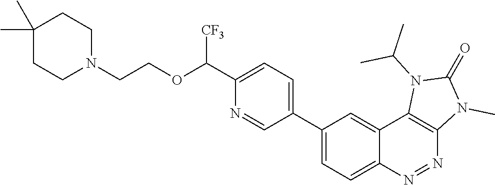 |
| | 8-(6-(1-(2-(4,4-dimethylpiperidin-1-yl)ethoxy)-2,2,2-trifluoroethyl)- |
| | pyridin-3-yl)-1-isopropyl-3-methyl-1,3-dihydro-2H-imidazo- |
| | [4,5-c]cinnolin-2-one |
| 194 | 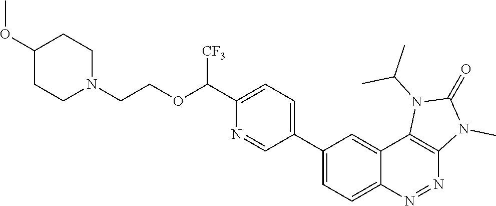 [SMILES: COC1CCN(CCOC(c2ccc(-c3ccc4nnc5c(c4c3)n(C(C)C)c(=O)n5C)cn2)C(F)(F)F)CC1] |
| | 1-isopropyl-3-methyl-8-(6-(2,2,2-trifluoro-1-(2-(4-methoxypiperidin-1-yl)- |
| | ethoxy)ethyl)pyridin-3-yl)-1,3-dihydro-2H-imidazo[4,5-c]cinnolin- |
| | 2-one |
| 195 | 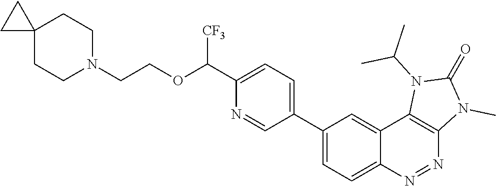 [SMILES: CC(C)n1c(=O)n(C)c2nnc3ccc(-c4ccc(C(OCCN5CCC6(CC5)CC6)C(F)(F)F)nc4)cc3c21] |
| | 8-(6-(1-(2-(6-azaspiro[2.5]octan-6-yl)ethoxy)-2,2,2-trifluoroethyl)pyridin- |
| | 3-yl)-1-isopropyl-3-methyl-1,3-dihydro-2H-imidazo[4,5-c]cinnolin- |
| | 2-one |
| 196 | 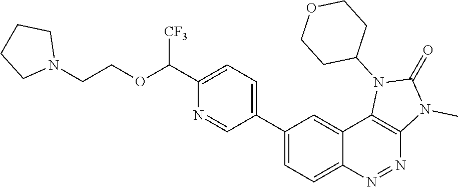 [SMILES: Cn1c(=O)n(C2CCOCC2)c2c3cc(-c4ccc(C(OCCN5CCCC5)C(F)(F)F)nc4)ccc3nnc21] |
| | 3-methyl-1-(tetrahydro-2H-pyran-4-yl)-8-(6-(2,2,2-trifluoro-1-(2- |
| | (pyrrolidin-1-yl)ethoxy)ethyl)pyridin-3-yl)-1,3-dihydro-2H- |
| | imidazo[4,5-c]cinnolin-2-one |
| 197 | 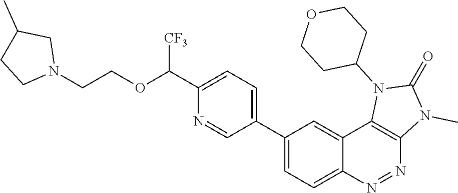 [SMILES: CC1CCN(CCOC(c2ccc(-c3ccc4nnc5c(c4c3)n(C3CCOCC3)c(=O)n5C)cn2)C(F)(F)F)C1] |
| | 3-methyl-1-(tetrahydro-2H-pyran-4-yl)-8-(6-(2,2,2-trifluoro-1-(2-(3- |
| | methylpyrrolidin-1-yl)ethoxy)ethyl)pyridin-3-yl)-1,3-dihydro-2H- |
| | imidazo[4,5-c]cinnolin-2-one |
| 198 | 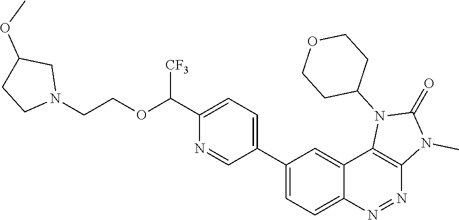 [SMILES: COC1CCN(CCOC(c2ccc(-c3ccc4nnc5c(c4c3)n(C3CCOCC3)c(=O)n5C)cn2)C(F)(F)F)C1] |
| | 3-methyl-1-(tetrahydro-2H-pyran-4-yl)-8-(6-(2,2,2-trifluoro-1-(2-(3- |
| | methoxypyrrolidin-1-yl)ethoxy)ethyl)pyridin-3-yl)-1,3-dihydro-2H- |
| | imidazo[4,5-c]cinnolin-2-one |
| 199 | 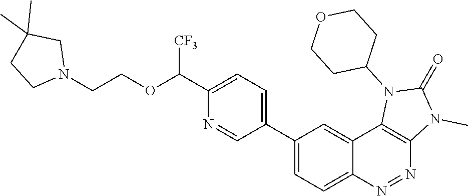 |
| | 8-(6-(1-(2-(3,3-dimethylpyrrolidin-1-yl)ethoxy)-2,2,2-trifluoroethyl)- |
| | pyridin-3-yl)-3-methyl-1-(tetrahydro-2H-pyran-4-yl)-1,3-dihydro- |
| | 2H-imidazo[4,5-c]cinnolin-2-one |
| 200 | 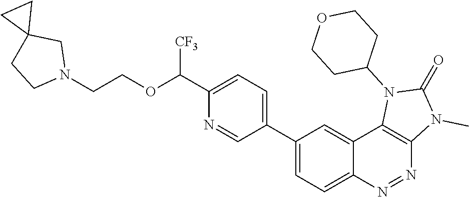 [SMILES: Cn1c(=O)n(C2CCOCC2)c2c3cc(-c4ccc(C(OCCN5CCC6(CC6)C5)C(F)(F)F)nc4)ccc3nnc21] |
| | 8-(6-(1-(2-(5-azaspiro[2.4]heptan-5-yl)ethoxy)-2,2,2-trifluoroethyl)- |
| | pyridin-3-yl)-3-methyl-1-(tetrahydro-2H-pyran-4-yl)-1,3-dihydro- |
| | 2H-imidazo[4,5-c]cinnolin-2-one |
| 201 | 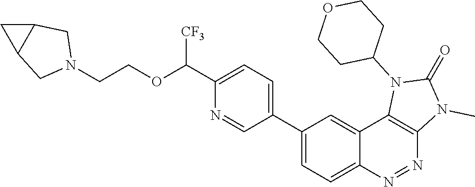 [SMILES: Cn1c(=O)n(C2CCOCC2)c2c3cc(-c4ccc(C(OCCN5CC6CC6C5)C(F)(F)F)nc4)ccc3nnc21] |
| | 8-(6-(1-(2-(3-azabicyclo[3.1.0]hexan-3-yl)ethoxy)-2,2,2-trifluoroethyl)- |
| | pyridin-3-yl)-3-methyl-1-(tetrahydro-2H-pyran-4-yl)-1,3-dihydro- |
| | 2H-imidazo[4,5-c]cinnolin-2-one |
| 202 | 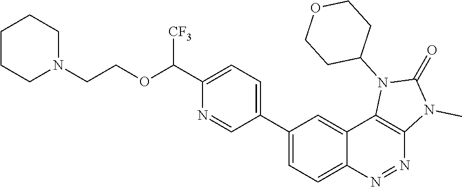 [SMILES: Cn1c(=O)n(C2CCOCC2)c2c3cc(-c4ccc(C(OCCN5CCCCC5)C(F)(F)F)nc4)ccc3nnc21] |
| | 3-methyl-1-(tetrahydro-2H-pyran-4-yl)-8-(6-(2,2,2-trifluoro-1-(2- |
| | (piperidin-1-yl)ethoxy)ethyl)pyridin-3-yl)-1,3-dihydro-2H-imidazo- |
| | [4,5-c]cinnolin-2-one |
| 203 | 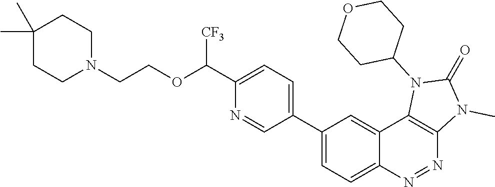 |
| | 8-(6-(1-(2-(4,4-dimethylpiperidin-1-yl)ethoxy)-2,2,2-trifluoroethyl)- |
| | pyridin-3-yl)-3-methyl-1-(tetrahydro-2H-pyran-4-yl)-1,3-dihydro- |
| | 2H-imidazo[4,5-c]cinnolin-2-one |
| 204 | 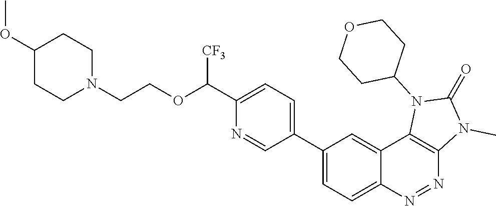 [SMILES: COC1CCN(CCOC(c2ccc(-c3ccc4nnc5c(c4c3)n(C3CCOCC3)c(=O)n5C)cn2)C(F)(F)F)CC1] |
| | 3-methyl-1-(tetrahydro-2H-pyran-4-yl)-8-(6-(2,2,2-trifluoro-1-(2-(4-methoxy- |
| | piperidin-1-yl)ethoxy)ethyl)pyridin-3-yl)-1,3-dihydro-2H-imidazo[4,5- |
| | c]cinnolin-2-one |
| 205 | 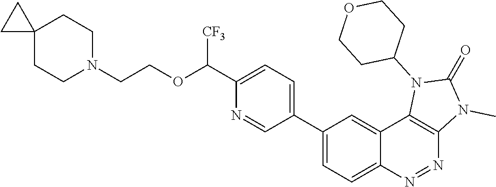 [SMILES: Cn1c(=O)n(C2CCOCC2)c2c3cc(-c4ccc(C(OCCN5CCC6(CC5)CC6)C(F)(F)F)nc4)ccc3nnc21] |
| | 8-(6-(1-(2-(6-azaspiro[2.5]octan-6-yl)ethoxy)-2,2,2-trifluoroethyl)pyridin- |
| | 3-yl)-3-methyl-1-(tetrahydro-2H-pyran-4-yl)-1,3-dihydro-2H-imidazo- |
| | [4,5-c]cinnolin-2-one |
| 206 | 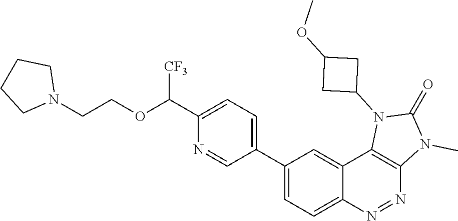 [SMILES: COC1CC(n2c(=O)n(C)c3nnc4ccc(-c5ccc(C(OCCN6CCCC6)C(F)(F)F)nc5)cc4c32)C1] |
| | 1-(3-methoxycyclobutyl)-3-methyl-8-(6-(2,2,2-trifluoro-1-(2-(pyrrolidin- |
| | 1-yl)ethoxy)ethyl)pyridin-3-yl)-1,3-dihydro-2H-imidazo[4,5-c]- |
| | cinnolin-2-one |
| 207 | 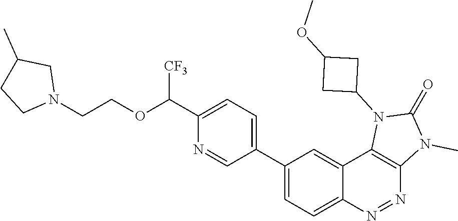 [SMILES: COC1CC(n2c(=O)n(C)c3nnc4ccc(-c5ccc(C(OCCN6CCC(C)C6)C(F)(F)F)nc5)cc4c32)C1] |
| | 1-(3-methoxycyclobutyl)-3-methyl-8-(6-(2,2,2-trifluoro-1-(2-(3-methyl- |
| | pyrrolidin-1-yl)ethoxy)ethyl)pyridin-3-yl)-1,3-dihydro-2H-imidazo- |
| | [4,5-c]cinnolin-2-one |
| 208 | 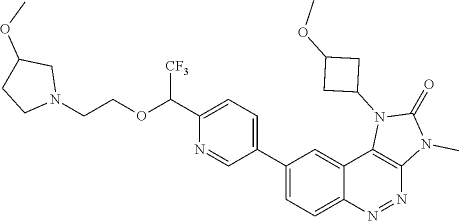 [SMILES: COC1CC(n2c(=O)n(C)c3nnc4ccc(-c5ccc(C(OCCN6CCC(OC)C6)C(F)(F)F)nc5)cc4c32)C1] |
| | 1-(3-methoxycyclobutyl)-3-methyl-8-(6-(2,2,2-trifluoro-1-(2-(3-methoxy- |
| | pyrrolidin-1-yl)ethoxy)ethyl)pyridin-3-yl)-1,3-dihydro-2H-imidazo- |
| | [4,5-c]cinnolin-2-one |
| 209 | 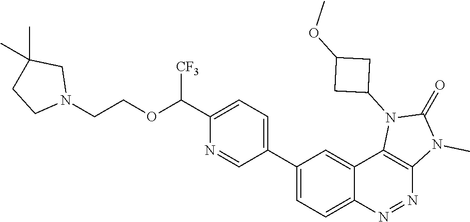 |
| | 8-(6-(1-(2-(3,3-dimethylpyrrolidin-1-yl)ethoxy)-2,2,2-trifluoroethyl)- |
| | pyridin-3-yl)-1-(3-methoxycyclobutyl)-3-methyl-1,3-dihydro-2H- |
| | imidazo[4,5-c]cinnolin-2-one |
| 210 | 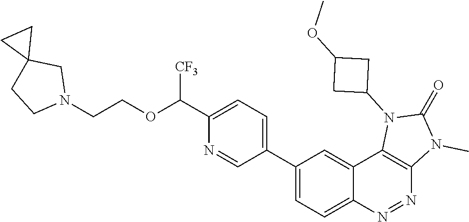 [SMILES: COC1CC(n2c(=O)n(C)c3nnc4ccc(-c5ccc(C(OCCN6CCC7(CC7)C6)C(F)(F)F)nc5)cc4c32)C1] |
| | 8-(6-(1-(2-(5-azaspiro[2.4]heptan-5-yl)ethoxy)-2,2,2-trifluoroethyl)- |
| | pyridin-3-yl)-1-(3-methoxycyclobutyl)-3-methyl-1,3-dihydro-2H- |
| | imidazo[4,5-c]cinnolin-2-one |
| 211 | 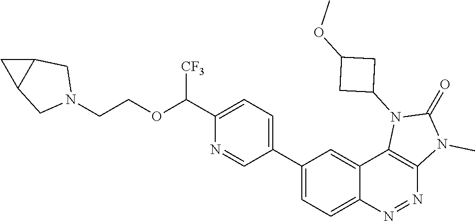 [SMILES: COC1CC(n2c(=O)n(C)c3nnc4ccc(-c5ccc(C(OCCN6CC7CC7C6)C(F)(F)F)nc5)cc4c32)C1] |
| | 8-(6-(1-(2-(3-azabicyclo[3.1.0]hexan-3-yl)ethoxy)-2,2,2-trifluoroethyl)- |
| | pyridin-3-yl)-1-(3-methoxycyclobutyl)-3-methyl-1,3-dihydro-2H- |
| | imidazo[4,5-c]cinnolin-2-one |
| 212 | 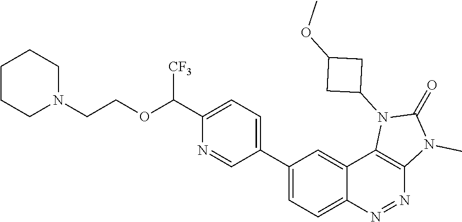 [SMILES: COC1CC(n2c(=O)n(C)c3nnc4ccc(-c5ccc(C(OCCN6CCCCC6)C(F)(F)F)nc5)cc4c32)C1] |
| | 1-(3-methoxycyclobutyl)-3-methyl-8-(6-(2,2,2-trifluoro-1-(2-(piperidin- |
| | 1-yl)ethoxy)ethyl)pyridin-3-yl)-1,3-dihydro-2H-imidazo[4,5-c]cinnolin- |
| | 2-one |
| 213 | 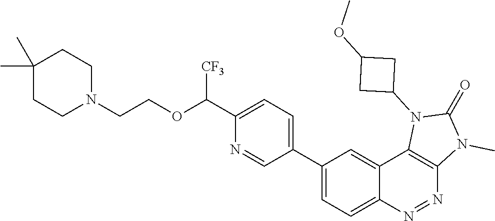 |
| | 8-(6-(1-(2-(4,4-dimethylpiperidin-1-yl)ethoxy)-2,2,2-trifluoroethyl)pyridin- |
| | 3-yl)-1-(3-methoxycyclobutyl)-3-methyl-1,3-dihydro-2H-imidazo[4,5- |
| | c]cinnolin-2-one |
| 214 | 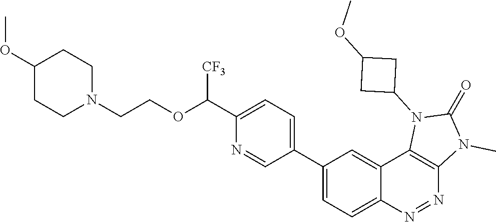 [SMILES: COC1CCN(CCOC(c2ccc(-c3ccc4nnc5c(c4c3)n(C3CC(OC)C3)c(=O)n5C)cn2)C(F)(F)F)CC1] |
| | 1-(3-methoxycyclobutyl)-3-methyl-8-(6-(2,2,2-trifluoro-1-(2-(4-methoxy- |
| | piperidin-1-yl)ethoxy)ethyl)pyridin-3-yl)-1,3-dihydro-2H-imidazo[4,5- |
| | c]cinnolin-2-one |
| 215 | 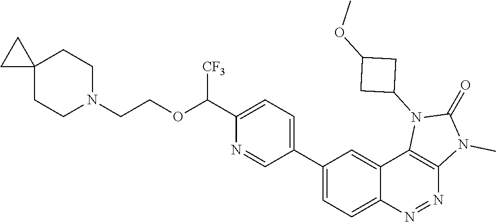 [SMILES: COC1CC(n2c(=O)n(C)c3nnc4ccc(-c5ccc(C(OCCN6CCC7(CC6)CC7)C(F)(F)F)nc5)cc4c32)C1] |
| | 8-(6-(1-(2-(6-azaspiro[2.5]octan-6-yl)ethoxy)-2,2,2-trifluoroethyl)pyridin- |
| | 3-yl)-1-(3-methoxycyclobutyl)-3-methyl-1,3-dihydro-2H-imidazo[4,5- |
| | c]cinnolin-2-one |
| 216 | 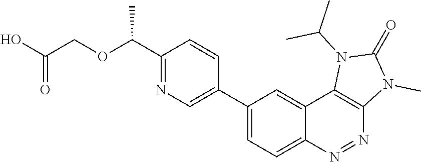 [SMILES: CC(C)n1c(=O)n(C)c2nnc3ccc(-c4ccc([C@@H](C)OCC(=O)O)nc4)cc3c21] |
| | (R)-2-(1-(5-(1-isopropyl-3-methyl-2-oxo-2,3-dihydro-1H-imidazo- |
| | [4,5-c]cinnolin-8-yl)pyridin-2-yl)ethoxy)acetic acid |
| 217 | 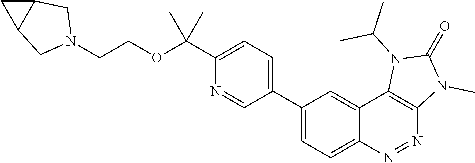 [SMILES: CC(C)n1c(=O)n(C)c2nnc3ccc(-c4ccc(C(C)(C)OCCN5CC6CC6C5)nc4)cc3c21] |
| | 8-(6-(2-(2-(3-azabicyclo[3.1.0]hexan-3-yl)ethoxy)propan-2-yl)pyridin-3- |
| | yl)-1-isopropyl-3-methyl-1H-imidazo[4,5-c]cinnolin-2(3H)-one |
| 218 | 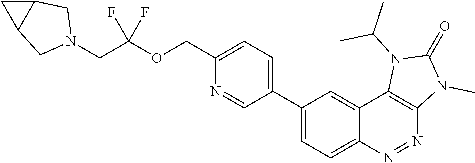 [SMILES: CC(C)n1c(=O)n(C)c2nnc3ccc(-c4ccc(COC(F)(F)CN5CC6CC6C5)nc4)cc3c21] |
| | 8-(6-((2-(3-azabicyclo[3.1.0]hexan-3-yl)-1,1-difluoroethoxy)methyl)- |
| | pyridin-3-yl)-1-isopropyl-3-methyl-1H-imidazo[4,5-c]cinnolin- |
| | 2(3H)-one |
| 219 | 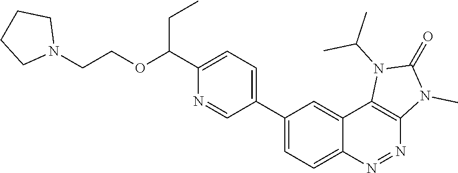 [SMILES: CCC(OCCN1CCCC1)c1ccc(-c2ccc3nnc4c(c3c2)n(C(C)C)c(=O)n4C)cn1] |
| | 1-isopropyl-3-methyl-8-(6-(1-(2-(pyrrolidin-1-yl)ethoxy)propyl)pyridin- |
| | 3-yl)-1H-imidazo[4,5-c]cinnolin-2(3H)-one |
| 220 | 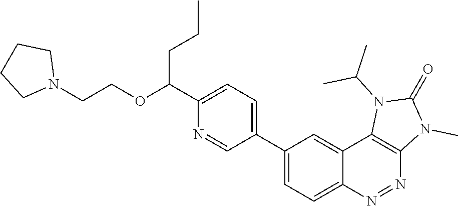 [SMILES: CCCC(OCCN1CCCC1)c1ccc(-c2ccc3nnc4c(c3c2)n(C(C)C)c(=O)n4C)cn1] |
| | 1-isopropyl-3-methyl-8-(6-(1-(2-(pyrrolidin-1-yl)ethoxy)butyl)pyridin- |
| | 3-yl)-1H-imidazo[4,5-c]cinnolin-2(3H)-one |
| 221 | 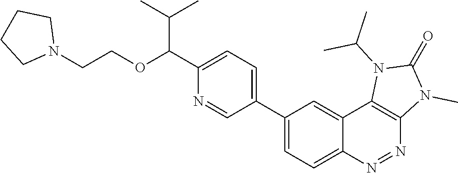 [SMILES: CC(C)C(OCCN1CCCC1)c1ccc(-c2ccc3nnc4c(c3c2)n(C(C)C)c(=O)n4C)cn1] |
| | 1-isopropyl-3-methyl-8-(6-(2-methyl-1-(2-(pyrrolidin-1-yl)ethoxy)- |
| | propyl)pyridin-3-yl)-1H-imidazo[4,5-c]cinnolin-2(3H)-one |
| 222 | 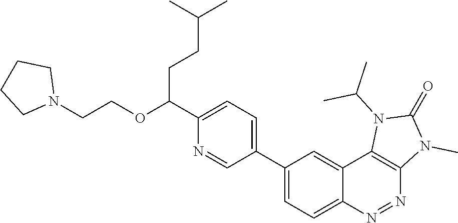 [SMILES: CC(C)CCC(OCCN1CCCC1)c1ccc(-c2ccc3nnc4c(c3c2)n(C(C)C)c(=O)n4C)cn1] |
| | 1-isopropyl-3-methyl-8-(6-(4-methyl-1-(2-(pyrrolidin-1-yl)ethoxy)- |
| | pentyl)pyridin-3-yl)-1H-imidazo[4,5-c]cinnolin-2(3H)-one |

Compounds provided herein are described with reference to both generic formulae and specific compounds. In addition, the compounds of the present disclosure may exist in a number of different forms or derivatives, including but not limited to, stereoisomers, racemic mixtures, regioisomers, tautomers, salts, prodrugs, soft drugs, active metabolic derivatives (active metabolites), solvated forms, different crystal forms or polymorphs, all within the scope of the present disclosure.

The compounds of present disclosure can comprise one or more asymmetric centers, and thus can exist in various stereoisomeric forms, e.g., enantiomers and/or diastereomers. Thus, the compounds of present disclosure and compositions thereof may be in the form of an individual enantiomer, diastereomer or geometric isomer, or may be in the form of a mixture of stereoisomers. In certain embodiments, the compounds of the present disclosure are enantiopure compounds. In certain embodiments, mixtures of enantiomers or diastereomers are provided.

The term “enantiomer” refers to two stereoisomers of a compound which are non-superimposable mirror images of one another. The term “diastereomer” refers to a pair of optical isomers which are not mirror images of one another. Diastereomers have different physical properties, e.g. melting points, boiling points, spectral properties, and reactivities.

Furthermore, certain compounds, as described herein may have one or more double bonds that can exist as either the Z or E isomer, unless otherwise indicated. The present disclosure additionally encompasses the compounds as individual isomers substantially free of other isomers and alternatively, as mixtures of various isomers, e.g., racemic mixtures of enantiomers. In addition to the above-mentioned compounds per se, this disclosure also encompasses compositions comprising one or more compounds.

As used herein, the term “isomers” includes any and all geometric isomers and stereoisomers. For example, “isomers” include cis- and trans-isomers, E- and Z-isomers, R- and S-enantiomers, diastereomers, (D)-isomers, (L)-isomers, racemic mixtures thereof, and other mixtures thereof, as falling within the scope of the invention. For instance, a stereoisomer may, in some embodiments, be provided substantially free of one or more corresponding stereoisomers, and may also be referred to as “stereochemically enriched”.

Where a particular enantiomer is preferred, it may, in some embodiments be provided substantially free of the opposite enantiomer, and may also be referred to as “optically enriched”. “Optically enriched”, as used herein, means that the compound is made up of a significantly greater proportion of one enantiomer. In certain embodiments, the compound is made up of at least about 90% by weight of a preferred enantiomer. In other embodiments, the compound is made up of at least about 95%, 98%, or 99% by weight of a preferred enantiomer. Preferred enantiomers may be isolated from racemic mixtures by any method known to those skilled in the art, including chiral high pressure liquid chromatography (HPLC) and the formation and crystallization of chiral salts or prepared by asymmetric syntheses. See, for example, Jacques, et al., Enantiomers, Racemates and Resolutions (Wiley Interscience, New York, 1981); Wilen, S. H., et al., Tetrahedron 33:2725 (1977); Eliel, E. L. Stereochemistry of Carbon Compounds (McGraw-Hill, N Y, 1962); Wilen, S. H. Tables of Resolving Agents and Optical Resolutions p. 268 (E. L. Eliel, Ed., Univ. of Notre Dame Press, Notre Dame, IN 1972).

The compounds of the present disclosure may also exist in different tautomeric forms, and all such forms are embraced within the scope of the present disclosure. The term “tautomer” or “tautomeric form” refers to structural isomers of different energies which are interconvertible via a low energy barrier. The presence and concentrations of the isomeric forms will depend on the environment the compound is found in and may be different depending upon, for example, whether the compound is a solid or is in an organic or aqueous solution. By way of examples, proton tautomers (also known as prototropic tautomers) include interconversions via migration of a proton, such as keto-enol, amide-imidic acid, lactam-lactim, imine-enamine isomerizations and annular forms where a proton can occupy two or more positions of a heterocyclic system. Valence tautomers include interconversions by reorganization of some of the bonding electrons. Tautomers can be in equilibrium or sterically locked into one form by appropriate substitution. Compounds of the present disclosure identified by name or structure as one particular tautomeric form are intended to include other tautomeric forms unless otherwise specified.

As used herein, the term “prodrugs” refers to compounds or pharmaceutically acceptable salts thereof which, when metabolized under physiological conditions or when converted by solvolysis, yield the desired active compound. Prodrugs include, without limitation, esters, amides, carbamates, carbonates, ureides, solvates, or hydrates of the active compound. Typically, the prodrug is inactive, or less active than the active compound, but may provide one or more advantageous handling, administration, and/or metabolic properties. For example, some prodrugs are esters of the active compound; during metabolysis, the ester group is cleaved to yield the active drug. Also, some prodrugs are activated enzymatically to yield the active compound, or a compound which, upon further chemical reaction, yields the active compound. Prodrugs may proceed from prodrug form to active form in a single step or may have one or more intermediate forms which may themselves have activity or may be inactive. Preparation and use of prodrugs is discussed in T. Higuchi and V. Stella, “Pro-drugs as Novel Delivery Systems”, Vol. 14 of the A.C.S. Symposium Series, in Bioreversible Carriers in Drug Design, ed. Edward B. Roche, American Pharmaceutical Association and Pergamon Press, 1987; in Prodrugs: Challenges and Rewards, ed. V. Stella, R. Borchardt, M. Hageman, R. Oliyai, H. Maag, J. Tilley, Springer-Verlag New York, 2007, all of which are hereby incorporated by reference in their entirety.

As used herein, the term “soft drug” refers to compounds that exert a pharmacological effect but break down to inactive metabolites degradants so that the activity is of limited time. See, for example, “Soft drugs: Principles and methods for the design of safe drugs”, Nicholas Bodor, Medicinal Research Reviews, Vol. 4, No. 4, 449-469, 1984, which is hereby incorporated by reference in its entirety.

As used herein, the term “metabolite”, e.g., active metabolite overlaps with prodrug as described above. Thus, such metabolites are pharmacologically active compounds or compounds that further metabolize to pharmacologically active compounds that are derivatives resulting from metabolic process in the body of a subject. For example, such metabolites may result from oxidation, reduction, hydrolysis, amidation, deamidation, esterification, deesterification, enzymatic cleavage, and the like, of the administered compound or salt or prodrug. Of these, active metabolites are such pharmacologically active derivative compounds. For prodrugs, the prodrug compound is generally inactive or of lower activity than the metabolic product. For active metabolites, the parent compound may be either an active compound or may be an inactive prodrug.

Prodrugs and active metabolites may be identified using routine techniques known in the art. See, e.g., Bertolini et al, 1997, J Med Chem 40:2011-2016; Shan et al., J Pharm Sci 86:756-757; Bagshawe, 1995, Drug Dev Res 34:220-230; Wermuth, supra.

As used herein, the term “active intermediate” refers to intermediate compound in the synthetic process, which exhibits the same or essentially the same biological activity as the final synthesized compound.

Compounds of the present disclosure can be formulated as or be in the form of pharmaceutically acceptable salts. Unless specified to the contrary, a compound provided herein includes pharmaceutically acceptable salts of such compound.

As used herein, the term “pharmaceutically acceptable” indicates that the substance or composition is compatible chemically and/or toxicologically, with the other ingredients comprising a formulation, and/or the subjects being treated therewith.

As used herein, the term “pharmaceutically acceptable salt”, unless otherwise indicated, includes salts that retain the biological effectiveness of the free acids and bases of the specified compound and that are not biologically or otherwise undesirable. Contemplated pharmaceutically acceptable salt forms include, but are not limited to, mono, bis, tris, tetrakis, and so on. Pharmaceutically acceptable salts are non-toxic in the amounts and concentrations at which they are administered. The preparation of such salts can facilitate the pharmacological use by altering the physical characteristics of a compound without preventing it from exerting its physiological effect. Useful alterations in physical properties include lowering the melting point to facilitate transmucosal administration and increasing the solubility to facilitate administering higher concentrations of the drug.

Pharmaceutically acceptable salts include acid addition salts such as those containing sulfate, chloride, hydrochloride, fumarate, maleate, phosphate, sulfamate, acetate, citrate, lactate, tartrate, methanesulfonate, ethanesulfonate, benzenesulfonate, p-toluenesulfonate, cyclohexylsulfamate and quinate. Pharmaceutically acceptable salts can be obtained from acids such as hydrochloric acid, maleic acid, sulfuric acid, phosphoric acid, sulfamic acid, acetic acid, citric acid, lactic acid, tartaric acid, malonic acid, methanesulfonic acid, ethanesulfonic acid, benzenesulfonic acid, p-toluenesulfonic acid, cyclohexylsulfamic acid, fumaric acid, and quinic acid.

Pharmaceutically acceptable salts also include basic addition salts such as those containing benzathine, chloroprocaine, choline, diethanolamine, ethanolamine, t-butylamine, ethylenediamine, meglumine, procaine, aluminum, calcium, lithium, magnesium, potassium, sodium, ammonium, alkylamine, and zinc, when acidic functional groups, such as carboxylic acid or phenol are present. For example, see Remington's Pharmaceutical Sciences, 19^{th} ed., Mack Publishing Co., Easton, PA, Vol. 2, p. 1457, 1995; “Handbook of Pharmaceutical Salts: Properties, Selection, and Use” by Stahl and Wermuth, Wiley-VCH, Weinheim, Germany, 2002. Such salts can be prepared using the appropriate corresponding bases.

Pharmaceutically acceptable salts can be prepared by standard techniques. For example, the free-base form of a compound can be dissolved in a suitable solvent, such as an aqueous or aqueous-alcohol solution containing the appropriate acid and then isolated by evaporating the solution. Thus, if the particular compound is a base, the desired pharmaceutically acceptable salt may be prepared by any suitable method available in the art, for example, treatment of the free base with an inorganic acid, such as hydrochloric acid, hydrobromic acid, sulfuric acid, nitric acid, phosphoric acid and the like, or with an organic acid, such as acetic acid, maleic acid, succinic acid, mandelic acid, fumaric acid, malonic acid, pyruvic acid, oxalic acid, glycolic acid, salicylic acid, a pyranosidyl acid, such as glucuronic acid or galacturonic acid, an alpha-hydroxy acid, such as citric acid or tartaric acid, an amino acid, such as aspartic acid or glutamic acid, an aromatic acid, such as benzoic acid or cinnamic acid, a sulfonic acid, such as p-toluenesulfonic acid or ethanesulfonic acid, or the like.

Similarly, if the particular compound is an acid, the desired pharmaceutically acceptable salt may be prepared by any suitable method, for example, treatment of the free acid with an inorganic or organic base, such as an amine (primary, secondary or tertiary), an alkali metal hydroxide or alkaline earth metal hydroxide, or the like. Illustrative examples of suitable salts include organic salts derived from amino acids, such as L-glycine, L-lysine, and L-arginine, ammonia, primary, secondary, and tertiary amines, and cyclic amines, such as hydroxyethylpyrrolidine, piperidine, morpholine or piperazine, and inorganic salts derived from sodium, calcium, potassium, magnesium, manganese, iron, copper, zinc, aluminum and lithium.

It is also to be understood that the compounds of present disclosure can exist in unsolvated forms, solvated forms (e.g., hydrated forms), and solid forms (e.g., crystal or polymorphic forms), and the present disclosure is intended to encompass all such forms.

As used herein, the term “solvate” or “solvated form” refers to solvent addition forms that contain either stoichiometric or non-stoichiometric amounts of solvent. Some compounds have a tendency to trap a fixed molar ratio of solvent molecules in the crystalline solid state, thus forming a solvate. If the solvent is water the solvate formed is a hydrate; and if the solvent is alcohol, the solvate formed is an alcoholate. Hydrates are formed by the combination of one or more molecules of water with one molecule of the substance in which the water retains its molecular state as H₂O. Examples of solvents that form solvates include, but are not limited to, water, isopropanol, ethanol, methanol, DMSO, ethyl acetate, acetic acid, and ethanolamine.

As used herein, the terms “crystal form”, “crystalline form”, “polymorphic forms” and “polymorphs” can be used interchangeably, and mean crystal structures in which a compound (or a salt or solvate thereof) can crystallize in different crystal packing arrangements, all of which have the same elemental composition. Different crystal forms usually have different X-ray diffraction patterns, infrared spectral, melting points, density hardness, crystal shape, optical and electrical properties, stability and solubility. Recrystallization solvent, rate of crystallization, storage temperature, and other factors may cause one crystal form to dominate. Crystal polymorphs of the compounds can be prepared by crystallization under different conditions.

The present disclosure is also intended to include all isotopes of atoms in the compounds. Isotopes of an atom include atoms having the same atomic number but different mass numbers. For example, unless otherwise specified, hydrogen, carbon, nitrogen, oxygen, phosphorous, sulfur, fluorine, chlorine, bromide or iodine in the compounds of present disclosure are meant to also include their isotopes, such as but not limited to ¹H, ²H, ³H, ¹¹C, ¹²C, ¹³C, ¹⁴C, ¹⁴N, ¹⁵N, ¹⁶O, ¹⁷O, ¹⁸O, ³¹P, ³²P, ³²S, ³³S, ³⁴S, ³⁶S, ¹⁷F, ¹⁸F, ¹⁹F, ³⁵Cl, ³⁷Cl, ⁷⁹Br, ⁸¹Br, ¹²⁴I, ¹²⁷I and ¹³¹I. In some embodiments, hydrogen includes protium, deuterium and tritium. In some embodiments, carbon includes ¹²C and ¹³C.

### Synthesis of Compounds

Synthesis of the compounds provided herein, including pharmaceutically acceptable salts thereof, are illustrated in the synthetic schemes in the examples. The compounds provided herein can be prepared using any known organic synthesis techniques and can be synthesized according to any of numerous possible synthetic routes, and thus these schemes are illustrative only and are not meant to limit other possible methods that can be used to prepare the compounds provided herein. Additionally, the steps in the Schemes are for better illustration and can be changed as appropriate. The embodiments of the compounds in examples were synthesized for the purposes of research and potentially submission to regulatory agencies.

The reactions for preparing compounds of the present disclosure can be carried out in suitable solvents, which can be readily selected by one skilled in the art of organic synthesis. Suitable solvents can be substantially non-reactive with the starting materials (reactants), the intermediates, or products at the temperatures at which the reactions are carried out, e.g. temperatures that can range from the solvent's freezing temperature to the solvent's boiling temperature. A given reaction can be carried out in one solvent or a mixture of more than one solvent. Depending on the particular reaction step, suitable solvents for a particular reaction step can be selected by one skilled in the art.

Preparation of compounds of the present disclosure can involve the protection and deprotection of various chemical groups. The need for protection and deprotection, and the selection of appropriate protecting groups, can be readily determined by one skilled in the art. The chemistry of protecting groups can be found, for example, in T. W. Greene and P. G. M. Wuts, Protective Groups in Organic Synthesis, 3rd Ed., Wiley & Sons, Inc., New York (1999), which is incorporated herein by reference in its entirety.

Reactions can be monitored according to any suitable method known in the art. For example, product formation can be monitored by spectroscopic means, such as nuclear magnetic resonance spectroscopy (e.g. ¹H or ¹³C), infrared spectroscopy, spectrophotometry (e.g. UV-visible), mass spectrometry, or by chromatographic methods such as high performance liquid chromatography (HPLC), liquid chromatography-mass spectroscopy (LCMS), or thin layer chromatography (TLC). Compounds can be purified by one skilled in the art by a variety of methods, including high performance liquid chromatography (HPLC) (“Preparative LC-MS Purification: Improved Compound Specific Method Optimization” Karl F. Blom, Brian Glass, Richard Sparks, Andrew P. Combs J. Combi. Chem. 2004, 6(6), 874-883, which is incorporated herein by reference in its entirety), and normal phase silica chromatography.

The structures of the compounds in the examples are characterized by nuclear magnetic resonance (NMR) or/and liquid chromatography-mass spectrometry (LC-MS). NMR chemical shift (δ) is given in the unit of 10⁻⁶ (ppm). ¹H-NMR spectra is recorded in CDCl₃, CD₃OD or DMSO-d₆ solutions (reported in ppm) on a Varian instrument (400 MHz) or Brucker instrument (400 MHz), using tetramethylsilane (TMS) as the reference standard (0.0 ppm).

MS measurement is carried out using Shimadzu 2020 Mass Spectrometer using electrospray, chemical and electron impact ionization methods from a range of instruments.

TLC measurement is carried out using Shanghai Yu Cheng plates. The silica gel plates used for TLC are 0.15 mm˜0.2 mm. The silica gel plates used for separating and purifying products by TLC are 0.4 mm˜0.5 mm.

Column chromatography was done on a Biotage system (Manufacturer: Dyax Corporation) having a silica gel column or on a silica SepPak cartridge (Waters).

The known starting materials of the present disclosure can be synthesized by using or according to the known methods in the art, or can be purchased from commercial suppliers such as Adamas-beta, Bidepharm or Accela ChemBio Co., Ltd, and were used without further purification unless otherwise indicated. Tetrahydrofuran (THF), N,N-dimethylformamide (DMF), dichloromethane (DCM), dichloroethane (DCE), dioxane and 1,1,2,2-tetrachloroethane were purchased from Adamas-beta in Sure seal bottles and used as received.

Unless otherwise specified, the reactions of the present disclosure were all done under a positive pressure ofnitrogen or argon or with a drying tube in anhydrous solvents, and thereaction flasks were typically fitted with rubber septa for the introduction of substrates andreagents via syringe. Glassware was oven dried and/or heat dried.

For illustrative purposes, the following shows general synthetic route for preparing the compounds of the present disclosure as well as key intermediates. For a more detailed description of the individual reaction steps, see the Examples section below. Those skilled in the art will appreciate that other synthetic routes may be used to synthesize the inventive compounds. Although specific starting materials and reagents are depicted in the Schemes and discussed below, other starting materials and reagents can be easily substituted to provide a variety of derivatives and/or reaction conditions. In addition, many of the compounds prepared by the methods described below can be further modified in light of this disclosure using conventional chemistry well known to those skilled in the art.

### General Synthetic Route

In some embodiments, compounds of Formula (I) may be prepared by the reaction of a compound of Formula (II′)

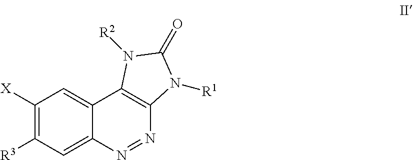

wherein X is a leaving group (for example a halogen atom such as a chlorine, an iodine, or a bromine atom, or a triflate group) with a compound of formula (III′):

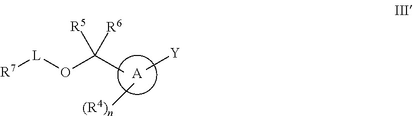

where Y is a boronic acid, boronic ester (e.g., boronic acid pinacol ester) or potassium trifluoroborate group. The reaction may be performed under standard conditions well known to those skilled in the art, for example in the presence of a palladium source (e.g., tetrakis triphenylphosphine palladium, palladium(II) acetate or [1,1′-bis(diphenylphosphino)ferrocene]dichloropalladium(II)), optionally a phosphine ligand (e.g., X-phos, Xantphos or S-phos), and a suitable base (e.g., cesium carbonate or triethylamine) at a suitable temperature.

In some embodiments, the compound of the Formula (II′) may be obtained by conventional procedures. Scheme 1 illustrates the synthesis of compounds of the Formula (II′).

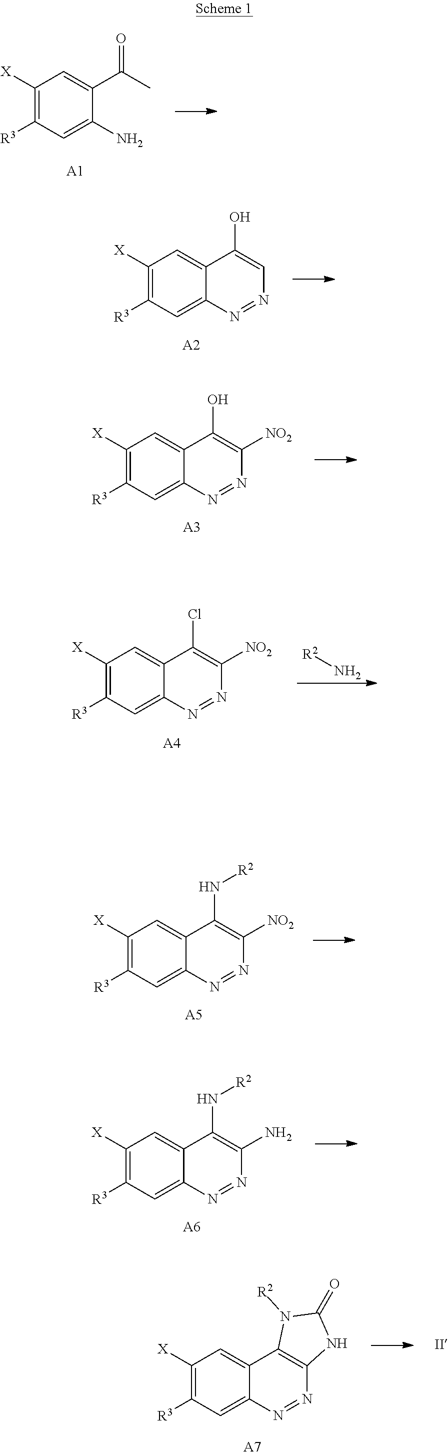

### Step 1

The starting material of Formula (A1) is commercially available or can be prepared using conventional methods, for example as described in WO2010/48582.

Compounds of Formula (A2) may be prepared by the cinnolin-4-ol cyclization reaction of a compound of Formula (A1) with diazotization reagents (e.g, NaNO₂ or isopentyl nitrite) under standard conditions.

### Step 2

Compounds of Formula (A3) may be prepared by the nitration reaction of a compound of Formula (A2) with nitration reagents (e.g., fuming nitric acid or concentrated nitric acid) under suitable conditions.

### Step 3

Compounds of Formula (A4) may be prepared by the halogenation reaction of a compound of Formula (A3) with halogenated reagents (e.g., POCl₃ or SOCl₂) in a suitable solvent (e.g., DMF) under standard conditions.

### Step 4

Compounds of Formula (A5) may be prepared by the nucleophilic substitution reaction of a compound of Formula (A4) with amine (e.g., primary amine or secondary amine) in a suitable solvent (e.g., THE or DMF) in the presence of an organic base (e.g., Et₃N or di-isopropylethylamine) under standard conditions.

### Step 5

Compounds of Formula (A6) may be prepared by the reduction reaction of a compound of Formula (A5) with reduction reagents (e.g., SnCl₂, or Fe/NH₄Cl, or H₂/palladium) under suitable reduction conditions.

### Step 6

Compounds of Formula (A7) may be prepared by the reaction of a compound of Formula (A6) with acylation reagent (e.g., 1,1′-Carbonyldiimidazole (CDI), ethyl carbonochloridate or bis(trichloromethyl) carbonate) under suitable coupling conditions.

### Step 7

Compounds of Formula (A7) can be reacted with suitable alkylation reagent (e.g., iodomethane or DMF-DMA), optionally in the presence of an appropriate base to provide compounds of Formula (II′).

In some embodiments, the compound of the Formula (III′) may be obtained by conventional procedures. Scheme 2 illustrates the synthesis of compounds of the Formula (III′).

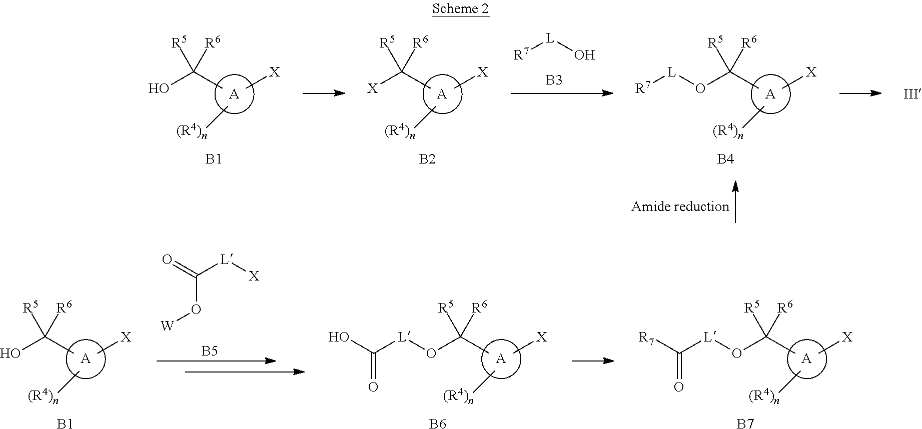

In Scheme 2, the compounds of Formula (III′) can be prepared starting from B1 which is either commercially available or synthesized from heteroaryl ethanone with Grignard reagents. Some of the compounds of Formula (III′) were prepared through the halogenation of B1 to obtain B2 with a halogenating agent such as thionyl chloride, phosphoryl chloride or a mixture of carbon tetrachloride and triphenylphosphine. After the alkylation of B3 by conventional procedures, the key intermediate B4 was finally reacted with common boron sources (for example, bis(pinacolato)diboron (B₂Pin₂), or bis(catecholato)diborane (B₂Cat₂)) under suitable transition metal catalyzed borylation reaction conditions to afford Formula (III′). Alternatively, intermediate B1 can be coupled with a suitable halide B5, where W is alkyl, followed by hydrolysis to afford free acid B6. Key intermediate B4 was also synthesized by amide reduction of B7, which was prepared by free acid B6 with R7 (amine) with proper coupling reagents.

### Use of Compound

In one aspect, the present disclosure provides compounds of Formula (I) or pharmaceutically acceptable salts thereof, which show ATM kinase inhibitory activity.

As used herein, the term “ATM kinase inhibitory activity” refers to a decrease in the activity of ATM kinase as a direct or indirect response to the presence of a compound of Formula (I), or pharmaceutically acceptable salt thereof, relative to the activity of ATM kinase in the absence of compound of Formula (I), or pharmaceutically acceptable salt thereof. Such a decrease in activity may be due to the direct interaction of the compound of Formula (I), or pharmaceutically acceptable salt thereof with ATM kinase, or due to the interaction of the compound of Formula (I), or pharmaceutically acceptable salt thereof with one or more other factors that in turn affect ATM kinase activity. For example, the compound of Formula (I), or pharmaceutically acceptable salt thereof may decrease ATM kinase by directly binding to the ATM kinase, by causing (directly or indirectly) another factor to decrease ATM kinase activity, or by (directly or indirectly) decreasing the amount of ATM kinase present in the cell or organism.

In some embodiments, the compounds of the present disclosure are selective inhibitors of ATM kinase.

As used herein, the term “selective inhibitor” or “selectively inhibits” means that a provided compound inhibits ATM kinase in at least one assy described herein (e.g., biochemical or cellular). In some embodiments, the term “selective inhibitor” or “selectively inhibits” means that a provided compound has the IC₅₀ for inhibiting the enzymes in PIKK family closely related to ATM kinase (such as PI3K, mTOR and ATR) at least 5000 fold higher, at least 4000 fold higher, at least 3000 fold higher, at least 2000 fold higher, at least 1000 fold higher, at least 500 fold higher, at least 400 fold higher, at least 300 fold higher, at least 200 fold higher, at least 100 fold higher, at least 90 fold higher, at least 80 fold higher, at least 70 fold higher, at least 60 fold higher, at least 50 fold higher, at least 40 fold higher, at least 30 fold higher, at least 20 fold higher, at least 10 fold higher, than the IC₅₀ for inhibiting ATM kinase.

In some embodiments, the compounds of the present disclosure are not AO substrates, as determined in human liver cytosol.

As used herein, the term “AO substrate” means that a given compound is susceptible to oxidation by aldehyde oxidase (“AO”) and thus highly susceptible to AO mediated clearance. In some embodiments, the AO susceptibility of a compound can be evaluated by intrinsic clearance (CLᵢₙₜ) in human liver cytosol system (Zientek M. et al, Drug Metab Dispos, 2010, 1322-27), as described in detail in Example section below. Human liver cytosol system useful in the evaluation is commercially available, for example, from Xenotech with catalog number H0606.C (AX) and lot number 1710130. In general, human liver cytosolic extracts can be prepared by ultra-centrifugation of liver homogenates obtained from human donors. In certain embodiments, the human liver cytosolic extracts (e.g. H0606.C (AX) from Xenotech) can be made specifically from donors with high AO activity to minimize underprediction of AO mediated clearance. PF-04217903 (2-[4-[3-(quinolin-6-ylmethyl)triazolo[4,5-b]pyrazin-5-yl]pyrazol-1-yl]ethanol, reported as a weak AO substrate) and Zaleplon (N-[3-(3-cyanopyrazolo[1,5-a]pyrimidin-7-yl)phenyl]-N-ethylacetamide, considered as a strong AO substrate) are both used as references in human liver cytosolic system. In general, a compound will not be considered as an AO substrate if the compound shows an CLᵢₙₜ lower than that of PF-04217903, while a compound will be considered as a strong AO substrate if the compound shows an CLᵢₙₜ higher than that of Zaleplon, wherein the CLᵢₙₜ is determined by the AO assay described in the AO assay described in Example section below.

In some embodiments, the compounds of the present disclosure show low AO susceptibility with a CLᵢₙₜ in human hepatocyte of less than that of PF-04217903. AOs are cytosolic molybdo-flavoenzymes, a group of proteins that require a flavin adenine dinucleotide (FAD) and a molybdopterin [molybdenum cofactor (MoCo)] for their catalytic activity. AOs oxidize aromatic aldehydes into the corresponding carboxylic acids and heterocycles into hydroxylated derivatives. The potential of AO to oxidize heterocycles is of paticular importance in the context of drug design and development, as these chemical groups are popular synthetic blocks in medicinal chemistry. AO mediated metabolism is often overlooked during drug discovery stage, and high clearance issue is not revealed until in phase 1 clinical trial. AO is a cytosolic enzyme and thus its potential contribution to the metabolic clearance of new compounds is not addressed in standard metabolic stability screens using liver microsomes. Hepatocytes are a whole cell system which contains both microsomes and cytosol. However, AO is an unstable protein with substantial loss in activity during hepatocytes preparation (Hutzler, J. M. et al., Drug Metab Dispos, 2014, 1090-7). In vivo studies on AO mediated metabolism in animal models are also highly problematic, as the components liver AOs in humans and in popular experimental animals are different (Garattini, E. et al., Expert Opin Drug Discovery, 2013, 641-54). Human liver is characterized by a single and active AO isozyme, that is, AOX1. The predominant AOX form expressed in many mouse and rat strains is AOX3. Two other experimental animals, cats and dogs, are characterized by absence of AO enzymatic activity. AO activity has been found to be much more active in higher primates (humans and monkeys) compared to rodents. AO is very concentrated in the liver, where it oxidizes multiple aldehydes and nitrogenous heterocyclic compounds, such as anti-cancer andimmunosuppressive drugs (see, for example, Gordon A H, Green D E, Subrahmanyan, “Liver aldehyde oxidase”, The Biochemical Journal. 1940, 34(5): 764-74). Human liver cytosolic extracts, which contain AO, but not significant amount of contaminating CYP450, has been shown to be a valuable tool to predict in vivo clearance mediated by human AO. Human liver cytosol purchased from Xenotech was used immediately after thawing, and not reused as AO is an unstable enzyme and becomes rapidly inactivated upon freezing-thawing. In light of highly variable AO activity, a high activity lot of human liver cytosol, was chosen for AO assay to minimize underprediction of clearance. Reference compounds Zaleplon (high clearance by AO) and PF-04217903 (low clearance by AO) were used as controls in the AO assay.

Without wishing to be bound by any particular theory, it is believed that AO has a significant impact on pharmacokinetics. AO is capable of oxidizing many drugs in the liver, because of its broad substrate specificity (Strelevitz T J, Orozco C C, Obach R S. “Hydralazine as a selective probe inactivator of aldehyde oxidase in human hepatocytes: estimation of the contribution of aldehyde oxidase to metabolic clearance”. Drug Metabolism and Disposition. 2012, 40 (7): 1441-8). AO greatly contributes to the hepatic clearance of drugs and other compounds (Hartmann T, Terao M, Garattini E, Teutloff C, Alfaro J F, Jones J P, Leimkuhler S. “The impact of single nucleotide polymorphisms on human aldehyde oxidase”, Drug Metabolism and Disposition. 2012, 40 (5): 856-64). AO mediated metabolism tends to lead to high clearance in humans. For high clearance compounds, small change in intrinsic clearance due to different enzyme expression level among patients causes large change of bioavailability. The human AOX1 is highly polymorphic and some inactivating missense as well as nonsense polymorphic sites have been described in the human population (Garattini, E. et al, Expert Opin Drug Discovery, 2012, 487-503; Hartmann, T. et al, Drug Metab Dispos, 2012, 856-64). Such polymorphism results in reduced levels of the encoded AOX1 protein and explains the reported interindividual variability in AOX activity. Additionally many factors may affect AO activity, such as gender, age, cigarette smoking, drug usage, and disease states. Therefore, compounds with AO mediated high clearance have large inter-patients PK variability which results in unexpected toxicities in some individuals whereas efficacy is not achieved in other patients (Garattini, E. et al, Expert Opin Drug Discovery, 2013, 641-54; Hutzler, J. M. et al, Drug Metab Dispos 2014, 1090-7).

In contrast to the previously reported ATM inhibitors AZD0156 and AZD1390 that are strong AO substrate, the compounds of the present disclosure have surprisingly low susceptibility to AO oxidation. Therefore, in one aspect, the compounds and pharmaceutically acceptable salts thereof provided herein are not AO substrates, and consequently show better PK profile than compounds that are AO substrates. For example, the compounds provided herein have low PK variability, among humans that have different levels of AO activity.

In some embodiments, the compounds of the present disclosure show good solubility in water. In some embodiments, the compounds of the present disclosure show a solubility in water of above 90 μM, above 100 μM, above 200 μM, above 300 μM, above 400 μM, above 500 μM, above 600 μM, above 700 μM, above 800 μM, above 900 μM, or above 1000 μM.

As a result of their ATM kinase inhibitory activity (optionally selective ATM kinase inhibitory activity), the compounds of Formula (I), and pharmaceutically acceptable salts thereof are useful in therapy, for example in the treatment of diseases or medical conditions mediated at least in part by ATM kinase, including cancer.

As used herein, the term “cancer” is intented to encompass both non-metastatic cancer and metastatic cancer. In this context, treating cancer involves treatment of both primary tumors and tumor metastases.

As used herein, the term “therapy” is intended to have its normal meaning of dealing with a disease in order to entirely or partially relieve one, some or all of its symptoms, or to correct or compensate for the underlying pathology. The term “therapy” also includes “prophylaxis” unless there are specific indications to the contrary. The terms “therapeutic” and “therapeutically” should be interpreted in a corresponding manner.

As used herein, the term “prophylaxis” is intended to have its normal meaning and includes primary prophylaxis to prevent the development of the disease and secondary prophylaxis whereby the disease has already developed and the patient is temporarily or permanently protected against exacerbation or worsening of the disease or the development of new symptoms associated with the disease.

The term “treatment” is used synonymously with “therapy”. Similarly the term “treat” can be regarded as “applying therapy” where “therapy” is as defined herein.

Therefore, in one aspect, there is provided a compound of Formula (I), or a pharmaceutically acceptable salt thereof, for use in therapy.

In some embodiments, there is provided a compound of Formula (I), or a pharmaceutically acceptable salt thereof, for use as a medicament.

In some embodiments, there is provided a compound of Formula (I), or a pharmaceutically acceptable salt thereof, for use in the treatment of ATM-associated diseases or conditions. In some embodiments, the ATM-associated disease or condition is cancer. In some embodiments, the cancer is selected from the group consisting of colorectal cancer, glioblastoma, gastric cancer, ovarian cancer, diffuse large B-cell lymphoma, chronic lymphocytic leukaemia, acute myeloid leukaemia, head and neck squamous cell carcinoma (including but not limited to lip carcinoma, oral cavity carcinoma, oropharynx carcinoma, hypopharynx carcinoma, glottic larynx carcinoma, supraglottic larynx carcinoma, ethmoid sinus carcinoma, maxillary sinus carcinoma, and occult primary carcinoma), breast cancer, hepatocellular carcinoma, small cell lung cancer and non-small cell lung cancer.

In some embodiments, there is provided a compound of Formula (I), or a pharmaceutically acceptable salt thereof, for use in the manufacture of a medicament for the treatment of ATM-associated diseases or conditions.

In some embodiments, there is provided a compound of Formula (I), or a pharmaceutically acceptable salt thereof, for use in the manufacture of a medicament for the treatment of cancer.

### Pharmaceutical Composition

The present disclosure provides pharmaceutical compositions comprising one or more compound of the present disclosure, or a pharmaceutically acceptable salt thereof. In some embodiments, the pharmaceutical composition comprises one or more compounds of the present disclosure, or a pharmaceutically acceptable salt thereof, and at lease one pharmaceutical acceptable excipient.

A “pharmaceutical composition”, as used herein, is a formulation containing the compounds of the present disclosure in a form suitable for administration to a subject. In some embodiments, the pharmaceutical composition is in bulk or in unit dosage form. The unit dosage form is any of a variety of forms, including, for example, tablets, capsules, pills, powders, granules, sachets, cachets, lozenges, suspensions, emulsions, solutions, syrups, aerosols (as a solid or in a liquid medium), spray, ointment, paste, cream, lotion, gel, patch, inhalant, or suppository. The quantity of active ingredient (e.g., a formulation of the disclosed compound or salt, hydrate, solvate or isomer thereof) in a unit dose of composition is a therapeutically effective amount and is varied according to the particular treatment involved. One skilled in the art will appreciate that it is sometimes necessary to make routine variations to the dosage depending on the age and condition of the patient. The dosage will also depend on the route of administration. A variety of routes are contemplated, including oral, pulmonary, rectal, parenteral, transdermal, subcutaneous, intravenous, intramuscular, intraperitoneal, inhalational, buccal, sublingual, intrapleural, intrathecal, intranasal, and the like. Dosage forms for the topical or transdermal administration of a compound of this invention include powders, sprays, ointments, pastes, creams, lotions, gels, solutions, patches and inhalants. In some embodiments, the compound of the present disclosure is mixed under sterile conditions with a pharmaceutically acceptable excipient, and with any preservatives, buffers or propellants that are required.

As used herein, the term “pharmaceutically acceptable excipient” means an excipient that is useful in preparing a pharmaceutical composition that is generally safe, non-toxic and neither biologically nor otherwise undesirable, and includes excipient that is acceptable for veterinary use as well as human pharmaceutical use. A “pharmaceutically acceptable excipient” as used in the specification and claims includes both one and more than one such excipient.

As used herein, the term “therapeutically effective amount” refers to an amount of a pharmaceutical agent to treat, ameliorate, or prevent an identified disease or condition, or to exhibit a detectable therapeutic or inhibitory effect. The effect can be detected by any assay method known in the art. The precise effective amount for a subject will depend upon the subject's body weight, size, and health; the nature and extent of the condition; and the therapeutic or combination of therapeutics selected for administration. Therapeutically effective amounts for a given situation can be determined by routine experimentation that is within the skill and judgment of the clinician.

In some embodiments, the pharmaceutical compositions can be formulated so that a dosage of between 0.01-500 mg/kg body weight/day, for example, 0.05-500 mg/kg body weight/day, 0.1-500 mg/kg body weight/day, 0.1-400 mg/kg body weight/day, 0.1-300 mg/kg body weight/day, 0.1-200 mg/kg body weight/day, 0.1-100 mg/kg body weight/day, 0.1-80 mg/kg body weight/day, 1-100 mg/kg body weight/day or 1-80 mg/kg body weight/day of the compounds of the present disclosure, or a pharmaceutically acceptable salt thereof, can be administered.

In some embodiments, the pharmaceutical compositions comprise one or more compounds of the present disclosure, or a pharmaceutically acceptable salt thereof, as a first active ingredient, and further comprise a second active ingredient. The second active ingredient can be any anti-tumor agent known in the art, for example, antineoplastic agents, antiangiogenic agents, immunotherapy approaches, efficacy enhancers, and the like.

Examples of the antineoplastic agents include, but are not limited to, DNA alkylating agents (for example cisplatin, oxaliplatin, carboplatin, cyclophosphamide, nitrogen mustards like ifosfamide, bendamustine, melphalan, chlorambucil, busulphan, temozolamide and nitrosoureas like carmustine); antimetabolites (for example gemcitabine and antifolates such as fluoropyrimidines like 5-fluorouracil and tegafur, raltitrexed, methotrexate, cytosine arabinoside, and hydroxyurea); anti-tumor antibiotics (for example anthracyclines like adriamycin, bleomycin, doxorubicin, liposomal doxorubicin, pirarubicin, daunomycin, valrubicin, epirubicin, idarubicin, mitomycin, dactinomycin, amrubicin and mithramycin); antimitotic agents (for example vinca alkaloids like vincristine, vinblastine, vindesine and vinorelbine and taxoids like taxol and taxotere and polokinase inhibitors); and topoisomerase inhibitors (for example epipodophyllotoxins like etoposide and teniposide, amsacrine, irinotecan, topotecan and camptothecin); inhibitors of DNA repair mechanisms such as CHK kinase; DNA-dependent protein kinase inhibitors; inhibitors of poly (ADP-ribose) polymerase (PARP inhibitors, including Olaparib, Rucaparib, Niraparib, Talazoparib, Pamiparib and Fluzoparib); and Hsp90 inhibitors such as tanespimycin and retaspimycin, inhibitors of ATR kinase (such as AZD6738); and inhibitors of WEE 1 kinase (such as AZD1775/MK-1775).

Examples of antiangiogenic agents include those that inhibit the effects of vascular endothelial growth factor, such as but not limited to, the anti-vascular endothelial cell growth factor antibody bevacizumab, a VEGF receptor tyrosine kinase inhibitor such as vandetanib (ZD6474), sorafenib, vatalanib (PTK787), sunitinib (SU11248), axitinib (AG-013736), pazopanib (GW 786034) and cediranib (AZD2171); compounds such as those disclosed in International Patent Applications WO 97/22596, WO 97/30035, WO 97/32856 and WO 98/13354; and compounds that work by other mechanisms (for example linomide, inhibitors of integrin αvβ3 function and angiostatin), or inhibitors of angiopoietins and their receptors (Tie-1 and Tie-2), inhibitors of PLGF, inhibitors of delta-like ligand (DLL-4).

Examples of immunotherapy approaches include, but are not limited to, ex-vivo and in-vivo approaches to increase the immunogenicity of patient tumor cells, such as transfection with cytokines such as interleukin 2, interleukin 4 or granulocyte-macrophage colony stimulating factor; approaches to decrease T-cell anergy or regulatory T-cell function; approaches that enhance T-cell responses to tumors, such as blocking antibodies to CTLA4 (for example ipilimumab and tremelimumab), B7H1, PD-1 (for example BMS-936558 or AMP-514), PD-L1 (for example MEDI4736) and agonist antibodies to CD 137; approaches using transfected immune cells such as cytokine-transfected dendritic cells; approaches using cytokine-transfected tumor cell lines, approaches using antibodies to tumor associated antigens, and antibodies that deplete target cell types (e.g., unconjugated anti-CD20 antibodies such as Rituximab, radiolabeled anti-CD20 antibodies Bexxar and Zevalin, and anti-CD54 antibody Campath); approaches using anti-idiotypic antibodies; approaches that enhance Natural Killer cell function; and approaches that utilize antibody-toxin conjugates (e.g. anti-CD33 antibody Mylotarg); immunotoxins such as moxetumomab pasudotox; agonists of toll-like receptor 7 or toll-like receptor 9.

Examples of efficacy enhancers include leucovorin.

Therefore, in some embodiments, there is provided pharmaceutical composition comprising a compound of Formula (I), or a pharmaceutically acceptable salt thereof, and at least one additional anti-tumor agent. In some embodiments, there is one additional anti-tumor agent. In some embodiments, there are two additional anti-tumor agents. In some embodiments, there are three or more additional anti-tumor agents.

In some embodiments, the amount of additional anti-tumor agent present in the composition of the present disclosure can be no more than the amount that would normally be administered in a composition comprising that anti-tumor agent as the only active agent. In certain embodiments, the amount of the additional anti-tumor agent in the composition of the present disclosure will range from about 50% to 100% of the amount normally present in a composition comprising that anti-tumor agent as the only therapeutically active agent.

Therefore, in another aspect, there is provided a compound of formula (I) or a pharmaceutically acceptable salt thereof in combination with one or more anti-tumor agents listed above.

In some embodiments, the additional anti-tumor agent is selected from the group consisting of doxorubicin, irinotecan, topotecan, etoposide, mitomycin, bendamustine, chlorambucil, cyclophosphamide, ifosfamide, carmustine, melphalan and bleomycin.

As used herein, the term “combination” refers to simultaneous, separate or sequential administration. In some embodiments, “combination” refers to simultaneous administration. In some embodiments, “combination” refers to separate administration. In some embodiments, “combination” refers to sequential administration. Where the administration is sequential or separate, the delay in administering the second component should not be such as to lose the beneficial effect of the combination.

In a further aspect, there is provided a pharmaceutical composition comprising a compound of formula (I) or a pharmaceutically acceptable salt thereof in combination with one or more anti-tumor agents listed above, in association with a pharmaceutically acceptable excipient.

In a further aspect, there is provided a kit comprising a compound of formula (I) or a pharmaceutically acceptable salt thereof in combination with one or more anti-tumor agents listed above.

In a further aspect, there is provided a kit comprising:
(a) a compound of formula (I) or a pharmaceutically acceptable salt thereof in a first unit dosage form;
(b) an anti-tumor agent selected from those listed above in a second unit dosage form; and
(c) container for containing the first and second unit dosage forms.

### Method for Treatment

In a further aspect, there is provided a method of treating ATM-associated diseases or conditions in a subject in need thereof, which comprises administering to the subject a therapeutically effective amount of a compound of Formula (I) or a pharmaceutically acceptable salt thereof, or a pharmaceutical composition of the present disclosure, owning to the selective ATM kinase inhibitory activity and non-AO liability of the compounds of the present disclosure.

In some embodiments, the ATM-associated disease or condition is cancer. In some embodiments, the cancer is selected from the group consisting of colorectal cancer, glioblastoma, gastric cancer, ovarian cancer, diffuse large B-cell lymphoma, chronic lymphocytic leukaemia, acute myeloid leukaemia, head and neck squamous cell carcinoma, breast cancer, hepatocellular carcinoma, small cell lung cancer and non-small cell lung cancer. In some embodiments, the cancer is head and neck squamous cell carcinoma, including but not limited to, lip carcinoma, oral cavity carcinoma, oropharynx carcinoma, hypopharynx carcinoma, glottic larynx carcinoma, supraglottic larynx carcinoma, ethmoid sinus carcinoma, maxillary sinus carcinoma, and occult primary carcinoma. In some embodiments, the cancer is metastatic cancer. In some embodiments, the metastatic cancer comprises metastases of the central nervous system. In some embodiments, the metastases of the central nervous system comprise brain metastases. In some embodiments, the metastases of the central nervous system comprise leptomeningeal metastases. “Leptomeningeal metastases” occur when cancer spreads to the meninges, the layers of tissue that cover the brain and the spinal cord. Metastases can spread to the meninges through the blood or they can travel from brain metastases, carried by the cerebrospinal fluid (CSF) that flows through the meninges.

As used herein, the term “subject in need thereof” is a subject having an ATM-associated disease or condition (e.g., cancer), or a subject having an increased risk of developing an ATM-associated disease or condition (e.g., cancer) relative to the population at large. In the case of cancer, a subject in need thereof can have a precancerous condition. A “subject” includes a warm-blooded animal. In some embodiments, the warm-blooded animal is a human.

In this context, the term “therapeutically effective amount” refers to an amount of a compound of Formula (I) or pharmaceutically acceptable salts thereof which is effective to provide “therapy” in a subject, or to “treat” an ATM-associated disease or disorder in a subject. In the case of cancer, the therapeutically effective amount may cause any of the changes observable or measurable in a subject as described in the definition of “therapy”, “treatment” and “prophylaxis” above. For example, the effective amount can reduce the number of cancer or tumor cells; reduce the overall tumor size; inhibit or stop tumor cell infiltration into peripheral organs including, for example, the soft tissue and bone; inhibit and stop tumor metastasis; inhibit and stop tumor growth; relieve to some extent one or more of the symptoms associated with the cancer; reduce morbidity and mortality; improve quality of life; or a combination of such effects. An effective amount may be an amount sufficient to decrease the symptoms of a disease responsive to inhibition of ATM kinase activity. For cancer therapy, efficacy in-vivo can, for example, be measured by assessing the duration of survival, time to disease progression (TTP), the response rates (RR), duration of response, and/or quality of life. As recognized by those skilled in the art, effective amounts may vary depending on route of administration, excipient usage, and co-usage with other agents. For example, where a combination therapy is used, the amount of the compound of formula (I) or pharmaceutcially acceptable salt described in this specification and the amount of the other pharmaceutically active agent(s) are, when combined, jointly effective to treat a targeted disorder in the animal patient. In this context, the combined amounts are in a “therapeutically effective amount” if they are, when combined, sufficient to decrease the symptoms of a disease responsive to inhibition of ATM activity as described above.

In generally, “therapeutically effective amount” may be determined by one skilled in the art by, for example, starting with the dosage range described in this specification for the compound of formula (I) or pharmaceutcially acceptable salt thereof and an approved or otherwise published dosage range(s) of the other pharmaceutically active compound(s).

The method of treating ATM-associated diseases or conditions described in this specification may be used as a monotherapy. As used herein, the term “monotherapy” refers to the administration of a single active or therapeutic compound to a subject in need thereof. In some embodiments, monotherapy will involve administration of a therapeutically effective amount of one of the compounds of the present disclosure, or a pharmaceutically acceptable salt thereof, to a subject in need of such treatment.

Depending upon the particular diseases or conditions to be treated, the method of treating ATM-associated diseases or conditions described in this specification may involve, in addition to administration of the compound of Formula (I), one or more additional therapies, for example, conventional surgery, radiotherapy, chemotherapy, or a combination of such additional therapies. As used herein, the term“combination therapy” refers to the administration of a combination of multiple active compounds.

The additional therapies, such as additional anti-tumor agents, may be administered separately from the compounds of the present disclosure, as part of a multiple dosage regimen. Alternatively, these additional therapies may be part of a single dosage form, mixed with the compounds of the present disclosure in a single composition.

In some embodiments, the compounds of the present disclosure may be administered simultaneously, sequentially or separately to treatment with the conventional surgery, radiotherapy or chemotherapy.

Radiotherapy may include one or more of the following categories of therapy: (i) external radiation therapy using electromagnetic radiation, and intraoperative radiation therapy using electromagnetic radiation; (ii) internal radiation therapy or brachytherapy; including interstitial radiation therapy or intraluminal radiation therapy; or (iii) systemic radiation therapy, including but not limited to iodine 131 and strontium 89.

Chemotherapy may include anti-tumor agents known in the art, for example, antineoplastic agents, antiangiogenic agents, immunotherapy approaches, efficacy enhancers, and the like described in this specification.

Therefore, in one aspect, there is provided a method of treating ATM-associated diseases or conditions in a subject in need thereof, wherein the compound of Formula (I) or pharmaceutically acceptable salts thereof is administered simultaneously, separately or sequentially with radiotherapy.

In some embodiments, the radiotherapy is brain radiation.

In some embodiments, the ATM-associated disease or condition is cancer. In some embodiments, the cancer is selected from glioblastoma, lung cancer (for example small cell lung cancer or non-small cell lung cancer), breast cancer (for example triple negative breast cancer), head and neck squamous cell carcinoma (for example lip carcinoma, oral cavity carcinoma, oropharynx carcinoma, hypopharynx carcinoma, glottic larynx carcinoma, supraglottic larynx carcinoma, ethmoid sinus carcinoma, maxillary sinus carcinoma, or occult primary carcinoma), oesophageal cancer, cervical cancer and endometrial cancer. In some embodiments, the cancer is glioblastoma. In some embodiment, the cancer is metastatic cancer. In some embodiments, the metastatic cancer is metastases of the central nervous system. In some embodiments, the metastases of the central nervous system is brain metastases.

In some embodiments, there is provided a method of treating glioblastoma in a subject in need thereof, wherein the compound of Formula (I) or pharmaceutically acceptable salts thereof is administered simultaneously, separately or sequentially with brain radiation.

In another aspect, there is provided a method of treating ATM-associated diseases or conditions in a subject in need thereof, wherein the compound of Formula (I) or pharmaceutically acceptable salts thereof is administered simultaneously, separately or sequentially with one or more additional anti-tumor agents.

In some embodiments, the ATM-associated disease or condition is cancer. In certain embodiments, the amounts of the compound of Formula (I), or a pharmaceutically acceptable salt thereof, and the one or more additional anti-tumor agents are jointly effective in producing an anti-cancer effect.

In some embodiments, the additional anti-tumor agent includes antineoplastic agents, antiangiogenic agents, immunotherapy approaches, efficacy enhancers and the like.

In some embodiments, the additional anti-tumor agent is selected from the group consisting of doxorubicin, irinotecan, topotecan, etoposide, mitomycin, bendamustine, chlorambucil, cyclophosphamide, ifosfamide, carmustine, melphalan and bleomycin.

In some embodiments, the compounds of the present disclosure may be administered simultaneously, sequentially or separately with antineoplastic agents.

In certain embodiments, the antineoplastic agents are PARP inhibitors. In certain embodiments, the PARP inhibitors are selected from the group consisting of Olaparib, Rucaparib, Niraparib, Talazoparib, Pamiparib and Fluzoparib.

### EXAMPLES

For the purpose of illustration, the following examples are included. However, it is to be understood that these examples do not limit the invention and are only meant to suggest a method of practicing the present disclosure. Persons skilled in the art will recognize that the chemical reactions described may be readily adapted to prepare a number of other compounds of the present disclosure, and alternative methods for preparing the compounds of the present disclosure are deemed to be within the scope of the present disclosure. For example, the synthesis of non-exemplified compounds according to the present disclosure may be successfully performed by modifications apparent to those skilled in the art, e.g., by appropriately protecting interfering groups, by utilizing other suitable reagents known in the art other than those described, and/or by making routine modifications of reaction conditions. Alternatively, other reactions disclosed herein or known in the art will be recognized as having applicability for preparing other compounds of the present disclosure.

The following abbreviations have been used in the examples:

| | |
|---|---|
| AlCl₃ | aluminum chloride |
| aq. | aqueous |
| BH₃ | Borane |
| CDI | N,N′-carbonyldiimidazole |
| CH₂Cl₂ | dichloromethane |
| conc. | concentrated |
| DCE | 1,2-dichloroethane |
| DCM | dichloromethane |
| DIEA or | diisopropylethylamine |
| DIPEA | |
| DMF | N,N-dimethylformamide |
| DMSO | dimethyl sulfoxide |
| T₃P | propanephosphonic acid anhydride |
| Et₃N or TEA | triethylamine |
| EtOAc | ethyl acetate |
| HNO₃ | nitric acid |
| H₂SO₄ | sulfuric acid |
| HCl | hydrochloric acid |
| hr(s) | hour(s) |
| K₂CO₃ | potassium carbonate |
| KOAc | potassium acetate |
| MeCN | acetonitrile |
| MeMgBr | methylmagnesium bromide |
| MeI | methyl iodide |
| MeOH | methanol |
| MTBE | methyl tert-butyl ether |
| NaH | sodium hydride |
| NaHCO₃ | sodium bicarbonate |
| NaNO₂ | sodium nitrite |
| NaOH | sodium hydroxide |
| Na₂SO₄ | sodium sulfate |
| NH₄Cl | ammonium chloride |
| Pd(dppf)Cl₂ | [1,1′-bis(diphenylphosphino)ferrocene]dichloropalladium(II) |
| PE | petroleium ether |
| POCl₃ | phosphoric trichloride |
| Pd/C | palladium on carbon |
| SOCl₂ | thionyl chloride |
| t-BuONa | sodium tert-butoxide |
| TFA | trifluoroacetic acid |
| THF | tetrahydrofuran |
| TMSCF₃ | trifluoromethyltrimethylsilane |
| TMSOTF | trimethylsilyl trifluoromethanesulfonate |

### Example 1

### 8-(6-((2-(3-azabicyclo[3.1.0]hexan-3-yl)ethoxy)methyl)pyridin-3-yl)-1-isopropyl-3-methyl-1H-imidazo[4,5-c]cinnolin-2(3H)-one

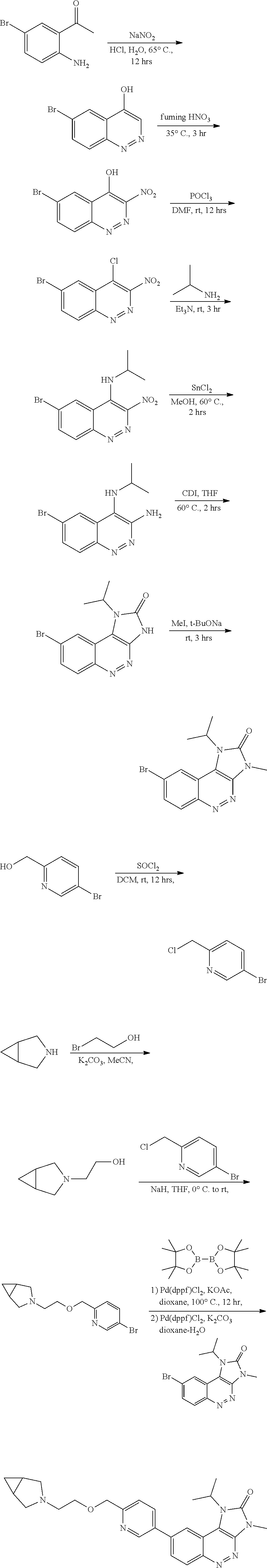

### Step 1: 6-bromocinnolin-4-ol

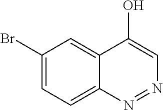 [SMILES: Oc1cnnc2ccc(Br)cc12]

1-(2-amino-5-bromophenyl)ethenone (200 g, 935 mmol) was slowly dissolved in con. HCl (2 L) and water (440 mL) below 10° C., and the mixture was stirred at 0° C. for 1 h. Then to the mixture was added dropwise a solution of NaNO₂ (71.0 g, 1.03 mol) in water (360 mL) below 0° C. and the resulting reaction mixture was stirred at 0° C. for another 1 h. Then the reaction mixture was stirred at 65° C. for 16 h. After cooled to room temperature, the mixture was poured into ice water (1 L) and filtered. The filtered residue was dried in oven at 55° C. for 24 h to give desired product (174 g, 82.7% yield) as a yellow solid. ¹H NMR (400 MHz, DMSO-d₆) δ 14.09 (s, 1H), 8.12 (s, 1H), 7.95 (d, J=8.0 Hz, 1H), 7.83 (s, 1H), 7.73 (d, J=8.4 Hz, 1H). LC-MS (ESI) m z: 225 [M+H]⁺.

### Step 2: 6-bromo-3-nitrocinnolin-4-ol

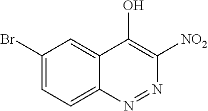 [SMILES: O=[N+]([O-])c1nnc2ccc(Br)cc2c1O]

6-bromocinnolin-4-ol (50.0 g) was slowly dissolved in fuming HNO₃ (200 mL) at 20° C.-35° C. and the reaction mixture was stirred at 28° C.-33° C. for 5 h. The mixture was poured into ice water (500 mL) and filtered. The filtered residue in EtOH (500 mL) was heated at 85° C. for about 2 h until the solution was clear. After cooled to 10° C., the mixture was filtered and the filter cake was dried in oven at 50° C. for 16 h to give the desired product (35.0 g, 58.3% yield) as a yellow solid. LC-MS (ESI) m z: 270 [M+H]⁺.

### Step 3: 6-bromo-4-chloro-3-nitrocinnoline

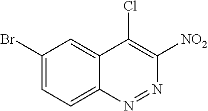 [SMILES: O=[N+]([O-])c1nnc2ccc(Br)cc2c1Cl]

To a solution of 6-bromo-3-nitrocinnolin-4-ol (100 g, 372 mmol) in dry DMF (1 L) was added dropwise POCl₃ (51.0 mL, 558 mmol) at 0° C. under N₂ and the reaction mixture was stirred at 10° C.-15° C. for 12 h. The mixture was poured into ice water (1.5 L) and filtered. The filtered residue was triturated with MTBE (500 mL) for 1 h at room temperature and filtered. The filtered cake was dried in oven at 40° C. for 16 h to give the desired product (90.0 g, 84.3% yield) as a yellow solid. LC-MS (ESI) m z: 288 [M+H]⁺.

### Step 4: 6-bromo-N-isopropyl-3-nitrocinnolin-4-amine

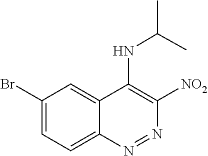 [SMILES: CC(C)Nc1c([N+](=O)[O-])nnc2ccc(Br)cc12]

To a mixture of 6-bromo-4-chloro-3-nitrocinnoline (50.0 g, 174 mmol) and propan-2-amine (15.4 g, 261 mmol) in DCM (500 mL) was added TEA (77.4 mL, 522 mmol) and the reaction mixture was stirred at room temperature for 3 h. The mixture was poured into ice 1 M HCl solution (1 L) and extracted with DCM (500 mL×2). The combined organic layers were washed with brine (800 mL), dried over anhydrous Na₂SO₄, filtered and the filtrate was concentrated under reduced pressure to give the desired product (50.0 g, 79.8% yield) as a brown solid. LC-MS (ESI) m z: 311 [M+H]⁺.

### Step 5: 6-bromo-N4-isopropylcinnoline-3,4-diamine

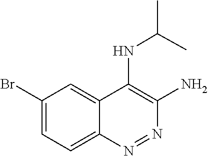 [SMILES: CC(C)Nc1c(N)nnc2ccc(Br)cc12]

To a solution of 6-bromo-N-isopropyl-3-nitrocinnolin-4-amine (75.0 g, 242 mmol) in MeOH (400 mL) was added SnCl₂·H₂O (164 g, 726 mmol) at 0° C. and the reaction mixture was stirred at 60° C. for 2 h. The mixture was adjusted with 50% NaOH solution until pH˜8 below 45° C. and stirred at 55° C. for 45 min. The mixture was filtered and the filtered residue was triturated with THE (300 mL) for 1 h at room temperature. It was filtered and the combined filtrate was evaporated under reduced pressure. The residue was triturated with THF (300 mL) for 1 h at room temperature and filtered again. The filtrate was evaporated under reduced pressure to give the desired product (50.0 g, 73.8% yield) as a dark brown solid. LC-MS (ESI) m z: 281 [M+H]⁺.

### Step 6: 8-bromo-1-isopropyl-1H-imidazo[4,5-c]cinnolin-2(3H)-one (8)

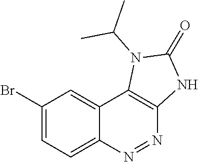 [SMILES: CC(C)n1c(=O)[nH]c2nnc3ccc(Br)cc3c21]

To a solution of 6-bromo-N4-isopropylcinnoline-3,4-diamine (49.0 g, 175 mmol) in dry THE (500 mL) was added CDI (85.0 g, 525 mmol) and the reaction mixture was stirred at room temperature for 2 h. The mixture was evaporated under reduced pressure and the residue was dissolved in water (250 mL). It was adjusted with 1 M HCl solution until pH˜7 and filtered. The filtered residue was dried in oven at 55° C. for 16 h to give the desired product (40.0 g, 74.6% yield). LC-MS (ESI) m z: 307 [M+H]⁺.

### Step 7: 8-bromo-1-isopropyl-3-methyl-1H-imidazo[4,5-c]cinnolin-2(3H)-one

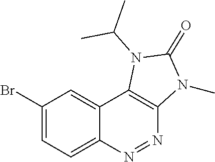 [SMILES: CC(C)n1c(=O)n(C)c2nnc3ccc(Br)cc3c21]

To a solution of 8-bromo-1-isopropyl-1H-imidazo[4,5-c]cinnolin-2(3H)-one (40.0 g, 131 mmol) in dry DMF (300 mL) was added t-BuONa (15.0 g, 157 mmol) at 0° C. and stirred at 0° C. for 30 min under N₂. To the reaction mixture was added dropwise Mel (37.2 g, 262 mmol) at 10° C.-15° C. and the reaction mixture was stirred at 10° C.-15° C. for 2 h. The mixture was poured into ice water (600 mL) and filtered. The filtered residue was triturated with MeOH (200 mL) for 1 h and filtered. The filtered cake was dried under vacuum to give the desired product (20.0 g, 47.8% yield). ¹H NMR (400 MHz, CDCl₃) δ 8.34 (d, J=9.2 Hz, 1H), 8.28 (d, J=1.4 Hz, 1H), 7.73 (dd, J=9.2, 1.8 Hz, 1H), 5.21-5.02 (m, 1H), 3.75 (s, 3H), 1.77 (d, J=6.9 Hz, 6H). LC-MS (ESI) m z: 321 [M+H]⁺.

### Step 8: 5-bromo-2-(chloromethyl)pyridine

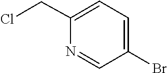

To a solution of (5-bromopyridin-2-yl) methanol (5.00 g, 26.7 mmol) in DCM (50 mL) was added SOCl₂ (3.87 mL, 53.4 mmol) slowly and the resulting reaction mixture was stirred at room temperature for 2 h. The mixture was evaporated under reduced pressure. The residue was dissolved in DCM (50 mL), washed with sat. NaHCO₃ solution (50 mL). The organic layer was dried over anhydrous Na₂SO₄, filtered and the filtrate was concentrated under reduced pressure to give the crude product (5.30 g) as brown oil. LC-MS (ESI) m z: 206 [M+H]⁺.

### Step 9: 2-(3-azabicyclo[3.1.0]hexan-3-yl)ethanol

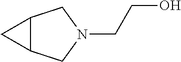

To a mixture of 3-azabicyclo[3.1.0]hexane (900 mg, 10.8 mmol) and 2-bromoethanol (1.61 g, 13.0 mmol) in MeCN (20 mL) was added K₂CO₃ (4.47 g, 32.4 mmol) and the reaction mixture was stirred at room temperature for 16 h. The mixture was filtered and the filtrate was evaporated under reduced pressure to give the crude product (1.20 g) as yellow oil. LC-MS (ESI) m z: 128 [M+H]⁺.

### Step 10: 3-(2-((5-bromopyridin-2-yl)methoxy)ethyl)-3-azabicyclo [3.1.0] hexane

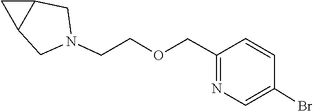 [SMILES: Brc1ccc(COCCN2CC3CC3C2)nc1]

To a solution of 2-(3-azabicyclo[3.1.0]hexan-3-yl)ethanol (1.20 g, 9.45 mmol) in dry THE (15 mL) was added NaH (756 mg, 18.9 mmol) slowly at 0° C. and the reaction mixture was stirred at 0° C. for 30 min. Then to the mixture was added a solution of 5-bromo-2-(chloromethyl)pyridine (1.94 g, 9.45 mmol) in THF (10 mL) dropwise, and the resulting reaction mixture was warmed to room temperature slowly and stirred at the temperature for 2 h. The mixture was poured into ice water (70 mL) and extracted with EtOAc (20 mL×2). The combined organic layers were washed with brine (30 mL), dried over anhydrous Na₂SO₄ and concentrated under reduced pressure. The residue was purified by chromatography on silica gel (DCM:MeOH=100:1 to 20:1) to give the desired product (950 mg, 26.7% yield for two steps) as brown oil. LC-MS (ESI) m z: 297 [M+H]⁺.

### Step 11: 8-(6-((2-(3-azabicyclo[3.1.0]hexan-3-yl)ethoxy)methyl)pyridin-3-yl)-1-isopropyl-3-methyl-1H-imidazo[4,5-c]cinnolin-2(3H)-one

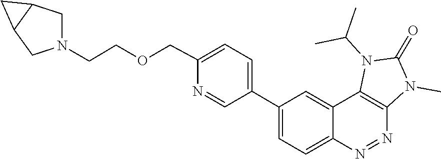 [SMILES: CC(C)n1c(=O)n(C)c2nnc3ccc(-c4ccc(COCCN5CC6CC6C5)nc4)cc3c21]

A mixture of 3-(2-((5-bromopyridin-2-yl)methoxy)ethyl)-3-azabicyclo[3.1.0]hexane (100 mg, 0.338 mmol), Bis(pinacolato)diboron (112 mg, 0.439 mmol), AcOK (99.4 mg, 1.01 mmol) and 1,1′-Bis(diphenylphosphino)ferrocene-palladium(II)dichloride dichloromethane complex (27.6 mg, 0.0338 mmol) in 1,4-dioxane (5 mL) was stirred at 100° C. for 2 h. After cooled to room temperature, to the reaction mixture was added water (1 mL), 8-bromo-1-isopropyl-3-methyl-1H-imidazo[4,5-c]cinnolin-2(3H)-one (108 mg, 0.338 mmol), K₂CO₃ (93.3 mg, 0.676 mmol) and 1,1′-Bis(diphenylphosphino)ferrocene-palladium(II)dichloride dichloromethane complex (27.6 mg, 0.0338 mmol) and the resulting reaction mixture was stirred at 100° C. for 2 h. The mixture was filtered and the filtrate was evaporated under reduced pressure. The residue was purified by flash chromatography (DCM:MeOH=9:1) to give the desired product (20.0 mg, 12.9% yield for two steps) as a brown solid. ¹H NMR (400 MHz, DMSO-d₆) δ 9.04 (d, J=2.3 Hz, 1H), 8.48-8.41 (m, 2H), 8.32 (dd, J=8.2, 2.4 Hz, 1H), 8.10 (dd, J=9.0, 1.8 Hz, 1H), 7.60 (d, J=8.0 Hz, 1H), 5.38-5.25 (m, 1H), 4.65 (s, 2H), 3.65-3.57 (m, 5H), 2.99 (d, J=8.6 Hz, 2H), 2.67 (t, J=5.8 Hz, 2H), 2.37-2.31 (m, 2H), 1.67 (d, J=6.6 Hz, 6H), 1.37-1.32 (m, 2H), 0.61-0.55 (m, 1H), 0.33-0.27 (m, 1H). LC-MS (ESI) m z: 459 [M+H]⁺.

The following compounds were prepared according to the above described methods using different starting materials.

| Ex | | | |
|---|---|---|---|
| # | Structure | Name | MS m/z |
| 2 | 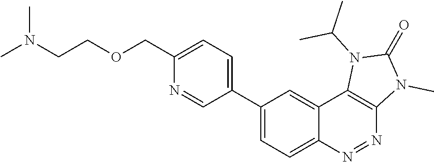 | 8-(6-((2- (dimethylamino)ethoxy)meth- yl)pyridin-3-yl)-1- isopropyl-3-methyl-1H- imidazo[4,5-c]cinnolin- 2(3H)-one | 421 [M + H]⁺ |
| 3 | 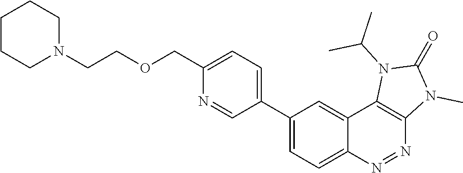 [SMILES: CC(C)n1c(=O)n(C)c2nnc3ccc(-c4ccc(COCCN5CCCCC5)nc4)cc3c21] | 1-isopropyl-3-methyl-8-(6- ((2-(piperidin-1- yl)ethoxy)methyl)pyridin- 3-yl)-1H-imidazo[4,5- c]cinnolin-2(3H)-one | 461 [M + H]⁺ |
| 4 | 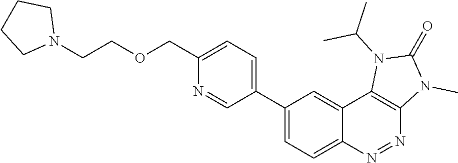 [SMILES: CC(C)n1c(=O)n(C)c2nnc3ccc(-c4ccc(COCCN5CCCC5)nc4)cc3c21] | 1-isopropyl-3-methyl-8-(6- ((2-(pyrrolidin-1- yl)ethoxy)methyl)pyridin- 3-yl)-1H-imidazo[4,5- c]cinnolin-2(3H)-one | 447 [M + H]⁺. |
| 5 | 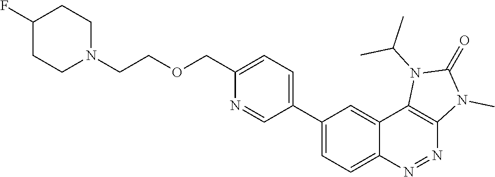 [SMILES: CC(C)n1c(=O)n(C)c2nnc3ccc(-c4ccc(COCCN5CCC(F)CC5)nc4)cc3c21] | 8-(6-((2-(4- fluropiperidin-1- yl)ethoxy)methyl)pyridin- 3-yl)-1-isopropyl-3- methyl-1H-imidazo[4,5- c]cinnolin-2(3H)-one | 479 [M + H]⁺ |
| 6 | 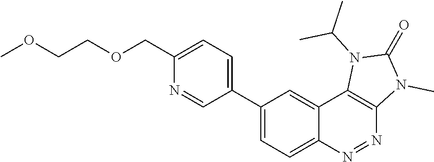 | 1-isopropyl-8-(6-((2- methoxyethoxy)methyl)py- ridin-3-yl)-3-methyl-1H- imidazo[4,5-c]cinnolin- 2(3H)-one | 408 [M + H]⁺ |
| 7 | 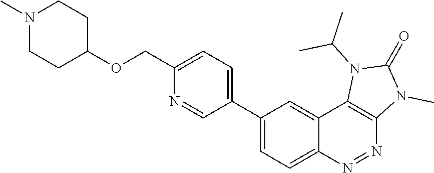 | 1-isopropyl-3-methyl-8-(6- (((1-methylpiperidin-4- yl)oxy)methyl)pyridin-3- yl)-1H-imidazo[4,5- c]cinnolin-2(3H)-one | 447 [M + H]⁺ |
| 8 | 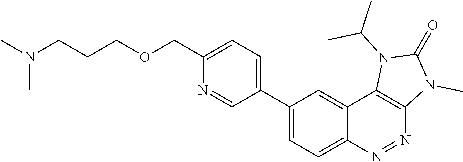 | 8-(6-((3- (dimethylamino)propoxy) methyl)pyridin-3-yl)-1- isopropyl-3-methyl-1H- imidazo[4,5-c]cinnolin- 2(3H)-one | 435 [M + H]⁺ |
| 9 | 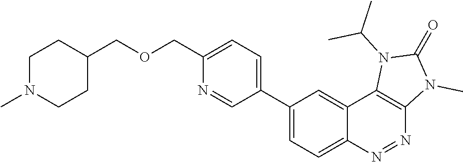 | 1-isopropyl-3-methyl-8-(6- (((1-methylpiperidin-4- yl)methoxy) methyl)pyridin-3-yl)-1H- imidazo[4,5-c]cinnolin- 2(3H)-one | 461 [M + H]⁺ |
| 10 | 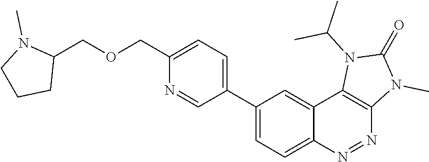 [SMILES: CC(C)n1c(=O)n(C)c2nnc3ccc(-c4ccc(COCC5CCCN5C)nc4)cc3c21] | 1-isopropyl-3-methyl-8-(6- (((1-methylpyrrolidin-2- yl)methoxy) methyl)pyridin-3-yl)-1H- imidazo[4,5-c]cinnolin- 2(3H)-one | 447 [M + H]⁺ |
| 11 | 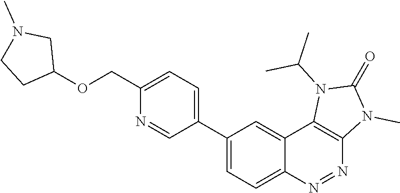 [SMILES: CC(C)n1c(=O)n(C)c2nnc3ccc(-c4ccc(COC5CCN(C)C5)nc4)cc3c21] | 1-isopropyl-3-methyl-8-(6- (((1-methylpyrrolidin-3- yl)oxy)methyl)pyridin-3- yl)-1H-imidazo[4,5- c]cinnolin-2(3H)-one | 433 [M + H]⁺ |
| 12 | 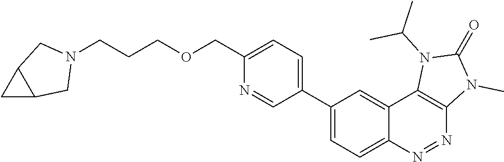 [SMILES: CC(C)n1c(=O)n(C)c2nnc3ccc(-c4ccc(COCCCN5CC6CC6C5)nc4)cc3c21] | 8-(6-((3-(3- azabicyclo[3.1.0]hexan-3- yl)propoxy)methyl)pyridin- 3-yl)-1-isopropyl-3- methyl-1H-imidazo[4,5- c]cinnolin-2(3H)-one | 473 [M + H]⁺ |
| 13 | 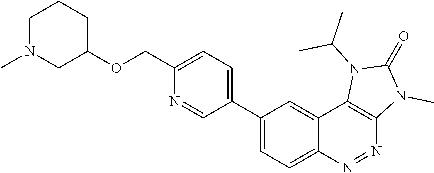 | 1-isopropyl-3-methyl-8-(6- (((1-methylpiperidin-3- yl)oxy)methyl)pyridin-3- yl)-1H-imidazo[4,5- c]cinnolin-2(3H)-one | 447 [M + H]⁺ |
| 14 | 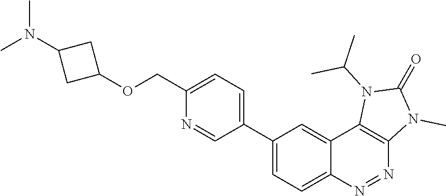 | 8-(6-((3- (dimethylamino)cyclobuto- xy)methyl)pyridin-3-yl)-1- isopropyl-3-methyl-1H- imidazo[4,5-c]cinnolin- 2(3H)-one | 447 [M + H]⁺ |
| 15 | 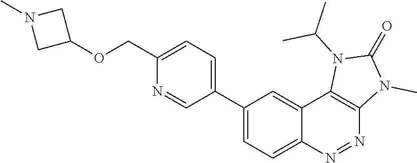 | 1-isopropyl-3-methyl-8-(6- (((1-methylazetidin-3- yl)oxy)methyl)pyridin-3- yl)-1H-imidazo[4,5- c]cinnolin-2(3H)-one | 419 [M + H]⁺ |
| 16 | 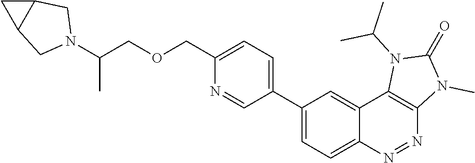 [SMILES: CC(COCc1ccc(-c2ccc3nnc4c(c3c2)n(C(C)C)c(=O)n4C)cn1)N1CC2CC2C1] | 8-(6-((2-(3- azabicyclo[3.1.0]hexan-3- yl)propoxy)methyl)pyridin- 3-yl)-1-isopropyl-3- methyl-1H-imidazo[4,5- c]cinnolin-2(3H)-one | 473 [M + H]⁺ |
| 17 | 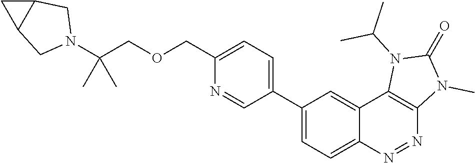 [SMILES: CC(C)n1c(=O)n(C)c2nnc3ccc(-c4ccc(COCC(C)(C)N5CC6CC6C5)nc4)cc3c21] | 8-(6-((2-(3- azabicyclo[3.1.0]hexan-3- yl)-2-methylpropoxy) methyl)pyridin-3-yl)-1- isopropyl-3-methyl-1H- imidazo[4,5-c]cinnolin- 2(3H)-one | 487 [M + H]⁺ |
| 18 | 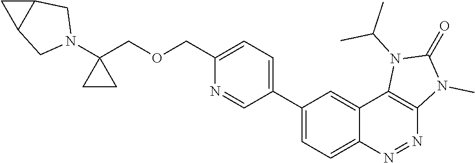 [SMILES: CC(C)n1c(=O)n(C)c2nnc3ccc(-c4ccc(COCC5(N6CC7CC7C6)CC5)nc4)cc3c21] | 8-(6-(((1-(3- azabicyclo[3.1.0]hexan-3- yl)cyclopropyl) methoxy)methyl)pyridin- 3-yl)-1-isopropyl-3- methyl-1H-imidazo[4,5- c]cinnolin-2(3H)-one | 485 [M + H]⁺ |
| 19 | 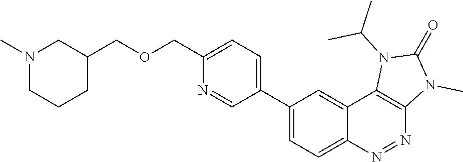 | 1-isopropyl-3-methyl-8-(6- (((1-methylpiperidin-3- yl)methoxy)methyl)pyridin- 3-yl)-1H-imidazo[4,5- c]cinnolin-2(3H)-one | 461 [M + H]⁺ |
| 20 | 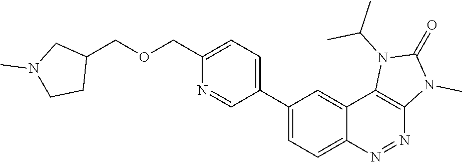 | 1-isopropyl-3-methyl-8-(6- (((1-methylpyrrolidin-3- yl)methoxy)methyl)pyridin- 3-yl)-1H-imidazo[4,5- c]cinnolin-2(3H)-one | 447 [M + H]⁺ |
| 21 | 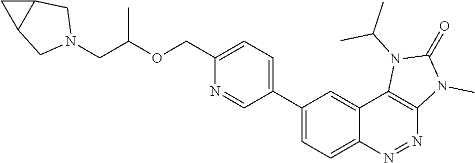 [SMILES: CC(CN1CC2CC2C1)OCc1ccc(-c2ccc3nnc4c(c3c2)n(C(C)C)c(=O)n4C)cn1] | 8-(6-(((1-(3- azabicyclo[3.1.0]hexan-3- yl)propan-2- yl)oxy)methyl)pyridin-3- yl)-1-isopropyl-3-methyl- 1H-imidazo[4,5- c]cinnolin-2(3H)-one | 473 [M + H]⁺ |
| 22 | 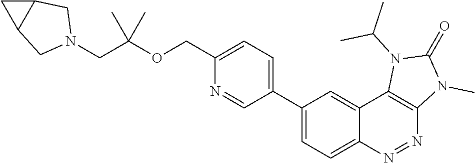 [SMILES: CC(C)n1c(=O)n(C)c2nnc3ccc(-c4ccc(COC(C)(C)CN5CC6CC6C5)nc4)cc3c21] | 8-(6-(((1-(3- azabicyclo[3.1.0]hexan-3- yl)-2-methylpropan-2- yl)oxy)methyl)pyridin-3- yl)-1-isopropyl-3-methyl- 1H-imidazo[4,5- c]cinnolin-2(3H)-one | 487 [M + H]⁺ |
| 23 | 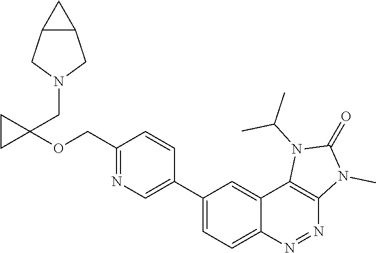 [SMILES: CC(C)n1c(=O)n(C)c2nnc3ccc(-c4ccc(COC5(CN6CC7CC7C6)CC5)nc4)cc3c21] | 8-(6-((1-(3- azabicyclo[3.1.0]hexan-3- ylmethyl)cyclopropoxy) methyl)pyridin-3-yl)-1- isopropyl-3-methyl-1H- imidazo[4,5-c]cinnolin- 2(3H)-one | 485 [M + H]⁺ |
| 24 | 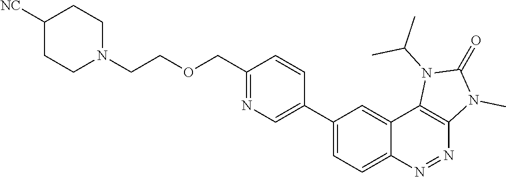 [SMILES: CC(C)n1c(=O)n(C)c2nnc3ccc(-c4ccc(COCCN5CCC(C#N)CC5)nc4)cc3c21] | 1-(2-((5-(1-isopropyl-3- methyl-2-oxo-2,3-dihydro- 1H-imidazo[4,5- c]cinnolin-8-yl)pyridin-2- yl)methoxy)ethyl)pyiperidine- 4-carbonitrile | 486 [M + H]⁺ |
| 25 | 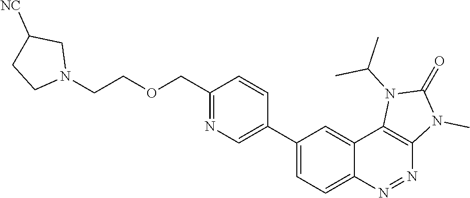 [SMILES: CC(C)n1c(=O)n(C)c2nnc3ccc(-c4ccc(COCCN5CCC(C#N)C5)nc4)cc3c21] | 1-(2-((R)-1-(5-(1- isopropyl-3-methyl-2-oxo- 2,3-dihydro-1H- imidazo[4,5-c]cinnolin-8- yl)pyridin-2- yl)ethoxy)ethyl)-3- methylpyrrolidine-3- carbonitrile | 472 [M + H]⁺ |

### Example 2

¹H NMR (600 MHz, DMSO-d₆) δ 9.05 (d, J=2.3 Hz, 1H), 8.47-8.41 (m, 2H), 8.33 (dd, J=8.1, 2.4 Hz, 1H), 8.10 (dd, J=9.0, 1.8 Hz, 1H), 7.62 (d, J=8.1 Hz, 1H), 5.36-5.27 (m, 1H), 4.66 (s, 2H), 3.66 (t, J=5.8 Hz, 2H), 3.60 (s, 3H), 2.53-2.52 (m, 2H), 2.20 (s, 6H), 1.67 (d, J=6.7 Hz, 6H).

### Example 3

¹H NMR (400 MHz, DMSO-d₆) δ 9.05 (d, J=2.4 Hz, 1H), 8.48-8.40 (m, 2H), 8.33 (dd, J=8.1, 2.4 Hz, 1H), 8.10 (dd, J=9.1, 1.7 Hz, 1H), 7.63 (d, J=8.1 Hz, 1H), 5.38-5.26 (m, 1H), 4.66 (s, 2H), 3.67 (t, J=5.9 Hz, 2H), 3.60 (s, 3H), 2.59 (t, J=5.8 Hz, 2H), 2.48-2.37 (m, 4H), 1.67 (d, J=6.7 Hz, 6H), 1.55-1.46 (m, 4H), 1.43-1.33 (m, 2H).

### Example 4

¹H NMR (400 MHz, DMSO-d₆) δ 9.04 (d, J=2.3 Hz, 1H), 8.47-8.40 (m, 2H), 8.33 (dd, J=8.1, 2.4 Hz, 1H), 8.10 (dd, J=9.1, 1.7 Hz, 1H), 7.62 (d, J=8.1 Hz, 1H), 5.36-5.26 (m, 1H), 4.66 (s, 2H), 3.67 (t, J=5.9 Hz, 2H), 3.60 (s, 3H), 3.43-3.41 (m, 4H), 2.69 (t, J=5.9 Hz, 2H), 1.73-1.61 (m, 10H).

### Example 5

¹H NMR (600 MHz, DMSO-d₆) δ 9.05 (d, J=2.4 Hz, 1H), 8.48-8.43 (m, 2H), 8.33 (dd, J=8.1, 2.4 Hz, 1H), 8.11 (dd, J=9.1, 1.8 Hz, 1H), 7.63 (d, J=8.1 Hz, 1H), 5.35-5.27 (m, 1H), 4.73-4.60 (m, 3H), 3.67 (t, J=5.8 Hz, 2H), 3.61 (s, 3H), 2.64-2.55 (m, 4H), 2.41-2.32 (m, 2H), 1.91-1.79 (m, 2H), 1.74-1.65 (m, 8H).

### Example 6

¹H NMR (400 MHz, DMSO-d₆) δ 9.01 (d, J=2.4 Hz, 1H), 8.45-8.35 (m, 2H), 8.30 (dd, J=8.2, 2.4 Hz, 1H), 8.07 (dd, J=9.0, 1.7 Hz, 1H), 7.59 (d, J=8.1 Hz, 1H), 5.35-5.21 (m, 1H), 4.65 (s, 2H), 3.71-3.63 (m, 2H), 3.57 (s, 3H), 3.55-3.50 (m, 2H), 3.27 (s, 3H), 1.65 (d, J=6.7 Hz, 6H).

### Example 7

¹H NMR (400 MHz, DMSO-d₆) δ 9.04 (d, J=2.3 Hz, 1H), 8.50-8.41 (m, 2H), 8.32 (dd, J=8.1, 2.4 Hz, 1H), 8.10 (dd, J=9.1, 1.8 Hz, 1H), 7.63 (d, J=8.2 Hz, 1H), 5.36-5.26 (m, 1H), 4.67 (s, 2H), 3.60 (s, 3H), 3.09-3.00 (m, 1H), 2.72-2.63 (m, 2H), 2.20 (s, 3H), 2.17-2.09 (m, 2H), 1.96-1.88 (m, 2H), 1.67 (d, J=6.7 Hz, 6H), 1.63-1.53 (m, 2H).

### Example 8

¹H NMR (400 MHz, DMSO-d₆) δ 9.05 (d, J=2.4 Hz, 1H), 8.48-8.41 (m, 2H), 8.33 (dd, J=8.2, 2.5 Hz, 1H), 8.11 (dd, J=9.1, 1.8 Hz, 1H), 7.61 (d, J=7.9 Hz, 1H), 5.37-5.27 (m, 1H), 4.63 (s, 2H), 3.60 (s, 3H), 3.58-3.56 (m, 2H), 2.32 (t, J=7.3 Hz, 2H), 2.13 (s, 6H), 1.78-1.70 (m, 2H), 1.66 (d, J=5.5 Hz, 6H).

### Example 9

¹H NMR (400 MHz, DMSO-d₆) δ 9.05 (d, J=2.4 Hz, 1H), 8.45 (dd, J=7.3, 5.4 Hz, 2H), 8.33 (dd, J=8.1, 2.4 Hz, 1H), 8.11 (dd, J=9.1, 1.8 Hz, 1H), 7.60 (d, J=8.1 Hz, 1H), 5.37-5.26 (m, 1H), 4.64 (s, 2H), 3.60 (s, 3H), 2.87-2.79 (m, 2H), 2.21 (s, 3H), 2.03-1.92 (m, 2H), 1.75-1.55 (m, 10H), 1.34-1.13 (m, 3H).

### Example 10

¹H NMR (400 MHz, DMSO-d₆) δ 9.02 (d, J=2.4 Hz, 1H), 8.46-8.38 (m, 2H), 8.31 (dd, J=8.1, 2.4 Hz, 1H), 8.08 (dd, J=9.1, 1.7 Hz, 1H), 7.59 (d, J=8.1 Hz, 1H), 5.35-5.22 (m, 1H), 4.64 (s, 2H), 3.59-3.56 (m, 4H), 3.47-3.43 (m, 1H), 2.96-2.88 (m, 1H), 2.44-2.39 (m, 1H), 2.32 (s, 3H), 2.17-2.09 (m, 1H), 1.93-1.83 (m, 1H), 1.69-1.58 (m, 8H), 1.56-1.47 (m, 1H).

### Example 11

¹H NMR (400 MHz, DMSO-d₆) δ 9.01 (d, J=2.4 Hz, 1H), 8.46-8.36 (m, 2H), 8.28 (dd, J=8.1, 2.4 Hz, 1H), 8.06 (dd, J=9.0, 1.7 Hz, 1H), 7.58 (d, J=8.2 Hz, 1H), 5.33-5.23 (m, 1H), 4.56 (s, 2H), 4.24-4.15 (m, 1H), 3.57 (s, 3H), 2.72-2.66 (m, 1H), 2.64-2.55 (m, 2H), 2.44-2.35 (m, 1H), 2.26 (s, 3H), 2.13-2.02 (m, 1H), 1.84-1.73 (m, 1H), 1.64 (d, J=6.7 Hz, 6H).

### Example 12

¹H NMR (400 MHz, DMSO-d₆) δ 8.99 (d, J=2.3 Hz, 1H), 8.43-8.35 (m, 2H), 8.26 (dd, J=8.1, 2.4 Hz, 1H), 8.05 (dd, J=9.0, 1.7 Hz, 1H), 7.54 (d, J=8.1 Hz, 1H), 5.33-5.20 (m, 1H), 4.57 (s, 2H), 3.55 (s, 3H), 3.50 (t, J=7.8, 4.8 Hz, 2H), 2.88 (d, J=8.5 Hz, 2H), 2.45-2.38 (m, 2H), 2.18 (d, J=8.5 Hz, 2H), 1.74-1.54 (m, 8H), 1.33-1.24 (m, 2H), 0.51 (m, J=3.8 Hz, 1H), 0.28-0.18 (m, 1H).

### Example 13

¹H NMR (400 MHz, DMSO-d₆) δ 9.05 (d, J=2.4 Hz, 1H), 8.50-8.41 (m, 2H), 8.32 (dd, J=8.2, 2.4 Hz, 1H), 8.11 (dd, J=9.1, 1.8 Hz, 1H), 7.63 (d, J=8.2 Hz, 1H), 5.40-5.25 (m, 1H), 4.70 (s, 2H), 3.60 (s, 3H), 3.58-3.52 (m, 1H), 3.00-2.91 (m, 1H), 2.61-2.55 (m, 1H), 2.22 (s, 3H), 2.04-1.88 (m, 3H), 1.74-1.58 (m, 7H), 1.50-1.39 (m, 1H), 1.29-1.18 (m, 1H).

### Example 14

¹H NMR (400 MHz, DMSO-d₆) δ 9.02 (d, J=2.4 Hz, 1H), 8.46-8.38 (m, 2H), 8.29 (dd, J=8.2, 2.4 Hz, 1H), 8.07 (d, J=9.0 Hz, 1H), 7.58 (d, J=8.1 Hz, 1H), 5.34-5.22 (m, 1H), 4.52 (s, 2H), 3.86-3.77 (m, 1H), 3.58 (s, 3H), 2.44-2.33 (m, 2H), 2.23-2.14 (m, 1H), 2.00 (s, 6H), 1.74-1.58 (m, 8H).

### Example 15

¹H NMR (400 MHz, DMSO-d₆) δ 9.00 (d, J=2.3 Hz, 1H), 8.44-8.35 (m, 2H), 8.27 (dd, J=8.3, 2.4 Hz, 1H), 8.05 (dd, J=9.1, 1.7 Hz, 1H), 7.56 (d, J=8.1 Hz, 1H), 5.34-5.20 (m, 1H), 4.52 (s, 2H), 4.21-4.11 (m, 1H), 3.56 (s, 3H), 3.28-3.24 (m, 2H), 2.84-2.76 (m, 2H), 2.20 (s, 3H), 1.63 (d, J=6.7 Hz, 6H).

### Example 16

¹H NMR (400 MHz, DMSO-d₆) δ 8.99 (d, J=2.3 Hz, 1H), 8.45-8.34 (m, 2H), 8.28 (dd, J=8.2, 2.3 Hz, 1H), 8.05 (d, J=9.1 Hz, 1H), 7.55 (d, J=8.1 Hz, 1H), 5.32-5.20 (m, 1H), 4.57 (s, 2H), 3.55 (s, 3H), 3.44-3.41 (m, 2H), 2.95-2.82 (m, 2H), 2.71-2.57 (m, 1H), 2.45-2.38 (m, 2H), 1.63 (d, J=6.7 Hz, 6H), 1.35-1.25 (m, 2H), 1.01 (d, J=6.5 Hz, 3H), 0.55-0.47 (m, 1H), 0.29-0.19 (m, 1H).

### Example 17

¹H NMR (400 MHz, DMSO-d₆) δ 9.02 (d, J=2.3 Hz, 1H), 8.46-8.38 (m, 2H), 8.32 (dd, J=8.1, 2.4 Hz, 1H), 8.08 (d, J=9.1 Hz, 1H), 7.58 (d, J=8.1 Hz, 1H), 5.35-5.23 (m, 1H), 4.63 (s, 2H), 3.58 (s, 3H), 3.49-3.42 (m, 2H), 2.79 (d, J=8.2 Hz, 2H), 2.65 (d, J=8.1 Hz, 2H), 1.65 (d, J=6.7 Hz, 6H), 1.35-1.24 (m, 2H), 1.01 (s, 6H), 0.53-0.46 (m, 1H), 0.29-0.19 (m, 1H).

### Example 18

¹H NMR (400 MHz, DMSO-d₆) δ 9.04 (s, 1H), 8.49-8.39 (m, 2H), 8.34 (d, J=8.0 Hz, 1H), 8.10 (d, J=8.8 Hz, 1H), 7.59 (d, J=7.7 Hz, 1H), 5.38-5.24 (m, 1H), 4.63 (s, 2H), 3.64-3.54 (m, 5H), 2.94 (d, J=7.9 Hz, 2H), 2.77 (d, J=8.2 Hz, 2H), 1.66 (d, J=6.4 Hz, 6H), 1.33-1.18 (m, 4H), 0.58-0.48 (m, 2H), 0.44-0.38 (m, 1H), 0.32-0.21 (m, 1H).

### Example 19

¹H NMR (400 MHz, DMSO-d₆) δ 9.02 (s, 1H), 8.47-8.37 (m, 2H), 8.31 (d, J=8.2 Hz, 1H), 8.08 (d, J=9.1 Hz, 1H), 7.57 (d, J=8.2 Hz, 1H), 5.36-5.20 (m, 1H), 4.60 (s, 2H), 3.58 (s, 3H), 2.82-2.72 (m, 1H), 2.65-2.55 (m, 1H), 2.12 (s, 3H), 1.96-1.76 (m, 3H), 1.73-1.53 (m, 8H), 1.52-1.35 (m, 2H), 1.02-0.85 (m, 2H).

### Example 20

¹H NMR (400 MHz, DMSO-d₆) δ 9.04 (dd, J=2.5, 0.8 Hz, 1H), 8.48-8.40 (m, 2H), 8.32 (dd, J=8.2, 2.4 Hz, 1H), 8.10 (dd, J=9.1, 1.7 Hz, 1H), 5.38-5.24 (m, 1H), 4.64 (s, 2H), 3.60 (s, 3H), 3.47-3.44 (m, 2H), 2.59-2.53 (m, 1H), 2.48-2.38 (m, 2H), 2.35-2.28 (m, 1H), 2.24 (s, 3H), 1.93-1.83 (m, 1H), 1.73-1.60 (m, 7H), 1.48-1.37 (m, 1H).

### Example 21

¹H NMR (400 MHz, DMSO-d₆) δ 9.00 (s, 1H), 8.46-8.35 (m, 2H), 8.27 (d, J=8.1 Hz, 1H), 8.07 (d, J=9.1 Hz, 1H), 7.60 (d, J=8.1 Hz, 1H), 5.34-5.22 (m, 1H), 4.66 (q, J=13.8 Hz, 2H), 3.69-3.61 (m, 1H), 3.57 (s, 3H), 3.01-2.87 (m, 2H), 2.63-2.51 (m, 1H), 2.44-2.37 (m, 1H), 2.36-2.21 (m, 2H), 1.64 (d, J=6.7 Hz, 6H), 1.34-1.24 (m, 2H), 1.12 (d, J=6.1 Hz, 3H), 0.59-0.52 (m, 1H), 0.34-0.19 (m, 1H).

### Example 22

¹H NMR (400 MHz, DMSO) δ 9.01 (d, J=2.4 Hz, 1H), 8.48-8.37 (m, 2H), 8.30 (dd, J=8.2, 2.5 Hz, 1H), 8.08 (dd, J=9.0, 1.7 Hz, 1H), 7.59 (d, J=8.2 Hz, 1H), 5.38-5.24 (m, 1H), 4.58 (s, 2H), 3.59 (s, 3H), 3.06 (d, J=8.6 Hz, 2H), 2.53 (s, 2H), 2.43 (d, J=8.7 Hz, 2H), 1.67 (d, J=6.7 Hz, 6H), 1.38-1.25 (m, 2H), 1.19 (s, 6H), 0.62-0.53 (m, 1H), 0.34-0.23 (m, 1H).

### Example 23

¹H NMR (400 MHz, DMSO-d₆) δ 8.95 (s, 1H), 8.42-8.34 (m, 2H), 8.23 (dd, J=8.1, 2.3 Hz, 1H), 8.04 (d, J=9.1 Hz, 1H), 7.52 (d, J=8.2 Hz, 1H), 5.32-5.17 (m, 1H), 4.67 (s, 2H), 3.56 (s, 3H), 3.03 (d, J=8.6 Hz, 2H), 2.63 (s, 2H), 2.37-2.25 (m, 2H), 1.63 (d, J=6.7 Hz, 6H), 1.35-1.24 (m, 2H), 0.85-0.75 (m, 2H), 0.56-0.44 (m, 3H), 0.30-0.21 (m, 1H).

### Example 24

¹H NMR (400 MHz, DMSO-d₆) δ 9.05 (d, J=2.1 Hz, 1H), 8.48-8.40 (m, 2H), 8.33 (dd, J=8.1, 2.4 Hz, 1H), 8.10 (dd, J=9.1, 1.6 Hz, 1H), 7.62 (d, J=8.2 Hz, 1H), 5.39-5.25 (m, 1H), 4.66 (s, 2H), 3.66 (t, J=5.7 Hz, 2H), 3.60 (s, 3H), 2.90-2.80 (m, 1H), 2.69-2.56 (m, 4H), 2.43-2.29 (m, 2H), 1.90-1.81 (m, 2H), 1.75-1.69 (m, 2H), 1.67 (d, J=6.7 Hz, 6H).

### Example 25

¹H NMR (400 MHz, DMSO-d₆) δ 9.05 (d, J=2.1 Hz, 1H), 8.49-8.40 (m, 2H), 8.32 (dd, J=8.1, 2.3 Hz, 1H), 8.09 (dd, J=9.1, 1.5 Hz, 1H), 7.63 (d, J=8.2 Hz, 1H), 5.36-5.26 (m, 1H), 4.67 (s, 2H), 3.68 (t, J=5.6 Hz, 2H), 3.60 (s, 3H), 3.30-3.20 (m, 1H), 2.85-2.66 (m, 5H), 2.56-2.52 (m, 1H), 2.25-2.12 (m, 1H), 1.99-1.88 (m, 1H), 1.67 (d, J=6.7 Hz, 6H).

### Example 26

### 8-(6-((2-(5-azaspiro[2.4]heptan-5-yl)ethoxy)methyl)pyridin-3-yl)-1-isopropyl-3-methyl-1H-imidazo[4,5-c]cinnolin-2(3H)-one

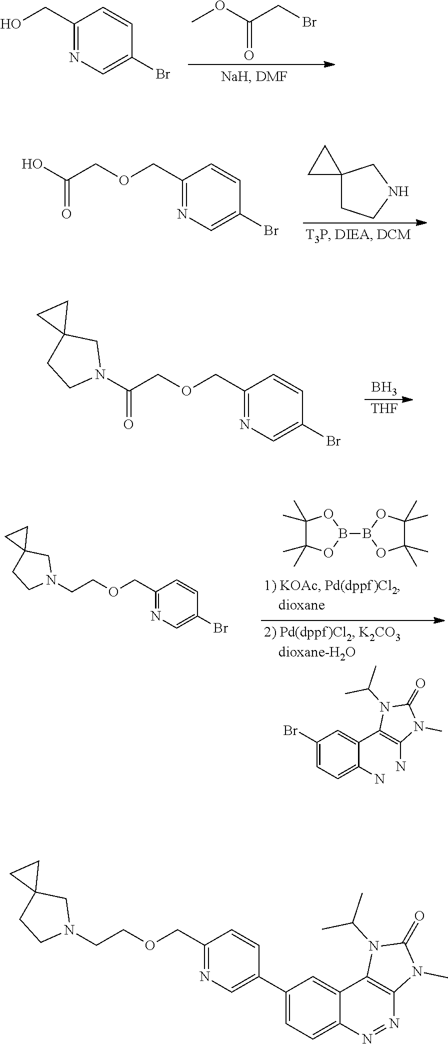

### Step 1: 2-((5-bromopyridin-2-yl)methoxy)acetic acid

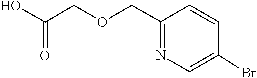 [SMILES: O=C(O)COCc1ccc(Br)cn1]

To a solution of (5-bromopyridin-2-yl)methanol (5.00 g, 27.0 mmol) in dry DMF (50 mL) was added NaH slowly at 0° C. and the reaction mixture was stirred at 0° C. for 30 min. Then to the mixture was added methyl 2-bromoacetate (3.60 mL, 35.0 mmol) dropwise, the resulting reaction mixture was warmed to room temperature slowly and stirred at room temperature for 3 h. The mixture was poured into ice 1 M NaOH solution (70 mL) and stirred at room temperature for 1 h. The mixture was extracted with EtOAc (10 mL×2) and the water layer was adjusted with 1 M HCl solution to pH˜4. It was extracted with EtOAc (100 mL×3), the organic layers were washed with 5% LiCl solution (20 mL) and brine (20 mL), dried over anhydrous Na₂SO₄ and concentrated under reduced pressure to give crude product that was triturated with MTBE (50 mL) at room temperature for 20 min and filtered. The filtered cake was dried under vacuum for 3 h to give the desired product (4.30 g, 65% yield) as a white solid.

### Step 2: 2-((5-bromopyridin-2-yl)methoxy)-1-(5-azaspiro[2.4]heptan-5-yl)ethan-1-one

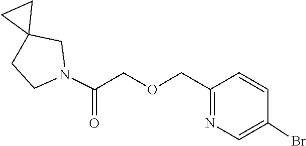 [SMILES: O=C(COCc1ccc(Br)cn1)N1CCC2(CC2)C1]

A mixture of 2-((5-bromopyridin-2-yl)methoxy)acetic acid (500 mg, 2.04 mmol) and 5-Azaspiro[2.4]heptane hydrochloride (271 mg, 2.04 mmol) in DCM (10 mL) was added 50% T₃P in EtOAc (2.60 g, 4.08 mmol) followed by DIEA (1.0 mL, 6.12 mol) at 0° C. After stirred at room temperature for 16 h, the mixture was poured into ice sat. NaHCO₃ solution (20 mL) and extracted with DCM (20 mL×2). The organic layer was washed with sat. NH₄Cl solution (20 mL) and brine (20 mL), dried over anhydrous Na₂SO₄ and filtered. The filtrate was concentrated under reduced pressure to give crude product (600 mg) as brown oil. LC-MS (ESI) m z: 325 [M+H]⁺.

### Step 3: 5-(2-((5-bromopyridin-2-yl)methoxy)ethyl)-5-azaspiro[2.4]heptane

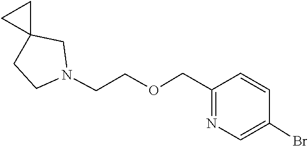 [SMILES: Brc1ccc(COCCN2CCC3(CC3)C2)nc1]

To a solution of 2-((5-bromopyridin-2-yl)methoxy)-1-(5-azaspiro[2.4]heptan-5-yl)ethan-1-one (600 mg, 1.85 mmol) in dry THE was added 1 M BH₃·THF solution (9.25 mL, 9.25 mmol) dropwise at 0° C. and the reaction mixture was stirred at room temperature for 2 h. The mixture was quenched with MeOH (10 mL) and evaporated under reduced pressure. The residue was dissolved in EtOH (20 mL) and stirred at 90° C. for 3 h. The mixture was evaporated under reduced pressure and the residue was purified by chromatography on silica gel (DCM:MeOH=100:1 to 10:1) to give the desired product (329 mg, 52.0% yield for two steps) as light brown oil. LC-MS (ESI) m/z: 311 [M+H]⁺.

### Step 4: 8-(6-((2-(5-azaspiro[2.4]heptan-5-yl)ethoxy)methyl)pyridin-3-yl)-1-isopropyl-3-methyl-1H-imidazo[4,5-c]cinnolin-2(3H)-one

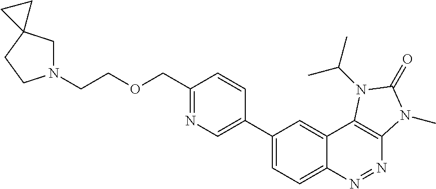 [SMILES: CC(C)n1c(=O)n(C)c2nnc3ccc(-c4ccc(COCCN5CCC6(CC6)C5)nc4)cc3c21]

The crude product was prepared in a similar fashion to Example 1, which was purified by flash chromatograph (DCM:MeOH=9:1) to give the desired product (50.0 mg, 21.9% yield for two steps) as a light brown solid. ¹H NMR (400 MHz, DMSO-d₆) δ 9.05 (d, J=2.0 Hz, 1H), 8.49-8.41 (m, 2H), 8.33 (dd, J=8.1, 2.3 Hz, 1H), 8.11 (dd, J=9.1, 1.5 Hz, 1H), 7.63 (d, J=8.2 Hz, 1H), 5.37-5.27 (m, 1H), 4.67 (s, 2H), 3.67 (t, J=5.9 Hz, 2H), 3.60 (s, 3H), 2.77-2.67 (m, 4H), 2.53 (s, 2H), 1.73 (t, J=6.9 Hz, 2H), 1.67 (d, J=6.7 Hz, 6H), 0.56-0.45 (m, 4H). LC-MS (ESI) m z: 473 [M+H]⁺.

The following compounds were prepared according to the above described methods using different starting materials.

| | | | MS |
|---|---|---|---|
| Ex# | Structure | Name | m/z |
| 27 | 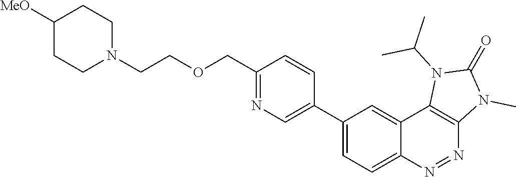 [SMILES: COC1CCN(CCOCc2ccc(-c3ccc4nnc5c(c4c3)n(C(C)C)c(=O)n5C)cn2)CC1] | 1-isopropyl-8-(6-((2-(4- methoxypiperidin-1- yl)ethoxy)methyl)pyridin-3- yl)-3-methyl-1H-imidazo[4,5- c]cinnolin-2(3H)-one | 491 [M + H]⁺ |
| 28 | 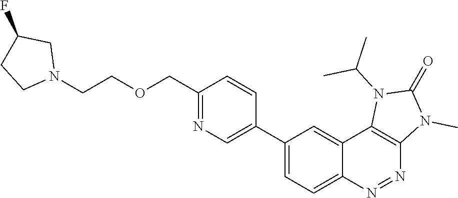 [SMILES: CC(C)n1c(=O)n(C)c2nnc3ccc(-c4ccc(COCCN5CC[C@@H](F)C5)nc4)cc3c21] | (R)-8-(6-((2-(3- fluoropyrrolidin-1- yl)ethoxy)methyl)pyridin-3- yl)-1-isopropyl-3-methyl-1H- imidazo[4,5-c]cinnolin- 2(3H)-one | 465 [M + H]⁺ |
| 29 | 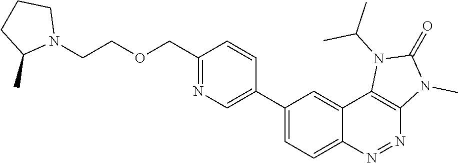 [SMILES: CC(C)n1c(=O)n(C)c2nnc3ccc(-c4ccc(COCCN5CCC[C@@H]5C)nc4)cc3c21] | (S)-1-isopropyl-3-methyl-8- (6-((2-(2-methylpyrrolidin-1- yl)ethoxy)methyl)pyridin-3- yl)-1H-imidazo[4,5- c]cinnolin-2(3H)-one | 461 [M + H]⁺ |
| 30 | 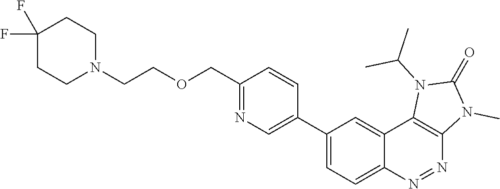 [SMILES: CC(C)n1c(=O)n(C)c2nnc3ccc(-c4ccc(COCCN5CCC(F)(F)CC5)nc4)cc3c21] | 8-(6-((2-(4,4- difluoropiperidin-1- yl)ethoxy)methyl)pyridin-3- yl)-1-isopropyl-3-methyl-1H- imidazo[4,5-c]cinnolin- 2(3H)-one | 497 [M + H]⁺ |
| 31 | 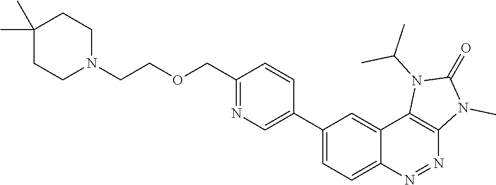 | 8-(6-((2-(4,4- dimethylpiperidin-1- yl)ethoxy)methyl)pyridin-3- yl)-1-isopropyl-3-methyl-1H- imidazo[4,5-c]cinnolin- 2(3H)-one | 489 [M + H]⁺ |
| 32 | 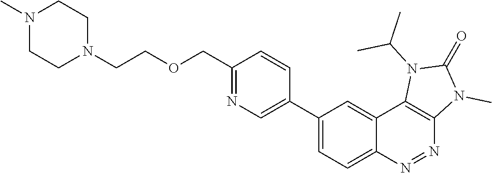 | 1-isopropyl-3-methyl-8-(6- ((2-(4-methylpiperazin-1- yl)ethoxy)methyl)pyridin-3- yl)-1H-imidazo[4,5- c]cinnolin-2(3H)-one | 476 [M + H]⁺ |
| 33 | 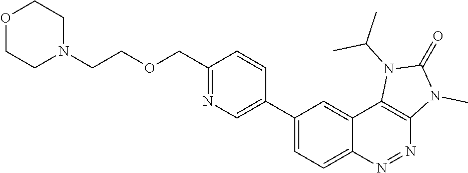 [SMILES: CC(C)n1c(=O)n(C)c2nnc3ccc(-c4ccc(COCCN5CCOCC5)nc4)cc3c21] | 1-isopropyl-3-methyl-8-(6- ((2- morpholinoethoxy)methyl) pyridin-3-yl)-1H- imidazo[4,5-c]cinnolin- 2(3H)-one | 463 [M + H]⁺ |
| 34 | 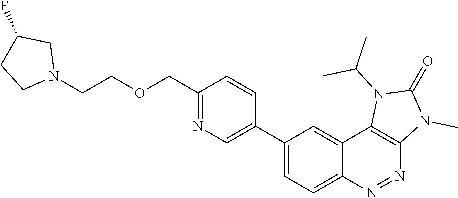 [SMILES: CC(C)n1c(=O)n(C)c2nnc3ccc(-c4ccc(COCCN5CC[C@H](F)C5)nc4)cc3c21] | (S)-8-(6-((2-(3- fluoropyrrolidin-1- yl)ethoxy)methyl)pyridin-3- yl)-1-isopropyl-3-methyl-1H- imidazo[4,5-c]cinnolin- 2(3H)-one | 465 [M + H]⁺ |
| 35 | 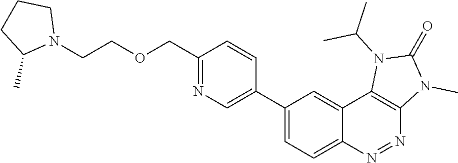 [SMILES: CC(C)n1c(=O)n(C)c2nnc3ccc(-c4ccc(COCCN5CCC[C@H]5C)nc4)cc3c21] | (R)-1-isopropyl-3-methyl-8- (6-((2-(2-methylpyrrolidin-1- yl)ethoxy)methyl)pyridin-3- yl)-1H-imidazo[4,5- c]cinnolin-2(3H)-one | 461 [M + H]⁺ |
| 36 | 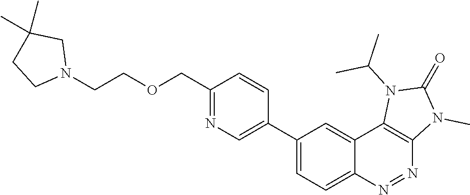 | 8-(6-((2-(3,3- dimethylpyrrolidin-1- yl)ethoxy)methyl)pyridin-3- yl)-1-isopropyl-3-methyl-1H- imidazo[4,5-c]cinnolin- 2(3H)-one | 475 [M + H]⁺ |
| 37 | 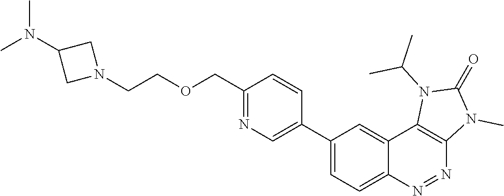 | 8-(6-((2-(3- (dimethylamino)azetidin-1- yl)ethoxy)methyl)pyridin-3- yl)-1-isopropyl-3-methyl-1H- imidazo[4,5-c]cinnolin- 2(3H)-one | 476 [M + H]⁺ |
| 38 | 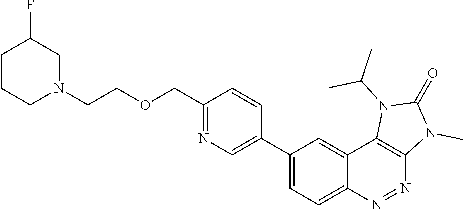 [SMILES: CC(C)n1c(=O)n(C)c2nnc3ccc(-c4ccc(COCCN5CCCC(F)C5)nc4)cc3c21] | 8-(6-((2-(3-fluoropiperidin-1- yl)ethoxy)methyl)pyridin-3- yl)-1-isopropyl-3-methyl-1H- imidazo[4,5-c]cinnolin- 2(3H)-one | 479 [M + H]⁺ |
| 39 | 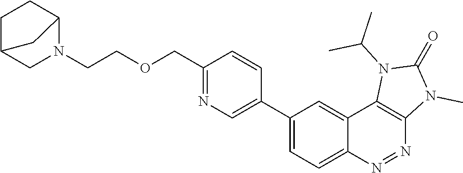 [SMILES: CC(C)n1c(=O)n(C)c2nnc3ccc(-c4ccc(COCCN5CC6CCC5C6)nc4)cc3c21] | 8-(6-((2-(2- azabicyclo[2.2.1]heptan-2- yl)ethoxy)methyl)pyridin-3- yl)-1-isopropyl-3-methyl-1H- imidazo[4,5-c]cinnolin- 2(3H)-one | 473 [M + H]⁺ |
| 40 | 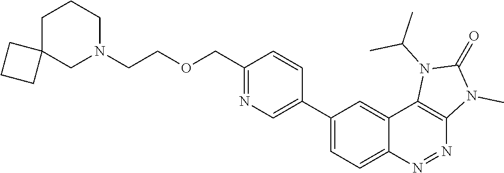 [SMILES: CC(C)n1c(=O)n(C)c2nnc3ccc(-c4ccc(COCCN5CCCC6(CCC6)C5)nc4)cc3c21] | 8-(6-((2-(6- azaspiro[3.5]nonan-6- yl)ethoxy)methyl)pyridin-3- yl)-1-isopropyl-3-methyl-1H- imidazo[4,5-c]cinnolin- 2(3H)-one | 501 [M + H]⁺ |
| 41 | 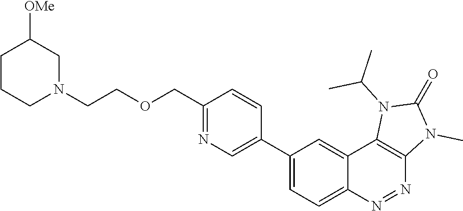 [SMILES: COC1CCCN(CCOCc2ccc(-c3ccc4nnc5c(c4c3)n(C(C)C)c(=O)n5C)cn2)C1] | 1-isopropyl-8-(6-((2-(3- methoxypiperidin-1- yl)ethoxy)methyl)pyridin-3- yl)-3-methyl-1H-imidazo[4,5- c]cinnolin-2(3H)-one | 491 [M + H]⁺ |
| 42 | 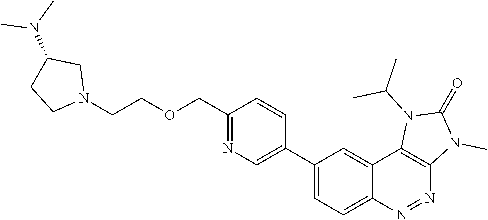 | (S)-8-(6-((2-(3- (dimethylamino)pyrrolidin-1- yl)ethoxy)methyl)pyridin-3- yl)-1-isopropyl-3-methyl-1H- imidazo[4,5-c]cinnolin- 2(3H)-one | 490 [M + H]⁺ |
| 43 | 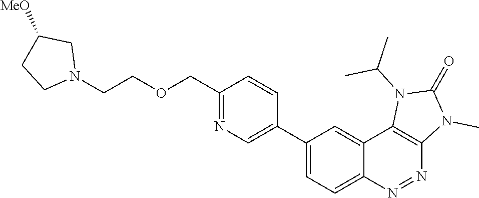 [SMILES: CO[C@H]1CCN(CCOCc2ccc(-c3ccc4nnc5c(c4c3)n(C(C)C)c(=O)n5C)cn2)C1] | (S)-1-isopropyl-8-(6-((2-(3- methoxypyrrolidin-1- yl)ethoxy)methyl)pyridin-3- yl)-3-methyl-1H-imidazo[4,5- c]cinnolin-2(3H)-one | 477 [M + H]⁺ |
| 44 | 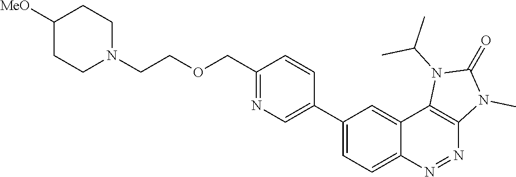 [SMILES: COC1CCN(CCOCc2ccc(-c3ccc4nnc5c(c4c3)n(C(C)C)c(=O)n5C)cn2)CC1] | 1-isopropyl-8-(6-((2-(4- methoxypiperidin-1- yl)ethoxy)methyl)pyridin-3- yl)-3-methyl-1H-imidazo[4,5- c]cinnolin-2(3H)-one | 491 [M + H]⁺ |
| 45 | 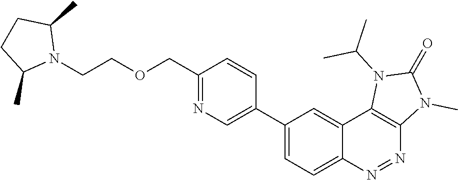 [SMILES: CC(C)n1c(=O)n(C)c2nnc3ccc(-c4ccc(COCCN5[C@H](C)CC[C@@H]5C)nc4)cc3c21] | 8-(6-((2-((2R,5S)-2,5- dimethylpyrrolidin-1- yl)ethoxy)methyl)pyridin-3- yl)-1-isopropyl-3-methyl-1H- imidazo[4,5-c]cinnolin- 2(3H)-one | 475 [M + H]⁺ |
| 46 | 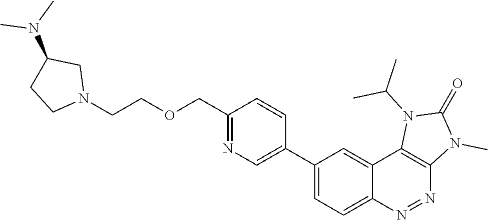 | (R)-8-(6-((2-(3- (dimethylamino)pyrrolidin-1- yl)ethoxy)methyl)pyridin-3- yl)-1-isopropyl-3-methyl-1H- imidazo[4,5-c]cinnolin- 2(3H)-one | 490 [M + H]⁺ |
| 47 | 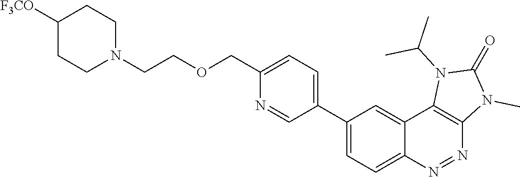 [SMILES: CC(C)n1c(=O)n(C)c2nnc3ccc(-c4ccc(COCCN5CCC(OC(F)(F)F)CC5)nc4)cc3c21] | 1-isopropyl-3-methyl-8-(6- ((2-(4- (trifluoromethoxy)piperidin- 1-yl)ethoxy)methyl)pyridin- 3-yl)-1H-imidazo[4,5- c]cinnolin-2(3H)-one | 545 [M + H]⁺ |
| 48 | 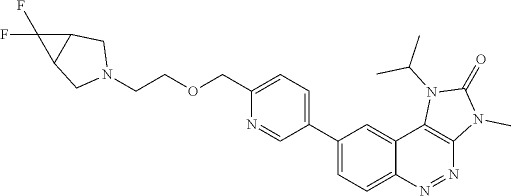 [SMILES: CC(C)n1c(=O)n(C)c2nnc3ccc(-c4ccc(COCCN5CC6C(C5)C6(F)F)nc4)cc3c21] | 8-(6-((2-(6,6-difluoro-3- azabicyclo[3.1.0]hexan-3- yl)ethoxy)methyl)pyridin-3- yl)-1-isopropyl-3-methyl-1H- imidazo[4,5-c]cinnolin- 2(3H)-one | 495 [M + H]⁺ |
| 49 | 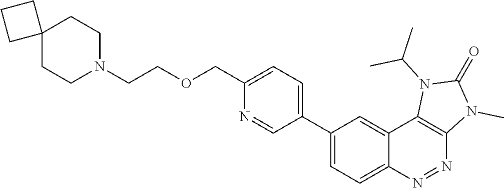 [SMILES: CC(C)n1c(=O)n(C)c2nnc3ccc(-c4ccc(COCCN5CCC6(CCC6)CC5)nc4)cc3c21] | 8-(6-((2-(7- azaspiro[3.5]nonan-7- yl)ethoxy)methyl)pyridin-3- yl)-1-isopropyl-3-methyl-1H- imidazo[4,5-c]cinnolin- 2(3H)-one | 501 [M + H]⁺ |
| 50 | 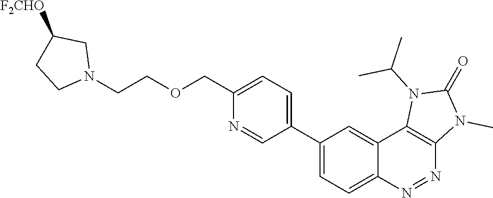 | (R)-8-(6-((2-(3- (difluoromethoxy)pyrrolidin- 1-yl)ethoxy)methyl)pyridin- 3-yl)-1-isopropyl-3-methyl- 1H-imidazo[4,5-c]cinnolin- 2(3H)-one | 513 [M + H]⁺ |
| 51 | 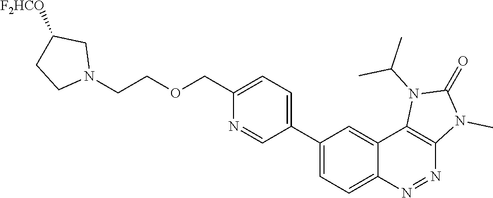 [SMILES: CC(C)n1c(=O)n(C)c2nnc3ccc(-c4ccc(COCCN5CC[C@H](OC(F)F)C5)nc4)cc3c21] | (S)-8-(6-((2-(3- (difluoromethoxy)pyrrolidin- 1-yl)ethoxy)methyl)pyridin- 3-yl)-1-isopropyl-3-methyl- 1H-imidazo[4,5-c]cinnolin- 2(3H)-one | 513 [M + H]⁺ |
| 52 | 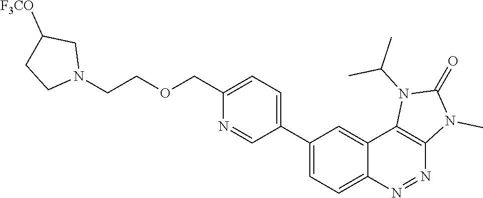 [SMILES: CC(C)n1c(=O)n(C)c2nnc3ccc(-c4ccc(COCCN5CCC(OC(F)(F)F)C5)nc4)cc3c21] | 1-isopropyl-3-methyl-8-(6- ((2-(3- (trifluoromethoxy)pyrrolidin- 1-yl)ethoxy)methyl)pyridin- 3-yl)-1,3-dihydro-2H- imidazo[4,5-c]cinnolin-2-one | 531 [M + H]⁺ |

### Example 27

¹H NMR (400 MHz, DMSO-d₆) δ 9.04 (s, 1H), 8.48-8.39 (m, 2H), 8.32 (d, J=8.1 Hz, 1H), 8.09 (d, J=9.1 Hz, 1H), 7.61 (d, J=8.0 Hz, 1H), 5.36-5.26 (m, 1H), 4.65 (s, 2H), 3.90-3.80 (m, 1H), 3.66 (t, J=5.5 Hz, 2H), 3.60 (s, 3H), 3.16 (s, 3H), 2.77-2.52 (m, 6H), 2.01-1.90 (m, 1H), 1.73-1.59 (m, 7H).

### Example 28

¹H NMR (400 MHz, DMSO-d₆) δ 9.04 (d, J=2.3 Hz, 1H), 8.47-8.39 (m, 2H), 8.32 (dd, J=8.2, 2.4 Hz, 1H), 8.09 (dd, J=9.1, 1.8 Hz, 1H), 7.62 (d, J=8.1 Hz, 1H), 5.37-5.29 (m, 1H), 5.28-5.07 (m, 1H), 4.66 (s, 2H), 3.67 (t, J=5.8 Hz, 2H), 3.59 (s, 3H), 2.94-2.81 (m, 2H), 2.75-2.57 (m, 3H), 2.40-2.30 (m, 1H), 2.19-2.01 (m, 1H), 1.94-1.75 (m, 1H), 1.66 (d, J=6.8 Hz, 6H).

### Example 29

¹H NMR (400 MHz, DMSO-d₆) δ 9.03 (d, J=2.2 Hz, 1H), 8.47-8.38 (m, 2H), 8.31 (dd, J=8.1, 2.3 Hz, 1H), 8.08 (d, J=9.0 Hz, 1H), 7.60 (d, J=8.1 Hz, 1H), 5.35-5.24 (m, 1H), 4.65 (s, 2H), 3.65 (t, J=6.0 Hz, 2H), 3.59 (s, 3H), 3.13-3.06 (m, 1H), 3.04-2.95 (m, 1H), 2.36-2.26 (m, 2H), 2.19-2.09 (m, 1H), 1.89-1.79 (m, 1H), 1.73-1.57 (m, 8H), 1.33-1.19 (m, 1H), 1.02 (d, J=6.0 Hz, 3H).

### Example 30

¹H NMR (400 MHz, DMSO-d₆) δ 9.03 (s, 1H), 8.48-8.38 (m, 2H), 8.31 (d, J=7.6 Hz, 1H), 8.08 (d, J=9.0 Hz, 1H), 7.60 (d, J=7.8 Hz, 1H), 5.35-5.23 (m, 1H), 4.65 (s, 2H), 3.73-3.62 (m, 2H), 3.58 (s, 3H), 2.69-2.61 (m, 2H), 2.61-2.52 (m, 4H), 2.01-1.85 (m, 4H), 1.65 (d, J=6.3 Hz, 6H).

### Example 31

¹H NMR (400 MHz, DMSO-d₆) δ 9.03 (d, J=2.0 Hz, 1H), 8.46-8.38 (m, 2H), 8.31 (dd, J=8.1, 2.3 Hz, 1H), 8.08 (dd, J=9.1, 1.6 Hz, 1H), 7.61 (d, J=8.1 Hz, 1H), 5.38-5.25 (m, 1H), 4.65 (s, 2H), 3.66 (t, J=5.9 Hz, 2H), 3.59 (s, 3H), 2.57 (t, J=5.9 Hz, 2H), 2.46-2.34 (m, 4H), 1.67 (d, J=6.8 Hz, 6H), 1.35-1.27 (m, 4H), 0.88 (s, 6H).

### Example 32

¹H NMR (400 MHz, DMSO-d₆) δ 9.04 (d, J=2.1 Hz, 1H), 8.48-8.40 (m, 2H), 8.32 (dd, J=8.1, 2.4 Hz, 1H), 8.09 (dd, J=9.1, 1.6 Hz, 1H), 7.61 (d, J=8.1 Hz, 1H), 5.37-5.23 (m, 1H), 4.65 (s, 2H), 3.66 (t, J=5.8 Hz, 2H), 3.59 (s, 3H), 2.56 (t, J=5.8 Hz, 2H), 2.48-2.18 (m, 8H), 2.14 (s, 3H), 1.67 (d, J=6.8 Hz, 6H).

### Example 33

¹H NMR (400 MHz, DMSO-d₆) δ 9.04 (d, J=2.3 Hz, 1H), 8.47-8.39 (m, 2H), 8.32 (dd, J=8.1, 2.4 Hz, 1H), 8.09 (dd, J=9.1, 1.7 Hz, 1H), 7.62 (d, J=8.1 Hz, 1H), 5.37-5.26 (m, 1H), 4.66 (s, 2H), 3.69 (t, J=5.7 Hz, 2H), 3.62-3.51 (m, 7H), 2.58 (t, J=5.8 Hz, 2H), 2.47-2.39 (m, 4H), 1.67 (d, J=6.7 Hz, 6H).

### Example 34

¹H NMR (400 MHz, DMSO-d₆) δ 9.03 (s, 1H), 8.48-8.38 (m, 2H), 8.31 (d, J=8.1 Hz, 1H), 8.08 (d, J=9.1 Hz, 1H), 7.61 (d, J=8.2 Hz, 1H), 5.36-5.19 (m, 1H), 5.25-5.05 (m, 1H), 4.65 (s, 2H), 3.66 (t, J=5.9 Hz, 2H), 3.58 (s, 3H), 2.94-2.80 (m, 2H), 2.70 (t, J=5.9 Hz, 2H), 2.64-2.53 (m, 1H), 2.40-2.31 (m, 1H), 2.18-2.03 (m, 1H), 1.92-1.76 (m, 1H), 1.65 (d, J=6.7 Hz, 6H).

### Example 35

¹H NMR (400 MHz, DMSO-d₆) δ 9.04 (s, 1H), 8.48-8.40 (m, 2H), 8.32 (d, J=7.9 Hz, 1H), 8.09 (d, J=8.9 Hz, 1H), 7.61 (d, J=8.1 Hz, 1H), 5.36-5.26 (m, 1H), 4.66 (s, 2H), 3.67 (t, J=5.5 Hz, 2H), 3.59 (s, 3H), 3.17-2.99 (m, 1H), 2.65-2.53 (m, 1H), 2.42-2.14 (m, 2H), 1.93-1.82 (m, 1H), 1.71-1.57 (m, 9H), 1.33-1.20 (m, 1H), 1.05 (d, J=5.6 Hz, 3H).

### Example 36

¹H NMR (400 MHz, DMSO-d₆) δ 9.02 (s, 1H), 8.45-8.37 (m, 2H), 8.29 (d, J=8.2 Hz, 1H), 8.07 (d, J=8.9 Hz, 1H), 7.60 (d, J=8.1 Hz, 1H), 5.35-5.23 (m, 1H), 4.64 (s, 2H), 3.62 (t, J=5.9 Hz, 2H), 3.58 (s, 3H), 2.67-2.56 (m, 4H), 2.31 (s, 2H), 1.65 (d, J=6.7 Hz, 6H), 1.48 (t, J=7.0 Hz, 2H), 1.02 (s, 6H).

### Example 37

¹H NMR (400 MHz, DMSO-d₆) δ 9.03 (d, J=2.3 Hz, 1H), 8.47-8.40 (m, 2H), 8.32 (dd, J=8.2, 2.3 Hz, 1H), 8.08 (d, J=9.1 Hz, 1H), 7.60 (d, J=8.1 Hz, 1H), 5.36-5.24 (m, 1H), 4.62 (s, 2H), 3.59 (s, 3H), 3.52 (t, J=5.7 Hz, 2H), 3.46-3.42 (m, 2H), 2.83-2.71 (m, 3H), 2.62 (t, J=5.7 Hz, 2H), 1.98 (s, 5H), 1.66 (d, J=6.9, 1.8 Hz, 6H).

### Example 38

¹H NMR (400 MHz, DMSO-d₆) δ 9.03 (s, 1H), 8.46-8.38 (m, 2H), 8.30 (d, J=8.2 Hz, 1H), 8.08 (d, J=9.3 Hz, 1H), 7.61 (d, J=8.1 Hz, 1H), 5.36-5.23 (m, 1H), 4.70-4.49 (m, 3H), 3.66 (t, J=5.6 Hz, 2H), 3.59 (s, 3H), 2.86-2.73 (m, 1H), 2.61 (t, J=5.6 Hz, 2H), 2.45-2.36 (m, 1H), 2.34-2.24 (m, 1H), 1.89-1.69 (m, 2H), 1.66 (d, J=6.7 Hz, 6H), 1.54-1.34 (m, 3H).

### Example 39

¹H NMR (400 MHz, DMSO-d₆) δ 9.03 (s, 1H), 8.46-8.39 (m, 2H), 8.31 (d, J=8.2 Hz, 1H), 8.08 (d, J=9.1 Hz, 1H), 7.60 (d, J=8.1 Hz, 1H), 5.36-5.25 (m, 1H), 4.64 (s, 2H), 3.64-3.52 (m, 5H), 3.22-3.17 (m, 1H), 2.80-2.59 (m, 3H), 2.28-2.22 (m, 1H), 2.17-2.12 (m, 1H), 1.70-1.61 (m, 7H), 1.55-1.42 (m, 2H), 1.40-1.29 (m, 1H), 1.24-1.14 (m, 2H).

### Example 40

¹H NMR (400 MHz, DMSO-d₆) δ 9.03 (d, J=2.2 Hz, 1H), 8.45-8.38 (m, 2H), 8.30 (d, J=8.1 Hz, 1H), 8.08 (d, J=9.0 Hz, 1H), 7.62 (d, J=8.2 Hz, 1H), 5.35-5.23 (m, 1H), 4.66 (s, 2H), 3.66 (t, J=5.9 Hz, 2H), 3.59 (s, 3H), 2.53 (t, J=5.8 Hz, 2H), 2.38-2.20 (m, 4H), 1.85-1.76 (m, 2H), 1.74-1.58 (m, 10H), 1.44-1.32 (m, 4H).

### Example 41

¹H NMR (400 MHz, DMSO-d₆) δ 9.02 (s, 1H), 8.46-8.37 (m, 2H), 8.30 (d, J=8.1 Hz, 1H), 8.07 (d, J=9.1 Hz, 1H), 7.60 (d, J=8.1 Hz, 1H), 5.36-5.23 (m, 1H), 4.64 (s, 2H), 3.65 (t, J=5.7 Hz, 2H), 3.58 (s, 3H), 3.22 (s, 3H), 3.20-3.12 (m, 1H), 3.00-2.93 (m, 1H), 2.71-2.64 (m, 1H), 2.58 (t, J=5.7 Hz, 2H), 2.03-1.94 (m, 1H), 1.92-1.84 (m, 2H), 1.65 (d, J=6.7 Hz, 6H), 1.62-1.57 (m, 1H), 1.45-1.31 (m, 1H), 1.11-1.00 (m, 1H).

### Example 42

¹H NMR (400 MHz, DMSO-d₆) δ 9.06 (s, 1H), 8.50-8.40 (m, 2H), 8.33 (d, J=8.0 Hz, 1H), 8.11 (d, J=8.8 Hz, 1H), 7.62 (d, J=8.1 Hz, 1H), 5.38-5.27 (m, 1H), 4.66 (s, 2H), 3.65 (t, J=5.8 Hz, 2H), 3.61 (s, 3H), 2.78-2.56 (m, 6H), 2.34-2.26 (m, 1H), 2.08 (s, 6H), 1.88-1.76 (m, 1H), 1.67 (d, J=6.6 Hz, 6H), 1.61-1.52 (m, 1H).

### Example 43

¹H NMR (400 MHz, DMSO-d₆) δ 9.04 (d, J=2.1 Hz, 1H), 8.48-8.40 (m, 2H), 8.32 (dd, J=8.1, 2.4 Hz, 1H), 8.09 (dd, J=9.1, 1.6 Hz, 1H), 7.62 (d, J=8.1 Hz, 1H), 5.37-5.25 (m, 1H), 4.66 (s, 2H), 3.90-3.81 (m, 1H), 3.66 (t, J=5.9 Hz, 2H), 3.60 (s, 3H), 3.17 (s, 3H), 2.79-2.53 (m, 5H), 2.49-2.41 (m, 1H), 2.01-1.91 (m, 1H), 1.66 (d, J=7.1 Hz, 6H), 1.64-1.59 (m, 1H).

### Example 44

¹H NMR (400 MHz, DMSO-d₆) δ 9.00 (s, 1H), 8.38 (d, J=10.9 Hz, 2H), 8.27 (s, 1H), 8.05 (d, J=7.7 Hz, 1H), 7.59 (s, 1H), 5.33-5.21 (m, 1H), 4.62 (s, 2H), 3.70-3.48 (m, 5H), 3.22-3.04 (m, 4H), 2.79-2.62 (m, 2H), 2.58-2.50 (m, 2H), 2.18-2.00 (m, 2H), 1.86-1.72 (m, 2H), 1.64 (d, J=3.9 Hz, 6H), 1.43-1.30 (m, 2H).

### Example 45

¹H NMR (400 MHz, DMSO-d₆) δ 9.04 (s, 1H), 8.47-8.40 (m, 2H), 8.32 (d, J=7.7 Hz, 1H), 8.09 (d, J=9.0 Hz, 1H), 7.61 (d, J=8.1 Hz, 1H), 5.35-5.26 (m, 1H), 4.65 (s, 2H), 3.65-3.56 (m, 4H), 3.46-3.39 (m, 1H), 2.77 (t, J=6.2 Hz, 2H), 2.70-2.61 (m, 2H), 1.84-1.73 (m, 2H), 1.66 (d, J=6.6 Hz, 6H), 1.30-1.20 (m, 2H), 1.03 (d, J=6.0 Hz, 6H).

### Example 46

¹H NMR (400 MHz, DMSO-d₆) δ 9.03 (s, 1H), 8.47-8.39 (m, 2H), 8.33-8.28 (m, 1H), 8.08 (d, J=9.1 Hz, 1H), 7.60 (d, J=8.1 Hz, 1H), 5.36-5.25 (m, 1H), 4.64 (s, 2H), 3.64 (t, J=5.8 Hz, 2H), 3.59 (s, 3H), 2.75-2.54 (m, 5H), 2.46-2.39 (m, 1H), 2.33-2.26 (m, 1H), 2.07 (s, 6H), 1.84-1.75 (m, 1H), 1.66 (d, J=6.7 Hz, 6H), 1.60-1.50 (m, 1H).

### Example 47

¹H NMR (400 MHz, DMSO-d₆) δ 9.05 (d, J=2.0 Hz, 1H), 8.48-8.40 (m, 2H), 8.32 (dd, J=8.1, 2.3 Hz, 1H), 8.10 (dd, J=9.1, 1.4 Hz, 1H), 7.62 (d, J=8.1 Hz, 1H), 5.37-5.26 (m, 1H), 4.66 (s, 2H), 4.47-4.36 (m, 1H), 3.67 (t, J=5.7 Hz, 2H), 3.60 (s, 3H), 2.77-2.69 (m, 2H), 2.59 (t, J=5.7 Hz, 2H), 2.32 (t, J=9.4 Hz, 2H), 1.96-1.87 (m, 2H), 1.76-1.63 (m, 8H).

### Example 48

¹H NMR (400 MHz, DMSO-d₆) δ 9.03 (s, 1H), 8.46-8.38 (m, 2H), 8.30 (d, J=8.1 Hz, 1H), 8.08 (d, J=9.0 Hz, 1H), 7.60 (d, J=8.1 Hz, 1H), 5.35-5.23 (m, 1H), 4.64 (s, 2H), 3.70-3.52 (m, 5H), 3.08-2.99 (m, 2H), 2.88-2.79 (m, 2H), 2.68 (t, J=5.8 Hz, 2H), 2.35-2.22 (m, 2H), 1.66 (d, J=6.7 Hz, 6H).

### Example 49

¹H NMR (400 MHz, CDCl₃) δ 8.93 (s, 1H), 8.56 (d, J=9.0 Hz, 1H), 8.47 (s, 1H), 8.04 (d, J=7.7 Hz, 1H), 7.87 (d, J=8.9 Hz, 1H), 7.59 (d, J=7.9 Hz, 1H), 5.72-5.45 (m, 2H), 5.30-5.17 (m, 1H), 4.75 (s, 2H), 4.08-3.95 (m, 2H), 3.78 (s, 3H), 3.22-3.12 (m, 2H), 3.12-2.91 (m, 2H), 1.96-1.86 (m, 6H), 1.85-1.76 (m, 10H).

### Example 50

¹H NMR (400 MHz, DMSO-d₆) δ 9.06 (d, J=1.9 Hz, 1H), 8.50-8.41 (m, 2H), 8.36-8.30 (m, 1H), 8.11 (dd, J=9.1, 1.7 Hz, 1H), 7.63 (d, J=8.1 Hz, 1H), 6.68 (t, J=76.0 Hz, 1H), 5.36-5.27 (m, 1H), 4.73-4.62 (m, 3H), 3.67 (t, J=5.8 Hz, 2H), 3.61 (s, 3H), 2.80-2.65 (m, 5H), 2.48-2.40 (m, 1H), 2.22-2.09 (m, 1H), 1.81-1.72 (m, 1H), 1.67 (d, J=6.8 Hz, 6H).

### Example 51

¹H NMR (400 MHz, DMSO-d₆) δ 9.06 (d, J=2.0 Hz, 1H), 8.49-8.42 (m, 2H), 8.36-8.31 (m, 1H), 8.11 (dd, J=9.1, 1.7 Hz, 1H), 7.63 (d, J=8.1 Hz, 1H), 6.67 (t, J=76.0 Hz, 1H), 5.38-5.26 (m, 1H), 4.72-4.63 (m, 3H), 3.67 (t, J=5.8 Hz, 2H), 3.61 (s, 3H), 2.78-2.66 (m, 5H), 2.47-2.40 (m, 1H), 2.20-2.10 (m, 1H), 1.81-1.73 (m, 1H), 1.67 (d, J=6.8 Hz, 6H).

### Example 52

¹H NMR (400 MHz, CDCl₃) δ 8.94 (s, 1H), 8.56 (d, J=8.9 Hz, 1H), 8.44 (s, 1H), 8.07 (d, J=7.3 Hz, 1H), 7.92 (d, J=8.9 Hz, 1H), 7.65 (d, J=8.1 Hz, 1H), 5.14-5.01 (m, 1H), 4.85-4.70 (m, 4H), 4.30-4.18 (m, 2H), 3.83-3.69 (m, 5H), 3.61 (t, J=11.8 Hz, 2H), 3.01-2.92 (m, 1H), 2.90-2.73 (m, 6H), 2.68-2.56 (m, 1H), 2.32-2.21 (m, 1H), 2.10-1.99 (m, 2H).

### Example 53

### (R)-8-(6-(1-(2-(5-azaspiro[2.4]heptan-5-yl)ethoxy)ethyl)pyridin-3-yl)-1-isopropyl-3-methyl-1H-imidazo[4,5-c]cinnolin-2(3H)-one

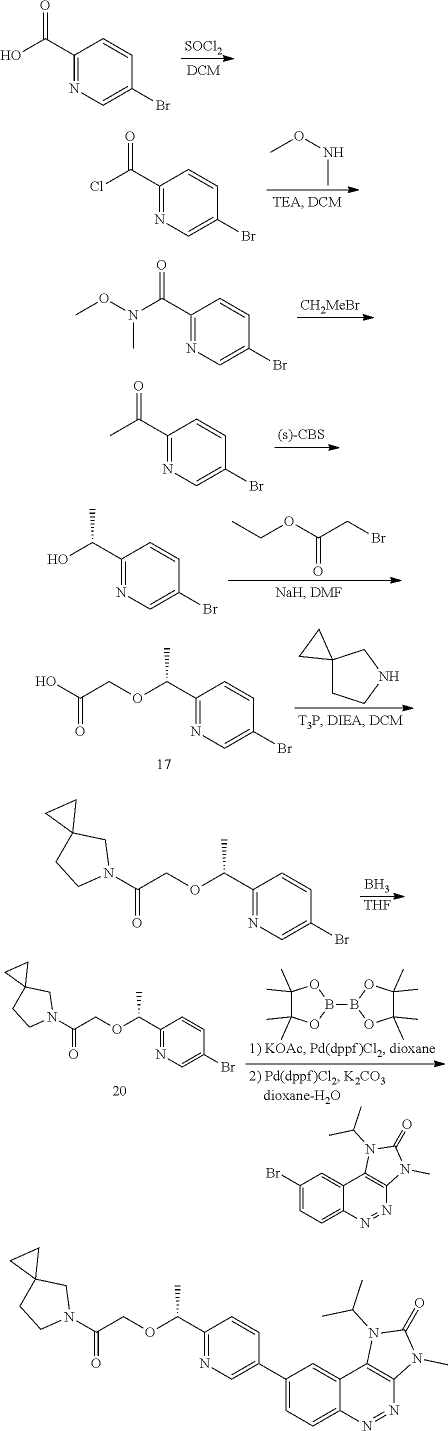

### Step 1: 5-bromopicolinoyl Chloride

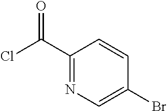

To a solution of 5-bromopicolinic acid (80.0 g, 398 mmol) in DCM (600 mL) was added SOCl₂ (144 mL, 1.99 mol) dropwise at room temperature and the reaction mixture was stirred at room temperature for 3 h. The mixture was evaporated under reduced pressure to give crude product (82.0 g) as white solid, which was used for next step without further purification.

### Step 2: 5-bromo-N-methoxy-N-methylpicolinamide

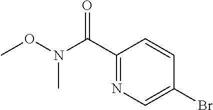

To a solution of N,O-dimethylhydroxylamine hydrochloride (46.6 g, 478 mmol) in DCM (800 mL) was added TEA (283 mL, 1.91 mol), followed by 5-bromopicolinoyl chloride (82.0 g) in portions at 0° C. After stirred at room temperature for 3 h, the mixture was poured into ice sat. NaHCO₃ solution (800 mL) and extracted with DCM (400 mL×2). The combined organic layers were washed with 1 M HCl solution (800 mL×2) and brine (800 mL), dried over anhydrous Na₂SO₄ and filtration, the filtrate was concentrated under reduced pressure to give crude product (67.0 g) as brown oil, which was used for next step without further purification. LC-MS (ESI) m z: 245 [M+H]⁺.

### Step 3: 5-bromo-N-methoxy-N-methylpicolinamide

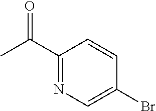

To a solution of 5-bromo-N-methoxy-N-methylpicolinamide (67.0 g, 275 mmol) in dry THE (550 mL) was added MeMgBr (3 M in THF) (137 mL, 413 mmol) dropwise at 0° C. and the reaction mixture was stirred at room temperature for 16 h. The mixture was poured into ice sat. NH₄Cl solution (800 mL) and extracted with EtOAc (400 mL×2). The combined organic layers were washed with brine (800 mL), dried over anhydrous Na₂SO₄ and concentrated under reduced pressure to give crude product, that was triturated with MeOH (200 mL) for 1 h at room temperature and filtered. The filtered cake was dried under vacuum for 3 h to give the desired product (41.5 g, 52.4% yield for three steps) as an off-white solid. LC-MS (ESI) m z: 200 [M+H]⁺.

### Step 4: (R)-1-(5-bromopyridin-2-yl)ethanol

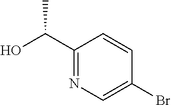

To a solution of (S)-2-Methyl-CBS-oxazaborolidine (42.8 g, 155 mmol) in dry THE (400 mL) was added 1 M BH₃-THF solution (309 mL, 309 mmol) dropwise at 0° C. for about 1 h and the reaction mixture was stirred at 0° C. for 1 h. Then to the mixture was added a solution of 5-bromo-N-methoxy-N-methylpicolinamide (41.5 g, 206 mmol) in dry THF (400 mL) dropwise at −40° C. for about 1.5 h and the resulting mixture was stirred at −40° C. for 1 h. The mixture was poured into ice 1 M HCl solution (800 mL) and stirred at room temperature for 1 h. The solvent was evaporated under reduced pressure, solid precipitate out and filtration. The residue was adjusted with 1 M NaOH solution to pH˜8 and extracted with MTBE (400 mL×2). The organic layer was washed with brine (500 mL), dried over anhydrous Na₂SO₄ and filtration, the filtrate was evaporated under reduced pressure to give the desired product (32.0 g, 99.60% ee, 77.3% yield) as light yellow oil. LC-MS (ESI) m z: 202 [M+H]⁺.

### Step 5: (R)-2-(1-(5-bromopyridin-2-yl)ethoxy)acetic acid

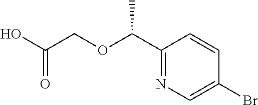

To a solution of (R)-1-(5-bromopyridin-2-yl)ethanol (32.0 g, 159 mmol) in dry DMF (500 mL) was added NaH slowly at 0° C. and the reaction mixture was stirred at 0° C. for 30 min. Then to the mixture was added methyl 2-bromoacetate (21 mL, 207 mmol) dropwise, the resulting reaction mixture was warmed to room temperature slowly and stirred at room temperature for 3 h. The mixture was poured into ice 1 M NaOH solution (420 mL) and stirred at room temperature for 1 h. The mixture was extracted with EtOAc (200 mL×2) and the water layer was adjusted with 1 M HCl solution to pH˜4. It was extracted with EtOAc (200 mL×3), the organic layers were washed with 5% LiCl solution (200 mL) and brine (200 mL), dried over anhydrous Na₂SO₄ and filtration, the filtrate concentrated under reduced pressure to give crude product, which was triturated with MTBE (100 mL) at room temperature for 20 min and filtered. The filtered cake was dried under vacuum for 3 h to give the desired product (26 g, 63.1% yield) as a white solid. ¹H NMR (400 MHz, DMSO-d₆) δ 12.69 (s, 1H), 8.71-8.54 (m, 1H), 8.08 (dd, J=8.4, 2.4 Hz, 1H), 7.47 (d, J=8.4 Hz, 1H), 4.60 (q, J=6.5 Hz, 1H), 3.99 (q, J=37.5, 16.6 Hz, 2H), 1.40 (d, J=6.5 Hz, 3H). LC-MS (ESI) m z: 611.0 [M+H]⁺.

### Step 6: (R)-2-(1-(5-bromopyridin-2-yl)ethoxy)-1-(5-azaspiro[2.4]heptan-5-yl)ethanone

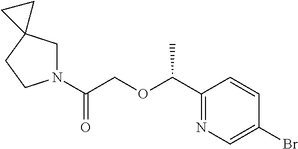 [SMILES: C[C@@H](OCC(=O)N1CCC2(CC2)C1)c1ccc(Br)cn1]

A mixture of (R)-2-(1-(5-bromopyridin-2-yl)ethoxy)acetic acid (19.4 g, 74.9 mmol) and 5-Azaspiro[2.4]heptane hydrochloride (10.0 g, 74.9 mmol) in DCM (250 mL) was added 50% T₃P in EtOAc (95.4 g, 150 mmol), followed by DIEA (49.4 mL, 300 mmol) at 0° C. After stirred at room temperature for 16 h, the mixture was poured into ice sat. NaHCO₃ solution (500 mL) and extracted with DCM (150 mL×2). The organic layer was washed with sat. NH₄Cl solution (200 mL) and brine (200 mL), dried over anhydrous Na₂SO₄ and filtered, the filtrate was concentrated under reduced pressure to give crude product (28.0 g) as brown oil, which was used for next step without further purification. LC-MS (ESI) m z: 339 [M+H]⁺.

### Step 7: (R)-5-(2-(1-(5-bromopyridin-2-yl)ethoxy)ethyl)-5-azaspiro[2.4]heptane

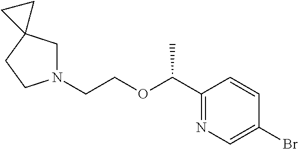 [SMILES: C[C@@H](OCCN1CCC2(CC2)C1)c1ccc(Br)cn1]

To a solution of (R)-2-(1-(5-bromopyridin-2-yl)ethoxy)-1-(5-azaspiro[2.4]heptan-5-yl)ethanone (28.0 g, 82.8 mmol) in dry THE was added 1 M BH₃·THF solution (414 mL, 414 mmol) dropwise at 0° C. and the reaction mixture was stirred at room temperature for 2 h. The mixture was quenched with MeOH (500 mL) and evaporated under reduced pressure. The residue was dissolved in EtOH (300 mL) and stirred at 90° C. for 3 h. The mixture was evaporated under reduced pressure and the residue was purified by chromatography on silica gel (DCM:MeOH=100:1 to 10:1) to give the desired product (12.0 g, 49.0% yield for two steps) as light brown oil. LC-MS (ESI) m z: 325 [M+H]⁺.

### Step 8: (R)-8-(6-(1-(2-(5-azaspiro[2.4]heptan-5-yl)ethoxy)ethyl)pyridin-3-yl)-1-isopropyl-3-methyl-1H-imidazo[4,5-c]cinnolin-2(3H)-one

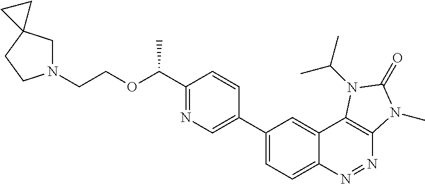 [SMILES: CC(C)n1c(=O)n(C)c2nnc3ccc(-c4ccc([C@@H](C)OCCN5CCC6(CC6)C5)nc4)cc3c21]

The crude product was prepared in a similar fashion to Example 1, which was purified by chromatography on silica gel (DCM:MeOH=100:1 to 10:1) to give the desired product (3.50 g, 23.3% yield for two steps) as an off-white solid. ¹H NMR (400 MHz, CDCl₃) δ 8.92 (s, 1H), 8.55 (d, J=9.0 Hz, 1H), 8.28 (s, 1H), 8.05 (d, J=8.0 Hz, 1H), 7.87 (d, J=9.1 Hz, 1H), 7.61 (d, J=8.2 Hz, 1H), 5.30-5.13 (m, 1H), 4.79-4.64 (m, 1H), 4.02-3.87 (m, 2H), 3.77 (s, 3H), 3.57-3.12 (m, 6H), 2.15-1.93 (m, 2H), 1.79 (d, J=6.9 Hz, 6H), 1.57 (d, J=6.4 Hz, 3H), 0.82-0.62 (m, 4H). LC-MS (ESI) m z: 487 [M+H]⁺.

The following compounds were prepared according to the above described methods using different starting materials.

| | | | MS |
|---|---|---|---|
| Ex# | Structure | Name | m/z |
| 54 | 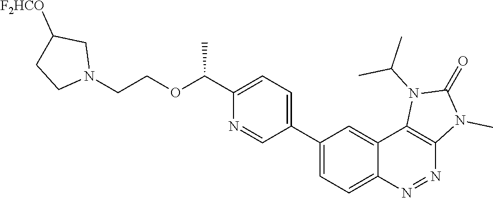 [SMILES: CC(C)n1c(=O)n(C)c2nnc3ccc(-c4ccc([C@@H](C)OCCN5CCC(OC(F)F)C5)nc4)cc3c21] | 8-(6-((1R)-1-(2-(3- (difluoromethoxy)pyrrolidin- 1-yl)ethoxy)ethyl)pyridin-3- yl)-1-isopropyl-3-methyl-1H- imidazo[4,5-c]cinnolin- 2(3H)-one | 526 [M + H]⁺ |
| 55 | 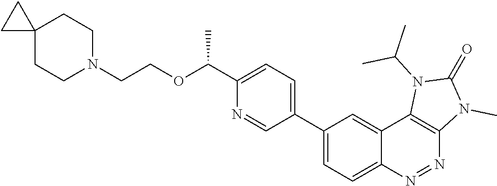 [SMILES: CC(C)n1c(=O)n(C)c2nnc3ccc(-c4ccc([C@@H](C)OCCN5CCC6(CC5)CC6)nc4)cc3c21] | (R)-8-(6-(1-(2-(6- azaspiro[2.5]octan-6- yl)ethoxy)ethyl)pyridin-3-yl)- 1-isopropyl-3-methyl-1H- imidazo[4,5-c]cinnolin- 2(3H)-one | 501 [M + H]⁺ |
| 56 | 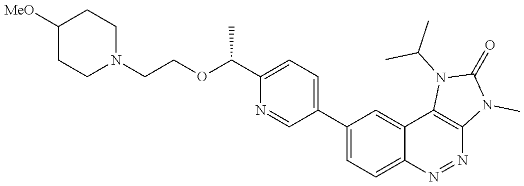 [SMILES: COC1CCN(CCO[C@H](C)c2ccc(-c3ccc4nnc5c(c4c3)n(C(C)C)c(=O)n5C)cn2)CC1] | (R)-1-isopropyl-8-(6-(1-(2- (4-methoxypiperidin-1- yl)ethoxy)ethyl)pyridin-3-yl)- 3-methyl-1H-imidazo[4,5- c]cinnolin-2(3H)-one | 505 [M + H]⁺ |
| 57 | 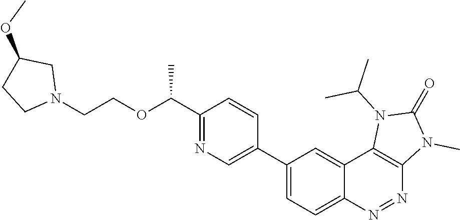 [SMILES: CO[C@@H]1CCN(CCO[C@H](C)c2ccc(-c3ccc4nnc5c(c4c3)n(C(C)C)c(=O)n5C)cn2)C1] | 1-isopropyl-8-(6-((R)-1-(2- ((R)-3-methoxypyrrolidin-1- yl)ethoxy)ethyl)pyridin-3-yl)- 3-methyl-1H-imidazo[4,5- c]cinnolin-2(3H)-one | 491 [M + H]⁺ |
| 58 | 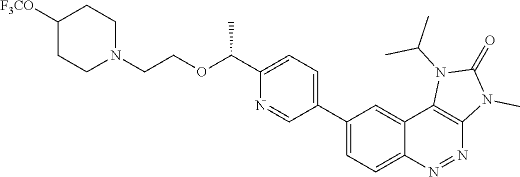 [SMILES: CC(C)n1c(=O)n(C)c2nnc3ccc(-c4ccc([C@@H](C)OCCN5CCC(OC(F)(F)F)CC5)nc4)cc3c21] | (R)-1-isopropyl-3-methyl-8- (6-(1-(2-(4- (trifluoromethoxy)piperidin- 1-yl)ethoxy)ethyl)pyridin-3- yl)-1H-imidazo[4,5- c]cinnolin-2(3H)-one | 559 [M + H]⁺ |
| 59 | 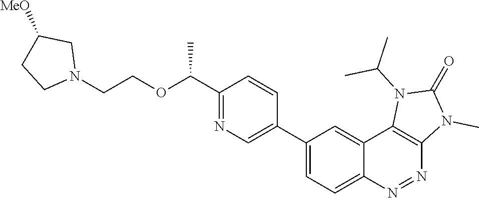 [SMILES: CO[C@H]1CCN(CCO[C@H](C)c2ccc(-c3ccc4nnc5c(c4c3)n(C(C)C)c(=O)n5C)cn2)C1] | 1-isopropyl-8-(6-((R)-1-(2- ((S)-3-methoxypyrrolidin-1- yl)ethoxy)ethyl)pyridin-3-yl)- 3-methyl-1H-imidazo[4,5- c]cinnolin-2(3H)-one | 491 [M + H]⁺ |
| 60 | 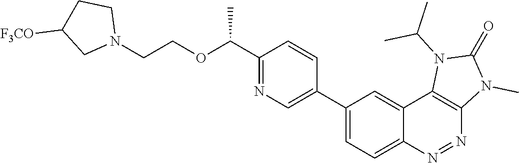 [SMILES: CC(C)n1c(=O)n(C)c2nnc3ccc(-c4ccc([C@@H](C)OCCN5CCC(OC(F)(F)F)C5)nc4)cc3c21] | 8-(6-((2-(3,3- dimethylpyrrolidin-1- yl)ethoxy)methyl)pyridin-3- yl)-7-fluoro-1-isopropyl-3- methyl-1H-imidazo[4,5- c]cinnolin-2(3H)-one | 545 [M + H]⁺ |
| 61 | 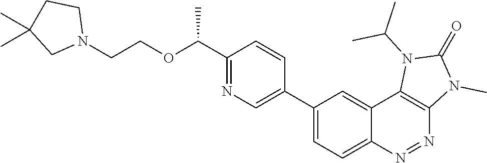 | (R)-8-(6-(1-(2-(3,3- dimethylpyrrolidin-1- yl)ethoxy)ethyl)pyridin-3-yl)- 1-isopropyl-3-methyl-1H- imidazo[4,5-c]cinnolin- 2(3H)-one | 489 [M + H]⁺ |
| 62 | 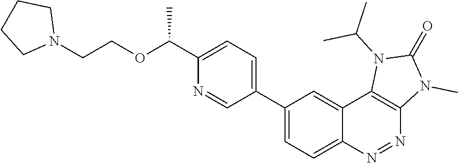 [SMILES: CC(C)n1c(=O)n(C)c2nnc3ccc(-c4ccc([C@@H](C)OCCN5CCCC5)nc4)cc3c21] | (R)-1-isopropyl-3-methyl-8- (6-(1-(2-(pyrrolidin-1- yl)ethoxy)ethyl)pyridin-3-yl)- 1,3-dihydro-2H-imidazo[4,5- c]cinnolin-2-one | 461 [M + H]⁺ |
| 63 | 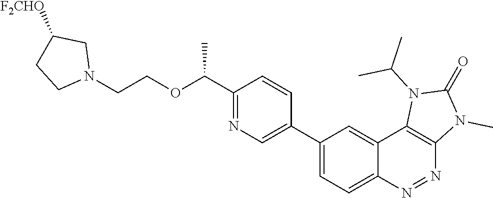 | 8-(6-((R)-1-(2-((S)-3- (difluoromethoxy)pyrrolidin- 1-yl)ethoxy)ethyl)pyridin-3- yl)-1-isopropyl-3-methyl-1H- imidazo[4,5-c]cinnolin- 2(3H)-one | 527 [M + H]⁺ |
| 64 | 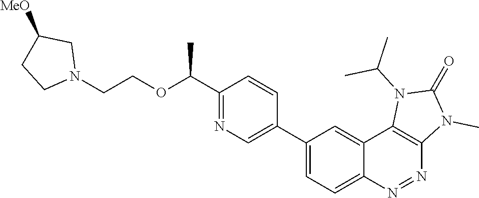 [SMILES: CO[C@@H]1CCN(CCO[C@@H](C)c2ccc(-c3ccc4nnc5c(c4c3)n(C(C)C)c(=O)n5C)cn2)C1] | 1-isopropyl-8-(6-((S)-1-(2- ((R)-3-methoxypyrrolidin-1- yl)ethoxy)ethyl)pyridin-3-yl)- 3-methyl-1H-imidazo[4,5- c]cinnolin-2(3H)-one | 491 [M + H]⁺ |
| 65 | 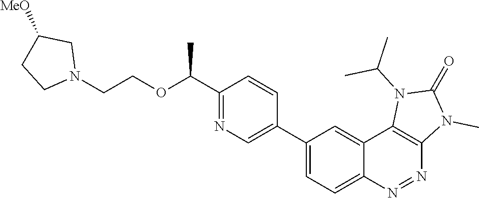 [SMILES: CO[C@H]1CCN(CCO[C@@H](C)c2ccc(-c3ccc4nnc5c(c4c3)n(C(C)C)c(=O)n5C)cn2)C1] | 1-isopropyl-8-(6-((S)-1-(2- ((S)-3-methoxypyrrolidin-1- yl)ethoxy)ethyl)pyridin-3-yl)- 3-methyl-1H-imidazo[4,5- c]cinnolin-2(3H)-one | 491 [M + H]⁺ |
| 66 | 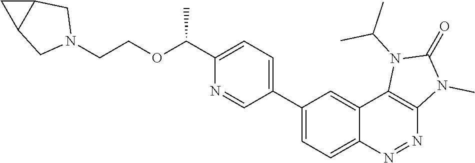 [SMILES: CC(C)n1c(=O)n(C)c2nnc3ccc(-c4ccc([C@@H](C)OCCN5CC6CC6C5)nc4)cc3c21] | 8-(6-((1R)-1-(2-(3- azabicyclo[3.1.0]hexan-3- yl)ethoxy)ethyl)pyridin-3- yl)-1-isopropyl-3-methyl-1H- imidazo[4,5-c]cinnolin- 2(3H)-one | 473 [M + H]⁺ |
| 67 | 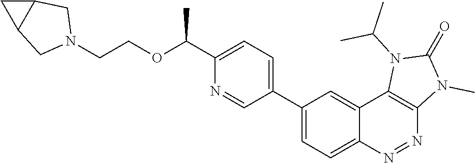 [SMILES: CC(C)n1c(=O)n(C)c2nnc3ccc(-c4ccc([C@H](C)OCCN5CC6CC6C5)nc4)cc3c21] | 8-(6-((1S)-1-(2-(3- azabicyclo[3.1.0]hexan-3- yl)ethoxy)ethyl)pyridin-3- yl)-1-isopropyl-3-methyl-1H- imidazo[4,5-c]cinnolin- 2(3H)-one | 473 [M + H]⁺ |
| 68 | 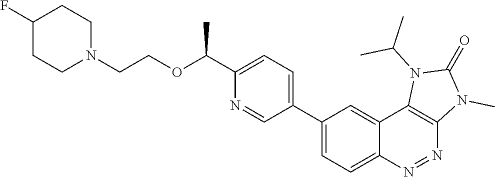 [SMILES: CC(C)n1c(=O)n(C)c2nnc3ccc(-c4ccc([C@H](C)OCCN5CCC(F)CC5)nc4)cc3c21] | (S)-8-(6-(1-(2-(4- fluoropiperidin-1- yl)ethoxy)ethyl)pyridin-3-yl)- 1-isopropyl-3-methyl-1H- imidazo[4,5-c]cinnolin- 2(3H)-one | 493 [M + H]⁺ |
| 69 | 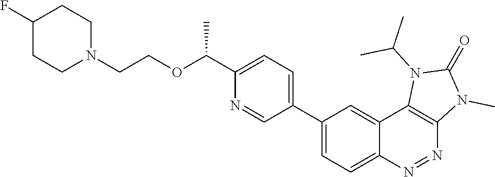 [SMILES: CC(C)n1c(=O)n(C)c2nnc3ccc(-c4ccc([C@@H](C)OCCN5CCC(F)CC5)nc4)cc3c21] | (R)-8-(6-(1-(2-(4- fluoropiperidin-1- yl)ethoxy)ethyl)pyridin-3-yl)- 1-isopropyl-3-methyl-1H- imidazo[4,5-c]cinnolin- 2(3H)-one | 493 [M + H]⁺ |
| 70 | 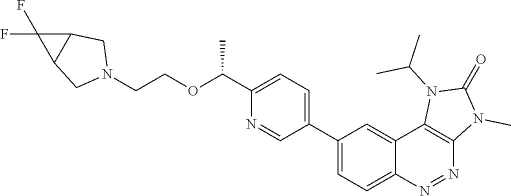 [SMILES: CC(C)n1c(=O)n(C)c2nnc3ccc(-c4ccc([C@@H](C)OCCN5CC6C(C5)C6(F)F)nc4)cc3c21] | 8-(6-((1S)-1-(2-(6,6-difluoro- 3-azabicyclo[3.1.0]hexan-3- yl)ethoxy)ethyl)pyridin-3-yl)- 1-isopropyl-3-methyl-1H- imidazo[4,5-c]cinnolin- 2(3H)-one | 509 [M + H]⁺ |
| 71 | 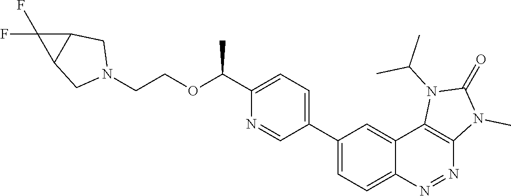 [SMILES: CC(C)n1c(=O)n(C)c2nnc3ccc(-c4ccc([C@H](C)OCCN5CC6C(C5)C6(F)F)nc4)cc3c21] | 8-(6-((1R)-1-(2-(6,6-difluoro- 3-azabicyclo[3.1.0]hexan-3- yl)ethoxy)ethyl)pyridin-3-yl)- 1-isopropyl-3-methyl-1H- imidazo[4,5-c]cinnolin- 2(3H)-one | 509 [M + H]⁺ |
| 72 | 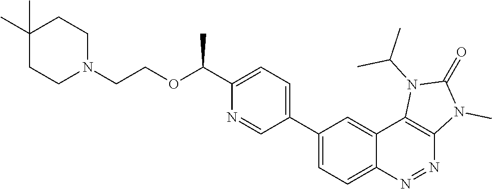 | (S)-8-(6-(1-(2-(4,4- dimethylpiperidin-1- yl)ethoxy)ethyl)pyridin-3-yl)- 1-isopropyl-3-methyl-1H- imidazo[4,5-c]cinnolin- 2(3H)-one | 503 [M + H]⁺ |
| 73 | 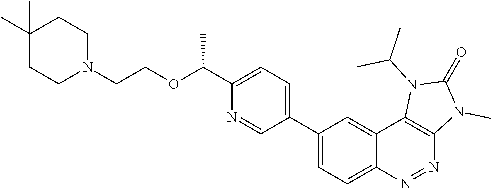 | (R)-8-(6-(1-(2-(4,4- dimethylpiperidin-1- yl)ethoxy)ethyl)pyridin-3-yl)- 1-isopropyl-3-methyl-1H- imidazo[4,5-c]cinnolin- 2(3H)-one | 503 [M + H]⁺ |
| 74 | 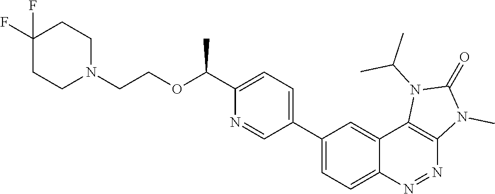 [SMILES: CC(C)n1c(=O)n(C)c2nnc3ccc(-c4ccc([C@H](C)OCCN5CCC(F)(F)CC5)nc4)cc3c21] | (S)-8-(6-(1-(2-(4,4- difluoropiperidin-1- yl)ethoxy)ethyl)pyridin-3-yl)- 1-isopropyl-3-methyl-1H- imidazo[4,5-c]cinnolin- 2(3H)-one | 511 [M + H]⁺ |
| 75 | 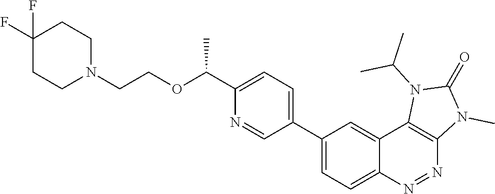 [SMILES: CC(C)n1c(=O)n(C)c2nnc3ccc(-c4ccc([C@@H](C)OCCN5CCC(F)(F)CC5)nc4)cc3c21] | (R)-8-(6-(1-(2-(4,4- difluoropiperidin-1- yl)ethoxy)ethyl)pyridin-3-yl)- 1-isopropyl-3-methyl-1H- imidazo[4,5-c]cinnolin- 2(3H)-one | 511 [M + H]⁺ |

### Example 54

¹H NMR (400 MHz, CDCl₃) δ 8.92 (d, J=1.9 Hz, 1H), 8.56 (d, J=9.0 Hz, 1H), 8.28 (s, 2H), 8.05 (dd, J=8.1, 2.3 Hz, 1H), 7.88 (dd, J=9.0, 1.7 Hz, 1H), 7.63 (d, J=8.1 Hz, 1H), 6.24 (t, J=73.6 Hz, 1H), 5.27-5.17 (m, 1H), 4.92-4.85 (m, 1H), 4.68 (q, J=6.5 Hz, 1H), 3.80-3.69 (m, 5H), 3.48-3.40 (m, 1H), 3.21-2.99 (m, 5H), 2.39-2.28 (m, 1H), 1.79 (d, J=7.0 Hz, 6H), 1.57 (d, J=6.5 Hz, 3H), 0.90-0.79 (m, 1H).

### Example 55

¹H NMR (400 MHz, DMSO-d₆) δ 9.05 (d, J=1.9 Hz, 1H), 8.51-8.42 (m, 2H), 8.33 (dd, J=8.2, 2.4 Hz, 1H), 8.10 (dd, J=9.1, 1.6 Hz, 1H), 7.66 (d, J=8.2 Hz, 1H), 5.40-5.28 (m, 1H), 4.62 (q, J=6.5 Hz, 1H), 3.62-3.57 (m, 4H), 3.54-3.44 (m, 1H), 2.70-2.58 (m, 2H), 2.55-2.52 (m, 4H), 1.67 (d, J=6.7 Hz, 6H), 1.45 (d, J=6.5 Hz, 3H), 1.35 (s, 4H), 0.25 (s, 4H).

### Example 56

¹H NMR (400 MHz, DMSO-d₆) δ 9.05 (d, J=1.9 Hz, 1H), 8.51-8.40 (m, 2H), 8.32 (dd, J=8.2, 2.3 Hz, 1H), 8.10 (dd, J=9.1, 1.4 Hz, 1H), 7.64 (d, J=8.2 Hz, 1H), 5.38-5.25 (m, 1H), 4.60 (q, J=6.5 Hz, 1H), 3.60 (s, 3H), 3.57-3.53 (m, 1H), 3.49-3.44 (m, 1H), 3.21 (s, 3H), 3.18-3.12 (m, 1H), 2.78-2.70 (m, 2H), 2.62-2.53 (m, 2H), 2.24-2.13 (m, 2H), 1.86-1.77 (m, 2H), 1.67 (d, J=6.7 Hz, 6H), 1.48-1.37 (m, 5H).

### Example 57

¹H NMR (400 MHz, DMSO-d₆) δ 9.03 (s, 1H), 8.49-8.38 (m, 2H), 8.30 (d, J=7.8 Hz, 1H), 8.08 (d, J=9.0 Hz, 1H), 7.62 (d, J=8.4 Hz, 1H), 5.36-5.26 (m, 1H), 4.63-4.54 (m, 1H), 3.88-3.81 (m, 1H), 3.61-3.55 (m, 5H), 3.15 (s, 3H), 2.77-2.52 (m, 6H), 2.00-1.90 (m, 1H), 1.72-1.55 (m, 7H), 1.43 (d, J=6.3 Hz, 3H).

### Example 58

¹H NMR (400 MHz, DMSO-d₆) δ 9.03 (s, 1H), 8.48-8.38 (m, 2H), 8.31 (d, J=8.2 Hz, 1H), 8.08 (d, J=9.1 Hz, 1H), 7.63 (d, J=8.2 Hz, 1H), 5.36-5.25 (m, 1H), 4.58 (q, J=6.3 Hz, 1H), 4.45-4.35 (m, 1H), 3.59 (s, 3H), 3.56-3.49 (m, 1H), 3.47-3.39 (m, 1H), 2.73-2.63 (m, 2H), 2.58-2.51 (m, 2H), 2.32-2.21 (m, 2H), 1.95-1.84 (m, 2H), 1.73-1.58 (m, 8H), 1.42 (d, J=6.5 Hz, 3H).

### Example 59

¹H NMR (400 MHz, DMSO-d₆) δ 9.05 (d, J=2.0 Hz, 1H), 8.50-8.41 (m, 2H), 8.33 (dd, J=8.2, 2.3 Hz, 1H), 8.10 (dd, J=9.1, 1.6 Hz, 1H), 7.63 (d, J=8.2 Hz, 1H), 5.39-5.27 (m, 1H), 4.60 (q, J=6.5 Hz, 1H), 3.94-3.81 (m, 1H), 3.60 (s, 3H), 3.58-3.53 (m, 1H), 3.48-3.43 (m, 1H), 3.16 (s, 3H), 2.76-2.52 (m, 6H), 2.01-1.91 (m, 1H), 1.72-1.59 (m, 7H), 1.44 (d, J=6.5 Hz, 3H).

### Example 60

¹H NMR (400 MHz, DMSO-d₆) δ 9.05 (d, J=1.8 Hz, 1H), 8.51-8.40 (m, 2H), 8.35-8.27 (m, 1H), 8.10 (d, J=9.1 Hz, 1H), 7.63 (dd, J=8.1, 2.1 Hz, 1H), 5.37-5.27 (m, 1H), 4.93 (s, 1H), 4.61 (q, J=6.4 Hz, 1H), 3.64-3.53 (m, 4H), 3.50-3.43 (m, 1H), 2.87-2.56 (m, 5H), 2.42-2.32 (m, 1H), 2.28-2.15 (m, 1H), 1.90-1.79 (m, 1H), 1.67 (d, J=6.7 Hz, 6H), 1.44 (d, J=6.5 Hz, 3H).

### Example 61

¹H NMR (400 MHz, DMSO-d₆) δ 9.05 (d, J=1.9 Hz, 1H), 8.50-8.41 (m, 2H), 8.32 (dd, J=8.2, 2.4 Hz, 1H), 8.10 (dd, J=9.1, 1.6 Hz, 1H), 7.64 (d, J=8.2 Hz, 1H), 5.33 (dt, J=13.5, 6.7 Hz, 1H), 4.61 (q, J=6.5 Hz, 1H), 3.60 (s, 3H), 3.57-3.53 (m, 1H), 3.49-3.42 (m, 1H), 2.75-2.60 (m, 4H), 2.37 (s, 2H), 1.67 (d, J=6.7 Hz, 6H), 1.52 (t, J=7.1 Hz, 2H), 1.44 (d, J=6.5 Hz, 3H), 1.04 (d, J=2.4 Hz, 6H).

### Example 62

¹H NMR (400 MHz, CDCl₃) δ 8.88 (s, 1H), 8.53 (d, J=9.0 Hz, 1H), 8.25 (s, 1H), 8.01 (dd, J=8.1, 1.4 Hz, 1H), 7.86 (d, J=9.0 Hz, 1H), 7.65 (d, J=8.1 Hz, 1H), 5.28-5.11 (m, 1H), 4.64 (q, J=6.5 Hz, 1H), 3.76 (s, 3H), 3.67-3.52 (m, 2H), 2.82-2.67 (m, 2H), 2.63-2.50 (m, 4H), 1.87-1.69 (m, 10H), 1.55 (d, J=6.5 Hz, 3H).

### Example 63

¹H NMR (400 MHz, DMSO-d₆) δ 9.04 (d, J=2.0 Hz, 1H), 8.45 (dd, J=11.9, 5.2 Hz, 2H), 8.32 (dd, J=8.2, 2.4 Hz, 1H), 8.10 (dd, J=9.1, 1.6 Hz, 1H), 7.64 (d, J=8.2 Hz, 1H), 6.66 (t, J=76.0 Hz, 1H), 5.36-5.26 (m, 1H), 4.70-4.64 (m, 1H), 4.60 (q, J=6.5 Hz, 1H), 3.60 (s, 3H), 3.58-3.52 (m, 2H), 2.77-2.59 (m, 5H), 2.44-2.36 (m, 1H), 2.19-2.07 (m, 1H), 1.80-1.70 (m, 1H), 1.67 (d, J=6.7 Hz, 6H), 1.44 (d, J=6.5 Hz, 3H).

### Example 64

¹H NMR (400 MHz, CDCl₃) δ 8.92 (d, J=2.0 Hz, 1H), 8.56 (d, J=9.0 Hz, 1H), 8.28 (s, 1H), 8.05 (dd, J=8.1, 2.3 Hz, 1H), 7.88 (dd, J=9.0, 1.7 Hz, 1H), 7.64 (d, J=8.1 Hz, 1H), 5.28-5.18 (m, 1H), 4.69 (q, J=6.4 Hz, 1H), 4.06-3.99 (m, 1H), 3.86-3.68 (m, 5H), 3.49-3.39 (m, 2H), 3.35-3.23 (m, 4H), 3.14-2.96 (m, 4H), 2.20-2.11 (m, 1H), 1.80 (d, J=7.0 Hz, 6H), 1.58 (d, J=6.5 Hz, 3H).

### Example 65

¹H NMR (400 MHz, CDCl₃) δ 8.92 (s, 1H), 8.55 (d, J=9.0 Hz, 1H), 8.29 (s, 1H), 8.07 (d, J=7.7 Hz, 1H), 7.88 (d, J=9.0 Hz, 1H), 7.66 (d, J=8.0 Hz, 1H), 5.29-5.17 (m, 1H), 4.78-4.64 (m, 1H), 4.13-4.03 (m, 1H), 3.92-3.83 (m, 2H), 3.78 (s, 3H), 3.33 (s, 3H), 3.27-3.05 (m, 4H), 2.30-1.99 (m, 4H), 1.80 (d, J=6.8 Hz, 6H), 1.59 (d, J=5.8 Hz, 3H).

### Example 66

¹H NMR (400 MHz, DMSO-d₆) δ 9.02 (s, 1H), 8.52-8.35 (m, 2H), 8.30 (d, J=8.1 Hz, 1H), 8.08 (d, J=9.0 Hz, 1H), 7.60 (d, J=8.2 Hz, 1H), 5.38-5.18 (m, 1H), 4.64-4.44 (m, 1H), 3.59 (s, 3H), 3.51-3.44 (m, 1H), 3.42-3.36 (m, 1H), 2.92 (t, J=8.1 Hz, 2H), 2.65-2.53 (m, 2H), 2.28 (t, J=6.9 Hz, 2H), 1.66 (d, J=6.5 Hz, 6H), 1.41 (d, J=6.4 Hz, 3H), 1.35-1.26 (m, 2H), 0.59-0.52 (m, 1H), 0.32-0.22 (m, 1H).

### Example 67

¹H NMR (400 MHz, DMSO-d₆) δ 9.03 (s, 1H), 8.49-8.36 (m, 2H), 8.30 (d, J=8.1 Hz, 1H), 8.08 (d, J=9.1 Hz, 1H), 7.60 (d, J=8.1 Hz, 1H), 5.35-5.23 (m, 1H), 4.63-4.46 (m, 1H), 3.59 (s, 3H), 3.52-3.45 (m, 1H), 3.43-3.36 (m, 1H), 2.92 (t, J=8.1 Hz, 2H), 2.65-2.53 (m, 2H), 2.28 (t, J=6.8 Hz, 2H), 1.66 (d, J=6.5 Hz, 6H), 1.41 (d, J=6.4 Hz, 3H), 1.35-1.28 (m, 2H), 0.59-0.52 (m, 1H), 0.31-0.22 (m, 1H).

### Example 68

¹H NMR (400 MHz, DMSO-d₆) δ 9.02 (s, 1H), 8.47-8.37 (m, 2H), 8.30 (d, J=7.5 Hz, 1H), 8.07 (d, J=9.1 Hz, 1H), 7.62 (d, J=7.6 Hz, 1H), 5.37-5.24 (m, 1H), 4.74-4.50 (m, 2H), 3.62-3.39 (m, 5H), 2.60-2.50 (m, 4H), 2.35-2.24 (m, 2H), 1.88-1.74 (m, 2H), 1.71-1.57 (m, 8H), 1.42 (d, J=6.1 Hz, 3H).

### Example 69

¹H NMR (400 MHz, DMSO-d₆) δ 9.02 (s, 1H), 8.46-8.38 (m, 2H), 8.30 (d, J=8.3 Hz, 1H), 8.08 (d, J=9.3 Hz, 1H), 7.62 (d, J=8.1 Hz, 1H), 5.37-5.24 (m, 1H), 4.73-4.52 (m, 2H), 3.58 (s, 3H), 3.56-3.49 (m, 1H), 3.48-3.40 (m, 1H), 2.60-2.51 (m, 4H), 2.35-2.26 (m, 2H), 1.89-1.74 (m, 2H), 1.72-1.59 (m, 8H), 1.42 (d, J=6.1 Hz, 3H).

### Example 70

¹H NMR (400 MHz, DMSO-d₆) δ 9.02 (s, 1H), 8.48-8.37 (m, 2H), 8.29 (d, J=7.3 Hz, 1H), 8.08 (d, J=8.7 Hz, 1H), 7.61 (d, J=7.7 Hz, 1H), 5.37-5.21 (m, 1H), 4.65-4.52 (m, 1H), 3.58 (s, 3H), 3.54-3.42 (m, 2H), 3.00 (t, J=10.5 Hz, 2H), 2.85-2.73 (m, 2H), 2.69-2.56 (m, 2H), 2.34-2.23 (m, 2H), 1.66 (d, J=6.6 Hz, 6H), 1.42 (d, J=6.5 Hz, 3H).

### Example 71

¹H NMR (400 MHz, DMSO-d₆) δ 9.03 (s, 1H), 8.49-8.39 (m, 2H), 8.30 (d, J=8.1 Hz, 1H), 8.09 (d, J=9.0 Hz, 1H), 7.62 (d, J=8.1 Hz, 1H), 5.38-5.25 (m, 1H), 4.68-4.53 (m, 1H), 3.59 (s, 3H), 3.53-3.42 (m, 2H), 3.01 (t, J=10.1 Hz, 2H), 2.84-2.75 (m, 2H), 2.69-2.57 (m, 2H), 2.33-2.25 (m, 2H), 1.67 (d, J=6.6 Hz, 6H), 1.43 (d, J=6.4 Hz, 3H).

### Example 72

¹H NMR (400 MHz, DMSO-d₆) δ 9.04 (d, J=2.3 Hz, 1H), 8.55-8.39 (m, 2H), 8.32 (dd, J=8.2, 2.4 Hz, 1H), 8.09 (dd, J=9.1, 1.7 Hz, 1H), 7.64 (d, J=8.2 Hz, 1H), 5.38-5.24 (m, 1H), 4.67-4.49 (m, 1H), 3.60 (s, 3H), 3.56-3.50 (m, 1H), 3.48-3.41 (m, 1H), 2.59-2.52 (m, 2H), 2.41-2.28 (m, 4H), 1.67 (d, J=6.7 Hz, 6H), 1.43 (d, J=6.5 Hz, 3H), 1.29 (t, J=5.6 Hz, 4H), 0.87 (s, 6H).

### Example 73

¹H NMR (400 MHz, DMSO-d₆) δ 9.03 (s, 1H), 8.48-8.38 (m, 2H), 8.31 (dd, J=8.2, 2.2 Hz, 1H), 8.08 (dd, J=9.1, 1.8 Hz, 1H), 7.63 (d, J=8.2 Hz, 1H), 5.40-5.24 (m, 1H), 4.62-4.49 (m, 1H), 3.59 (s, 3H), 3.57-3.51 (m, 1H), 3.47-3.40 (m, 1H), 2.55-2.51 (m, 2H), 2.35 (t, J=5.7 Hz, 4H), 1.67 (d, J=6.7 Hz, 6H), 1.43 (d, J=6.6 Hz, 3H), 1.29 (t, J=5.6 Hz, 4H), 0.87 (s, 6H).

### Example 74

¹H NMR (400 MHz, DMSO-d₆) δ 9.03 (s, 1H), 8.49-8.39 (m, 2H), 8.31 (d, J=8.2 Hz, 1H), 8.08 (d, J=9.1 Hz, 1H), 7.62 (d, J=8.2 Hz, 1H), 5.37-5.25 (m, 1H), 4.62-4.51 (m, 1H), 3.63-3.52 (m, 4H), 3.50-3.42 (m, 1H), 2.68-2.51 (m, 6H), 1.99-1.84 (m, 4H), 1.66 (d, J=6.6 Hz, 6H), 1.43 (d, J=6.5 Hz, 3H).

### Example 75

¹H NMR (400 MHz, DMSO) δ 9.04 (d, J=2.4, 0.8 Hz, 1H), 8.49-8.40 (m, 2H), 8.33 (dd, J=8.2, 2.4 Hz, 1H), 8.09 (dd, J=9.0, 1.7 Hz, 1H), 7.63 (d, J=8.1 Hz, 1H), 5.38-5.27 (m, 1H), 4.64-4.54 (m, 1H), 3.59 (s, 3H), 3.58-3.54 (m, 1H), 3.50-3.43 (m, 1H), 2.67-2.57 (m, 2H), 2.57-2.52 (m, 4H), 2.01-1.85 (m, 4H), 1.67 (d, J=6.7 Hz, 6H), 1.43 (d, J=6.5 Hz, 2H).

### Example 76

### 8-(6-((R)-1-(2-((R)-3-cyclopropoxypyrrolidin-1-yl)ethoxy)ethyl) pyridin-3-yl)-1-isopropyl-3-methyl-1H-imidazo[4,5-c]cinnolin-2(3H)-one

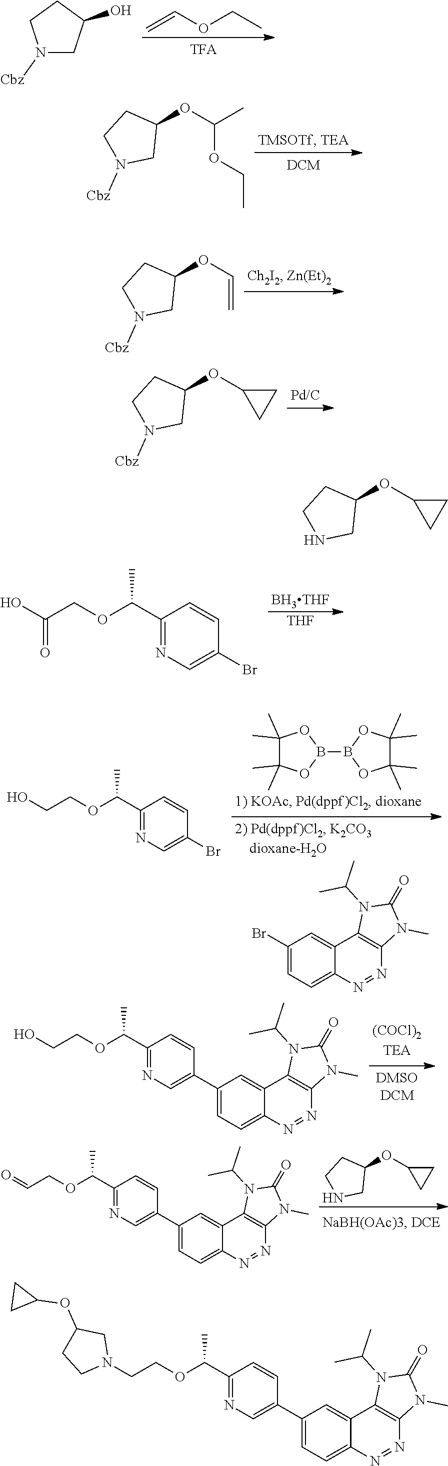

### Step 1: (3R)-benzyl 3-(1-ethoxyethoxy)pyrrolidine-1-carboxylate

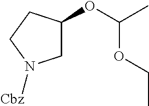 [SMILES: CCOC(C)O[C@@H]1CCN(C(=O)OCc2ccccc2)C1]

To a solution of (R)-benzyl 3-hydroxypyrrolidine-1-carboxylate (3.00 g, 13.6 mmol) in ethoxyethane (15 mL) and DCM (5 mL) was added 2 drops TFA at 0° C. and the reaction mixture was stirred at room temperature for 48 h. The mixture was evaporated under reduced pressure. The residue was purified by chromatography on silica gel (PE:EtOAc=1:0 to 10:1) to give the desired product (2.70 g, 67.8% yield) as light oil.

### Step 2: (R)-benzyl 3-(vinyloxy)pyrrolidine-1-carboxylate

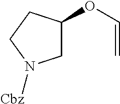 [SMILES: C=CO[C@@H]1CCN(C(=O)OCc2ccccc2)C1]

To a solution of (3R)-benzyl 3-(1-ethoxyethoxy)pyrrolidine-1-carboxylate (2.70 g, 9.22 mmol) in anhydrous DCM (30 mL) was added TEA (0.684 mL, 4.61 mmol) followed by TMSOTF (0.833 mL, 4.61 mmol) dropwise at 0° C. under N₂. Then the reaction mixture was warmed to room temperature and stirred for 2 h. The mixture was once more cooled to 0° C., and another TEA (0.684 mL, 4.61 mmol) and TMSOTF (0.833 mL, 4.61 mmol) was dropwise added. Then the reaction mixture was slowly warmed to room temperature and stirred overnight. The mixture was poured into ice 1 M NaOH solution (50 mL) and extracted with DCM (20 mL×2). The combined organic layers were washed with brine, dried over anhydrous Na₂SO₄ and filtration, the filtrate was concentrated under reduced pressure. The residue was purified by chromatography on silica gel (PE:EtOAc=1:0 to 10:1) to give the desired product (319 mg, 14.0% yield) as light oil. LC-MS (ESI) m z: 248 [M+H]⁺.

### Step 3: (R)-benzyl 3-cyclopropoxypyrrolidine-1-carboxylate

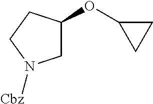 [SMILES: O=C(OCc1ccccc1)N1CC[C@@H](OC2CC2)C1]

1 M diethylzinc solution in hexane (3.23 mL, 3.23 mmol) was added into dry DCM (15 mL), and a solution of diiodomethane (0.260 mL, 3.23 mmol) in dry DCM (3 mL) was added dropwise at 0° C. under N₂. Then the resulting reaction mixture was stirred at 0° C. for 30 min. To the mixture was added dropwise a solution of (R)-benzyl 3-(vinyloxy)pyrrolidine-1-carboxylate (319 mg, 1.29 mmol) at 0° C. The reaction mixture was slowly warmed to ROOM TEMPERATURE and stirred overnight. The mixture was poured into ice sat. NH₄Cl solution (30 mL) and extracted with DCM (20 mL×2). The combined organic layers were washed with sat. NaHCO₃ solution (40 mL), dried over anhydrous Na₂SO₄ and filtration, the filtrate was concentrated under reduced pressure. The residue was purified by chromatography on silica gel (PE:EtOAc=1:0 to 10:1) to give the desired product (182 mg, 54.1% yield) as light oil. LC-MS (ESI) m z: 262 [M+H]⁺.

### Step 4: (R)-3-cyclopropoxypyrrolidine

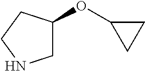

To a solution of (R)-benzyl 3-cyclopropoxypyrrolidine-1-carboxylate (150 mg, 0.575 mmol) in MeOH (3 mL) and 6 M HCl solution (0.5 mL) was added Pd/C (15.0 mg) and the reaction mixture was stirred at room temperature overnight. The mixture was filtered and the filtrate was evaporated under reduced pressure to give the crude desired product (104 mg) as light brown oil. LC-MS (ESI) m z: 128 [M+H]⁺.

### Step 5: (R)-2-(1-(5-bromopyridin-2-yl)ethoxy)ethanol

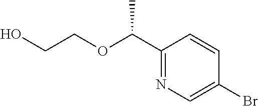

To a solution of (R)-2-(1-(5-bromopyridin-2-yl)ethoxy)acetic acid (9.50 g, 36.7 mmol) in THE (100 mL) was added dropwise 1 M BH₃·THF solution (73.4 mL, 73.4 mmol) at 0° C. under N₂. Then the reaction mixture was stirred at room temperature for 1 h. The mixture was quenched with MeOH (100 mL) at 0° C. and stirred at 80° C. for 1 h. The mixture was evaporated under reduced pressure. The residue was purified by chromatography on silica gel (DCM:MeOH=100:1 to 20:1) to give the desired product (6.20 g, 69.0% yield) as light yellow oil. LC-MS (ESI) m z: 246 [M+H]⁺.

### Step 6: (R)-8-(6-(1-(2-hydroxyethoxy)ethyl)pyridin-3-yl)-1-isopropyl-3-methyl-1H-imidazo[4,5-c]cinnolin-2(3H)-one

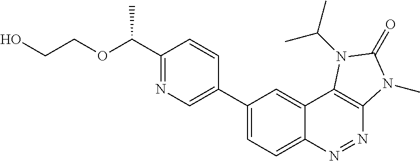 [SMILES: CC(C)n1c(=O)n(C)c2nnc3ccc(-c4ccc([C@@H](C)OCCO)nc4)cc3c21]

The crude product was prepared in a similar fashion to Example 1, which was purified by chromatography on silica gel (DCM:MeOH=100:1 to 10:1) to give the desired product (1.10 g, 66.3% yield) as a brown solid. LC-MS (ESI) m z: 408 [M+H]⁺.

### Step 7: (R)-2-(1-(5-(1-isopropyl-3-methyl-2-oxo-2,3-dihydro-1H-imidazo[4,5-c]cinnolin-8-yl)pyridin-2-yl)ethoxy)acetaldehyde

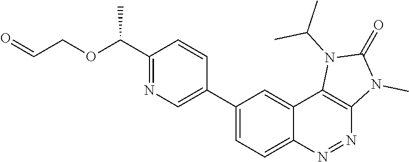 [SMILES: CC(C)n1c(=O)n(C)c2nnc3ccc(-c4ccc([C@@H](C)OCC=O)nc4)cc3c21]

To a solution of oxalyl chloride (0.343 mL, 4.05 mmol) in dry DCM (5 mL) was added dropwise DMSO (0.574 mL, 8.10 mmol) at −78° C. under N₂ and the resulting reaction mixture was stirred at −78° C. for 15 min. Then to the reaction mixture was added dropwise a solution of (R)-8-(6-(1-(2-hydroxyethoxy)ethyl)pyridin-3-yl)-1-isopropyl-3-methyl-1H-imidazo[4,5-c]cinnolin-2(3H)-one (1.10 g, 2.70 mmol) in dry DCM (10 mL) at −78° C. After stirring for 20 min at −78° C., TEA (1.87 mL, 13.5 mmol) was added. The resulting mixture was slowly warmed to ROOM TEMPERATURE. The mixture was quenched with water (30 mL) and extracted with EtOAc (20 mL×2). The combined organic layers were washed with brine (40 mL), dried over anhydrous Na₂SO₄ and concentrated under reduced pressure to give the crude desired product (1.10 g) as a brown solid. LC-MS (ESI) m z: 406 [M+H]⁺.

### Step 8: 8-(6-((R)-1-(2-((R)-3-cyclopropoxypyrrolidin-1-yl)ethoxy)ethyl) pyridin-3-yl)-1-isopropyl-3-methyl-1H-imidazo[4,5-c]cinnolin-2(3H)-one

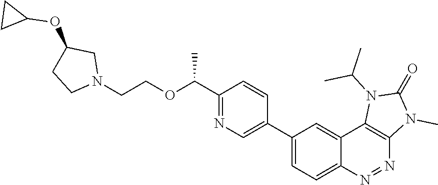 [SMILES: CC(C)n1c(=O)n(C)c2nnc3ccc(-c4ccc([C@@H](C)OCCN5CC[C@@H](OC6CC6)C5)nc4)cc3c21]

To a mixture of (R)-3-cyclopropoxypyrrolidine (50.0 mg, 0.394 mmol) and (R)-2-(1-(5-(1-isopropyl-3-methyl-2-oxo-2,3-dihydro-1H-imidazo[4,5-c]cinnolin-8-yl)pyridin-2-yl)ethoxy) acetaldehyde (159 mg, 0.394 mmol) in DCE (4 mL) was added slowly sodium triacetoxyborohydride (251 mg, 1.18 mmol) at 0° C., the reaction mixture was stirred at room temperature overnight. The mixture was poured into ice sat. NaHCO₃ solution (20 mL) and extracted with DCM (15 mL×2). The organic layers were washed with brine (30 mL), dried over anhydrous Na₂SO₄ and concentrated under reduced pressure. The residue was purified by flash chromatography (DCM:MeOH=9:1) to give the desired product (40.0 mg, 19.7% yield) as a brown solid. ¹H NMR (400 MHz, CDCl₃) δ 8.91 (s, 1H), 8.55 (d, J=9.0 Hz, 1H), 8.28 (s, 1H), 8.05 (dd, J=8.1, 2.1 Hz, 1H), 7.88 (d, J=9.0 Hz, 1H), 7.65 (d, J=8.2 Hz, 1H), 5.28-5.16 (m, 1H), 4.68 (q, J=6.5 Hz, 1H), 4.30-4.20 (m, 1H), 3.83-3.70 (m, 5H), 3.55-3.41 (m, 1H), 3.31-3.23 (m, 1H), 3.17-2.96 (m, 4H), 2.27-2.05 (m, 3H), 1.79 (d, J=6.9 Hz, 6H), 1.57 (d, J=6.5 Hz, 3H), 0.65-0.41 (m, 4H). LC-MS (ESI) m z: 517 [M+H]⁺.

The following compounds were prepared according to the above described methods using different starting materials.

| | | | MS |
|---|---|---|---|
| Ex# | Structure | Name | m/z |
| 77 | 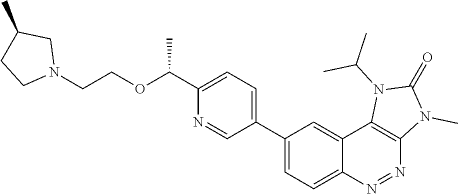 [SMILES: CC(C)n1c(=O)n(C)c2nnc3ccc(-c4ccc([C@@H](C)OCCN5CC[C@@H](C)C5)nc4)cc3c21] | 1-isopropyl-3-methyl-8-(6- ((R)-1-(2-((R)-3- methylpyrrolidin-1- yl)ethoxy)ethyl)pyridin-3-yl)- 1H-imidazo[4,5-c]cinnolin- 2(3H)-one | 475 [M + H]⁺ |
| 78 | 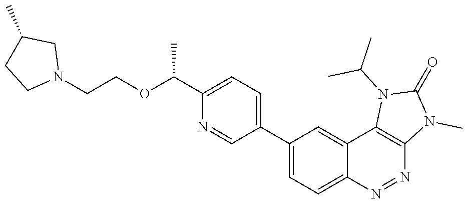 [SMILES: CC(C)n1c(=O)n(C)c2nnc3ccc(-c4ccc([C@@H](C)OCCN5CC[C@H](C)C5)nc4)cc3c21] | 1-isopropyl-3-methyl-8-(6- ((R)-1-(2-((S)-3- methylpyrrolidin-1- yl)ethoxy)ethyl)pyridin-3-yl)- 1H-imidazo[4,5-c]cinnolin- 2(3H)-one | 475 [M + H]⁺ |
| 79 | 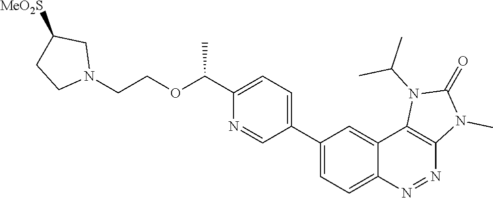 [SMILES: CC(C)n1c(=O)n(C)c2nnc3ccc(-c4ccc([C@@H](C)OCCN5CC[C@@H](S(C)(=O)=O)C5)nc4)cc3c21] | 1-isopropyl-3-methyl-8-(6- ((R)-1-(2-((R)-3- (methylsulfonyl)pyrrolidin-1- yl)ethoxy)ethyl)pyridin-3-yl)- 1H-imidazo[4,5-c]cinnolin- 2(3H)-one | 539 [M + H]⁺ |
| 80 | 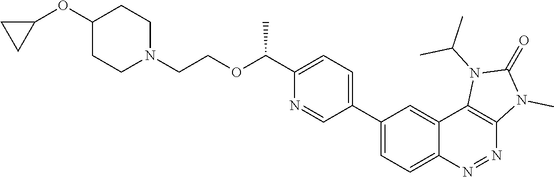 [SMILES: CC(C)n1c(=O)n(C)c2nnc3ccc(-c4ccc([C@@H](C)OCCN5CCC(OC6CC6)CC5)nc4)cc3c21] | 8-(6-((R)-1-(2-((R)-3- cyclopropoxypyrrolidin-1- yl)ethoxy)ethyl)pyridin-3- yl)-3-methyl-1-(tetrahydro- 2H-pyran-4-yl)-1H- imidazo[4,5-c]cinnolin- 2(3H)-one | 531 [M + H]⁺ |
| 81 | 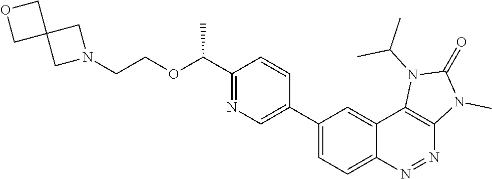 [SMILES: CC(C)n1c(=O)n(C)c2nnc3ccc(-c4ccc([C@@H](C)OCCN5CC6(COC6)C5)nc4)cc3c21] | (R)-8-(6-(1-(2-(2-oxa-6- azaspiro[3.3]heptan-6- yl)ethoxy)ethyl)pyridin-3- yl)-1-isopropyl-3-methyl-1H- imidazo[4,5-c]cinnolin- 2(3H)-one | 489 [M + H]⁺ |
| 82 | 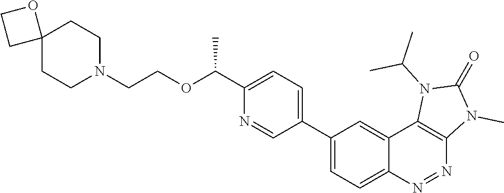 [SMILES: CC(C)n1c(=O)n(C)c2nnc3ccc(-c4ccc([C@@H](C)OCCN5CCC6(CCO6)CC5)nc4)cc3c21] | (R)-8-(6-(1-(2-(1-oxa-7- azaspiro[3.5]nonan-7- yl)ethoxy)ethyl)pyridin-3- yl)-1-isopropyl-3-methyl-1H- imidazo[4,5-c]cinnolin- 2(3H)-one | 517 [M + H]⁺ |
| 83 | 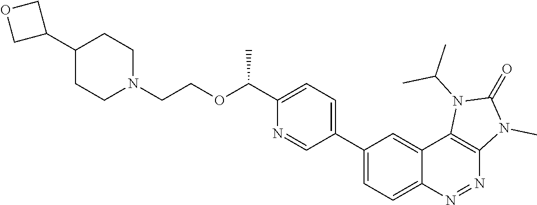 [SMILES: CC(C)n1c(=O)n(C)c2nnc3ccc(-c4ccc([C@@H](C)OCCN5CCC(C6COC6)CC5)nc4)cc3c21] | (R)-1-isopropyl-3-methyl-8- (6-(1-(2-(4-(oxetan-3- yl)piperidin-1- yl)ethoxy)ethyl)pyridin-3-yl)- 1H-imidazo[4,5-c]cinnolin- 2(3H)-one | 531 [M + H]⁺ |
| 84 | 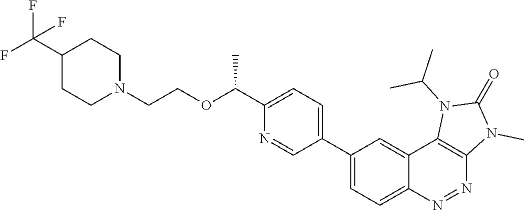 [SMILES: CC(C)n1c(=O)n(C)c2nnc3ccc(-c4ccc([C@@H](C)OCCN5CCC(C(F)(F)F)CC5)nc4)cc3c21] | (R)-1-isopropyl-3-methyl-8- (6-(1-(2-(4-(trifluoromethyl) piperidin-1- yl)ethoxy)ethyl)pyridin-3-yl)- 1H-imidazo[4,5-c]cinnolin- 2(3H)-one | 543 [M + H]⁺ |
| 85 | 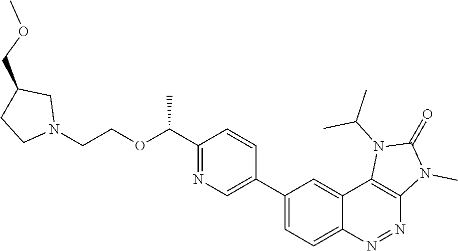 [SMILES: COC[C@@H]1CCN(CCO[C@H](C)c2ccc(-c3ccc4nnc5c(c4c3)n(C(C)C)c(=O)n5C)cn2)C1] | 1-isopropyl-8-(6-((R)-1-(2- ((R)-3- (methoxymethyl)pyrrolidin- 1-yl)ethoxy)ethyl)pyridin-3- yl)-3-methyl-1H-imidazo[4,5- c]cinnolin-2(3H)-one | 505 [M + H]⁺ |
| 86 | 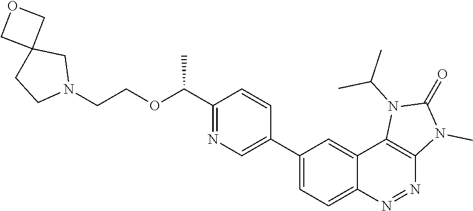 [SMILES: CC(C)n1c(=O)n(C)c2nnc3ccc(-c4ccc([C@@H](C)OCCN5CCC6(COC6)C5)nc4)cc3c21] | (R)-8-(6-(1-(2-(2-oxa-6- azaspiro[3.4]octan-6- yl)ethoxy)ethyl)pyridin-3- yl)-1-isopropyl-3-methyl-1H- imidazo[4,5-c]cinnolin- 2(3H)-one | 503 [M + H]⁺ |
| 87 | 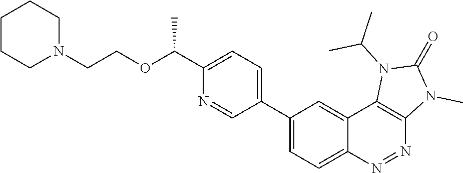 [SMILES: CC(C)n1c(=O)n(C)c2nnc3ccc(-c4ccc([C@@H](C)OCCN5CCCCC5)nc4)cc3c21] | (R)-1-isopropyl-3-methyl-8- (6-(1-(2-(piperidin-1- yl)ethoxy)ethyl)pyridin-3- yl)-1H-imidazo[4,5- c]cinnolin-2(3H)-one | 475 [M + H]⁺ |
| 88 | 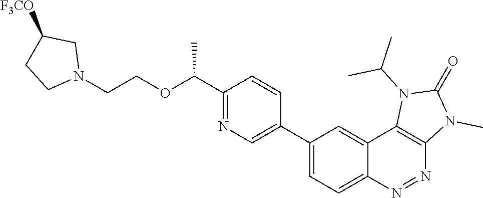 [SMILES: CC(C)n1c(=O)n(C)c2nnc3ccc(-c4ccc([C@@H](C)OCCN5CC[C@@H](OC(F)(F)F)C5)nc4)cc3c21] | 1-isopropyl-3-methyl-8-(6- ((R)-1-(2-((R)-3- (trifluoromethoxy)pyrrolidin- 1-yl)ethoxy)ethyl)pyridin-3- yl)-1H-imidazo[4,5- c]cinnolin-2(3H)-one | 545 [M + H]⁺ |
| 89 | 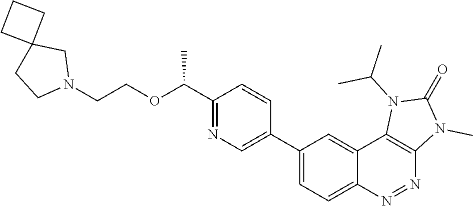 [SMILES: CC(C)n1c(=O)n(C)c2nnc3ccc(-c4ccc([C@@H](C)OCCN5CCC6(CCC6)C5)nc4)cc3c21] | (R)-8-(6-(1-(2-(6- azaspiro[3.4]octan-6- yl)ethoxy)ethyl)pyridin-3-yl)- 1-isopropyl-3-methyl-1H- imidazo[4,5-c]cinnolin- 2(3H)-one | 501 [M + H]⁺ |
| 90 | 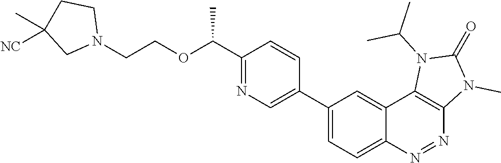 | 1-(2-((R)-1-(5-(1-isopropyl- 3-methyl-2-oxo-2,3-dihydro- 1H-imidazo[4,5-c]cinnolin-8- yl)pyridin-2-yl)ethoxy)ethyl)- 3-methylpyrrolidine-3- carbonitrile | 500 [M + H]⁺ |
| 91 | 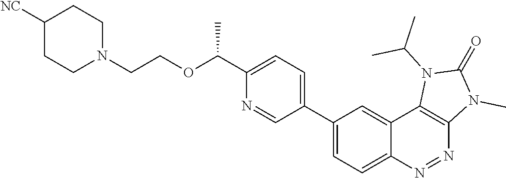 [SMILES: CC(C)n1c(=O)n(C)c2nnc3ccc(-c4ccc([C@@H](C)OCCN5CCC(C#N)CC5)nc4)cc3c21] | (R)-1-(2-(1-(5-(1-isopropyl- 3-methyl-2-oxo-2,3-dihydro- 1H-imidazo[4,5-c]cinnolin-8- yl)pyridin-2- yl)ethoxy)ethyl)piperidine-4- carbonitrile | 500 [M + H]⁺ |
| 92 | 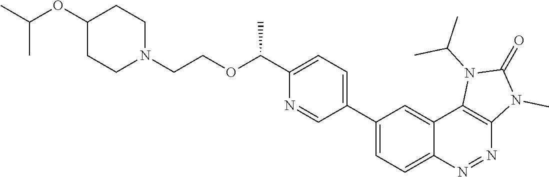 [SMILES: CC(C)OC1CCN(CCO[C@H](C)c2ccc(-c3ccc4nnc5c(c4c3)n(C(C)C)c(=O)n5C)cn2)CC1] | (R)-8-(6-(1-(2-(4- isopropoxypiperidin-1- yl)ethoxy)ethyl)pyridin-3-yl)- 1-isopropyl-3-methyl-1H- imidazo[4,5-c]cinnolin- 2(3H)-one | 533 [M + H]⁺ |
| 93 | 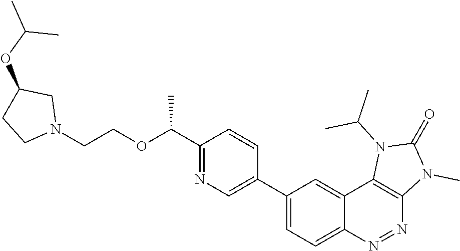 [SMILES: CC(C)O[C@@H]1CCN(CCO[C@H](C)c2ccc(-c3ccc4nnc5c(c4c3)n(C(C)C)c(=O)n5C)cn2)C1] | 8-(6-((R)-1-(2-((R)-3- isopropoxypyrrolidin-1- yl)ethoxy)ethyl)pyridin-3- yl)-1-isopropyl-3-methyl-1H- imidazo[4,5-c]cinnolin- 2(3H)-one | 519 [M + H]⁺ |

### Example 77

¹H NMR (400 MHz, CDCl₃) δ 8.92 (s, 1H), 8.54 (d, J=9.0 Hz, 1H), 8.28 (s, 1H), 8.05 (d, J=7.7 Hz, 1H), 7.87 (d, J=9.1 Hz, 1H), 7.60 (d, J=7.6 Hz, 1H), 5.30-5.14 (m, 1H), 4.75-4.68 (m, 1H), 4.04-3.86 (m, 3H), 3.77 (s, 3H), 3.35-3.26 (m, 2H), 3.22-2.90 (m, 2H), 2.72-2.15 (m, 4H), 1.79 (d, J=6.9 Hz, 6H), 1.58 (d, J=6.4 Hz, 3H), 1.14 (d, J=6.1 Hz, 3H).

### Example 78

¹H NMR (600 MHz, DMSO-d₆) δ 9.10 (d, J=2.0 Hz, 1H), 8.51 (s, 1H), 8.45 (d, J=9.1 Hz, 1H), 8.38 (dd, J=8.1, 2.3 Hz, 1H), 8.17 (dd, J=9.1, 1.4 Hz, 1H), 7.70 (d, J=8.1 Hz, 1H), 5.40-5.32 (m, 1H), 4.74-4.70 (m, 1H), 3.78-3.71 (m, 1H), 3.67-3.62 (m, 2H), 3.60 (s, 3H), 3.45-3.22 (m, 3H), 3.18-3.07 (m, 1H), 2.73-2.63 (m, 1H), 2.37-2.29 (m, 1H), 2.23-2.07 (m, 1H), 1.68 (d, J=6.7 Hz, 6H), 1.66-1.58 (m, 1H), 1.51 (d, J=6.5 Hz, 3H), 1.07 (d, J=6.7 Hz, 3H).

### Example 79

¹H NMR (400 MHz, DMSO-d₆) δ 9.10 (d, J=1.8 Hz, 1H), 8.51 (s, 1H), 8.44 (d, J=9.1 Hz, 1H), 8.37 (dd, J=8.2, 2.1 Hz, 1H), 8.17 (d, J=9.1 Hz, 1H), 7.69 (d, J=8.2 Hz, 1H), 5.44-5.25 (m, 1H), 4.73 (q, J=6.4 Hz, 1H), 4.26-4.13 (m, 1H), 3.80-3.74 (m, 4H), 3.69-3.63 (m, 2H), 3.59 (s, 3H), 3.52-3.46 (m, 2H), 3.15 (s, 3H), 2.46-2.36 (m, 2H), 1.67 (d, J=6.7 Hz, 6H), 1.50 (d, J=6.5 Hz, 3H).

### Example 80

¹H NMR (400 MHz, CDCl₃) δ 8.92 (d, J=2.0 Hz, 1H), 8.56 (d, J=9.0 Hz, 1H), 8.29 (s, 1H), 8.05 (dd, J=8.1, 2.3 Hz, 1H), 7.88 (dd, J=9.0, 1.7 Hz, 1H), 7.61 (d, J=8.1 Hz, 1H), 5.29-5.18 (m, 1H), 4.69 (q, J=6.3 Hz, 1H), 3.93-3.59 (m, 7H), 3.34-3.24 (m, 1H), 3.16-2.88 (m, 4H), 2.22 (2, 2H), 1.98-1.83 (m, 3H), 1.80 (d, J=7.0 Hz, 6H), 1.57 (d, J=6.5 Hz, 3H), 0.62-0.47 (m, 4H).

### Example 81

¹H NMR (400 MHz, CDCl₃) δ 8.90 (s, 1H), 8.56 (d, J=8.9 Hz, 1H), 8.27 (s, 1H), 8.03 (d, J=6.1 Hz, 1H), 7.88 (d, J=9.0 Hz, 1H), 7.62 (d, J=8.1 Hz, 1H), 5.29-5.16 (m, 1H), 4.80-4.70 (m, 4H), 4.61 (q, J=6.6 Hz, 1H), 3.78 (s, 3H), 3.54-3.38 (m, 6H), 2.71-2.58 (m, 2H), 1.80 (d, J=6.9 Hz, 6H), 1.54 (d, J=6.5 Hz, 3H).

### Example 82

¹H NMR (400 MHz, DMSO-d₆) δ 9.05 (d, J=2.0 Hz, 1H), 8.49-8.40 (m, 2H), 8.33 (dd, J=8.2, 2.3 Hz, 1H), 8.10 (dd, J=9.1, 1.4 Hz, 1H), 7.64 (d, J=8.2 Hz, 1H), 5.38-5.26 (m, 1H), 4.59 (q, J=6.4 Hz, 1H), 4.35 (t, J=7.7 Hz, 2H), 3.60 (s, 3H), 3.56-3.53 (m, 1H), 3.47-3.41 (m, 1H), 2.58-2.52 (m, 2H), 2.49-2.44 (m, 2H), 2.33-2.19 (m, 4H), 1.83-1.60 (m, 10H), 1.43 (d, J=6.5 Hz, 3H).

### Example 83

¹H NMR (400 MHz, DMSO-d₆) δ 9.05 (d, J=1.9 Hz, 1H), 8.50-8.41 (m, 2H), 8.33 (dd, J=8.2, 2.4 Hz, 1H), 8.11 (dd, J=9.1, 1.7 Hz, 1H), 7.64 (d, J=8.1 Hz, 1H), 5.38-5.29 (m, 1H), 4.62-4.53 (m, 3H), 4.31 (t, J=6.2 Hz, 2H), 3.60 (s, 3H), 3.56-3.53 (m, 1H), 3.49-3.46 (m, 1H), 2.90-2.83 (m, 2H), 2.71-2.63 (m, 1H), 2.59-2.52 (m, 2H), 2.03-1.92 (m, 2H), 1.67 (d, J=6.7 Hz, 6H), 1.59-1.49 (m, 3H), 1.43 (d, J=6.5 Hz, 3H), 1.08-0.96 (m, 2H).

### Example 84

¹H NMR (400 MHz, DMSO-d₆) δ 9.04 (d, J=2.3 Hz, 1H), 8.49-8.40 (m, 2H), 8.34-8.28 (m, 1H), 8.13-8.05 (m, 1H), 7.63 (d, J=8.1 Hz, 1H), 5.35-5.26 (m, 1H), 4.59 (q, J=6.4 Hz, 1H), 3.60 (s, 3H), 3.57-3.52 (m, 1H), 3.49-3.44 (m, 1H), 3.01-2.89 (m, 2H), 2.60-2.51 (m, 2H), 2.30-2.15 (m, 1H), 2.06-1.94 (m, 2H), 1.79-1.71 (m, 2H), 1.66 (d, J=6.7 Hz, 6H), 1.49-1.39 (m, 5H).

### Example 85

¹H NMR (400 MHz, CDCl₃) δ 8.92 (s, 1H), 8.54 (d, J=9.0 Hz, 1H), 8.27 (s, 1H), 8.05 (d, J=7.6 Hz, 1H), 7.87 (d, J=9.2 Hz, 1H), 7.62 (d, J=7.4 Hz, 1H), 5.28-5.16 (m, 1H), 4.72 (d, J=6.0 Hz, 1H), 3.99-3.83 (m, 2H), 3.76 (s, 3H), 3.73-3.47 (m, 2H), 3.46-3.40 (m, 2H), 3.35 (s, 3H), 3.31-3.17 (m, 3H), 2.83-2.69 (m, 1H), 2.33-2.21 (m, 1H), 1.99-1.73 (m, 8H), 1.58 (d, J=5.9 Hz, 3H).

### Example 86

¹H NMR (400 MHz, CDCl₃) δ 8.92 (s, 1H), 8.55 (d, J=9.0 Hz, 1H), 8.28 (s, 1H), 8.04 (d, J=8.2 Hz, 1H), 7.87 (d, J=9.0 Hz, 1H), 7.59 (d, J=8.1 Hz, 1H), 5.29-5.17 (m, 1H), 5.14-4.98 (m, 1H), 4.72-4.62 (m, 4H), 3.83-3.64 (m, 5H), 3.39-3.26 (m, 2H), 3.10-2.96 (m, 4H), 2.31 (t, J=7.0 Hz, 2H), 1.79 (d, J=6.9 Hz, 6H), 1.57 (d, J=6.4 Hz, 3H).

### Example 87

¹H NMR (400 MHz, CDCl₃) δ 8.92 (s, 1H), 8.56 (d, J=8.9 Hz, 1H), 8.28 (s, 1H), 8.04 (d, J=6.5 Hz, 1H), 7.88 (d, J=9.1 Hz, 1H), 7.59 (d, J=8.1 Hz, 1H), 5.28-5.15 (m, 1H), 4.68 (q, J=6.5 Hz, 1H), 3.93-3.75 (m, 5H), 3.10-2.85 (m, 6H), 1.93-1.84 (m, 4H), 1.79 (d, J=6.9 Hz, 6H), 1.63-1.52 (m, 5H).

### Example 88

¹H NMR (400 MHz, DMSO-d₆) δ 9.05 (d, J=2.1 Hz, 1H), 8.50-8.42 (m, 2H), 8.31 (dd, J=8.1, 2.3 Hz, 1H), 8.10 (d, J=9.1 Hz, 1H), 7.64 (d, J=8.1 Hz, 1H), 5.38-5.28 (m, 1H), 4.96-4.89 (m, 1H), 4.65-4.56 (m, 1H), 3.61 (s, 3H), 3.59-3.53 (m, 1H), 3.49-3.45 (m, 1H), 2.81-2.60 (m, 5H), 2.39-2.32 (m, 1H), 2.27-2.19 (m, 1H), 1.90-1.79 (m, 1H), 1.67 (d, J=6.7 Hz, 6H), 1.44 (d, J=6.5 Hz, 3H).

### Example 89

¹H NMR (400 MHz, CDCl₃) δ 8.92 (s, 1H), 8.56 (d, J=9.0 Hz, 1H), 8.47 (s, 1H), 8.04 (d, J=7.2 Hz, 1H), 7.88 (d, J=9.1 Hz, 1H), 7.59 (d, J=8.2 Hz, 1H), 5.31-5.14 (m, 1H), 4.74-4.65 (m, 1H), 3.91-3.72 (m, 5H), 3.37-3.11 (m, 6H), 2.25-1.84 (m, 8H), 1.79 (d, J=6.9 Hz, 6H), 1.57 (d, J=6.4 Hz, 3H).

### Example 90

¹H NMR (400 MHz, DMSO-d₆) δ 9.05 (d, J=2.1 Hz, 1H), 8.51-8.40 (m, 2H), 8.36-8.29 (m, 1H), 8.10 (dd, J=9.1, 1.5 Hz, 1H), 7.66 (dd, J=8.1, 4.8 Hz, 1H), 5.37-5.26 (m, 1H), 4.61 (q, J=6.5 Hz, 1H), 3.60 (s, 3H), 3.58-3.55 (m, 1H), 3.48-3.43 (m, 1H), 3.10-3.02 (m, 1H), 2.86-2.76 (m, 1H), 2.74-2.53 (m, 3H), 2.46-2.37 (m, 1H), 2.28-2.17 (m, 1H), 1.86-1.76 (m, 1H), 1.67 (d, J=6.7 Hz, 6H), 1.45 (d, J=6.5 Hz, 3H), 1.40 (d, J=2.2 Hz, 3H).

### Example 91

¹H NMR (400 MHz, DMSO-d₆) δ 9.03 (s, 1H), 8.47-8.39 (m, 2H), 8.31 (d, J=8.2 Hz, 1H), 8.08 (d, J=9.1 Hz, 1H), 7.62 (d, J=8.2 Hz, 1H), 5.38-5.24 (m, 1H), 4.57 (q, J=6.4 Hz, 1H), 3.58 (s, 3H), 3.56-3.50 (m, 2H), 2.86-2.79 (m, 1H), 2.62-2.51 (m, 4H), 2.37-2.26 (m, 2H), 1.86-1.77 (m, 2H), 1.72-1.60 (m, 8H), 1.42 (d, J=6.5 Hz, 3H).

### Example 92

¹H NMR (400 MHz, CDCl₃) δ 8.91 (s, 1H), 8.55 (d, J=9.1 Hz, 1H), 8.28 (s, 1H), 8.03 (d, J=8.2 Hz, 1H), 7.88 (d, J=8.7 Hz, 1H), 7.62 (d, J=7.8 Hz, 1H), 5.27-5.18 (m, 1H), 4.72-4.61 (m, 1H), 4.45-4.26 (m, 1H), 3.85-3.62 (m, 7H), 3.58-3.49 (m, 1H), 3.08-2.79 (m, 4H), 2.15-1.96 (m, 4H), 1.79 (d, J=6.7 Hz, 6H), 1.56 (d, J=6.0 Hz, 3H), 1.15 (d, J=5.6 Hz, 6H).

### Example 93

¹H NMR (400 MHz, CDCl₃) δ 9.02 (s, 1H), 8.55 (d, J=9.2 Hz, 1H), 8.31 (s, 1H), 8.14 (d, J=8.8 Hz, 1H), 7.89 (d, J=9.1 Hz, 1H), 7.72 (s, 1H), 5.28-5.15 (m, 1H), 4.92-4.77 (m, 1H), 4.41-4.27 (m, 1H), 4.17-3.90 (m, 4H), 3.77 (s, 3H), 3.70-3.59 (m, 1H), 3.54-3.34 (m, 2H), 3.27-3.10 (m, 2H), 2.40-2.22 (m, 1H), 2.18-2.05 (m, 1H), 1.80 (d, J=6.4 Hz, 6H), 1.62 (d, J=6.1 Hz, 3H), 1.16 (d, J=5.8 Hz, 6H).

### Example 94

### 8-(6-((2-(5-azaspiro[2.4]heptan-5-yl)ethoxy)methyl)pyridin-3-yl)-3-methyl-1-(tetrahydro-2H-pyran-4-yl)-1H-imidazo[4,5-c]cinnolin-2(3H)-one

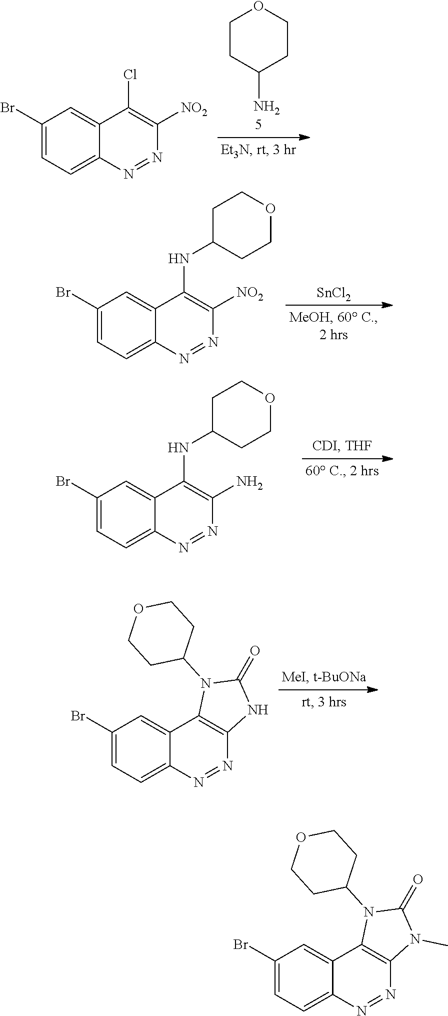

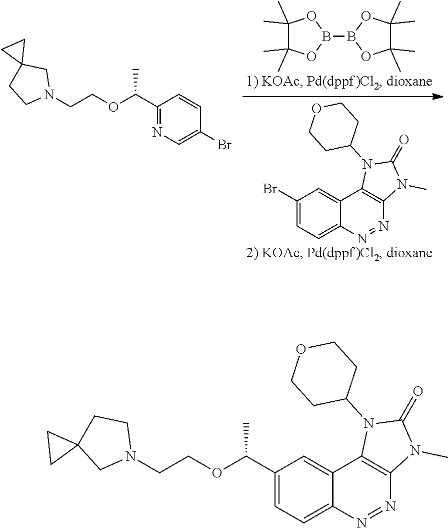

### Step 1: 6-bromo-3-nitro-N-(tetrahydro-2H-pyran-4-yl)cinnolin-4-amine

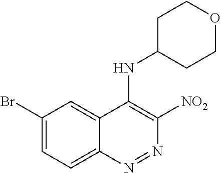 [SMILES: O=[N+]([O-])c1nnc2ccc(Br)cc2c1NC1CCOCC1]

To a mixture of 6-bromo-4-chloro-3-nitrocinnoline (60.0 g, 170 mmol) and tetrahydro-2H-pyran-4-amine (25.8 g, 255 mmol) in DCM (500 mL) was added TEA (75.4 mL, 510 mmol) and the reaction mixture was stirred at room temperature for 3 h. The mixture was poured into ice 1 M HCl solution (1 L) and extracted with DCM (500 mL×2). The combined organic layers were washed with brine (800 mL), dried over anhydrous Na₂SO₄ and filtered, the filtrate was concentrated under reduced pressure to give the desired product (75.0 g, 100.0% yield) as a brown solid. LC-MS (ESI) m z: 353 [M+H]⁺.

### Step 2: 6-bromo-N4-(tetrahydro-2H-pyran-4-yl)cinnoline-3,4-diamine

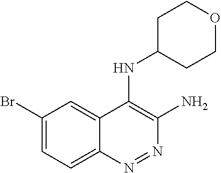 [SMILES: Nc1nnc2ccc(Br)cc2c1NC1CCOCC1]

To a solution of 6-bromo-3-nitro-N-(tetrahydro-2H-pyran-4-yl)cinnolin-4-amine (75.0 g, 212 mmol) in MeOH (200 mL) was added SnCl₂—H₂O (144 g, 636 mmol) at 0° C. and the reaction mixture was stirred at 60° C. for 2 h. The mixture was adjusted with 50% NaOH solution until pH˜8 below 45° C. and stirred at 55° C. for 45 min. The mixture was filtered and the filtered residue was trituration with THE (300 mL) for 1 h at room temperature. It was filtered and the combined filtrate was evaporated under reduced pressure. The residue was triturated with THF (300 mL) for 1 h at room temperature and filtered. The filtrate was evaporated under reduced pressure to give the desired product (54.0 g, 78.7% yield) as a dark brown solid. LC-MS (ESI) m z: 323 [M+H]⁺.

### Step 3: 8-bromo-1-(tetrahydro-2H-pyran-4-yl)-1H-imidazo[4,5-c]cinnolin-2(3H)-one

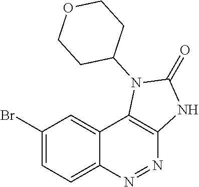 [SMILES: O=c1[nH]c2nnc3ccc(Br)cc3c2n1C1CCOCC1]

To a solution of 6-bromo-N4-(tetrahydro-2H-pyran-4-yl)cinnoline-3,4-diamine (54.0 g, 154 mmol) in dry THF (500 mL) was added CDI (75.0 g, 462 mmol) and the reaction mixture was stirred at room temperature for 2 h. The mixture was evaporated under reduced pressure and the residue was dissolved in water (250 mL). It was adjusted with 1 M HCl solution until pH˜7 and filtered. The filtered residue was dried in oven at 55° C. for 16 h to give the desired product (42.0 g, 72.0% yield). LC-MS (ESI) m z: 349 [M+H]⁺.

### Step 4: 8-bromo-3-methyl-1-(tetrahydro-2H-pyran-4-yl)-1H-imidazo[4,5-c]cinnolin-2(3H)-one

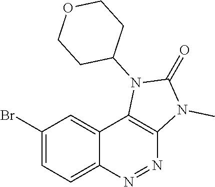 [SMILES: Cn1c(=O)n(C2CCOCC2)c2c3cc(Br)ccc3nnc21]

To a solution of 8-bromo-1-(tetrahydro-2H-pyran-4-yl)-1H-imidazo[4,5-c]cinnolin-2(3H)-one (42.0 g, 120 mmol) in dry DMF (300 mL) was added t-BuONa (14.0 g, 144 mmol) at 0° C. and stirred at 0° C. for 30 min under N₂. Then to the reaction mixture was added dropwise Mel (34.1 g, 240 mmol) at 10° C.-15° C. and the reaction mixture was stirred at 10° C.-15° C. for 2 h. The mixture was poured into ice water (600 mL) and filtered. The filtered residue was triturated with MeOH (200 mL) for 1 h and filtered. The filter cake was dried under vacuum to give the desired product (35.0 g, 80.0% yield). LC-MS (ESI) m z: 363 [M+H]⁺.

### Step 5: 8-(6-((2-(5-azaspiro[2.4]heptan-5-yl)ethoxy)methyl)pyridin-3-yl)-3-methyl-1-(tetrahydro-2H-pyran-4-yl)-1H-imidazo[4,5-c]cinnolin-2(3H)-one

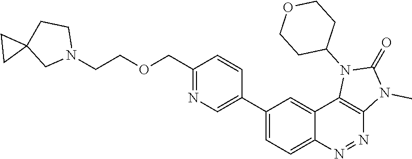 [SMILES: Cn1c(=O)n(C2CCOCC2)c2c3cc(-c4ccc(COCCN5CCC6(CC6)C5)nc4)ccc3nnc21]

The crude product was prepared in a similar fashion to Example 1, which was purified by flash chromatography (DCM:MeOH=9:1) to give the desired product (40.0 mg, 24.1% yield) as a light brown solid. ¹H NMR (400 MHz, DMSO-d₆) δ 9.07 (d, J=1.9 Hz, 1H), 8.53-8.43 (m, 2H), 8.35 (dd, J=8.2, 2.4 Hz, 1H), 8.14 (dd, J=9.1, 1.6 Hz, 1H), 7.65 (d, J=8.1 Hz, 1H), 5.18-5.04 (m, 1H), 4.68 (s, 2H), 4.11-4.01 (m, 2H), 3.70 (t, J=5.8 Hz, 2H), 3.65-3.60 (m, 5H), 2.85-2.77 (m, 4H), 2.72-2.59 (m, 4H), 2.00-1.92 (m, 2H), 1.76 (t, J=6.9 Hz, 2H), 0.59-0.48 (in, 4H). LC-MS (ESI) m/z: 515 [M+H]⁺.

The following compounds were prepared according to the above described methods using different starting materials.

| | | | MS |
|---|---|---|---|
| Ex# | Structure | Name | m/z |
| 95 | 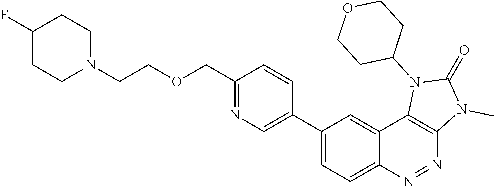 [SMILES: Cn1c(=O)n(C2CCOCC2)c2c3cc(-c4ccc(COCCN5CCC(F)CC5)nc4)ccc3nnc21] | 8-(6-((2-(4-fluoropiperidin-1- yl)ethoxy)methyl)pyridin-3- yl)-3-methyl-1-(tetrahydro- 2H-pyran-4-yl)-1H- imidazo[4,5-c]cinnolin- 2(3H)-one | 521 [M + H]⁺ |
| 96 | 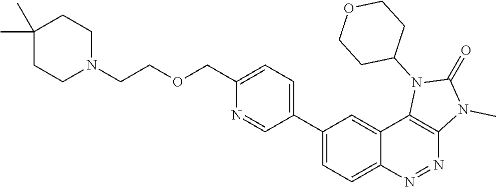 | 8-(6-((2-(4,4- dimethylpiperidin-1- yl)ethoxy)methyl)pyridin-3- yl)-3-methyl-1-(tetrahydro- 2H-pyran-4-yl)-1H- imidazo[4,5-c]cinnolin- 2(3H)-one | 531 [M + H]⁺ |
| 97 | 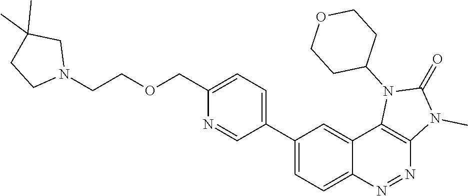 | 8-(6-((2-(3,3- dimethylpyrrolidin-1- yl)ethoxy)methyl)pyridin-3- yl)-3-methyl-1-(tetrahydro- 2H-pyran-4-yl)-1H- imidazo[4,5-c]cinnolin- 2(3H)-one | 517 [M + H]⁺ |
| 98 | 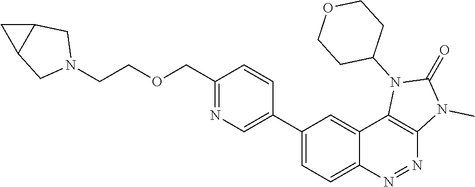 [SMILES: Cn1c(=O)n(C2CCOCC2)c2c3cc(-c4ccc(COCCN5CC6CC6C5)nc4)ccc3nnc21] | 8-(6-((2-(3- azabicyclo[3.1.0]hexan-3- yl)ethoxy)methyl)pyridin-3- yl)-3-methyl-1-(tetrahydro- 2H-pyran-4-yl)-1H- imidazo[4,5-c]cinnolin- 2(3H)-one | 501 [M + H]⁺ |
| 99 | 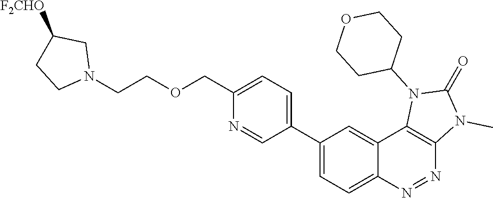 | (R)-8-(6-((2-(3- (difluoromethoxy)pyrrolidin- 1-yl)ethoxy)methyl)pyridin- 3-yl)-3-methyl-1-(tetrahydro- 2H-pyran-4-yl)-1H- imidazo[4,5-c]cinnolin- 2(3H)-one | 555 [M + H]⁺ |
| 100 | 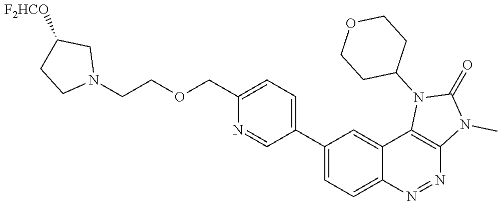 [SMILES: Cn1c(=O)n(C2CCOCC2)c2c3cc(-c4ccc(COCCN5CC[C@H](OC(F)F)C5)nc4)ccc3nnc21] | (S)-8-(6-((2-(3- (difluoromethoxy)pyrrolidin- 1-yl)ethoxy)methyl)pyridin- 3-yl)-3-methyl-1-(tetrahydro- 2H-pyran-4-yl)-1H- imidazo[4,5-c]cinnolin- 2(3H)-one | 555 [M + H]⁺ |
| 101 | 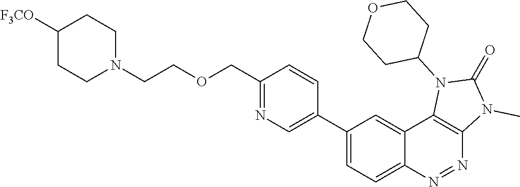 [SMILES: Cn1c(=O)n(C2CCOCC2)c2c3cc(-c4ccc(COCCN5CCC(OC(F)(F)F)CC5)nc4)ccc3nnc21] | 3-methyl-1-(tetrahydro-2H- pyran-4-yl)-8-(6-((2-(4- (trifluoromethoxy)piperidin- 1-yl)ethoxy)methyl)pyridin- 3-yl)-1H-imidazo[4,5- c]cinnolin-2(3H)-one | 587 [M + H]⁺ |

### Example 95

¹H NMR (400 MHz, DMSO-d₆) δ 9.06 (s, 1H), 8.50 (s, 1H), 8.45 (d, J=9.2 Hz, 1H), 8.33 (d, J=8.1 Hz, 1H), 8.13 (d, J=9.1 Hz, 1H), 7.64 (d, J=8.1 Hz, 1H), 5.16-5.05 (m, 1H), 4.77-4.55 (m, 3H), 4.10-3.99 (m, 2H), 3.70-3.56 (m, 7H), 2.71-2.55 (m, 6H), 2.41-2.31 (m, 2H), 2.00-1.92 (m, 2H), 1.92-1.78 (m, 2H), 1.75-1.64 (m, 2H).

### Example 96

¹H NMR (400 MHz, DMSO-d₆) δ 9.06 (d, J=2.0 Hz, 1H), 8.51 (s, 1H), 8.46 (d, J=9.1 Hz, 1H), 8.34 (dd, J=8.2, 2.4 Hz, 1H), 8.14 (dd, J=9.1, 1.6 Hz, 1H), 7.64 (d, J=8.1 Hz, 1H), 5.17-5.07 (m, 1H), 4.66 (s, 2H), 4.06 (dd, J=11.2, 4.4 Hz, 2H), 3.69-3.58 (m, 7H), 2.72-2.62 (m, 2H), 2.58 (t, J=5.9 Hz, 2H), 2.44-2.37 (m, 4H), 2.00-1.93 (m, 2H), 1.34-1.30 (m, 4H), 0.89 (s, 6H).

### Example 97

¹H NMR (400 MHz, CDCl₃) δ 8.98 (s, 1H), 8.59 (d, J=8.9 Hz, 1H), 8.47 (s, 1H), 8.11 (d, J=7.6 Hz, 1H), 7.95 (d, J=9.0 Hz, 1H), 7.67 (d, J=8.2 Hz, 1H), 5.17-5.03 (m, 1H), 4.80 (s, 2H), 4.33-4.18 (m, 2H), 3.98-3.86 (m, 2H), 3.82 (s, 3H), 3.65 (t, J=11.9 Hz, 2H), 3.17-3.01 (m, 4H), 2.94-2.72 (m, 4H), 2.04-1.94 (m, 2H), 1.79 (t, J=6.5 Hz, 2H), 1.20 (s, 6H).

### Example 98

¹H NMR (400 MHz, DMSO-d₆) δ 9.06 (d, J=2.3 Hz, 1H), 8.50 (s, 1H), 8.45 (d, J=9.1 Hz, 1H), 8.33 (dd, J=8.1, 2.3 Hz, 1H), 8.13 (d, J=9.1 Hz, 1H), 7.62 (d, J=8.1 Hz, 1H), 5.16-5.05 (m, 1H), 4.65 (s, 2H), 4.09-3.99 (m, 2H), 3.66-3.53 (m, 7H), 3.02-2.94 (m, 2H), 2.72-2.60 (m, 4H), 2.37-2.30 (m, 2H), 2.00-1.92 (m, 2H), 1.40-1.25 (m, 2H), 0.61-0.56 (m, 1H), 0.34-0.25 (m, 1H).

### Example 99

¹H NMR (400 MHz, DMSO-d₆) δ 9.05 (s, 1H), 8.53-8.38 (m, 2H), 8.32 (d, J=8.1 Hz, 1H), 8.13 (s, 1H), 7.62 (d, J=7.9 Hz, 1H), 6.65 (t, J=76.0 Hz, 1H), 5.14-5.04 (m, 1H), 4.72-4.62 (m, 3H), 4.08-3.98 (m, 2H), 3.70-3.55 (m, 7H), 2.82-2.57 (m, 7H), 2.45-2.40 (m, 1H), 2.18-2.08 (m, 1H), 1.99-1.89 (m, 2H), 1.81-1.70 (m, 1H).

### Example 100

¹H NMR (400 MHz, DMSO-d₆)) δ 9.04 (s, 1H), 8.51-8.40 (m, 2H), 8.31 (d, J=8.2 Hz, 1H), 8.13 (s, 1H), 7.62 (d, J=8.2 Hz, 1H), 6.64 (t, J=75.9 Hz, 1H), 5.13-5.03 (m, 1H), 4.71-4.60 (m, 3H), 4.08-3.99 (m, 2H), 3.68-3.53 (m, 7H), 2.79-2.55 (m, 7H), 2.45-2.38 (m, 1H), 2.18-2.07 (m, 1H), 1.98-1.89 (m, 2H), 1.79-1.69 (m, 1H).

### Example 101

¹H NMR (400 MHz, DMSO-d₆) δ 9.05 (s, 1H), 8.53-8.41 (m, 2H), 8.33 (d, J=7.4 Hz, 1H), 8.14 (s, 1H), 7.63 (d, J=7.8 Hz, 1H), 5.17-5.03 (m, 1H), 4.65 (s, 2H), 4.47-4.35 (m, 1H), 4.10-3.99 (m, 2H), 3.69-3.55 (m, 7H), 2.77-2.55 (m, 6H), 2.35-2.26 (m, 2H), 2.00-1.84 (m, 4H), 1.74-1.62 (m, 2H).

### Example 102

### (R)-8-(6-(1-(2-(5-azaspiro[2.4]heptan-5-yl)ethoxy)ethyl)pyridin-3-yl)-3-methyl-1-(tetrahydro-2H-pyran-4-yl)-1H-imidazo[4,5-c]cinnolin-2(3H)-one

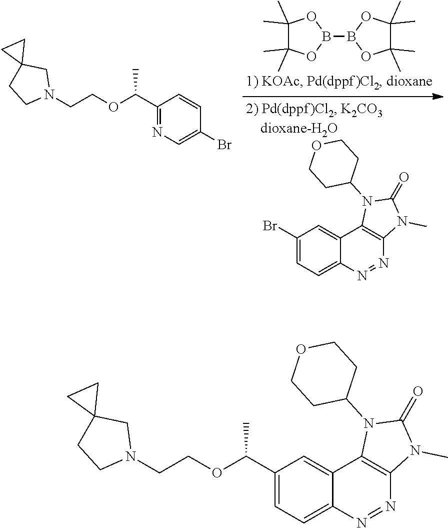

### Step 1: (R)-8-(6-(1-(2-(5-azaspiro[2.4]heptan-5-yl)ethoxy)ethyl)pyridin-3-yl)-3-methyl-1-(tetrahydro-2H-pyran-4-yl)-1H-imidazo[4,5-c]cinnolin-2(3H)-one

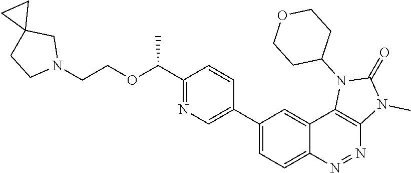 [SMILES: C[C@@H](OCCN1CCC2(CC2)C1)c1ccc(-c2ccc3nnc4c(c3c2)n(C2CCOCC2)c(=O)n4C)cn1]

The crude product was prepared in a similar fashion to Example 1, which was purified by chromatography on silica gel (DCM:MeOH=100:1 to 10:1) to give the desired product (4.6 g, 28.3% yield) as an off-white solid. ¹H NMR (400 MHz, CDCl₃) δ 8.95 (s, 1H), 8.56 (d, J=8.8 Hz, 1H), 8.45 (s, 1H), 8.09 (d, J=8.0 Hz, 1H), 7.92 (d, J=9.0 Hz, 1H), 7.58 (d, J=8.0 Hz, 1H), 5.08 (s, 1H), 4.73-4.48 (m, 5H), 4.26 (d, J=8.0 Hz, 2H), 3.90-3.75 (m, 5H), 3.63 (t, J=11.8 Hz, 2H), 3.50-3.37 (m, 2H), 3.31-3.08 (m, 4H), 2.75-2.95 (m, 2H), 1.55 (d, J=6.2 Hz, 3H), 0.70 (d, J=10.4 Hz, 4H). LC-MS (ESI) m z: 529 [M+H]⁺.

The following compounds were prepared according to the above described methods using different starting materials.

| | | | MS |
|---|---|---|---|
| Ex# | Structure | Name | m/z |
| 103 | 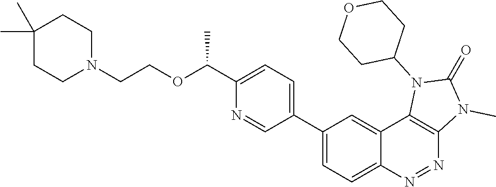 | (R)-8-(6-(1-(2-(4,4- dimethylpiperidin-1- yl)ethoxy)ethyl)pyridin-3-yl)- 3-methyl-1-(tetrahydro-2H- pyran-4-yl)-1H-imidazo[4,5- c]cinnolin-2(3H)-one | 545 [M + H]⁺ |
| 104 | 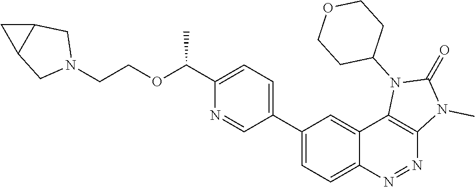 [SMILES: C[C@@H](OCCN1CC2CC2C1)c1ccc(-c2ccc3nnc4c(c3c2)n(C2CCOCC2)c(=O)n4C)cn1] | 8-(6-((1R)-1-(2-(3- azabicyclo[3.1.0]hexan-3- yl)ethoxy)ethyl)pyridin-3-yl)- 3-methyl-1-(tetrahydro-2H- pyran-4-yl)-1H-imidazo[4,5- c]cinnolin-2(3H)-one | 515 [M + H]⁺ |
| 105 | 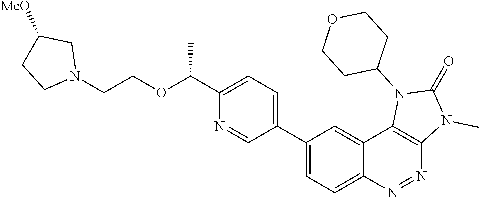 | 8-(6-((R)-1-(2-((S)-3- methoxypyrrolidin-1- yl)ethoxy)ethyl)pyridin-3-yl)- 3-methyl-1-(tetrahydro-2H- pyran-4-yl)-1H-imidazo[4,5- c]cinnolin-2(3H)-one | 501 [M + H]⁺ |
| 106 | 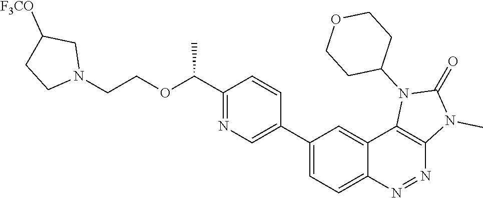 [SMILES: C[C@@H](OCCN1CCC(OC(F)(F)F)C1)c1ccc(-c2ccc3nnc4c(c3c2)n(C2CCOCC2)c(=O)n4C)cn1] | 3-methyl-1-(tetrahydro-2H- pyran-4-yl)-8-(6-((1R)-1-(2- (3- (trifluoromethoxy)pyrrolidin- 1-yl)ethoxy)ethyl)pyridin-3- yl)-1H-imidazo[4,5- c]cinnolin-2(3H)-one | 587 [M + H]⁺ |
| 107 | 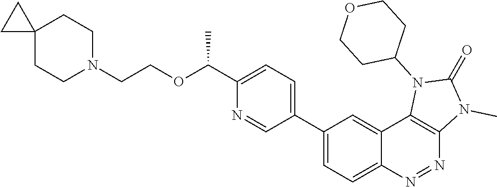 [SMILES: C[C@@H](OCCN1CCC2(CC1)CC2)c1ccc(-c2ccc3nnc4c(c3c2)n(C2CCOCC2)c(=O)n4C)cn1] | (R)-8-(6-(1-(2-(6- azaspiro[2.5]octan-6- yl)ethoxy)ethyl)pyridin-3-yl)- 3-methyl-1-(tetrahydro-2H- pyran-4-yl)-1H-imidazo[4,5- c]cinnolin-2(3H)-one | 543 [M + H]⁺ |
| 108 | 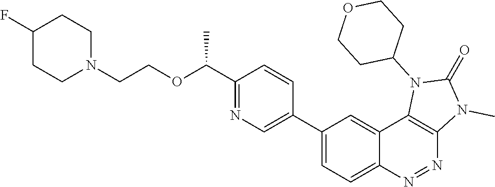 [SMILES: C[C@@H](OCCN1CCC(F)CC1)c1ccc(-c2ccc3nnc4c(c3c2)n(C2CCOCC2)c(=O)n4C)cn1] | 8-(6-((R)-1-(2-(4- fluoropiperidin-1- yl)ethoxy)ethyl)pyridin-3-yl)- 3-methyl-1-(tetrahydro-2H- pyran-4-yl)-1,3,3a,9b- tetrahydro-2H-imidazo[4,5- c]cinnolin-2-one | 535 [M + H]⁺ |
| 109 | 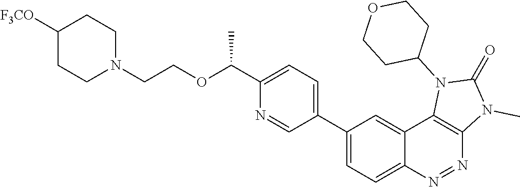 [SMILES: C[C@@H](OCCN1CCC(OC(F)(F)F)CC1)c1ccc(-c2ccc3nnc4c(c3c2)n(C2CCOCC2)c(=O)n4C)cn1] | (R)-3-methyl-1-(tetrahydro- 2H-pyran-4-yl)-8-(6-(1-(2-(4- (trifluoromethoxy)piperidin- 1-yl)ethoxy)ethyl)pyridin-3- yl)-1H-imidazo[4,5- c]cinnolin-2(3H)-one | 601 [M + H]⁺ |
| 110 | 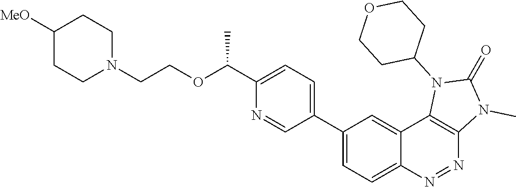 [SMILES: COC1CCN(CCO[C@H](C)c2ccc(-c3ccc4nnc5c(c4c3)n(C3CCOCC3)c(=O)n5C)cn2)CC1] | (R)-8-(6-(1-(2-(4- methoxypiperidin-1- yL)ethoxy)ethyl)pyridin-3-yl)- 3-methyl-1-(tetrahydro-2H- pyran-4-yl)-1H-imidazo[4,5- c]cinnolin-2(3H)-one | 547 [M + H]⁺ |
| 111 | 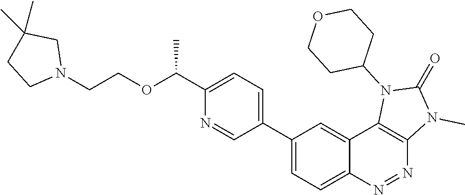 | (R)-8-(6-(1-(2-(3,3- dimethylpyrrolidin-1- yl)ethoxy)ethyl)pyridin-3-yl)- 3-methyl-1-(tetrahydro-2H- pyran-4-yl)-1H-imidazo[4,5- c]cinnolin-2(3H)-one | 531 [M + H]⁺ |
| 112 | 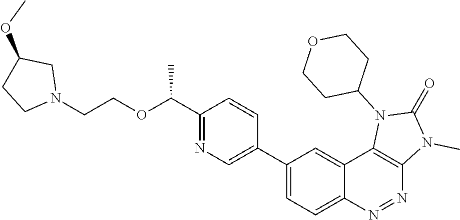 [SMILES: CO[C@@H]1CCN(CCO[C@H](C)c2ccc(-c3ccc4nnc5c(c4c3)n(C3CCOCC3)c(=O)n5C)cn2)C1] | 8-(6-((R)-1-(2-((R)-3- methoxypyrrolidin-1- yl)ethoxy)ethyl)pyridin-3- yl)-3-methyl-1-(tetrahydro- 2H-pyran-4-yl)-1H- imidazo[4,5-c]cinnolin- 2(3H)-one | 533 [M + H]⁺ |
| 113 | 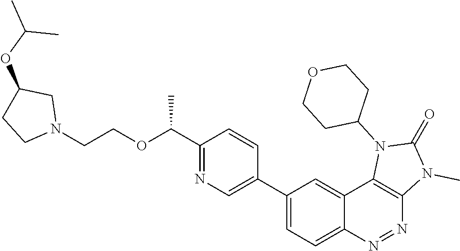 [SMILES: CC(C)O[C@@H]1CCN(CCO[C@H](C)c2ccc(-c3ccc4nnc5c(c4c3)n(C3CCOCC3)c(=O)n5C)cn2)C1] | 8-(6-((R)-1-(2-((R)-3- isopropoxypyrrolidin-1- yl)ethoxy)ethyl)pyridin-3-yl)- 3-methyl-1-(tetrahydro-2H- pyran-4-yl)-1H-imidazo[4,5- c]cinnolin-2(3H)-one | 561 [M + H]⁺ |
| 114 | 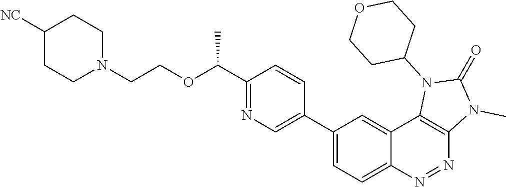 [SMILES: C[C@@H](OCCN1CCC(C#N)CC1)c1ccc(-c2ccc3nnc4c(c3c2)n(C2CCOCC2)c(=O)n4C)cn1] | (R)-1-(2-(1-(5-(3-methyl-2- oxo-1-(tetrahydro-2H-pyran- 4-yl)-2,3-dihydro-1H- imidazo[4,5-c]cinnolin-8- yl)pyridin-2- yl)ethoxy)ethyl)piperidine-4- carbonitrile | 542 [M + H]⁺ |
| 115 | 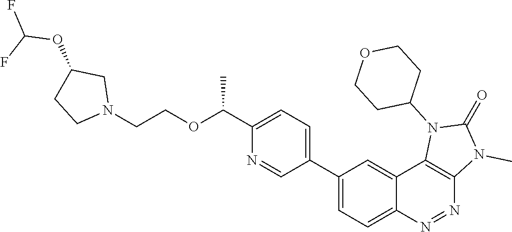 [SMILES: C[C@@H](OCCN1CC[C@H](OC(F)F)C1)c1ccc(-c2ccc3nnc4c(c3c2)n(C2CCOCC2)c(=O)n4C)cn1] | 8-(6-((R)-1-(2-((S)-3- (difluoromethoxy)pyrrolidin- 1-yl)ethoxy)ethyl)pyridin-3- yl)-3-methyl-1-(tetrahydro- 2H-pyran-4-yl)-1,3-dihydro- 2H-imidazo[4,5-c]cinnolin-2- one | 569 [M + H]⁺ |

### Example 103

¹H NMR (400 MHz, DMSO-d₆) δ 9.07 (d, J=2.0 Hz, 1H), 8.52 (s, 1H), 8.45 (d, J=9.1 Hz, 1H), 8.35 (dd, J=8.2, 2.4 Hz, 1H), 8.14 (dd, J=9.1, 1.6 Hz, 1H), 7.66 (d, J=8.2 Hz, 1H), 5.18-5.05 (m, 1H), 4.61 (q, J=6.5 Hz, 1H), 4.10-3.99 (m, 2H), 3.62 (s, 3H), 3.60-3.56 (m, 2H), 3.52-3.45 (m, 2H), 2.72-2.57 (m, 4H), 2.49-2.42 (m, 4H), 2.01-1.91 (m, 2H), 1.44 (d, J=6.5 Hz, 3H), 1.36-1.29 (m, 4H), 0.89 (s, 6H).

### Example 104

¹H NMR (400 MHz, DMSO-d₆) δ 9.07 (d, J=2.3 Hz, 1H), 8.53 (s, 1H), 8.45 (d, J=9.1 Hz, 1H), 8.34 (dd, J=8.2, 2.4 Hz, 1H), 8.15 (dd, J=9.1, 1.7 Hz, 1H), 7.64 (d, J=8.2 Hz, 1H), 5.18-5.07 (m, 1H), 4.59 (q, J=6.5 Hz, 1H), 4.11-4.00 (m, 2H), 3.67-3.58 (m, 5H), 3.55-3.48 (m, 1H), 3.45-3.39 (m, 1H), 3.00-2.91 (m, 2H), 2.74-2.58 (m, 4H), 2.40-2.26 (m, 2H), 2.02-1.91 (m, 2H), 1.43 (d, J=6.5 Hz, 3H), 1.38-1.32 (m, 2H), 0.63-0.56 (m, 1H), 0.35-0.25 (m, 1H).

### Example 105

¹H NMR (400 MHz, DMSO-d₆) δ 9.07 (d, J=1.9 Hz, 1H), 8.53 (s, 1H), 8.46 (d, J=9.1 Hz, 1H), 8.39-8.31 (m, 1H), 8.15 (dd, J=9.1, 1.6 Hz, 1H), 7.65 (d, J=8.2 Hz, 1H), 5.19-5.06 (m, 1H), 4.60 (q, J=6.5 Hz, 1H), 4.09-4.01 (m, 2H), 3.91-3.80 (m, 1H), 3.68-3.59 (m, 5H), 3.58-3.52 (m, 2H), 3.15 (s, 3H), 2.75-2.55 (m, 6H), 2.45-2.37 (m, 2H), 2.01-1.87 (m, 3H), 1.66-1.58 (m, 1H), 1.44 (d, J=6.5 Hz, 3H).

### Example 106

¹H NMR (400 MHz, DMSO-d₆) δ 9.07 (d, J=1.9 Hz, 1H), 8.53 (s, 1H), 8.46 (d, J=9.1 Hz, 1H), 8.38-8.30 (m, 1H), 8.18-8.10 (m, 1H), 7.65 (d, J=8.2 Hz, 1H), 5.18-5.06 (m, 1H), 4.98-4.89 (m, 1H), 4.61 (q, J=6.5 Hz, 1H), 4.13-3.99 (m, 2H), 3.68-3.53 (m, 7H), 2.84-2.59 (m, 7H), 2.41-2.31 (m, 1H), 2.28-2.18 (m, 1H), 2.00-1.92 (m, 2H), 1.89-1.79 (m, 1H), 1.45 (d, J=6.5 Hz, 3H).

### Example 107

¹H NMR (400 MHz, CDCl₃) δ 8.94 (s, 1H), 8.57 (d, J=9.1 Hz, 1H), 8.43 (s, 1H), 8.07 (d, J=8.2 Hz, 1H), 7.92 (d, J=8.9 Hz, 1H), 7.66 (d, J=8.1 Hz, 1H), 5.11-5.00 (m, 1H), 4.72-4.57 (m, 1H), 4.33-4.18 (m, 2H), 3.81-3.57 (m, 7H), 2.94-2.59 (m, 9H), 2.02-1.92 (m, 2H), 1.60-1.38 (m, 7H), 0.29 (s, 4H).

### Example 108

¹H NMR (400 MHz, DMSO-d₆) δ 9.05 (s, 1H), 8.50 (s, 1H), 8.43 (d, J=9.0 Hz, 1H), 8.33 (d, J=8.1 Hz, 1H), 8.12 (d, J=9.0 Hz, 1H), 7.65 (d, J=8.2 Hz, 1H), 5.17-5.04 (m, 1H), 4.75-4.53 (m, 2H), 4.08-3.97 (m, 2H), 3.68-3.51 (m, 6H), 3.49-3.40 (m, 1H), 2.71-2.61 (m, 2H), 2.59-2.52 (m, 4H), 2.37-2.26 (m, 2H), 2.00-1.91 (m, 2H), 1.89-1.75 (m, 2H), 1.72-1.61 (m, 2H), 1.42 (d, J=6.5 Hz, 3H).

### Example 109

¹H NMR (400 MHz, CDCl₃) δ 8.89 (d, J=2.0 Hz, 1H), 8.51 (d, J=9.1 Hz, 1H), 8.39 (s, 1H), 8.03 (dd, J=8.2, 2.3 Hz, 1H), 7.87 (dd, J=9.1, 1.6 Hz, 1H), 7.58 (d, J=8.2 Hz, 1H), 5.07-4.95 (m, 1H), 4.59 (q, J=6.5 Hz, 1H), 4.33-4.13 (m, 3H), 3.73 (s, 3H), 3.68-3.51 (m, 4H), 2.88-2.36 (m, 8H), 2.07-1.79 (m, 6H), 1.49 (d, J=6.6 Hz, 3H).

### Example 110

¹H NMR (400 MHz, CDCl₃) δ 8.88 (s, 1H), 8.51 (d, J=9.0 Hz, 1H), 8.38 (s, 1H), 8.02 (d, J=8.2 Hz, 1H), 7.86 (d, J=9.0 Hz, 1H), 7.58 (d, J=8.1 Hz, 1H), 5.09-4.95 (m, 1H), 4.64-4.53 (m, 1H), 4.24-4.11 (m, 2H), 3.73 (s, 3H), 3.68-3.49 (m, 4H), 3.29-3.19 (m, 4H), 2.87-2.66 (m, 6H), 2.56-2.29 (m, 2H), 2.03-1.84 (m, 6H), 1.49 (d, J=6.5 Hz, 3H).

### Example 111

¹H NMR (400 MHz, DMSO-d₆) δ 9.08 (d, J=2.1 Hz, 1H), 8.53 (s, 1H), 8.46 (d, J=9.1 Hz, 1H), 8.37-8.28 (m, 1H), 8.16 (d, J=9.1 Hz, 1H), 7.67 (d, J=8.2 Hz, 1H), 5.20-5.05 (m, 1H), 4.63-4.56 (m, 1H), 4.08-3.99 (m, 2H), 3.67-3.59 (m, 5H), 3.57-3.52 (m, 1H), 2.73-2.56 (m, 7H), 2.35-2.28 (m, 2H), 2.01-1.92 (m, 2H), 1.50 (t, J=7.1 Hz, 2H), 1.44 (d, J=6.5 Hz, 3H), 1.04 (d, J=2.1 Hz, 6H).

### Example 112

¹H NMR (400 MHz, CDCl₃) δ 8.94 (s, 1H), 8.56 (d, J=9.1 Hz, 1H), 8.43 (s, 1H), 8.08 (d, J=8.1 Hz, 1H), 7.92 (d, J=9.0 Hz, 1H), 7.65 (d, J=8.0 Hz, 1H), 5.14-4.99 (m, 1H), 4.71-4.59 (m, 1H), 4.32-4.20 (m, 2H), 4.00-3.90 (m, 1H), 3.79 (s, 3H), 3.73-3.57 (m, 4H), 3.29 (s, 3H), 3.07-2.71 (m, 8H), 2.13-2.04 (m, 1H), 2.00-1.84 (m, 3H), 1.56 (d, J=6.4 Hz, 3H).

### Example 113

¹H NMR (400 MHz, DMSO-d₆) δ 9.06 (s, 1H), 8.52 (s, 1H), 8.45 (d, J=9.1 Hz, 1H), 8.33 (d, J=9.0 Hz, 1H), 8.14 (d, J=9.1 Hz, 1H), 7.64 (d, J=8.2 Hz, 1H), 5.18-5.04 (m, 1H), 4.62-4.55 (m, 1H), 4.09-3.97 (m, 3H), 3.66-3.50 (m, 8H), 3.47-3.41 (m, 1H), 2.84-2.52 (m, 7H), 2.01-1.91 (m, 3H), 1.61-1.50 (m, 1H), 1.44 (d, J=6.5 Hz, 3H), 1.07-1.00 (m, 6H).

### Example 114

¹H NMR (400 MHz, DMSO-d₆) δ 9.07 (d, J=1.9 Hz, 1H), 8.53 (s, 1H), 8.46 (d, J=9.1 Hz, 1H), 8.35 (dd, J=8.2, 2.3 Hz, 1H), 8.15 (dd, J=9.1, 1.5 Hz, 1H), 7.66 (d, J=8.2 Hz, 1H), 5.20-5.05 (m, 1H), 4.60 (q, J=6.5 Hz, 1H), 4.11-3.99 (m, 2H), 3.67-3.60 (m, 5H), 3.57-3.54 (m, 2H), 2.89-2.80 (m, 1H), 2.74-2.52 (m, 6H), 2.39-2.26 (m, 2H), 2.02-1.92 (m, 2H), 1.90-1.78 (m, 2H), 1.75-1.63 (m, 2H), 1.44 (d, J=6.5 Hz, 3H).

### Example 116

### (R)-8-(6-(1-(2-(2-oxa-6-azaspiro[3.4]octan-6-yl)ethoxy)ethyl) pyridin-3-yl)-3-methyl-1-(tetrahydro-2H-pyran-4-yl)-1H-imidazo[4,5-c]cinnolin-2(3H)-one

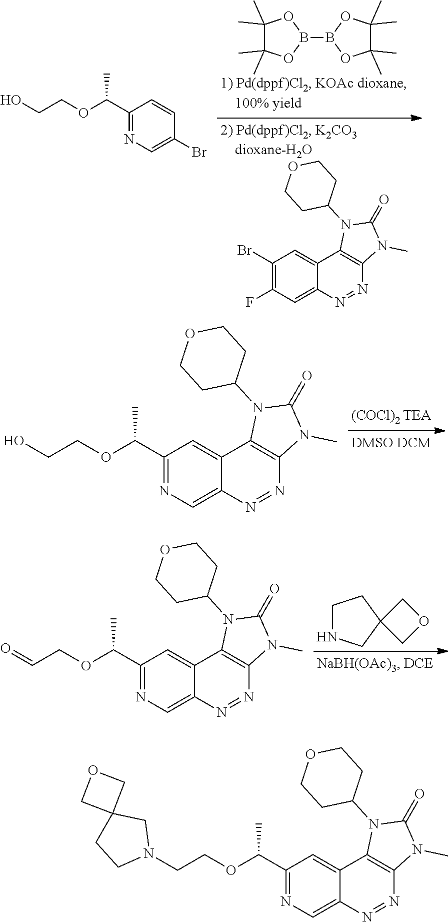

### Step 1: (R)-8-(6-(1-(2-hydroxyethoxy)ethyl)pyridin-3-yl)-3-methyl-1-(tetrahydro-2H-pyran-4-yl)-1H-imidazo[4,5-c]cinnolin-2(3H)-one

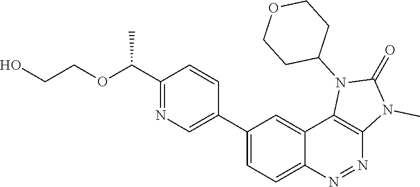 [SMILES: C[C@@H](OCCO)c1ccc(-c2ccc3nnc4c(c3c2)n(C2CCOCC2)c(=O)n4C)cn1]

The crude product was prepared in a similar fashion to Example 1, which was purified by chromatography on silica gel (DCM:MeOH=100:1 to 10:1) to give the desired product (1.20 g, 65.6% yield for two steps) as a brown solid. LC-MS (ESI) m/z: 450 [M+H]⁺.

### Step 2: (R)-2-(1-(5-(3-methyl-2-oxo-1-(tetrahydro-2H-pyran-4-yl)-2,3-dihydro-1H-imidazo[4,5-c]cinnolin-8-yl)pyridin-2-yl)ethoxy)acetaldehyde

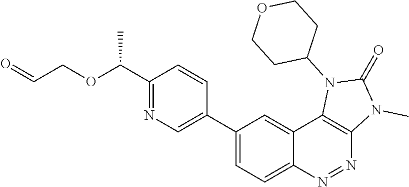 [SMILES: C[C@@H](OCC=O)c1ccc(-c2ccc3nnc4c(c3c2)n(C2CCOCC2)c(=O)n4C)cn1]

To a solution of oxalyl chloride (0.343 mL, 4.05 mmol) in dry DCM (5 mL) was added dropwise DMSO (0.574 mL, 8.10 mmol) at −78° C. under N₂ and the resulting reaction mixture was stirred at −78° C. for 15 min. Then to the reaction mixture was added dropwise a solution of (R)-8-(6-(1-(2-hydroxyethoxy)ethyl)pyridin-3-yl)-3-methyl-1-(tetrahydro-2H-pyran-4-yl)-1H-imidazo[4,5-c]cinnolin-2(3H)-one (1.20 g, 2.70 mmol) in dry DCM (10 mL) at −78° C. After stirring for 20 min at −78° C., TEA (1.87 mL, 13.5 mmol) was added. The resulting mixture was then slowly warmed to room temperature. The mixture was quenched with water (30 mL) and extracted with EtOAc (20 mL×2). The combined organic layers were washed with brine (40 mL), dried over anhydrous Na₂SO₄ and filtered, the filtrate was concentrated under reduced pressure to give the crude desired product (1.10 g) as a brown solid. LC-MS (ESI) m z: 448 [M+H]⁺.

### Step 3: (R)-8-(6-(1-(2-(2-oxa-6-azaspiro[3.4]octan-6-yl)ethoxy)ethyl)pyridin-3-yl)-3-methyl-1-(tetrahydro-2H-pyran-4-yl)-1H-imidazo[4,5-c]cinnolin-2(3H)-one

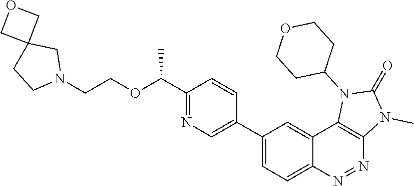 [SMILES: C[C@@H](OCCN1CCC2(COC2)C1)c1ccc(-c2ccc3nnc4c(c3c2)n(C2CCOCC2)c(=O)n4C)cn1]

To a mixture of 2-oxa-6-azaspiro[3.4]octane (45.0 mg, 0.394 mmol) and (R)-2-(1-(5-(3-methyl-2-oxo-1-(tetrahydro-2H-pyran-4-yl)-2,3-dihydro-1H-imidazo[4,5-c]cinnolin-8-yl)pyridin-2-yl)ethoxy)acetaldehyde (176 mg, 0.394 mmol) in DCE (4 mL) was added slowly sodium triacetoxyborohydride (251 mg, 1.18 mmol) at 0° C., and then the reaction mixture was stirred at room temperature overnight. The mixture was poured into ice sat. NaHCO₃ solution (20 mL) and extracted with DCM (15 mL×2). The organic layers were washed with brine (30 mL), dried over anhydrous Na₂SO₄ and filtered, the filtrate was concentrated under reduced pressure. The residue was purified by flash chromatography (DCM:MeOH=9:1) to give the desired product (40.0 mg, 18.7% yield) as a brown solid. ¹H NMR (400 MHz, CDCl₃) δ 8.96 (d, J=2.0 Hz, 1H), 8.58 (d, J=9.0 Hz, 1H), 8.47 (s, 1H), 8.10 (dd, J=8.1, 2.3 Hz, 1H), 7.94 (dd, J=9.0, 1.5 Hz, 1H), 7.63 (d, J=8.2 Hz, 1H), 5.16-4.95 (m, 1H), 4.75-4.57 (m, 5H), 4.31-4.16 (m, 2H), 3.80 (s, 3H), 3.75-3.60 (m, 4H), 3.23-2.74 (m, 8H), 2.29-2.16 (m, 2H), 2.02-1.90 (m, 2H), 1.58 (d, J=6.5 Hz, 3H). LC-MS (ESI) m z: 545 [M+H]⁺.

The following compounds were prepared according to the above described methods using different starting materials.

| | | | MS |
|---|---|---|---|
| Ex# | Structure | Name | m/z |
| 117 | 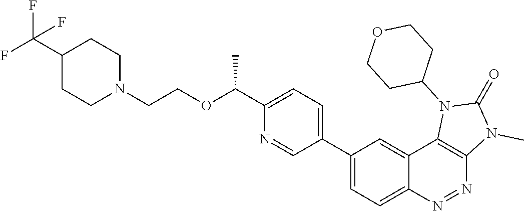 [SMILES: C[C@@H](OCCN1CCC(C(F)(F)F)CC1)c1ccc(-c2ccc3nnc4c(c3c2)n(C2CCOCC2)c(=O)n4C)cn1] | (R)-3-methyl-1-(tetrahydro- 2H-pyran-4-yl)-8-(6-(1-(2-(4- (trifluoromethyl)piperidin-1- yl)ethoxy)ethyl)pyridin-3-yl)- 1H-imidazo[4,5-c]cinnolin- 2(3H)-one | 585 [M + H]⁺ |
| 118 | 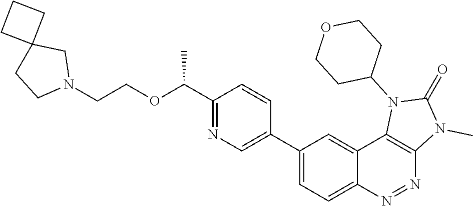 [SMILES: C[C@@H](OCCN1CCC2(CCC2)C1)c1ccc(-c2ccc3nnc4c(c3c2)n(C2CCOCC2)c(=O)n4C)cn1] | (R)-8-(6-(1-(2-(6- azaspiro[3.4]octan-6- yl)ethoxy)ethyl)pyridin-3-yl)- 3-methyl-1-(tetrahydro-2H- pyran-4-yl)-1H-imidazo[4,5- c]cinnolin-2(3H)-one | 543 [M + H]⁺ |
| 119 | 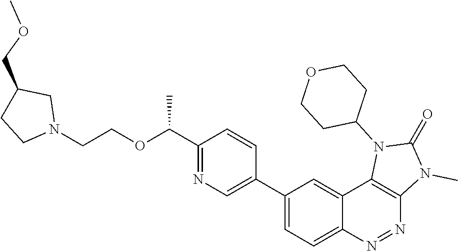 [SMILES: COC[C@@H]1CCN(CCO[C@H](C)c2ccc(-c3ccc4nnc5c(c4c3)n(C3CCOCC3)c(=O)n5C)cn2)C1] | 8-(6-((R)-1-(2-((R)-3- (methoxymethyl)pyrrolidin-1- yl)ethoxy)ethyl)pyridin-3-yl)- 3-methyl-1-(tetrahydro-2H- pyran-4-yl)-1H-imidazo[4,5- c]cinnolin-2(3H)-one | 547 [M + H]⁺ |
| 120 | 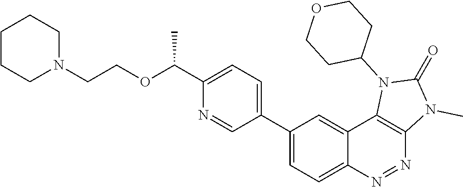 [SMILES: C[C@@H](OCCN1CCCCC1)c1ccc(-c2ccc3nnc4c(c3c2)n(C2CCOCC2)c(=O)n4C)cn1] | (R)-3-methyl-8-(6-(1-(2- (piperidin-1- yl)ethoxy)ethyl)pyridin-3-yl)- 1-(tetrahydro-2H-pyran-4-yl)- 1H-imidazo[4,5-c]cinnolin- 2(3H)-one | 517 [M + H]⁺ |
| 121 | 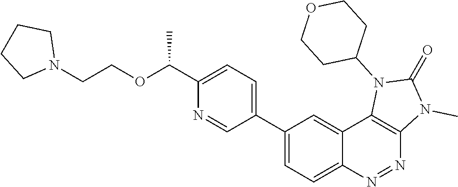 [SMILES: C[C@@H](OCCN1CCCC1)c1ccc(-c2ccc3nnc4c(c3c2)n(C2CCOCC2)c(=O)n4C)cn1] | (R)-3-methyl-8-(6-(1-(2- (pyrrolidin-1- yl)ethoxy)ethyl)pyridin-3-yl)- 1-(tetrahydro-2H-pyran-4-yl)- 1H-imidazo[4,5-c]cinnolin- 2(3H)-one | 503 [M + H]⁺ |
| 122 | 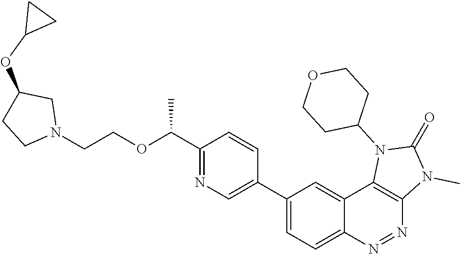 [SMILES: C[C@@H](OCCN1CC[C@@H](OC2CC2)C1)c1ccc(-c2ccc3nnc4c(c3c2)n(C2CCOCC2)c(=O)n4C)cn1] | 8-(6-((R)-1-(2-((R)-3- cyclopropoxypyrrolidin-1- yl)ethoxy)ethyl)pyridin-3-yl)- 3-methyl-1-(tetrahydro-2H- pyran-4-yl)-1H-imidazo[4,5- c]cinnolin-2(3H)-one | 559 [M + H]⁺ |
| 123 | 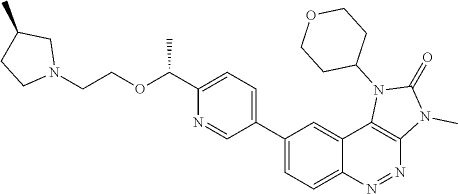 [SMILES: C[C@@H]1CCN(CCO[C@H](C)c2ccc(-c3ccc4nnc5c(c4c3)n(C3CCOCC3)c(=O)n5C)cn2)C1] | 3-methyl-8-(6-((R)-1-(2-((R)- 3-methylpyrrolidin-1- y)ethoxy)ethyl)pyridin-3-yl)- 1-(tetrahydro-2H-pyran-4-yl)- 1H-imidazo[4,5-c]cinnolin- 2(3H)-one | 517 [M + H]⁺ |
| 124 | 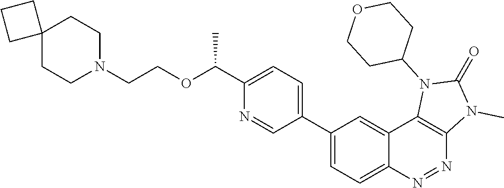 [SMILES: C[C@@H](OCCN1CCC2(CCC2)CC1)c1ccc(-c2ccc3nnc4c(c3c2)n(C2CCOCC2)c(=O)n4C)cn1] | (R)-8-(6-(1-(2-(7- azaspiro[3.5]nonan-7- yl)ethoxy)ethyl)pyridin-3-yl)- 3-methyl-1-(tetrahydro-2H- pyran-4-yl)-1H-imidazo[4,5- c]cinnolin-2(3H)-one | 557 [M + H]⁺ |
| 125 | 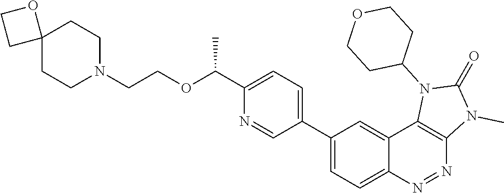 [SMILES: C[C@@H](OCCN1CCC2(CCO2)CC1)c1ccc(-c2ccc3nnc4c(c3c2)n(C2CCOCC2)c(=O)n4C)cn1] | (R)-8-(6-(1-(2-(1-oxa-7- azaspiro[3.5]nonan-7- yl)ethoxy)ethyl)pyridin-3-yl)- 3-methyl-1-(tetrahydro-2H- pyran-4-yl)-1H-imidazo[4,5- c]cinnolin-2(3H)-one | 559 [M + H]⁺ |
| 126 | 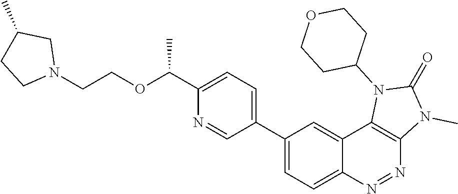 [SMILES: C[C@H]1CCN(CCO[C@H](C)c2ccc(-c3ccc4nnc5c(c4c3)n(C3CCOCC3)c(=O)n5C)cn2)C1] | 3-methyl-8-(6-((R)-1-(2-((S)-3- methylpyrrolidin-1- yl)ethoxy)ethyl)pyridin-3-yl)- 1-(tetrahydro-2H-pyran-4-yl)- 1H-imidazo[4,5-c]cinnolin- 2(3H)-one | 517 [M + H]⁺ |
| 127 | 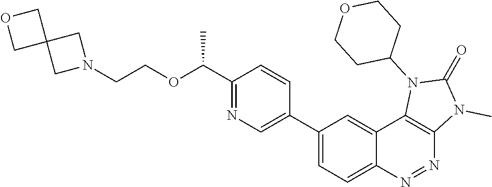 [SMILES: C[C@@H](OCCN1CC2(COC2)C1)c1ccc(-c2ccc3nnc4c(c3c2)n(C2CCOCC2)c(=O)n4C)cn1] | (R)-8-(6-(1-(2-(2-oxa-6- azaspiro[3.3]heptan-6- yl)ethoxy)ethyl)pyridin-3-yl)- 3-methyl-1-(tetrahydro-2H- pyran-4-yl)-1H-imidazo[4,5- c]cinnolin-2(3H)-one | 531 [M + H]⁺ |
| 128 | 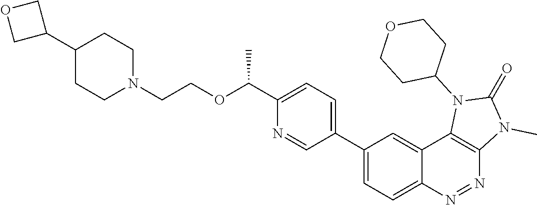 [SMILES: C[C@@H](OCCN1CCC(C2COC2)CC1)c1ccc(-c2ccc3nnc4c(c3c2)n(C2CCOCC2)c(=O)n4C)cn1] | (R)-3-methyl-8-(6-(1-(2-(4- (oxetan-3-yl)piperidin-1- yl)ethoxy)ethyl)pyridin-3-yl)- 1-(tetrahydro-2H-pyran-4-yl)- 1H-imidazo[4,5-c]cinnolin- 2(3H)-one | 573 [M + H]⁺ |
| 129 | 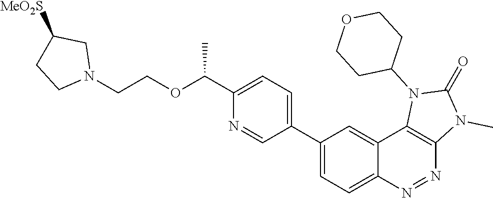 [SMILES: C[C@@H](OCCN1CC[C@@H](S(C)(=O)=O)C1)c1ccc(-c2ccc3nnc4c(c3c2)n(C2CCOCC2)c(=O)n4C)cn1] | 3-methyl-8-(6-((R)-1-(2-((R)- 3-(methylsulfonyl)pyrrolidin- 1-yl)ethoxy)ethyl)pyridin-3- yl)-1-(tetrahydro-2H-pyran-4- yl)-1H-imidazo[4,5-c]cinnolin- 2(3H)-one | 581 [M + H]⁺ |
| 130 | 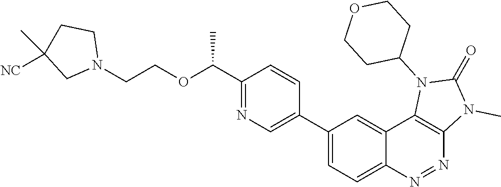 [SMILES: C[C@@H](OCCN1CCC(C)(C#N)C1)c1ccc(-c2ccc3nnc4c(c3c2)n(C2CCOCC2)c(=O)n4C)cn1] | 3-methyl-1-(2-((R)-1-(5-(3- methyl-2-oxo-1-(tetrahydro- 2H-pyran-4-yl)-2,3-dihydro- 1H-imidazo[4.5-c]cinnolin-8- yl)pyridin-2-yl)ethoxy)ethyl) pyrrolidine-3-carbonitrile | 542 [M + H]⁺ |

### Example 117

¹H NMR (400 MHz, DMSO-d₆) δ 9.07 (d, J=2.2 Hz, 1H), 8.53 (s, 1H), 8.46 (d, J=9.1 Hz, 1H), 8.35 (dd, J=8.2, 2.4 Hz, 1H), 8.15 (dd, J=9.1, 1.5 Hz, 1H), 7.65 (d, J=8.2 Hz, 1H), 5.18-5.07 (m, 1H), 4.60 (q, J=6.4 Hz, 1H), 4.11-3.99 (m, 2H), 3.67-3.59 (m, 5H), 3.58-3.53 (m, 1H), 3.50-3.45 (m, 1H), 2.99-2.91 (m, 2H), 2.74-2.53 (m, 4H), 2.29-2.17 (m, 1H), 2.06-2.00 (m, 2H), 1.98-1.91 (m, 2H), 1.79-1.71 (m, 2H), 1.50-1.37 (m, 5H).

### Example 118

¹H NMR (400 MHz, CDCl₃) δ 8.97 (s, 1H), 8.58 (d, J=9.2 Hz, 1H), 8.46 (s, 1H), 8.11 (d, J=8.2 Hz, 1H), 7.93 (d, J=9.2 Hz, 1H), 7.61 (d, J=8.1 Hz, 1H), 5.14-5.02 (m, 1H), 4.77-4.66 (m, 1H), 4.34-4.21 (m, 2H), 3.96-3.75 (m, 6H), 3.69-3.55 (m, 3H), 3.21-3.09 (m, 2H), 2.93-2.77 (m, 2H), 2.27-1.87 (m, 12H), 1.58 (d, J=6.6 Hz, 3H).

### Example 119

¹H NMR (400 MHz, DMSO-d₆) δ 9.07 (d, J=2.0 Hz, 1H), 8.53 (s, 1H), 8.46 (d, J=9.1 Hz, 1H), 8.35 (dd, J=8.2, 2.4 Hz, 1H), 8.15 (dd, J=9.1, 1.5 Hz, 1H), 7.65 (d, J=8.2 Hz, 1H), 5.19-5.06 (m, 1H), 4.61 (q, J=6.5 Hz, 1H), 4.12-4.01 (m, 2H), 3.68-3.51 (m, 7H), 3.48-3.42 (m, 1H), 3.23-3.18 (m, 5H), 2.74-2.56 (m, 6H), 2.36-2.24 (m, 2H), 1.99-1.92 (m, 2H), 1.87-1.76 (m, 1H), 1.44 (d, J=6.5 Hz, 3H), 1.38-1.30 (m, 1H).

### Example 120

¹H NMR (400 MHz, CDCl₃) δ 8.96 (d, J=1.8 Hz, 1H), 8.57 (d, J=9.0 Hz, 1H), 8.46 (s, 1H), 8.09 (dd, J=8.1, 2.1 Hz, 1H), 7.93 (d, J=9.1 Hz, 1H), 7.61 (d, J=8.1 Hz, 1H), 5.17-5.01 (m, 1H), 4.67 (q, J=6.5 Hz, 1H), 4.29-4.20 (m, 2H), 3.90-3.73 (m, 5H), 3.65 (t, J=11.4 Hz, 2H), 3.00-2.74 (m, 8H), 2.02-1.93 (m, 2H), 1.90-1.79 (m, 4H), 1.63-1.50 (m, 5H).

### Example 121

¹H NMR (400 MHz, CDCl₃) δ 8.98 (d, J=2.1 Hz, 1H), 8.57 (d, J=9.1 Hz, 1H), 8.47 (s, 1H), 8.11 (dd, J=8.2, 2.3 Hz, 1H), 7.93 (dd, J=9.1, 1.6 Hz, 1H), 7.61 (d, J=8.2 Hz, 1H), 5.16-5.02 (m, 1H), 4.72 (q, J=6.5 Hz, 1H), 4.34-4.20 (m, 2H), 4.03-3.88 (m, 2H), 3.79 (s, 3H), 3.72-3.63 (m, 2H), 3.49-3.36 (m, 4H), 3.30 (t, J=4.8 Hz, 2H), 2.93-2.79 (m, 2H), 2.19-2.11 (m, 4H), 2.01-1.91 (m, 2H), 1.59 (d, J=6.5 Hz, 3H).

### Example 122

¹H NMR (400 MHz, CDCl₃) δ 8.96 (d, J=1.9 Hz, 1H), 8.57 (d, J=9.0 Hz, 1H), 8.47 (s, 1H), 8.12 (dd, J=8.1, 2.3 Hz, 1H), 7.93 (dd, J=9.0, 1.5 Hz, 1H), 7.64 (d, J=8.1 Hz, 1H), 5.11 (s, 1H), 4.77-4.63 (m, 1H), 4.36-4.19 (m, 3H), 3.90-3.82 (m, 2H), 3.80 (s, 3H), 3.72-3.60 (m, 4H), 3.34-3.09 (m, 5H), 2.93-2.80 (m, 2H), 2.28-2.14 (m, 2H), 2.03-1.91 (m, 3H), 1.59 (d, J=6.5 Hz, 3H), 0.68-0.47 (m, 4H).

### Example 123

¹H NMR (600 MHz, DMSO-d₆) δ 9.12 (d, J=2.0 Hz, 1H), 8.54 (s, 1H), 8.48 (d, J=9.0 Hz, 1H), 8.39 (dd, J=8.2, 2.4 Hz, 1H), 8.17 (dd, J=9.1, 1.6 Hz, 1H), 7.71 (d, J=8.2 Hz, 1H), 5.19-5.07 (m, 1H), 4.72 (q, J=6.5 Hz, 1H), 4.11-4.00 (m, 2H), 3.66-3.63 (m, 2H), 3.62 (s, 3H), 3.61-3.59 (m, 2H), 3.44-3.06 (m, 5H), 2.72-2.60 (m, 3H), 2.37-2.29 (m, 1H), 2.22-2.08 (m, 1H), 2.01-1.93 (m, 2H), 1.66-1.48 (m, 4H), 1.08 (d, J=6.7 Hz, 3H).

### Example 124

¹H NMR (400 MHz, DMSO-d₆) δ 9.07 (d, J=2.1 Hz, 1H), 8.53 (s, 1H), 8.46 (d, J=9.1 Hz, 1H), 8.35 (dd, J=8.2, 2.4 Hz, 1H), 8.15 (dd, J=9.1, 1.6 Hz, 1H), 7.66 (d, J=8.2 Hz, 1H), 5.19-5.07 (m, 1H), 4.59 (q, J=6.5 Hz, 1H), 4.09-4.00 (m, 2H), 3.67-3.59 (m, 5H), 3.57-3.52 (m, 1H), 3.49-3.43 (m, 1H), 2.74-2.59 (m, 2H), 2.57-2.52 (m, 1H), 2.48-2.43 (m, 1H), 2.38-2.23 (m, 4H), 2.01-1.93 (m, 2H), 1.85-1.77 (m, 2H), 1.72-1.64 (m, 4H), 1.52 (t, J=5.4 Hz, 4H), 1.43 (d, J=6.5 Hz, 3H).

### Example 125

¹H NMR (400 MHz, CDCl₃) δ 8.94 (d, J=2.0 Hz, 1H), 8.57 (d, J=9.1 Hz, 1H), 8.45 (s, 1H), 8.08 (dd, J=8.2, 2.3 Hz, 1H), 7.93 (dd, J=9.1, 1.6 Hz, 1H), 7.63 (d, J=8.1 Hz, 1H), 5.19-4.99 (m, 1H), 4.66 (q, J=6.6 Hz, 1H), 4.52 (t, J=7.8 Hz, 2H), 4.30-4.19 (m, 2H), 3.80 (s, 3H), 3.75-3.59 (m, 4H), 2.94-2.61 (m, 8H), 2.40 (t, J=7.8 Hz, 2H), 2.11-1.93 (m, 6H), 1.55 (d, J=6.6 Hz, 3H).

### Example 126

¹H NMR (400 MHz, DMSO-d₆) δ 9.12 (d, J=1.9 Hz, 1H), 8.54 (s, 1H), 8.48 (d, J=9.1 Hz, 1H), 8.39 (dd, J=8.2, 2.4 Hz, 1H), 8.16 (dd, J=9.1, 1.6 Hz, 1H), 7.70 (d, J=8.2 Hz, 1H), 5.20-5.08 (m, 1H), 4.72 (q, J=6.5 Hz, 1H), 4.10-4.00 (m, 2H), 3.81-3.70 (m, 1H), 3.67-3.60 (m, 6H), 3.16-3.06 (m, 2H), 2.73-2.59 (m, 4H), 2.39-2.27 (m, 2H), 2.23-2.07 (m, 2H), 2.01-1.93 (m, 2H), 1.68-1.58 (m, 1H), 1.52 (d, J=6.5 Hz, 3H), 1.07 (d, J=6.7 Hz, 3H).

### Example 127

¹H NMR (400 MHz, CDCl₃) δ 8.97 (s, 1H), 8.57 (d, J=9.0 Hz, 1H), 8.48 (s, 1H), 8.11 (d, J=6.0 Hz, 1H), 7.93 (d, J=9.3 Hz, 1H), 7.59 (d, J=8.1 Hz, 1H), 5.16-5.02 (m, 1H), 4.81 (s, 3H), 4.71-4.55 (m, 1H), 4.31-4.20 (m, 2H), 4.12-3.94 (m, 2H), 3.82-3.74 (m, 4H), 3.71-3.52 (m, 6H), 3.05-2.79 (m, 4H), 2.04-1.91 (m, 2H), 1.56 (d, J=6.6 Hz, 3H).

### Example 128

¹H NMR (400 MHz, CDCl₃) δ 8.95 (s, 1H), 8.57 (d, J=9.0 Hz, 1H), 8.46 (s, 1H), 8.09 (d, J=6.3 Hz, 1H), 7.93 (d, J=9.3 Hz, 1H), 7.60 (d, J=8.3 Hz, 1H), 5.16-5.02 (m, 1H), 4.76 (t, J=6.9 Hz, 2H), 4.67 (q, J=6.5 Hz, 1H), 4.44 (t, J=6.1 Hz, 2H), 4.26 (dd, J=11.5, 4.4 Hz, 2H), 3.85-3.71 (m, 5H), 3.64 (t, J=11.5 Hz, 2H), 3.43-3.22 (m, 2H), 3.00-2.74 (m, 5H), 2.51-2.34 (m, 2H), 1.97 (d, J=9.5 Hz, 3H), 1.84-1.65 (m, 4H), 1.56 (d, J=6.5 Hz, 3H).

### Example 129

¹H NMR (600 MHz, DMSO-d₆) δ 9.11 (d, J=2.2 Hz, 1H), 8.54 (s, 1H), 8.47 (d, J=9.1 Hz, 1H), 8.38 (dd, J=8.2, 2.4 Hz, 1H), 8.18 (dd, J=9.1, 1.6 Hz, 1H), 7.71 (d, J=8.2 Hz, 1H), 5.18-5.09 (m, 1H), 4.74 (q, J=6.5 Hz, 1H), 4.25-4.15 (m, 1H), 4.10-4.02 (m, 2H), 3.82-3.75 (m, 2H), 3.69-3.66 (m, 4H), 3.61 (s, 3H), 3.50-3.48 (m, 4H), 3.13 (s, 3H), 2.69-2.60 (m, 2H), 2.44-2.35 (m, 2H), 2.00-1.93 (m, 2H), 1.51 (d, J=6.5 Hz, 3H).

### Example 130

¹H NMR (400 MHz, DMSO-d₆) δ 9.07 (s, 1H), 8.55-8.40 (m, 2H), 8.33 (d, J=4.4 Hz, 1H), 8.12 (d, J=9.1 Hz, 1H), 7.69 (s, 1H), 5.20-5.01 (m, 1H), 4.68-4.52 (m, 1H), 4.14-3.97 (m, 2H), 3.70-3.53 (m, 7H), 3.12-2.52 (m, 8H), 2.44-2.31 (m, 1H), 2.26-2.10 (m, 1H), 2.03-1.90 (m, 2H), 1.89-1.68 (m, 1H), 1.52-1.34 (m, 5H).

### Example 131

### 8-(6-((2-(5-azaspiro[2.4]heptan-5-yl)ethoxy)methyl)pyridin-3-yl)-7-fluoro-1-isopropyl-3-methyl-1H-imidazo[4,5-c]cinnolin-2(3H)-one

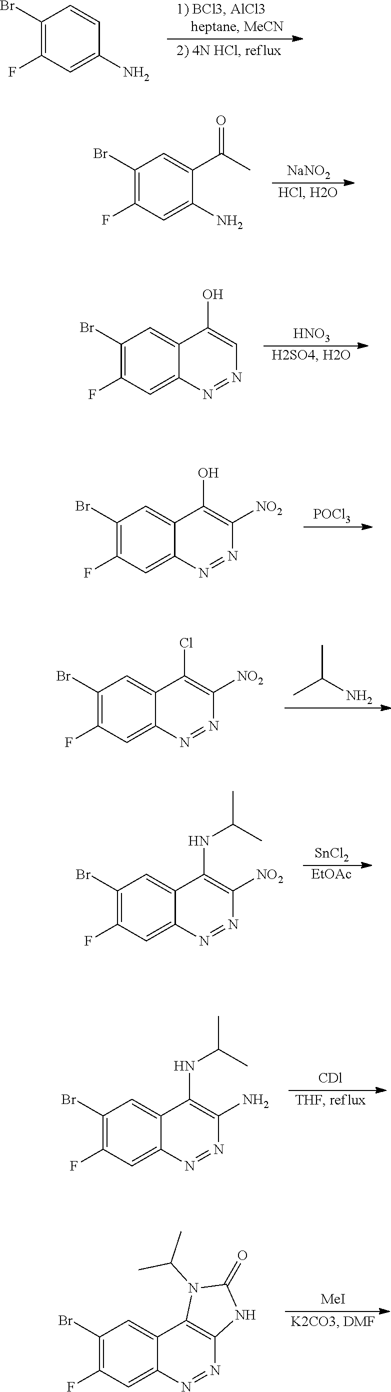

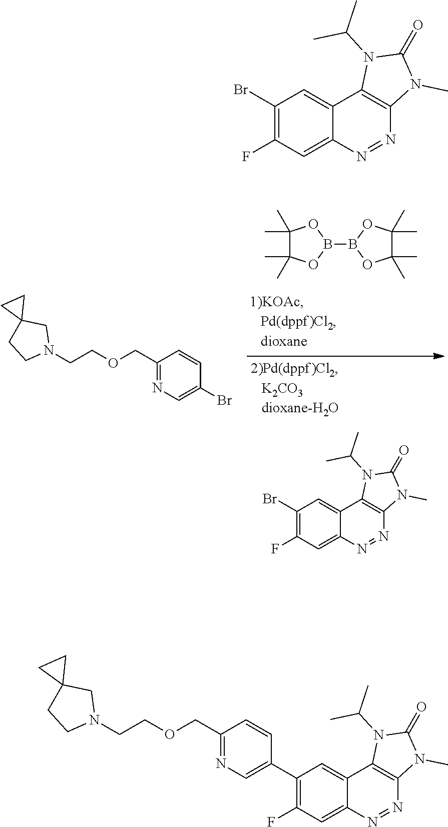

### Step 1: 1-(2-amino-5-bromo-4-fluorophenyl)ethanone

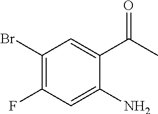

To a solution of BCl₃ (1.5 L, 1 M in DCM, 1.5 mol) stirred at 0° C., was added 4-bromo-3-fluoroaniline (250 g, 1.32 mol) in DCE (2.5 L) dropwise. MeCN (210 mL) and AlCl₃ (210 g, 1.58 mol) were added to the solution in portions. The resulting mixture was stirred at 120° C. for 16 hrs. After the reaction was cooled down, 2 N HCl aqueous solution (3.1 L) was added dropwise to the mixture at 0° C., the resulting mixture was stirred at 100° C. for 2 h. The reaction mixture was poured into ice water and extracted with DCM (2 L×2). The combined organic phases were washed with brine, dried over anhydrous Na₂SO₄, filtered and concentrated. The residue was purified by column chromatography on silica gel (EA:PE=1:5) to give the desired product 1-(2-amino-5-bromo-4-fluorophenyl)ethenone (60 g, 20% yield) as a white solid. MS: m/z 232 [M+H]⁺. ¹H NMR (400 MHz, CDCl₃) δ 7.88 (d, J=7.8 Hz, 1H), 6.52-6.39 (m, 3H), 2.54 (s, 6H).

### Step 2: 6-bromo-7-fluorocinnolin-4-ol

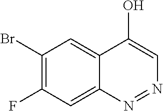 [SMILES: Oc1cnnc2cc(F)c(Br)cc12]

To a stirred solution of 1-(2-amino-5-bromo-4-fluorophenyl)ethan-1-one (120 g, 519.5 mmol) in conc. HCl (1.7 L) and H₂O (380 mL) was added dropwise a solution of NaNO₂ (40 g, 580 mmol) in H₂O (100 mL) at 0° C. After stirred at 0° for 1 h, the resulting mixture was heated to 65° C. for 16 hrs. The reaction mixture was poured into ice water and the precipitate was collected by filtration. The solid was washed with water and dried under vacuum to give the desired product (100 g, 80% yield) as a yellow solid. ¹H NMR (400 MHz, DMSO-d₆) δ 13.62 (s, 1H), 8.25 (d, J=7.4 Hz, 1H), 7.79 (s, 1H), 7.46 (d, J=9.3 Hz, 1H). LC-MS: m/z 243 [M+H]⁺.

### Step 3:6-bromo-7-fluoro-3-nitrocinnolin-4-ol

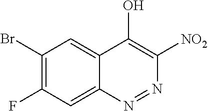 [SMILES: O=[N+]([O-])c1nnc2cc(F)c(Br)cc2c1O]

To a solution of 6-bromo-7-fluorocinnolin-4-ol (200 g, 826.4 mmol) in fuming HNO₃ (600 mL) stirred at 0° C., was added conc. H₂SO₄ (200 mL) dropwise carefully in 10 min. The resulting mixture was stirred at 60° C. for 3 h. After cooled to room temperature, the reaction mixture was poured into ice water. The precipitate was collected by filtration, washed with water, filtered and dried under vacuum to give the desired product (180 g, 76% yield) as a yellow solid. MS: m/z 288 [M+H]⁺. ¹H NMR (400 MHz, DMSO-d₆) δ 14.46 (s, 1H), 8.46 (d, J=7.1 Hz, 1H), 7.68 (d, J=8.8 Hz, 1H).

### Step 4: 6-bromo-4-chloro-7-fluoro-3-nitrocinnoline

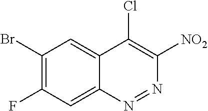 [SMILES: O=[N+]([O-])c1nnc2cc(F)c(Br)cc2c1Cl]

To a solution of 6-bromo-7-fluoro-3-nitrocinnolin-4-ol (180 g, 627 mmol) in DMF (900 mL) stirred at 0° C., was added POCl₃ (125 g, 815 mmol) dropwise. The resulting mixture was stirred at room temperature for 16 h. The reaction mixture was poured into ice water, the solid was filtered, washed with water, filtered and dried to give the crude product 6-bromo-4-chloro-7-fluoro-3-nitrocinnoline (145 g, 76% yield) as a yellow solid, which was used for next step without further purification. ¹H NMR (400 MHz, CDCl₃) δ 8.70 (d, J=6.6 Hz, 1H), 8.38 (d, J=7.7 Hz, 1H).

### Step 5: 6-bromo-7-fluoro-N-isopropyl-3-nitrocinnolin-4-amine

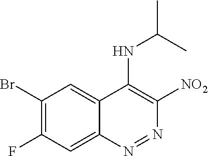 [SMILES: CC(C)Nc1c([N+](=O)[O-])nnc2cc(F)c(Br)cc12]

To a solution of 6-bromo-4-chloro-7-fluoro-3-nitrocinnoline (145 g, 475 mmol) and Et₃N (96 g, 951 mmol) in DCM (1.5 L) stirred at room temperature, was added isopropylamine (42 g, 715 mmol). The resulting mixture was stirred at room temperature for 2 h. The crude mixture was wash with water (1 L) and concentrated to dryness, the residue was purified by column chromatography on silica gel (EA:PE=1:1) to give the desired product (102 g, 65% yield) as a yellow solid. MS: m/z 329 [M+H]⁺. ¹H NMR (400 MHz, CDCl₃) δ 9.10 (s, 1H), 8.54 (d, J=6.5 Hz, 1H), 8.14 (d, J=8.2 Hz, 1H), 4.46 (dp, J=8.3, 6.2 Hz, 1H), 1.55 (d, J=6.3 Hz, 6H).

### Step 6: 6-bromo-7-fluoro-N4-isopropylcinnoline-3,4-diamine

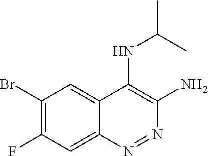 [SMILES: CC(C)Nc1c(N)nnc2cc(F)c(Br)cc12]

To a solution of 6-bromo-7-fluoro-N-isopropyl-3-nitrocinnolin-4-amine (102 g, 311 mmol) in EtOAc (1 L) stirred at room temperature, was added SnCl₂·2H₂O (281 g, 1244 mmol). The resulting mixture was stirred at 80° C. for 2 h. The crude mixture was basified with aq. NaHCO₃ to adjust pH=9 and filtered. The filtrate was extracted with EtOAc (1 L). The organic phase was washed with water and brine, filtered and the filtrate was concentrated to give the desired product (85 g, 88% yield) as a yellow solid. MS: m/z 299 [M+H]⁺. ¹H NMR (400 MHz, DMSO-d₆) δ 8.43 (d, J=7.2 Hz, 1H), 7.80 (d, J=9.7 Hz, 1H), 5.95 (s, 2H), 5.34 (d, J=9.8 Hz, 1H), 4.05-3.90 (m, 1H), 1.16 (d, J=6.3 Hz, 6H).

### Step 7: 8-bromo-7-fluoro-1-isopropyl-1H-imidazo[4,5-c]cinnolin-2(3H)-one

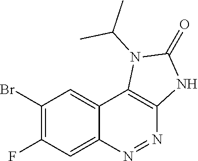 [SMILES: CC(C)n1c(=O)[nH]c2nnc3cc(F)c(Br)cc3c21]

To a solution of 6-bromo-7-fluoro-N4-isopropylcinnoline-3,4-diamine (85 g, 285 mmol) in THE (1 L) stirred at room temperature, was added CDI (184 g, 1140 mmol). The resulting mixture was stirred at 70° C. for 16 h. The reaction mixture was concentrated, the residue was poured into ice water, the solid was filtered, washed with water, dried to give the desired product (81 g, 88% yield) as a yellow solid. MS: m/z 325 [M+H]⁺. ¹H NMR (400 MHz, CDCl₃) δ 8.38 (d, J=6.7 Hz, 1H), 8.11 (d, J=8.9 Hz, 1H), 5.06 (s, 1H), 3.74 (s, 3H), 1.77 (d, J=6.9 Hz, 6H).

### Step 8: 8-bromo-7-fluoro-1-isopropyl-3-methyl-1H-imidazo[4,5-c]cinnolin-2(3H)-one

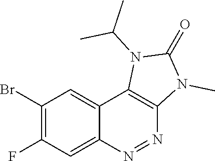 [SMILES: CC(C)n1c(=O)n(C)c2nnc3cc(F)c(Br)cc3c21]

To a solution of 8-bromo-7-fluoro-1-isopropyl-1H-imidazo[4,5-c]cinnolin-2(3H)-one (55 g, 170 mmol) in DMF (500 mL) was added K₂CO₃ (70 g, 510 mmol) at room temperature, followed by Mel (60 g, 425 mmol). After stirred at room temperature for 2 h, the reaction mixture was poured into ice water and the precipitate was collected by filtration. The residue was purified by chromatography on silica gel (DCM:MeOH=30:1) to give the desired product (48 g, 84% yield) as a yellow solid.

¹H NMR (400 MHz, DMSO-d₆) δ 12.49 (s, 1H), 8.66 (d, J=6.9 Hz, 1H), 8.17 (d, J=9.6 Hz, 1H), 5.10 (p, J=6.8 Hz, 1H), 1.59 (d, J=6.7 Hz, 6H). LC-MS: m/z 339 [M+H]⁺.

### Step 9: 8-(6-((2-(5-azaspiro[2.4]heptan-5-yl)ethoxy)methyl)pyridin-3-yl)-7-fluoro-1-isopropyl-3-methyl-1H-imidazo[4,5-c]cinnolin-2(3H)-one

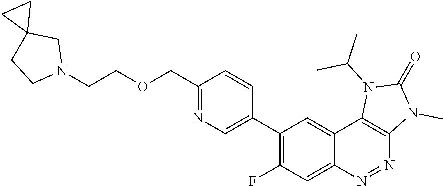 [SMILES: CC(C)n1c(=O)n(C)c2nnc3cc(F)c(-c4ccc(COCCN5CCC6(CC6)C5)nc4)cc3c21]

The crude product was prepared in a similar fashion to Example 1, which was purified by flash chromatography (DCM:MeOH=9:1) to give the desired product (30.0 mg, 19.0% yield) as a yellow solid. ¹H NMR (400 MHz, DMSO-d₆) δ 8.87 (s, 1H), 8.42 (d, J=7.7 Hz, 1H), 8.28 (s, 1H), 8.24 (d, J=11.5 Hz, 1H), 8.21-8.16 (m, 1H), 7.65 (d, J=8.1 Hz, 1H), 5.30-5.21 (m, 1H), 4.67 (s, 2H), 3.68 (t, J=5.9 Hz, 2H), 3.59 (s, 3H), 2.77-2.68 (m, 4H), 2.52 (s, 2H), 1.73 (t, J=6.9 Hz, 2H), 1.63 (d, J=6.7 Hz, 6H), 0.57-0.46 (in, 4H). LC-MS (ESI) m/z: 491 [M+H]^{m}.

The following compounds were prepared according to the above described methods using different starting materials.

| Ex# | Structure | Name | MS m/z |
|---|---|---|---|
| 132 | 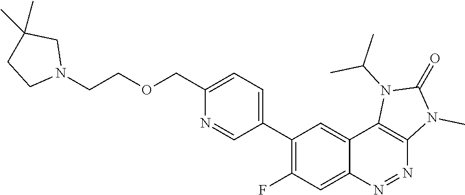 | 8-(6-((2-(3,3- dimethylpyrrolidin-1- yl)ethoxy)methyl)pyridin-3- yl)-7-fluoro-1-isopropyl-3- methyl-1H-imidazo[4,5- c]cinnolin-2(3H)-one | 493 [M + H]⁺ |
| 133 | 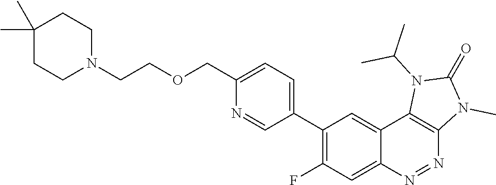 | 8-(6-((2-(4,4- dimethylpiperidin-1- yl)ethoxy)methyl)pyridin-3- yl)-7-fluoro-1-isopropyl-3- methyl-1H-imidazo[4,5- c]cinnolin-2(3H)-one | 507 [M + H]⁺ |
| 134 | 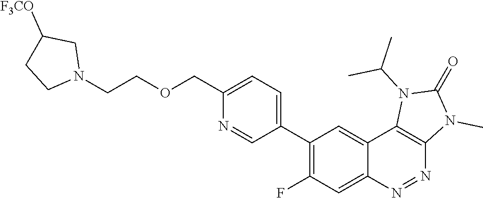 [SMILES: CC(C)n1c(=O)n(C)c2nnc3cc(F)c(-c4ccc(COCCN5CCC(OC(F)(F)F)C5)nc4)cc3c21] | 7-fluoro-1-isopropyl-3-methyl- 8-(6-((2-(3- (trifluoromethoxy)pyrrolidin-1- yl)ethoxy)methyl)pyridin-3- yl)-1,3-dihydro-2H- imidazo[4,5-c]cinnolin-2-one | 549 [M + H]⁺ |

### Example 132

¹H NMR (400 MHz, DMSO-d₆) δ 8.86 (s, 1H), 8.41 (d, J=7.7 Hz, 1H), 8.22 (d, J=11.5 Hz, 1H), 8.20-8.15 (m, 1H), 7.65 (d, J=8.1 Hz, 1H), 5.31-5.21 (m, 1H), 4.68 (s, 2H), 3.68 (t, J=5.9 Hz, 2H), 3.59 (s, 3H), 2.78-2.67 (m, 4H), 2.42 (s, 2H), 1.63 (d, J=6.7 Hz, 6H), 1.54 (t, J=7.1 Hz, 2H), 1.06 (s, 6H).

### Example 133

¹H NMR (400 MHz, DMSO-d₆) δ 8.84 (s, 1H), 8.40 (d, J=7.5 Hz, 1H), 8.21 (d, J=11.4 Hz, 1H), 8.16 (d, J=8.0 Hz, 1H), 7.63 (d, J=8.1 Hz, 1H), 5.32-5.16 (m, 1H), 4.65 (s, 2H), 3.65 (t, J=5.7 Hz, 2H), 3.58 (s, 3H), 2.56 (t, J=5.7 Hz, 2H), 2.43-2.30 (m, 4H), 1.62 (d, J=6.6 Hz, 6H), 1.36-1.25 (m, 4H), 0.87 (s, 6H).

### Example 134

¹H NMR (400 MHz, CDCl₃) δ 8.81 (s, 1H), 8.20-8.13 (m, 2H), 8.01 (d, J=7.8 Hz, 1H), 7.62 (d, J=8.0 Hz, 1H), 5.12 (s, 1H), 4.86 (d, J=16.1 Hz, 2H), 3.82 (s, 1H), 3.74 (s, 3H), 3.21 (s, 1H), 3.03-2.61 (m, 8H), 2.33 (d, J=6.9 Hz, 1H), 2.10 (s, 1H), 1.74 (d, J=6.9 Hz, 6H).

### Example 135

### (R)-8-(6-(1-(2-(5-azaspiro[2.4]heptan-5-yl)ethoxy)ethyl)pyridin-3-yl)-7-fluoro-3-methyl-1-(tetrahydro-2H-pyran-4-yl)-1H-imidazo[4,5-c]cinnolin-2(3H)-one

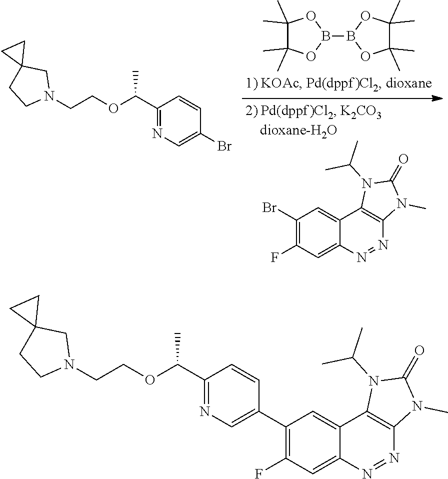

### Step 1: (R)-8-(6-(1-(2-(5-azaspiro[2.4]heptan-5-yl)ethoxy)ethyl)pyridin-3-yl)-7-fluoro-3-methyl-1-(tetrahydro-2H-pyran-4-yl)-1H-imidazo[4,5-c]cinnolin-2(3H)-one

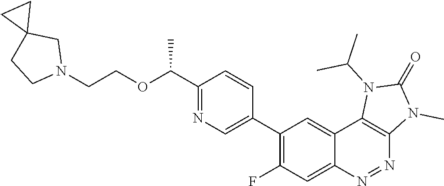 [SMILES: CC(C)n1c(=O)n(C)c2nnc3cc(F)c(-c4ccc([C@@H](C)OCCN5CCC6(CC6)C5)nc4)cc3c21]

The crude product was prepared in a similar fashion to Example 1, which was purified by flash chromatography (DCM:MeOH=9:1) to give the desired product (30.0 mg, 18.4% yield for two steps) as a yellow solid. ¹H NMR (400 MHz, DMSO-d₆) δ 8.88 (s, 1H), 8.44 (d, J=7.7 Hz, 1H), 8.29-8.17 (m, 2H), 7.68 (d, J=8.1 Hz, 1H), 5.33-5.22 (m, 1H), 4.66 (q, J=6.5 Hz, 1H), 3.70-3.64 (m, 1H), 3.60 (s, 3H), 3.58-3.54 (m, 1H), 3.12-3.00 (m, 4H), 2.87 (s, 2H), 1.84 (t, J=7.1 Hz, 2H), 1.64 (d, J=6.7 Hz, 6H), 1.48 (d, J=6.5 Hz, 3H), 0.68-0.53 (m, 4H). LC-MS (ESI) m z: 505 [M+H]⁺.

The following compounds were prepared according to the above described methods using different starting materials.

| Ex # | Structure | Name | MS m/z |
|---|---|---|---|
| 136 | 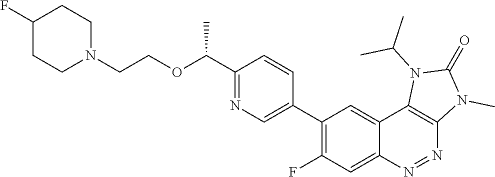 [SMILES: CC(C)n1c(=O)n(C)c2nnc3cc(F)c(-c4ccc([C@@H](C)OCCN5CCC(F)CC5)nc4)cc3c21] | (R)-7-fluoro-8-(6-(1-(2-(4- fluoropiperidin-1- yl)ethoxy)ethyl)pyridin-3-yl)- 1-isopropyl-3-methyl-1,3- dihydro-2H-imidazo[4,5- c]cinnolin-2-one | 511 [M + H]⁺ |
| 137 | 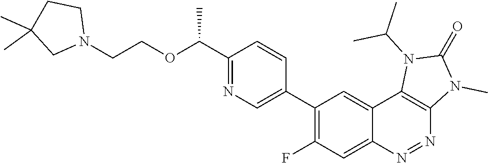 | (R)-8-(6-(1-(2-(3,3- dimethylpyrrolidin-1- yl)ethoxy)ethyl)pyridin-3-yl)- 7-fluoro-1-isopropyl-3- methyl-1H-imidazo[4,5- c]cinnolin-2(3H)-one | 507 [M + H]⁺ |
| 138 | 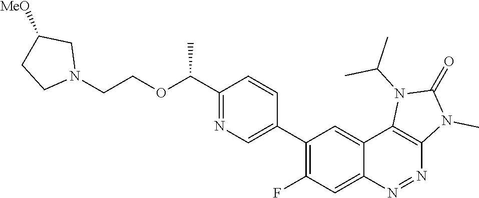 [SMILES: CO[C@H]1CCN(CCO[C@H](C)c2ccc(-c3cc4c(cc3F)nnc3c4n(C(C)C)c(=O)n3C)cn2)C1] | 7-fluoro-1-isopropyl-8-(6- ((R)-1-(2-((S)-3- methoxypyrrolidin-1- yl)ethoxy)ethyl)pyridin-3-yl)- 3-methyl-1H-imidazo[4,5- c]cinnolin-2(3H)-one | 509 [M + H]⁺ |
| 139 | 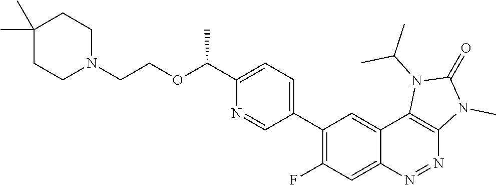 | (R)-8-(6-(1-(2-(4,4- dimethylpiperidin-1- yl)ethoxy)ethyl)pyridin-3-yl)- 7-fluoro-1-isopropyl-3- methyl-1H-imidazo[4,5- c]cinnolin-2(3H)-one | 521 [M + H]⁺ |
| 140 | 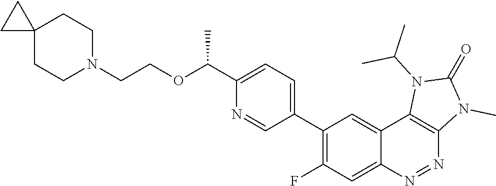 [SMILES: CC(C)n1c(=O)n(C)c2nnc3cc(F)c(-c4ccc([C@@H](C)OCCN5CCC6(CC5)CC6)nc4)cc3c21] | (R)-8-(6-(1-(2-(6- azaspiro[2.5]octan-6- yl)ethoxy)ethyl)pyridin-3-yl)- 7-fluoro-1-isopropyl-3- methyl-1H-imidazo[4,5- c]cinnolin-2(3H)-one | 519 [M + H]⁺ |
| 141 | 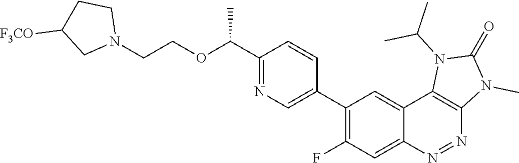 [SMILES: CC(C)n1c(=O)n(C)c2nnc3cc(F)c(-c4ccc([C@@H](C)OCCN5CCC(OC(F)(F)F)C5)nc4)cc3c21] | 7-fluoro-1-isopropyl-3- methyl-8-(6-((1R)-1-(2-(3- (trifluoromethoxy)pyrrolidin- 1-yl)ethoxy)ethyl)pyridin-3- yl)-1H-imidazo[4,5- c]cinnolin-2(3H)-one | 563 [M + H]⁺ |
| 142 | 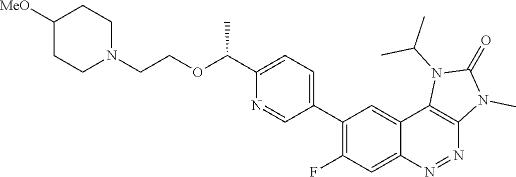 [SMILES: COC1CCN(CCO[C@H](C)c2ccc(-c3cc4c(cc3F)nnc3c4n(C(C)C)c(=O)n3C)cn2)CC1] | (R)-7-fluoro-1-isopropyl-8- (6-(1-(2-(4- methoxypiperidin-1- yl)ethoxy)ethyl)pyridin-3-yl)- 3-methyl-1H-imidazo[4,5- c]cinnolin-2(3H)-one | 523 [M + H]⁺ |
| 143 | 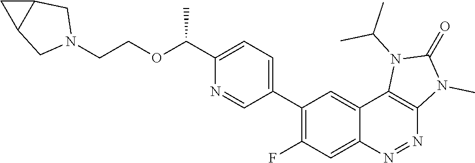 [SMILES: CC(C)n1c(=O)n(C)c2nnc3cc(F)c(-c4ccc([C@@H](C)OCCN5CC6CC6C5)nc4)cc3c21] | 8-(6-((1R)-1-(2-(3- azabicyclo[3.1.0]hexan-3- yl)ethoxy)ethyl)pyridin-3-yl)- 7-fluoro-1-isopropyl-3- methyl-1H-imidazo[4,5- c]cinnolin-2(3H)-one | 491 [M + H]⁺ |
| 144 | 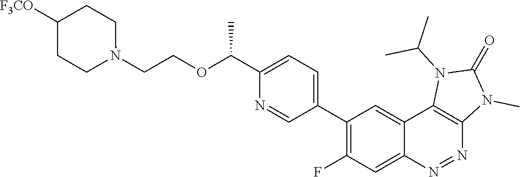 [SMILES: CC(C)n1c(=O)n(C)c2nnc3cc(F)c(-c4ccc([C@@H](C)OCCN5CCC(OC(F)(F)F)CC5)nc4)cc3c21] | (R)-7-fluoro-1-isopropyl-3- methyl-8-(6-(1-(2-(4- (trifluoromethoxy)piperidin- 1-yl)ethoxy)ethyl)pyridin-3- yl)-1H-imidazo[4,5- c]cinnolin-2(3H)-one | 577 [M + H]⁺ |
| 145 | 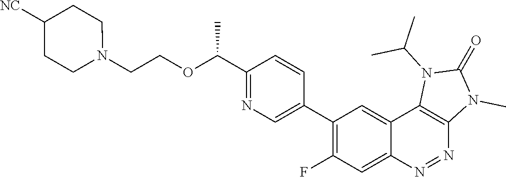 [SMILES: CC(C)n1c(=O)n(C)c2nnc3cc(F)c(-c4ccc([C@@H](C)OCCN5CCC(C#N)CC5)nc4)cc3c21] | (R)-1-(2-(1-(5-(7-fluoro-1- isopropyl-3-methyl-2-oxo- 2,3-dihydro-1H-imidazo[4,5- c]cinnolin-8-yl)pyridin-2- yl)ethoxy)ethyl)piperidine-4- carbonitrile | 518 [M + H]⁺ |
| 146 | 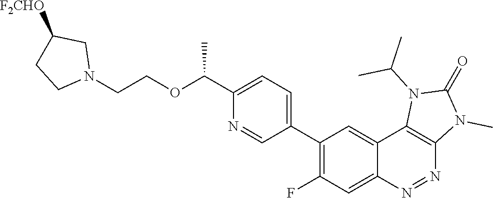 | 8-(6-((R)-1-(2-((R)-3- (difluoromethoxy)pyrrolidin- 1-yl)ethoxy)ethyl)pyridin-3- yl)-7-fluoro-1-isopropyl-3- methyl-1H-imidazo[4,5- c]cinnolin-2(3H)-one | 545 [M + H]⁺ |
| 147 | 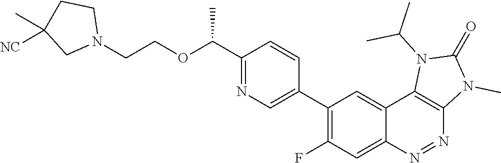 | 1-(2-((R)-1-(5-(7-fluoro-1- isopropyl-3-methyl-2-oxo- 2,3-dihydro-1H-imidazo[4,5- c]cinnolin-8-yl)pyridin-2- yl)ethoxy)ethyl)-3- methylpyrrolidine-3- carbonitrile | 518 [M + H]⁺ |

### Example 136

¹H NMR (400 MHz, DMSO-d₆) δ 8.86 (s, 1H), 8.43 (d, J=7.7 Hz, 1H), 8.26-8.15 (m, 2H), 7.66 (d, J=8.1 Hz, 1H), 5.32-5.21 (m, 1H), 4.75-4.54 (m, 2H), 3.65-3.53 (m, 4H), 3.51-3.42 (m, 1H), 2.62-2.53 (m, 4H), 2.37-2.28 (m, 2H), 1.91-1.77 (m, 2H), 1.74-1.60 (m, 8H), 1.44 (d, J=6.5 Hz, 3H).

### Example 137

¹H NMR (400 MHz, DMSO-d₆) δ 8.87 (s, 1H), 8.43 (d, J=7.7 Hz, 1H), 8.23-8.14 (m, 2H), 7.66 (d, J=8.1 Hz, 1H), 5.32-5.22 (m, 1H), 4.61 (q, J=6.5 Hz, 1H), 3.59 (s, 3H), 3.57-3.53 (m, 1H), 3.49-3.45 (m, 1H), 2.75-2.61 (m, 4H), 2.37 (s, 2H), 1.64 (d, J=6.7 Hz, 6H), 1.52 (t, J=7.1 Hz, 2H), 1.45 (d, J=6.5 Hz, 3H), 1.04 (d, J=2.2 Hz, 6H).

### Example 138

¹H NMR (400 MHz, DMSO-d₆) δ 8.87 (s, 1H), 8.44 (d, J=7.7 Hz, 1H), 8.23 (d, J=11.5 Hz, 1H), 8.21-8.15 (m, 2H), 7.65 (d, J=8.1 Hz, 1H), 5.32-5.22 (m, 1H), 4.61 (q, J=6.5 Hz, 1H), 3.90-3.83 (m, 1H), 3.59 (s, 3H), 3.57-3.54 (m, 2H), 3.50-3.45 (m, 2H), 2.76-2.53 (m, 6H), 2.50-2.45 (m, 1H), 2.01-1.91 (m, 1H), 1.69-1.59 (m, 7H), 1.45 (d, J=6.5 Hz, 3H).

### Example 139

¹H NMR (400 MHz, DMSO-d₆) δ 8.85 (s, 1H), 8.42 (d, J=7.5 Hz, 1H), 8.24-8.13 (m, 2H), 7.65 (d, J=8.1 Hz, 1H), 5.31-5.17 (m, 1H), 4.64-4.54 (m, 1H), 3.61-3.52 (m, 4H), 3.49-3.44 (m, 1H), 2.59-2.51 (m, 2H), 2.42-2.31 (m, 4H), 1.62 (d, J=6.5 Hz, 6H), 1.43 (d, J=6.3 Hz, 3H), 1.33-1.25 (m, 4H), 0.87 (s, 6H).

### Example 140

¹H NMR (400 MHz, DMSO-d₆) δ 8.87 (s, 1H), 8.44 (d, J=7.7 Hz, 1H), 8.22-8.14 (m, 2H), 7.68 (d, J=8.1 Hz, 1H), 5.33-5.22 (m, 1H), 4.62 (q, J=6.5 Hz, 1H), 3.64-3.61 (m, 1H), 3.60 (s, 3H), 3.54-3.51 (m, 1H), 2.71-2.60 (m, 2H), 2.56-2.52 (m, 4H), 1.64 (d, J=6.7 Hz, 6H), 1.46 (d, J=6.5 Hz, 3H), 1.35 (s, 4H), 0.26 (s, 4H).

### Example 141

¹H NMR (400 MHz, DMSO-d₆) δ 8.87 (s, 1H), 8.44 (d, J=7.7 Hz, 1H), 8.25-8.12 (m, 2H), 7.69-7.62 (m, 1H), 5.31-5.23 (m, 1H), 4.97-4.88 (m, 1H), 4.61 (q, J=6.3 Hz, 1H), 3.62-3.54 (m, 4H), 3.51-3.44 (m, 1H), 2.85-2.57 (m, 5H), 2.41-2.32 (m, 1H), 2.28-2.18 (m, 1H), 1.89-1.80 (m, 1H), 1.63 (d, J=6.7 Hz, 6H), 1.45 (d, J=6.5 Hz, 3H).

### Example 142

¹H NMR (400 MHz, DMSO-d₆) δ 8.85 (s, 1H), 8.42 (d, J=7.4 Hz, 1H), 8.21-8.13 (m, 2H), 7.65 (d, J=8.0 Hz, 1H), 5.30-5.20 (m, 1H), 4.64-4.55 (m, 1H), 3.58 (s, 3H), 3.54-3.49 (m, 1H), 3.22-3.10 (m, 5H), 2.78-2.69 (m, 2H), 2.62-2.54 (m, 2H), 2.25-2.12 (m, 2H), 1.85-1.77 (m, 2H), 1.62 (d, J=6.4 Hz, 6H), 1.48-1.35 (m, 5H).

### Example 143

¹H NMR (400 MHz, DMSO-d₆) δ 8.84 (s, 1H), 8.42 (d, J=7.6 Hz, 1H), 8.21 (d, J=11.5 Hz, 1H), 8.16 (d, J=8.1 Hz, 1H), 7.62 (d, J=8.1 Hz, 1H), 5.30-5.20 (m, 1H), 4.57 (q, J=6.4 Hz, 1H), 3.58 (s, 3H), 3.53-3.43 (m, 2H), 2.93 (t, J=8.1 Hz, 2H), 2.67-2.55 (m, 2H), 2.36-2.24 (m, 2H), 1.62 (d, J=6.6 Hz, 6H), 1.42 (d, J=6.5 Hz, 3H), 1.36-1.27 (m, 2H), 0.60-0.53 (m, 1H), 0.32-0.22 (m, 1H).

### Example 144

¹H NMR (400 MHz, CDCl₃) δ 8.75 (s, 1H), 8.16-8.08 (m, 2H), 7.99-7.92 (m, 1H), 7.55 (d, J=8.2 Hz, 1H), 5.13-5.02 (m, 1H), 4.65-4.55 (m, 1H), 4.38-4.22 (m, 1H), 3.75-3.54 (m, 5H), 2.96-2.59 (m, 4H), 1.93-1.78 (m, 2H), 1.70 (d, J=6.9 Hz, 6H), 1.50 (d, J=6.6 Hz, 3H), 1.23-1.14 (m, 2H), 0.86-0.71 (m, 2H).

### Example 145

¹H NMR (400 MHz, DMSO) δ 8.87 (s, 1H), 8.44 (d, J=7.7 Hz, 1H), 8.30 (s, 1H), 8.26-8.15 (m, 2H), 7.66 (d, J=8.1 Hz, 1H), 5.30-5.20 (m, 1H), 4.60 (q, J=6.5 Hz, 1H), 3.59 (s, 3H), 3.58-3.53 (m, 1H), 3.50-3.46 (m, 1H), 2.90-2.81 (m, 1H), 2.63-2.52 (m, 4H), 2.38-2.27 (m, 2H), 1.91-1.79 (m, 2H), 1.74-1.59 (m, 8H), 1.44 (d, J=6.5 Hz, 3H).

### Example 146

¹H NMR (400 MHz, DMSO-d₆) δ 8.87 (s, 1H), 8.44 (d, J=7.7 Hz, 1H), 8.30-8.21 (m, 1H), 8.20-8.16 (m, 1H), 7.66 (d, J=8.1 Hz, 1H), 6.66 (t, J=76.0 Hz, 1H), 5.33-5.20 (m, 1H), 4.71-4.55 (m, 2H), 3.60 (s, 3H), 3.58-3.53 (m, 2H), 2.76-2.64 (m, 4H), 2.62-2.55 (m, 1H), 2.42-2.36 (m, 1H), 2.19-2.10 (m, 1H), 1.80-1.70 (m, 1H), 1.63 (d, J=6.7 Hz, 6H), 1.45 (d, J=6.5 Hz, 3H).

### Example 147

¹H NMR (400 MHz, DMSO-d₆) δ 8.87 (s, 1H), 8.44 (d, J=7.7 Hz, 1H), 8.26-8.15 (m, 2H), 7.68 (dd, J=8.2, 2.1 Hz, 1H), 5.32-5.20 (m, 1H), 4.61 (q, J=6.3 Hz, 1H), 3.59 (s, 3H), 3.58-3.54 (m, 1H), 3.50-3.44 (m, 1H), 3.11-3.01 (m, 1H), 2.85-2.56 (m, 4H), 2.44-2.39 (m, 1H), 2.28-2.18 (m, 1H), 1.85-1.75 (m, 1H), 1.64 (d, J=6.7 Hz, 6H), 1.46 (d, J=6.5 Hz, 3H), 1.40 (d, J=2.2 Hz, 3H).

### Example 148

### (R)-8-(6-(1-(2-(5-azaspiro[2.4]heptan-5-yl)ethoxy)ethyl)pyridin-3-yl)-7-fluoro-3-methyl-1-(tetrahydro-2H-pyran-4-yl)-1H-imidazo[4,5-c]cinnolin-2(3H)-one

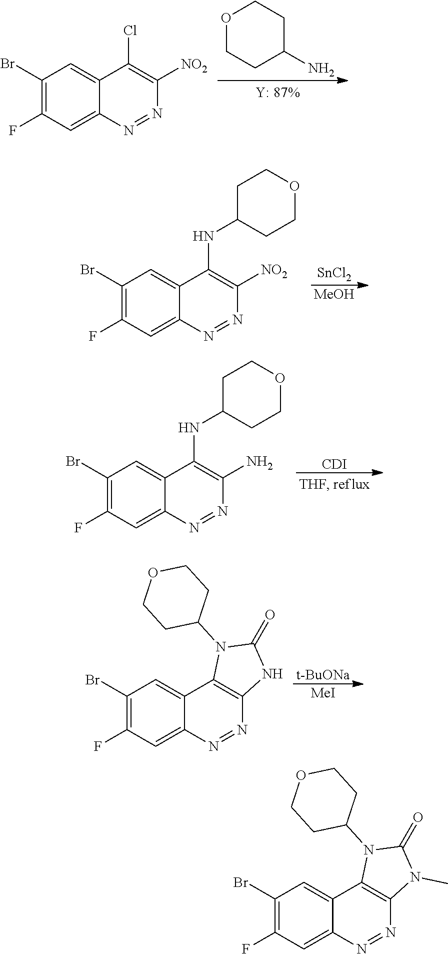

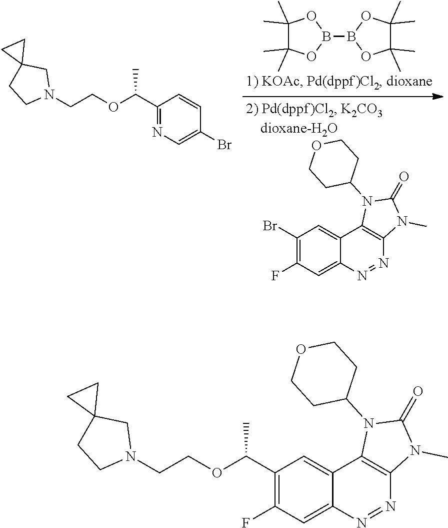

### Step 1: 6-bromo-7-fluoro-3-nitro-N-(tetrahydro-2H-pyran-4-yl)cinnolin-4-amine

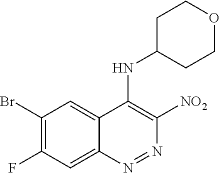 [SMILES: O=[N+]([O-])c1nnc2cc(F)c(Br)cc2c1NC1CCOCC1]

To a solution of 6-bromo-4-chloro-7-fluoro-3-nitrocinnoline (90.0 g, 295 mmol) and Et₃N (63.7 g, 590 mmol) in DCM (1 L) stirred at room temperature, was added tetrahydro-2H-pyran-4-amine (44.8 g, 444 mmol). The resulting mixture was stirred at room temperature for 2 h. The mixture was poured into ice 1 M HCl solution (1 L) and the DCM was evaporated under reduced pressure, yellow solid precipitated out and filtered. The filter residue was triturated with MTBE (300 mL) for 1 h at room temperature and filtered. The filter cake was dried in oven at 55° C. for 16 h to give the desired product (80.0 g, 73.4% yield) as a yellow solid. LC-MS (ESI) m z: 371 [M+H]⁺.

### Step 2: 6-bromo-7-fluoro-N4-(tetrahydro-2H-pyran-4-yl)cinnoline-3,4-diamine

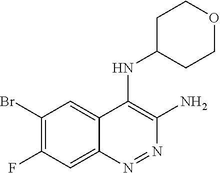 [SMILES: Nc1nnc2cc(F)c(Br)cc2c1NC1CCOCC1]

To a solution of 6-bromo-7-fluoro-3-nitro-N-(tetrahydro-2H-pyran-4-yl)cinnolin-4-amine (80.0 g, 216 mmol) in MeOH (400 mL) was added SnCl₂—H₂O (146 g, 648 mmol) at 0° C. and the reaction mixture was stirred at 60° C. for 2 h. The mixture was adjusted with 50% NaOH solution until pH˜8 below 45° C. and stirred at 55° C. for 45 min. The mixture was filtered and the filtered residue was trituration with MeOH (300 mL) for 1 h at room temperature. After filtration, the combined filtrate was evaporated under reduced pressure. The residue was triturated with MTBE (300 mL) for 1 h at room temperature and filtered. The filter cake was dried in oven at 55° C. for 16 h to give the desired product (65.0 g, 88.6% yield) as a yellow solid. LC-MS (ESI) m z: 341 [M+H]⁺.

### Step 3: 8-bromo-7-fluoro-1-(tetrahydro-2H-pyran-4-yl)-1H-imidazo[4,5-c]cinnolin-2(3H)-one

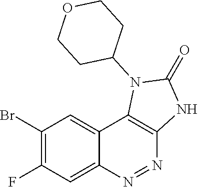 [SMILES: O=c1[nH]c2nnc3cc(F)c(Br)cc3c2n1C1CCOCC1]

To a solution of 6-bromo-7-fluoro-N4-(tetrahydro-2H-pyran-4-yl)cinnoline-3,4-diamine (35.0 g, 103 mmol) in THE (350 mL) stirred at room temperature, was added CDI (50.1 g, 309 mmol). The resulting mixture was stirred at 65° C. for 2 h. The mixture was evaporated under reduced pressure and the residue was dissolved in water (250 mL). It was adjusted with 1 M HCl solution until pH˜7 and filtered. The filtered residue was dried in oven at 55° C. for 16 h to give the desired product (31.0 g, 82.4% yield). LC-MS (ESI) m z: 367 [M+H]⁺.

### Step 4: 8-bromo-7-fluoro-3-methyl-1-(tetrahydro-2H-pyran-4-yl)-1H-imidazo[4,5-c]cinnolin-2(3H)-one

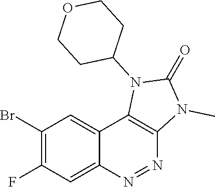 [SMILES: Cn1c(=O)n(C2CCOCC2)c2c3cc(Br)c(F)cc3nnc21]

To a solution of 8-bromo-7-fluoro-1-(tetrahydro-2H-pyran-4-yl)-1H-imidazo[4,5-c]cinnolin-2(3H)-one (31.0 g, 84.7 mmol) in dry DMF (250 mL) was added t-BuONa (9.76 g, 102 mmol) at 0° C. After stirred at 0° C. for 30 min under N₂, to the reaction mixture was added dropwise Mel (14.5 g, 102 mmol) at 10° C.-15° C. and the reaction mixture was stirred at 10° C.-15° C. for 2 h. The mixture was poured into ice water (600 mL) and filtered. The filtered residue was triturated with MeOH (200 mL) at 80° C. for 1 h and filtered. The filter cake was dried under vacuum to give the desired product (25.0 g, 76.8% yield). LC-MS (ESI) m z: 381 [M+H]⁺.

### Step 5: (R)-8-(6-(1-(2-(5-azaspiro[2.4]heptan-5-yl)ethoxy)ethyl)pyridin-3-yl)-7-fluoro-3-methyl-1-(tetrahydro-2H-pyran-4-yl)-1H-imidazo[4,5-c]cinnolin-2(3H)-one

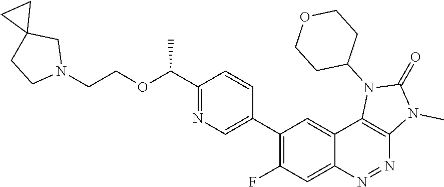 [SMILES: C[C@@H](OCCN1CCC2(CC2)C1)c1ccc(-c2cc3c(cc2F)nnc2c3n(C3CCOCC3)c(=O)n2C)cn1]

The crude product was prepared in a similar fashion to Example 1, which was purified by flash chromatography (DCM:MeOH=9:1) to give the desired product (40.0 mg, 22.7% yield for two steps) as a yellow solid. ¹H NMR (400 MHz, DMSO-d₆) δ 8.91 (s, 1H), 8.47 (d, J=7.8 Hz, 1H), 8.29-8.20 (m, 2H), 7.68 (d, J=8.2 Hz, 1H), 5.14-5.01 (m, 1H), 4.61 (q, J=6.6 Hz, 1H), 4.05-3.96 (m, 2H), 3.61 (s, 3H), 3.59-3.56 (m, 2H), 3.51-3.48 (m, 1H), 2.74-2.56 (m, 7H), 2.47 (s, 2H), 1.98-1.90 (m, 2H), 1.71 (t, J=6.8 Hz, 2H), 1.45 (d, J=6.5 Hz, 3H), 0.56-0.44 (m, 4H). LC-MS (ESI) m z: 547 [M+H]⁺.

The following compounds were prepared according to the above described methods using different starting materials.

| | | | MS |
|---|---|---|---|
| Ex # | Structure | Name | m/z |
| 149 | 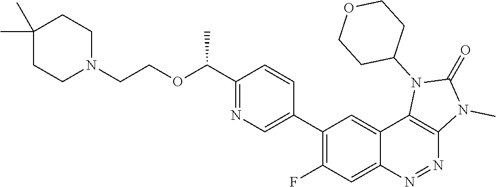 | (R)-8-(6-(1-(2-(4,4- dimethylpiperidin-1- yl)ethoxy)ethyl)pyridin-3-yl)- 7-fluoro-3-methyl-1- (tetrahydro-2H-pyran-4-yl)- 1H-imidazo[4,5-c]cinnolin- 2(3H)-one | 563 [M + H]⁺ |
| 150 | 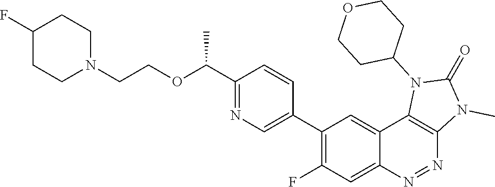 [SMILES: C[C@@H](OCCN1CCC(F)CC1)c1ccc(-c2cc3c(cc2F)nnc2c3n(C3CCOCC3)c(=O)n2C)cn1] | (R)-7-fluoro-8-(6-(1-(2-(4- fluoropiperidin-1- yl)ethoxy)ethyl)pyridin-3-yl)- 3-methyl-1-(tetrahydro-2H- pyran-4-yl)-1H-imidazo[4,5- c]cinnolin-2(3H)-one | 553 [M + H]⁺ |
| 151 | 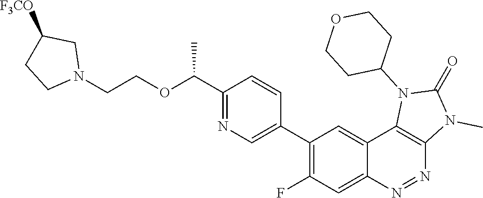 [SMILES: C[C@@H](OCCN1CC[C@@H](OC(F)(F)F)C1)c1ccc(-c2cc3c(cc2F)nnc2c3n(C3CCOCC3)c(=O)n2C)cn1] | 7-fluoro-3-methyl-1- (tetrahydro-2H-pyran-4-yl)-8- (6-((R)-1-(2-((R)-3- (trifluoromethoxy)pyrrolidin- 1-yl)ethoxy)ethyl)pyridin-3- yl)-1,3-dihydro-2H- imidazo[4,5-c]cinnolin-2-one | 605 [M + H]⁺ |
| 152 | 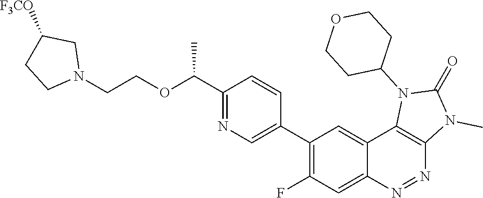 [SMILES: C[C@@H](OCCN1CC[C@H](OC(F)(F)F)C1)c1ccc(-c2cc3c(cc2F)nnc2c3n(C3CCOCC3)c(=O)n2C)cn1] | 7-fluoro-3-methyl-1- (tetrahydro-2H-pyran-4-yl)-8- (6-((R)-1-(2-((S)-3- (trifluoromethoxy)pyrrolidin- 1-yl)ethoxy)ethyl)pyridin-3- yl)-1,3-dihydro-2H- imidazo[4,5-c]cinnolin-2-one | 605 [M + H]⁺ |
| 153 | 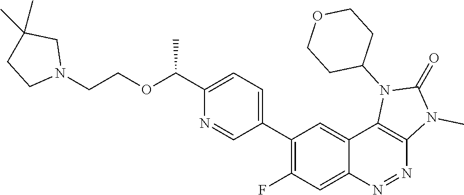 | (R)-8-(6-(1-(2-(3,3- dimethylpyrrolidin-1- yl)ethoxy)ethyl)pyridin-3-yl)- 7-fluoro-3-methyl-1- (tetrahydro-2H-pyran-4-yl)- 1H-imidazo[4,5-c]cinnolin- 2(3H)-one | 549 [M + H]⁺ |
| 154 | 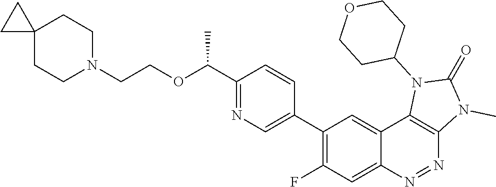 [SMILES: C[C@@H](OCCN1CCC2(CC1)CC2)c1ccc(-c2cc3c(cc2F)nnc2c3n(C3CCOCC3)c(=O)n2C)cn1] | (R)-8-(6-(1-(2-(6- azaspiro[2.5]octan-6- yl)ethoxy)ethyl)pyridin-3-yl)- 7-fluoro-3-methyl-1- (tetrahydro-2H-pyran-4-yl)- 1H-imidazo[4,5-c]cinnolin- 2(3H)-one | 561 [M + H]⁺ |
| 155 | 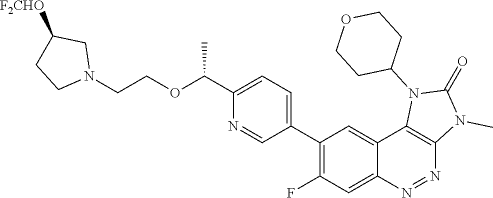 | 8-(6-((R)-1-(2-((R)-3- (difluoromethoxy)pyrrolidin- 1-yl)ethoxy)ethyl)pyridin-3- yl)-7-fluoro-3-methyl-1- (tetrahydro-2H-pyran-4-yl)- 1H-imidazo[4,5-c]cinnolin- 2(3H)-one | 587 [M + H]⁺ |
| 156 | 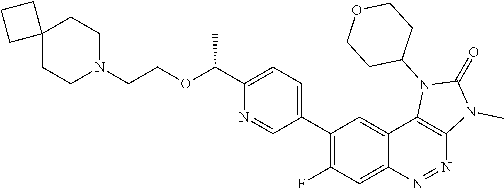 [SMILES: C[C@@H](OCCN1CCC2(CCC2)CC1)c1ccc(-c2cc3c(cc2F)nnc2c3n(C3CCOCC3)c(=O)n2C)cn1] | (R)-8-(6-(1-(2-(7- azaspiro[3.5]nonan-7- yl)ethoxy)ethyl)pyridin-3-yl)- 7-fluoro-3-methyl-1- (tetrahydro-2H-pyran-4-yl)- 1H-imidazo[4,5-c]cinnolin- 2(3H)-one | 575 [M + H]⁺ |
| 157 | 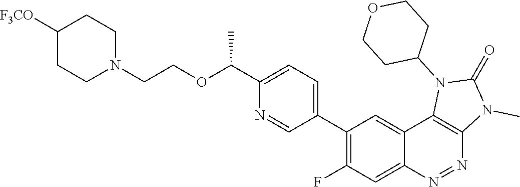 [SMILES: C[C@@H](OCCN1CCC(OC(F)(F)F)CC1)c1ccc(-c2cc3c(cc2F)nnc2c3n(C3CCOCC3)c(=O)n2C)cn1] | (R)-7-fluoro-3-methyl-1- (tetrahydro-2H-pyran-4-yl)-8- (6-(1-(2-(4- (trifluoromethoxy)piperidin- 1-yl)ethoxy)ethyl)pyridin-3- yl)-1H-imidazo[4,5- c]cinnolin-2(3H)-one | 601 [M + H]⁺ |
| 158 | 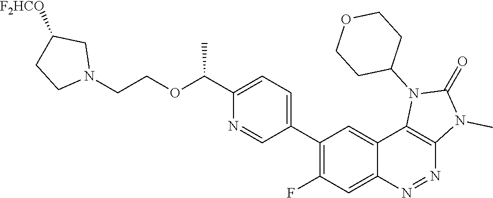 [SMILES: C[C@@H](OCCN1CC[C@H](OC(F)F)C1)c1ccc(-c2cc3c(cc2F)nnc2c3n(C3CCOCC3)c(=O)n2C)cn1] | 8-(6-((R)-1-(2-((S)-3- (difluoromethoxy)pyrrolidin- 1-yl)ethoxy)ethyl)pyridin-3- yl)-7-fluoro-3-methyl-1- (tetrahydro-2H-pyran-4-yl)- 1,3-dihydro-2H-imidazo[4,5- c]cinnolin-2-one | 569 [M + H]⁺ |

### Example 149

¹H NMR (400 MHz, DMSO-d₆) δ 8.91 (s, 1H), 8.53-8.43 (m, 2H), 8.29-8.23 (m, 1H), 7.69 (d, J=8.2 Hz, 1H), 5.13-5.01 (m, 1H), 4.61 (q, J=6.3 Hz, 1H), 4.07-3.96 (m, 2H), 3.65-3.54 (m, 6H), 3.49-3.45 (m, 1H), 2.70-2.53 (m, 4H), 2.40-2.32 (m, 4H), 1.99-1.90 (m, 2H), 1.45 (d, J=6.5 Hz, 3H), 1.34-1.27 (m, 4H), 0.88 (s, 6H).

### Example 150

¹H NMR (400 MHz, DMSO-d₆) δ 8.91 (s, 1H), 8.47 (d, J=7.7 Hz, 1H), 8.30-8.19 (m, 2H), 7.69 (d, J=8.2 Hz, 1H), 5.13-5.01 (m, 1H), 4.74-4.56 (m, 2H), 4.05-3.97 (m, 2H), 3.62-3.55 (m, 6H), 3.51-3.46 (m, 1H), 2.71-2.53 (m, 7H), 2.38-2.29 (m, 2H), 1.99-1.63 (m, 7H), 1.45 (d, J=6.5 Hz, 3H).

### Example 153

¹H NMR (400 MHz, DMSO-d₆) δ 8.90 (s, 1H), 8.45 (d, J=7.6 Hz, 1H), 8.27-8.16 (m, 2H), 7.68 (d, J=8.1 Hz, 1H), 5.12-5.00 (m, 1H), 4.67-4.53 (m, 1H), 4.08-3.95 (m, 2H), 3.59 (s, 3H), 3.58-3.52 (m, 4H), 2.76-2.56 (m, 6H), 2.36 (s, 2H), 1.99-1.88 (m, 2H), 1.51 (t, J=6.9 Hz, 2H), 1.44 (d, J=6.4 Hz, 3H), 1.03 (s, 6H).

### Example 154

¹H NMR (400 MHz, DMSO-d₆) δ 8.67 (s, 1H), 8.23 (d, J=7.7 Hz, 1H), 8.02-7.94 (m, 2H), 7.46 (d, J=8.2 Hz, 1H), 4.89-4.77 (m, 1H), 4.43-4.32 (m, 1H), 3.81-3.69 (m, 2H), 3.40-3.31 (m, 7H), 3.29-3.23 (m, 1H), 2.47-2.30 (m, 7H), 2.24-2.17 (m, 4H), 1.75-1.66 (m, 2H), 1.21 (d, J=6.5 Hz, 3H), 1.09 (s, 4H).

### Example 155

¹H NMR (400 MHz, DMSO-d₆) δ 8.89 (s, 1H), 8.45 (d, J=7.9 Hz, 1H), 8.27-8.16 (m, 2H), 7.66 (d, J=8.6 Hz, 1H), 6.64 (t, J=76.0 Hz, 1H), 5.09-5.00 (m, 1H), 4.70-4.55 (m, 2H), 4.03-3.94 (m, 2H), 3.64-3.44 (m, 7H), 2.76-2.56 (m, 7H), 2.43-2.33 (m, 1H), 2.17-2.07 (m, 1H), 1.96-1.88 (m, 2H), 1.77-1.68 (m, 1H), 1.44 (d, J=6.4 Hz, 3H).

### Example 156

¹H NMR (400 MHz, DMSO-d₆) δ 8.89 (s, 1H), 8.44 (d, J=7.8 Hz, 1H), 8.27-8.17 (m, 2H), 7.67 (d, J=8.3 Hz, 1H), 5.11-5.00 (m, 1H), 4.59 (q, J=6.5 Hz, 1H), 4.07-3.94 (m, 2H), 3.63-3.52 (m, 6H), 3.49-3.42 (m, 2H), 2.69-2.57 (m, 2H), 2.47-2.40 (m, 1H), 2.36-2.18 (m, 4H), 1.98-1.89 (m, 2H), 1.84-1.75 (m, 2H), 1.71-1.63 (m, 4H), 1.54-1.46 (m, 4H), 1.43 (d, J=6.5 Hz, 3H).

### Example 157

¹H NMR (400 MHz, DMSO-d₆) δ 8.91 (s, 1H), 8.47 (d, J=7.7 Hz, 1H), 8.29-8.21 (m, 2H), 7.69 (d, J=8.2 Hz, 1H), 5.11-5.01 (m, 1H), 4.61 (q, J=6.5 Hz, 1H), 4.46-4.36 (m, 1H), 4.04-3.95 (m, 2H), 3.61 (s, 3H), 3.60-3.54 (m, 2H), 3.50-3.44 (m, 1H), 2.74-2.53 (m, 7H), 2.33-2.23 (m, 2H), 1.98-1.87 (m, 4H), 1.75-1.63 (m, 2H), 1.45 (d, J=6.5 Hz, 3H).

### Example 158

¹H NMR (400 MHz, DMSO-d₆) δ 8.91 (s, 1H), 8.46 (d, J=7.6 Hz, 1H), 8.28-8.20 (m, 2H), 7.67 (d, J=8.1 Hz, 1H), 6.65 (t, J=76.0 Hz, 1H), 5.11-5.00 (m, 1H), 4.71-4.56 (m, 2H), 4.04-3.93 (m, 2H), 3.63-3.54 (m, 8H), 2.77-2.57 (m, 6H), 2.42-2.30 (m, 1H), 2.22-2.07 (m, 1H), 1.99-1.89 (m, 2H), 1.78-1.70 (m, 1H), 1.45 (d, J=6.5 Hz, 3H).

### Example 159

### 8-(6-((2-(5-azaspiro[2.4]heptan-5-yl)ethoxy)methyl)pyridin-3-yl)-1-(trans-3-methoxycyclobutyl)-3-methyl-1H-imidazo[4,5-c]cinnolin-2(3H)-one

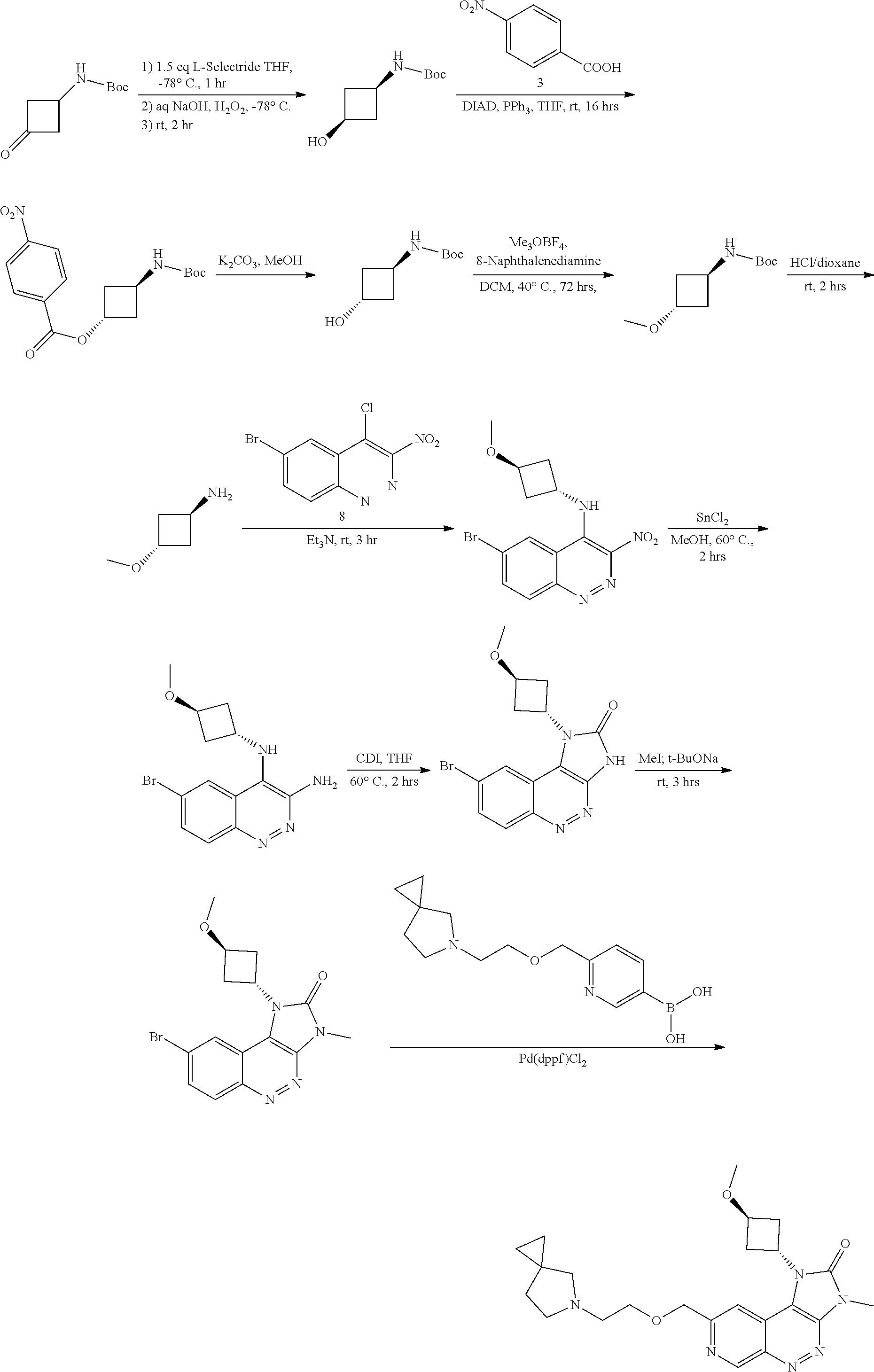

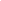

### Step 1: tert-butyl (cis-3-hydroxycyclobutyl)carbamate

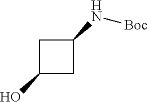 [SMILES: CC(C)(C)OC(=O)N[C@H]1C[C@@H](O)C1]

A solution of tert-butyl (3-oxocyclobutyl)carbamate (10.0 g, 54.0 mmol) in dry tetrahydrofuran (200 mL) was stirred at −78° C. for 10 min under N₂. To the reaction mixture was added dropwise 1 N Lithium triisobutylhydroborate (81.0 mL, 81.0 mmol) at −78° C. and the reaction mixture was stirred at −78° C. for 2 h. The reaction mixture was added dropwise 2N NaOH solution (40 mL) at −45° C. followed by H₂O₂ (30 mL) at −45° C. The resulting mixture was warmed to room temperature and extracted with ethyl acetate (200 mL×2). The combined organic layers were washed with sodium hydrogen sulfite solution (50 mL) and brine (200 mL), dried over anhydrous Na₂SO₄ and filtered, the filtrate was concentrated under reduced pressure. The residue was purified by chromatography on silica gel (PE:EA=5:1 to 1:1) to give the desired product (8.50 g, 84.1% yield) as light oil.

### Step 2: (trans)-3-((tert-butoxycarbonyl)amino)cyclobutyl 4-nitrobenzoate

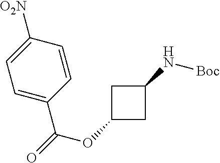 [SMILES: CC(C)(C)OC(=O)N[C@H]1C[C@H](OC(=O)c2ccc([N+](=O)[O-])cc2)C1]

To a mixture of tert-butyl (trans-3-hydroxycyclobutyl)carbamate (7.00 g, 38.0 mmol) and 4-nitrobenzoic acid (7.00 g, 42.0 mmol) in dry tetrahydrofuran (150 mL) was added triphenylphosphine (15.0 g, 57.0 mmol) followed by diisopropyl azodicarboxylate (14.6 g, 72.0 mmol) at 0° C. under N₂. The resulting mixture was warmed to room temperature slowly and stirred overnight. The reaction mixture was evaporated under reduced pressure and the residue was purified by chromatography on silica gel (PE:EA=20:1 to 5:1) to give the crude product (20.0 g) as a white solid.

### Step 3: tert-butyl (trans-3-hydroxycyclobutyl)carbamate

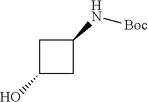 [SMILES: CC(C)(C)OC(=O)N[C@H]1C[C@H](O)C1]

To a solution of trans-3-((tert-butoxycarbonyl)amino)cyclobutyl 4-nitrobenzoate (20.0 g, 60.0 mmol) in H₂O (90 mL) and MeOH (400 mL) was added potassium carbonate (12.5 g, 90.0 mmol) and the reaction mixture was stirred at 70° C. for 1 h. The MeOH was evaporated under reduced pressure and the residue was extracted with ethyl acetate (150 mL×3). The combined organic layers were washed with brine (150 mL), dried over anhydrous Na₂SO₄ filtered, the filtrate was concentrated under reduced pressure. The residue was purified by chromatography on silica gel (PE:EA=5:1 to 1:1) to give the crude product (9.00 g) as a white solid.

### Step 4: tert-butyl (trans-3-methoxycyclobutyl)carbamate

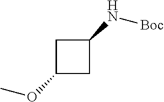

To a solution of tert-butyl (trans-3-hydroxycyclobutyl)carbamate (9.00 g, 48.0 mmol) in DCM (90 mL) was added N1,N1,N8,N8-tetramethylnaphthalene-1,8-diamine (10.3 g, 48.0 mmol) followed by trimethyloxonium tetrafluoroborate (7.60 g, 51.0 mmol) and the reaction mixture was stirred at 40° C. for 72 h under N₂. The reaction mixture was evaporated under reduced pressure and the residue was purified by chromatography on silica gel (PE:EA=15:1 to 3:1) to give the desired product (2.50 g, 27.4% yield for three steps) as a white solid.

### Step 5: trans-3-methoxycyclobutan-1-amine

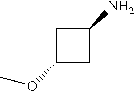

To a solution of tert-butyl (trans-3-methoxycyclobutyl)carbamate (2.50 g) in 1,4-dioxane (20 mL) was added 4 M hydrochloric acid in 1.4-dioxane (20 mL) dropwise at 0° C. and the reaction mixture was stirred at room temperature for 2 h. The reaction mixture was evaporated under reduced pressure to give the crude product (1.50 g) as an off-white solid.

### Step 6: 6-bromo-N-(trans-3-methoxycyclobutyl)-3-nitrocinnolin-4-amine

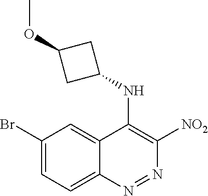 [SMILES: CO[C@H]1C[C@H](Nc2c([N+](=O)[O-])nnc3ccc(Br)cc23)C1]

To a mixture of 6-bromo-4-chloro-3-nitrocinnoline (2.8 g, 9.9 mmol) and propan-2-amine (1.5 g, 15 mmol) in DCM (30 mL) was added TEA (2.8 mL, 20 mmol) and the reaction mixture was stirred at room temperature for 3 h. The mixture was poured into ice 1 M HCl solution (19 mL) and extracted with DCM (50 mL×2). The combined organic layers were washed with brine (80 mL), dried over anhydrous Na₂SO₄ and filtered, the filtrate was concentrated under reduced pressure to give the desired product (3.70 g, 86.1% purity) as a brown solid.

### Step 7: 6-bromo-N⁴-(trans-3-methoxycyclobutyl)cinnoline-3,4-diamine

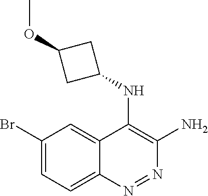 [SMILES: CO[C@H]1C[C@H](Nc2c(N)nnc3ccc(Br)cc23)C1]

To a solution of 6-bromo-N-(trans-3-methoxycyclobutyl)-3-nitrocinnolin-4-amine (3.7 g, 10.5 mmol) in MeOH (40 mL) was added SnCl₂·H₂O (7 g, 31.5 mmol) at 0° C. and the reaction mixture was stirred at 60° C. for 2 h. The mixture was adjusted with 50% NaOH solution until pH˜8 below 45° C. and stirred at 55° C. for 45 min. The mixture was filtered and the filtered residue was trituration with THE (30 mL) for 1 h at room temperature. After filtration, the combined filtrate was evaporated under reduced pressure. The residue was triturated with THE (30 mL) for 1 h at room temperature and filtered. The filtrate was evaporated under reduced pressure to give the desired product (2.00 g, 58.8% yield) as a dark brown solid.

### Step 8: 8-bromo-1-(trans-3-methoxycyclobutyl)-1,3-dihydro-2H-imidazo[4,5-c]cinnolin-2-one

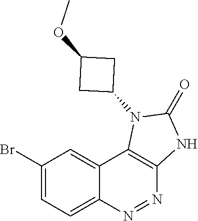 [SMILES: CO[C@H]1C[C@H](n2c(=O)[nH]c3nnc4ccc(Br)cc4c32)C1]

To a solution of 6-bromo-N⁴-(trans-3-methoxycyclobutyl)cinnoline-3,4-diamine (2.00 g, 6.20 mmol) in dry THE (20 mL) was added CDI (3.10 g, 18.6 mmol) and the reaction mixture was stirred at 65° C. for 2 h. The mixture was evaporated under reduced pressure and the residue was dissolved in water (20 mL). It was adjusted with 1 M HCl solution until pH˜7 and filtered. The filtered residue was dried in oven at 55° C. for 16 h to give the desired product (1.20 g, 54.5% yield) as a brown solid.

### Step 9: 8-bromo-1-(trans-3-methoxycyclobutyl)-3-methyl-1,3-dihydro-2H-imidazo[4,5-c]cinnolin-2-one

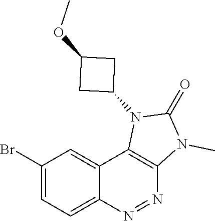 [SMILES: CO[C@H]1C[C@H](n2c(=O)n(C)c3nnc4ccc(Br)cc4c32)C1]

To a solution of 8-bromo-1-(trans-3-methoxycyclobutyl)-1,3-dihydro-2H-imidazo[4,5-c]cinnolin-2-one (1.20 g, 3.50 mmol) in dry DMF (10 mL) was added t-BuONa (404 mg, 4.20 mmol) at 0° C. and stirred at 0° C. for 30 min under N₂. Then to the reaction mixture was added dropwise Mel (1.00 g, 7.00 mmol) at 10° C.-15° C. and the reaction mixture was stirred at 10° C.-15° C. for 2 h. The mixture was poured in ice water (60 mL) and filtered. The filtered residue was triturated with MeOH (20 mL) for 1 h and filtered. The filter cake was dried under vacuum to give the desired product (950 mg, 74.9% yield) a light brown solid.

### Step 10: 8-(6-((2-(5-azaspiro[2.4]heptan-5-yl)ethoxy)methyl)pyridin-3-yl)-1-(trans-3-methoxycyclobutyl)-3-methyl-1,3-dihydro-2H-imidazo[4,5-c]cinnolin-2-one

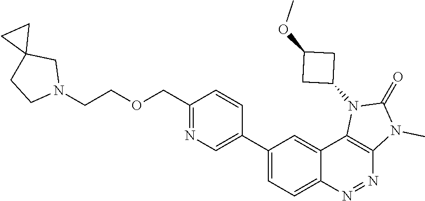 [SMILES: CO[C@H]1C[C@H](n2c(=O)n(C)c3nnc4ccc(-c5ccc(COCCN6CCC7(CC7)C6)nc5)cc4c32)C1]

The crude product was prepared in a similar fashion to Example 1, which was purified by chromatography on silica gel (DCM:MeOH=100:1 to 10:1) to give crude product. It was purified 3 times by flash chromatography (DCM:MeOH=9:1) to give the desired product (50.0 mg, 20% yield for two steps) as an off-white solid. ¹H NMR (400 MHz, DMSO-d₆) δ 9.06 (d, J=2.0 Hz, 1H), 8.41 (d, J=9.1 Hz, 1H), 8.37-8.29 (m, 2H), 8.10 (dd, J=9.1, 1.7 Hz, 1H), 7.63 (d, J=8.1 Hz, 1H), 5.63-5.52 (m, 1H), 4.67 (s, 2H), 4.32-4.23 (m, 1H), 3.66 (t, J=5.9 Hz, 2H), 3.61 (s, 3H), 3.24-3.13 (m, 5H), 2.72-2.57 (m, 6H), 2.48 (s, 2H), 1.72 (t, J=6.8 Hz, 2H), 0.56-0.44 (in, 4H). LC-MS (ESI) m/z: 515 [M+H]⁺.

The following compounds were prepared according to the above described methods using different starting materials.

| Ex | | | MS |
|---|---|---|---|
| # | Structure | Name | m/z |
| 160 | 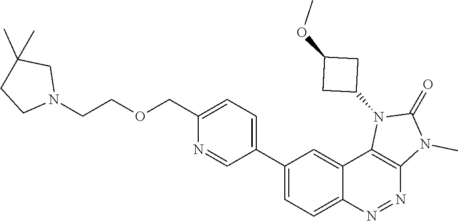 | 8-(6-((2-(3,3- dimethylpyrrolidin-1- yl)ethoxy)methyl)pyridin-3- yl)-1-(trans-3- methoxycyclobutyl)-3- methyl-1H-imidazo[4,5- c]cinnolin-2(3H)-one | 517 [M + H]+ |
| 161 | 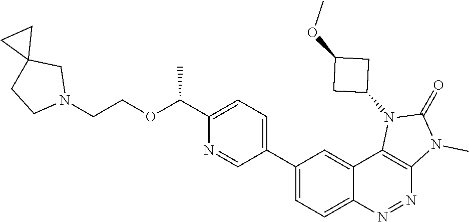 [SMILES: CO[C@H]1C[C@H](n2c(=O)n(C)c3nnc4ccc(-c5ccc([C@@H](C)OCCN6CCC7(CC7)C6)nc5)cc4c32)C1] | 8-(6-((R)-1-(2-(5- azaspiro[2.4]heptan-5- yl)ethoxy)ethyl)pyridin-3-yl)- 1-(trans-3- methoxycyclobutyl)-3- methyl-1H-imidazo[4,5- c]cinnolin-2(3H)-one | 529 [M + H]⁺ |
| 162 | 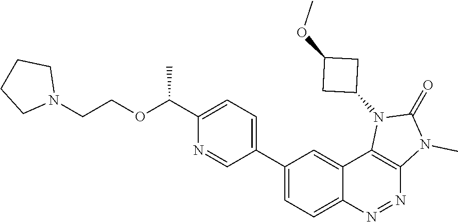 [SMILES: CO[C@H]1C[C@H](n2c(=O)n(C)c3nnc4ccc(-c5ccc([C@@H](C)OCCN6CCCC6)nc5)cc4c32)C1] | 1-(trans-3- methoxycyclobutyl)-3- methyl-8-(6-((R)-1-(2- (pyrrolidin-1- yl)ethoxy)ethyl)pyridin-3-yl)- 1,3-dihydro-2H-imidazo[4,5- c]cinnolin-2-one | 503 [M + H]⁺ |
| 163 | 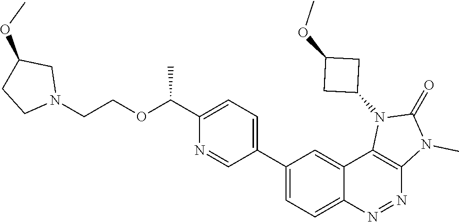 [SMILES: CO[C@@H]1CCN(CCO[C@H](C)c2ccc(-c3ccc4nnc5c(c4c3)n([C@H]3C[C@H](OC)C3)c(=O)n5C)cn2)C1] | 1-(trans-3- methoxycyclobutyl)-8-(6- ((R)-1-(2-((R)-3- methoxypyrrolidin-1- yl)ethoxy)ethyl)pyridin-3-yl)- 3-methyl-1H-imidazo[4,5- c]cinnolin-2(3H)-one | 533 [M + H]⁺ |
| 164 | 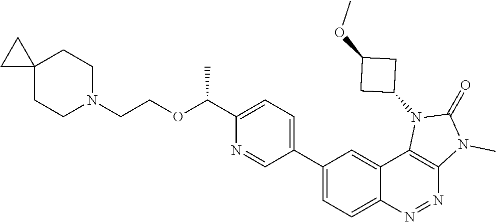 [SMILES: CO[C@H]1C[C@H](n2c(=O)n(C)c3nnc4ccc(-c5ccc([C@@H](C)OCCN6CCC7(CC6)CC7)nc5)cc4c32)C1] | 8-(6-((R)-1-(2-(6- azaspiro[2.5]octan-6- yl)ethoxy)ethyl)pyridin-3-yl)- 1-(trans-3- methoxycyclobutyl)-3- methyl-1H-imidazo[4,5- c]cinnolin-2(3H)-one | 543 [M + H]⁺ |
| 165 | 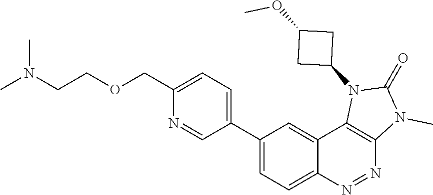 | 8-(6-((2- (dimethylamino)ethoxy)meth yl)pyridin-3-yl)-1-(trans-3- methoxycyclobutyl)-3- methyl-1H-imidazo[4,5- c]cinnolin-2(3H)-one | 463 [M + H]⁺ |
| 166 | 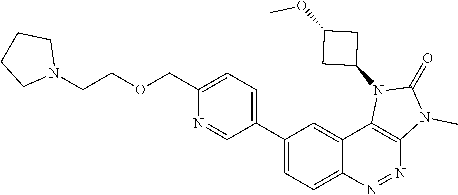 [SMILES: CO[C@H]1C[C@H](n2c(=O)n(C)c3nnc4ccc(-c5ccc(COCCN6CCCC6)nc5)cc4c32)C1] | 1-(trans-3- methoxycyclobutyl)-3- methyl-8-(6-((2-(pyrrolidin- 1-yl)ethoxy)methyl)pyridin- 3-yl)-1H-imidazo[4,5- c]cinnolin-2(3H)-one | 489 [M + H]⁺ |
| 167 | 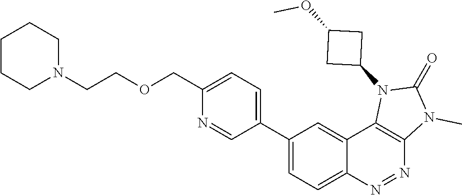 | 1-(trans-3- methoxycyclobutyl)-3- methyl-8-(6-((2-(piperidin-1- yl)ethoxy)methyl)pyridin-3- yl)-1H-imidazo[4,5- c]cinnolin-2(3H)-one | 521 [M + H]⁺ |
| 168 | 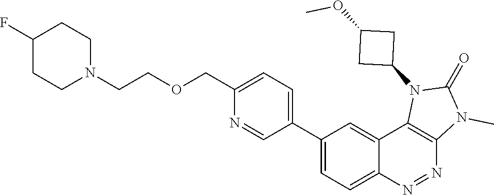 [SMILES: CO[C@H]1C[C@H](n2c(=O)n(C)c3nnc4ccc(-c5ccc(COCCN6CCC(F)CC6)nc5)cc4c32)C1] | 8-(6-((2-(4-fluoropiperidin-1- yl)ethoxy)methyl)pyridin-3- yl)-1-(trans-3- methoxycyclobutyl)-3- methyl-1H-imidazo[4,5- c]cinnolin-2(3H)-one | 521 [M + H]⁺ |
| 169 | 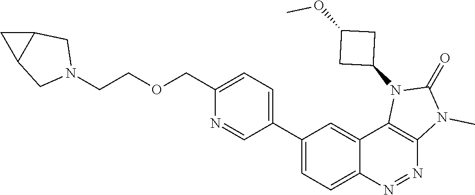 [SMILES: CO[C@H]1C[C@H](n2c(=O)n(C)c3nnc4ccc(-c5ccc(COCCN6CC7CC7C6)nc5)cc4c32)C1] | 8-(6-((2-(3- azabicyclo[3.1.0]hexan-3- yl)ethoxy)methyl)pyridin-3- yl)-1-(trans-3- methoxycyclobutyl)-3- methyl-1H-imidazo[4,5- c]cinnolin-2(3H)-one | 501 [M + H]⁺ |
| 170 | 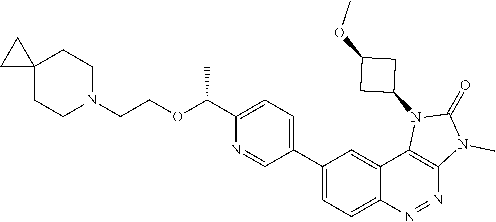 [SMILES: CO[C@H]1C[C@@H](n2c(=O)n(C)c3nnc4ccc(-c5ccc([C@@H](C)OCCN6CCC7(CC6)CC7)nc5)cc4c32)C1] | 8-(6-((R)-1-(2-(6- azaspiro[2.5]octan-6- yl)ethoxy)ethyl)pyridin-3-yl)- 1-(cis-3-methoxycyclobutyl)- 3-methyl-1H-imidazo[4,5- c]cinnolin-2(3H)-one | 543 [M + H]⁺ |
| 171 | 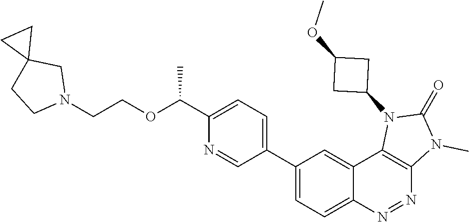 [SMILES: CO[C@H]1C[C@@H](n2c(=O)n(C)c3nnc4ccc(-c5ccc([C@@H](C)OCCN6CCC7(CC7)C6)nc5)cc4c32)C1] | 8-(6-((R)-1-(2-(5- azaspiro[2.4]heptan-5- yl)ethoxy)ethyl)pyridin-3-yl)- 1-(cis-3-methoxycyclobutyl)- 3-methyl-1H-imidazo[4,5- c]cinnolin-2(3H)-one | 529 [M + H]⁺ |
| 172 | 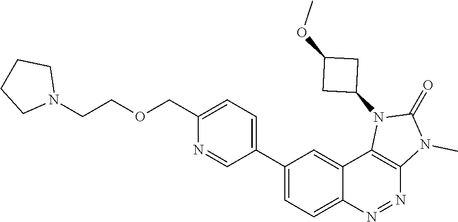 [SMILES: CO[C@H]1C[C@@H](n2c(=O)n(C)c3nnc4ccc(-c5ccc(COCCN6CCCC6)nc5)cc4c32)C1] | 1-(cis-3-methoxycyclobutyl)- 3-methyl-8-(6-((2- (pyrrolidin-1- yl)ethoxy)methyl)pyridin-3- yl)-1H-imidazo[4,5- c]cinnolin-2(3H)-one | 489 [M + H]⁺ |
| 173 | 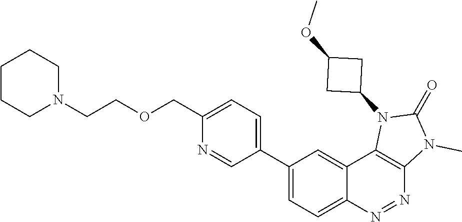 [SMILES: CO[C@H]1C[C@@H](n2c(=O)n(C)c3nnc4ccc(-c5ccc(COCCN6CCCCC6)nc5)cc4c32)C1] | 1-(cis-3-methoxycyclobutyl)- 3-methyl-8-(6-((2-(piperidin- 1-yl)ethoxy)methyl)pyridin- 3-yl)-1H-imidazo[4,5- c]cinnolin-2(3H)-one | 503 [M + H]⁺ |
| 174 | 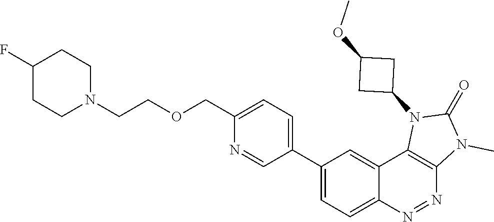 [SMILES: CO[C@H]1C[C@@H](n2c(=O)n(C)c3nnc4ccc(-c5ccc(COCCN6CCC(F)CC6)nc5)cc4c32)C1] | 8-(6-((2-(4-fluoropiperidin-1- yl)ethoxy)methyl)pyridin-3- yl)-1-(cis-3- methoxycyclobutyl)-3- methyl-1H-imidazo[4,5- c]cinnolin-2(3H)-one | |
| 175 | 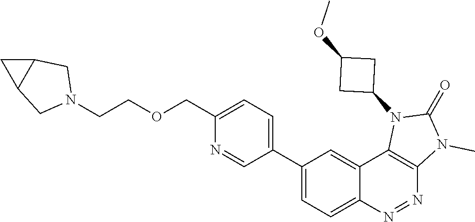 [SMILES: CO[C@H]1C[C@@H](n2c(=O)n(C)c3nnc4ccc(-c5ccc(COCCN6CC7CC7C6)nc5)cc4c32)C1] | 8-(6-((2-(3- azabicyclo[3.1.0]hexan-3- yl)ethoxy)methyl)pyridin-3- yl)-1-(cis-3- methoxycyclobutyl)-3- methyl-1H-imidazo[4,5- c]cinnolin-2(3H)-one | 501 [M + H]⁺ |
| 176 | 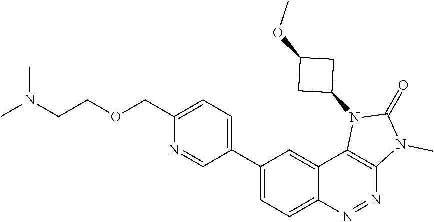 | 8-(6-((2- (dimethylamino)ethoxy)meth yl)pyridin-3-yl)-1-(cis-3- methoxycyclobutyl)-3- methyl-1H-imidazo[4,5- c]cinnolin-2(3H)-one | 463 [M + H]⁺ |

### Example 160

¹H NMR (400 MHz, CDCl₃) δ 8.94 (s, 1H), 8.55 (d, J=9.0 Hz, 1H), 8.21 (s, 1H), 8.06 (d, J=7.5 Hz, 1H), 7.88 (d, J=9.0 Hz, 1H), 7.62 (d, J=7.8 Hz, 1H), 5.58-5.44 (m, 1H), 4.84-4.65 (m, 2H), 4.40-4.29 (m, 1H), 4.25-4.09 (m, 2H), 3.78 (s, 3H), 3.47-3.31 (m, 9H), 2.69-2.59 (m, 2H), 2.10-1.88 (m, 4H), 1.29 (s, 6H).

### Example 161

¹H NMR (400 MHz, DMSO-d₆) δ 9.06 (d, J=1.9 Hz, 1H), 8.42 (d, J=9.1 Hz, 1H), 8.38-8.30 (m, 2H), 8.10 (dd, J=9.1, 1.7 Hz, 1H), 7.65 (d, J=8.2 Hz, 1H), 5.65-5.53 (m, 1H), 4.61 (q, J=6.5 Hz, 1H), 4.33-4.24 (m, 1H), 3.60 (s, 3H), 3.59-3.54 (m, 1H), 3.50-3.46 (m, 1H), 3.22 (s, 3H), 3.21-3.14 (m, 2H), 2.78-2.58 (m, 6H), 2.54 (s, 2H), 1.73 (t, J=6.9 Hz, 2H), 1.45 (d, J=6.5 Hz, 3H), 0.57-0.45 (m, 4H).

### Example 163

¹H NMR (400 MHz, CDCl₃) δ 8.89 (s, 1H), 8.55 (d, J=9.0 Hz, 1H), 8.19 (s, 1H), 8.03 (d, J=8.1 Hz, 1H), 7.88 (d, J=8.9 Hz, 1H), 7.65 (d, J=8.2 Hz, 1H), 5.54-5.45 (m, 1H), 4.71-4.63 (m, 1H), 4.36-4.31 (m, 1H), 4.03-3.96 (m, 1H), 3.81-3.66 (m, 6H), 3.47-3.36 (m, 3H), 3.35 (s, 3H), 3.31 (s, 3H), 3.02-2.79 (m, 4H), 2.71-2.59 (m, 2H), 2.19-2.07 (m, 1H), 2.00-1.88 (m, 1H), 1.57 (d, J=6.5 Hz, 3H).

### Example 164

¹H NMR (400 MHz, DMSO-d₆) δ 9.06 (d, J=2.0 Hz, 1H), 8.42 (d, J=9.1 Hz, 1H), 8.37 (d, J=1.4 Hz, 1H), 8.33 (dd, J=8.2, 2.4 Hz, 1H), 8.10 (dd, J=9.1, 1.7 Hz, 1H), 7.66 (d, J=8.2 Hz, 1H), 5.63-5.54 (m, 1H), 4.61 (q, J=6.5 Hz, 1H), 4.32-4.22 (m, 1H), 3.63-3.54 (m, 4H), 3.52-3.44 (m, 1H), 3.22 (s, 3H), 3.20-3.13 (m, 2H), 2.68-2.53 (m, 4H), 2.47-2.33 (m, 4H), 1.44 (d, J=6.5 Hz, 3H), 1.37-1.24 (m, 4H), 0.23 (s, 4H).

### Example 165

¹H NMR (400 MHz, DMSO-d₆) δ 9.01 (d, J=2.3 Hz, 1H), 8.35 (d, J=9.1 Hz, 1H), 8.31-8.23 (m, 2H), 8.04 (dd, J=9.1, 1.7 Hz, 1H), 7.59 (d, J=8.1 Hz, 1H), 5.57-5.45 (m, 1H), 4.63 (s, 2H), 4.29-4.20 (m, 1H), 3.64 (t, J=5.8 Hz, 2H), 3.57 (s, 3H), 3.24-3.08 (m, 5H), 2.65-2.50 (m, 4H), 2.20 (s, 6H).

### Example 166

¹H NMR (400 MHz, DMSO-d₆) δ 9.03 (s, 1H), 8.39 (d, J=9.1 Hz, 1H), 8.35-8.26 (m, 2H), 8.07 (d, J=9.1 Hz, 1H), 7.61 (d, J=8.2 Hz, 1H), 5.59-5.50 (m, 1H), 4.65 (s, 2H), 4.31-4.20 (m, 1H), 3.66 (t, J=6.0 Hz, 2H), 3.59 (s, 3H), 3.23-3.10 (m, 5H), 2.76-2.67 (m, 2H), 2.65-2.50 (m, 6H), 1.74-1.58 (m, 4H).

### Example 167

¹H NMR (400 MHz, DMSO-d₆) δ 9.03 (d, J=2.3 Hz, 1H), 8.39 (d, J=9.0 Hz, 1H), 8.35-8.25 (m, 2H), 8.07 (d, J=9.0 Hz, 1H), 7.61 (d, J=8.1 Hz, 1H), 5.61-5.48 (m, 1H), 4.64 (s, 2H), 4.31-4.19 (m, 1H), 3.64 (t, J=5.9 Hz, 2H), 3.58 (s, 3H), 3.23-3.10 (m, 5H), 2.65-2.51 (m, 4H), 2.44-2.27 (m, 4H), 1.54-1.42 (m, 4H), 1.41-1.29 (m, 2H).

### Example 168

¹H NMR (400 MHz, DMSO-d₆) δ 9.01 (d, J=2.3 Hz, 1H), 8.36 (d, J=9.1 Hz, 1H), 8.31-8.23 (m, 2H), 8.05 (dd, J=9.1, 1.7 Hz, 1H), 7.60 (d, J=8.1 Hz, 1H), 5.59-5.45 (m, 1H), 4.75-4.53 (m, 3H), 4.30-4.18 (m, 1H), 3.65 (t, J=5.8 Hz, 2H), 3.57 (s, 3H), 3.25-3.09 (m, 5H), 2.68-2.51 (m, 6H), 2.42-2.29 (m, 2H), 1.92-1.75 (m, 2H), 1.75-1.60 (m, 2H).

### Example 169

¹H NMR (400 MHz, DMSO-d₆) δ 9.00 (d, J=2.3 Hz, 1H), 8.35 (d, J=9.1 Hz, 1H), 8.31-8.22 (m, 2H), 8.04 (dd, J=9.1, 1.7 Hz, 1H), 7.57 (d, J=8.1 Hz, 1H), 5.56-5.46 (m, 1H), 4.61 (s, 2H), 4.31-4.18 (m, 1H), 3.63-3.52 (m, 5H), 3.26-3.05 (m, 5H), 2.96 (d, J=8.6 Hz, 2H), 2.71-2.53 (m, 4H), 2.31 (d, J=8.4 Hz, 2H), 1.37-1.28 (m, 2H), 0.60-0.54 (m, 1H), 0.31-0.24 (m, 1H).

### Example 170

¹H NMR (400 MHz, DMSO-d₆) δ 9.06 (d, J=2.0 Hz, 1H), 8.54 (d, J=1.3 Hz, 1H), 8.42 (d, J=9.1 Hz, 1H), 8.34 (dd, J=8.2, 2.4 Hz, 1H), 8.09 (dd, J=9.1, 1.6 Hz, 1H), 7.65 (d, J=8.2 Hz, 1H), 5.19-5.06 (m, 1H), 4.61 (q, J=6.5 Hz, 1H), 3.91-3.82 (m, 1H), 3.62-3.56 (m, 5H), 3.52-3.46 (m, 4H), 3.20 (s, 3H), 3.03-2.95 (m, 2H), 2.87-2.78 (m, 2H), 2.67-2.56 (m, 2H), 1.44 (d, J=6.5 Hz, 3H), 1.38-1.26 (m, 4H), 0.24 (s, 4H).

### Example 171

¹H NMR (400 MHz, DMSO) δ 9.07 (d, J=1.9 Hz, 1H), 8.55 (d, J=1.3 Hz, 1H), 8.43 (d, J=9.1 Hz, 1H), 8.34 (dd, J=8.2, 2.4 Hz, 1H), 8.10 (dd, J=9.1, 1.7 Hz, 1H), 7.64 (d, J=8.2 Hz, 1H), 5.19-5.08 (m, 1H), 4.61 (q, J=6.5 Hz, 1H), 3.93-3.83 (m, 1H), 3.60 (s, 3H), 3.59-3.54 (m, 1H), 3.48-3.44 (m, 1H), 3.20 (s, 3H), 3.05-2.95 (m, 2H), 2.88-2.80 (m, 2H), 2.77-2.64 (m, 4H), 2.53 (s, 2H), 1.73 (t, J=6.9 Hz, 2H), 1.45 (d, J=6.5 Hz, 3H), 0.57-0.46 (m, 4H).

### Example 172

¹H NMR (400 MHz, DMSO-d₆) δ 9.04 (d, J=2.4 Hz, 1H), 8.50 (d, J=1.8 Hz, 1H), 8.40 (d, J=9.0 Hz, 1H), 8.31 (dd, J=8.1, 2.5 Hz, 1H), 8.07 (dd, J=9.1, 1.7 Hz, 1H), 7.60 (d, J=8.2 Hz, 1H), 5.15-5.03 (m, 1H), 4.64 (s, 2H), 3.90-3.80 (m, 1H), 3.66 (t, J=5.9 Hz, 2H), 3.57 (s, 3H), 3.18 (s, 3H), 3.01-2.91 (m, 2H), 2.86-2.77 (m, 2H), 2.70 (t, J=5.9 Hz, 2H), 2.56-2.50 (m, 4H), 1.73-1.60 (m, 4H).

### Example 173

¹H NMR (400 MHz, DMSO-d₆) δ 9.04 (s, 1H), 8.51 (s, 1H), 8.41 (d, J=9.1 Hz, 1H), 8.31 (d, J=8.2 Hz, 1H), 8.08 (d, J=9.1 Hz, 1H), 7.61 (d, J=8.1 Hz, 1H), 5.15-5.04 (m, 1H), 4.64 (s, 2H), 3.92-3.80 (m, 1H), 3.65 (t, J=5.9 Hz, 2H), 3.58 (s, 3H), 3.18 (s, 3H), 3.01-2.92 (m, 2H), 2.86-2.76 (m, 2H), 2.60-2.52 (m, 2H), 2.44-2.32 (m, 4H), 1.57-1.29 (m, 6H).

### Example 174

¹H NMR (400 MHz, DMSO-d₆) δ 9.03 (d, J=2.9 Hz, 1H), 8.48 (s, 1H), 8.39 (dd, J=9.1, 2.8 Hz, 1H), 8.30 (dd, J=8.0, 2.7 Hz, 1H), 8.06 (d, J=9.0 Hz, 1H), 7.60 (d, J=8.1 Hz, 1H), 5.16-5.03 (m, 1H), 4.74-4.53 (m, 3H), 3.91-3.81 (m, 1H), 3.65 (t, J=5.6 Hz, 2H), 3.57 (s, 3H), 3.18 (s, 3H), 3.02-2.91 (m, 2H), 2.87-2.74 (m, 2H), 2.65-2.53 (m, 4H), 2.43-2.29 (m, 2H), 1.89-1.76 (m, 2H), 1.74-1.59 (m, 2H).

### Example 175

¹H NMR (400 MHz, DMSO-d₆) δ 9.04 (d, J=2.3 Hz, 1H), 8.50 (s, 1H), 8.40 (d, J=9.1 Hz, 1H), 8.31 (dd, J=8.1, 2.4 Hz, 1H), 8.07 (d, J=9.1 Hz, 1H), 7.58 (d, J=8.1 Hz, 1H), 5.17-5.02 (m, 1H), 4.63 (s, 2H), 3.91-3.81 (m, 1H), 3.63-3.54 (m, 5H), 3.18 (s, 3H), 3.04-2.91 (m, 4H), 2.87-2.76 (m, 2H), 2.66 (t, J=5.8 Hz, 2H), 2.40-2.29 (m, 2H), 1.36-1.30 (m, 2H), 0.61-0.53 (m, 1H), 0.32-0.24 (m, 1H).

### Example 176

¹H NMR (400 MHz, DMSO-d₆) δ 9.04 (s, 1H), 8.51 (s, 1H), 8.41 (d, J=9.1 Hz, 1H), 8.31 (d, J=8.2 Hz, 1H), 8.08 (d, J=9.1 Hz, 1H), 7.60 (d, J=8.2 Hz, 1H), 5.16-5.05 (m, 1H), 4.64 (s, 2H), 3.90-3.81 (m, 1H), 3.64 (t, J=5.8 Hz, 2H), 3.58 (s, 3H), 3.18 (s, 3H), 3.01-2.91 (m, 2H), 2.86-2.77 (m, 2H), 2.54-2.51 (m, 2H), 2.18 (s, 6H).

The following compounds were prepared according to the above described methods using different starting materials.

| Ex | | | MS |
|---|---|---|---|
| # | Structure | Name | m/z |
| 177 | 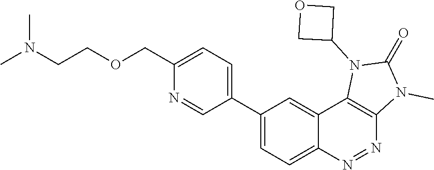 | 8-(6-((2- (dimethylamino)ethoxy) methyl)pyridin-3-yl)- 3-methyl-1- (oxetan-3-yl)-1H- imidazo[4,5-c]cinnolin- 2(3H)-one | 435 [M + H]⁺ |
| 178 | 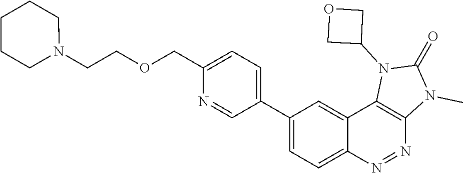 [SMILES: Cn1c(=O)n(C2COC2)c2c3cc(-c4ccc(COCCN5CCCCC5)nc4)ccc3nnc21] | 3-methyl-1-(oxetan-3-yl)-8- (6-((2-(piperidin-1- yl)ethoxy)methyl)pyridin-3- yl)-1H-imidazo[4,5- c]cinnolin-2(3H)-one | 475 [M + H]⁺ |
| 179 | 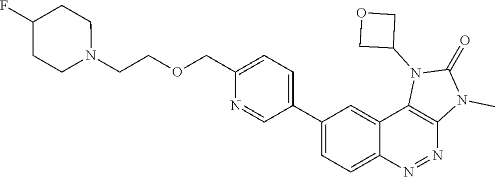 [SMILES: Cn1c(=O)n(C2COC2)c2c3cc(-c4ccc(COCCN5CCC(F)CC5)nc4)ccc3nnc21] | 8-(6-((2-(4-fluoropiperidin-1- yl)ethoxy)methyl)pyridin-3- yl)-3-methyl-1-(oxetan-3-yl)- 1H-imidazo[4,5-c]cinnolin- 2(3H)-one | 493 [M + H]⁺ |
| 180 | 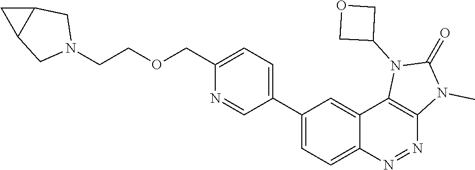 | 8-(6-((2-(4-fluoropiperidin-1- yl)ethoxy)methyl)pyridin-3- yl)-1-((1s,3s)-3- methoxycyclobutyl)-3- methyl-1H-imidazo[4,5- c]cinnolin-2(3H)-one | 521 [M + H]⁺ |
| 181 | 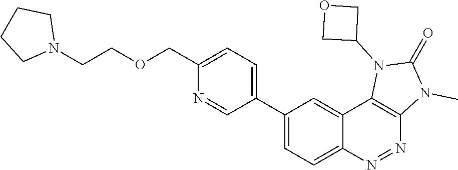 [SMILES: Cn1c(=O)n(C2COC2)c2c3cc(-c4ccc(COCCN5CCCC5)nc4)ccc3nnc21] | 3-methyl-1-(oxetan-3-yl)-8- (6-((2-(pyrrolidin-1- yl)ethoxy) methyl)pyridin-3- yl)-1H-imidazo[4,5- c]cinnolin-2(3H)-one | 461 [M + H]⁺ |

### Example 177

¹H NMR (400 MHz, DMSO-d₆) δ 8.92 (s, 1H), 8.27-8.14 (m, 2H), 8.10 (s, 1H), 7.86 (d, J=9.0, 2.0 Hz, 1H), 7.56 (d, J=8.2 Hz, 1H), 4.82-4.71 (m, 1H), 4.61 (s, 2H), 4.45-4.36 (m, 1H), 4.14-4.04 (m, 1H), 3.79-3.69 (m, 1H), 3.61 (t, J=5.9 Hz, 2H), 3.49-3.44 (m, 1H), 3.41 (s, 3H), 2.47-2.45 (m, 2H), 2.16 (s, 6H).

### Example 178

¹H NMR (400 MHz, DMSO-d₆) δ 8.95 (d, J=1.9 Hz, 1H), 8.29-8.19 (m, 2H), 8.13 (d, J=1.9 Hz, 1H), 7.89 (dd, J=9.0, 1.9 Hz, 1H), 7.60 (d, J=8.2 Hz, 1H), 4.81-4.74 (m, 1H), 4.64 (s, 2H), 4.46-4.39 (m, 1H), 4.15-4.07 (m, 1H), 3.79-3.71 (m, 1H), 3.64 (t, J=5.9 Hz, 2H), 3.52-3.47 (m, 1H), 3.43 (s, 3H), 2.56-2.52 (m, 2H), 2.44-2.32 (m, 4H), 1.53-1.44 (m, 4H), 1.42-1.31 (m, 2H).

### Example 179

¹H NMR (400 MHz, DMSO-d₆) δ 8.92 (s, 1H), 8.27-8.15 (m, 2H), 8.10 (s, 1H), 7.86 (d, J=9.0 Hz, 1H), 7.57 (d, J=8.1 Hz, 1H), 4.81-4.53 (m, 4H), 4.41 (d, J=9.2 Hz, 1H), 4.15-4.03 (m, 1H), 3.78-3.68 (m, 1H), 3.63 (t, J=5.8 Hz, 2H), 3.50-3.44 (m, 1H), 3.41 (s, 3H), 2.68-2.52 (m, 4H), 2.42-2.27 (m, 2H), 1.92-1.75 (m, 2H), 1.74-1.59 (m, 2H).

### Example 180

¹H NMR (400 MHz, DMSO-d₆) δ 9.03 (d, J=2.9 Hz, 1H), 8.48 (s, 1H), 8.39 (dd, J=9.1, 2.8 Hz, 1H), 8.30 (dd, J=8.0, 2.7 Hz, 1H), 8.06 (d, J=9.0 Hz, 1H), 7.60 (d, J=8.1 Hz, 1H), 5.16-5.03 (m, 1H), 4.74-4.53 (m, 3H), 3.91-3.81 (m, 1H), 3.65 (t, J=5.6 Hz, 2H), 3.57 (s, 3H), 3.18 (s, 3H), 3.02-2.91 (m, 2H), 2.87-2.74 (m, 2H), 2.65-2.53 (m, 4H), 2.43-2.29 (m, 2H), 1.89-1.76 (m, 2H), 1.74-1.59 (m, 2H).

### Example 181

¹H NMR (400 MHz, DMSO-d₆) δ 8.91 (d, J=2.3 Hz, 1H), 8.27-8.15 (m, 2H), 8.09 (d, J=1.8 Hz, 1H), 7.84 (dd, J=9.0, 1.9 Hz, 1H), 7.56 (d, J=8.1 Hz, 1H), 4.76 (t, J=8.4 Hz, 1H), 4.61 (s, 2H), 4.45-4.36 (m, 1H), 4.14-4.03 (m, 1H), 3.76-3.69 (m, 1H), 3.63 (t, J=5.9 Hz, 2H), 3.48-3.45 (m, 1H), 3.40 (s, 3H), 2.72-2.64 (m, 2H), 2.50-2.45 (m, 4H), 1.71-1.59 (m, 4H).

The following compounds were prepared according to the above described methods using different starting materials.

| Ex # | Structure | Name | MS m/z |
|---|---|---|---|
| 182 | 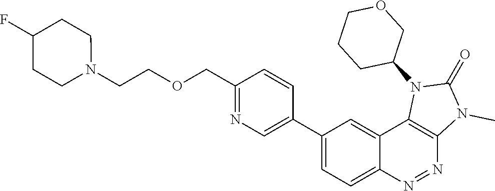 [SMILES: Cn1c(=O)n([C@H]2CCCOC2)c2c3cc(-c4ccc(COCCN5CCC(F)CC5)nc4)ccc3nnc21] | (S)-8-(6-((2-(4- fluoropiperidin-1- yl)ethoxy)methyl)pyridin-3- yl)-3-methyl-1-(tetrahydro- 2H-pyran-3-yl)-1H- imidazo[4,5-c]cinnolin- 2(3H)-one | 521 [M + H]⁺ |
| 183 | 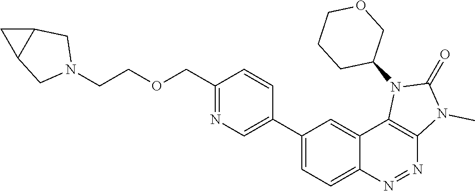 [SMILES: Cn1c(=O)n([C@H]2CCCOC2)c2c3cc(-c4ccc(COCCN5CC6CC6C5)nc4)ccc3nnc21] | 8-(6-((2-(3- azabicyclo[3.1.0]hexan-3- yl)ethoxy)methyl)pyridin-3- yl)-3-methyl-1-((S)- tetrahydro-2H-pyran-3-yl)- 1H-imidazo[4,5-c]cinnolin- 2(3H)-one | 501 [M + H]⁺ |

### Example 182

¹H NMR (400 MHz, CDCl₃) δ 8.92 (s, 1H), 8.56 (d, J=9.0 Hz, 1H), 8.27 (s, 1H), 8.03 (d, J=8.0 Hz, 1H), 7.89 (d, J=8.9 Hz, 1H), 7.65 (d, J=8.0 Hz, 1H), 4.91-4.82 (m, 1H), 4.79-4.60 (m, 3H), 4.48-4.37 (m, 1H), 4.21-4.12 (m, 1H), 4.07-3.98 (m, 1H), 3.80-3.71 (m, 5H), 3.63-3.52 (m, 1H), 2.88-2.75 (m, 1H), 2.75-2.63 (m, 4H), 2.57-2.44 (m, 2H), 2.28-2.15 (m, 1H), 2.03-1.87 (m, 6H).

### Example 183

¹H NMR (400 MHz, CDCl₃) δ 8.89 (s, 1H), 8.53 (d, J=9.0 Hz, 1H), 8.25 (s, 1H), 8.00 (d, J=8.0 Hz, 1H), 7.87 (d, J=9.0 Hz, 1H), 7.63 (d, J=8.1 Hz, 1H), 4.93-4.79 (m, 1H), 4.72 (s, 2H), 4.46-4.36 (m, 1H), 4.18-4.10 (m, 1H), 4.07-3.95 (m, 1H), 3.78-3.64 (m, 5H), 3.60-3.48 (m, 1H), 3.14-3.02 (m, 2H), 2.84-2.70 (m, 3H), 2.50-2.36 (m, 2H), 2.25-2.15 (m, 1H), 1.95-1.86 (m, 2H), 1.40-1.31 (m, 2H), 0.79-0.67 (m, 1H), 0.41-0.30 (m, 1H).

### Example 184 and Example 185

### (S)-1-isopropyl-3-methyl-8-(6-(2,2,2-trifluoro-1-(2-(pyrrolidin-1-yl)ethoxy)ethyl)pyridin-3-yl)-1,3-dihydro-2H-imidazo[4,5-c]cinnolin-2-one and (R)-1-isopropyl-3-methyl-8-(6-(2,2,2-trifluoro-1-(2-(pyrrolidin-1-yl)ethoxy)ethyl)pyridin-3-yl)-1,3-dihydro-2H-imidazo[4,5-c]cinnolin-2-one

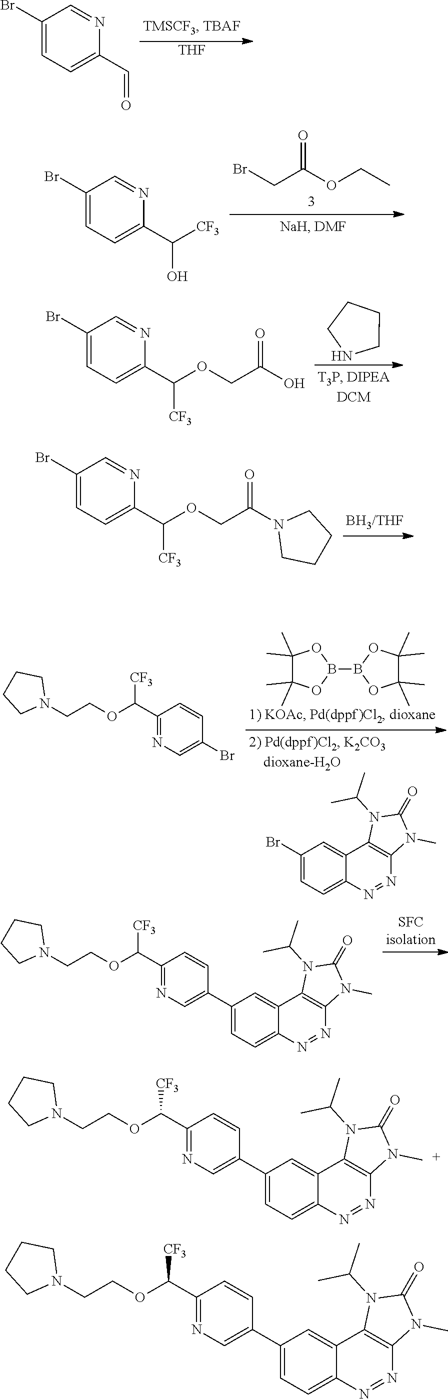

### Step 1: 1-(5-bromopyridin-2-yl)-2,2,2-trifluoroethanol

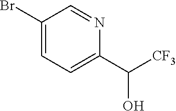 [SMILES: OC(c1ccc(Br)cn1)C(F)(F)F]

To a solution of 5-bromopicolinaldehyde (770 mg, 4.16 mmol) in dry THE (15 mL) was added dropwise TMSCF₃ (885 mg, 6.23 mmol) followed by a solution of TBAF in THF (10.4 mL, 10.4 mmol, 1 M) at 0° C. and the reaction mixture was stirred at room temperature for 16 h. The mixture was poured into water (40 mL) and extracted with EtOAc (20 mL×2). The combined organic layers were washed with brine, dried over anhydrous Na₂SO₄ and filtered, the filtrate was concentrated under reduced pressure. The residue was purified by chromatography on silica gel (PE:EtOAc=25:1 to 10:1) to give the desired product (645 mg, 60.8% yield). LC-MS (ESI) m z: 256 [M+H]⁺.

### Step 2: 2-(1-(5-bromopyridin-2-yl)-2,2,2-trifluoroethoxy)acetic acid

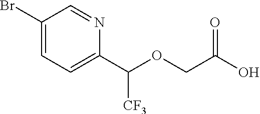 [SMILES: O=C(O)COC(c1ccc(Br)cn1)C(F)(F)F]

To a solution of 1-(5-bromopyridin-2-yl)-2,2,2-trifluoroethanol (455 mg, 1.78 mmol) in dry DMF (5 mL) was added NaH (143 mg, 3.57 mmol) slowly at 0° C. and the reaction mixture was stirred at 0° C. for 30 min. Methyl 2-bromoacetate (0.257 mL, 2.31 mmol) was added dropwise, the resulting reaction mixture was warmed to room temperature slowly and stirred at room temperature for 3 h. The mixture was poured into ice 1 M NaOH solution (15 mL) and stirred at room temperature for 1 h. The mixture was extracted with EtOAc (10 mL×2) and the water layer was adjusted with 1 M HCl solution to pH˜4. It was extracted with EtOAc (20 mL×3), the organic layers were washed with 5% LiCl solution (30 mL) and brine (30 mL), dried over anhydrous Na₂SO₄ and concentrated under reduced pressure to give the crude product (620 mg) as brown oil. LC-MS (ESI) m z: 314 [M+H]⁺.

### Step 3: 2-(1-(5-bromopyridin-2-yl)-2,2,2-trifluoroethoxy)-1-(pyrrolidin-1-yl)ethanone

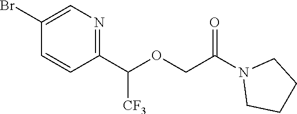 [SMILES: O=C(COC(c1ccc(Br)cn1)C(F)(F)F)N1CCCC1]

A mixture of 2-(1-(5-bromopyridin-2-yl)-2,2,2-trifluoroethoxy)acetic acid (620 mg, 1.98 mmol) and pyrrolidine (155 mg, 2.18 mmol) in DCM (10 mL) was added 50% T₃P in EtOAc (2.52 g, 3.96 mmol) followed by DIEA (1.00 mL, 5.94 mmol) at 0° C. The reaction mixture was stirred at room temperature for 16 h. The mixture was poured into ice sat. NaHCO₃ solution (30 mL) and extracted with DCM (15 mL×2). The organic layer was washed with sat. NH₄Cl solution (30 mL) and brine (30 mL), dried over anhydrous Na₂SO₄ and filtered, the filtrate was concentrated under reduced pressure to give the crude product (820 mg) as brown oil, which was used for next step without further purification. LC-MS (ESI) m z: 367 [M+H]⁺.

### Step 4: 5-bromo-2-(2,2,2-trifluoro-1-(2-(pyrrolidin-1-yl)ethoxy)ethyl)pyridine

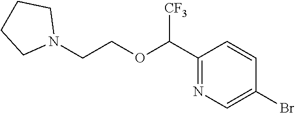 [SMILES: FC(F)(F)C(OCCN1CCCC1)c1ccc(Br)cn1]

To a solution of 2-(1-(5-bromopyridin-2-yl)-2,2,2-trifluoroethoxy)-1-(pyrrolidin-1-yl)ethanone (820 mg, 2.24 mmol) in dry THE (10 mL) was added 1 M BH₃-THF solution (11.2 mL, 11.2 mmol) dropwise at 0° C. and the reaction mixture was stirred at room temperature for 2 h. The mixture was quenched with MeOH (20 mL) and evaporated under reduced pressure. The residue was dissolved in EtOH (20 mL) and stirred at 90° C. for 3 h. The mixture was evaporated under reduced pressure and the residue was purified by chromatography on silica gel (DCM:MeOH=100:1 to 10:1) to give the desired product (380 mg, 60.6% yield for three steps) as light brown oil. LC-MS (ESI) m z: 353 [M+H]⁺.

### Step 5: 1-isopropyl-3-methyl-8-(6-(2,2,2-trifluoro-1-(2-(pyrrolidin-1-yl)ethoxy)ethyl)pyridin-3-yl)-1H-imidazo[4,5-c]cinnolin-2(3H)-one

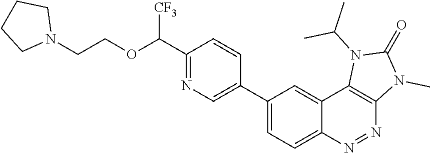 [SMILES: CC(C)n1c(=O)n(C)c2nnc3ccc(-c4ccc(C(OCCN5CCCC5)C(F)(F)F)nc4)cc3c21]

The crude product was prepared in a similar fashion to Example 1, which was purified by chromatography on silica gel (DCM:MeOH=100:1 to 10:1) to give the desired product (160 mg, 60.9% yield for two steps) as an off-white solid. ¹H NMR (400 MHz, CDCl₃) δ 9.00 (d, J=2.0 Hz, 1H), 8.58 (d, J=9.0 Hz, 1H), 8.32 (s, 1H), 8.14 (dd, J=8.1, 2.3 Hz, 1H), 7.89 (dd, J=9.0, 1.7 Hz, 1H), 7.78 (d, J=8.1 Hz, 1H), 5.29-5.17 (m, 1H), 5.06 (q, J=6.4 Hz, 1H), 4.21-4.05 (m, 2H), 3.78 (s, 3H), 3.43-3.14 (m, 6H), 2.12 (s, 4H), 1.81 (dd, J=6.9, 1.0 Hz, 6H). LC-MS (ESI) m z: 515 [M+H]⁺.

### Step 6: (S)-1-isopropyl-3-methyl-8-(6-(2,2,2-trifluoro-1-(2-(pyrrolidin-1-yl)ethoxy)ethyl)pyridin-3-yl)-1,3-dihydro-2H-imidazo[4,5-c]cinnolin-2-one and (R)-1-isopropyl-3-methyl-8-(6-(2,2,2-trifluoro-1-(2-(pyrrolidin-1-yl)ethoxy)ethyl)pyridin-3-yl)-1,3-dihydro-2H-imidazo[4,5-c]cinnolin-2-one

1-isopropyl-3-methyl-8-(6-(2,2,2-trifluoro-1-(2-(pyrrolidin-1-yl)ethoxy)ethyl)pyridin-3-yl)-1H-imidazo[4,5-c]cinnolin-2(3H)-one was subsequently separated by chiral SFC to give two isomers.

(S)-1-isopropyl-3-methyl-8-(6-(2,2,2-trifluoro-1-(2-(pyrrolidin-1-yl)ethoxy)ethyl)pyridin-3-yl)-1,3-dihydro-2H-imidazo[4,5-c]cinnolin-2-one as white solid.

¹H NMR (400 MHz, CDCl₃) δ 8.96 (s, 1H), 8.57 (d, J=9.0 Hz, 1H), 8.29 (s, 1H), 8.09 (dd, J=8.1, 2.1 Hz, 1H), 7.87 (d, J=9.0 Hz, 1H), 7.79 (d, J=8.1 Hz, 1H), 5.27-5.16 (m, 1H), 4.97 (q, J=6.5 Hz, 1H), 3.86 (ddt, J=13.1, 9.9, 5.1 Hz, 2H), 3.77 (s, 3H), 2.89 (h, J=7.0 Hz, 2H), 2.72 (s, 4H), 1.84 (s, 4H), 1.79 (d, J=6.9 Hz, 6H).

(R)-1-isopropyl-3-methyl-8-(6-(2,2,2-trifluoro-1-(2-(pyrrolidin-1-yl)ethoxy)ethyl)pyridin-3-yl)-1,3-dihydro-2H-imidazo[4,5-c]cinnolin-2-one as white solid.

¹H NMR (400 MHz, CDCl₃) δ 8.99 (d, J=2.1 Hz, 1H), 8.56 (d, J=9.0 Hz, 1H), 8.30 (s, 1H), 8.12 (dd, J=8.1, 2.2 Hz, 1H), 7.87 (d, J=9.1 Hz, 1H), 7.77 (d, J=8.1 Hz, 1H), 5.27-5.15 (m, 1H), 5.05 (q, J=6.3 Hz, 1H), 4.14 (ddd, J=34.3, 11.1, 6.0 Hz, 2H), 3.77 (s, 3H), 3.32 (d, J=4.5 Hz, 6H), 2.12 (s, 4H), 1.79 (d, J=6.9 Hz, 6H).

SFC condition: Instrument: MG II preparative SFC(SFC-14) Column: Cellulose-2, 250×30 mm I.D., 5 m Mobile phase: A for CO₂ and B for Isopropanol (0.1% NH₃H₂O)

Gradient: B 40% Flow rate: 60 mL/min Back pressure: 100 bar Column temperature: 38° C. Wavelength: 220 nm Cycle time: ˜22 min

The following compounds were prepared according to the above described methods using different starting materials.

| | | | MS |
|---|---|---|---|
| Ex # | Structure | Name | m/z |
| 186 | 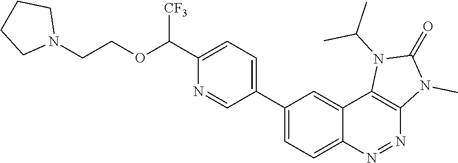 [SMILES: CC(C)n1c(=O)n(C)c2nnc3ccc(-c4ccc(C(OCCN5CCCC5)C(F)(F)F)nc4)cc3c21] | 1-isopropyl-3-methyl-8-(6- (2,2,2-trifluoro-1-(2- (pyrrolidin-1- yl)ethoxy)ethyl)pyridin-3- yl)-1,3-dihydro-2H- imidazo[4,5-c]cinnolin-2- one | 515 [M + H]⁺ |
| 187 | 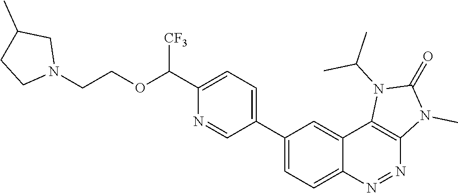 [SMILES: CC1CCN(CCOC(c2ccc(-c3ccc4nnc5c(c4c3)n(C(C)C)c(=O)n5C)cn2)C(F)(F)F)C1] | 1-isopropyl-3-methyl-8-(6- (2,2,2-trifluoro-1-(2-(3- methylpyrrolidin-1- yl)ethoxy)ethyl)pyridin-3- yl)-1,3-dihydro-2H- imidazo[4,5-c]cinnolin-2- one | 529 [M + H]⁺ |
| 188 | 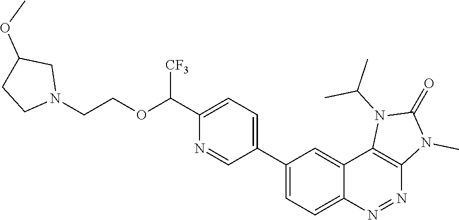 [SMILES: COC1CCN(CCOC(c2ccc(-c3ccc4nnc5c(c4c3)n(C(C)C)c(=O)n5C)cn2)C(F)(F)F)C1] | 1-isopropyl-3-methyl-8-(6- (2,2,2-trifluoro-1-(2-(3- methoxypyrrolidin-1- yl)ethoxy)ethyl)pyridin-3- yl)-1,3-dihydro-2H- imidazo[4,5-c]cinnolin-2- one | 545 [M + H]⁺ |
| 189 | 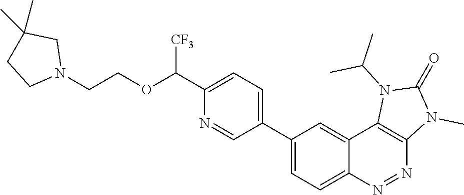 | 8-(6-(1-(2-(3,3- dimethylpyrrolidin-1- yl)ethoxy)-2,2,2- trifluoroethyl)pyridin-3-yl)- 1-isopropyl-3-methyl-1,3- dihydro-2H-imidazo[4,5- c]cinnolin-2-one | 543 [M + H]⁺ |
| 190 | 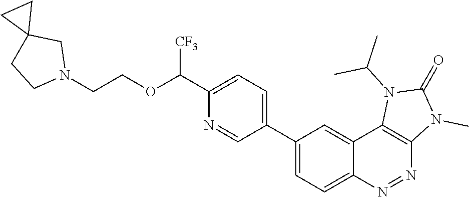 [SMILES: CC(C)n1c(=O)n(C)c2nnc3ccc(-c4ccc(C(OCCN5CCC6(CC6)C5)C(F)(F)F)nc4)cc3c21] | 8-(6-(1-(2-(5- azaspiro[2.4]heptan-5- yl)ethoxy)-2,2,2- trifluoroethyl)pyridin-3-yl)- 1-isopropyl-3-methyl-1,3- dihydro-2H-imidazo[4,5- c]cinnolin-2-one | 541 [M + H]⁺ |
| 191 | 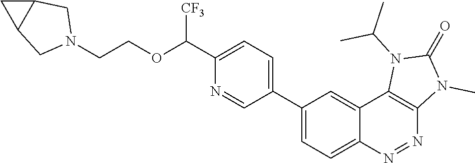 [SMILES: CC(C)n1c(=O)n(C)c2nnc3ccc(-c4ccc(C(OCCN5CC6CC6C5)C(F)(F)F)nc4)cc3c21] | 8-(6-(1-(2-(3- azabicyclo[3.1.0]hexan-3- yl)ethoxy)-2,2,2- trifluoroethyl)pyridin-3-yl)- 1-isopropyl-3-methyl-1,3- dihydro-2H-imidazo[4,5- c]cinnolin-2-one | 527 [M + H]⁺ |
| 192 | 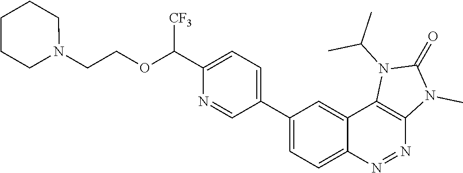 [SMILES: CC(C)n1c(=O)n(C)c2nnc3ccc(-c4ccc(C(OCCN5CCCCC5)C(F)(F)F)nc4)cc3c21] | 1-isopropyl-3-methyl-8-(6- (2,2,2-trifluoro-1-(2- (piperidin-1- yl)ethoxy)ethyl)pyridin-3- yl)-1,3-dihydro-2H- imidazo[4,5-c]cinnolin-2- one | 529 [M + H]⁺ |
| 193 | 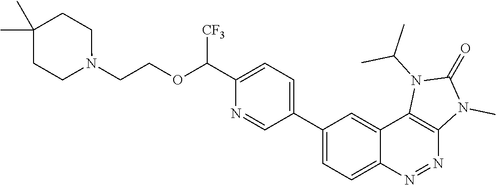 | 8-(6-(1-(2-(4,4- dimethylpiperidin-1- yl)ethoxy)-2,2,2- trifluoroethyl)pyridin-3-yl)- 1-isopropyl-3-methyl-1,3- dihydro-2H-imidazo[4,5- c]cinnolin-2-one | 557 [M + H]⁺ |
| 194 | 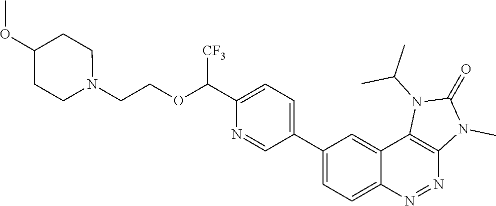 [SMILES: COC1CCN(CCOC(c2ccc(-c3ccc4nnc5c(c4c3)n(C(C)C)c(=O)n5C)cn2)C(F)(F)F)CC1] | 1-isopropyl-3-methyl-8-(6- (2,2,2-trifluoro-1-(2-(4- methoxypiperidin-1- yl)ethoxy)ethyl)pyridin-3- yl)-1,3-dihydro-2H- imidazo[4,5-c]cinnolin-2- one | 559 [M + H]⁺ |
| 195 | 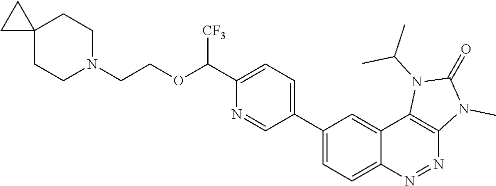 [SMILES: CC(C)n1c(=O)n(C)c2nnc3ccc(-c4ccc(C(OCCN5CCC6(CC5)CC6)C(F)(F)F)nc4)cc3c21] | 8-(6-(1-(2-(6- azaspiro[2.5]octan-6- yl)ethoxy)-2,2,2- trifluoroethyl)pyridin-3-yl)- 1-isopropyl-3-methyl-1,3- dihydro-2H-imidazo[4,5- c]cinnolin-2-one | 555 [M + H]⁺ |
| 196 | 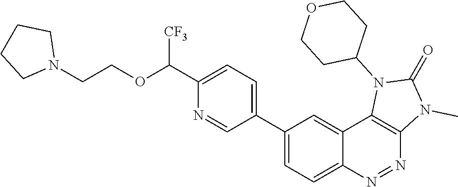 [SMILES: Cn1c(=O)n(C2CCOCC2)c2c3cc(-c4ccc(C(OCCN5CCCC5)C(F)(F)F)nc4)ccc3nnc21] | 3-methyl-1 -(tetrahydro-2H- pyran-4-yl)-8-(6-(2,2,2- trifluoro-1-(2-(pyrrolidin-1- yl)ethoxy)ethyl)pyridin-3- yl)-1,3-dihydro-2H- imidazo[4,5-c]cinnolin-2- one | 557 [M + H]⁺ |
| 197 | 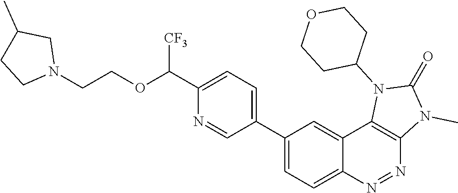 [SMILES: CC1CCN(CCOC(c2ccc(-c3ccc4nnc5c(c4c3)n(C3CCOCC3)c(=O)n5C)cn2)C(F)(F)F)C1] | 3-methyl-1-(tetrahydro-2H- pyran-4-yl)-8-(6-(2,2,2- trifluoro-1-(2-(3- methylpyrrolidin-1- yl)ethoxy)ethyl)pyridin-3- yl)-1,3-dihydro-2H- imidazo[4,5-c]cinnolin-2- one | 571 [M + H]⁺ |
| 198 | 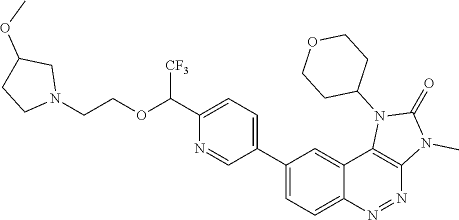 [SMILES: COC1CCN(CCOC(c2ccc(-c3ccc4nnc5c(c4c3)n(C3CCOCC3)c(=O)n5C)cn2)C(F)(F)F)C1] | 3-methyl-1-(tetrahydro-2H- pyran-4-yl)-8-(6-(2,2,2- trifluoro-1-(2-(3- methoxypyrrolidin-1- yl)ethoxy)ethyl)pyridin-3- yl)-1,3-dihydro-2H- imidazo[4,5-c]cinnolin-2- one | 587 [M + H]⁺ |
| 199 | 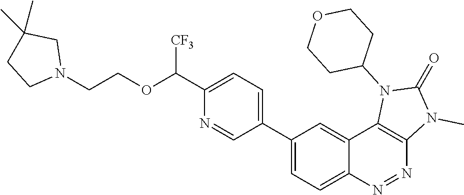 | 8-(6-(1-(2-(3,3- dimethylpyrrolidin-1- yl)ethoxy)-2,2,2- trifluoroethyl)pyridin-3-yl)- 3-methyl-1 -(tetrahydro-2H- pyran-4-yl)-1,3-dihydro-2H- imidazo[4,5-c]cinnolin-2- one | 585 [M + H]⁺ |
| 200 | 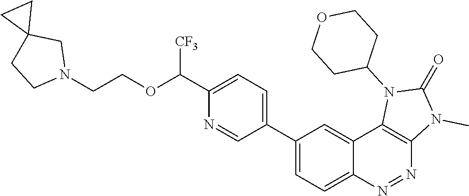 [SMILES: Cn1c(=O)n(C2CCOCC2)c2c3cc(-c4ccc(C(OCCN5CCC6(CC6)C5)C(F)(F)F)nc4)ccc3nnc21] | 8-(6-(1-(2-(5- azaspiro[2.4]heptan-5- yl)ethoxy)-2,2,2- trifluoroethyl)pyridin-3-yl)- 3-methyl-1-(tetrahydro-2H- pyran-4-yl)-1,3-dihydro-2H- imidazo[4,5-c]cinnolin-2- one | 583 [M + H]⁺ |
| 201 | 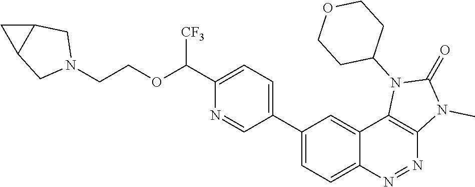 [SMILES: Cn1c(=O)n(C2CCOCC2)c2c3cc(-c4ccc(C(OCCN5CC6CC6C5)C(F)(F)F)nc4)ccc3nnc21] | 8-(6-(1-(2-(3- azabicyclo[3.1.0]hexan-3- yl)ethoxy)-2,2,2- trifluoroethyl)pyridin-3-yl)- 3-methyl-1 -(tetrahydro-2H- pyran-4-yl)-1,3-dihydro-2H- imidazo[4,5-c]cinnolin-2- one | 569 [M + H]⁺ |
| 202 | 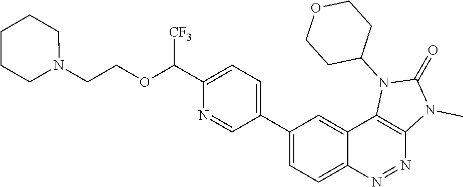 [SMILES: Cn1c(=O)n(C2CCOCC2)c2c3cc(-c4ccc(C(OCCN5CCCCC5)C(F)(F)F)nc4)ccc3nnc21] | 3-methyl-1 -(tetrahydro-2H- pyran-4-yl)-8-(6-(2,2,2- trifluoro-1-(2-(piperidin-1- yl)ethoxy)ethyl)pyridin-3- yl)-1,3-dihydro-2H- imidazo[4,5-c]cinnolin-2- one | 571 [M + H]⁺ |
| 203 | 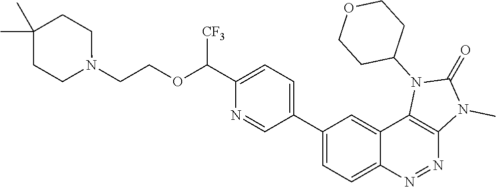 | 8-(6-(1-(2-(4,4- dimethylpiperidin-1- yl)ethoxy)-2,2,2- trifluoroethyl)pyridin-3-yl)- 3-methyl-1-(tetrahydro-2H- pyran-4-yl)-1,3-dihydro-2H- imidazo[4,5-c]cinnolin-2- one | 599 [M + H]⁺ |
| 204 | 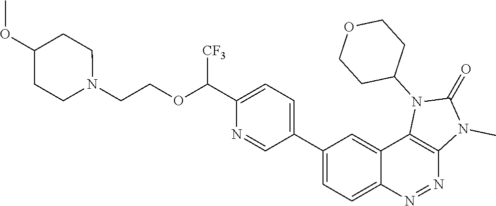 [SMILES: COC1CCN(CCOC(c2ccc(-c3ccc4nnc5c(c4c3)n(C3CCOCC3)c(=O)n5C)cn2)C(F)(F)F)CC1] | 3-methyl-1-(tetrahydro-2H- pyran-4-yl)-8-(6-(2,2,2- trifluoro-1-(2-(4- methoxypiperidin-1- yl)ethoxy)ethyl)pyridin-3- yl)-1,3-dihydro-2H- imidazo[4,5-c]cinnolin-2- one | 601 [M + H]⁺ |
| 205 | 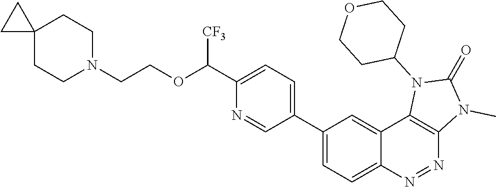 [SMILES: Cn1c(=O)n(C2CCOCC2)c2c3cc(-c4ccc(C(OCCN5CCC6(CC5)CC6)C(F)(F)F)nc4)ccc3nnc21] | 8-(6-(1-(2-(6- azaspiro[2.5]octan-6- yl)ethoxy)-2,2,2- trifluoroethyl)pyridin-3-yl)- 3-methyl-1 -(tetrahydro-2H- pyran-4-yl)-1,3-dihydro-2H- imidazo[4,5-c]cinnolin-2- one | 597 [M + H]⁺ |
| 206 | 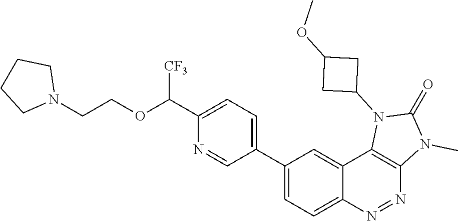 [SMILES: COC1CC(n2c(=O)n(C)c3nnc4ccc(-c5ccc(C(OCCN6CCCC6)C(F)(F)F)nc5)cc4c32)C1] | 1-(3-methoxycyclobutyl)-3- methyl-8-(6-(2,2,2-trifluoro- 1-(2-(pyrrolidin-1- yl)ethoxy)ethyl)pyridin-3- yl)-1,3-dihydro-2H- imidazo[4,5-c]cinnolin-2- one | 557 [M + H]⁺ |
| 207 | 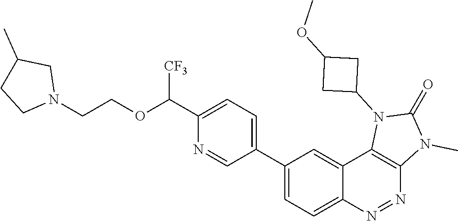 [SMILES: COC1CC(n2c(=O)n(C)c3nnc4ccc(-c5ccc(C(OCCN6CCC(C)C6)C(F)(F)F)nc5)cc4c32)C1] | 1-(3-methoxycyclobutyl)-3- methyl-8-(6-(2,2,2-trifluoro- 1-(2-(3-methylpyrrolidin-1- yl)ethoxy)ethyl)pyridin-3- yl)-1,3-dihydro-2H- imidazo[4,5-c]cinnolin-2- one | 571 [M + H]⁺ |
| 208 | 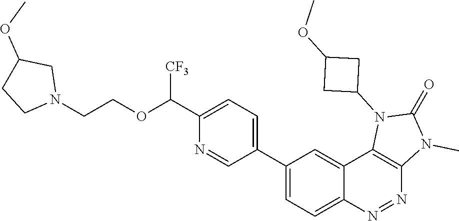 [SMILES: COC1CC(n2c(=O)n(C)c3nnc4ccc(-c5ccc(C(OCCN6CCC(OC)C6)C(F)(F)F)nc5)cc4c32)C1] | 1-(3-methoxycyclobutyl)-3- methyl-8-(6-(2,2,2-trifluoro- 1-(2-(3-methoxypyrrolidin- 1-yl)ethoxy)ethyl)pyridin-3- yl)-1,3-dihydro-2H- imidazo[4,5-c]cinnolin-2- one | 587 [M + H]⁺ |
| 209 | 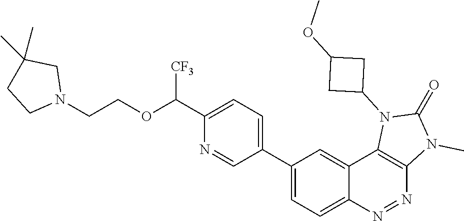 | 8-(6-(1-(2-(3,3- dimethylpyrrolidin-1- yl)ethoxy)-2,2,2- trifluoroethyl)pyridin-3-yl)- 1-(3-methoxycyclobutyl)-3- methyl-1,3-dihydro-2H- imidazo[4,5-c]cinnolin-2- one | 585 [M + H]⁺ |
| 210 | 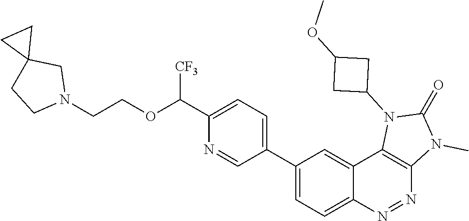 [SMILES: COC1CC(n2c(=O)n(C)c3nnc4ccc(-c5ccc(C(OCCN6CCC7(CC7)C6)C(F)(F)F)nc5)cc4c32)C1] | 8-(6-(1-(2-(5- azaspiro[2.4]heptan-5- yl)ethoxy)-2,2,2- trifluoroethyl)pyridin-3-yl)- 1-(3-methoxycyclobutyl)-3- methyl-1,3-dihydro-2H- imidazo[4,5-c]cinnolin-2- one | 583 [M + H]⁺ |
| 211 | 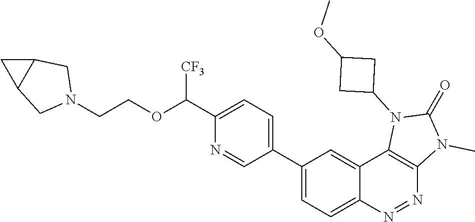 [SMILES: COC1CC(n2c(=O)n(C)c3nnc4ccc(-c5ccc(C(OCCN6CC7CC7C6)C(F)(F)F)nc5)cc4c32)C1] | 8-(6-(1-(2-(3- azabicyclo[3.1.0]hexan-3- yl)ethoxy)-2,2,2- trifluoroethyl)pyridin-3-yl)- 1-(3-methoxycyclobutyl)-3- methyl-1,3-dihydro-2H- imidazo[4,5-c]cinnolin-2- one | 569 [M + H]⁺ |
| 212 | 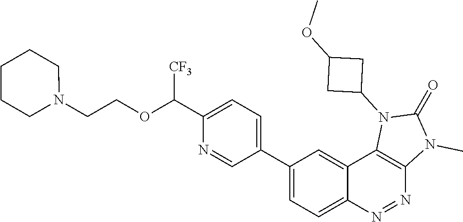 [SMILES: COC1CC(n2c(=O)n(C)c3nnc4ccc(-c5ccc(C(OCCN6CCCCC6)C(F)(F)F)nc5)cc4c32)C1] | 1-(3-methoxycyclobutyl)-3- methyl-8-(6-(2,2,2-trifluoro- 1-(2-(piperidin-1- yl)ethoxy)ethyl)pyridin-3- yl)-1,3-dihydro-2H- imidazo[4,5-c]cinnolin-2- one | 571 [M + H]⁺ |
| 213 | 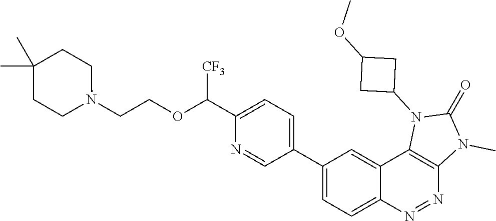 | 8-(6-(1-(2-(4,4- dimethylpiperidin-1- yl)ethoxy)-2,2,2- trifluoroethyl)pyridin-3-yl)- 1-(3-methoxycyclobutyl)-3- methyl-1,3-dihydro-2H- imidazo[4,5-c]cinnolin-2- one | 599 [M + H]⁺ |
| 214 | 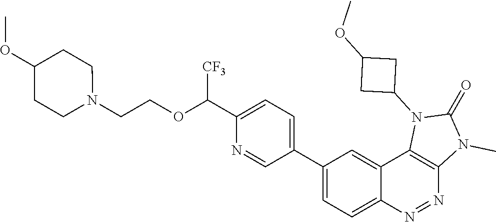 [SMILES: COC1CCN(CCOC(c2ccc(-c3ccc4nnc5c(c4c3)n(C3CC(OC)C3)c(=O)n5C)cn2)C(F)(F)F)CC1] | 1-(3-methoxycyclobutyl)-3- methyl-8-(6-(2,2,2-trifluoro- 1-(2-(4-methoxypiperidin-1- yl)ethoxy)ethyl)pyridin-3- yl)-1,3-dihydro-2H- imidazo[4,5-c]cinnolin-2- one | 601 [M + H]⁺ |
| 215 | 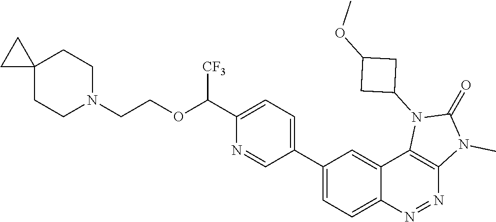 [SMILES: COC1CC(n2c(=O)n(C)c3nnc4ccc(-c5ccc(C(OCCN6CCC7(CC6)CC7)C(F)(F)F)nc5)cc4c32)C1] | 8-(6-(1-(2-(6- azaspiro[2.5]octan-6- yl)ethoxy)-2,2,2- trifluoroethyl)pyridin-3-yl)- 1-(3-methoxycyclobutyl)-3- methyl-1,3-dihydro-2H- imidazo[4,5-c]cinnolin-2- one | 597 [M + H]⁺ |

### Example 216

### (R)-2-(1-(5-(1-isopropyl-3-methyl-2-oxo-2,3-dihydro-1H-imidazo[4,5-c]cinnolin-8-yl)pyridin-2-yl)ethoxy)acetic acid

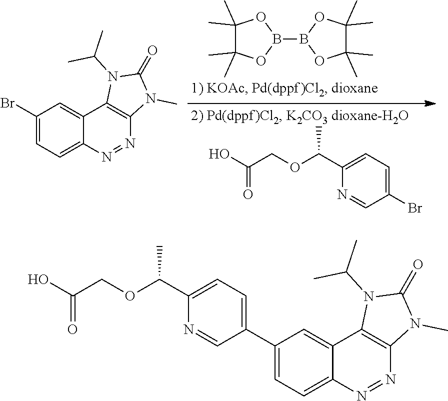

### Step 1: (R)-2-(1-(5-(1-isopropyl-3-methyl-2-oxo-2,3-dihydro-1H-imidazo[4,5-c]cinnolin-8-yl)pyridin-2-yl)ethoxy)acetic acid

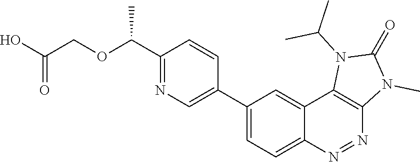 [SMILES: CC(C)n1c(=O)n(C)c2nnc3ccc(-c4ccc([C@@H](C)OCC(=O)O)nc4)cc3c21]

The crude product was prepared in a similar fashion to Example 1, which was purified by prep-TLC (DCM:MeOH=10:1) to give the crude product and it was purified by prep-HPLC to give the desired product (100 mg, 38.0% yield for two steps) as yellow syrupy. ¹H NMR (400 MHz, DMSO-d₆) δ 9.09 (s, 1H), 8.56 (s, 1H), 8.46-8.38 (m, 2H), 8.24 (d, J=9.1 Hz, 1H), 7.73 (d, J=8.2 Hz, 1H), 5.45-5.35 (m, 1H), 4.79-4.71 (m, 1H), 4.06 (q, J=38.5, 16.6 Hz, 2H), 3.59 (s, 3H), 1.68 (d, J=6.7 Hz, 6H), 1.50 (d, J=6.6 Hz, 3H). LC-MS (ESI) m z: 422 [M+H]⁺.

### Example 217

### 8-(6-(2-(2-(3-azabicyclo[3.1.0]hexan-3-yl)ethoxy)propan-2-yl)pyridin-3-yl)-1-isopropyl-3-methyl-1H-imidazo[4,5-c]cinnolin-2(3H)-one

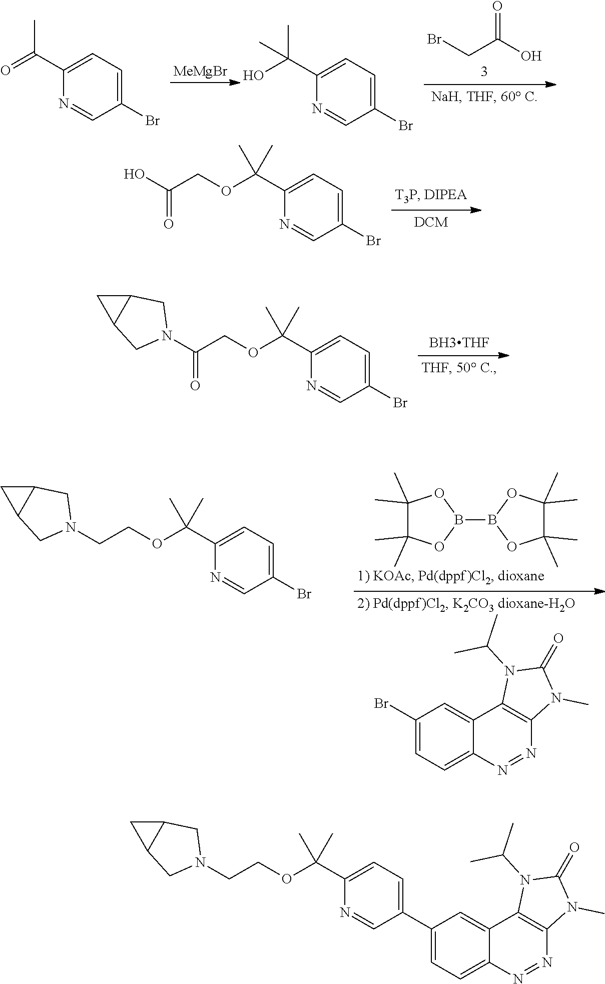

### Step 1: 2-(5-bromopyridin-2-yl)propan-2-ol

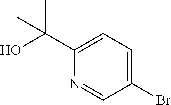

To a solution of 1-(5-bromopyridin-2-yl)ethanone (3.00 g, 15.1 mmol) in dry THF (30 mL) was added MeMgBr (3 M) in THF (7.54 mL, 22.6 mmol) dropwise at 0° C. and the reaction mixture was stirred at room temperature for 16 h. The mixture was poured into ice sat. NH₄Cl solution (50 mL) and extracted with EtOAc (30 mL×2). The combined organic layers were washed with brine (50 mL), dried over anhydrous Na₂SO₄ and concentrated under reduced pressure. The residue was purified by chromatography on silica gel (PE:EtOAc=25:1 to 5:1) to give the desired product (1.60 g, 49.3%) as an off-white solid. LC-MS (ESI) m z: 216 [M+H]⁺.

### Step 2: 2-((2-(5-bromopyridin-2-yl)propan-2-yl)oxy)acetic acid

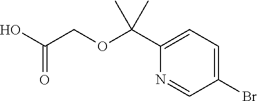

To a solution of 2-(5-bromopyridin-2-yl)propan-2-ol (1.00 g, 4.65 mmol) in dry THE (10 mL) was added NaH (372 mg, 9.30 mmol) slowly at 0° C. and the reaction mixture was stirred at 0° C. for 30 min. To the mixture was added 2-bromoacetic acid (834 mg, 6.05 mmol) dropwise, the resulting reaction mixture was warmed to room temperature slowly and stirred at room temperature for 2 h. The mixture was poured into ice water (10 mL) and adjusted with 1 M HCl solution until pH˜4, extracted with EtOAc (15 mL×3). The organic layers were dried over anhydrous Na₂SO₄ and concentrated under reduced pressure to give the crude product (1.00 g) as brown oil. LC-MS (ESI) m z: 274 [M+H]⁺.

### Step 3: 1-(3-azabicyclo[3.1.0]hexan-3-yl)-2-((2-(5-bromopyridin-2-yl)propan-2-yl)oxy)ethanone

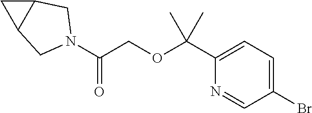 [SMILES: CC(C)(OCC(=O)N1CC2CC2C1)c1ccc(Br)cn1]

A mixture of 1-((5-bromopyridin-2-yl)methoxy)cyclopropanecarboxylic acid (1.00 g, 3.66 mmol) and 3-azabicyclo[3.1.0]hexane (334 mg, 4.03 mmol) in DCM (10 mL) was added 50% T₃P in EtOAc (4.66 g, 7.32 mmol) followed by DIEA (1.80 mL, 11.0 mmol) at 0° C. And the reaction mixture was stirred at room temperature for 16 h. The mixture was poured into ice sat. NaHCO₃ solution (50 mL) and extracted with DCM (20 mL×2). The organic layer was washed with sat. NH₄Cl solution (50 mL) and brine (50 mL), dried over anhydrous Na₂SO₄ and concentrated under reduced pressure to give the crude product (980 mg) as brown oil. LC-MS (ESI) m z: 339 [M+H]⁺.

### Step 4: 3-(2-((2-(5-bromopyridin-2-yl)propan-2-yl)oxy)ethyl)-3-azabicyclo[3.1.0]hexane

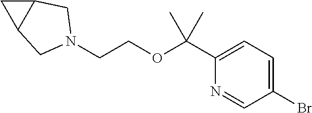 [SMILES: CC(C)(OCCN1CC2CC2C1)c1ccc(Br)cn1]

To a solution of 1-(3-azabicyclo[3.1.0]hexan-3-yl)-2-((2-(5-bromopyridin-2-yl)propan-2-yl)oxy)ethanone (980 mg, 2.90 mmol) in dry THE (10 mL) was added 1 M BH₃-THF solution (14.5 mL, 14.5 mmol) dropwise at 0° C. and the reaction mixture was stirred at room temperature for 2 h. The mixture was quenched with MeOH (20 mL) and evaporated under reduced pressure. The residue was dissolved in EtOH (30 mL) and stirred at 90° C. for 3 h. The mixture was evaporated under reduced pressure and the residue was purified by chromatography on silica gel (DCM:MeOH=100:1 to 10:1) to give the desired product (450 mg, 29.9% yield for three steps) as light brown oil. LC-MS (ESI) m z: 325 [M+H]⁺.

### Step 5: 8-(6-(2-(2-(3-azabicyclo[3.1.0]hexan-3-yl)ethoxy)propan-2-yl)pyridin-3-yl)-1-isopropyl-3-methyl-1H-imidazo[4,5-c]cinnolin-2(3H)-one

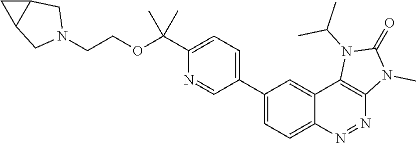 [SMILES: CC(C)n1c(=O)n(C)c2nnc3ccc(-c4ccc(C(C)(C)OCCN5CC6CC6C5)nc4)cc3c21]

The crude product was prepared in a similar fashion to Example 1, which was purified by chromatography on silica gel (DCM:MeOH=100:1 to 10:1) to give the desired product (60.0 mg, 20.0% yield) as an off-white solid. ¹H NMR (400 MHz, DMSO-d₆) δ 9.05 (s, 1H), 8.50-8.36 (m, 2H), 8.27 (d, J=8.0 Hz, 1H), 8.08 (d, J=9.1 Hz, 1H), 7.74 (d, J=8.3 Hz, 1H), 5.38-5.26 (m, 1H), 3.59 (s, 3H), 3.40-3.34 (m, 2H), 2.93 (d, J=8.6 Hz, 2H), 2.60 (t, J=6.2 Hz, 2H), 2.28 (d, J=8.7 Hz, 2H), 1.66 (d, J=6.7 Hz, 6H), 1.51 (s, 6H), 1.35-1.24 (m, 2H), 0.64-0.53 (m, 1H), 0.33-0.23 (m, 1H). LC-MS (ESI) m z: 487 [M+H]⁺.

### Example 218

### 8-(6-((2-(3-azabicyclo[3.1.0]hexan-3-yl)-1,1-difluoroethoxy)methyl)pyridin-3-yl)-1-isopropyl-3-methyl-1H-imidazo[4,5-c]cinnolin-2(3H)-one

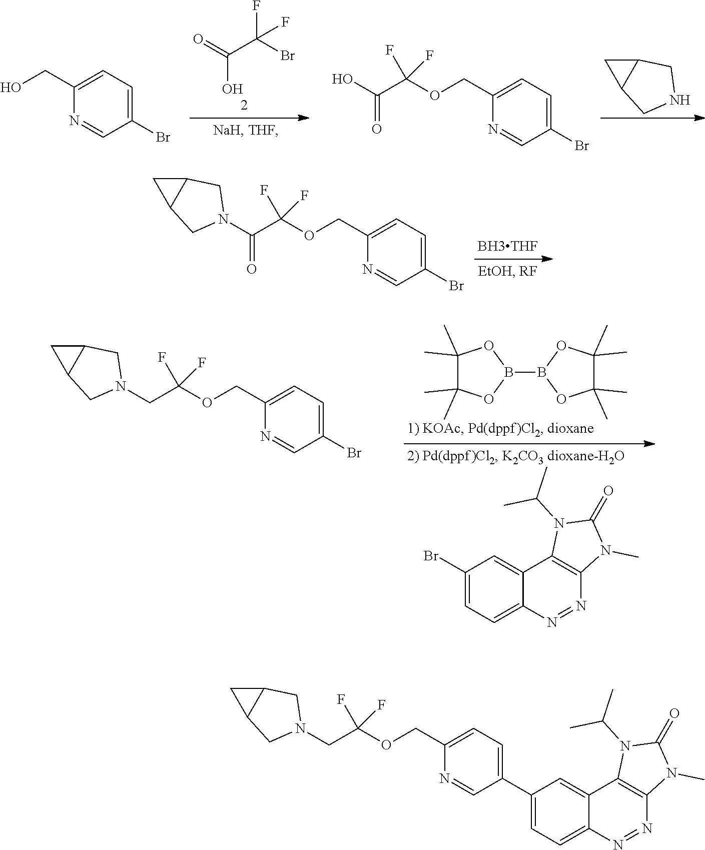

### Step 1: 2-((5-bromopyridin-2-yl)methoxy)-2,2-difluoroacetic acid

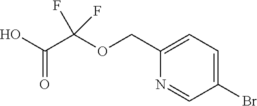 [SMILES: O=C(O)C(F)(F)OCc1ccc(Br)cn1]

To a solution of 2-(5-bromopyridin-2-yl)propan-2-ol (2.00 g, 10.7 mmol) in dry THF (20 mL) was added NaH (856 mg, 21.4 mmol) slowly at 0° C. and the reaction mixture was stirred at 0° C. for 30 min. Then to the mixture was added 2-bromo-2,2-difluoroacetic acid (2.23 g, 12.8 mmol) dropwise, the resulting reaction mixture was warmed to room temperature slowly and stirred at room temperature for 2 h. The mixture was poured into ice water (10 mL) and adjusted with 1 M HCl solution until pH˜4, extracted with EtOAc (15 mL×3). The organic layers were dried over anhydrous Na₂SO₄ and concentrated under reduced pressure to give the crude product (680 mg) as brown oil. LC-MS (ESI) m z: 282 [M+H]⁺.

### Step 2: 1-(3-azabicyclo[3.1.0]hexan-3-yl)-2-((5-bromopyridin-2-yl)methoxy)-2,2-difluoroethanone

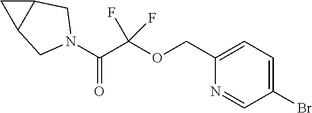 [SMILES: O=C(N1CC2CC2C1)C(F)(F)OCc1ccc(Br)cn1]

To a mixture of 2-((5-bromopyridin-2-yl)methoxy)-2,2-difluoroacetic acid (680 mg, 2.42 mmol) and 3-azabicyclo[3.1.0]hexane (201 mg, 2.42 mmol) in DCM (10 mL) was added 50% T₃P in EtOAc (3.08 g, 4.84 mmol) followed by DIEA (937 mg, 7.26 mmol) at 0° C. And the reaction mixture was stirred at room temperature for 16 h. The mixture was poured into ice sat. NaHCO₃ solution (50 mL) and extracted with DCM (20 mL×2). The organic layer was washed with sat. NH₄Cl solution (50 mL) and brine (50 mL), dried over anhydrous Na₂SO₄ and filtered, the filtrate was concentrated under reduced pressure to give the crude product (730 mg) as brown oil. LC-MS (ESI) m z: 347 [M+H]⁺.

### Step 3: 3-(2-((5-bromopyridin-2-yl)methoxy)-2,2-difluoroethyl)-3-azabicyclo[3.1.0]hexane

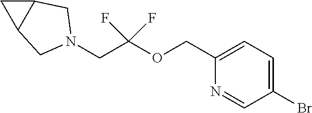 [SMILES: FC(F)(CN1CC2CC2C1)OCc1ccc(Br)cn1]

To a solution of 1-(3-azabicyclo[3.1.0]hexan-3-yl)-2-((5-bromopyridin-2-yl)methoxy)-2,2-difluoroethanone (730 mg, 2.11 mmol) in dry THF (10 mL) was added 1 M BH₃-THF solution (10.6 mL, 10.6 mmol) dropwise at 0° C. and the reaction mixture was stirred at room temperature for 2 h. The mixture was quenched with MeOH (20 mL) and evaporated under reduced pressure. The residue was dissolved in EtOH (30 mL) and stirred at 90° C. for 2 h. The mixture was evaporated under reduced pressure and the residue was purified by chromatography on silica gel (DCM:MeOH=100:1 to 20:1) to give the desired product (470 mg, 13.2% yield for three steps) as light brown oil. LC-MS (ESI) m z: 333 [M+H]⁺.

### Step 4: 8-(6-((2-(3-azabicyclo[3.1.0]hexan-3-yl)-1,1-difluoroethoxy)methyl)pyridin-3-yl)-1-isopropyl-3-methyl-1H-imidazo[4,5-c]cinnolin-2(3H)-one

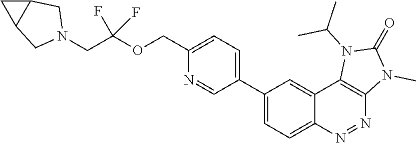 [SMILES: CC(C)n1c(=O)n(C)c2nnc3ccc(-c4ccc(COC(F)(F)CN5CC6CC6C5)nc4)cc3c21]

The crude product was prepared in a similar fashion to Example 1, which was purified by chromatography on silica gel (DCM:MeOH=100:1 to 10:1) to give the desired product (30.0 mg, 10.1% yield in two steps). ¹H NMR (400 MHz, dmso) δ 9.08 (s, 1H), 8.51-8.39 (m, 2H), 8.35 (d, J=8.0 Hz, 1H), 8.10 (d, J=9.1 Hz, 1H), 7.62 (d, J=8.1 Hz, 1H), 5.37-5.25 (m, 1H), 5.05 (s, 2H), 3.59 (s, 3H), 3.12-2.93 (m, 4H), 2.58 (d, J=8.6 Hz, 2H), 1.66 (d, J=6.7 Hz, 6H), 1.40-1.31 (m, 2H), 0.57-0.49 (m, 1H), 0.37-0.28 (m, 1H). LC-MS (ESI) m z: 495 [M+H]⁺.

### Example 219

### 1-isopropyl-3-methyl-8-(6-(1-(2-(pyrrolidin-1-yl)ethoxy)propyl)pyridin-3-yl)-1H-imidazo[4,5-c]cinnolin-2(3H)-one

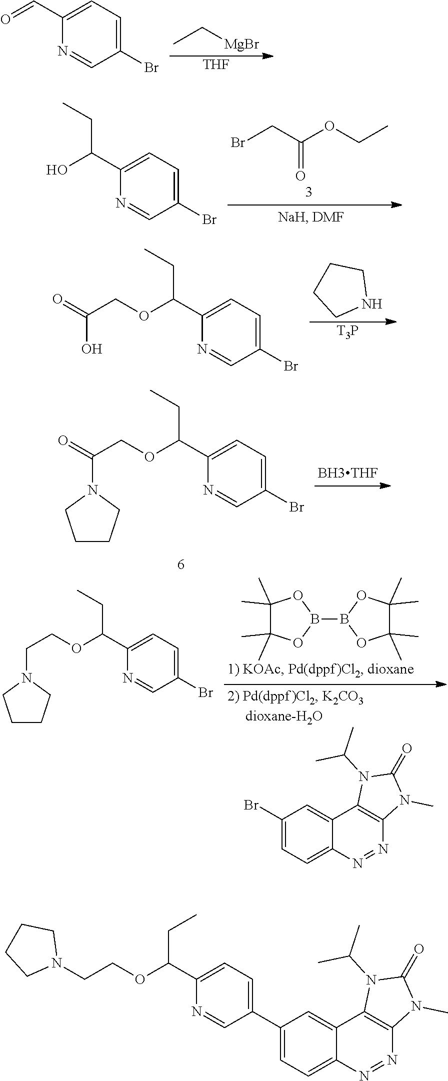

### Step 1: 1-(5-bromopyridin-2-yl)propan-1-ol

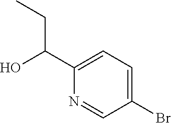

To a solution of 5-bromopicolinaldehyde (1.00 g, 5.41 mmol) in dry THF (10 mL) was added Ethylmagnesium bromide (1 M) in THF (8.12 mL, 8.12 mmol) dropwise at 0° C. and the reaction mixture was stirred at room temperature for 16 h. The mixture was poured into ice sat. NH₄Cl solution (50 mL) and extracted with EtOAc (30 mL×2). The combined organic layers were washed with brine (50 mL), dried over anhydrous Na₂SO₄ and filtered, the filtrate was concentrated under reduced pressure to give the crude product (1.20 g) as brown oil. LC-MS (ESI) m z: 216 [M+H]⁺.

### Step 2: 2-(1-(5-bromopyridin-2-yl)propoxy)acetic acid

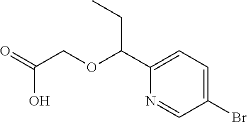

To a solution of 2-(5-bromopyridin-2-yl)propan-2-ol (1.20 g, 4.65 mmol) in dry THE (10 mL) was added NaH (372 mg, 9.30 mmol) slowly at 0° C. and the reaction mixture was stirred at 0° C. for 30 min. Then to the mixture was added ethyl 2-bromoacetate (1.00 g, 6.05 mmol) dropwise, the resulting reaction mixture was warmed to room temperature slowly and stirred at room temperature for 2 h. Then to the reaction mixture was added 1 M NaOH solution (5 mL) and stirred at room temperature for 1 h. The mixture was adjusted with 1 M HCl solution until pH˜4 and extracted with EtOAc (15 mL×3). The combined organic layers were dried over anhydrous Na₂SO₄ and filtered, the filtrate was concentrated under reduced pressure to give the crude product (820 mg) as brown oil. LC-MS (ESI) m z: 274 [M+H]⁺.

### Step 3: 2-(1-(5-bromopyridin-2-yl)propoxy)-1-(pyrrolidin-1-yl)ethenone

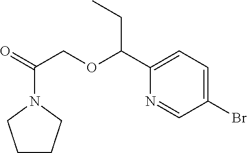 [SMILES: CCC(OCC(=O)N1CCCC1)c1ccc(Br)cn1]

To a mixture of 2-(1-(5-bromopyridin-2-yl)propoxy)acetic acid (820 mg, 3.00 mmol) and pyrrolidine (277 mg, 3.90 mmol) in DCM (10 mL) was added 50% T₃P in EtOAc (3.82 g, 6.00 mmol) followed by DIEA (1.47 mL, 9.00 mmol) at 0° C. And the reaction mixture was stirred at room temperature for 16 h. The mixture was poured into ice sat. NaHCO₃ solution (50 mL) and extracted with DCM (20 mL×2). The organic layer was washed with sat. NH₄Cl solution (50 mL) and brine (50 mL), dried over anhydrous Na₂SO₄ and filtered, the filtrate was concentrated under reduced pressure to give the crude product (452 mg) as brown oil. LC-MS (ESI) m z: 339 [M+H]⁺.

### Step 4: 5-bromo-2-(1-(2-(pyrrolidin-1-yl)ethoxy)propyl)pyridine

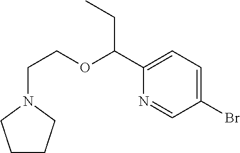 [SMILES: CCC(OCCN1CCCC1)c1ccc(Br)cn1]

To a solution of 2-(1-(5-bromopyridin-2-yl)propoxy)-1-(pyrrolidin-1-yl)ethanone (452 mg, 1.34 mmol) in dry THE (5 mL) was added 1 M BH₃-THF solution (6.70 mL, 6.70 mmol) dropwise at 0° C. and the reaction mixture was stirred at room temperature for 2 h. The mixture was quenched with MeOH (20 mL) and evaporated under reduced pressure. The residue was dissolved in EtOH (30 mL) and stirred at 90° C. for 3 h. The mixture was evaporated under reduced pressure and the residue was purified by chromatography on silica gel (DCM:MeOH=100:1 to 20:1) to give the desired product (111 mg, 6.33% yield for four steps) as light brown oil. LC-MS (ESI) m z: 325 [M+H]⁺.

### Step 5: 1-isopropyl-3-methyl-8-(6-(1-(2-(pyrrolidin-1-yl)ethoxy)propyl)pyridin-3-yl)-1H-imidazo[4,5-c]cinnolin-2(3H)-one

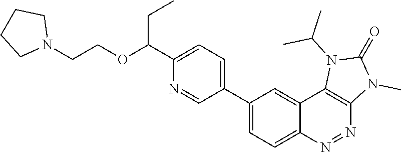 [SMILES: CCC(OCCN1CCCC1)c1ccc(-c2ccc3nnc4c(c3c2)n(C(C)C)c(=O)n4C)cn1]

The crude product was prepared in a similar fashion to Example 1, which was purified by chromatography on silica gel (DCM:MeOH=100:1 to 10:1) to give the desired product (20.0 mg, 12.0% yield) as a light solid. ¹H NMR (400 MHz, CD₃OD) δ 8.98 (d, J=1.9 Hz, 1H), 8.49 (d, J=1.4 Hz, 1H), 8.44 (d, J=9.1 Hz, 1H), 8.33 (dd, J=8.2, 2.4 Hz, 1H), 8.09 (dd, J=9.1, 1.7 Hz, 1H), 7.70 (d, J=8.1 Hz, 1H), 5.41-5.30 (m, 1H), 4.47 (t, J=6.4 Hz, 1H), 3.72-3.54 (m, 5H), 3.04-2.81 (m, 6H), 1.96-1.84 (m, 6H), 1.77 (d, J=6.9 Hz, 6H), 0.98 (t, J=7.4 Hz, 3H). LC-MS (ESI) m/z: 475 [M+H]⁺.

The following compounds were prepared according to the above described methods using different starting materials.

| | | | MS |
|---|---|---|---|
| Ex # | Structure | Name | m/z |
| 220 | 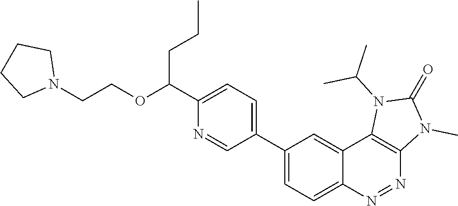 [SMILES: CCCC(OCCN1CCCC1)c1ccc(-c2ccc3nnc4c(c3c2)n(C(C)C)c(=O)n4C)cn1] | 1-isopropyl-3-methyl-8-(6- (1-(2-(pyrrolidin-1- yl)ethoxy)butyl)pyridin-3- yl)-1H-imidazo[4,5- c]cinnolin-2(3H)-one | 489 [M + H]⁺ |
| 221 | 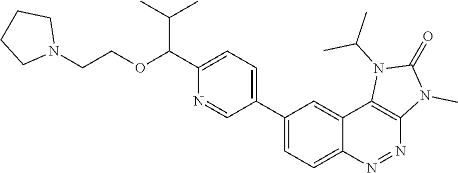 [SMILES: CC(C)C(OCCN1CCCC1)c1ccc(-c2ccc3nnc4c(c3c2)n(C(C)C)c(=O)n4C)cn1] | 1-isopropyl-3-methyl-8-(6- (2-methyl-1-(2-(pyrrolidin- 1-yl)ethoxy)propyl)pyridin- 3-yl)-1H-imidazo[4,5- c]cinnolin-2(3H)-one | 489 [M + H]⁺ |
| 222 | 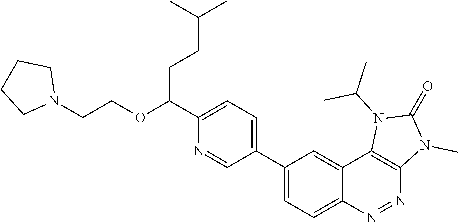 [SMILES: CC(C)CCC(OCCN1CCCC1)c1ccc(-c2ccc3nnc4c(c3c2)n(C(C)C)c(=O)n4C)cn1] | 1-isopropyl-3-methyl-8-(6- (4-methyl-1-(2-(pyrrolidin- 1-yl)ethoxy)pentyl)pyridin- 3-yl)-1H-imidazo[4,5- c]cinnolin-2(3H)-one | 517 [M + H]⁺ |

### Example 220

¹H NMR (400 MHz, CD₃OD) δ 9.09 (d, J=1.9 Hz, 1H), 8.69 (s, 1H), 8.42 (dd, J=8.2, 2.2 Hz, 1H), 8.39 (s, 2H), 7.73 (d, J=8.2 Hz, 1H), 5.53-5.44 (m, 1H), 4.33 (d, J=7.0 Hz, 1H), 3.81-3.72 (m, 2H), 3.70-3.60 (m, 5H), 3.53-3.40 (m, 2H), 3.25-3.09 (m, 2H), 2.25-2.00 (m, 5H), 1.81 (d, J=6.8 Hz, 6H), 1.10 (d, J=6.7 Hz, 3H), 0.88 (d, J=6.8 Hz, 3H).

### Example 221

¹H NMR (400 MHz, CDCl₃) δ 8.92 (d, J=2.0 Hz, 1H), 8.56 (d, J=9.0 Hz, 1H), 8.29 (s, 1H), 8.02 (dd, J=8.1, 2.3 Hz, 1H), 7.90 (dd, J=9.0, 1.7 Hz, 1H), 7.59 (d, J=8.1 Hz, 1H), 5.28-5.18 (m, 1H), 4.19 (d, J=6.5 Hz, 1H), 3.78 (s, 3H), 3.62-3.52 (m, 2H), 2.86-2.73 (m, 2H), 2.63 (s, 4H), 2.21-2.07 (m, 1H), 1.88-1.70 (m, 10H), 1.03 (d, J=6.7 Hz, 3H), 0.91 (d, J=6.8 Hz, 3H).

### Example 222

¹H NMR (400 MHz, CD₃OD) δ 9.00 (d, J=1.9 Hz, 1H), 8.50 (d, J=1.4 Hz, 1H), 8.44 (d, J=9.1 Hz, 1H), 8.34 (dd, J=8.2, 2.3 Hz, 1H), 8.09 (dd, J=9.1, 1.7 Hz, 1H), 7.70 (d, J=8.1 Hz, 1H), 5.40-5.28 (m, 1H), 4.55-4.51 (m, 1H), 3.75-3.66 (m, 4H), 3.64-3.57 (m, 1H), 3.22-2.97 (m, 6H), 2.03-1.82 (m, 6H), 1.77 (d, J=6.9 Hz, 6H), 1.62-1.52 (m, 1H), 1.45-1.36 (m, 1H), 1.26-1.15 (m, 1H), 0.90 (d, J=6.5 Hz, 6H).

### Example 223

### Biological Assays

The efficacy of the compounds of the present disclosure can be determined by a number of pharmacological assays known in the art. The exemplified pharmacological assays, which follow herein, were carried out with the compounds of the present disclosure as well as a control compound 7-fluoro-8-(6-((2-(4-fluoropiperidin-1-yl)ethoxy)methyl)pyridin-3-yl)-1-isopropyl-3-methyl-1H-imidazo[4,5-c]cinnolin-2(3H)-one (Reference compound 1, which was identified in WO2020052688A1 to be a potent inhibitor of ATM kinase): a) ATM biochemical potency assay; b) ATR biochemical potency assay; c) PI3K biochemical potency assay; d) mTOR biochemical potency assay; e) DNA-PK biochemical potency assay; f) phosp-KAP1 MCF-7 cellular potency assay, and g) ATM SN-38 HT-29 cellular potency assay. During the description of the assays, generally:
i. The following abbreviations have been used: 4NQO=4-Nitroquinoline N-oxide; Ab=Antibody; BSA=Bovine Serum Albumin; CO₂=Carbon Dioxide; DMEM=Dulbecco's Modified Eagle Medium; DMSO=Dimethyl Sulphoxide; EDTA=Ethylenediaminetetraacetic Acid; EGTA=Ethylene Glycol Tetraacetic Acid; ELISA=Enzyme-linked ImmunosorbentAssay; EMEM=Eagle's Minimal Essential Medium; FBS=Foetal Bovine Serum; h=Hour(s); HRP=Horseradish Peroxidase; i.p.=intraperitoneal; PBS=Phosphate buffered saline; PBST=Phosphate buffered saline/Tween; TRIS=Tris(Hydroxymethyl)aminomethane; MTS reagent: [3-(4,5-dimethylthiazol-2-yl)-5-(3-carboxymethoxyphenyl)-2-(4-sulfophenyl)-2H-tetrazolium, inner salt, and an electron coupling reagent (phenazine methosulfate) PMS; s.c.=sub-cutaneously.
ii. IC₅₀ values were calculated using a smart fitting model in Genedata. The IC₅₀ value was the concentration of test compound that inhibited 50% of biological activity.

Assay a): ATM Biochemical Potency

ATM (Millipore, Cat. No. 14-933) enzyme solution was prepared in 1× kinase base buffer. 10 μl of 2× enzyme solution was transferred to each well of the 384-well assay plate containing 100 nl compounds added by Echo. The plate was incubated at room temperature for 10 minutes. 2× peptide solution was prepared with FAM-labeled peptide and ATP in the 1× kinase base buffer (final concentration: 1.5 nM). 10 μl of 2× peptide solution was added to each well of the 384-well assay plate which was incubated at 37° C. for 210 min before 40 μl stop buffer was added to stop reaction. Data was collected by Caliper.

Assay b): ATR Biochemical Potency

ATR enzyme (batch: Eurofins Cat. No. 14-953) solutions were prepared in 1× kinase base buffer. 10 μl of 2× enzyme solution (final concentration: 2.5 nM) was added to each well of the 384-well assay plate containing 60 nl compound in each well. The plate was incubated at room temperature for 10 minutes. 2× peptide solutions were prepared with FAM-labeled peptide and ATP in the 1× kinase base buffer. 10 μl of 2× peptide solution was added to each well of the 384-well assay plate, which was incubated at 28° C. for 240 min. 40 μl of stop buffer was added to stop reaction. Data were collected by Caliper.

Assay c): PI3K Biochemical Potency

PI3Kα (p110α/p85a), PIK3C δ, PIK3Cβ (p110β), PIK3Cγ (pp110γ) kinase reaction solutions of PI3Kα (Invitrogen, Cat. No. PV4788), PIK3Cδ (Millipore, Cat. No. 14-604-M), PIK3Cβ (Eurofins, Cat. No. 14-603-K), PIK3Cγ (Invitrogen, Cat. No. PR8641C) enzymes were prepared in 1× kinase buffer at 4-fold of the final concentration (final concentration: PI3Kα 0.7 nM, PIK3C δ 3 nM, PIK3Cβ 4.8 nM, PIK3Cγ 11 nM) of each reagent in the assay. 2.5 μl of kinase solution was added to each well of the 384-well assay plate, which contains 2.5 μl of compounds with serially diluted concentration. 2× substrate solution was prepared with PIP2 substrate and ATP in 1× kinase reaction buffer at 2-fold of the final concentration of each reagent in the assay. 5 μl of substrate solution was added to each well of the assay plate to start reaction. The assay plate was incubated at room temperature for 1 hour. 5 μl reaction mix was transferred to a new 384 well plate. 5 μl of ADP-Glo reagent (Promega, Cat. No. v9102/3) was added to each well of the new assay plate to stop the reaction. The plate was shaken slowly and equilibrated for 40 minutes. 10 μl kinase detection reagents were added to each well, which was equilibrated for 60 minutes before read on a plate reader (Envision) for luminescence.

Assay d): mTOR Biochemical Potency

Solution of mTOR enzymes (Millipore, Cat. No. 14-770) was prepared in 1× kinase buffer at 4-fold of the final concentration (final concentration: 6 nM) in the assay. 2.5 μl of kinase solution was added to each well of the 384-well assay plate, which contains 2.5 μl of compounds with serially diluted concentration. 2× substrate solution was prepared with ULight-4E-BP1 (Thr37/46) Peptide (PE, Cat. No. TRF0128-M) and ATP in 1× kinase reaction buffer at 2-fold of the final concentration of each reagent in the assay. 5 μl of substrate solution was added to each well of the assay plate to start reaction. The assay plate was incubated at room temperature for 30 minutes. Detection solution of kinase quench buffer (EDTA) and Eu-anti-phospho-4E-BP1 antibody (Thr37/46) (PE, Cat. No. TRF0216-M) were prepared at 2-fold the desired final concentrations of each reagent in Lance detection buffer. 10 μl of detection solution buffer was added to each well of the assay plate. The assay plate was equilibrated for 60 minutes at room temperature before read on a plate reader (Lance signal (665 nm) from Envision program).

Assay e): DNA-PK Biochemical Assay

DNA-PK kinase reaction solutions of DNA-PK enzymes (Promega, Cat. No. V4106, Lot. No. 0000224016) were prepared in 1× kinase buffer at 2-fold of the final concentration (final concentration: DNA-PK 1 U/μl, Activator 6 μg/ml) of each reagent in the assay. 2.5 μl of kinase solution was added to each well of the 384-well assay plate, which contains 2.5 μl of compounds with serially diluted concentration. Substrate solution was prepared and ATP in 1× kinase reaction buffer at 2-fold of the final concentration of each reagent in the assay. The final concentration: Substrate 0.2 ug/ml and ATP 20 uM. 2.5 μl of substrate solution was added to each well of the assay plate to start reaction. The assay plate was incubated at room temperature for 1 hour. 5 μl reaction mix was transferred to a new 384 well plate. 5 μl of ADP-Glo MAX reagent 1 (Promega, Cat. No. v9102/3, Lot. No. 0000176563) was added to each well of the assay plate to stop the reaction. The plate was equilibrated for 2 hr at room temperature. 10 μl ADP-Glo MAX reagent 2 was added to each well, which was equilibrated for 30 minutes before read on a plate reader (Envision) for luminescence.

Assay f): phosp-KAP1 MCF-7 Cellular Potency

MCF-7 cells were seeded to a 384-well cell-culture plate, 25 μl/well which were incubated at 37° C. and 5% CO2 for 24 hr. Test compounds were added to the 384-well plates. Etoposide at final concentration of 100 μM was then added to each well except Vehicle control wells, which were incubated at 37° C. and 5% CO2 for 1 hour. After medium removal, cells were fixed by the addition of 25 μl/well of 8% paraformaldehyde in PBSA and incubated for 20 minutes at room temperature. After the plates were rinsed 3 times with PBSA, 50 ul permeabilization buffer (0.1% Triton X-100 in PBS) was added and the plates were incubated for 20 minutes at room temperature. After the plates were rinsed with PBSA, block the cells by adding 50 μl of Odyssey Blocking Buffer in 384-well plate and then incubate for 1.5 hours at room temperature. Remove blocking buffer by Plate washer (BioTek ELx405 select CW). 20 μl/well primary antibody solution (anti-pKAP1 antibody (Bethyl Laboratories, A300-767A)) was added and the plates were incubated in at 4° C. overnight. The plates were rinsed 5 times with PBST (0.1% Tween-20 in PBS). 20 ul/well secondary antibody (IRDye 800CW Goat anti-Rabbit IgG, LI-COR, 926-32211/(IRDye 800CW Goat anti-Mouse IgG, LI-COR, 926-32210) solution containing DNA stain DRAQ5 was added to the plates which were incubated at room temperature for 1 hour away from light. The plates were rinsed 5 times with PBST (0.1% Tween-20 in PBS). After last wash, remove wash solution, turn plate upside down and centrifuge at 1000 rpm for 1 min with paper to absorb all washing buffer. Clean bottom of the plate with moist lint free paper. Scan plate immediately using ODYSSEY CLX (LI-COR) for results.

### Data Analysis:

1) Fluorescence intensity (FI) was detected for each well.
2) % inhibition is calculated as follow:

$$\% inhibition = ⌊ ⌊ \frac{{\overline{FI}}_{Positive} - F {I}_{c m p d}}{{\overline{FI}}_{Positive} - {\overline{FI}}_{Vehicle}} ⌋ ⌋ * 100$$

F̅I̅ₚₒₛᵢₜᵢᵥₑ: The average FI for the positive controls across the plate.
F̅I̅_{vehicle}: The average FI for negative controls across the plate.
3) Calculate IC50 and Plot effect-dose curve of compounds:
   Calculate IC50 by fitting % inhibition values and log of compound concentrations to nonlinear regression (dose response−variable slope) with Graphpad prism 6.0.

Y=Bottom+(Top−Bottom)/(1+10\hat{ }((Log IC50−X)*HillSlope))
X: log of compounds concentration; Y: % inhibition

Assay g): ATM SN-38 HT-29 Cellular Potency

Rationale:

SN38 is an active metabolite of irinotecan, a topoisomerase-I inhibitor. SN38 causes single strand DNA breaks (SSBs) which are converted to double strand breaks (DSBs) during replication. ATM plays role of repairing DSBs. Inhibition of ATM was evaluated in SN38 treated HT-29 cells (ATCC, Cat #HTB-38) by high-Content Imaging System

Experimental Details:

HT-29 cells were trypsinized and approximately 10,000 cells were seeded per well to 96-well microplates which were incubated overnight at 37° C. and 5% CO₂. Test compounds were added to the 96-well plates which were incubated at 37° C. and 5% CO₂ for 1 hour. SN38 (MCE, Cat #HY-13704) at final concentration of 30 nM was then added to the 96-well plates, which were incubated at 37° C. and 5% CO₂ for 1 hour. After medium removal, cells were fixed by the addition of 50 μl 3.7% formaldehyde in PBSA and incubated for 20 minutes at room temperature. After the plates were rinsed 3 times with PBSA, 50 μl permeabilization buffer (0.1% Triton-X 100 in PBSA) was added and the plates were incubated for 20 minutes at room temperature. After the plates were rinsed once with PBSA, 50 μl primary antibody solution was added and the plates were incubated in at 4° C. overnight. The primary antibody solution was prepared by diluting primary antibody (anti-phospho-ATM (Ser1981) antibody, (Merck Millipore, Cat #05-740) at 1/10,000th in antibody buffer (3% BSA, 0.05% Tween in PBSA). The plates were rinsed 3 times with PBST (0.05% Tween in PBSA). 50 μl secondary antibody solution was added to the plates which were incubated at room temperature for 1 hour away from light. The secondary antibody solution was prepared by diluting secondary antibody (Goat anti-Mouse IgG (H+L) Cross-Adsorbed Secondary Antibody, Alexa Fluor 488, Invitrogen, Cat #A11001) at 1/500th and Hoechst at 1/10,000th in antibody buffer. The plates were rinsed with PBST 3 times and then 100 μl PBSA were added per well. The plates were sealed with black plate seals.

Data Capture

| Instrument | High-Content Imaging System, ImageXpress (Molecular | | |
|---|---|---|---|
| | Devices) | | |
| Software | ImageXpress (5.1.0.3) | | |
| Plate | 96-well cell culture plate, μ clear - Grenier - 655090 | | |
| Objective | 10X | | |
| Well | 9 sites/well | | |
| | 300 μM between images in X direction and 300 μM in Y | | |
| | direction | | |
| Wavelength | 2 Wavelengths | W1 DAPI (Nuclei) | Auto Exposure |
| | | W2 FITC (pATM) | Auto Exposure |

| | | |
|---|---|---|
| Focus | Focusing each | Autofocus at each site in well |
| | well | |

Data Analysis

| | |
|---|---|
| Software | MetaXpress (5.1.0.41) |
| Module | Cell Scoring |
| | Measures positive cells in assay with a nuclear stain |
| | and a probe of interest |
| Parameters | “Cell Number” & “Positive Cells Area” |

After image analysis, further statistics were done by Excel 2013 (Microsoft). The graphical views were then generated using Prism 7.0 (Graphpad).

The compounds synthesized in Examples 1-222 and Reference compound 1 were tested in Assays a)-g) as described above. The IC₅₀ results are provided in Table 2-3 for some representative compounds.

TABLE 2
Potency Data for Representative
Compounds in Assays a)

| | ATM IC₅₀ |
|---|---|
| Example | (nM) |
| 1 | 16 |
| 2 | 53 |
| 3 | 36 |
| 4 | 63 |
| 5 | 14 |
| 6 | 469 |
| 7 | 227 |
| 8 | 98 |
| 9 | 180 |
| 10 | 105 |
| 11 | 123 |
| 12 | 47 |
| 13 | 144 |
| 14 | 68 |
| 15 | 134 |
| 16 | 201 |
| 17 | 464 |
| 18 | 377 |
| 19 | 99 |
| 20 | 51 |
| 21 | 139 |
| 23 | 420 |
| 24 | 26 |
| 25 | 71 |
| 26 | 14 |
| 27 | 92 |
| 28 | 81 |
| 29 | 68 |
| 30 | 62 |
| 31 | 11 |
| 32 | 304 |
| 33 | 58 |
| 34 | 47 |
| 35 | 43 |
| 36 | 4.7 |
| 37 | 78 |
| 38 | 57 |
| 39 | 73 |
| 40 | 24 |
| 41 | 50 |
| 42 | 44 |
| 43 | 12 |
| 44 | 12 |
| 45 | 84 |
| 46 | 76 |
| 47 | 2.2 |
| 48 | 17 |
| 49 | 2.8 |
| 50 | 11 |
| 51 | 7.6 |
| 53 | 4.2 |
| 54 | 3.2 |
| 55 | 3.2 |
| 56 | 3.2 |
| 57 | 4.2 |
| 58 | 1.4 |
| 59 | 8.3 |
| 60 | 2.8 |
| 61 | 3.4 |
| 62 | 7.8 |
| 64 | 19 |
| 65 | 167 |
| 66 | 10 |
| 67 | 158 |
| 68 | 160 |
| 69 | 5.2 |
| 70 | 295 |
| 71 | 9.3 |
| 72 | 50 |
| 73 | 3.5 |
| 74 | 179 |
| 75 | 19 |
| 76 | 2.1 |
| 77 | 4.8 |
| 78 | 6.4 |
| 79 | 37 |
| 81 | 3.0 |
| 82 | 3.4 |
| 83 | 2.4 |
| 84 | 1.9 |
| 85 | 3.4 |
| 86 | 3.6 |
| 87 | 8.2 |
| 89 | 1.6 |
| 90 | 11 |
| 91 | 8.5 |
| 92 | 1.5 |
| 93 | 4.9 |
| 94 | 24 |
| 95 | 47 |
| 96 | 17 |
| 97 | 14 |
| 98 | 62 |
| 99 | 40 |
| 100 | 16 |
| 101 | 3.7 |
| 102 | 5.5 |
| 103 | 3.9 |
| 104 | 19 |
| 105 | 13 |
| 106 | 4.2 |
| 107 | 2.8 |
| 108 | 3.7 |
| 109 | 2.1 |
| 110 | 6.9 |
| 111 | 12 |
| 112 | 7.8 |
| 113 | 7.5 |
| 116 | 2.9 |
| 117 | 3.7 |
| 118 | 4.1 |
| 119 | 2.4 |
| 120 | 11 |
| 121 | 13 |
| 122 | 4.3 |
| 123 | 16 |
| 124 | 1.1 |
| 125 | 6.3 |
| 126 | 20 |
| 127 | 13 |
| 128 | 4.6 |
| 129 | 75 |
| 130 | 26 |
| 132 | 22 |
| 133 | 13 |
| 135 | 4.4 |
| 136 | 10 |
| 137 | 6.3 |
| 138 | 14 |
| 139 | 2.8 |
| 140 | 2.8 |
| 141 | 6.3 |
| 142 | 4.7 |
| 143 | 11 |
| 144 | 2.9 |
| 147 | 23 |
| 148 | 13 |
| 149 | 15 |
| 153 | 8.5 |
| 154 | 3.6 |
| 155 | 11 |
| 156 | 6 |
| 159 | 20.6 |
| 160 | 13.3 |
| 161 | 3.4 |
| 162 | 7 |
| 163 | 7.9 |
| 164 | 2.2 |
| 165 | 101 |
| 166 | 58 |
| 167 | 56 |
| 168 | 48 |
| 169 | 42 |
| 170 | 1.6 |
| 171 | 2.5 |
| 172 | 21 |
| 173 | 32 |
| 174 | 28 |
| 175 | 25 |
| 176 | 65 |
| 179 | 729 |
| 182 | 21 |
| 183 | 20 |
| 184 | 2.8 |
| 185 | 292 |
| 216 | 339 |
| 217 | 72 |
| 219 | 57 |
| 220 | 147 |
| 221 | 156 |
| Ref | 25 |
| compd 1 | |

TABLE 3
Potency Data for representative compounds in Assays b)-g)

| | | | | | | | | Etoposide | SN-38 |
|---|---|---|---|---|---|---|---|---|---|
| | ATR | PI3Kα | PI3Kβ | PI3Kγ | PI3Kδ | mTor | DNA-PK | MCF-7 cellular | HT-29 cellular |
| | IC₅₀ | IC₅₀ | IC₅₀ | IC₅₀ | IC₅₀ | IC₅₀ | IC₅₀ | IC₅₀ | IC₅₀ |
| Example | (μM) | (μM) | (μM) | (μM) | (μM) | (μM) | (μM) | (nM) | (nM) |
| 1 | | | | | | | | 40 | |
| 5 | | | | | | | | 27 | 130 |
| 31 | >10 | >10 | >10 | >10 | >10 | >10 | >10 | 16 | 29 |
| 36 | | | | | | | | 20 | 20 |
| 43 | | | | | | | | | 92 |
| 53 | >10 | >10 | >10 | >10 | >10 | >10 | >10 | 9 | 11 |
| 54 | | | | | | | | 17 | |
| 55 | | | | | | | | 11 | |
| 56 | >10 | >10 | >10 | >10 | >10 | >10 | >10 | | |
| 57 | | | | | | | | 20 | 19 |
| 59 | | | | | | | | 65 | |
| 62 | >10 | >10 | >10 | >10 | >10 | >10 | >10 | | 21 |
| 69 | | | | | | | | 34 | 47 |
| 73 | | | | | | | | 9.1 | 9.1 |
| 76 | >10 | >10 | >10 | >10 | >10 | >10 | >10 | | |
| 77 | >10 | >10 | >10 | >10 | >10 | >10 | >10 | | |
| 86 | | | | | | | | 12 | |
| 97 | | | | | | | | 43 | |
| 102 | >10 | >10 | >10 | >10 | >10 | >10 | >10 | 12 | 17 |
| 103 | | | | | | | | 20 | |
| 105 | | | | | | | | 36 | |
| 107 | >10 | >10 | >10 | >10 | >10 | >10 | >10 | 12 | 15 |
| 110 | | | | | | | | 13 | |
| 112 | >10 | >10 | >10 | >10 | >10 | >10 | >10 | 20 | 18 |
| 117 | | | | | | | | 65 | |
| 118 | | | | | | | | 71 | |
| 133 | | | | | | | | 44 | |
| 137 | | | | | | | | 22 | |
| 140 | | | | | | | | 14 | |
| 142 | | | | | | | | 24 | |
| 144 | | | | | | | | 47 | |
| 153 | | | | | | | | 43 | |
| 162 | >10 | >10 | >10 | >10 | >10 | >10 | >10 | | 5.5 |
| 174 | | | | | | | | 26 | |
| 175 | | | | | | | | 33 | 170 |
| 184 | >10 | >10 | >10 | >10 | >10 | >10 | >10 | | |
| 185 | >10 | >10 | >10 | >10 | >10 | >10 | >10 | | |
| Ref | | | | | | | | | 273 |
| compd 1 | | | | | | | | | |

From Table 2-3, it can be found that the compounds of the present disclosure not only have good inhibition of ATM kinase, but also selective for ATM kinase over other kinases (PI3Kα, PI3Kβ, PI3Kγ, PI3Kδ, mTor, DNA-PK and ATR) in PIKK family.

For the other Example compounds for which the results are not shown, all have an IC₅₀ against ATM kinase of no more than 1000 nM. Some of these compounds have an IC₅₀ against ATM kinase of no more than 500 nM, some no more than 400 nM, some no more than 300 nM, some no more than 200 nM, or no more than 100 nM, or even no more than 50 nM. In addition, some of the Example compounds for which the results are not shown show IC₅₀ against other kinases (PI3Kα, PI3Kβ, PI3Kγ, PI3Kδ, mTor and ATR) in PIKK family of more than 1 μM, some more than 3 μM, more than 5 μM, more than 7 μM, or even more than 10 μM.

### Example 224

### AO Activity Study

AO activity study was carried out with the compounds of the present disclosure and Reference compounds 2, 3, 4 (Zaleplon, an AO assay positive control) and 5 (PF-04217903, a weak AO substrate) using the following assay h) aldehyde oxidase assay.

Assay h): Aldehyde Oxidase Assay

AO activity was assessed in human liver cytosol. The incubation system was composed of phosphate buffer, human liver cytosol and test compounds or positive controls. The reaction was stopped at 0.5, 15, 30, 60, 90 and 120 minutes by the addition of 5 volumes of cold acetonitrile with internal standard. Samples were centrifuged at 3, 220 g for 30 minutes. Aliquot of 100 μL of the supernatant was mixed with 100 μL of ultra-pure H₂O and then used for LC/MS/MS analysis.

All calculations were carried out using Microsoft Excel. Peak areas were determined from extracted ion chromatograms. The slope value, k, was determined by linear regression of the natural logarithm of the remaining percentage of the parent drug vs. incubation time curve. The in vitro half-life (in vitro tv₂) was determined from the slope value using the following equation:

in vitro t_{1/2}: =−(0.693/k)

Conversion of the in vitro t_{1/2} (min) into the in vitro intrinsic clearance (in vitro CLᵢₙₜ, in μL/min/mg protein) was done using the following equation (mean of duplicate):

The compounds synthesized in Examples 1-222 and Reference compounds 2-4 were tested in Assay h) as described above. Results of representative compounds of the present disclosure and Reference compounds 2-5 are shown in Table 4.

TABLE 4
Results of representative compounds and
Reference compounds 2-5 in assay h)

| | AO intrinsic clearance |
|---|---|
| Example | (μL/min/mg protein) |
| 53 | <0.1 |
| 62 | 0.19 |
| 102 | 0.17 |
| 107 | <0.1 |
| 112 | <0.1 |
| Ref. Comp. 2 | 1.44 |
| Ref. Comp. 3 | 2.20*(1.90~2.49) |
| Ref. Comp. 4 | 0.893 ± 0.179** |
| Ref. Comp. 5 | 0.413 ± 0.169** |

*Average results of duplicate test;
**Mean ± SD

It is demonstrated Reference compounds 2 and 3 are shown to be strong AO substrates with intrinsic clearance of 1.44 and 2.20 μL/min/mg protein, higher than that of Reference compound 4 (0.893 μL/min/mg protein). Reference compound 4 showed high clearance in humans at 16 mL/min/kg corresponding to ˜80% liver blood flow (Zientek, M. et al, Drug MetabDispos 2010, 1322-7). In contrast, the compounds of the present disclosure show an intrinsic clearance lower than that of Reference compound 5 (0.413 μL/min/mg protein). Reference compound 5 showed low to moderate clearance in humans at 6 mL/min/kg corresponding to ˜₃₀% liver blood flow. This indicates that the compounds of the present disclosure are not AO substrates. For the other Example compounds for which the results are not shown, all show intrinsic clearance lower than that of Reference compound 5.

### Example 225

### Efficacy Study

The therapeutic relevance of ATM inhibition by representative compounds were investigated in vivo in combination with ionizing radiation (IR), a clinically established DSB-inducing treatment. Representative compounds were tested for activity in xenograft mouse model of human cancer using the following assay i).

Assay i): FaDu Xenograft Model

Radiation Combination

Cell culture: FaDu tumor cells were cultured in an EMEM medium containing inactivated 10% fetal bovine serum, 100 U/ml penicillin and 100 μg/ml streptomycin in a 37° C., 5% CO₂ incubator.

Inoculation and grouping: Xenografts were established by implantation of 100 μl of Fadu tumor cell suspension (5×10⁶ cells/mouse) subcutaneously into the right flank of female immune-compromised BALB/c nude mice at least 6 weeks of age. Tumours were measured in two dimensions with calipers and the tumour volume was calculated using the following formula: tumor volume=(length×width²)×0.5. When the average tumor volume reached 100 to 150 mm³, tumour-bearing mice were randomized into treatment groups.

Treatment and tumor assessment: Animals received 3 to 6 cycles of treatment with compounds combined with radiation therapy. Each cycle included 5 consecutive treatment day plus 2 days' rest. Ionizing radiation was administered using a fractionated schedule of 2 Gy per day administered over 5 consecutive days (total radiation dose=10 Gy/cycle). Compound was given orally 10 minutes before irradiation. The tumour volume and body weight of the mice were measured twice weekly. Tumor growth inhibition (0% TGI) from start of treatment was assessed by comparison of the mean change in tumor volume for the control and treated groups. When the tumor growth inhibition was more than 10000, it meant the treatment induced tumor regression.

Statistical Analysis: The One-Way ANOVA test was used for statistical analysis of tumor volume between groups, and p<0.05 was considered as a significant difference.

TABLE 5
Summary of Results in Assay i) after Exposure to Different Doses of Radiation and Representative Compounds

| Total IR | | Dose | Route & | Inhibition (% TGI) | | P value |
|---|---|---|---|---|---|---|
| dose (Gy) | Example | (mg/kg) | Freq | IR only | Combo | (vs. IR only) |
| 30 | 5 | 50 | PO. TID | 66.6 ± 3.32 | 79.6 ± 2.78 | 0.0057 |
| (3 cycles) | | | | | | |
| 30 | 5 | 150 | PO. TID | 66.6 ± 3.32 | 91.9 ± 1.16 | <0.0001 |
| (3 cycles) | | | | | | |
| 30 | 1 | 50 | PO. TID | 66.6 ± 3.32 | 76.8 ± 2.72 | 0.0205 |
| (3 cycles) | | | | | | |
| 30 | 1 | 150 | PO. TID | 66.6 ± 3.32 | 92.0 ± 1.31 | <0.0001 |
| (3 cycles) | | | | | | |
| 30 | 102 | 25 | PO. BID | 75.5 ± 1.85 | 83.0 ± 3.03 | 0.19 |
| (3 cycles) | | | | | | |
| 40 | 102 | 50 | PO. BID | 75.5 ± 1.85 | 93.4 ± 1.75 | <0.0001 |
| (4 cycles) | | | | | | |
| 40 | 102 | 75 | PO. BID | 75.5 ± 1.85 | 96.4 ± 0.91 | <0.0001 |
| (4 cycles) | | | | | | |
| 40 | 112 | 30 | PO. BID | 75.5 ± 1.85 | 77.3 ± 1.67 | 0.78 |
| (4 cycles) | | | | | | |
| 40 | 112 | 60 | PO. BID | 75.5 ± 1.85 | 79.6 ± 4.20 | 0.44 |
| (4 cycles) | | | | | | |
| 40 | 112 | 100 | PO. BID | 75.5 ± 1.85 | 82.9 ± 1.77 | 0.21 |
| (4 cycles) | | | | | | |
| 60 | 62 | 10 | PO. BID | 64.4 ± 3.91 | 80.4 ± 2.20 | <0.0001 |
| (6 cycles) | | | | | | |
| 60 | 62 | 30 | PO. BID | 64.4 ± 3.91 | 93.6 ± 1.33 | <0.0001 |
| (6 cycles) | | | | | | |
| 30 | 62 | 45 | PO. BID | 64.4 ± 3.91 | 98.7 ± 0.51 | <0.0001 |
| (3 cycles) | | | | | | |
| 30 | 102 | 20 | PO. BID | 64.4 ± 3.91 | 83.5 ± 2.21 | <0.0001 |
| (3 cycles) | | | | | | |
| 30 | 102 | 40 | PO. BID | 64.4 ± 3.91 | 93.7 ± 1.04 | <0.0001 |
| (3 cycles) | | | | | | |
| 30 | 102 | 60 | PO. BID | 64.4 ± 3.91 | 95.5 ± 1.63 | <0.0001 |
| (3 cycles) | | | | | | |

The Palliative radiotherapy regiment used in the clinic is a 3-4 week fractionated treatment schedule (5 fractions of 2 Gy IR per week-30-40 Gy total). The standard radiotherapy regiment used in the clinic is a 6 week fractionated treatment schedule (5 fractions of 2 Gy IR per week-60 Gy total). To examine the therapeutic potential of compounds, these two regiments were applied in Fadu xenograft models of human head and neck cancer.

In the FaDu xenograft efficacy study, the combination treatment with both IR and the compound of the present disclosure resulted in a strong inhibition of tumor growth in comparison to the treatment with IR alone. For representative compounds (Example 62 and 102) in combination with IR, tumor xenograft regression was observed and tumors did not relapse until the end of the study.

### Example 226

### Dose Range Finding Study

The oral toxicities of representative compounds are assessed in repeat-dose toxicity studies in rats, dogs and/or monkeys using the following assays:

Assay j): dose Range Finding Studies

In rat dose range finding (DRF) study, rats are randomly assigned to 4 groups, including control group, test compound low dose group, test compound middle dose group and test compound high dose group. Control group is composed of 10 rats (5/sex), and each test compound group has 16 rats (8/sex, in which 3/sex of them were used for toxicokinetics). Animals are dose consecutively for 28 days. Clinical signs, body weight and food consumption are measured daily or twice weekly. At the end of the study, rats are anesthetized, sampled for hematology and clinical chemistry, followed by gross necropsy observation and organ weight measurement. On the first day and last day of dosing, blood samples are collected from toxicokinetic (TK) animals of test compound groups to verify the TK profiles and obtain TK parameters of test compound.

In dog or monkey DRF study, animals are randomly assigned to 4 groups, including control group, test compound low dose group, test compound middle dose group and test compound high dose group, and each group is composed of 4 animals (2/sex). Animals are dose consecutively for 28 days. Clinical signs, body weight and food consumption are measured daily or twice weekly. At the end of the study, animals are anesthetized, sampled for hematology and clinical chemistry, followed by gross necropsy observation and organ weight measurement. On the first day and last day of dosing, blood samples are collected from each animal of test compound groups to verify the TK profiles and obtain TK parameters of test compound.

The foregoing description is considered as illustrative only of the principles of the present disclosure. Further, since numerous modifications and changes will be readily apparent to those skilled in the art, it is not desired to limit the invention to the exact construction and process shown as described above. Accordingly, all suitable modifications and equivalents maybe considered to fall within the scope of the invention as defined by the claims that follow.

The words “comprise,” “comprising,” “include,” “including,” and “includes” when used in this specification and in the following claims are intended to specify the presence of stated features, integers, components, or steps, but they do not preclude the presence or addition of one or more other features, integers, components, steps, or groups thereof.

1-64. (canceled)

65. A compound of formula (I): 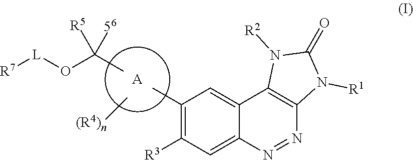
or a pharmaceutically acceptable salt thereof,
wherein:
Ring A is aryl or heteroaryl;
R¹ is hydrogen or methyl optionally substituted with 1, 2, or 3 halogens;
R² is selected from the group consisting of alkyl, alkenyl, alkynyl, cycloalkyl, heterocyclyl, aryl, and heteroaryl, wherein said alkyl, alkenyl, alkynyl, cycloalkyl, heterocyclyl, aryl, and heteroaryl are optionally substituted with one or more R⁸;
R³ is selected from the group consisting of hydrogen, halogen, hydroxyl, cyano, alkyl, alkenyl, alkynyl, haloalkyl and cycloalkyl;
each R⁴ is independently selected from hydrogen, halogen, alkyl, alkenyl, alkynyl, and cycloalkyl;
R⁵ and R⁶ are each independently selected from the group consisting of hydrogen, halogen, cyano, alkyl, alkenyl, alkynyl, haloalkyl and cycloalkyl; or
R⁵ and R⁶ together with the carbon atom to which they are attached form cycloalkyl or heterocyclyl, said cycloalkyl or heterocyclyl are optionally substituted with one or more R⁹;
L is selected from a direct bond, alkyl, cycloalkyl or cycloalkylalkyl, wherein said alkyl, cycloalkyl, and cycloalkylalkyl are optionally substituted with one or more R⁹;
R⁷ is selected from the group consisting of —NR¹⁰R¹¹, —OR¹², —COOH, a saturated or unsaturated heterocyclyl optionally substituted with one or more R¹³;
R⁸ is selected from the group consisting of hydrogen, hydroxyl, halogen, cyano, alkyl, alkenyl, alkynyl, haloalkyl, and alkoxyl;
R⁹ is selected from the group consisting of hydrogen, halogen, alkyl, haloalkyl, alkenyl, alkynyl, cycloalkyl, and heterocyclyl;
R¹⁰ and R¹¹ are each independently selected from the group consisting of hydrogen, alkyl, alkenyl, alkynyl, haloalkyl and cycloalkyl; or
R¹⁰ and R¹¹ together with the nitrogen atom to which they are attached form heterocyclyl optionally containing one or more additional heteroatoms selected from N, O and S and optionally substituted with one or more R¹⁴;
R¹² and R¹³ are each independently selected from the group consisting of alkyl, alkenyl, alkynyl, haloalkyl, cycloalkyl, and heterocyclyl;
R¹⁴ is selected from the group consisting of halogen, cyano, sulfonyl, alkyl, alkenyl, alkynyl, haloalkyl, alkylalkoxyl, heterocyclyl, —NR¹⁵R¹⁶, and —OR¹⁷, R¹⁵ and R¹⁶ are each independently hydrogen, alkyl, alkenyl, or alkynyl; and
R¹⁷ is selected from the group consisting of alkyl, alkenyl, alkynyl, haloalkyl, and cycloalkyl, and
n is 0, 1, 2 or 3.

66. The compound of Formula (I) or a pharmaceutically acceptable salt thereof, as claimed in claim 65, wherein Ring A is selected from the group consisting of thienyl, furanyl, pyrrolyl, imidazolyl, pyrazolyl, oxazolyl, isoxazolyl, thiazolyl, isothiazolyl, pyridyl, pyridazinyl, pyrimidinyl and pyrazinyl.

67. The compound of Formula (I) or a pharmaceutically acceptable salt thereof, as claimed in claim 65, wherein R¹ is methyl optionally substituted with 1, 2, or 3 halogens.

68. The compound of Formula (I) or a pharmaceutically acceptable salt thereof, as claimed in claim 65, wherein R² is selected from any one of (a) to (d):
(a) R² is alkyl optionally substituted with one or more R⁸, and R⁸ is selected from the group consisting of hydrogen, hydroxyl, halogen, cyano, and alkoxyl, preferably R² is n-propyl or iso-propyl;
(b) R² is cycloalkyl optionally substituted with one or more R⁸, and R⁸ is selected from the group consisting of hydrogen, hydroxyl, halogen, cyano, alkyl, haloalkyl, and alkoxyl, preferably R² is selected from the group consisting of cyclopropyl, cyclobutyl, cyclopentyl and cyclohexyl, each of which is optionally substituted with one or more R⁸, and R⁸ is selected from hydroxyl, methyl, trifluoromethyl or methoxy, preferably R² is 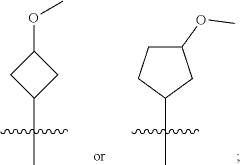
(c) R² is heterocyclyl optionally substituted with one or more R⁸, and R⁸ is selected from the group consisting of hydrogen, hydroxyl, halogen, cyano, alkyl, haloalkyl and alkoxyl, preferably R² is selected from the group consisting of oxetanyl, tetrahydrofuranyl and tertrahydropyranyl, each of which is optionally substituted with one or more R⁸, and R⁸ is selected from the group consisting of hydroxyl, methyl, trifluoromethyl and methoxy, preferably R² is 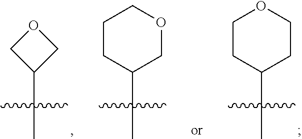
(d) R² is heteroaryl optionally substituted with one or more R⁸, and R⁸ is selected from the group consisting of hydrogen, hydroxyl, halogen, cyano, alkyl, haloalkyl and alkoxyl, preferably R² is pyridine or pyrazole, each of which is optionally substituted with one or more R⁸, and R⁸ is selected from the group consisting of hydroxyl, methyl, trifluoromethyl and methoxy.

69. The compound of Formula (I) or a pharmaceutically acceptable salt thereof, as claimed in claim 65, wherein R³ is hydrogen or fluoro.

70. The compound of Formula (I) or a pharmaceutically acceptable salt thereof, as claimed in claim 65, wherein R⁴ is hydrogen.

71. The compound of Formula (I) or a pharmaceutically acceptable salt thereof, as claimed in claim 65, wherein R⁵ and R⁶ are selected from any one of (a) to (c):
(a) one of R⁵ and R⁶ is hydrogen, and the other is alkyl or haloalkyl, preferably one of R⁵ and R⁶ is hydrogen, and the other is C₁₋₆ alkyl or C₁₋₆ haloalkyl, preferably one of R⁵ and R⁶ is hydrogen, and the other is methyl or trifluoromethyl;
(b) both of R⁵ and R⁶ are hydrogen or alkyl;
(c) R⁵ and R⁶ together with the carbon atom to which they are attached form cycloalkyl or heterocyclyl.

72. The compound of Formula (I) or a pharmaceutically acceptable salt thereof, as claimed in claim 65, wherein L is selected from any one of (a) to (c):
(a) L is a direct bond or cycloalkylalkyl;
(b) L is alkyl optionally substituted with one or more R⁹, and each R⁹ is independently selected from the group consisting of hydrogen, halogen, and cycloalkyl; or
(c) L is cycloalkyl optionally substituted with one or more R⁹, and each R⁹ is independently selected from the group consisting of hydrogen, halogen, alkyl, haloalkyl and cycloalkyl.

73. The compound of Formula (I) or a pharmaceutically acceptable salt thereof, as claimed in claim 65, wherein R⁷ is —NR¹⁰R¹¹.

74. The compound of Formula (I) or a pharmaceutically acceptable salt thereof, as claimed in claim 73, wherein R¹⁰ and R¹¹ are selected from any one of (a) to (c):
(a) R¹⁰ and R¹¹ are each independently alkyl, or one of R¹⁰ and R¹¹ is hydrogen, and the other is alkyl; or
(b) R¹⁰ and R¹¹ together with the nitrogen atom to which they are attached form a saturated heterocyclyl optionally containing one or more additional heteroatoms selected from N, O and S and optionally substituted with one or more R¹⁴; or
(c) R¹⁰ and R¹¹ together with the nitrogen atom to which they are attached form a saturated heterocyclyl selected from the group consisting of: 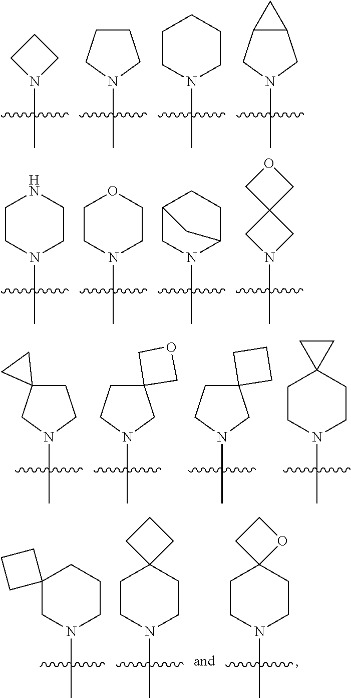
each of which is optionally substituted with one or more R¹⁴, preferably R¹⁴ is independently selected from the group consisting of halogen, cyano, sulfonyl, alkyl, haloalkyl, alkylalkoxyl, —NR¹⁵R¹⁶, and OR¹⁷, wherein R¹⁵ and R¹⁶ are each independently alkyl, or one of R¹⁵ and R¹⁶ is hydrogen, and the other is alkyl, and R¹⁷ is selected from alkyl or haloalkyl.

75. The compound of Formula (I) or a pharmaceutically acceptable salt thereof, as claimed in claim 64, wherein R⁷ is any one of (a) to (b):
(a) R⁷ is —COOH; or
(b) R⁷ is heterocyclyl optionally substituted with R¹³, and R¹³ is alkyl, preferably R⁷ is selected from the group consisting of: 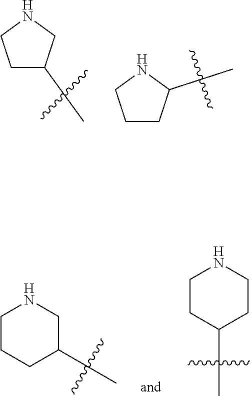 [SMILES: *C1CCNC1 ; *C1CCCN1 ; *C1CCCNC1 ; *C1CCNCC1]
each of which is optionally substituted with one or more R¹³.

76. The compound of Formula (I) or a pharmaceutically acceptable salt thereof, as claimed in claim 65, wherein the compound has a formula selected from the group consisting of: 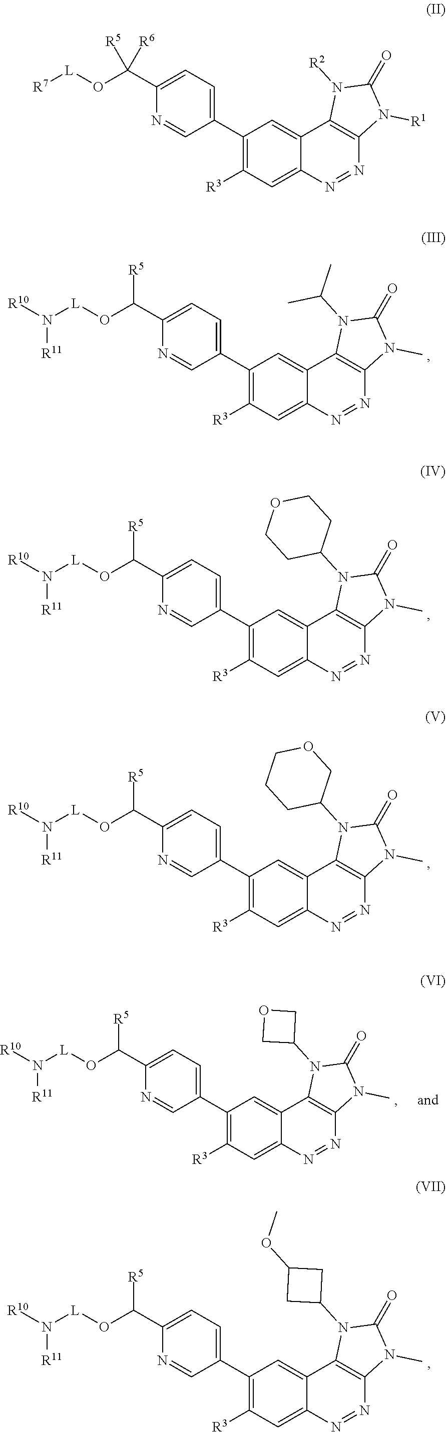
wherein R¹, R², R³, R⁵, R⁶, R⁷, L, R¹¹ and R¹² are as defined in claim 1.

77. The compound of Formula (I) or a pharmaceutically acceptable salt thereof, as claimed in claim 76, wherein:
R³ is hydrogen or fluoro;
R⁵ is hydrogen, alkyl or haloalkyl, preferably R⁵ is hydrogen, methyl or trifluoromethyl;
L is alkyl optionally substituted with one or more R⁹, preferably L is ethyl or propyl;
R⁹ is selected from the group consisting of hydrogen, halogen, and cycloalkyl;
R¹⁰ and R¹¹ are each independently alkyl, or one of R¹⁰ and R¹¹ is hydrogen, and the other is alkyl;
R¹⁰ and R¹¹ together with the nitrogen atom to which they are attached form a saturated heterocyclyl optionally containing one or more additional heteroatoms selected from N, O and S and optionally substituted with one or more R¹⁴;
R¹⁴ is selected from the group consisting of halogen, cyano, sulfonyl, alkyl, haloalkyl, alkylalkoxyl, a saturated or unsaturated heterocyclyl, —NR¹⁵R¹⁶, and —OR¹⁷;
R¹⁵ and R¹⁶ are each independently alkyl, or one of R¹⁵ and R¹⁶ is hydrogen, and the other is alkyl, and
R¹⁷ is selected from alkyl or haloalkyl.

78. The compound of Formula (I) or a pharmaceutically acceptable salt thereof, as claimed in claim 65, wherein the compound has a formula selected from the group consisting of: 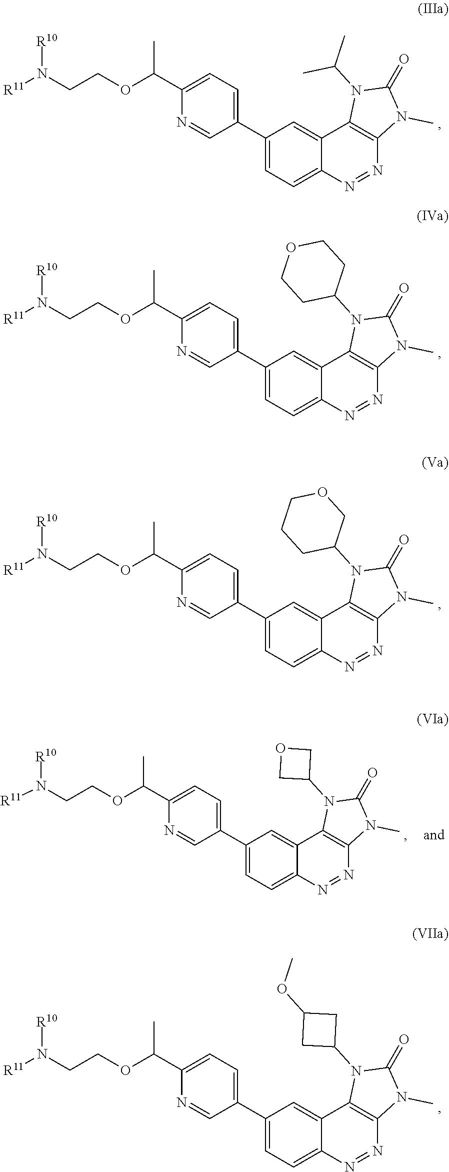 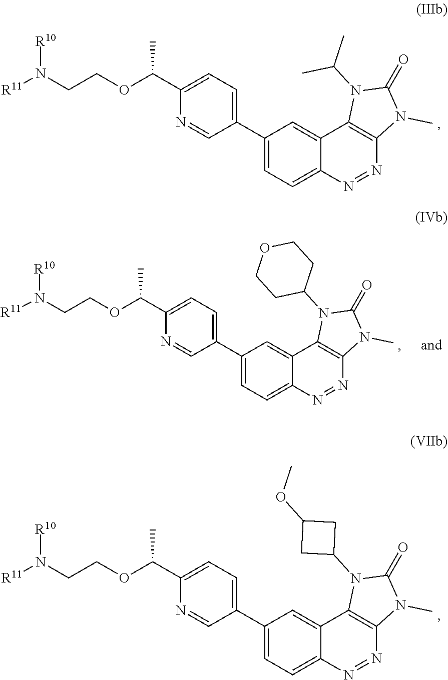
wherein R¹⁰ and R¹¹ are as defined in claim 1.

79. The compound of Formula (I) or a pharmaceutically acceptable salt thereof, as claimed in claim 78, wherein R¹⁰ and R¹¹ together with the nitrogen atom to which they are attached form a saturated heterocyclyl selected from the group consisting of:

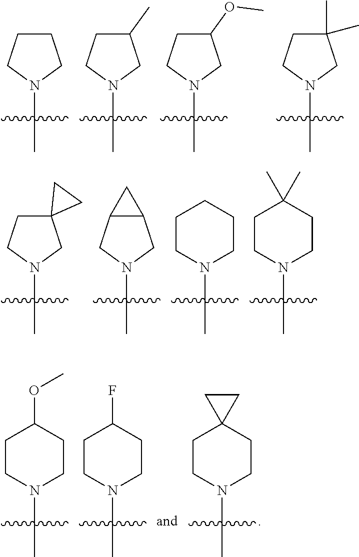

80. The compound of Formula (I) or a pharmaceutically acceptable salt thereof, as claimed in claim 65, wherein the compound is selected from the group consisting of:
8-(6-((2-(3-azabicyclo[3.1.0]hexan-3-yl)ethoxy)methyl)pyridin-3-yl)-1-isopropyl-3-methyl-1H-imidazo[4,5-c]cinnolin-2(3H)-one
8-(6-((2-(dimethylamino)ethoxy)methyl)pyridin-3-yl)-1-isopropyl-3-methyl-1H-imidazo[4,5-c]cinnolin-2(3H)-one
1-isopropyl-3-methyl-8-(6-((2-(piperidin-1-yl)ethoxy)methyl) pyridin-3-yl)-1H-imidazo[4,5-c]cinnolin-2(3H)-one
1-isopropyl-3-methyl-8-(6-((2-(pyrrolidin-1-yl)ethoxy) methyl)pyridin-3-yl)-1H-imidazo[4,5-c]cinnolin-2(3H)-one
8-(6-((2-(4-fluoropiperidin-1-yl)ethoxy)methyl)pyridin-3-yl)-1-isopropyl-3-methyl-1H-imidazo[4,5-c]cinnolin-2(3H)-one
1-isopropyl-8-(6-((2-methoxyethoxy)methyl)pyridin-3-yl)-3-methyl-1H-imidazo[4,5-c]cinnolin-2(3H)-one
1-isopropyl-3-methyl-8-(6-(((1-methylpiperidin-4-yl)oxy)methyl) pyridin-3-yl)-1H-imidazo[4,5-c]cinnolin-2(3H)-one
8-(6-((3-(dimethylamino)propoxy)methyl)pyridin-3-yl)-1-isopropyl-3-methyl-1H-imidazo[4,5-c]cinnolin-2(3H)-one
1-isopropyl-3-methyl-8-(6-(((1-methylpiperidin-4-yl)methoxy) methyl)pyridin-3-yl)-1H-imidazo[4,5-c]cinnolin-2(3H)-one
1-isopropyl-3-methyl-8-(6-(((1-methylpyrrolidin-2-yl)methoxy) methyl)pyridin-3-yl)-1H-imidazo[4,5-c]cinnolin-2(3H)-one
1-isopropyl-3-methyl-8-(6-(((1-methylpyrrolidin-3-yl)oxy) methyl)pyridin-3-yl)-1H-imidazo[4,5-c]cinnolin-2(3H)-one
8-(6-((3-(3-azabicyclo[3.1.0]hexan-3-yl)propoxy)methyl)pyridin-3-yl)-1-isopropyl-3-methyl-1H-imidazo[4,5-c]cinnolin-2(3H)-one
1-isopropyl-3-methyl-8-(6-(((1-methylpiperidin-3-yl)oxy)methyl)pyridin-3-yl)-1H-imidazo[4,5-c]cinnolin-2(3H)-one
8-(6-((3-(dimethylamino)cyclobutoxy)methyl)pyridin-3-yl)-1-isopropyl-3-methyl-1H-imidazo[4,5-c]cinnolin-2(3H)-one
1-isopropyl-3-methyl-8-(6-(((1-methylazetidin-3-yl)oxy)methyl) pyridin-3-yl)-1H-imidazo[4,5-c]cinnolin-2(3H)-one
8-(6-((2-(3-azabicyclo[3.1.0]hexan-3-yl)propoxy)methyl)pyridin-3-yl)-1-isopropyl-3-methyl-1H-imidazo[4,5-c]cinnolin-2(3H)-one
8-(6-((2-(3-azabicyclo[3.1.0]hexan-3-yl)-2-methylpropoxy) methyl)pyridin-3-yl)-1-isopropyl-3-methyl-1H-imidazo[4,5-c]cinnolin-2(3H)-one
8-(6-(((1-(3-azabicyclo[3.1.0]hexan-3-yl)cyclopropyl) methoxy)methyl)pyridin-3-yl)-1-isopropyl-3-methyl-1H-imidazo[4,5-c]cinnolin-2(3H)-one
1-isopropyl-3-methyl-8-(6-(((1-methylpiperidin-3-yl)methoxy)methyl)pyridin-3-yl)-1H-imidazo[4,5-c]cinnolin-2(3H)-one
1-isopropyl-3-methyl-8-(6-(((1-methylpyrrolidin-3-yl)methoxy)methyl)pyridin-3-yl)-1H-imidazo[4,5-c]cinnolin-2(3H)-one
8-(6-(((1-(3-azabicyclo[3.1.0]hexan-3-yl)propan-2-yl)oxy)methyl)pyridin-3-yl)-1-isopropyl-3-methyl-1H-imidazo[4,5-c]cinnolin-2(3H)-one
8-(6-(((1-(3-azabicyclo[3.1.0]hexan-3-yl)-2-methylpropan-2-yl)oxy)methyl)pyridin-3-yl)-1-isopropyl-3-methyl-1H-imidazo[4,5-c]cinnolin-2(3H)-one
8-(6-((1-(3-azabicyclo[3.1.0]hexan-3-ylmethyl)cyclopropoxy) methyl)pyridin-3-yl)-1-isopropyl-3-methyl-1H-imidazo[4,5-c]cinnolin-2(3H)-one
1-(2-((5-(1-isopropyl-3-methyl-2-oxo-2,3-dihydro-1H-imidazo[4,5-c]cinnolin-8-yl)pyridin-2-yl)methoxy)ethyl)piperidine-4-carbonitrile
1-(2-((R)-1-(5-(1-isopropyl-3-methyl-2-oxo-2,3-dihydro-1H-imidazo[4,5-c]cinnolin-8-yl)pyridin-2-yl)ethoxy)ethyl)-3-methylpyrrolidine-3-carbonitrile
8-(6-((2-(5-azaspiro[2.4]heptan-5-yl)ethoxy)methyl)pyridin-3-yl)-1-isopropyl-3-methyl-1H-imidazo[4,5-c]cinnolin-2(3H)-one
1-isopropyl-8-(6-((2-(4-methoxypiperidin-1-yl)ethoxy)methyl) pyridin-3-yl)-3-methyl-1H-imidazo[4,5-c]cinnolin-2(3H)-one
(R)-8-(6-((2-(3-fluoropyrrolidin-1-yl)ethoxy)methyl)pyridin-3-yl)-1-isopropyl-3-methyl-1H-imidazo[4,5-c]cinnolin-2(3H)-one
(S)-1-isopropyl-3-methyl-8-(6-((2-(2-methylpyrrolidin-1-yl)ethoxy)methyl)pyridin-3-yl)-1H-imidazo[4,5-c]cinnolin-2(3H)-one
8-(6-((2-(4,4-difluoropiperidin-1-yl)ethoxy)methyl)pyridin-3-yl)-1-isopropyl-3-methyl-1H-imidazo[4,5-c]cinnolin-2(3H)-one
8-(6-((2-(4,4-dimethylpiperidin-1-yl)ethoxy)methyl)pyridin-3-yl)-1-isopropyl-3-methyl-1H-imidazo[4,5-c]cinnolin-2(3H)-one
1-isopropyl-3-methyl-8-(6-((2-(4-methylpiperazin-1-yl)ethoxy) methyl)pyridin-3-yl)-1H-imidazo[4,5-c]cinnolin-2(3H)-one
1-isopropyl-3-methyl-8-(6-((2-morpholinoethoxy)methyl) pyridin-3-yl)-1H-imidazo[4,5-c]cinnolin-2(3H)-one
(S)-8-(6-((2-(3-fluoropyrrolidin-1-yl)ethoxy)methyl)pyridin-3-yl)-1-isopropyl-3-methyl-1H-imidazo[4,5-c]cinnolin-2(3H)-one
(R)-1-isopropyl-3-methyl-8-(6-((2-(2-methylpyrrolidin-1-yl)ethoxy)methyl)pyridin-3-yl)-1H-imidazo[4,5-c]cinnolin-2(3H)-one
8-(6-((2-(3,3-dimethylpyrrolidin-1-yl)ethoxy)methyl)pyridin-3-yl)-1-isopropyl-3-methyl-1H-imidazo[4,5-c]cinnolin-2(3H)-one
8-(6-((2-(3-(dimethylamino)azetidin-1-yl)ethoxy)methyl)pyridin-3-yl)-1-isopropyl-3-methyl-1H-imidazo[4,5-c]cinnolin-2(3H)-one
8-(6-((2-(3-fluoropiperidin-1-yl)ethoxy)methyl)pyridin-3-yl)-1-isopropyl-3-methyl-1H-imidazo[4,5-c]cinnolin-2(3H)-one
8-(6-((2-(2-azabicyclo[2.2.1]heptan-2-yl)ethoxy)methyl)pyridin-3-yl)-1-isopropyl-3-methyl-1H-imidazo[4,5-c]cinnolin-2(3H)-one
8-(6-((2-(6-azaspiro[3.5]nonan-6-yl)ethoxy)methyl)pyridin-3-yl)-1-isopropyl-3-methyl-1H-imidazo[4,5-c]cinnolin-2(3H)-one
1-isopropyl-8-(6-((2-(3-methoxypiperidin-1-yl)ethoxy)methyl) pyridin-3-yl)-3-methyl-1H-imidazo[4,5-c]cinnolin-2(3H)-one
(S)-8-(6-((2-(3-(dimethylamino)pyrrolidin-1-yl)ethoxy)methyl) pyridin-3-yl)-1-isopropyl-3-methyl-1H-imidazo[4,5-c]cinnolin-2(3H)-one
(S)-1-isopropyl-8-(6-((2-(3-methoxypyrrolidin-1-yl)ethoxy)methyl)pyridin-3-yl)-3-methyl-1H-imidazo[4,5-c]cinnolin-2(3H)-one
1-isopropyl-8-(6-((2-(4-methoxypiperidin-1-yl)ethoxy)methyl) pyridin-3-yl)-3-methyl-1H-imidazo[4,5-c]cinnolin-2(3H)-one
8-(6-((2-((2R,5S)-2,5-dimethylpyrrolidin-1-yl)ethoxy)methyl) pyridin-3-yl)-1-isopropyl-3-methyl-1H-imidazo[4,5-c]cinnolin-2(3H)-one
(R)-8-(6-((2-(3-(dimethylamino)pyrrolidin-1-yl)ethoxy)methyl) pyridin-3-yl)-1-isopropyl-3-methyl-1H-imidazo[4,5-c]cinnolin-2(3H)-one
1-isopropyl-3-methyl-8-(6-((2-(4-(trifluoromethoxy)piperidin-1-yl)ethoxy)methyl)pyridin-3-yl)-1H-imidazo[4,5-c]cinnolin-2(3H)-one
8-(6-((2-(6,6-difluoro-3-azabicyclo[3.1.0]hexan-3-yl)ethoxy) methyl)pyridin-3-yl)-1-isopropyl-3-methyl-1H-imidazo[4,5-c]cinnolin-2(3H)-one
8-(6-((2-(7-azaspiro[3.5]nonan-7-yl)ethoxy)methyl)pyridin-3-yl)-1-isopropyl-3-methyl-1H-imidazo[4,5-c]cinnolin-2(3H)-one
(R)-8-(6-((2-(3-(difluoromethoxy)pyrrolidin-1-yl)ethoxy) methyl)pyridin-3-yl)-1-isopropyl-3-methyl-1H-imidazo[4,5-c]cinnolin-2(3H)-one
(S)-8-(6-((2-(3-(difluoromethoxy)pyrrolidin-1-yl)ethoxy) methyl)pyridin-3-yl)-1-isopropyl-3-methyl-1H-imidazo[4,5-c]cinnolin-2(3H)-one
1-isopropyl-3-methyl-8-(6-((2-(3-(trifluoromethoxy)pyrrolidin-1-yl)ethoxy)methyl)pyridin-3-yl)-1,3-dihydro-2H-imidazo[4,5-c]cinnolin-2-one
(R)-8-(6-(1-(2-(5-azaspiro[2.4]heptan-5-yl)ethoxy)ethyl)pyridin-3-yl)-1-isopropyl-3-methyl-1H-imidazo[4,5-c]cinnolin-2(3H)-one
(R)-8-(6-(1-(2-(6-azaspiro[2.5]octan-6-yl)ethoxy)ethyl)pyridin-3-yl)-1-isopropyl-3-methyl-1H-imidazo[4,5-c]cinnolin-2(3H)-one
(R)-8-(6-(1-(2-(6-azaspiro[2.5]octan-6-yl)ethoxy)ethyl)pyridin-3-yl)-1-isopropyl-3-methyl-1H-imidazo[4,5-c]cinnolin-2(3H)-one
(R)-1-isopropyl-8-(6-(1-(2-(4-methoxypiperidin-1-yl)ethoxy)ethyl)pyridin-3-yl)-3-methyl-1H-imidazo[4,5-c]cinnolin-2(3H)-one
1-isopropyl-8-(6-((R)-1-(2-((R)-3-methoxypyrrolidin-1-yl)ethoxy)ethyl)pyridin-3-yl)-3-methyl-1H-imidazo[4,5-c]cinnolin-2(3H)-one
(R)-1-isopropyl-3-methyl-8-(6-(1-(2-(4-(trifluoromethoxy)piperidin-1-yl)ethoxy)ethyl)pyridin-3-yl)-1H-imidazo[4,5-c]cinnolin-2(3H)-one
1-isopropyl-8-(6-((R)-1-(2-((S)-3-methoxypyrrolidin-1-yl)ethoxy)ethyl)pyridin-3-yl)-3-methyl-1H-imidazo[4,5-c]cinnolin-2(3H)-one
8-(6-((2-(3,3-dimethylpyrrolidin-1-yl)ethoxy)methyl)pyridin-3-yl)-7-fluoro-1-isopropyl-3-methyl-1H-imidazo[4,5-c]cinnolin-2(3H)-one
(R)-8-(6-(1-(2-(3,3-dimethylpyrrolidin-1-yl)ethoxy)ethyl)pyridin-3-yl)-1-isopropyl-3-methyl-1H-imidazo[4,5-c]cinnolin-2(3H)-one
(R)-1-isopropyl-3-methyl-8-(6-(1-(2-(pyrrolidin-1-yl)ethoxy)ethyl)pyridin-3-yl)-1,3-dihydro-2H-imidazo[4,5-c]cinnolin-2-one
8-(6-((R)-1-(2-((S)-3-(difluoromethoxy)pyrrolidin-1-yl)ethoxy)ethyl)pyridin-3-yl)-1-isopropyl-3-methyl-1H-imidazo[4,5-c]cinnolin-2(3H)-one
1-isopropyl-8-(6-((S)-1-(2-((R)-3-methoxypyrrolidin-1-yl)ethoxy)ethyl)pyridin-3-yl)-3-methyl-1H-imidazo[4,5-c]cinnolin-2(3H)-one
1-isopropyl-8-(6-((S)-1-(2-((S)-3-methoxypyrrolidin-1-yl)ethoxy)ethyl)pyridin-3-yl)-3-methyl-1H-imidazo[4,5-c]cinnolin-2(3H)-one
8-(6-((1S)-1-(2-(3-azabicyclo[3.1.0]hexan-3-yl)ethoxy)ethyl) pyridin-3-yl)-1-isopropyl-3-methyl-1H-imidazo[4,5-c]cinnolin-2(3H)-one
8-(6-((1R)-1-(2-(3-azabicyclo[3.1.0]hexan-3-yl)ethoxy)ethyl) pyridin-3-yl)-1-isopropyl-3-methyl-1H-imidazo[4,5-c]cinnolin-2(3H)-one
(S)-8-(6-(1-(2-(4-fluoropiperidin-1-yl)ethoxy)ethyl)pyridin-3-yl)-1-isopropyl-3-methyl-1H-imidazo[4,5-c]cinnolin-2(3H)-one
(R)-8-(6-(1-(2-(4-fluoropiperidin-1-yl)ethoxy)ethyl)pyridin-3-yl)-1-isopropyl-3-methyl-1H-imidazo[4,5-c]cinnolin-2(3H)-one
8-(6-((1S)-1-(2-(6,6-difluoro-3-azabicyclo[3.1.0]hexan-3-yl)ethoxy)ethyl)pyridin-3-yl)-1-isopropyl-3-methyl-1H-imidazo[4,5-c]cinnolin-2(3H)-one
8-(6-((1R)-1-(2-(6,6-difluoro-3-azabicyclo[3.1.0]hexan-3-yl)ethoxy)ethyl)pyridin-3-yl)-1-isopropyl-3-methyl-1H-imidazo[4,5-c]cinnolin-2(3H)-one
(S)-8-(6-(1-(2-(4,4-dimethylpiperidin-1-yl)ethoxy)ethyl)pyridin-3-yl)-1-isopropyl-3-methyl-1H-imidazo[4,5-c]cinnolin-2(3H)-one
(R)-8-(6-(1-(2-(4,4-dimethylpiperidin-1-yl)ethoxy)ethyl)pyridin-3-yl)-1-isopropyl-3-methyl-1H-imidazo[4,5-c]cinnolin-2(3H)-one
(S)-8-(6-(1-(2-(4,4-difluoropiperidin-1-yl)ethoxy)ethyl)pyridin-3-yl)-1-isopropyl-3-methyl-1H-imidazo[4,5-c]cinnolin-2(3H)-one
(R)-8-(6-(1-(2-(4,4-difluoropiperidin-1-yl)ethoxy)ethyl)pyridin-3-yl)-1-isopropyl-3-methyl-1H-imidazo[4,5-c]cinnolin-2(3H)-one
8-(6-((R)-1-(2-((R)-3-cyclopropoxypyrrolidin-1-yl)ethoxy)ethyl) pyridin-3-yl)-1-isopropyl-3-methyl-1H-imidazo[4,5-c]cinnolin-2(3H)-one
1-isopropyl-3-methyl-8-(6-((R)-1-(2-((R)-3-methylpyrrolidin-1-yl)ethoxy)ethyl)pyridin-3-yl)-1H-imidazo[4,5-c]cinnolin-2(3H)-one
1-isopropyl-3-methyl-8-(6-((R)-1-(2-((S)-3-methylpyrrolidin-1-yl)ethoxy)ethyl)pyridin-3-yl)-1H-imidazo[4,5-c]cinnolin-2(3H)-one
1-isopropyl-3-methyl-8-(6-((R)-1-(2-((R)-3-(methylsulfonyl)pyrrolidin-1-yl)ethoxy)ethyl)pyridin-3-yl)-1H-imidazo[4,5-c]cinnolin-2(3H)-one
8-(6-((R)-1-(2-((R)-3-cyclopropoxypyrrolidin-1-yl)ethoxy)ethyl) pyridin-3-yl)-3-methyl-1-(tetrahydro-2H-pyran-4-yl)-1H-imidazo[4,5-c]cinnolin-2(3H)-one
(R)-8-(6-(1-(2-(2-oxa-6-azaspiro[3.3]heptan-6-yl)ethoxy)ethyl) pyridin-3-yl)-1-isopropyl-3-methyl-1H-imidazo[4,5-c]cinnolin-2(3H)-one
(R)-8-(6-(1-(2-(1-oxa-7-azaspiro[3.5]nonan-7-yl)ethoxy)ethyl) pyridin-3-yl)-1-isopropyl-3-methyl-1H-imidazo[4,5-c]cinnolin-2(3H)-one
(R)-1-isopropyl-3-methyl-8-(6-(1-(2-(4-(oxetan-3-yl)piperidin-1-yl)ethoxy)ethyl)pyridin-3-yl)-1H-imidazo[4,5-c]cinnolin-2(3H)-one
(R)-1-isopropyl-3-methyl-8-(6-(1-(2-(4-(trifluoromethyl) piperidin-1-yl)ethoxy)ethyl)pyridin-3-yl)-1H-imidazo[4,5-c]cinnolin-2(3H)-one
1-isopropyl-8-(6-((R)-1-(2-((R)-3-(methoxymethyl)pyrrolidin-1-yl)ethoxy)ethyl)pyridin-3-yl)-3-methyl-1H-imidazo[4,5-c]cinnolin-2(3H)-one
(R)-8-(6-(1-(2-(2-oxa-6-azaspiro[3.4]octan-6-yl)ethoxy)ethyl) pyridin-3-yl)-1-isopropyl-3-methyl-1H-imidazo[4,5-c]cinnolin-2(3H)-one
(R)-1-isopropyl-3-methyl-8-(6-(1-(2-(piperidin-1-yl)ethoxy)ethyl) pyridin-3-yl)-1H-imidazo[4,5-c]cinnolin-2(3H)-one
1-isopropyl-3-methyl-8-(6-((R)-1-(2-((R)-3-(trifluoromethoxy) pyrrolidin-1-yl)ethoxy)ethyl)pyridin-3-yl)-1H-imidazo[4,5-c]cinnolin-2(3H)-one
(R)-8-(6-(1-(2-(6-azaspiro[3.4]octan-6-yl)ethoxy)ethyl)pyridin-3-yl)-1-isopropyl-3-methyl-1H-imidazo[4,5-c]cinnolin-2(3H)-one
1-(2-((R)-1-(5-(1-isopropyl-3-methyl-2-oxo-2,3-dihydro-1H-imidazo[4,5-c]cinnolin-8-yl)pyridin-2-yl)ethoxy)ethyl)-3-methylpyrrolidine-3-carbonitrile
(R)-1-(2-(1-(5-(1-isopropyl-3-methyl-2-oxo-2,3-dihydro-1H-imidazo[4,5-c]cinnolin-8-yl)pyridin-2-yl)ethoxy)ethyl)piperidine-4-carbonitrile
(R)-8-(6-(1-(2-(4-isopropoxypiperidin-1-yl)ethoxy)ethyl)pyridin-3-yl)-1-isopropyl-3-methyl-1H-imidazo[4,5-c]cinnolin-2(3H)-one
8-(6-((R)-1-(2-((R)-3-isopropoxypyrrolidine-1-yl)ethoxy) ethyl)pyridin-3-yl)-1-isopropyl-3-methyl-1H-imidazo[4,5-c]cinnolin-2(3H)-one
8-(6-((2-(5-azaspiro[2.4]heptan-5-yl)ethoxy)methyl)pyridin-3-yl)-3-methyl-1-(tetrahydro-2H-pyran-4-yl)-1H-imidazo[4,5-c]cinnolin-2(3H)-one
8-(6-((2-(4-fluoropiperidin-1-yl)ethoxy)methyl)pyridin-3-yl)-3-methyl-1-(tetrahydro-2H-pyran-4-yl)-1H-imidazo[4,5-c]cinnolin-2(3H)-one
8-(6-((2-(4,4-dimethylpiperidin-1-yl)ethoxy)methyl)pyridin-3-yl)-3-methyl-1-(tetrahydro-2H-pyran-4-yl)-1H-imidazo[4,5-c]cinnolin-2(3H)-one
8-(6-((2-(3,3-dimethylpyrrolidin-1-yl)ethoxy)methyl)pyridin-3-yl)-3-methyl-1-(tetrahydro-2H-pyran-4-yl)-1H-imidazo[4,5-c]cinnolin-2(3H)-one
8-(6-((2-(3-azabicyclo[3.1.0]hexan-3-yl)ethoxy)methyl)pyridin-3-yl)-3-methyl-1-(tetrahydro-2H-pyran-4-yl)-1H-imidazo[4,5-c]cinnolin-2(3H)-one
(R)-8-(6-((2-(3-(difluoromethoxy)pyrrolidin-1-yl)ethoxy)methyl) pyridin-3-yl)-3-methyl-1-(tetrahydro-2H-pyran-4-yl)-1H-imidazo[4,5-c]cinnolin-2(3H)-one
(S)-8-(6-((2-(3-(difluoromethoxy)pyrrolidin-1-yl)ethoxy)methyl)pyridin-3-yl)-3-methyl-1-(tetrahydro-2H-pyran-4-yl)-1H-imidazo[4,5-c]cinnolin-2(3H)-one
3-methyl-1-(tetrahydro-2H-pyran-4-yl)-8-(6-((2-(4-(trifluoromethoxy)piperidin-1-yl)ethoxy)methyl)pyridin-3-yl)-1H-imidazo[4,5-c]cinnolin-2(3H)-one
(R)-8-(6-(1-(2-(5-azaspiro[2.4]heptan-5-yl)ethoxy)ethyl)pyridin-3-yl)-3-methyl-1-(tetrahydro-2H-pyran-4-yl)-1H-imidazo[4,5-c]cinnolin-2(3H)-one
(R)-8-(6-(1-(2-(4,4-dimethylpiperidin-1-yl)ethoxy)ethyl)pyridin-3-yl)-3-methyl-1-(tetrahydro-2H-pyran-4-yl)-1H-imidazo[4,5-c]cinnolin-2(3H)-one
8-(6-((1R)-1-(2-(3-azabicyclo[3.1.0]hexan-3-yl)ethoxy)ethyl)pyridin-3-yl)-3-methyl-1-(tetrahydro-2H-pyran-4-yl)-1H-imidazo[4,5-c]cinnolin-2(3H)-one
8-(6-((R)-1-(2-((S)-3-methoxypyrrolidin-1-yl)ethoxy)ethyl)pyridin-3-yl)-3-methyl-1-(tetrahydro-2H-pyran-4-yl)-1H-imidazo[4,5-c]cinnolin-2(3H)-one
3-methyl-1-(tetrahydro-2H-pyran-4-yl)-8-(6-((1R)-1-(2-(3-(trifluoromethoxy)pyrrolidin-1-yl)ethoxy)ethyl)pyridin-3-yl)-1H-imidazo[4,5-c]cinnolin-2(3H)-one
(R)-8-(6-(1-(2-(6-azaspiro[2.5]octan-6-yl)ethoxy)ethyl)pyridin-3-yl)-3-methyl-1-(tetrahydro-2H-pyran-4-yl)-1H-imidazo[4,5-c]cinnolin-2(3H)-one
8-(6-((R)-1-(2-(4-fluoropiperidin-1-yl)ethoxy)ethyl)pyridin-3-yl)-3-methyl-1-(tetrahydro-2H-pyran-4-yl)-1,3,3a,9b-tetrahydro-2H-imidazo[4,5-c]cinnolin-2-one (R)-3-methyl-1-(tetrahydro-2H-pyran-4-yl)-8-(6-(1-(2-(4-(trifluoromethoxy)piperidin-1-yl)ethoxy)ethyl)pyridin-3-yl)-1H-imidazo[4,5-c]cinnolin-2(3H)-one
(R)-8-(6-(1-(2-(4-methoxypiperidin-1-yl)ethoxy)ethyl)pyridin-3-yl)-3-methyl-1-(tetrahydro-2H-pyran-4-yl)-1H-imidazo[4,5-c]cinnolin-2(3H)-one
(R)-8-(6-(1-(2-(3,3-dimethylpyrrolidin-1-yl)ethoxy)ethyl)pyridin-3-yl)-3-methyl-1-(tetrahydro-2H-pyran-4-yl)-1H-imidazo[4,5-c]cinnolin-2(3H)-one
8-(6-((R)-1-(2-((R)-3-methoxypyrrolidin-1-yl)ethoxy)ethyl) pyridin-3-yl)-3-methyl-1-(tetrahydro-2H-pyran-4-yl)-1H-imidazo[4,5-c]cinnolin-2(3H)-one
8-(6-((R)-1-(2-((R)-3-isopropoxypyrrolidin-1-yl)ethoxy)ethyl)pyridin-3-yl)-3-methyl-1-(tetrahydro-2H-pyran-4-yl)-1H-imidazo[4,5-c]cinnolin-2(3H)-one
(R)-1-(2-(1-(5-(3-methyl-2-oxo-1-(tetrahydro-2H-pyran-4-yl)-2,3-dihydro-1H-imidazo[4,5-c]cinnolin-8-yl)pyridin-2-yl)ethoxy)ethyl)piperidine-4-carbonitrile
8-(6-((R)-1-(2-((S)-3-(difluoromethoxy)pyrrolidin-1-yl)ethoxy)ethyl)pyridin-3-yl)-3-methyl-1-(tetrahydro-2H-pyran-4-yl)-1,3-dihydro-2H-imidazo[4,5-c]cinnolin-2-one
(R)-8-(6-(1-(2-(2-oxa-6-azaspiro[3.4]octan-6-yl)ethoxy)ethyl) pyridin-3-yl)-3-methyl-1-(tetrahydro-2H-pyran-4-yl)-1H-imidazo[4,5-c]cinnolin-2(3H)-one
(R)-3-methyl-1-(tetrahydro-2H-pyran-4-yl)-8-(6-(1-(2-(4-(trifluoromethyl)piperidin-1-yl)ethoxy)ethyl)pyridin-3-yl)-1H-imidazo[4,5-c]cinnolin-2(3H)-one
(R)-8-(6-(1-(2-(6-azaspiro[3.4]octan-6-yl)ethoxy)ethyl)pyridin-3-yl)-3-methyl-1-(tetrahydro-2H-pyran-4-yl)-1H-imidazo[4,5-c]cinnolin-2(3H)-one
8-(6-((R)-1-(2-((R)-3-(methoxymethyl)pyrrolidin-1-yl)ethoxy) ethyl)pyridin-3-yl)-3-methyl-1-(tetrahydro-2H-pyran-4-yl)-1H-imidazo[4,5-c]cinnolin-2(3H)-one
(R)-3-methyl-8-(6-(1-(2-(piperidin-1-yl)ethoxy)ethyl)pyridin-3-yl)-1-(tetrahydro-2H-pyran-4-yl)-1H-imidazo[4,5-c]cinnolin-2(3H)-one
(R)-3-methyl-8-(6-(1-(2-(pyrrolidin-1-yl)ethoxy)ethyl)pyridin-3-yl)-1-(tetrahydro-2H-pyran-4-yl)-1H-imidazo[4,5-c]cinnolin-2(3H)-one
8-(6-((R)-1-(2-((R)-3-cyclopropoxypyrrolidin-1-yl)ethoxy)ethyl) pyridin-3-yl)-3-methyl-1-(tetrahydro-2H-pyran-4-yl)-1H-imidazo[4,5-c]cinnolin-2(3H)-one
3-methyl-8-(6-((R)-1-(2-((R)-3-methylpyrrolidin-1-yl)ethoxy)ethyl)pyridin-3-yl)-1-(tetrahydro-2H-pyran-4-yl)-1H-imidazo[4,5-c]cinnolin-2(3H)-one
(R)-8-(6-(1-(2-(7-azaspiro[3.5]nonan-7-yl)ethoxy)ethyl)pyridin-3-yl)-3-methyl-1-(tetrahydro-2H-pyran-4-yl)-1H-imidazo[4,5-c]cinnolin-2(3H)-one
(R)-8-(6-(1-(2-(I-oxa-7-azaspiro[3.5]nonan-7-yl)ethoxy)ethyl)pyridin-3-yl)-3-methyl-1-(tetrahydro-2H-pyran-4-yl)-1H-imidazo[4,5-c]cinnolin-2(3H)-one
3-methyl-8-(6-((R)-1-(2-((S)-3-methylpyrrolidin-1-yl)ethoxy)ethyl)pyridin-3-yl)-1-(tetrahydro-2H-pyran-4-yl)-1H-imidazo[4,5-c]cinnolin-2(3H)-one
(R)-8-(6-(1-(2-(2-oxa-6-azaspiro[3.3]heptan-6-yl)ethoxy)ethyl)pyridin-3-yl)-3-methyl-1-(tetrahydro-2H-pyran-4-yl)-1H-imidazo[4,5-c]cinnolin-2(3H)-one
(R)-3-methyl-8-(6-(1-(2-(4-(oxetan-3-yl)piperidin-1-yl)ethoxy)ethyl)pyridin-3-yl)-1-(tetrahydro-2H-pyran-4-yl)-1H-imidazo[4,5-c]cinnolin-2(3H)-one
3-methyl-8-(6-((R)-1-(2-((R)-3-(methylsulfonyl)pyrrolidin-1-yl)ethoxy)ethyl)pyridin-3-yl)-1-(tetrahydro-2H-pyran-4-yl)-1H-imidazo[4,5-c]cinnolin-2(3H)-one
3-methyl-1-(2-((R)-1-(5-(3-methyl-2-oxo-1-(tetrahydro-2H-pyran-4-yl)-2,3-dihydro-1H-imidazo[4,5-c]cinnolin-8-yl)pyridin-2-yl)ethoxy)ethyl) pyrrolidine-3-carbonitrile
8-(6-((2-(5-azaspiro[2.4]heptan-5-yl)ethoxy)methyl)pyridin-3-yl)-7-fluoro-1-isopropyl-3-methyl-1H-imidazo[4,5-c]cinnolin-2(3H)-one
8-(6-((2-(3,3-dimethylpyrrolidin-1-yl)ethoxy)methyl)pyridin-3-yl)-7-fluoro-1-isopropyl-3-methyl-1H-imidazo[4,5-c]cinnolin-2(3H)-one
8-(6-((2-(4,4-dimethylpiperidin-1-yl)ethoxy)methyl)pyridin-3-yl)-7-fluoro-1-isopropyl-3-methyl-1H-imidazo[4,5-c]cinnolin-2(3H)-one
7-fluoro-1-isopropyl-3-methyl-8-(6-((2-(3-(trifluoromethoxy)pyrrolidin-1-yl)ethoxy)methyl)pyridin-3-yl)-1,3-dihydro-2H-imidazo[4,5-c]cinnolin-2-one
(R)-8-(6-(1-(2-(5-azaspiro[2.4]heptan-5-yl)ethoxy)ethyl)pyridin-3-yl)-7-fluoro-3-methyl-1-(tetrahydro-2H-pyran-4-yl)-1H-imidazo[4,5-c]cinnolin-2(3H)-one
(R)-7-fluoro-8-(6-(1-(2-(4-fluoropiperidin-1-yl)ethoxy)ethyl)pyridin-3-yl)-1-isopropyl-3-methyl-1,3-dihydro-2H-imidazo[4,5-c]cinnolin-2-one
(R)-8-(6-(1-(2-(3,3-dimethylpyrrolidin-1-yl)ethoxy)ethyl)pyridin-3-yl)-7-fluoro-1-isopropyl-3-methyl-1H-imidazo[4,5-c]cinnolin-2(3H)-one
7-fluoro-1-isopropyl-8-(6-((R)-1-(2-((S)-3-methoxypyrrolidin-1-yl)ethoxy)ethyl)pyridin-3-yl)-3-methyl-1H-imidazo[4,5-c]cinnolin-2(3H)-one
(R)-8-(6-(1-(2-(4,4-dimethylpiperidin-1-yl)ethoxy)ethyl)pyridin-3-yl)-7-fluoro-1-isopropyl-3-methyl-1H-imidazo[4,5-c]cinnolin-2(3H)-one
(R)-8-(6-(1-(2-(6-azaspiro[2.5]octan-6-yl)ethoxy)ethyl)pyridin-3-yl)-7-fluoro-1-isopropyl-3-methyl-1H-imidazo[4,5-c]cinnolin-2(3H)-one
7-fluoro-1-isopropyl-3-methyl-8-(6-((1R)-1-(2-(3-(trifluoromethoxy)pyrrolidin-1-yl)ethoxy)ethyl)pyridin-3-yl)-1H-imidazo[4,5-c]cinnolin-2(3H)-one
(R)-7-fluoro-1-isopropyl-8-(6-(1-(2-(4-methoxypiperidin-1-yl)ethoxy)ethyl)pyridin-3-yl)-3-methyl-1H-imidazo[4,5-c]cinnolin-2(3H)-one
8-(6-((1R)-1-(2-(3-azabicyclo[3.1.0]hexan-3-yl)ethoxy)ethyl)pyridin-3-yl)-7-fluoro-1-isopropyl-3-methyl-1H-imidazo[4,5-c]cinnolin-2(3H)-one
(R)-7-fluoro-1-isopropyl-3-methyl-8-(6-(1-(2-(4-(trifluoromethoxy)piperidin-1-yl)ethoxy)ethyl)pyridin-3-yl)-1H-imidazo[4,5-c]cinnolin-2(3H)-one
(R)-1-(2-(1-(5-(7-fluoro-1-isopropyl-3-methyl-2-oxo-2,3-dihydro-1H-imidazo[4,5-c]cinnolin-8-yl)pyridin-2-yl)ethoxy)ethyl)piperidine-4-carbonitrile
8-(6-((R)-1-(2-((R)-3-(difluoromethoxy)pyrrolidin-1-yl)ethoxy)ethyl)pyridin-3-yl)-7-fluoro-1-isopropyl-3-methyl-1H-imidazo[4,5-c]cinnolin-2(3H)-one
1-(2-((R)-1-(5-(7-fluoro-1-isopropyl-3-methyl-2-oxo-2,3-dihydro-1H-imidazo[4,5-c]cinnolin-8-yl)pyridin-2-yl)ethoxy)ethyl)-3-methylpyrrolidine-3-carbonitrile
(R)-8-(6-(1-(2-(5-azaspiro[2.4]heptan-5-yl)ethoxy)ethyl)pyridin-3-yl)-7-fluoro-3-methyl-1-(tetrahydro-2H-pyran-4-yl)-1H-imidazo[4,5-c]cinnolin-2(3H)-one
(R)-8-(6-(1-(2-(4,4-dimethylpiperidin-1-yl)ethoxy)ethyl)pyridin-3-yl)-7-fluoro-3-methyl-1-(tetrahydro-2H-pyran-4-yl)-1H-imidazo[4,5-c]cinnolin-2(3H)-one
(R)-7-fluoro-8-(6-(1-(2-(4-fluoropiperidin-1-yl)ethoxy)ethyl)pyridin-3-yl)-3-methyl-1-(tetrahydro-2H-pyran-4-yl)-1H-imidazo[4,5-c]cinnolin-2(3H)-one
7-fluoro-3-methyl-1-(tetrahydro-2H-pyran-4-yl)-8-(6-((R)-1-(2-((R)-3-(trifluoromethoxy)pyrrolidin-1-yl)ethoxy)ethyl)pyridin-3-yl)-1,3-dihydro-2H-imidazo[4,5-c]cinnolin-2-one
7-fluoro-3-methyl-1-(tetrahydro-2H-pyran-4-yl)-8-(6-((R)-1-(2-((S)-3-(trifluoromethoxy)pyrrolidin-1-yl)ethoxy)ethyl)pyridin-3-yl)-1,3-dihydro-2H-imidazo[4,5-c]cinnolin-2-one
(R)-8-(6-(1-(2-(3,3-dimethylpyrrolidin-1-yl)ethoxy)ethyl)pyridin-3-yl)-7-fluoro-3-methyl-1-(tetrahydro-2H-pyran-4-yl)-1H-imidazo[4,5-c]cinnolin-2(3H)-one
(R)-8-(6-(1-(2-(6-azaspiro[2.5]octan-6-yl)ethoxy)ethyl)pyridin-3-yl)-7-fluoro-3-methyl-1-(tetrahydro-2H-pyran-4-yl)-1H-imidazo[4,5-c]cinnolin-2(3H)-one
8-(6-((R)-1-(2-((R)-3-(difluoromethoxy)pyrrolidin-1-yl)ethoxy)ethyl)pyridin-3-yl)-7-fluoro-3-methyl-1-(tetrahydro-2H-pyran-4-yl)-1H-imidazo[4,5-c]cinnolin-2(3H)-one
(R)-8-(6-(1-(2-(7-azaspiro[3.5]nonan-7-yl)ethoxy)ethyl)pyridin-3-yl)-7-fluoro-3-methyl-1-(tetrahydro-2H-pyran-4-yl)-1H-imidazo[4,5-c]cinnolin-2(3H)-one
(R)-7-fluoro-3-methyl-1-(tetrahydro-2H-pyran-4-yl)-8-(6-(1-(2-(4-(trifluoromethoxy)piperidin-1-yl)ethoxy)ethyl)pyridin-3-yl)-1H-imidazo[4,5-c]cinnolin-2(3H)-one
8-(6-((R)-1-(2-((S)-3-(difluoromethoxy)pyrrolidin-1-yl)ethoxy)ethyl)pyridin-3-yl)-7-fluoro-3-methyl-1-(tetrahydro-2H-pyran-4-yl)-1,3-dihydro-2H-imidazo[4,5-c]cinnolin-2-one 8-(6-((2-(5-azaspiro[2.4]heptan-5-yl)ethoxy)methyl)pyridin-3-yl)-1-(trans-3-methoxycyclobutyl)-3-methyl-1H-imidazo[4,5-c]cinnolin-2(3H)-one
8-(6-((2-(3,3-dimethylpyrrolidin-1-yl)ethoxy)methyl)pyridin-3-yl)-1-(trans-3-methoxycyclobutyl)-3-methyl-1H-imidazo[4,5-c]cinnolin-2(3H)-one
8-(6-((R)-1-(2-(5-azaspiro[2.4]heptan-5-yl)ethoxy)ethyl)pyridin-3-yl)-1-(trans-3-methoxycyclobutyl)-3-methyl-1H-imidazo[4,5-c]cinnolin-2(3H)-one
1-(trans-3-methoxycyclobutyl)-3-methyl-8-(6-((R)-1-(2-(pyrrolidin-1-yl)ethoxy)ethyl)pyridin-3-yl)-1,3-dihydro-2H-imidazo[4,5-c]cinnolin-2-one
1-(trans-3-methoxycyclobutyl)-8-(6-((R)-1-(2-((R)-3-methoxypyrrolidin-1-yl)ethoxy)ethyl)pyridin-3-yl)-3-methyl-1H-imidazo[4,5-c]cinnolin-2(3H)-one
8-(6-((R)-1-(2-(6-azaspiro[2.5]octan-6-yl)ethoxy)ethyl)pyridin-3-yl)-1-(trans-3-methoxycyclobutyl)-3-methyl-1H-imidazo[4,5-c]cinnolin-2(3H)-one
8-(6-((2-(dimethylamino)ethoxy)methyl)pyridin-3-yl)-1-(trans-3-methoxycyclobutyl)-3-methyl-1H-imidazo[4,5-c]cinnolin-2(3H)-one
1-(trans-3-methoxycyclobutyl)-3-methyl-8-(6-((2-(pyrrolidin-1-yl)ethoxy)methyl)pyridin-3-yl)-1H-imidazo[4,5-c]cinnolin-2(3H)-one
1-(trans-3-methoxycyclobutyl)-3-methyl-8-(6-((2-(piperidin-1-yl)ethoxy)methyl)pyridin-3-yl)-1H-imidazo[4,5-c]cinnolin-2(3H)-one
8-(6-((2-(4-fluoropiperidin-1-yl)ethoxy)methyl)pyridin-3-yl)-1-(trans-3-methoxycyclobutyl)-3-methyl-1H-imidazo[4,5-c]cinnolin-2(3H)-one
8-(6-((2-(3-azabicyclo[3.1.0]hexan-3-yl)ethoxy)methyl)pyridin-3-yl)-1-(trans-3-methoxycyclobutyl)-3-methyl-1H-imidazo[4,5-c]cinnolin-2(3H)-one
8-(6-((R)-1-(2-(6-azaspiro[2.5]octan-6-yl)ethoxy)ethyl)pyridin-3-yl)-1-(cis-3-methoxycyclobutyl)-3-methyl-1H-imidazo[4,5-c]cinnolin-2(3H)-one
8-(6-((R)-1-(2-(5-azaspiro[2.4]heptan-5-yl)ethoxy)ethyl)pyridin-3-yl)-1-(cis-3-methoxycyclobutyl)-3-methyl-1H-imidazo[4,5-c]cinnolin-2(3H)-one
1-(cis-3-methoxycyclobutyl)-3-methyl-8-(6-((2-(pyrrolidin-1-yl)ethoxy)methyl)pyridin-3-yl)-1H-imidazo[4,5-c]cinnolin-2(3H)-one
1-(cis-3-methoxycyclobutyl)-3-methyl-8-(6-((2-(piperidin-1-yl)ethoxy)methyl)pyridin-3-yl)-1H-imidazo[4,5-c]cinnolin-2(3H)-one
8-(6-((2-(4-fluoropiperidin-1-yl)ethoxy)methyl)pyridin-3-yl)-1-(cis-3-methoxycyclobutyl)-3-methyl-1H-imidazo[4,5-c]cinnolin-2(3H)-one
8-(6-((2-(3-azabicyclo[3.1.0]hexan-3-yl)ethoxy)methyl)pyridin-3-yl)-1-(cis-3-methoxycyclobutyl)-3-methyl-1H-imidazo[4,5-c]cinnolin-2(3H)-one
8-(6-((2-(dimethylamino)ethoxy)methyl)pyridin-3-yl)-1-(cis-3-methoxycyclobutyl)-3-methyl-1H-imidazo[4,5-c]cinnolin-2(3H)-one
8-(6-((2-(dimethylamino)ethoxy)methyl)pyridin-3-yl)-3-methyl-1-(oxetan-3-yl)-1H-imidazo[4,5-c]cinnolin-2(3H)-one
3-methyl-1-(oxetan-3-yl)-8-(6-((2-(piperidin-1-yl)ethoxy)methyl)pyridin-3-yl)-1H-imidazo[4,5-c]cinnolin-2(3H)-one
8-(6-((2-(4-fluoropiperidin-1-yl)ethoxy)methyl)pyridin-3-yl)-3-methyl-1-(oxetan-3-yl)-1H-imidazo[4,5-c]cinnolin-2(3H)-one
8-(6-((2-(4-fluoropiperidin-1-yl)ethoxy)methyl)pyridin-3-yl)-1-((1s,3s)-3-methoxycyclobutyl)-3-methyl-1H-imidazo[4,5-c]cinnolin-2(3H)-one
3-methyl-1-(oxetan-3-yl)-8-(6-((2-(pyrrolidin-1-yl)ethoxy) methyl)pyridin-3-yl)-1H-imidazo[4,5-c]cinnolin-2(3H)-one
(S)-8-(6-((2-(4-fluoropiperidin-1-yl)ethoxy)methyl)pyridin-3-yl)-3-methyl-1-(tetrahydro-2H-pyran-3-yl)-1H-imidazo[4,5-c]cinnolin-2(3H)-one
8-(6-((2-(3-azabicyclo[3.1.0]hexan-3-yl)ethoxy)methyl)pyridin-3-yl)-3-methyl-1-((S)-tetrahydro-2H-pyran-3-yl)-1H-imidazo[4,5-c]cinnolin-2(3H)-one
(S)-1-isopropyl-3-methyl-8-(6-(2,2,2-trifluoro-1-(2-(pyrrolidin-1-yl)ethoxy)ethyl)pyridin-3-yl)-1,3-dihydro-2H-imidazo[4,5-c]cinnolin-2-one
(R)-1-isopropyl-3-methyl-8-(6-(2,2,2-trifluoro-1-(2-(pyrrolidin-1-yl)ethoxy)ethyl)pyridin-3-yl)-1,3-dihydro-2H-imidazo[4,5-c]cinnolin-2-one
1-isopropyl-3-methyl-8-(6-(2,2,2-trifluoro-1-(2-(pyrrolidin-1-yl)ethoxy)ethyl)pyridin-3-yl)-1,3-dihydro-2H-imidazo[4,5-c]cinnolin-2-one
1-isopropyl-3-methyl-8-(6-(2,2,2-trifluoro-1-(2-(3-methylpyrrolidin-1-yl)ethoxy)ethyl)pyridin-3-yl)-1,3-dihydro-2H-imidazo[4,5-c]cinnolin-2-one
1-isopropyl-3-methyl-8-(6-(2,2,2-trifluoro-1-(2-(3-methoxypyrrolidin-1-yl)ethoxy)ethyl)pyridin-3-yl)-1,3-dihydro-2H-imidazo[4,5-c]cinnolin-2-one
8-(6-(1-(2-(3,3-dimethylpyrrolidin-1-yl)ethoxy)-2,2,2-trifluoroethyl)pyridin-3-yl)-1-isopropyl-3-methyl-1,3-dihydro-2H-imidazo[4,5-c]cinnolin-2-one
8-(6-(1-(2-(5-azaspiro[2.4]heptan-5-yl)ethoxy)-2,2,2-trifluoroethyl)pyridin-3-yl)-1-isopropyl-3-methyl-1,3-dihydro-2H-imidazo[4,5-c]cinnolin-2-one
8-(6-(1-(2-(3-azabicyclo[3.1.0]hexan-3-yl)ethoxy)-2,2,2-trifluoroethyl)pyridin-3-yl)-1-isopropyl-3-methyl-1,3-dihydro-2H-imidazo[4,5-c]cinnolin-2-one
1-isopropyl-3-methyl-8-(6-(2,2,2-trifluoro-1-(2-(piperidin-1-yl)ethoxy)ethyl)pyridin-3-yl)-1,3-dihydro-2H-imidazo[4,5-c]cinnolin-2-one
8-(6-(1-(2-(4,4-dimethylpiperidin-1-yl)ethoxy)-2,2,2-trifluoroethyl)pyridin-3-yl)-1-isopropyl-3-methyl-1,3-dihydro-2H-imidazo[4,5-c]cinnolin-2-one
1-isopropyl-3-methyl-8-(6-(2,2,2-trifluoro-1-(2-(4-methoxypiperidin-1-yl)ethoxy)ethyl)pyridin-3-yl)-1,3-dihydro-2H-imidazo[4,5-c]cinnolin-2-one
8-(6-(1-(2-(6-azaspiro[2.5]octan-6-yl)ethoxy)-2,2,2-trifluoroethyl)pyridin-3-yl)-1-isopropyl-3-methyl-1,3-dihydro-2H-imidazo[4,5-c]cinnolin-2-one
3-methyl-1-(tetrahydro-2H-pyran-4-yl)-8-(6-(2,2,2-trifluoro-1-(2-(pyrrolidin-1-yl)ethoxy)ethyl)pyridin-3-yl)-1,3-dihydro-2H-imidazo[4,5-c]cinnolin-2-one
3-methyl-1-(tetrahydro-2H-pyran-4-yl)-8-(6-(2,2,2-trifluoro-1-(2-(3-methylpyrrolidin-1-yl)ethoxy)ethyl)pyridin-3-yl)-1,3-dihydro-2H-imidazo[4,5-c]cinnolin-2-one
3-methyl-1-(tetrahydro-2H-pyran-4-yl)-8-(6-(2,2,2-trifluoro-1-(2-(3-methoxypyrrolidin-1-yl)ethoxy)ethyl)pyridin-3-yl)-1,3-dihydro-2H-imidazo[4,5-c]cinnolin-2-one
8-(6-(1-(2-(3,3-dimethylpyrrolidin-1-yl)ethoxy)-2,2,2-trifluoroethyl)pyridin-3-yl)-3-methyl-1-(tetrahydro-2H-pyran-4-yl)-1,3-dihydro-2H-imidazo[4,5-c]cinnolin-2-one
8-(6-(1-(2-(5-azaspiro[2.4]heptan-5-yl)ethoxy)-2,2,2-trifluoroethyl)pyridin-3-yl)-3-methyl-1-(tetrahydro-2H-pyran-4-yl)-1,3-dihydro-2H-imidazo[4,5-c]cinnolin-2-one
8-(6-(1-(2-(3-azabicyclo[3.1.0]hexan-3-yl)ethoxy)-2,2,2-trifluoroethyl)pyridin-3-yl)-3-methyl-1-(tetrahydro-2H-pyran-4-yl)-1,3-dihydro-2H-imidazo[4,5-c]cinnolin-2-one
3-methyl-1-(tetrahydro-2H-pyran-4-yl)-8-(6-(2,2,2-trifluoro-1-(2-(piperidin-1-yl)ethoxy)ethyl)pyridin-3-yl)-1,3-dihydro-2H-imidazo[4,5-c]cinnolin-2-one
8-(6-(1-(2-(4,4-dimethylpiperidin-1-yl)ethoxy)-2,2,2-trifluoroethyl)pyridin-3-yl)-3-methyl-1-(tetrahydro-2H-pyran-4-yl)-1,3-dihydro-2H-imidazo[4,5-c]cinnolin-2-one
3-methyl-1-(tetrahydro-2H-pyran-4-yl)-8-(6-(2,2,2-trifluoro-1-(2-(4-methoxypiperidin-1-yl)ethoxy)ethyl)pyridin-3-yl)-1,3-dihydro-2H-imidazo[4,5-c]cinnolin-2-one
8-(6-(1-(2-(6-azaspiro[2.5]octan-6-yl)ethoxy)-2,2,2-trifluoroethyl)pyridin-3-yl)-3-methyl-1-(tetrahydro-2H-pyran-4-yl)-1,3-dihydro-2H-imidazo[4,5-c]cinnolin-2-one
1-(3-methoxycyclobutyl)-3-methyl-8-(6-(2,2,2-trifluoro-1-(2-(pyrrolidin-1-yl)ethoxy)ethyl)pyridin-3-yl)-1,3-dihydro-2H-imidazo[4,5-c]cinnolin-2-one
1-(3-methoxycyclobutyl)-3-methyl-8-(6-(2,2,2-trifluoro-1-(2-(3-methylpyrrolidin-1-yl)ethoxy)ethyl)pyridin-3-yl)-1,3-dihydro-2H-imidazo[4,5-c]cinnolin-2-one
1-(3-methoxycyclobutyl)-3-methyl-8-(6-(2,2,2-trifluoro-1-(2-(3-methoxypyrrolidin-1-yl)ethoxy)ethyl)pyridin-3-yl)-1,3-dihydro-2H-imidazo[4,5-c]cinnolin-2-one
8-(6-(1-(2(3,3-dimethylpyrrolidin-1-yl)ethoxy)-2,2,2-trifluoroethyl)pyridin-3-yl)-1-(3-methoxycyclobutyl)-3-methyl-1,3-dihydro-2H-imidazo[4,5-c]cinnolin-2-one
8-(6-(1-(2-(5-azaspiro[2.4]heptan-5-yl)ethoxy)-2,2,2-trifluoroethyl)pyridin-3-yl)-1-(3-methoxycyclobutyl)-3-methyl-1,3-dihydro-2H-imidazo[4,5-c]cinnolin-2-one
8-(6-(1-(2-(3-azabicyclo[3.1.0]hexan-3-yl)ethoxy)-2,2,2-trifluoroethyl)pyridin-3-yl)-1-(3-methoxycyclobutyl)-3-methyl-1,3-dihydro-2H-imidazo[4,5-c]cinnolin-2-one
1-(3-methoxycyclobutyl)-3-methyl-8-(6-(2,2,2-trifluoro-1-(2-(piperidin-1-yl)ethoxy)ethyl)pyridin-3-yl)-1,3-dihydro-2H-imidazo[4,5-c]cinnolin-2-one
8-(6-(1-(2-(4,4-dimethylpiperidin-1-yl)ethoxy)-2,2,2-trifluoroethyl)pyridin-3-yl)-1-(3-methoxycycobutyl)-3-methyl-1,3-dihydro-2H-imidazo[4,5-c]cinnolin-2-one
1-(3-methoxycyclobutyl)-3-methyl-8-(6-(2,2,2-trifluoro-1-(2-(4-methoxypiperidin-1-yl)ethoxy)ethyl)pyridin-3-yl)-1,3-dihydro-2H-imidazo[4,5-c]cinnolin-2-one
8-(6-(1-(2-(6-azaspiro[2.5]octan-6-yl)ethoxy)-2,2,2-trifluoroethyl)pyridin-3-yl)-1-(3-methoxycyclobutyl)-3-methyl-1,3-dihydro-2H-imidazo[4,5-c]cinnolin-2-one
(R)-2-(1-(5-(1-isopropyl-3-methyl-2-oxo-2,3-dihydro-1H-imidazo[4,5-c]cinnolin-8-yl)pyridin-2-yl)ethoxy)acetic acid
8-(6-(2-(2-(3-azabicyclo[3.1.0]hexan-3-yl)ethoxy)propan-2-yl)pyridin-3-yl)-1-isopropyl-3-methyl-1H-imidazo[4,5-c]cinnolin-2(3H)-one
8-(6-((2-(3-azabicyclo[3.1.0]hexan-3-yl)-1,1-difluoroethoxy)methyl)pyridin-3-yl)-1-isopropyl-3-methyl-1H-imidazo[4,5-c]cinnolin-2(3H)-one
1-isopropyl-3-methyl-8-(6-(1-(2-(pyrrolidin-1-yl)ethoxy)propyl)pyridin-3-yl)-1H-imidazo[4,5-c]cinnolin-2(3H)-one
1-isopropyl-3-methyl-8-(6-(1-(2-(pyrrolidin-1-yl)ethoxy)butyl)pyridin-3-yl)-1H-imidazo[4,5-c]cinnolin-2(3H)-one
1-isopropyl-3-methyl-8-(6-(2-methyl-1-(2-(pyrrolidin-1-yl)ethoxy)propyl)pyridin-3-yl)-1H-imidazo[4,5-c]cinnolin-2(3H)-one, and
1-isopropyl-3-methyl-8-(6-(4-methyl-1-(2-(pyrrolidin-1-yl)ethoxy)pentyl)pyridin-3-yl)-1H-imidazo[4,5-c]cinnolin-2(3H)-one.

81. A pharmaceutical composition comprising a compound of Formula (I) or a pharmaceutically acceptable salt thereof, as claimed in claim 65, and at least one pharmaceutically acceptable excipient.

82. A method of treating ATM-associated diseases or conditions, preferably cancer, in a subject in need thereof, comprising administering to the subject a therapeutically effective amount of a compound of Formula (I) or a pharmaceutically acceptable salt thereof, as claimed in claim 65.

83. The method of claim 82, wherein the cancer is selected from the group consisting of colorectal cancer, glioblastoma, gastric cancer, ovarian cancer, diffuse large B-cell lymphoma, chronic lymphocytic leukaemia, acute myeloid leukaemia, head and neck squamous cell carcinoma, breast cancer, hepatocellular carcinoma, small cell lung cancer and non-small cell lung cancer.

84. The method of claim 82, wherein the compound of Formula (I) or a pharmaceutically acceptable salt thereof is administered simultaneously, separately or sequentially with radiotherapy, an anti-tumor agent such as antibody-toxin conjugate and topoisomerase inhibitor, or a PARP inhibitor selected from the group consisting of Olaparib, Rucaparib, Niraparib, Talazoparib, Pamiparib and Fluzoparib.
